(19) United States
(12) Patent Application Publication
(10) Pub. No.: US 2015/0309047 A1
(43) Pub. Date: Oct. 29, 2015

(54) CIRCULATING PROTEOLYTIC BIOMARKERS OF CELL DEATH AND METHODS FOR THE USE THEREOF

(71) Applicant: The Regents of the University of California, Oakland, CA (US)

(72) Inventors: James A. Wells, Burlingame, CA (US); Arun P. Wiita, San Francisco, CA (US)

(21) Appl. No.: 14/696,218

(22) Filed: Apr. 24, 2015

Related U.S. Application Data
(60) Provisional application No. 61/983,875, filed on Apr. 24, 2014.

(51) Int. Cl. G01N 33/68; C12N 15/115

(52) U.S. Cl. CPC G01N 33/6854; C12N 15/115; C12N 2310/16

(57) ABSTRACT

Provided herein are peptides useful, inter alia, for determining a level of apoptosis in a cancer patient. Further provided are complexes including said peptides bound to a binding reagent and antibodies specifically binding said peptides.

### CROSS-REFERENCE TO RELATED APPLICATIONS

This application claims the benefit of U.S. Provisional Application No. 61/983,875, filed Apr. 24, 2014, the content of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with Government support under Grant No. R01 CA154802 awarded by the National Institutes of Health. The Government has certain rights in this invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 48536-565001US_ST25.TXT, created on Apr. 23, 2015, 128,332 bytes, machine format IBM-PC, MS-Windows operating system, is hereby incorporated by reference.

### BACKGROUND OF THE INVENTION

For most patients with cancer, there is no current method to quickly distinguish if an administered chemotherapeutic is effective. As a result, many patients for whom a given drug is not effective are exposed to cytotoxic side effects without any benefit.

Cytotoxic chemotherapy is the mainstay of many cancer treatments. However, for the majority of cancers, there are no specific diagnostic tests to rapidly assess whether a given chemotherapy is effective. Instead, patients must wait weeks to months for expensive imaging studies to determine if chemotherapy was effective (Fletcher J W, et al. (2008) Recommendations on the use of 18F-FDG PET in oncology. J Nucl Med 49(3):480-508). Ineffective treatment subjects patients to unnecessary cytotoxic side effects, critical delays in identifying an efficacious therapeutic regimen, and unnecessary use of costly cancer drugs. Therefore, there is a distinct clinical need for rapid, inexpensive biomarkers of chemotherapeutic efficacy to optimize cancer management.

It is known that that the majority of small molecule chemotherapeutics function by inducing apoptosis in cancer cells (see, for example, Kaufmann S H & Earnshaw W C (2000) Induction of apoptosis by cancer chemotherapy. Exp Cell Res 256(1):42-49). Both in vitro and in vivo, apoptosis occurs rapidly, typically within 6-72 hours after exposure to a chemotherapeutic (see, for example, Blankenberg F G (2008) In vivo imaging of apoptosis. Cancer Biol Ther 7(10):1525-15323; or Renz A, et al. (2001) Rapid extracellular release of cytochrome c is specific for apoptosis and marks cell death in vivo. Blood 98(5):1542-1548). Many proteases are activated during apoptosis (see, for example, Moffitt K L, Martin S L, & Walker B (2010) Proteases implicated in apoptosis: old and new. J Pharm Pharmacol 62(5):563-576), though the key effectors are the caspases: cysteine proteases that cleave intracellular elements and lead to cell death (see, for example, Crawford E D & Wells J A (2011) Caspase substrates and cellular remodeling. Annu Rev Biochem 80:1055-1087). Using imaging approaches, caspase activity in tumors has been identified post-chemotherapy (see, for example, Yang T J, Haimovitz-Friedman A, & Verheij M (2012) Anticancer therapy and apoptosis imaging. Exp Oncol 34(3):269-276). Furthermore, through an unknown mechanism, some intracellular protein contents are known to be released from apoptotic tumor cells into the bloodstream; such intracellular protein contents include histones, cytochrome c, and a caspase-cleaved fragment of the intermediate filament protein cytokeratin 18 (see, for example, Beachy S H & Repasky E A (2008) Using extracellular biomarkers for monitoring efficacy of therapeutics in cancer patients: an update. Cancer Immunol Immunother 57(6):759-775; Greystoke A, et al. (2011) Assessment of circulating biomarkers for potential pharmacodynamic utility in patients with lymphoma. Br J Cancer 104(4):719-725; or Olofsson M H, et al. (2007) Cytokeratin-18 is a useful serum biomarker for early determination of response of breast carcinomas to chemotherapy. Clin Cancer Res 13(11):3198-3206). However, these few existing markers unfortunately do not show sufficient sensitivity and specificity for use as clinical diagnostics of chemotherapeutic efficacy (see, for example, Dean E, Greystoke A, Ranson M, & Dive C (2012) Biomarkers of cell death applicable to early clinical trials. Exp Cell Res 318(11):1252-1259). The discovery of broad signatures of apoptosis, beyond the handful of markers already known, may offer sufficient diagnostic power to clinically monitor therapeutic response and greatly benefit cancer management.

The 600 or so proteases encoded in the human genome are involved in a diversity of biological processes. Some function as nonspecific degradative enzymes associated with protein catabolism, indiscriminately and exhaustively cleaving many protein substrates at many sites. In contrast, several others function as selective post-translational modifiers, cleaving a limited set of protein substrates, usually at only one or a few sites. Apoptosis is an important example of a biological process regulated by widespread but specific intracellular proteolysis, predominantly carried out by the caspase family of proteases. This genetically programmed and non-inflammatory form of cell death is a central component of homeostasis, tissue turnover, and development. Since apoptotic turnover of cells lies in direct opposition to the uncontrolled growth of tumor cells, a strong link also exists between apoptosis and cancer. Indeed, the terminal cellular effect of most chemotherapeutic compounds is induction of apoptosis (Kaufmann et al., Exp Cell Res, 2000, 256, 42-9).

The widespread intracellular proteolysis that is a hallmark of apoptosis is predominantly mediated by a family of aspartate-specific proteases termed caspases (Taylor et al., Nat Rev Mol Cell Biol, 2008, 9, 231-41). Apoptosis can be induced by extracellular death ligands, such as Fas ligand, TNF-α, or TRAIL, via the extrinsic pathway to activate caspase-8. It can also be induced by agents such as cytotoxic compounds, radiation, and other environmental stresses via the intrinsic pathway with release of proapoptotic factors from mitochondria to activate caspase-9. Initiator caspases-8 and -9 in turn activate downstream executioner caspases, among them caspases-3 and -7. Caspases then catalyze the inactivation of a multitude of prosurvival/antiapoptotic proteins and activation of antisurvival/proapoptotic proteins. The combined proteolytic events culminate in apoptotic cell death and clearance by phagocytes.

As a specific illustration, after receiving a cell death signal, apoptotic cells execute a cellular program that results in widespread and dramatic cellular changes that can include:
(1) cell shrinkage and rounding due to the breakdown of the proteinaceous cytoskeleton;
(2) the appearance of a dense cytoplasm and tight packing of cell organelles;
(3) chromatin condensation into compact patches against the nuclear envelope;
(4) discontinuity of the nuclear envelope and DNA fragmentation;
(5) breakdown of the nucleus into several discrete chromatin bodies or nucleosomal units due to the degradation of DNA;
(6) blebbing of the cell membrane into irregular buds.
   Near the conclusion of the apoptotic program, the cell breaks apart into several vesicles called apoptotic bodies, which are then typically phagocytosed.

Because the study of apoptotic pathways has ramifications for development of therapies for treatment of cancer, there is significant interest in gaining a better understanding of caspase proteolysis during apoptosis. For example, identification of new targets of proteolysis in apoptosis can lead to discovery of prosurvival/antiapoptotic factors, which can in turn serve as novel targets for cancer chemotherapy. A number of caspase substrates are active or established drug targets for treating cancer, including topoisomerases I and II, androgen receptor, thymidylate synthase, Bcl-2, IAPs, Mdm2 or Hdm2, PARP, HSP90, HDACs, the proteasome, Aid, MEK, Abl, EGFR, HER2, and VEGF, to name a few.

Products of caspase proteolysis may also serve as useful biomarkers of in vivo apoptosis. For example, serum levels of the caspase cleavage product of cytokeratin-18 have been used as a marker of chemotherapeutic efficacy in prostate, breast, and testicular cancers (Kramer et al., Br J Cancer, 2006, 94, 1592-8; Olofsson et al, Clin Cancer Res, 2007, 13, 3198-208; de Haas et al, Neoplasia, 2008, 10, 1041-8). Although apoptotic cells are typically cleared by phagocytes such as macrophages, it has been hypothesized that local clearance mechanisms are overloaded in cases of high cellular turnover and death, causing dying apoptotic cells to undergo secondary necrosis (Linder et al, Cancer Lett, 2004, 1, 1-9). While the plasma membrane remains intact during apoptosis, it is compromised and ruptured during secondary necrosis. Such secondary necrosis of dying tumor cells is consistent with the observation of what are normally intracellular components such as cytochrome c, DNA, nucleosomes, and cytokeratin-18 in the vasculature of cancer patients during chemotherapy (Beachy et al., Cancer Immunol Immunother, 2008, 57, 759-75).

A logical extension of these findings is that other caspase-derived neo-epitopes, besides caspase-cleaved cytokeratin-18, are released into the vasculature of cancer patients undergoing chemotherapy. Such additional caspase-proteolyzed proteins may represent novel prognostic, diagnostic, or pharmacodynamic biomarkers of in vivo apoptosis, predicting likely patient outcome, indicating the most suitable therapeutic regimen, or serving as markers of therapeutic response. Because of tumor and patient heterogeneity, the clinical utility of single biomarker assays can be limited (Anderson et al., Mol Cell Proteomics, 2002, 1, 845-67). A multiparameter diagnostic assay of in vivo apoptosis based on a panel of caspase-derived neo-epitopes would likely be more sensitive and specific for a given type of cancer or therapeutic regimen. Great utility therefore exists in the identification of physiologically relevant caspase cleavage sites. Knowledge of such cleavage sites is required for the preparation of both peptide standards corresponding to neo-epitopes and antibodies that specifically bind to neo-epitopes. These reagents will enable identification and quantitation of caspase-derived neo-epitopes in biological samples such as serum, plasma, or tissue biopsies, and for validation of a given set of caspase-derived neo-epitopes as clinically useful biomarkers of in vivo apoptosis.

### BRIEF DESCRIPTION OF THE INVENTION

In accordance with the present invention, using a unique enzymatically-driven technology, it has been found that numerous protein fragments are released into the bloodstream post-chemotherapy from apoptotic cancer cells. These circulating signatures of cell death in hematologic malignancy patients may form the foundation for entirely novel, rapid, and inexpensive biomarkers of chemotherapeutic efficacy. Such biomarkers could transform management of many cancer types.

Thus, while it may be that many more intracellular contents are likely to be released into the bloodstream after chemotherapy-induced apoptosis, such intracellular contents cannot be readily identified with existing technologies. However, the use of the recently developed unique, single step labeling technology, using the enzyme subtiligase, allowed for the positive enrichment of free protein N-termini generated by proteolysis followed by mass spectrometry (MS)-based identification (see, for example, Mahrus S, et al. (2008) Global sequencing of proteolytic cleavage sites in apoptosis by specific labeling of protein N termini. Cell 134(5):866-876). This approach has particular advantages in blood plasma analysis as it largely avoids interference from high-abundance proteins such as albumin, allowing for the identification of low-abundance species which could not be found through traditional plasma proteomics (see, for example, Wildes D & Wells J A (2010) Sampling the N-terminal proteome of human blood. Proc Natl Acad Sci USA 107(10):4561-4566).

Thus, according to one aspect of the present invention, this N-terminomics method was used to identify proteolytic fragments in the peripheral blood of hematologic malignancy patients within 24 h of chemotherapy induction. In these plasma samples, a number of caspase-cleaved and other proteolytically generated fragments not previously found in normal plasma were first identified. Remarkably, many of these same proteolytic products were also released from hematologic malignancy cells in culture treated with clinically relevant chemotherapeutics. Ultimately, using a combination of unbiased and targeted liquid chromatography-tandem MS (LC-MS/MS) approaches, over 150 N-terminal fragments were identified in post-chemotherapy plasma derived from proteins not found in normal blood. These findings greatly expand the known library of proteolytic products released from dying cells.

Furthermore, a quantitative MS assay has been developed which allows confirmation that many of these N-termini are indeed increased in abundance post- vs. pre-chemotherapy in a larger cohort of hematologic malignancy patients. Overall, these results provide initial evidence that unbiased monitoring of proteolysis is a promising and novel strategy to rapidly assess chemotherapeutic efficacy in cancer patients.

Thus, the present invention relates generally to isolated polypeptides comprising a proteolytic peptide generated in response to an apoptotic stimulus in mammalian cells, provided, however, that said polypeptides do not include any of the sequences set forth in Table 1. In certain embodiments of the invention, there are provided 153 experimentally determined and physiologically relevant caspase-like cleavage sites, a method for discovering additional physiologically relevant caspase cleavage sites, discovery of biomarkers of in vivo apoptosis based on any of these caspase-like cleavage sites, and methods and compositions for detecting and quantitating protein neo-epitopes corresponding to these biomarkers in biological samples, using either peptide standards and mass spectrometry, or antibodies specific to neo-epitopes, or both. Further provided herein are in vitro polypeptide complexes including the biomarkers provided herein bound to a binding reagent (e.g., an antibody of aptamer). Further provided are antibodies capable of binding the biomarkers provided herein including embodiments thereof. The invention also provides compositions and kits for performing the methods of the invention.

Direct and selective labeling of protein α-amines or α-carboxylates is a powerful approach for profiling proteolysis in complex mixtures since it permits direct identification of cleavage sites in protein substrates. Approximately 80% of mammalian proteins are known to be N-terminally acetylated (Brown et al., J Biol Chem. 1976; 251(4):1009-14). Thus, greater signal over background can be achieved through N-terminal instead of C-terminal labeling. However, such labeling must still be extremely selective for α-amines over lysine g-amines, which are approximately 25 times more abundant in an average protein. To achieve this selectivity, an enzymological approach that makes use of the rationally designed protein ligase subtiligase has been adopted. This engineered enzyme exhibits absolute selectivity for modification of α-amines (see, for example, Abrahmsén et al., Biochemistry. 1991; 30(17):4151-9; and Chang et al., Proc Natl Acad Sci USA. 1994; 91(26):12544-8).

In accordance with one aspect of the present invention, a proteomic method has been developed utilizing subtiligase that enables capture and sequencing of N-terminal peptides found in complex biochemical mixtures (see US Publication No. 2012-0028266 A1). Proteins in biological samples are N-terminally biotinylated by treatment with subtiligase and peptide glycolate ester substrates specially tailored to the proteomic workflow. Biotinylated samples are exhaustively digested with trypsin, and N-terminal peptides are captured using avidin affinity media. The peptide ester substrate contains a tobacco etch virus (TEV) protease cleavage site to permit facile recovery of captured peptides. An important aspect of the workflow is that recovered peptides retain an N-terminal serinyl-tyrosyl dipeptide modification or 2-aminobutyryl modification, providing a key hallmark to distinguish labeled peptides from contaminating unlabeled peptides using tandem mass spectrometry (LC/MS/MS). In standard protease nomenclature, substrates are cleaved between the P1 (N-terminal) and P1′ (C-terminal) residues, with Pn and Pn′ residues increasing in count by one in both directions away from the scissile bond (Schechter and Berger, 1968). Thus, the Pn′ residues of a cleavage site correspond to N-terminal residues of the labeled peptide identified, while the Pn residues of a cleavage site can be inferred from the protein sequence preceding the identified peptide.

Over 300 publications describing a wide variety of cell types and apoptotic inducers have reported the proteolysis of approximately 360 human proteins in apoptosis, but only approximately 300 caspase cleavage sites in human protein substrates have been reported (Liithi et al., Cell Death Differ. 2007; 14(4):641-50). In accordance with one aspect of the present invention, studies have been carried out in a number of cancer cell lines, including Jurkat, an acute lymphocytic leukemia cell line, DB, a diffuse large B cell lymphoma cell line, and RPMI 8228, a multiple myeloma cell line, using a variety of apoptotic inducers including etoposide, doxorubicin, staurosporine, and TRAIL. These combined studies to date have resulted in the identification of a large number of caspase cleavage sites in a number of different protein substrates. These caspase cleavage sites and additional caspase cleavage sites yet to be discovered in other model systems of human cancers represent a wealth of knowledge and an excellent starting point for discovery of novel biomarkers of in vivo apoptosis, and for preparation of reagents for detection and quantitation of such biomarkers in biological samples.

In certain aspects, the present invention provides proteolytic polypeptide biomarkers for the detection and quantitation of apoptosis, provided, however, that said polypeptide does not include any of the sequences set forth in Table 1. In one embodiment of the invention, these biomarkers comprise proteolytic polypeptides generated in response to an apoptotic stimulus. The biomarkers of the present invention may be generated in response to a specific apoptotic stimulus or conversely may be generated by multiple or general apoptotic stimuli. In some embodiments, the proteolytic polypeptide biomarkers of the present invention are generated by the action of a single protease, or by the action of a limited set of proteases activated in response to a specific apoptotic stimulus. In other embodiments, the biomarkers may be generated by the action of a plurality of apoptotic proteases. In a particular embodiment, the proteolytic apoptotic polypeptide biomarkers comprise N-termini or C-termini selected from those found in Table 4.

In one embodiment, the proteolytic biomarkers of the present invention are useful for the detection of apoptosis in an individual. In a specific embodiment, the proteolytic biomarkers are useful for the diagnosis in an individual of a disease characterized by apoptosis. In another embodiment, these biomarkers are useful for providing a prognosis for an individual suffering from a disease characterized by apoptosis. In yet other embodiments, these biomarkers are useful for determining the extent of apoptosis in an individual or the severity, stage, or other relevant characteristics of a disease characterized by apoptosis in an individual. In one particular embodiment, the proteolytic apoptotic biomarkers of the present invention are useful in determining the efficacy of a drug in vitro or in vivo.

In another embodiment, the present invention provides novel proteolytic apoptotic cleavage junctions. In certain embodiments, these cleavage junctions comprise amino acids that are cleavage substrates for proteases activated in response to an apoptotic stimulus. In a particular embodiment, the proteolytic apoptotic cleavage junctions comprise an amino acid sequence selected from those found in Table 4. In a first embodiment, the cleavage junctions of the present invention are useful for detecting apoptosis in a biological sample. In a second embodiment, the cleavage junctions are useful for diagnosing or providing a prognosis for a disease state associated with apoptosis in an individual, or for assessing response to a particular line of therapy. For instance, a protein or polypeptide comprising the cleavage junction can be used in an assay to measure apoptotic protease (e.g., a caspase) activity or levels in a sample. The peptides or polypeptides comprising the junction may be of a variety of lengths, preferably from 7 to 40, 7 to 20, or 10 to 30 amino acids in length.

The present invention also provides proteolytic apoptotic signatures. In one embodiment, the apoptotic signatures of the invention comprise at least one proteolytic polypeptide generated in response to an apoptotic stimulus. In another embodiment of the invention, the apoptotic signatures comprise the levels of one or more proteolytic polypeptides. In a particular embodiment, the apoptotic signatures of the present invention comprise the presence or particular level of one or more proteolytic polypeptides comprising N-termini or C-termini selected from those found in Table 4.

In yet other embodiments, the apoptotic signatures of the present invention may comprise one or more ratios of cleaved to uncleaved apoptotic proteolytic sites. In a particular embodiment of the present invention, the apoptotic proteolytic sites are selected from those found in Table 4. In some embodiments, the proteolytic apoptotic signatures of the present invention may correspond to the presence or absence of a disease state in an individual. In other embodiments of the present invention, the proteolytic apoptotic signatures may correspond to a particular level of apoptosis in an individual or in a sample from an individual suffering from a disease characterized by apoptosis. In another embodiment of the present invention, the proteolytic apoptotic signatures may correspond to a prognosis for an individual suffering from a disease characterized by apoptosis. In yet other embodiments, the apoptotic signatures may correspond to a level of efficacy for a drug or to a response level in an individual taking a drug or receiving a treatment for a disease characterized by apoptosis.

In one embodiment, the present invention provides reagents for detecting the proteolytic apoptotic polypeptide biomarkers of the invention. In one embodiment, the reagents comprise synthetic peptides corresponding to an N-terminal or C-terminal sequence selected from those found in Table 4. In a particular embodiment, these synthetic peptides have the same sequence as either an unmodified or modified peptide found in Table 4. In another embodiment, these synthetic peptides contain six or more consecutive residues from a sequence of previous residues found in Table 4, starting from the most C-terminal residue, and possibly extending to further than eight prior residues in the sequence of the full-length protein. In one embodiment, these peptides correspond to the most N-terminal peptide obtained after digestion with trypsin of the C-terminal fragment of the full-length protein following proteolysis during apoptosis at one of the cleavage sites found in Table 4.

In another embodiment, these peptides correspond to the most C-terminal peptide obtained after digestion with trypsin of the N-terminal fragment of the full-length protein following proteolysis during apoptosis at one of the cleavage sites found in Table 4. In another embodiment, these peptides correspond to the peptides that would be obtained following digestion of the N- and C-terminal fragments of the protein substrate with a protease other than trypsin, including, but not limited to, chymotrypsin, V8, Lys-C, Lys-N, Arg-C, Asp-N, Asp-C, pepsin, and thermolysin. In another particular embodiment, these peptides correspond to the peptides that would be obtained following treatment of the N- and C-terminal fragments of the protein substrate with a chemical cleavage agent such as cyanogen bromide. In a specific embodiment, the synthetic peptides contain stable heavy isotopes of carbon or nitrogen (e.g., ¹³C or ¹⁵N), incorporated by use of the appropriately heavy isotope-labeled amino acid during preparation of the synthetic or modified peptides. In a particular embodiment, the light and heavy versions of the peptides are used as standards in a mass spectrometry approach such as selected reaction monitoring (SRM) or multiple reaction monitoring (MRM) to optimize detection of corresponding peptides in biological samples derived from proteolytic apoptotic polypeptides, and to permit quantitation of such peptides in biological samples.

In another embodiment, the present invention provides reagents for detecting the proteolytic apoptotic polypeptide biomarkers of the invention. In one embodiment, the reagents comprise proteins that bind to the biomarkers with high affinity and specificity. In a particular embodiment, the reagents comprise antibodies, or fragments thereof, generated against the proteolytic apoptotic polypeptides of the present invention. In a specific embodiment, the present invention provides antibodies that bind to a proteolytic apoptotic polypeptide comprising an N-terminal or C-terminal sequence selected from those found in Table 4. In one embodiment, an antibody of the present invention binds to the target proteolytic fragment, but does not substantially bind to the full-length protein or intact proteolytic cleavage junction. In other embodiments, the reagents comprise antibodies generated against antigens comprising apoptotic cleavage sites or junctions. In a specific embodiment, the present invention provides antibodies that bind to an apoptotic cleavage site selected from those listed in Table 4. In one embodiment, the antibodies of the present invention bind to an intact proteolytic cleavage junction, but do not substantially bind to the N-terminal or C-terminal proteolytic polypeptide generated in response to an apoptotic stimulus. In another particular embodiment of the invention, antibodies are provided that bind to the N-terminus or C-terminus of a proteolytic polypeptide comprising a sequence selected from those found in Table 4.

In another embodiment, the present invention provides methods of generating binding reagents to one or more proteolytic apoptotic polypeptide biomarker. In one embodiment of the invention, methods are provided for generating a binding reagent to a single proteolytic polypeptide. In other embodiments, the present invention provides methods of simultaneously generating binding reagents against more than one proteolytic polypeptide of the present invention. In a particular embodiment, the present invention provides methods of generating antibodies against one or more proteolytic apoptotic polypeptide of the invention.

In one embodiment, the present invention provides methods of detecting apoptosis or determining the level of apoptosis in an individual or in a sample from an individual. In one embodiment, the methods comprise detecting a proteolytic apoptotic polypeptide biomarker generated in response to an apoptotic stimulus in a biological sample. In certain embodiments, the methods of the present invention comprise detecting one or more biomarkers comprising an N-terminal or C-terminal sequence selected from those found in Table 4. In other embodiments of the present invention, methods are provided for detecting or determining a proteolytic apoptotic signature. In certain embodiments of the invention, detecting or determining a proteolytic apoptotic signature comprises detecting or determining the level of one or more proteolytic apoptotic polypeptide biomarkers generated in response to an apoptotic signature. In other embodiments, the methods further comprise comparing a first proteolytic apoptotic signature detected in an individual with a second apoptotic signature corresponding to a predetermined apoptotic level or disease state. In certain embodiments of the invention, said second apoptotic signature comprises an average or conglomerate apoptotic signature determined from samples taken from a plurality of individuals suffering from the same disease or disease state associated with apoptosis. In yet other embodiments, the methods of the present invention comprise determining the ratio of the levels of at least one proteolytic apoptotic polypeptide to the levels of at least one intact proteolytic cleavage junction.

In another embodiment, the present invention provides methods for diagnosing or providing a prognosis for a disease associated with apoptosis in an individual, or for tracking therapeutic progress in an individual. In some embodiments of the present invention, the methods comprise detecting one or more proteolytic apoptotic polypeptide biomarkers in a sample from said individual. In other embodiments, the methods of the present invention comprise detecting a proteolytic apoptotic signature in a sample from an individual. In particular embodiments, the methods of the present invention comprise comparing the level of one or more proteolytic apoptotic polypeptide or apoptotic signature in an individual with one or more proteolytic apoptotic signature corresponding to a predetermined disease or disease state. In yet other embodiments, the methods of diagnosing and providing a prognosis provided by the present invention comprise determining the ratio of the levels of at least one proteolytic apoptotic polypeptide to the levels of at least one intact proteolytic cleavage junction. In particular embodiments, these methods further comprise comparing said ratios to predetermined values corresponding to a particular diagnosis or prognosis for a disease state associated with apoptosis. In another embodiment, the methods comprise comparing levels of apoptotic signatures in a patient before the start of therapy, and during the course of therapy.

In one embodiment, the present invention provides kits for use in the detection of proteolytic apoptotic polypeptides. In some embodiments, the kits of the present invention comprise a plurality of light- or heavy-labeled synthetic peptides corresponding to N- and/or C-terminal sequences found in Table 4 that can be used for optimizing detection of corresponding peptides in biological samples derived from proteolytic apoptotic polypeptides, and to permit quantitation of such peptides in biological samples, using mass spectrometry. In other embodiments, the kits of the present invention comprise a plurality of binding reagents that specifically bind to proteolytic polypeptides that are generated in response to an apoptotic stimulus. In a specific embodiment, the kits of the present invention comprise a plurality of binding reagents that bind to polypeptides comprising an N-terminal or C-terminal sequence found in Table 4. In certain embodiments, the binding reagents are antibodies, including polyclonal antibodies, monoclonal antibodies, and fragments thereof.

In certain embodiments, the kits of the present invention are useful in the diagnosis or prognosis of a disease characterized by apoptosis in an individual, or for tracking therapeutic progress in an individual that is characterized by an increased level of apoptosis. In yet other embodiments, the present invention provides kits comprising a plurality of binding reagents that specifically bind to proteolytic apoptotic cleavage junctions. In a particular embodiment, the proteolytic apoptotic cleavage junctions comprise amino acid sequences found in Table 4. In still other embodiments, the kits of the present invention comprise at least one binding reagent that specifically binds to a proteolytic apoptotic polypeptide and at least one binding reagent that specifically binds to a proteolytic apoptotic cleavage junction. In other certain embodiments, the kits comprise binding reagents that specifically bind to peptides generated from proteolytic apoptotic polypeptides after treatment with proteases such as trypsin, chymotrypsin, V8, Lys-C, Lys-N, Arg-C, Asp-N, Asp-C, pepsin, or thermolysin, or reagents such as cyanogen bromide, permitting enrichment of these peptides for detection and quantitation using mass spectrometry.

In another aspect, the invention provides a method of modulating apoptosis by administering siRNA or shRNA corresponding to an mRNA encoding a protein of Table 4. In this first aspect, the invention also provides a pharmaceutical composition comprising the siRNA molecule or the shRNA molecule and/or an siRNA or shRNA expression vector which comprises a portion of a nucleotide sequence complementary to an mRNA encoding a protein of Table 4. In some embodiments, the siRNA is at least about 15-50 nucleotides in length (e.g., each complementary sequence of the double stranded siRNA is 15-50 nucleotides in length, and the double stranded siRNA is about 15-50 base pairs in length). In some further embodiments still, the length of the siRNA molecule is about about 20-30 base nucleotides, about 20-25 or about 24-29 nucleotides in length, e.g., 20, 21, 22, 23, 24, 25, 26, 27, 28, 29, or 30 nucleotides in length. In still further embodiments, the siRNA is a small hairpin loop or small hairpin RNA, known as shRNA. In some embodiments, the invention provides a method of treating cancer or inducing apoptosis in a subject in need thereof by administering the siRNA or shRNA or siRNA vector or shRNA vector to the subject. In some embodiments of any of the above the siRNA or shRNA is directed toward a protein having an M value from Table 4 greater than 1, 2, 4, or 8. In other embodiments, siRNA corresponding to a protein of Table 4 having a plurality of such cleavage sites is used.

In one aspect, a method of determining a level of a peptide in a cancer subject is provided. The method includes (i) assaying a biological sample from a cancer subject; and (ii) determining a level of a peptide of (consisting of) SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in the biological sample.

In another aspect, a method of determining apoptosis in a subject is provided. The method includes (i) detecting a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in a biological sample from a subject receiving or having received a therapeutic agent, wherein the detecting includes contacting a binding reagent with the peptide to form a binding reagent peptide complex and detecting the binding reagent peptide complex. The level is compared to a standard control, thereby determining apoptosis in a subject.

In another aspect, a method of determining efficacy of a therapeutic agent in a subject is provided. The method includes (i) detecting a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in a sample from a subject receiving a therapeutic agent, wherein the detecting includes contacting a binding reagent with the peptide to form a binding reagent peptide complex and detecting the binding reagent peptide complex. (ii) It is determined whether the level is increased relative to a standard control, wherein an elevated level of a peptide relative to the standard control indicates efficacy of the therapeutic agent. And (iii) based at least in part on the level in step (ii), determining efficacy of the therapeutic agent.

In another aspect, an in vitro polypeptide complex is provided. The in vitro polypeptide complex includes a peptide bound to a binding agent, wherein the peptide is SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552.

In another aspect, a conjugate is provided. The conjugate includes a peptide covalently bound to a detectable moiety, wherein the peptide is SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552.

In another aspect, an antibody or aptamer that specifically binds to SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 is provided.

### BRIEF DESCRIPTION OF THE DRAWINGS

FIG. 1A, sheet 1, presents a subtiligase subtiligase-based method for positive selection of peptides corresponding to N-termini of proteins from complex mixtures. FIG. 1A, sheet 1 specifically sets forth the workflow for biotinylation of protein N-termini in complex mixtures using subtiligase and a biotinylated peptide ester that contains a TEV protease cleavage site, trypsinization of labeled proteins, capture of biotinylated N-terminal peptides with immobilized avidin, recovery of captured peptides using TEV protease, and analysis of N-terminal peptides by 1D or 2D LC/MS/MS for identification of corresponding proteins and cleavage sites. The representative MS/MS spectrum presented in FIG. 1A, sheet 2, corresponds to semi-tryptic peptide GSAVNGTSSAETNLEALQK from MEK1 (MP2K1_HUMAN) (SEQ ID NO:178) and identifies a previously unknown caspase-like cleavage site at Asp 16. The a² and b₂ ions at m/z 223 and 251 are characteristic hallmarks of a ligated, serinyl-tyrosyl dipeptive-bearing, N-terminal peptide.

FIG. 1B (sheets 1 and 2) present the structure of two biotinylated peptide glycolate esters used in the proteomic workflow. Sequences: SYGSAVNGTSSAETNLEALQK (SEQ ID NO:431); MEK1 protein sequence (SEQ ID NO:432); cleavage site (SEQ ID NO:433), Ester1 (SEQ ID NO:434), Ester2 (SEQ ID NO:435), TENLYFQSY (fragment of SEQ ID NO:434).

FIG. 2: Panel A, B and C present the classification of unique N-termini identified in untreated and apoptotic Jurkat cells according to Swiss-Prot annotation. FIG. 2 panel A presents the classification of N-termini identified in small-scale and large-scale experiments with untreated cells (131 and 661 unique N-termini, respectively, combined from two experiments in both cases). FIG. 2 panel B presents the classification of N-termini identified in small-scale experiments with untreated cells (131 unique N-termini combined from two experiments) and apoptotic cells (244 unique N-termini combined from four experiments). FIG. 1 panel C presents the classification of N-termini identified in large-scale experiments with untreated cells (661 unique N-termini combined from two experiments) and apoptotic cells (733 unique N-termini combined from three experiments).

FIG. 3 panel A and 3B illustrate that N-termini derived from caspase-like proteolytic processing are a hallmark of apoptotic cells. FIG. 3 panel A presents the frequencies of P1 and P1′ amino acid residues corresponding to non-homologous N-termini identified in small-scale 1D LC/MS/MS experiments with untreated and apoptotic Jurkat cells. Data are represented as mean±SD (n=2 for untreated and n=4 for apoptotic). FIG. 3 panel B illustrates the frequencies of P1 and P1′ amino acid residues corresponding to non-homologous N-termini identified in large-scale 2D LC/MS/MS experiments with untreated and apoptotic Jurkat cells. Data are represented as mean±SD (n=2 for untreated and n=3 for apoptotic). “-” indicates lack of a putative P1 residue in cases where the P1′ residue is an initiator methionine.

FIG. 4 identifies inferred P1 residues for all N-termini annotated in the human Swiss-Prot database originating from chain, signal peptide, transit peptide, or propeptide processing.

FIG. 5 panel A (sheet 1) and 5 panel B (sheet 2) present an analysis of proteolysis of selected proteins, all identified as caspase substrates in proteomic studies, during apoptosis in Jurkat cells following treatment with 50 μM etoposide. Black arrows indicate full-length proteins. Red arrows indicate expected cleavage products for cleavage at the sites identified in our studies. Cleavage products were not detected in all cases. FIG. 5 panel A presents time courses for the proteolysis of CCTδ, HDAC6, HDAC7, Ku80, LCOR, N-CoR, RBBP7, RCOR2, SHARP, TBLR1, UBPS, and UBP36 indicates full cleavage of HDAC6, HDAC7, N-CoR, RCOR2, SHARP, TBLR1, UBPS, and UBP36, and partial cleavage of CM, Ku80, LCOR, and RBBP7. FIG. 5 panel B illustrates the cleavage of a representative set of substrates identified in the present studies, HDAC7, Ku80, RCOR2, TBLR1, and UBP36, is blocked by the broad-spectrum caspase inhibitor Z-VAD(OMe)-fmk and is thus dependent on caspase activity.

FIG. 6 panel A-E illustrate the substrate specificity of the caspase-like proteolytic activity in etoposide-treated Jurkat cells. FIG. 6 panel A is a sequence logo representation (Crooks et al., 2004) of the frequency of amino acid residues in the identified caspase cleavage sites. FIG. 6 panel B is a sequence logo representation of the in vitro substrate specificity of caspase-3 (Stennicke et al., 2000; Thornberry et al., 1997). FIG. 6 panel C is a sequence logo representation of the frequency of amino acid residues in known human and human ortholog of rodent caspase cleavage sites (Liithi and Martin, 2007). FIG. 6 panel D indicates the frequency of P4-P1 motifs in the identified caspase cleavage sites. FIG. 6 panel E presents receiver operator characteristic curves showing the discrimination ability of HMMs constructed from three different cleavage site training sets (Jurkat, literature, and merged). Three representative HMM score threshold values for the merged dataset are indicates (TPR=true positive rate, FPR=false positive rate). Sequence: DEVD (SEQ ID NO:430).

FIG. 7 panel A-C are sequence logo representations of prototypical inflammatory, executioner, and initiator caspase substrate specificities. These are exemplified by FIG. 7 panel A caspase-1, FIG. 7 panel B caspase-3, and FIG. 7 panel C caspase-8, based on P4-P1 data adapted from Thornberry et al. (Thornberry et al., J Biol Chem. 1997; 272(29):17907-11) and P1′ data adapted from Stennicke et al. (Stennicke et al., Biochem J. 2000; 350 Pt 2:563-8).

FIG. 8 presents CID spectrum of the SY-labeled N-terminal peptide AAASAPQM(Oxidation)DVSK from N-CoR (NCOR1_HUMAN) (SEQ ID NO:402) corresponding to the P4-P4′ cleavage site LVD(1826)/AAAS (SEQ ID NO:403).

FIG. 9 presents CID spectrum of the SY-labeled N-terminal peptide GLSEQENNEK from N-CoR (NCOR1_HUMAN) (SEQ ID NO:404) corresponding to the P4-P4′ cleavage site EIID(385)/GLSE (SEQ ID NO:405).

FIG. 10 presents CID spectrum of the SY-labeled N-terminal peptide GTAEETEEREQATPR from N-CoR (NCOR1_HUMAN) (SEQ ID NO:406) corresponding to the P4-P4′ cleavage site DKID(555)/GTAE (SEQ ID NO:407).

FIG. 11 presents CID spectrum of the SY-labeled N-terminal peptide GDVEIPPNKAVVLR from TBLR1 (TBUR_HUMAN) (SEQ ID NO:408) corresponding to the P4-P4′ cleavage site MEVD(152)/GDVE (SEQ ID NO:409).

FIG. 12 presents CID spectrum of the SY-labeled homologous N-terminal peptide AVM(Oxidized)PDVVQTR from either TBLR1 (TBL1R_HUMAN) or TBL1X (TBL1X_HUMAN) (SEQ ID NO:410) corresponding to the P4-P4′ cleavage site SLID(86)/AVMP (SEQ ID NO:411).

FIG. 13 presents CID spectrum of the SY-labeled N-terminal peptide GGGPGQVVDDGLEHR from HDAC7 (HDAC7_HUMAN) (SEQ ID NO:412) corresponding to the P4-P4′ cleavage site LETD(412)/GGGP (SEQ ID NO:413).

FIG. 14 presents CID spectrum of the SY-labeled N-terminal peptide SIQEPVVLFHSR from SHARP (MINT_HUMAN) (SEQ ID NO:414) corresponding to P4-P4′ caspase-like cleavage site STTD(1574)/SIQE (SEQ ID NO:415).

FIG. 15 presents CID spectrum of the SY-labeled N-terminal peptide SDKGEFGGFGSVTGK from RBBP7 (RBBP7_HUMAN) (SEQ ID NO:416) corresponding to P4-P4′ caspase-like cleavage site SHCD(98)/SDKG (SEQ ID NO:417).

FIG. 16 panel A and panel B illustrate the invention approach to discovery of proteolytic biomarkers of cell death. Specifically, FIG. 16 panel A presents a general strategy for apoptotic biomarker discovery. Tumor cells rapidly undergo apoptosis in response to chemotherapeutic treatment. Proteolysis is activated during apoptosis and proteolytic fragments are released into the blood. Enzymatic labeling of free protein N-termini combined with identification and quantification mass spectrometry approaches identifies potential biomarkers of cell death.

FIG. 16 panel B illustrates the development of a pipeline for biomarker investigation. An initial discovery set of biomarkers is derived from MS experimentation on a set of “high-yield” patient plasma samples with significant decreases in circulating malignant cells after chemotherapy, studies in cell culture examining free N-termini released from cells into the media after chemotherapy, and an extensive database of intracellular proteolytic events during apoptosis. This discovery dataset is used to generate a targeted MS method to more sensitively detect intracellular content release into the plasma in high-yield patients. Finally, an additional patient cohort was collected for quantitative SRM MS to determine relative changes in proteolytic biomarkers before and after chemotherapy. Peptides reproducibly increased post-therapy serve as the most promising biomarkers of cell death for further clinical validation.

FIG. 17 panel A-C illustrate the identification of proteolytic fragments released post-chemotherapy in discovery and targeted MS. FIG. 17 panel A illustrates that high-yield hematologic malignancy patient cohort for initial discovery experiments all show large decreases in circulating malignant cells post-chemotherapy, suggesting extensive apoptosis directly in the peripheral blood.

FIG. 17 panel B indicates that from initial discovery MS experiments 98 unique N-terminal peptides were found in high-yield patient post-chemotherapy plasma derived from proteins not found in normal plasma (as listed in Wildes and Wells (Proc Natl Acad Sci USA 107(10):4561-4566). A number of overlapping peptides were found between proteolytic fragments released from apoptotic blood cancer cells in culture, further suggesting the fragments in blood are generated during cell death. FIG. 17 panel C presents eExample mass spectra for two biologically relevant markers of apoptosis using targeted MS on the LTQ Orbitrap Velos for patient NHL_{—}1.

FIG. 18 panel A and panel B illustrate the quantitative increases in proteolytic fragments after chemotherapy using a targeted SRM assay. FIG. 18 panel A presents example raw data from 140 peptide SRM assay for patient AML_{—}1. For each target peptide, four parent ion/fragment ion pairs (or “transitions”) are monitored for co-elution to confirm identification. Each trace represents intensity of a single transition. Total peptide intensity is the sum of area under the curve for all transitions. Three peptides (top row), from typical intracellular proteins, are greatly increased in the plasma post-chemotherapy. Of three peptides from typical plasma proteins (bottom row), only the caspase-cleaved fragment of gelsolin is greatly increased post-chemotherapy. FIG. 18 panel B illustrates that there are Logₑ fold-changes in peptide abundance post- vs. pre-chemotherapy in patient AML_{—}1. In contrast to plasma proteins, peptides derived from typical intracellular proteins show large changes in the blood post-chemotherapy, some increased over 50-fold.

FIG. 19 panel A and panel B demonstrate that quantitive increases in proteolytic peptides occur across a larger patient cohort. FIG. 19 panel A presents a heat map displaying subset of peptides with post- vs. pre-chemotherapy increases across 17 patients (log₂ fold-change in SRM peak area intensity; samples measured in duplicate, heat map value represents mean fold-change). Intriguingly, there is marked patient-to-patient variability, with some showing many increased peptides post-chemotherapy and others showing little change. D at P1 cleavage position (“(D)” before peptide name) indicates caspase proteolytic event. Grey indicates peptides not detected in SRM assay. FIG. 19 panel B presents example SRM data for peptides showing ˜2-fold intensity increases in additional patients.

FIG. 20 panel A-C provide confirmation and further analysis of monitoring proteolysis post-chemotherapy. FIG. 20 panel A presents an ELISA assay for full-length Smac protein and confirms ˜8.5-fold increase post-chemotherapy, as found by SRM assay, for patient AML_{—}1. The additional 12 patients tested did not have blood concentrations either pre- or post-treatment above the limit of quantification in this assay (1 ng/mL) so changes could not be determined.

FIG. 20 panel B illustrates that, although there is a statistically significant positive correlation (Pearson R=0.86, p<0.0001), there is a large variability between the decrease in circulating malignant cells post-chemotherapy at time of collection and the number of proteolytic peptides increased by SRM. Note the use of split axes to display results of patient AML_{—}1. FIG. 20 panel C indicates that the majority of proteolytic fragments released into the blood, based on the single cleavage site identified extended to the protein C-terminus, have molecular weights below that of serum albumin. Therefore they may be rapidly filtered into the urine, leaving a short time window for detection between induction of apoptosis and clearance. Proteolytic fragments are ranked by predicted molecular weight.

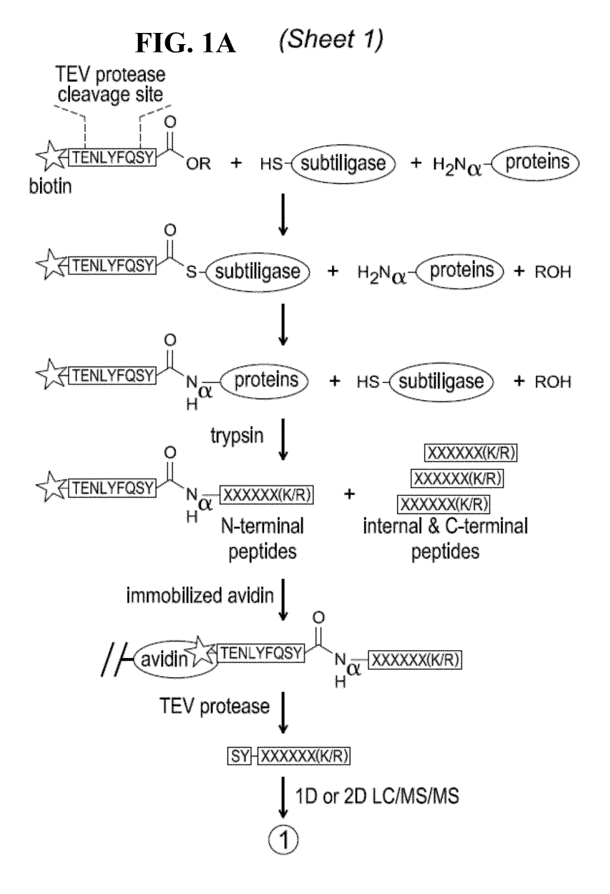

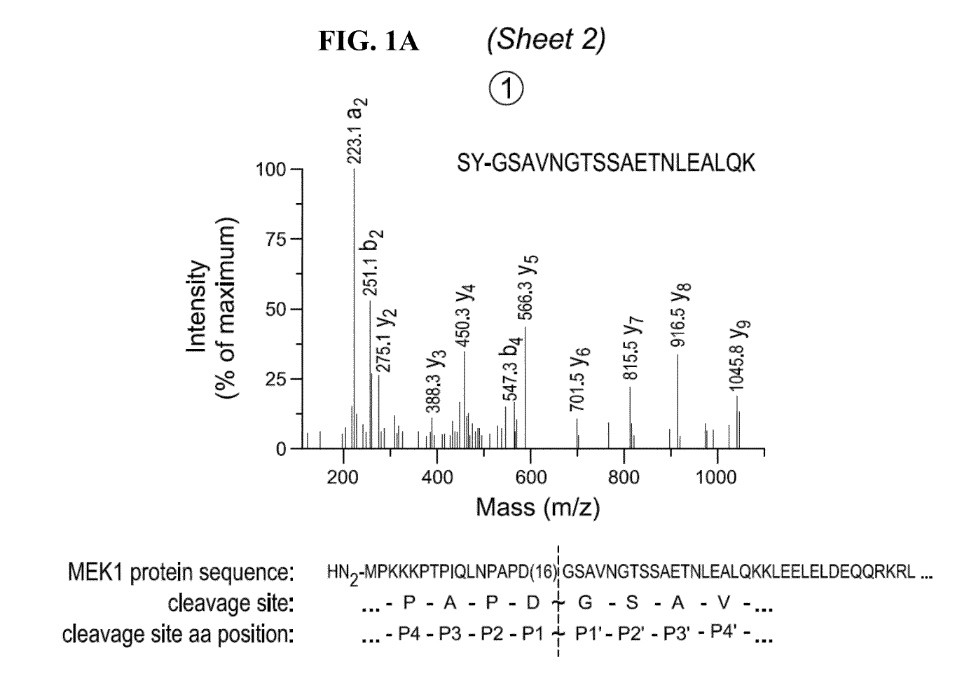

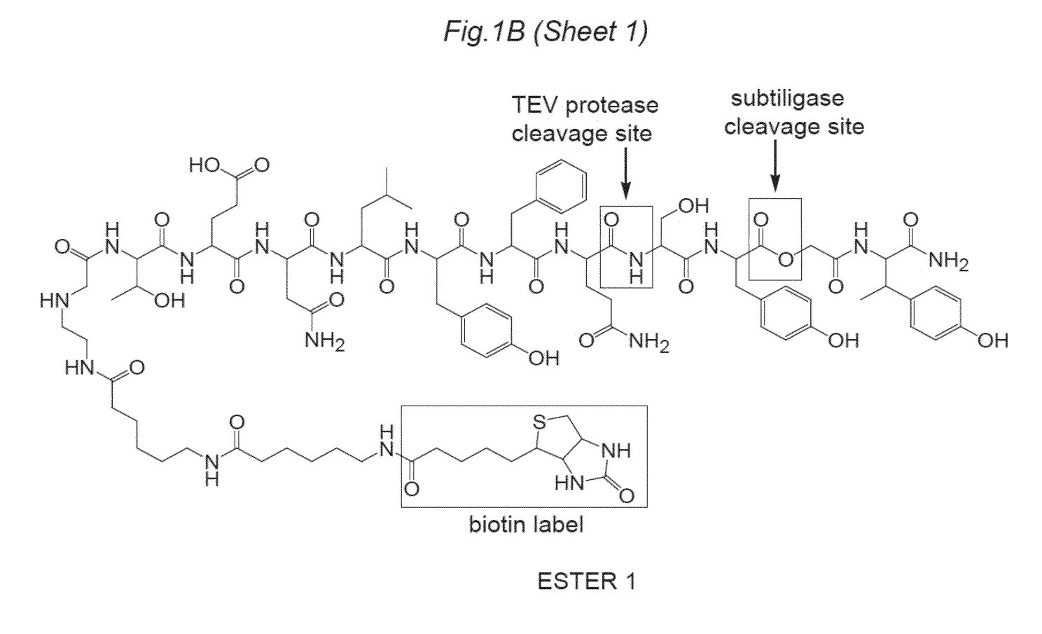

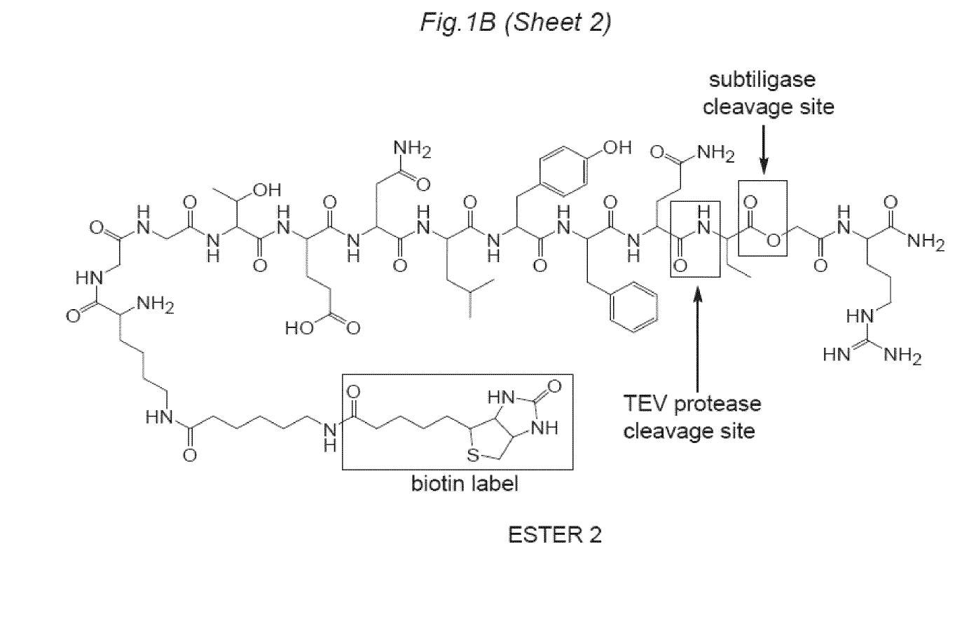

### DETAILED DESCRIPTION OF THE INVENTION

The present invention provides novel proteolytic apoptotic polypeptide biomarkers, provided, however, that said polypeptide does not include any of the sequences set forth in Table 1. In one embodiment of the invention, the proteolytic apoptotic polypeptide biomarkers are generated in response to an apoptotic stimulus. In certain embodiments, the apoptotic stimulus may be endogenous to the cell, tissue, organ, or organism of interest. In other embodiments, the apoptotic stimulus may be exogenous or induced, such as in tissue culture. In some embodiments, apoptosis may be induced by the treatment of cells, tissues, organs, or organisms with a drug known to cause apoptosis, such as etoposide, camptothecin, anisomycin, and the like. In a specific embodiment, the proteolytic apoptotic polypeptide biomarkers of the present invention comprise N-terminal or C-terminal sequences selected from those found in Table 4.

In certain embodiments of the invention, the proteolytic apoptotic polypeptide biomarkers comprise proteolytic fragments that are generated by cleavage of a full length protein of Table 4 or an intact proteolytic apoptotic cleavage junction of Table 4 by the action of a suitable protease. Suitable proteases will be readily apparent to the skilled artisan. In one particular embodiment, the protease is an enzyme known to function in the apoptotic pathway of a cell such as a caspase. In one embodiment of the present invention, a proteolytic apoptotic polypeptide biomarker of the present invention will have a sequence selected from those found in Table 4 at its N-terminus or C-terminus. In some embodiments of any of the above the polypeptide biomarker corresponds to a protein having an M value from Table 4 of 1 or greater than 1, 2, 4, or 8. In other embodiments, the biomarker corresponds to a protein of Table 4 having a plurality of such apoptotic polypeptide biomarkers or cleavage sites. In yet another embodiment, a plurality of biomarkers from Table 4 are used in assessing apoptosis or a particular apoptosis pathway in which the biomarkers correspond to apoptotic cleavage of multiple protein substrates of a single apoptotic protease (e.g., caspase) of interest. In other embodiments, the biomarkers from Table 4 are selected so as to include biomarkers for the activity of a plurality of apoptotic proteases of interest.

In certain embodiments, a proteolytic apoptotic polypeptide biomarker of the invention may further comprise a recombinant sequence N-terminal or C-terminal to a sequence found in Table 4. For example, a biomarker of the invention may further comprise a fusion tag used to facilitate purification, detection, or both purification and detection of the polypeptide. Many fusion tags suitable for use with the present invention are well known in the art and include without limitation, polyhistidine tags, GST tags, biotin, calmodulin binding protein tags, chitin binding protein tags, TAP tags, Strep tags, Myc tags, HA tags, and the like. Other suitable recombinant sequences may further comprise a linker between the fusion tag and the polypeptide. Linker sequences may comprise a protease recognition site, such as a TEV cleavage site.

The present invention also provides proteolytic apoptotic cleavage junctions. In certain embodiments, a cleavage junction of the present invention may comprise an amino acid sequence targeted by a protease in response to an apoptotic stimulus. In a particular embodiment, the cleavage junctions of the present invention comprise sequences selected from those found in Table 4. In one embodiment, a cleavage junction of the invention comprises a full length protein containing a sequence identical to a sequence listed in Table 4. In a second embodiment, a cleavage junction of the present invention may comprise a protein fragment containing a sequence found in Table 4 that is competent for cleavage by a protease involved in apoptosis. In certain embodiments, the protein fragment may comprise about 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, 400, 500, 600, 700, 800, 900, 1000, or more amino acids of a protein identified by a Swiss-Prot ID found in Table 4. In some embodiments, the peptide is preferably at least about 6 amino acids long, 7 amino acids long, 8 amino acids long, 10 amino acids long and less than 50 amino acids long and can comprise or consist of an amino acid sequence (previous amino acid or C-terminal amino acid sequence, unmodified or identified amino acid, or N-terminal amino acid sequence, modified identified amino acid sequence, or protein) of Table 4. Preferred peptides for measuring the activity of apoptotic protease include the cleavage junction corresponding to a previous amino acid sequence of Table 4 and its corresponding immediately following identified or unmodified peptide of Table 4. A preferred range of peptide lengths is from about 7 to 50 amino acids in length and may include the full sequences of both the previous and identified or unmodified polypeptides of Table 4. Other suitable lengths range from 7 to 25, 7 to 15, 10 to 30, 15 to 35, and 15 to 25.

The apoptotic biomarkers of the present invention find use in the detection and quantification of apoptosis in a biological sample. In certain embodiments, the biomarkers can be used to detect apoptosis in a sample from an organism suffering from a disease characterized by apoptosis. In one embodiment, the biomarkers of the present invention can be used to diagnose or provide a prognosis for a disease characterized by apoptosis in an individual. In other embodiments the biomarkers can be used to determine the extent of apoptosis or the extent of a disease state in an individual or in a sample from an individual. In yet other embodiments, the biomarkers of the present invention are useful for determining the efficacy of a drug or for monitoring treatment in a patient. The biomarkers are particularly useful for determining the efficacy of drugs that induce apoptosis or for monitoring a treatment in a patient that results in apoptosis.

In one embodiment, the present invention provides proteolytic apoptotic signatures or profiles. In a specific embodiment, the apoptotic signatures of the present invention comprise one or more proteolytic polypeptide that is generated in response to an apoptotic stimulus. In another embodiment, an apoptotic signature of the invention comprises the level of at least one proteolytic apoptotic polypeptide biomarker in a biological sample. In one specific embodiment of the invention, an apoptotic signature comprises the level of at least one, preferably at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, or more proteolytic apoptotic polypeptides comprising an N-terminus or C-terminus selected from those found in Table 4, in a biological sample. In some embodiments, the N-terminus or C-terminus is that formed by the cleavage of a polypeptide by an apoptotic protease. In another embodiment of the invention, an apoptotic signature or profile comprises a plurality, or the level of a plurality, of proteolytic apoptotic cleavage junctions. In a specific embodiment, an apoptotic signature comprises the level of at least one, preferably at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, or more proteolytic apoptotic cleavage junctions, in a biological sample, selected from those found in Table 4. In further embodiments of the present invention, a proteolytic apoptotic signature may comprise a mixture of proteolytic apoptotic polypeptides and proteolytic apoptotic cleavage junctions, or the levels thereof, in a biological sample. In yet another embodiment, a proteolytic apoptotic signature comprises one or more ratio of a proteolytic apoptotic polypeptide to its corresponding intact proteolytic apoptotic cleavage junction in a biological sample. For example, a proteolytic apoptotic signature of the present invention may comprise at least one, preferably at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, or more ratios of cut to uncut proteolytic apoptotic cleavage junctions selected from those found in Table 4, or corresponding to the proteins identified by a Swiss-Prot ID found in Table 4, in a biological sample.

In certain embodiments, the present invention provides proteolytic apoptotic signatures that correspond to a specific level or degree of apoptosis in a biological sample, or in an individual. In other embodiments, the proteolytic apoptotic signatures of the present invention correspond to the level of apoptosis in a mammal suffering from a disease characterized by apoptosis. In yet other embodiments of the invention, a proteolytic apoptotic signature may correspond to a specific disease state or to a specific prognosis for a disease in an individual suffering with a disease characterized by apoptosis. In further embodiments, the proteolytic apoptotic signatures of the present invention may correspond to a specific efficacy for a drug administered to an individual or to a predicted response to a drug administered to an individual. The proteolytic apoptotic signatures of the present invention may be derived from a single biological sample from an individual or from a plurality of samples taken a group of individuals suffering from a disease characterized by apoptosis. In certain embodiments, the apoptotic signature may comprise an average of apoptotic signatures determined from a study or disease cohort.

In some embodiments, the present invention provides apoptotic signatures that correspond to healthy subjects, i.e. individuals that are not suffering from a disease, individuals that are suffering from a disease, individuals that have undergone therapy for a specific disease, individuals that have a good prognosis, individuals that have a bad prognosis, individuals with cancer, individuals with a high likelihood of developing metastatic cancer, individuals with a particular disease state, i.e. stage of cancer, severity of disease, benign tumor, and the like. As such, the various apoptotic signatures of the present invention find use in the diagnosis and prognosis of various diseases and disease states, as well as for monitoring the progression of a disease or the progression of a disease treatment regime.

In some embodiments, the invention provides synthetic peptides or polypeptides which are labeled with heavy isotopes of C, N, H, or O. For instance, ¹³C or ¹⁵N labeled peptides can be used as internal standards in the assay methods as known to one of ordinary skill in the art. By adding a known quantity of a heavy isotope-labeled peptide to a sample and then calculating the amount of the labeled polypeptide detected, it is possible to estimate the concentration of an unlabeled endogenous corresponding polypeptide in a sample by use of an analytical technique such as mass spectrometry (see, PCT Patent Publications WO 03026861 and WO 2008/054597), and see also, Carr et al., Clinical Chemistry 54:11 1749-1752 (2008) the contents of each of which are incorporated herein by reference in their entirety with respect to methods of quantitating proteins or polypeptides in a biological sample. Anderson et al., Journal of Proteome Research 2004, 3, 235-244; Carr et al., Nature Biotechnology 24(8):971 (2006); and Addona et al., Nature Biotechnology 27(7): 633 (2009); and McIntosh et al. Nature Biotechnology 27(7):622 (2009) are also each incorporated by reference in their entirety with respect to their disclosures of methods for detecting biomarkers in biological samples by targeted mass spectrometry. For instance, detection methods using selected reaction monitoring (SRM) or multiple reaction monitoring (MRM are contemplated.

An isotopically labeled peptide is preferably at least about 6 amino acids long, 7 amino acids long, 8 amino acids long, 10 amino acids long and less than 50 amino acids long and can comprise an amino acid sequence (previous amino acid or C-terminal amino acid sequence, identified or unmodified amino acid or N-terminal amino acid sequence, or the modified identified amino acid sequence of Table 4. Preferred labeled peptides for measuring the activity of apoptotic protease comprise a previous amino acid sequence of Table 4 with its corresponding immediately following identified or unmodified peptide of Table 4. A preferred ranged of labeled peptide lengths is from about 7 to 50 amino acids in length and may include the full sequences of both the previous and identified or unmodified polypeptides of Table 4.

The method detects and quantifies a target protein in a sample by introducing a known quantity of at least one heavy-isotope labeled peptide standard into a digested biological sample. By comparing to the peptide standard, one may readily determine the quantity of a peptide having the same sequence and protein modification(s) in the biological sample. Briefly, the methodology has two stages: (1) peptide internal standard selection and validation; method development; and (2) implementation using validated peptide internal standards to detect and quantify a target protein in a sample. The method is a powerful technique for detecting and quantifying a given peptide/protein within a complex biological mixture, such as a biological sample, a cell lysate, tissue section, or serum and may be used, e.g., to quantify change in protein as a result of drug treatment, or to quantify a protein in different biological states.

Generally, to develop a suitable internal standard, a particular peptide (or modified peptide) within a target protein sequence is chosen based on its amino acid sequence and a particular protease for digestion. The peptide can then be generated by solid-phase peptide synthesis such that one residue is replaced with that same residue containing stable isotopes (e.g., ¹³C, ¹⁵N). The result is a peptide that is chemically identical to its native counterpart formed by proteolysis, but is easily distinguishable by MS via a mass shift. A newly synthesized internal standard peptide is then evaluated by the detection method. This process provides qualitative information about peptide retention by the detection method.

The second stage of the strategy is its implementation to measure the amount of a protein or the modified form of the protein from complex mixtures. A biological sample such as a cell lysate, tissue section lysate, or serum may be extensively digested with a protease such as trypsin. Labeled peptides can then be spiked in to the complex peptide mixture obtained by digestion of the biological sample with a proteolytic enzyme, either before or after an optional affinity purification of a subset of the peptides in the mixture, as described above. The retention time and fragmentation pattern of the native peptide formed by digestion (e.g., trypsinization) is identical to that 25 of the labeled internal standard peptide determined previously; thus, the use of isotopically labeled peptides results in the highly specific and sensitive measurement of both internal standard and analyte directly from extremely complex peptide mixtures. Because an absolute amount of the labeled peptide is added, the ratio of the amount of endogenous peptide detected to the amount of labeled peptide detected can be used to determine the precise levels of a polypeptide, or more specifically, a proteolytic apoptotic polypeptide, in a sample.

In addition, the internal or labeled polypeptide standard when present during digestion and chromatography, such that peptide extraction efficiencies and absolute losses during sample handling (including vacuum centrifugation), and variability during introduction into the detection system do not affect the determined ratio of native and labeled polypeptide abundances.

A peptide sequence within a target protein is selected according to one or more criteria to optimize the use of the peptide as an internal standard. Preferably, the size of the peptide is selected to minimize the chances that the peptide sequence will be repeated elsewhere in other non-target proteins. Thus, a peptide is preferably at least about 6 amino acids. The size of the peptide is also optimized to maximize ionization frequency. Thus, peptides longer than about 20 amino acids are not preferred. The preferred ranged is about 7 to 15 amino acids. A peptide sequence is also selected that is not likely to be chemically reactive during mass spectrometry, thus sequences comprising cysteine, tryptophan, or methionine are avoided.

The peptide is labeled using one or more labeled amino acids (i.e. the label is an actual part of the peptide) or less preferably, labels may be attached after synthesis according to standard methods. Preferably, the label is a mass-altering label selected based on the following considerations: The mass should be unique to shift fragment masses produced by MS analysis to regions of the spectrum with low background; the ion mass signature component is the portion of the labeling moiety that preferably exhibits a unique ion mass signature in MS analysis; the sum of the masses of the constituent atoms of the label is preferably uniquely different than the fragments of all the possible amino acids. As a result, the labeled amino acids and peptides are readily distinguished from unlabeled ones by the ion/mass pattern in the resulting mass spectrum. Preferably, the ion mass signature component imparts a mass to a protein fragment that does not match the residue mass for any of the 20 natural amino acids.

The label should be robust under the fragmentation conditions of MS and not undergo unfavorable fragmentation. Labeling chemistry should be efficient under a range of conditions, particularly denaturing conditions, and the labeled tag preferably remains soluble in the MS buffer system of choice. The label preferably does not suppress the ionization efficiency of the protein and is not chemically reactive. The label may contain a mixture of two or more isotopically distinct species to generate a unique mass spectrometric pattern at each labeled fragment position. Stable isotopes, such as ¹³C, ¹⁵N, ¹⁷O, ¹⁸O, or ³⁴S, are among preferred labels. Pairs of peptide internal standards that incorporate a different isotope label may also be prepared.

Peptide internal standards are characterized according to their mass-to-charge (m/z) ratio, and preferably, also according to their behavior in chromatographic columns (e.g. an HPLC column) Internal standards that co-elute with unlabeled peptides of identical sequence are selected as optimal internal standards. The internal standard can then analyzed be fragmenting the peptide by any suitable means, for example by collision-induced dissociation (CID) using, e.g., argon or helium as a collision gas. The fragments can then be analyzed, for example, by multi-stage mass spectrometry (MSⁿ) to obtain a fragment ion spectrum, to obtain a peptide fragmentation signature. Preferably, peptide fragments have significant differences in m/z ratios to enable peaks corresponding to each fragment to be well separated, and a signature that is unique for the target peptide is obtained. If a suitable fragment signature is not obtained at the first stage, additional stages of MS are performed until a unique signature is obtained.

Fragment ions in the MS/MS and MS spectra are typically highly specific for the peptide of interest, and, in conjunction with LC methods, allow a highly selective means of detecting and quantifying a target peptide/protein in a complex protein mixture, such as a cell lysate, containing many thousands or tens of thousands of proteins. Any biological sample potentially containing a target protein/peptide of interest may be assayed. Crude or partially purified cell extracts are preferably used. Generally, the sample may have at least 0.01 mg of protein, typically a concentration of 0.1-10 mg/mL, and may be adjusted to a desired buffer concentration and pH.

Accordingly, internal peptide standards (heavy-isotope or light isotope labeled peptides) may be produced, as described above, for any of the novel polypeptides of the invention (see Table 4). These peptides may then be further used in assessing apoptotic enzyme activities in samples as described herein.

Quantitation of Corresponding Peptides Derived from the Neo-Epitopes in Samples.

In one embodiment, the present invention provides reagents for detecting the proteolytic apoptotic polypeptide biomarkers of the invention. In one embodiment, the reagents comprise proteins that bind to the biomarkers with high affinity and specificity. In another embodiment, the invention provides binding agents for detecting proteolytic apoptotic cleavage junctions. In a particular embodiment, the reagents comprise antibodies, or fragments thereof, generated against the proteolytic apoptotic polypeptides or proteolytic apoptotic cleavage junctions of the present invention. Suitable antibody fragment types include without limitation, F(ab′)2, F(ab), Fv, scFv, and the like. Antibodies can be generated by a number of well known methods including, without limitation, animal immunization, molecular display techniques, including phage display and ribosomal or mRNA display, rational design, and the like. In certain embodiments of the present invention, the binding agents further comprise a detectable moiety and/or a tag to facilitate purification of the binding reagent or binding reagent-biomarker complex.

In another embodiment, the present invention provides methods for generating binding reagents to one or more apoptotic biomarkers. In certain embodiments the apoptotic biomarkers comprise N-terminal or C-terminal sequences selected from those found in Table 4. In other embodiments, the apoptotic biomarkers comprise cleavage junctions selected from those found in Table 4. In a specific embodiment, the methods of the present invention comprise the steps of: (a) generating a plurality of proteolytic apoptotic polypeptides; (b) generating one or more binding reagents to said plurality of proteolytic apoptotic polypeptides; and (c) purifying at least one of said binding reagents. Pluralities of proteolytic apoptotic polypeptides can be generated, for example, by heterologous gene expression, in vitro translation, synthetic peptide synthesis, purification of proteolytic polypeptides from a biological sample, or in vitro proteolysis of peptides containing a proteolytic apoptosis cleavage junction. In one embodiment, the binding reagents comprise proteins or antibodies that specifically bind to either a proteolytic apoptotic polypeptide or to an intact cleavage junction corresponding to a proteolytic apoptotic polypeptide, but do not substantially bind to both.

In certain embodiments, the methods of the present invention for generating one or more antibodies comprise the steps of
(a) simultaneously immunizing a mammal with a plurality of apoptotic proteolytic polypeptides;
(b) collecting the immune serum from said mammal;
(c) affinity purifying a first antibody to a first proteolytic polypeptide,
(d) affinity purifying at least a second antibody to at least a second proteolytic polypeptide from the supernatant of step (c),
(e) removing antibodies that bind to the cleavage junction corresponding to said first proteolytic polypeptide by affinity means from said first antibody purification, and
(f) removing antibodies that bind to the cleavage junction corresponding to said at least second proteolytic polypeptide by affinity means from said second antibody purification, thereby generating at least two antibodies to proteolytic apoptotic polypeptides.
   These methods find use in generating a plurality of antibodies that bind to a proteolytic apoptotic polypeptide, but that do not substantially bind to the cleavage junction corresponding to said proteolytic polypeptide. In certain embodiments, the methods can be altered in order to generate a plurality of antibodies that bind to a proteolytic apoptotic cleavage junction, but that do not substantially bind to the corresponding proteolytic polypeptides generated in response to an apoptotic stimulus. In further embodiments, the methods of the present invention can be performed using molecular display techniques.

In yet other embodiments, the present invention provides methods of generating an antibody to the N-terminus or C-terminus of a proteolytic polypeptide, the method comprising the steps of: (a) Generating the N-terminal or C-terminal apoptotic product, by means of heterologous gene expression, in vitro transcription-translation, or synthetic methods, or by producing the full length protein and cleaving it with a protease to generate the N-terminal and C-terminal pieces and purification of the N-terminal proteolytic fragment, C-terminal proteolytic fragment, or any combination thereof; (b) using the N-terminal or C-terminal apoptotic fragment to generate one or more antibodies, either by immunization of animal, or in vitro selection methods such as phage display, ribosome display or other suitable display or selection methods, or to generate other suitable binding protein or proteins, either by in vitro selection methods such as phage display, ribosome display or other suitable display or selection methods

The present invention also provides methods of detecting proteolytic apoptotic biomarkers, including both proteolytic apoptotic polypeptides and proteolytic apoptotic cleavage junctions, in a biological sample. In one embodiment, the method comprises contacting a biological sample with a binding reagent that specifically binds to a proteolytic apoptotic biomarker of the present invention and detecting the binding reagent, thereby detecting the biomarker. In a second embodiment, the present invention provides methods of quantitating the amount of a proteolytic apoptotic biomarker in a biological sample, the method comprising the steps of contacting a biological sample with a binding reagent of the present invention, and determining the amount of biomarker is said sample. Methods of detecting and quantitating the amount of a polypeptide in a sample are well known in the art and include, without limitation, ELISA, immunohistochemical techniques, mass spectrometry, Luminex® xMAP technology, and the like.

In another embodiment, the present invention provides methods of detecting apoptosis in an individual. In one embodiment, the methods comprise detecting at least one proteolytic apoptotic polypeptide in a biological sample from an individual. In another specific embodiment, the methods comprise detecting an increased ratio of the level of at least a first proteolytic apoptotic polypeptide biomarker to the level of at least one first proteolytic apoptotic cleavage junction biomarker that corresponds to said first proteolytic apoptotic polypeptide. In some embodiments, the present methods comprise the detection or quantitation of at least about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, or more proteolytic apoptotic biomarkers of the present invention, or corresponding ratios thereof. In a second embodiment, the methods of the present invention comprise the detection of a proteolytic apoptotic signature in a biological sample from an individual, thereby detecting the presence of apoptosis is said individual. In yet another embodiment, the present invention provides methods of detecting a proteolytic apoptotic signature in a subject, the methods comprising the steps of: (a) determining the level of at least two proteolytic polypeptides in a biological sample from said subject; and (b) comparing said levels of at least two proteolytic polypeptides to a proteolytic apoptotic signature, thereby detecting a proteolytic signature in the subject, wherein said at least two proteolytic polypeptides comprise N-terminal or C-terminal sequences selected from those found in Table 4.

In one embodiment, the present invention provides methods of determining the level of apoptosis in an individual. In a particular embodiment, the methods comprise the steps of: (a) determining the level of at least one proteolytic polypeptide that is generated in response to an apoptotic stimulus in a biological sample from said subject; and (b) comparing said level of at least one proteolytic polypeptide to a biological signature corresponding to no apoptosis, thereby determining the level of apoptosis in the subject, wherein said at least one proteolytic polypeptide comprises an N-terminal or C-terminal sequence selected from those found in Table 4. In a related embodiment, the method further comprises the step of (c) comparing said level of at least one proteolytic polypeptide to at least one biological signature corresponding to a predetermined level of apoptosis. In a second embodiment, the methods comprise the steps of: (a) determining the level of at least one intact proteolytic apoptotic cleavage junction in a biological sample from said subject; (b) determining the level of at least one of the N-terminal or C-terminal proteolytic polypeptides corresponding to said at least one intact proteolytic apoptotic cleavage junction in said biological sample; and (c) determining the ratio of proteolytic polypeptides to intact proteolytic apoptotic cleavage junctions in said biological sample, thereby determining the level of apoptosis in the subject, wherein said proteolytic polypeptides are generated in response to an apoptotic stimulus.

In another embodiment, the invention provides methods of diagnosing or providing a prognosis for a disease characterized by apoptosis in an individual. In a specific embodiment, the methods comprise the steps of: (a) detecting a first proteolytic apoptotic signature in a biological sample from said individual; and (b) comparing said first proteolytic apoptotic signature to at least a second proteolytic apoptotic signature corresponding to a diagnosis or prognosis for a disease characterized by apoptosis, thereby diagnosing or providing a prognosis for a disease characterized by apoptosis in said individual. In other embodiments, the methods further comprise the steps of: (c) comparing said first apoptotic signature to at least a third apoptotic signature corresponding to a diagnosis of no disease or a second prognosis for said disease; and (d) determining which apoptotic signature said first apoptotic most highly correlates to, thereby diagnosing or providing a prognosis for a disease characterized by apoptosis in said individual.

Many correlation methodologies may be employed for the comparison of both individual proteolytic apoptotic biomarker levels and proteolytic apoptotic signatures or profiles in the present invention. Non-limiting examples of these correlation methods include parametric and non-parametric methods as well as methodologies based on mutual information and non-linear approaches. Examples of parametric approaches include without limitation, Pearson correlation (or Pearson r, also referred to as linear or product-moment correlation) and cosine correlation. Non-limiting examples of non-parametric methods include Spearman's R (or rank-order) correlation, Kendall's Tau correlation, and the Gamma statistic. Each correlation methodology can be used to determine the level of correlation between the levels or ratios of individual biomarkers in the data set. The correlation of all biomarkers with all other biomarkers is most readily considered as a matrix. Using Pearson's correlation as a non-limiting example, the correlation coefficient r in the method is used as the indicator of the level of correlation. When other correlation methods are used, the correlation coefficient analogous to r may be used, along with the recognition of equivalent levels of correlation corresponding to r being at or about 0.25 to being at or about 0.5. The correlation coefficient may be selected as desired to reduce the number of correlated biomarkers to various numbers. In particular embodiments of the invention using r, the selected coefficient value may be of about 0.25 or higher, about 0.3 or higher, about 0.35 or higher, about 0.4 or higher, about 0.45 or higher, or about 0.5 or higher.

In another embodiment, the present invention provides methods of monitoring the progression of therapy for a disease in an individual. In certain embodiments, the methods comprise determining the level of a proteolytic apoptotic biomarker or an apoptotic signature at different time points in a sample from an individual undergoing therapy for a disease. In some embodiments, the method will comprise comparing the levels of biomarkers or signatures at different times during the course of a disease treatment. Typically, a disease that is characterized by increased apoptosis, such as auto-imune diseases, will result in a decrease in apoptosis, as measured by the levels of biomarkers or signatures in a biological sample from an individual, during the course of a successful treatment regime. Conversely, a disease that is characterized by decreased apoptosis, such as cancer, will typically result in increased apoptosis, as measured by the levels of biomarkers or signatures in a biological sample from an individual, during the course of a successful treatment regime. In this fashion a biological sample from a patient that is responding favorably to a treatment regime will show a change, either increase or decrease, in the level of apoptosis over time, as measured by the methods of the present invention. In a particular embodiment, the methods of the present invention are useful for monitoring the progression of cancer therapy in an individual. The methods of the invention are compatible with all types of cancer therapy including, without limitation, chemotherapy, hormone therapy, biologic therapy, radiation therapy, surgical therapy, and the like.

In one embodiment, the present invention provides methods of determining the efficacy of a drug. In a specific embodiment, the methods comprise the steps of: (a) determining the level of at least one proteolytic polypeptide generated in response to an apoptotic stimulus in a biological sample from a first subject receiving a dose of said drug; (b) determining the level of at least one proteolytic polypeptide generated in response to an apoptotic stimulus in a biological sample from a second subject not receiving a dose of said drug; and (c) comparing said first and said second levels of said at least one proteolytic polypeptide, thereby determining the efficacy of said drug, wherein said at least one proteolytic polypeptide comprises an N-terminal or C-terminal sequence selected from those found in Table 4. In yet other embodiments of the invention, the method comprises determining a proteolytic apoptotic signature and comparing said signature to a second proteolytic apoptotic signature corresponding to a specific level of apoptosis. Drugs particularly well suited for use with the above methods include both drugs that induce apoptosis and anti-apoptotic drugs.

Many pharmaceuticals are known to cause apoptosis in vivo including, without limitation, nonsteroidal anti-inflammatory drugs (NSAIDs) (Yamazaki et al., Journal of Pharmacology and Experimental Therapeutics 302(1): 18-25 (2002)) and chemotherapeutic drugs. Examples of NSAIDs include, without limitation, Salicylates (including Acetylsalicylic acid (Aspirin), Amoxiprin Benorylate/Benorilate, Choline magnesium salicylate, Diflunisal, Ethenzamide, Faislamine, Methyl salicylate, Magnesium salicylate, Salicyl salicylate, and Salicylamide), Arylalkanoic acids (including Diclofenac, Aceclofenac, Acemetacin, Alclofenac, Bromfenac, Etodolac, Indometacin, Nabumetone, Oxametacin, Proglumetacin, Sulindac, and Tolmetin), 2-Arylpropionic acids (profens) (including Ibuprofen, Alminoprofen, Benoxaprofen, Carprofen, Dexibuprofen, Dexketoprofen, Fenbufen, Fenoprofen, Flunoxaprofen, Flurbiprofen, Ibuproxam, Indoprofen, Ketoprofen, Ketorolac, Loxoprofen, Naproxen, Oxaprozin, Pirprofen, Suprofen, and Tiaprofenic acid), N-Arylanthranilic acids (fenamic acids) (including Mefenamic acid, Flufenamic acid, Meclofenamic acid, and Tolfenamic acid), Pyrazolidine derivatives (including Phenylbutazone, Ampyrone, Azapropazone, Clofezone, Kebuzone, Metamizole, Mofebutazone, Oxyphenbutazone, Phenazone, Phenylbutazone, and Sulfinpyrazone), Oxicams (including Piroxicam, Droxicam, Lornoxicam, Meloxicam, and Tenoxicam), COX-2 inhibitors (including Celecoxib, Etoricoxib, Lumiracoxib, Parecoxib, Rofecoxib, Valdecoxib), Sulphonanilides including Nimesulide, histone deacetylase inhibitors (including Trichostatin A, cyclic tetrapeptides, benzamides, electrophilic ketones, phenylbutyrate, valproic acid, SAHA (approved by the FDA in 2007 for leukemia therapy under the name Vorinostat), Belinostat/PXD101, MS275, LAQ824/LBH589, CI994, MGCD0103 (Beckers et al., Int. J. Cancer 121(5): 1138-48 (2007)) nicotinamide, dihydrocoumarin, naphthopyranone, 2-hydroxynaphaldehydes, and the like). While not all NSAIDs induce apoptosis, one of skill in the art will know which drugs are appropriate for use in the present invention. Drugs that do not induce apoptosis, including some NSAIDs and some chemotherapeutic agents, may be used in combination with other drugs that do induce apoptosis in certain embodiments of the present invention.

Examples of chemotherapeutic anti-cancer drugs include, without limitation, Aminopterin, Methotrexate, Pemetrexed, Raltitrexed, Cladribine, Clofarabine, Fludarabine, Mercaptopurine, Pentostatin, Thioguanine, Cytarabine, Decitabine, Fluorouracil/Capecitabine, Floxuridine, Gemcitabine, Sapacitabine, Chlorambucil, Chlormethine, Cyclophosphamide, Ifosfamide, Melphalan, Bendamustine, Trofosfamide, Uramustine, Carmustine, Fotemustine, Lomustine, Nimustine, Prednimustine, Ranimustine, Semustine, Streptozocin, Carboplatin, Cisplatin, Nedaplatin, Oxaliplatin, Triplatin tetranitrate, Satraplatin, Busulfan, Mannosulfan, Treosulfan, Procarbazine, Dacarbazine, Temozolomide, Carboquone, ThioTEPA, Triaziquone, Triethylenemelamine, Docetaxel, Larotaxel, Ortataxel, Paclitaxel, Tesetaxel, Vinblastine, Vincristine, Vinflunine, Vindesine, Vinorelbine, Ixabepilone, Aclarubicin, Daunorubicin, Doxorubicin, Epirubicin, Idarubicin, Amrubicin, Pirarubicin, Mitoxantrone, Pixantrone, Valrubicin, Zorubicin, Actinomycin, Bleomycin, Mitomycin, Plicamycin, Hydroxyurea, Camptothecin, Topotecan, Irinotecan, Rubitecan, Belotecan, Etoposide, Teniposide, Altretamine, Amsacrine, Bexarotene, Estramustine, Irofulven, Trabectedin, Cetuximab, Panitumumab, Trastuzumab, Rituximab, Tositumomab, Alemtuzumab, Bevacizumab, Edrecolomab, Gemtuzumab, Axitinib, Bosutinib, Cediranib, Dasatinib, Erlotinib, Gefitinib, Imatinib, Lapatinib, Lestaurtinib, Nilotinib, Semaxanib, Sorafenib, Sunitinib, Vandetanib, Alvocidib, Seliciclib, Aflibercept, Denileukin diftitox, Aminolevulinic acid, Efaproxiral, Methyl aminolevulinate, Porfimer sodium, Temoporfin, Verteporfin, Alitretinoin, Tretinoin, Anagrelide, Arsenic trioxide, Pegaspargase, Atrasentan, Bortezomib, Carmofur, Celecoxib, Demecolcine, Elesclomol, Elsamitrucin, Etoglucid, Lonidamine, Lucanthone, Masoprocol, Mitobronitol, Mitoguazone, Mitotane, Oblimersen, Omacetaxine, Sitimagene ceradenovec, Testolactone, Tiazofurine, Tipifarnib, and the like. While not all chemotherapeutic drugs induce apoptosis, one of skill in the art will know which drugs are appropriate for use in the present invention.

The invention also provides RNA interference, or RNAi, by use of siRNA or shRNA molecules directed toward a protein of Table 4. An “siRNA” or “shRNA” refers to a nucleic acid that forms a double stranded RNA, which double stranded RNA has the ability to reduce or inhibit expression of a gene or target gene when the siRNA expressed in the same cell as the gene or target gene. “siRNA” or “shRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 or about 24-29 nucleotides in length, e.g., 20, 21, 22, 23, 24, 25, 26, 27, 28, 29, or 30 nucleotides in length. The siRNA find use in moulating apoptosis, treating cancer by promoting apoptosis, and in treating conditions in which the modulation or promotion of apoptosis would be beneficial, or in treating disease or conditions characterized by apoptosis. The subjects are preferably human.

The design and making of siRNA molecules and vectors are well known to those of ordinary skill in the art. For instance, an efficient process for designing a suitable siRNA is to start at the AUG start codon of the mRNA transcript and scan for AA dinucleotide sequences (see, Elbashir et al. EMBO J 20: 6877-6888 (2001). Each AA and the 3′ adjacent nucleotides are potential siRNA target sites. The length of the adjacent site sequence will determine the length of the siRNA. For instance, 19 adjacent sites would give a 21 nucleotide long siRNA siRNAs with 3′ overhanging UU dinucleotides are often the most effective. This approach is also compatible with using RNA pol III to transcribe hairpin siRNAs. RNA pol III terminates transcription at 4-6 nucleotide poly(T) tracts to create RNA molecules having a short poly(U) tail. However, siRNAs with other 3′ terminal dinucleotide overhangs can also effectively induce RNAi and the sequence may be empirically selected. For selectivity, target sequences with more than 16-17 contiguous base pairs of homology to other coding sequences can be avoided by conducting a BLAST search (see, www.ncbi.nlm.nih.gov/BLAST).

The siRNA or shRNA can be administered directly or an siRNA or shRNA expression vector can be used to induce RNAi. A vector can have inserted two inverted repeats separated by a short spacer sequence and ending with a string of T's which serve to terminate transcription. The expressed RNA transcript is predicted to fold into a short hairpin shRNA. The selection of shRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary. A preferred order of the shRNA expression cassette is sense strand, short spacer, and antisense strand. shRNAs with these various stem lengths (e.g., to 30) are suitable. The length of the loops linking sense and antisense strands of the shRNA can have varying lengths (e.g., 3 to 9 nucleotides, or longer). The vectors may contain promoters and expression enhancers or other regulatory elements which are operably linked to the nucleotide sequence encoding the shRNA.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. These control elements may be designed to allow the clinician to turn off or on the expression of the gene by adding or controlling external factors to which the regulatory elements are responsive.

In yet another embodiment, the present invention provides kits for detecting or quantitating the biomarkers of the present invention. In certain embodiments, these kits comprise binding reagents, such as antibodies or proteins, that specifically bind the biomarkers of the invention. In other embodiments, the kits of the present invention comprise protein binding arrays for the detection or quantitation of the biomarkers of the invention. In one embodiment, the kits of the present invention are useful in the detection or quantitation of apoptosis in a biological sample. In a second embodiment, the kits of the invention are useful for diagnosing or for providing a prognosis for a disease characterized by apoptosis in an individual.

The present invention also provides novel enzymatic approaches for positive selection of protein fragments containing unblocked α-amines, characteristically produced in proteolysis. This approach makes use of an engineered peptide ligase termed subtiligase to selectively biotinylate unblocked protein α-amines in complex samples with great selectivity over ε-amines of lysine side chains. Site-specific biotinylation permits subsequent purification and identification of corresponding N-terminal peptides using tandem mass spectrometry (LC/MS/MS).

Proteomic workflow utilizing subtiligase that enables biotinylation of protein α-amines in complex mixtures and subsequent cataloguing of N-termini in a given sample has been previously described (see US Pat. Pub. No. 2012-0028266 A1). Thus detergent lysates of either normal or apoptotic cells are first prepared in the presence of protease inhibitors to quench all protease activity. Proteins in these lysates are then N-terminally biotinylated by treatment with subtiligase and a peptide glycolate ester substrate specially tailored to the proteomic workflow. Biotinylated samples are then exhaustively digested with trypsin, and N-terminal peptides are captured using avidin affinity media. The peptide ester substrate contains a tobacco etch virus (TEV) protease cleavage site between biotin and the site of ligation to permit facile recovery of captured peptides (Rigaut et al., 1999). An important aspect of the workflow is that all labeled peptides recovered using TEV protease retain an N-terminal SY-dipeptide modification. This modification provides a key hallmark to distinguish ligated peptides from contaminating unligated ones using LC/MS/MS. Identification of recovered SY-peptides permits identification of corresponding proteins, native N-termini, and localization of proteolytic processing sites.

### Methods of Biomarker Detection

The methods provided herein are useful, inter alia, to detect biomarkers (e.g., peptides as provided herein) indicative of apoptosis in a cancer patient. In embodiments, the peptides are proteolytic peptides. In embodiments, the peptides are proteolytic apoptotic peptides. The proteolytic peptides as provided herein are peptides formed by proteolytic cleavage (proteolysis) performed by a cellular protease. In embodiments, the cellular protease is a calpain protease. In embodiments, the cellular protease is a serine protease. In embodiments, the cellular protease is a cysteine protease. In embodiments, the cellular protease is a cysteine-aspartic protease. In embodiments, the cellular protease is a caspase. In embodiments, the caspase is caspase-3, caspase-6, or caspase-7. The peptides detected herein may be derived from a cancer patient. In embodiments, the cancer patient receives or has received a therapeutic agent (e.g., a chemotherapeutic agent). In embodiments, the peptide is detected in serum derived from the cancer patient receiving a chemotherapeutic agent. In embodiments, the peptide detected in the serum of a cancer patient receiving a chemotherapeutic agent is absent in the serum of a non-cancer patient.

In one aspect, a method of determining a level of a peptide in a cancer subject is provided. The method includes (i) assaying a biological sample from a cancer subject; and (ii) determining a level of a peptide as set forth in Table 4 in the biological sample.

In another aspect, a method of determining a level of a peptide in a cancer subject is provided. The method includes (i) assaying a biological sample from a cancer subject; and (ii) determining a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in the biological sample. In embodiments, the determining includes determining a level of a plurality of peptides. In embodiments, the determining includes determining a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 and determining a level of a peptide of SEQ ID NO:1-401. In embodiments, the determining includes determining a level of a peptide as set forth in Table 4 and determining a level of a peptide of SEQ ID NO:1-401.

In embodiments, the determining includes contacting a binding reagent with the peptide to form a binding reagent peptide complex. In embodiments, the binding reagent is an antibody or an aptamer. In embodiments, the antibody is a monoclonal Ab or a functional fragment thereof. In embodiments, the binding reagent does not substantially bind to a corresponding full-length protein including SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:55.

In embodiments, the subject has a hematologic malignancy. In embodiments, the hematologic malignancy is multiple myeloma, acute myeloid leukemia or diffuse large B-cell lymphoma. In embodiments, the biological sample is a blood-derived biological sample or a tissue-derived biological sample of the subject. In embodiments, the subject is receiving or has received a therapeutic agent. In embodiments, the therapeutic agent is a chemotherapeutic agent, a radiotherapeutic agent, an apoptosis inducing agent or a cytotoxic agent. In embodiments, the chemotherapeutic agent is a proteasome inhibitor, a nucleoside analog or a DNA damaging agent.

In another aspect, a method of determining apoptosis in a subject is provided. The method includes (i) detecting a level of a peptide as set forth in Table 4 in a biological sample from a subject receiving or having received a therapeutic agent, wherein the detecting includes contacting a binding reagent to the peptide to form a binding reagent peptide complex and detecting the binding reagent peptide complex. The level is compared to a standard control, thereby determining apoptosis in a subject.

In another aspect, a method of determining apoptosis in a subject is provided. The method includes (i) detecting a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in a biological sample from a subject receiving or having received a therapeutic agent, wherein the detecting includes contacting a binding reagent with the peptide to form a binding reagent peptide complex and detecting the binding reagent peptide complex. The level is compared to a standard control, thereby determining apoptosis in a subject.

In embodiments, the binding reagent is an antibody or an aptamer. In embodiments, the antibody is a monoclonal Ab or a functional fragment thereof. In embodiments, the subject has cancer. In embodiments, the subject has a hematologic malignancy. In embodiments, the hematologic malignancy is multiple myeloma, acute myeloid leukemia or diffuse large B-cell lymphoma. In embodiments, the biological sample is a blood-derived biological sample or a tissue-derived biological sample of the subject. In embodiments, the therapeutic agent is a chemotherapeutic agent, a radiotherapeutic agent, an apoptosis inducing agent or a cytotoxic agent. In embodiments, the chemotherapeutic agent is a proteasome inhibitor, a nucleoside analog or a DNA damaging agent.

In another aspect, a method of determining efficacy of a therapeutic agent in a subject is provided. The method includes (i) detecting a level of a peptide as set forth in Table 4 in a sample from a subject receiving a therapeutic agent, wherein the detecting includes contacting a binding reagent with the peptide to form a binding reagent peptide complex and detecting the binding reagent peptide complex. (ii) It is determined whether the level is increased relative to a standard control, wherein an elevated level of a peptide relative to the standard control indicates efficacy of the therapeutic agent. And (iii) based at least in part on the level in step (ii), determining efficacy of the therapeutic agent.

In another aspect, a method of determining efficacy of a therapeutic agent in a subject is provided. The method includes (i) detecting a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in a sample from a subject receiving a therapeutic agent, wherein the detecting includes contacting a binding reagent with the peptide to form a binding reagent peptide complex and detecting the binding reagent peptide complex. (ii) It is determined whether the level is increased relative to a standard control, wherein an elevated level of a peptide relative to the standard control indicates efficacy of the therapeutic agent. And (iii) based at least in part on the level in step (ii), determining efficacy of the therapeutic agent. In embodiments, the binding reagent is an antibody or an aptamer. In embodiments, the antibody is a monoclonal Ab or a functional fragment thereof.

The term “peptides set forth in Table 4” as provided herein refers to the peptides of SEQ ID NO:439-591, respectively. Therefore, a person of ordinary skill in the art will immediately understand that the peptides of SEQ ID NO:439-591 are the peptides set forth in Table 4.

For the methods provided herein including embodiments thereof, determining a level of a peptide may include contacting a binding reagent with the peptide to form a binding reagent peptide complex. In embodiments, the method includes detecting the binding reagent peptide complex. In embodiments, the binding reagent is an antibody or an aptamer. In embodiments, the antibody is a monoclonal Ab or a functional fragment thereof. In embodiments, the antibody includes a detectable moiety (e.g., a fluorescent moiety, luminescent moiety, colorimetric moiety, phosphorescent moiety, radioactive moiety or electroactive moiety). In embodiments, the detectable moiety is non-covalently bound to a solid support. In embodiments, the binding reagent is bound to a solid support, wherein the solid support includes glass, plastic, ceramic, modified silica, nylon or quartz. In embodiments, the binding reagent is non-covalently bound to a solid support. In embodiments, the peptide is bound to a detectable moiety. In embodiments, the binding reagent peptide complex is in a buffer. In embodiments, the buffer includes at least one non-naturally occurring component. A non-naturally occurring component is a component not found in nature. In embodiments, the non-naturally occurring component is tris(hydroxymethyl)aminomethane (Tris). In embodiments, the buffer has a non-naturally occurring pH. In embodiments, the buffer does not have a physiological pH. A physiological pH as provided herein is a pH of less or more than 7.4. In embodiments, the buffer has a pH of less than 7.4. In embodiments, the buffer has a pH of more than 7.4. In embodiments, the non-naturally occurring component is a surfactant. In embodiments, the surfactant is sodium dodecyl sulfate. In embodiments, the non-naturally occurring component is a redox reagent. In embodiments, the redox reagent is dithiothreitol.

In embodiments, the peptide is covalently bound to a detectable moiety (e.g., a fluorescent moiety, luminescent moiety, colorimetric moiety, phosphorescent moiety, radioactive moiety or electroactive moiety). In embodiments, the detectable moiety includes biotin. In embodiments, the detectable moiety is biotin. When the detectable moiety is an amino acid sequence, the combined sequence of peptide and detectable moiety is a non-naturally occurring sequence. In embodiments the detectable moiety is not an amino acid sequence although it can include an amino acid sequence. The detectable moiety may include a linker and the linker may be an amino acid sequence and the detectable moiety is not an amino acid sequence. In embodiments, linker is a covalent linker. In embodiments, the linker is non-covalent linker. In embodiments, the detectable moiety includes biotin and a tobacco etch virus (TEV) protease cleavage site. In embodiments, the detectable moiety includes a cleaved tobacco etch virus (TEV) protease cleavage site.

### Compositions

In another aspect, an in vitro polypeptide complex is provided. The in vitro polypeptide complex includes a peptide bound to a binding reagent, wherein the peptide is SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552. In embodiments, the binding reagent is an antibody or an aptamer. In embodiments, the antibody is a monoclonal Ab or a functional fragment thereof. In embodiments, the antibody includes a detectable moiety (e.g., a fluorescent moiety, luminescent moiety, colorimetric moiety, phosphorescent moiety, radioactive moiety or electroactive moiety). In embodiments, the binding reagent is bound to a solid support, wherein the solid support includes glass, plastic, ceramic, modified silica, nylon or quartz. In embodiments, the peptide is covalently bound to a detectable moiety.

In another aspect, a conjugate is provided. The conjugate includes a peptide covalently bound to a detectable moiety, wherein the peptide is SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552. In embodiments, the peptide is bound to a binding reagent. In embodiments, the binding reagent is an antibody or an aptamer. In embodiments, the antibody is a mAb or a functional fragment thereof. In embodiments, the detectable moiety is attached to a solid support. In embodiments, the solid support includes glass, plastic, ceramic, modified silica, nylon or quartz.

In another aspect, an antibody or aptamer that specifically binds to SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 is provided.

### DEFINITIONS

“Subtiligase” refers generally to proteins which have the enzymatic activity of being able to ligate esterified peptides site-specifically onto the N termini of proteins or peptides. An example of such a subtiligase is one derived from the enzyme subtilisin BPN′ by site directed mutagenesis to effect the double substitution Ser221Cys and Pro225Ala, as described herein. Also described herein are additional subtiligases which have been engineered to exhibit other advantageous features, such as enhanced stability.

A “substrate” used in the context of subtiligase refers generally to any chemical moiety that is capable of being utilized during the enzymatic action of subtiligase that results in the specific labeling of the N termini of proteins or peptides by subtiligase. Examples of such substrates include peptide esters as described in greater detail herein.

“A complex mixture” refers generally to any composition that is composed of at least two or more proteins or peptides containing α-amines. A complex mixture can have at least two different proteins encoded by different genes; a complex mixture can be naturally occurring (e.g., a cell extract) or prepared (e.g., a formulation); a complex mixture can have recombinant, synthetic, or naturally occurring proteins or a mixture thereof. In many cases, a complex sample is one which displays a high degree of heterogeneity of proteins or peptides. Examples of complex mixtures include whole cells, cell extracts, partially purified cell extracts, tissues, bodily fluids, and animals, among others. Accordingly, in some embodiments, such complex mixtures comprise the naturally occurring proteins found in cells and tissues encoded by, for instance, different genes as found in the genomes of the source of the complex mixture (e.g., a cell or tissue extract or a bodily fluid such as serum). However, a complex mixture can also contain, as a component thereof, a recombinant protein or a purified protein or polypeptide either as an endogenous component (in the case of a recombinant protein), or as one added exogenously to the composition.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “cleavable linker” when used in the context of a peptide ester of the present invention refers generally to any element contained within the peptide that can serve as a spacer and is labile to cleavage upon suitable manipulation. Accordingly, a cleavable linker may comprise any of a number of chemical entities, including amino acids, nucleic acids, or small molecules, among others. A cleavable linker may be cleaved by, for instance, chemical, enzymatic, or physical means. Non-limiting examples of cleavable linkers include protease cleavage sites and nucleic acid sequences cleaved by nucleases. Further, a nucleic acid sequence may form a cleavable linker between multiple entities in double stranded form by complementary sequence hybridization, with cleavage effected by, for instance, application of a suitable temperature increase to disrupt hybridization of complementary strands. Examples of chemical cleavage sites include the incorporation photolabile, acid-labile, or base-labile functional groups into peptides.

“Proteases” (or “proteinases”, “peptidases”, or “proteolytic” enzymes) generally refer to a class of enzymes that cleave peptide bonds between amino acids of proteins. Because proteases use a molecule of water to effect hydrolysis of peptide bonds, these enzymes can also be classified as hydrolases. Six classes of proteases are presently known: serine proteases, threonine proteases, cysteine proteases, aspartic acid proteases, metalloproteases, and glutamic acid proteases (see, e.g., Barrett A. J. et al. The Handbook of Proteolytic Enzymes, 2nd ed. Academic Press, 2003).

Proteases are involved in a multitude of physiological reactions from simple digestion of food proteins to highly regulated cascades (e.g., the cell cycle, the blood clotting cascade, the complement system, and apoptosis pathways). It is well known to the skilled artisan that proteases can break either specific peptide bonds, depending on the amino acid sequence of a protein, or break down a polypeptide to constituent amino acids.

Among the proteases of this invention are “caspases”, a family of cysteine proteases, which cleave other proteins after an aspartic acid residue. Many of the caspases are held in an inactive form as a zymogen until they are activated by proteolytic cleavage, which converts the inactive caspase into an active conformation, allowing caspase cleavage of downstream targets. Caspases serve an essential role in apoptosis, in which a cascade of sequential caspase activation is responsible executing programmed cell death. See, e.g., Thornberry, N. L. and Lazebnik, Y., Science, 281:1312-1316 (1998); Shi, Y., Cell, 117:855-8 (2004) for reviews. As an example of this regulatory hierarchy, caspase-3 is processed into an active form through its proteolysis by caspases-8, -9, and -10. Upon activation, caspase 3 is then able to activate caspases-6 and -7 via proteolysis. Caspases-3, -6, and -7 are then able to proteolyze cellular substrates such as nuclear lamins. Caspases can also become inappropriately and acutely activated during stroke, myocardial infarction, or Parkinson's disease.

“Apoptosis” refers generally to a process of programmed cell death and involves a series of ordered molecular events leading to characteristic changes in cell morphology and death, as distinguished from general cell death or necrosis that results from exposure of cells to non-specific toxic events such as metabolic poisons or ischemia. Cells undergoing apoptosis show characteristic morphological changes such as chromatin condensation and fragmentation and breakdown of the nuclear envelope. As apoptosis proceeds, the plasma membrane is seen to form blebbings, and the apoptotic cells are either phagocytosed or else break up into smaller vesicles which are then phagocytosed. Typical assays used to detect and measure apoptosis include microscopic examination of cellular morphology, TUNEL assays for DNA fragmentation, caspase activity assays, annexin-V externalization assays, and DNA laddering assays, among others. It is well known to the skilled artisan that the process of apoptosis is controlled by a diversity of cell signals which includes extracellular signals such as hormones, growth factors, cytokines, and nitric oxide, among others. These signals may positively or negatively induce apoptosis. Other effectors of apoptosis include oncogenes (e.g., c-myc) and exposure of cancer cells to chemotherapeutic agents, among other examples.

“Inducing apoptosis” or “inducer of apoptosis” refers to an agent or process which causes a cell to undergo the program of cell death described above for apoptosis.

A “cell signal” refers to any agent which may initiate or stimulate directly or indirectly proteolysis within a cell. Examples of cell signals include agents that cause cells to undergo apoptosis such as those discussed above. In the context of this invention, a cell signal may include introduction of an activated or overexpressed oncogene, such as c-myc, or any other protein that causes a proteolytic event to occur within cells, as well as, externally applied agents (e.g., chemotherapeutic drugs, etc.).

A “peptide ester” refers generally to any peptide in which one carboxyl group of the peptide is esterified, i.e., is of the structure —CO—O—R. In embodiments of this invention, a peptide ester can serve as a substrate for subtiligase such that the peptide is added to the α-amino group of polypeptides to form the structure —CO—NH—R, thus labeling the polypeptide. In some embodiments of this invention, a peptide ester can carry a detectable label and a site for proteolysis or another form of chemical cleavage (e.g., through introduction of photolabile, acid-labile, or base-labile functional groups).

A “detectable moiety”, “label” or “detectable label” or “tag” is a composition detectable by mass spectrometric, spectroscopic, photochemical, biochemical, immunochemical, or chemical means. For example, useful labels include radioactive isotopes (e.g., 3H, 35S, 32P, 51Cr, or 125I), stable isotopes (e.g., ¹³C or ¹⁵N), fluorescent dyes, electron-dense reagents, enzymes (e.g., alkaline phosphatase, horseradish peroxidase, or others commonly used in an ELISA), biotin, digoxigenin, or haptens or epitopes and proteins for which antisera or monoclonal antibodies are available. Any appropriate method known in the art for conjugating an antibody to the label may be employed, e.g., using methods described in Hermanson, Bioconjugate Techniques 1996, Academic Press, Inc., San Diego. In general, a label as used in the context of the present invention is any entity that may be used to detect or isolate the product of the subtiligase ligation reaction. Thus, any entity that is capable of binding to another entity maybe used in the practice of this invention, including without limitation, epitopes for antibodies, ligands for receptors, and nucleic acids, which may interact with a second entity through means such as complementary base pair hybridization.

A “labeled protein or peptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

A “solid support” as provided herein refers to any solid material that can be attached or associated with, for example, a binding reagent or peptide as provide herein including embodiments thereof and is amenable to the methods provided herein including embodiments thereof. Examples of solid supports include without limitation, glass and modified or functionalized glass (e.g., carboxymethyldextran functionalized glass), plastics (including acrylics, polystyrene and copolymers of styrene and other materials, polypropylene, polyethylene, polybutylene, polyurethanes, Teflon™, etc.), polysaccharides, nylon or nitrocellulose, composite materials, ceramics, and plastic resins, silica or silica-based materials including silicon and modified silicon (e.g., patterned silicon), carbon, metals, quartz (e.g., patterned quartz), inorganic glasses, plastics, optical fiber bundles, and a variety of other polymers. In general, the substrates allow optical detection and do not appreciably fluoresce.

The term “aptamer” as provided herein refers to short oligonucleotides (e.g. deoxyribonucleotides), which fold into diverse and intricate molecular structures that bind with high affinity and specificity to proteins, peptides, and small molecules in a non-Watson Crick manner. An aptamer can thus be used to detect or otherwise target nearly any molecule of interest, including a fibrotic pulmonary disease marker protein. Methods of constructing and determining the binding characteristics of aptamers are well known in the art. For example, such techniques are described in U.S. Pat. Nos. 5,582,981, 5,595,877 and 5,637,459. Aptamers are typically at least 5 nucleotides, 10, 20, 30 or 40 nucleotides in length, and can be composed of modified nucleic acids to improve stability. Flanking sequences can be added for structural stability, e.g., to form 3-dimensional structures in the aptamer. Aptamers can be selected in vitro from very large libraries of randomized sequences by the process of systemic evolution of ligands by exponential enrichment (SELEX as described in Ellington A D, Szostak J W (1990) In vitro selection of RNA molecules that bind specific ligands. Nature 346:818-822; Tuerk C, Gold L (1990) Systematic evolution of ligands by exponential enrichment: RNA ligands to bacteriophage T4 DNA polymerase. Science 249:505-510) or by developing SOMAmers (slow off-rate modified aptamers) (Gold L et al. (2010) Aptamer-based multiplexed proteomic technology for biomarker discovery. PLoS ONE 5(12):e15004). Applying the SELEX and the SOMAmer technology includes for instance adding functional groups that mimic amino acid side chains to expand the aptamer's chemical diversity. As a result high affinity aptamers for almost any protein target are enriched and identified.

A “biomarker” or “biomarker peptide” as provided herein refers to any assayable characteristics or compositions that is used to identify, predict, or monitor a condition (e.g., a level of apoptosis). A biomarker is, for example, a peptide or combination of peptides whose presence, absence, or relative amount is used to identify a condition (e.g. level of apoptosis) or status of a condition (e.g. level of apoptosis) in a subject or sample. Biomarkers identified herein are measured to determine levels, expression, activity, or to detect fragments, variants or homologs of said biomarkers. Variants include amino acid or nucleic acid variants or post translationally modified variants. In embodiments, the biomarker is a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552. In embodiments, the biomarker is a peptide as set forth in Table 4. The biomarker peptides provided herein are identified by accession numbers referring to the corresponding amino acid and/or nucleic acid sequence of the individual biomarker peptides. Further, the biomarker peptides provided herein are referred to by sequence identifiers. Therefore, a person of ordinary skill in the art will immediately recognize the sequences of the biomarker peptides provided herein.

In some examples of the disclosed methods, when the level of a peptide(s) is assessed, the level is compared with control expression level of the peptide(s) (e.g., standard control). By control level is meant the expression level of a particular peptide(s) from a sample or subject lacking a disease (e.g. cancer), at a selected stage of a disease or disease state, or in the absence of a particular variable such as a therapeutic agent. Alternatively, the control level comprises a known amount of peptide. Such a known amount correlates with an average level of subjects lacking a disease, at a selected stage of a disease or disease state, or in the absence of a particular variable such as a therapeutic agent. A control level also includes the level of one or more peptides from one or more selected samples or subjects as described herein. For example, a control level includes an assessment of the level of one or more peptides in a sample from a subject that does not have a disease (e.g. cancer), is at a selected stage of a disease (e.g. cancer), or has not received treatment for a disease. Another exemplary control level includes an assessment of the level of one or more peptides in samples taken from multiple subjects that do not have a disease, are at a selected stage of a disease, or have not received treatment for a disease.

When the control level or standard control includes the level of one or more peptides in a sample or subject in the absence of a therapeutic agent (e.g., chemotherapeutic agent), the control sample or subject is optionally the same sample or subject to be tested before or after treatment with a therapeutic agent or is a selected sample or subject in the absence of the therapeutic agent. Alternatively, a control level is an average expression level calculated from a number of subjects without a particular disease. A control level or standard control also includes a known control level or value known in the art.

“Biological sample” as used herein is a sample of cells, biological tissue, or fluid that is to be tested for the occurrence of proteolysis or the presence, more generally, of polypeptides of interest in the sample. Among the cells that can be examined are cancer cells, cells stimulated to under apoptosis, and cells at different stages of development, among others. The biological tissues of this invention include any of the tissues that comprise the organs of an organism. The biological sample can be derived from any species including bacteria, yeasts, plants, invertebrates, and vertebrate organisms. The fluid of this invention can be any fluid associated with a cell or tissue. Such fluids may include the media in which cells are cultured as well as the fluid surrounding tissues and organs, as well as the fluid comprising the circulatory system of invertebrates and vertebrates (e.g., body fluids such as whole blood, serum, plasma, cerebrospinal fluid, urine, lymph fluids, and various external secretions of the respiratory, intestinal and genitourinary tracts, tears, saliva, milk, white blood cells, myelomas, and the like). An “extracellular fluid” refers generally to any fluid found exterior to cells. Such fluids may include all of the fluids described above. In certain embodiments, such fluids may further include cellular debri, for example from lysed cells, including membrane-bound and cytosolic proteins. A biological sample used in the present invention may be from a suitable organism, for example a mammal such as a mouse, rat, hamster, guinea pig, rabbit, sheep, goat, pig, monkey, human, and the like.

As used herein, the term “conjugate” refers to the association between atoms or molecules. The association can be direct or indirect. For example, a conjugate between a peptide and a detectable moiety can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In other embodiments, the peptide includes one or more reactive moieties, e.g., a covalent reactive moiety, as described herein (e.g., an amine reactive moiety). In other embodiments, the peptide includes a linker with one or more reactive moieties, e.g., a covalent reactive moiety, as described herein (e.g., an amine reactive moiety).

Useful reactive moieties or functional groups used for conjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding;

(m) metal bonding to reactive phosphorus groups (e.g. phosphines) to form, for example, phosphate diester bonds; and

(n) sulfones, for example, vinyl sulfone.

The reactive functional groups can be chosen such that they do not participate in, or interfere with, the chemical stability of the peptides or detectable moieties described herein.

A “negative control” has the definition recognized by the skilled artisan and generally refers to an experiment in which the desired result is no effect. Conversely, a “positive control” is a control experiment in which the desired outcome is a well-defined or well-known effect. In the context of this invention, a negative control may be a biological sample which is not treated with an agent that provides a cell signal to stimulate proteolysis or may be a sample treated with a placebo.

“Secreted protein” refers generally to any protein that is synthesized by a cell for export to the exterior of the cell membrane, for instance, secretion to the extracellular fluid. A variety of secreted proteins are recognized by the skilled artisan including: hormones, growth factors, antibiotics, antibodies, neuropeptides, toxins, cytokines, apolipoproteins, proteases and protease inhibitors, among others.

“Disease” or “disease state” refers generally to any derangement of normal physiology. Examples of diseases relevant to the practice of this invention include, without limitation: inflammatory diseases such as rheumatoid arthritis, osteoporosis, inflammatory bowel syndrome, asthma; cardiovascular diseases such as ischemia, stroke, myocardial infarction, congestive heart failure, atherosclerosis; type I and II diabetes and diabetes related diseases such as hyperglycemia, diabetic retinopathy, peripheral neuropathy; thrombotic disorders, such as diseases affecting blood clotting or complement fixation; neurodegenerative diseases such as Alzheimer's disease, Parkinson's disease, Huntington's disease, age-related dementia; liver diseases, such as liver infection, fibrosis, cirrhosis; kidney infection, fibrosis, and cirrhosis; muscular dystrophy; multiple sclerosis; lung diseases, such as lung fibrosis; schizophrenia and other mental disorders; and disorders of cell proliferation such as psoriasis and cancer (see below). (See, generally, Harrison's Principles of Internal Medicine, 16th edition, 2004.)

“Cancer” and “cancer cells” refers generally to human and animal cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas) and Hodgkin's lymphoma, leukemia (including AML, ALL, and CML), multiple myeloma, mantle cell lymphoma, Waldenstrom's macrogobulinemia, and Philadelphia positive cancers, among others.

“Chemotherapeutic drugs or agents” include conventional chemotherapeutic reagents such as alkylating agents, anti-metabolites, plant alkaloids, antibiotics, and miscellaneous compounds e.g., cis-platinum, CDDP, methotrexate, vincristine, adriamycin, bleomycin, and hydroxyurea, as well as biologics, such as therapeutic antibodies. Chemotherapeutic agents can include other therapeutic approaches known in the art for treating cancer, such as radiation therapy. Chemotherapeutic drugs or agents can be used alone or in combination in the practice of the present invention. The methods of the present invention are useful in combination with adjuvant cancer therapies, including hormone therapy, chemotherapy, biologic therapy (i.e. antibody therapy), radiation therapy, immunotherapy, surgery, and the like.

By “therapeutically effective amount or dose” or “sufficient amount or dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins)

“Metastasis” refers to spread of a cancer from the primary tumor or origin to other tissues and parts of the body, such as the lymph nodes.

“Providing a prognosis” refers to providing a prediction of the likelihood of metastasis, predictions of disease free and overall survival, the probable course and outcome of cancer therapy, or the likelihood of recovery from the cancer, in a subject.

“Diagnosis” refers to identification of a disease state, such as cancer, in a subject. The methods of diagnosis provided by the present invention can be combined with other methods of diagnosis well known in the art. Non-limiting examples of other methods of diagnosis include, detection of previously known disease biomarkers, including protein and nucleic acid biomarkers, radiography, co-axial tomography (CAT) scans, positron emission tomography (PET), radionuclide scanning, and the like.

The terms “cancer-associated antigen”, or “tumor-specific marker”, or “tumor marker”, or “biomarker” interchangeably refer to a molecule (typically nucleic acid, protein, proteolytic fragment, carbohydrate, or lipid) that is present in a biological sample, from a subject with cancer, expressed in a cancer cell, expressed on the surface of a cancer cell, or secreted by a cancer cell differentially in comparison to a biological sample from a subject without cancer or a non-cancer cell, and which is useful for the diagnosis of cancer, for providing a prognosis, or for preferential targeting of a pharmacological agent to the cancer cell. In the context of the present invention, a cancer-associated antigen may be a proteolytic fragment, for example one that is generated in response to an apoptotic stimulus, that is present in a biological sample, such as a blood sample, tumor biopsy, tissue, and the like, from a patient suffering from a disease, such as cancer, at an elevated level, for example, 10% greater level, 20%, 50%, 75%, 100% or greater level, than found in an biological sample from an individual not suffering from the disease. In other cases, the proteolytic fragment may be present at about 1-fold, 2, 3, 4, 5, 6, 7, 8, 9, 10, 20, 30, 40, 50, 75, 100, 250, 500, or 1000-fold greater level in a sample from a patient suffering from the disease as compared to a sample from an individual not suffering from the disease, or a control sample. In some embodiments, a biomarker of the present invention may be a proteolytic fragment that is present in a biological sample from a patient suffering from a disease, such as cancer, but not present, or present at a minimal level, in a sample from an individual not suffering from the disease. In other embodiments, a cancer-associated antigen is a molecule that is overexpressed in a biological sample from a subject with cancer or a cancer cell in comparison to a biological sample from a subject without cancer or a non-cancer cell, for instance, 1-fold over expression, 2-fold overexpression, 3-fold overexpression or more in comparison. Oftentimes, a cancer-associated antigen is a molecule that is inappropriately synthesized in a cancer cell or present in a biological sample from a subject with cancer, for instance, a molecule that contains deletions, additions or mutations in comparison to the molecule expressed in a biological sample from a subject without cancer or in a non-cancer cell.

The “proteolytic apoptotic polypeptide biomarkers” of the present invention generally relate to proteolytic polypeptides that are generated in response to an apoptotic stimulus. Typically, these fragments are formed by the cleavage of a “pro-apoptotic polypeptide” or “proteolytic apoptotic cleavage junction” by a protease involved in an apoptotic pathway. Typically, two proteolytic apoptotic polypeptide biomarkers are generated by every cleavage. For example, one proteolytic polypeptide may comprise an N-terminal sequence selected from those found in Table 4. I.e., cleavage of Serine/threonine-protein phosphatase 2A 56 kDa regulatory subunit gamma isoform, Swiss-Prot accession number Q13362, results in a proteolytic apoptotic polypeptide biomarkers comprising the sequence AANSNGPFQPVVLLHIR (SEQ ID NO:418), wherein AAN are the first three, or N-terminal, residues of the biomarker. A second proteolytic apoptotic polypeptide biomarker formed by a cleavage reaction may comprise a C-terminal sequence also found in Table 4. I.e., cleavage of Serine/threonine-protein phosphatase 2A 56 kDa regulatory subunit gamma isoform, Swiss-Prot accession number Q13362, will also result in a proteolytic apoptotic polypeptide biomarkers comprising the sequence AGSRMVVD (SEQ ID NO:419), wherein VVD are the last three, or C-terminal, residues of the biomarkers. In certain embodiments, proteolytic apoptotic polypeptide biomarkers of the invention may further comprise a fusion sequence N-terminal or C-terminal to a sequence found in Table 4, in order to facilitate purification or detection of the biomarker. Proteolytic apoptotic polypeptide biomarkers of the present invention may comprise polypeptides spanning from the cleavage site (P1 or P1′ residue) to the N- or C-terminus of the parent protein. In other embodiments, the proteolytic apoptotic polypeptide biomarkers of the invention may undergo further proteolysis prior to detection or quantitation. As such, a proteolytic apoptotic polypeptide biomarker may comprise at least about 4, 5, 6, 7, 8, 9, 10, 11, 12, 13, 14, 15, 16, 17, 18, 19, 20, 25, 30, 35, 40, 45, 50, 55, 60, 70, 80, 90, 100, 125, 150, 175, 200, 250, 300, 400, 500, or more of the parent protein, including the identified N- or C-terminal sequence, for example those found in Table 4.

A “proteolytic apoptotic cleavage junction”, or “cleavage junction”, or “intact cleavage junction”, in the context of the present invention, refers to an amino acid sequence, or polypeptide containing said sequence, that contains a recognition motif that is cleaved by a protease under certain conditions. In one embodiment, a cleavage junction of the invention is cleaved in response to an apoptotic stimulus. In a particular embodiment, the cleavage junctions comprise a sequence selected from those found in Table 4. A cleavage junction is said to correspond to a proteolytic polypeptide or a proteolytic apoptotic polypeptide biomarker if said proteolytic polypeptide is formed or generated by the proteolysis of the cleavage junction. Thus, typically a cleavage junction of the present invention will result in the formation of two proteolytic apoptotic polypeptide biomarkers that correspond to said intact cleavage junction. In one embodiment, a cleavage junction comprising a sequence selected from those found in Table 4, with a given Swiss-Prot accession number, will correspond to two proteolytic polypeptides, one comprising an N-terminal sequence selected from those found in Table 4 and one comprising a C-terminal sequence selected from those found in Table 4, with the same Swiss-Prot accession number. For example, a cleavage junction of Table 4, Swiss-Prot accession number Q13362, would correspond to both a proteolytic polypeptide comprising a N-terminal sequence of the corresponding unmodified polypeptide sequence and a proteolytic polypeptide comprising an C-terminal sequence of the corresponding previous amino acid sequence.

In certain embodiments, the cleaved products of the present invention may be further trimmed in vivo or in vitro by exoproteases after capsase-based proteolysis. The present invention, in one embodiment, includes fragments of the biomarkers identified herein that have been further processed by such exoproteases, which may serve as biomarkers of apoptosis equivalent to their predecessor fragments. In other embodiments, the detection of either an N-terminal or C-terminal proteolytic fragment, in the absence of the other, will provide diagnostic or prognostic power for the detection of spoptosis in a biological sample.

An “apoptotic stimulus” generally refers to a signal or condition that causes or induces a cell to undergo apoptosis. Apoptotic signals may originate intracellularly, as per the action of an intrinsic inducer, or extracellularly, as in the action of an extrinsic inducer. Extracellular signals may include, without limitation, toxins, hormones, growth factors, nitric oxide, cytokines, cytotoxic drugs, and the like. Intracellular apoptotic signalling is typically initiated in response to stress. These stimuli include, without limitation, the binding of nuclear receptors by glucocorticoids, heat, radiation, nutrient deprivation, viral infection, hypoxia, and the like. In certain embodiments of the invention, apoptosis may be induced through the use of cytotoxic drugs or by environmental conditioning of the cells.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum or saliva, lymph and tongue tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., tongue, colon, prostate, kidney, bladder, lymph node, liver, bone marrow, blood cell, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Nati. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of cancer antigens. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of a target protein or polypeptide.

The phrase “specifically (or selectively) binds” to or “specifically (or selectively) immunoreactive with” an antibody or binding reagent, when referring to a protein or peptide, refers to a binding reaction that is determinative of the presence of the protein, often in a heterogeneous population of proteins and other biologics. Similarly, an antibody or binding reagent is considered to “substantially bind” to an epitope, when the antibody or binding reagent binds to said epitope in a specific or selective fashion. Thus, under designated immunoassay conditions, the specified antibodies or binding reagents bind to a particular protein at least two times the background and more typically more than 10 to 100 times background. Specific binding to an antibody or binding reagent under such conditions requires an antibody or binding reagent that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies or binding reagents specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

A binding reagent or antibody that “binds with a lower affinity” to a second polypeptide or to a background polypeptide will generally bind specifically to a first target polypeptide of interest with a greater affinity as compared to the binding affinity to said second polypeptide. In certain embodiments, the binding reagent or antibody will bind to the second polypeptide with at least a two fold lower affinity, or more typically at least about 10-fold, 100-fold, or 1000-fold lower affinity as compared to the binding affinity of the first or target polypeptide. In this fashion, a binding reagent or polypeptide that binds with a lower affinity to a second polypeptide can discriminate between a first target polypeptide and a second polypeptide, even when the second polypeptide is a derivative of the first polypeptide. For example, an antibody specific for a proteolytic polypeptide of the present invention may bind with a lower affinity to the corresponding proteolytic cleavage junction, or a polypeptide containing said cleavage junction, such that the target proteolytic polypeptide, or the level thereof, can be discriminated from said cleavage junction in a biological sample.

In the context of the present invention, a disease is “characterized by apoptosis” if said disease results in altered levels of apoptosis in an individual suffering from the disease. A disease may be considered to be characterized by apoptosis, for example, if levels of apoptosis are reduced or increased in an individual suffering from the disease as compared to levels in individuals not suffering from said disease. In one embodiment of the present invention, apoptosis levels may be reduced or increased by at least about 5%, or at least about 10, 15, 20, 25, 30, 35, 40, 45, 50, 60, 70, 80, 90, or 100% as compared to levels in an individual not suffering from said disease. In other embodiments, the level of apoptosis may be reduced or increased by at least about 1-fold, 2-fold, 3-fold, 4-fold, 5-fold, 6-fold, 7-fold, 8-fold, 9-fold, 10-fold or more as compared to levels in individuals not suffering from the disease. In yet other embodiments, apoptosis may be reduced or increased by at least about 1 order of magnitude, or at least about 2, 3, 4, 5, 6, 7, 8, 9, 10, or more orders of magnitude as compared to levels in an individual not suffering from the disease. Non-limiting examples of diseases that are characterized by apoptosis include, cancer, auto-imune diseases (such as Graves' disease, Lupus erythematosus, Rheumatoid arthritis, Sjögren's syndrome, multiple sclerosis, type-I diabetes mellitus, Hashimoto thyroiditis, and the like), neurodegenerative diseases (such as Parkinson's or Alzheimer's Diseases), preeclampsia, acute and chronic liver diseases, and the like.

### Diagnostic Methods

The present invention provides methods of diagnosing a disease characterized by apoptosis, by examining proteolytic apoptotic biomarkers, including proteolytic polypeptides comprising an N-terminal or C-terminal sequence found in Table 4 in biological samples, including wild-type, truncated or alternatively spliced forms. Diagnosis involves determining the level of a polypeptide of the invention in a patient and then comparing the level to a baseline or range. Typically, the baseline value is representative of a polypeptide of the invention in a healthy person not suffering from the disease, as measured using biological sample such as blood, serum, saliva, urine, a tissue sample (e.g., tongue or lymph tissue), or a biopsy. Variation of the levels of a polypeptide of the invention from the baseline range (either up or down) indicates that the patient has a disease characterized by apoptosis or is at risk of developing a disease characterized by apoptosis.

Analysis of a protein can be achieved, for example, by high pressure liquid chromatography (HPLC), alone or in combination with mass spectrometry (e.g., MALDI/MS, MALDI-TOF/MS, tandem MS, etc.).

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used (see, e.g., Self et al., Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (META); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody or binding reagent to a protein can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the protein marker is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies or binding reagents can be immobilized onto a variety of solid supports, such as polystyrene beads, magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

Useful physical formats comprise surfaces having a plurality of discrete, addressable locations for the detection of a plurality of different biomarkers. Such formats include protein microarrays, or “protein chips” (see, e.g., Ng et al., J. Cell Mol. Med., 6:329-340 (2002)) and certain capillary devices (see, e.g., U.S. Pat. No. 6,019,944). In these embodiments, each discrete surface location may comprise antibodies to immobilize one or more protein markers for detection at each location. Surfaces may alternatively comprise one or more discrete particles (e.g., microparticles or nanoparticles) immobilized at discrete locations of a surface, where the microparticles comprise antibodies to immobilize one or more protein markers for detection.

The analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

### Compositions, Kits and Integrated Systems

The invention provides compositions, kits and integrated systems for practicing the assays described herein using polypeptides of the invention, antibodies or binding reagents specific for polypeptides of the invention, etc.

The invention provides assay compositions for use in solid phase assays; such compositions can include, for example, one or more antibodies or binding reagents specific for the polypeptide biomarkers of the invention immobilized on a solid support, and a labeling reagent. In each case, the assay compositions can also include additional reagents that are desirable for hybridization.

The invention also provides kits for carrying out the diagnostic or prognostic assays of the invention. The kits typically include a probe that comprises an antibody or binding reagent that specifically binds to polypeptides of the invention, and a label for detecting the presence of the probe. The kits may include several antibodies specific for the polypeptides of the invention. In one embodiment, the kits of the invention comprise at least 2, or at least about 3, 4, 5, 6, 7, 8, 9, 10, 15, 20, 25, 30, 40, 50, 60, 70, 80, 90, 100, 125, 150, 175, 200, 250, 300 or more antibodies or binding reagents.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical images.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen are sampled to obtain light intensity readings for each position. Multiple pixels are processed in parallel to increase speed. The apparatus and methods of the invention are easily used for viewing any sample, e.g., by fluorescent or dark field microscopic techniques.

### N-Terminal Labeling of Polypeptides

In general, any method of making an extract from cells or tissues from a biological sample that preserves the ability to label the N-termini of polypeptides with the reagents described below may be used in the practice of this invention. Any of a number of such methods are known in the art and are described in standard sources (see, e.g., Scopes, Protein Purification: Principles and Practice (1982)). In general, cells are disrupted to release and solubilize intracellular contents, followed by centrifugation to remove insoluble material, such as cell membranes and organelles. For tissue culture cells, a lysis buffer which may contain a detergent (e.g., Triton X-100, NP-40, among others) may be used. For adherent tissue culture cells, cell disruption can be accomplished by the process of scraping cells in the presence of the lysis buffer from culture plates using, for example, a rubber policeman. Other mechanical means can also be used to effect cell disruption. For example, cells can be lysed using a Dounce homogenizer. As recognized by the skilled artisan, additional mechanical means may be needed to prepare cell extracts from tissues, such as homogenization in a blender or sonication. (See, generally, e.g., Scopes, Protein Purification: Principles and Practice (1982).)

The labeling of polypeptides can be accomplished using any method that labels the N-terminus (i.e., α-amino group) of a polypeptide present in a complex mixture.

In one embodiment of this invention, the labeling is accomplished using the enzyme subtiligase, which is derived from the enzyme subtilisin BPN′ by converting the catalytic residue, Ser-221, to a cysteine residue, and Pro-225 to an alanine residue. The resulting double mutant protein provides the enzymatic activity of ligation of esterified peptides site-specifically onto the N termini of proteins or peptides (see, e.g., Chang, T. K. et al., Proc. Nati. Acad. Sci. U.S.A., 91, 12544-12548 (1994)). Furthermore, additional forms of subtiligase that exhibit increased stability have been generated through the introduction of additional site directed mutations into the sequence (e.g., Met-50 to Phe, Asn-76 to Asp, Asn-109 to Ser, Lys-213 to Arg, and Asn-218 to Ser). Such mutant enzymes have also been termed stabiligases and may also may be used in the practice of the present invention (see, e.g., Chang, T. K. et al., Proc. Natl. Acad. Sci. U.S.A., 91, 12544-12548 (1994)).

All of the earlier work describing the use of subtiligase and its variants disclosed the ligation of peptides and proteins in non-complex samples composed of single purified polypeptides. In this earlier work, two examples of the application of subtiligase to the ligation of proteins that were recombinantly expressed on the surface of phage particles were shown. For example, the work of Chang et al. demonstrated the ligation of phage-displayed human growth hormone variants that were randomized at the first three residues (Chang, T. K. et al., Proc. Natl. Acad. Sci. U.S.A., 91, 12544-12548 (1994)). The work of Atwell et al. demonstrated the autoligation of phage-displayed subtiligase variants that contained an N-terminal extension and were randomized at up to five different residues outside of this N-terminal extension (Atwell S. et al., Proc. Natl. Acad. Sci. U.S.A., 96, 9497-9502 (1999)). In contrast, the present invention represents a major advance, as it applies subtiligase to the ligation of polypeptides in complex mixtures of endogenous proteins as found in a variety of biological samples, not merely to simple formulations of recombinant proteins, as shown by the earlier studies. The modest amount of sample complexity in the earlier reported phage display experiments arises from minor genetic manipulations of either the human growth hormone gene or the subtiligase gene. In contrast, the complexity found in the biological samples of the present invention arises from the fact that the component polypeptides of the complex mixtures of the invention are products of a plurality of endogenous genes, which are subject to transcriptional, translational, and post-translational modulation of expression.

Furthermore, the work of Chang et al. demonstrated that subtiligase is very dependent on the primary and secondary structure of polypeptide substrates. Although subtiligase was found to exhibit broad specificity for peptide substrates, some N-terminal residues in these substrates were found to be exceedingly more preferred than others. Structural occlusion of N-termini in a protein substrate was also found to drastically affect ligation efficiency. This earlier work indicated limitations to this approach for labeling a plurality of polypeptides in complex mixtures and provided no indication of applicability to more complex samples, as the only substrates used in addition to short peptides were recombinant human growth hormone and subtiligase. In fact, those of skill in the art recognized several potential pitfalls in the implementation of subtiligase as a tool for selective labeling of polypeptide α-amines in complex mixtures. First, it was believed that only the most abundant proteins in the sample would be labeled. Second, the previous data indicated the possibility that only the most efficient substrates, based on the identity of N-terminal residues, would be labeled. Third, there existed the possibility of poor labeling of mixtures due to structural occlusion of N-termini. Fourth, there was a strong possibility that complex samples would contain inhibitors of subtiligase. Fifth, there was a prevalent concern that the peptide glycolate ester reagents would not be stable in biological samples because of the action of endogenous esterases and proteases.

However, as demonstrated below, it has surprisingly been found herein that these many pitfalls could be circumvented; indeed, it has been demonstrated that subtiligase may be used to efficiently label the N-termini of a plurality of polypeptides in complex mixtures, such as cell extracts and serum. For example, it has been shown that addition of a cocktail of inhibitors sufficiently blocks endogenous proteases and esterases without inhibiting subtiligase, thus, allowing for sufficient substrate to be available for ligation. Another advantage imparted by the present invention is the nature of the labeled peptide ester reagents used herein. Versions of these reagents have been designed that are optimized for use in proteomic studies. Among other innovations, it has been found that incorporation of a cleavable linker into these reagents greatly facilitates purification of labeled polypeptides from complex mixtures and subsequent analysis by tandem mass spectrometry for identification of the corresponding proteins.

Additional variants of subtiligase enzymes that have enhanced activity have also been selected through the application of phage display methods (see, e.g., Atwell, S. et al., Proc. Natl. Acad. Sci. U.S.A., 96:9497-502 (1999)). Such variants may also be used in the practice of the present invention. Furthermore, other subtilisin-like enzymes and their variants may also be engineered to be used in the practice of this invention.

Subtiligase has been used to incorporate a variety of label moieties into proteins and polypeptides, including affinity handles (e.g., biotin), immunoprobes, isotopic labels, heavy-atom derivatives, PEG moieties, and other non-natural constituents (see, e.g., Chang, T. K. et al., Proc. Natl. Acad. Sci. U.S.A., 91, 12544-12548 (1994)). The skilled artisan will recognize that this is not an exhaustive list, as for instance, any detectable label that can be incorporated into a substrate (e.g., biotin labeled peptide esters) to be used to label a free N-terminus (e.g., α-amino group of a polypeptide generated through proteolysis) may be used. In particular, any of the labels disclosed above may be used in the practice of the present invention.

The reaction by which subtiligase may be used to label a free N-terminus of a polypeptide with a biotin labeled peptide ester as the substrate for the introduction of a biotin label onto a protein has been previously described (see US Pat. Pub. No. 2012-0028266 A1). In the first step of this reaction, a free sulhydryl group on subtiligase serves as a nucleophile to effect a nucleophilic attack on the carbonyl carbon atom of the ester moiety of the substrate peptide ester, resulting in the release of an alcohol leaving group. In a second step, the carbonyl carbon of the thioester linkage between the peptide substrate and the subtiligase enzyme is then subject to nucleophilic attack by the α-amino group of a protein or peptide. This reaction results in a covalent adduct comprising the biotin labeled peptide linked to the α-amino group on a protein or peptide via an amide bond. Accordingly, the biotin label then can serve as an affinity handle to allow the identification and isolation of polypeptides that have a free N-terminus or free α-amino group (e.g., protein fragments that have resulted from proteolysis, or native non-acetylated or otherwise N-terminally blocked proteins).

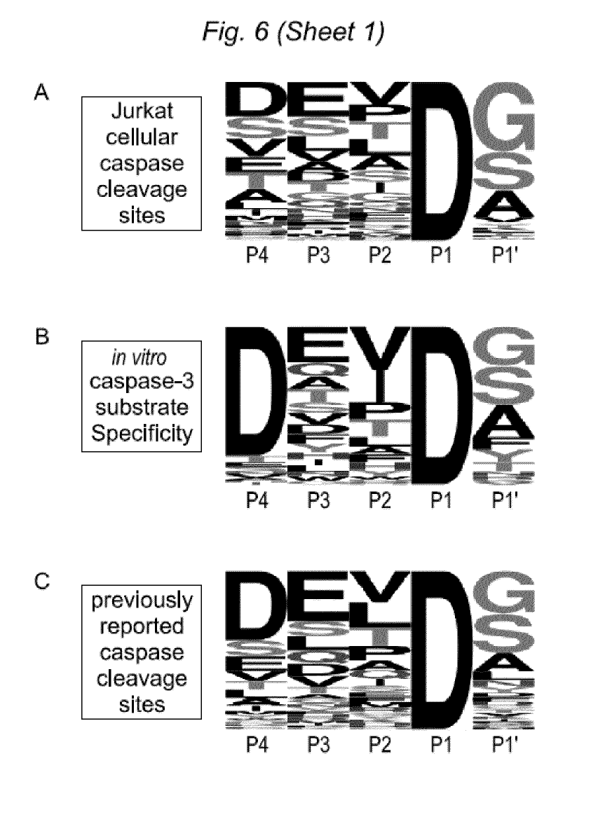

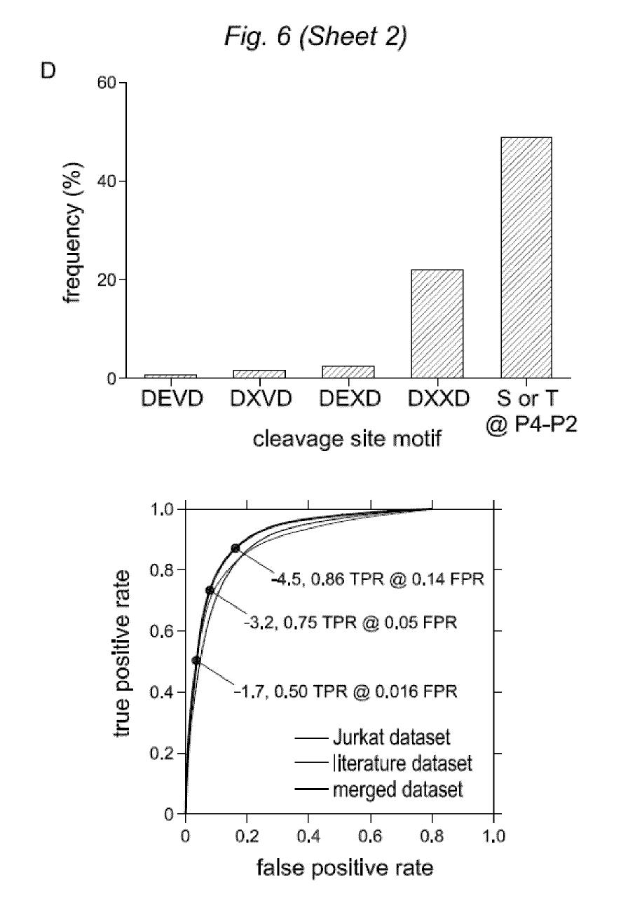

In general, any peptide ester with the following generic elements may be used in the practice of the present invention: label—linker—peptide sequence—esterified carboxyl terminus. The skilled artisan will recognize that the location of the label within this structure may be varied without affecting the operation of the present invention. The generic structure of these elements may optionally contain a protease cleavage site or other cleavable moiety to facilitate the ready removal of the label added to the α-amino group of a protein or polypeptide. Such removal also greatly facilitates downstream mass spectrometric analysis of labeled proteins or polypeptides. FIGS. 6A-6C show a representative peptide ester that may be used in the practice of the invention. In this example, there is a biotin label at the N-terminus of the peptide ester, a site for a protease cleavage (TEV protease), and an esterified carboxyl terminus, which serves as a subtiligase cleavage site (i.e., the site for the nucleophilic attack by a free sulfhydryl group on subtiligase as described above). Among the peptide sequences that may be used in the practice of the invention include, but are not limited to: ENLYFQSY (SEQ ID NO:420), ENLYFQSK (SEQ ID NO:421), ENLYFQSA (SEQ ID NO:422), AAPY (SEQ ID NO:423), AAPK (SEQ ID NO:424), and AAPA (SEQ ID NO:425), among others. Optional protease cleavage sites that may be used in the practice of this invention include, but are not limited to: the site for TEV protease: EXXYXQ(S/G/A) (SEQ ID NO:436), where X corresponds to any amino acid; the site for rhinovirus 3C protease: E(T/V)LFQGP (SEQ ID NO:426); the site for enterokinase: DDDDK (SEQ ID NO:427); the site for Factor Xa: I(D/E)GR; the site for thrombin: LVPR (SEQ ID NO:428); the site for furin: RXXR (SEQ ID NO:437), where X corresponds to any amino acid; and the site for Granzyme B: IEPD (SEQ ID NO:429). Some examples of the many possible moieties that may be used to esterify the carboxyl terminus of the peptide are: HO—CH2-CO—X, where X is any amino acid, in the case of glycolate esters; HO—CHCH3-CO—X, where X is any amino acid, in the case of lactate esters; HO—R, where R is an alkyl or aryl substituent; and HS—R, where R is an alkyl or aryl substituent. A number of label moieties may be used, including radioisotopes, stable isotopes, flurophores, heavy metals, and biotin, among others.

In general, any reaction conditions that favor nucleophilic attack of a carbonyl group at an ester or thioester linkage to result in the release of the relevant leaving group (e.g., an alcohol in step one or the —SH group of subtiligase in step two) may be used in the practice of this invention for the labeling of free α-amino groups. Generally, any conditions under which ester reagents are stable to degradation and hydrolysis in complex samples; conditions under which subtiligase is stable and active; and conditions under which protein and polypeptide N-termini are free and available to react with the thioester linkage formed after the reaction of subtiligase with ester reagents are favored for the practice of this invention.

In some embodiments of this invention, the pre-existing unblocked α-amino groups of polypeptides may be blocked with a suitable N-termini blocking agent before an experimental treatment. Thus, for instance, the free, unblocked N-termini of cellular proteins may be blocked with any reagent that reacts with free α-amino groups prior to exposure of a biological sample to an agent, such as a chemotherapeutic agent, which promotes a physiological response of interest, such as apoptosis. After the experimental treatment, the newly exposed N termini which have resulted from the proteolytic events that accompany apoptosis can then be labeled using subtiligase and the ester substrates of the present invention. Examples of such blocking agents include: amine-reactive reagents such as succinimidyl esters, isothiocyanates, sulfonyl chlorides, and aldehydes, among others, provided these reagents do not contain primary or secondary amine moieties. In one embodiment, the blocking reaction can be accomplished using subtiligase and an acetylated ester.

It will be appreciated by the skilled artisan that a variety of complex samples can be labeled using the methods and compositions of the present invention. Such samples may include, without limitation, whole cells, cell extracts, media from cell cultures, serum from humans or animals, and other bodily fluids, among others. For example, the culture medium of cells stimulated with an agent that causes polypeptide secretion can be labeled using the methods of the present invention to identify polypeptides that have been secreted. As another example, proteins found on the surfaces of intact cells may be labeled to identify cell surface proteins, such as membrane proteins. The comparison of the cell surface proteins labeled in normal versus transformed cells can be used to identify, for example, tumor specific antigens. As a further example, serum or other bodily fluids from normal subjects and patients suffering from various diseases can be labeled to identify proteins that are unique to the serum of a patient population. The proteins so identified can serve as easily detected disease markers to be used in disease diagnostics. U.S. patent application Ser. No. 12/524,557 filed on Jul. 24, 2009, assigned to the same assignee as the present invention, the disclosure of which is incorporated by reference in its entirety and with particularity with reference to its teachings concerning methods for the specific N-terminal labeling and detection of peptides and proteins in complex mixtures.

### EXAMPLES

### Example 1

This example demonstrates the identification and profiling of N-termini in normal Jurkat cells.

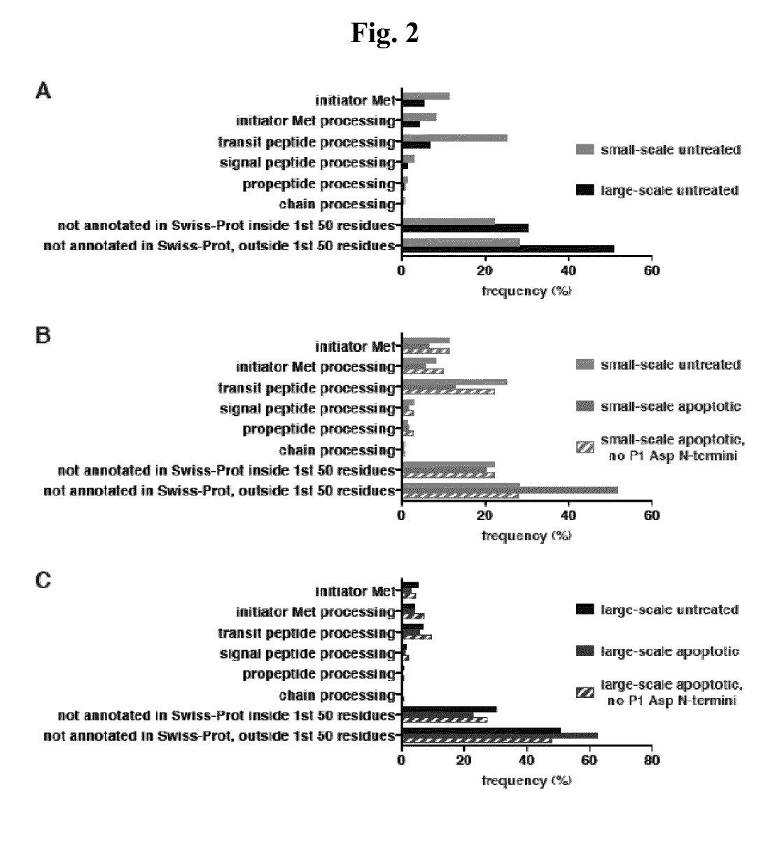

As a validation of a method provided by the present invention, endogenous N-termini in non-apoptotic Jurkat cells were analysed in two small-scale experiments using one-dimensional reversed-phase (1D) LC/MS/MS and two large-scale experiments using two-dimensional strong cation exchange/reversed-phase (2D) LC/MS/MS. Comparison of data obtained in both types of experiments is informative since 1D LC/MS/MS typically results in identification of abundant N-termini, whereas the increased proteomic coverage afforded by 2D LC/MS/MS results in additional identification of lower abundance N-termini. Of the combined 131 unique N-termini identified in small-scale experiments, 72% are either annotated in Swiss-Prot as native protein N-termini, or correspond to cleavages within the first 50 residues of proteins as would be expected for N-terminal signal or transit peptide processing (FIG. 2A). The remaining 28% correspond to cleavages outside the first 50 residues, arising from additional processing or constitutive protein degradation. In support of the latter notion, 51% of the combined 661 unique N-termini identified in large-scale experiments correspond to cleavages outside the first 50 residues (FIG. 2A). The increased frequency of such N-termini in large-scale experiments is consistent with the expected lower abundance for products of constitutive protein degradation.

### Example 2

This example provides degradomic analysis of apoptotic Jurkat cells.

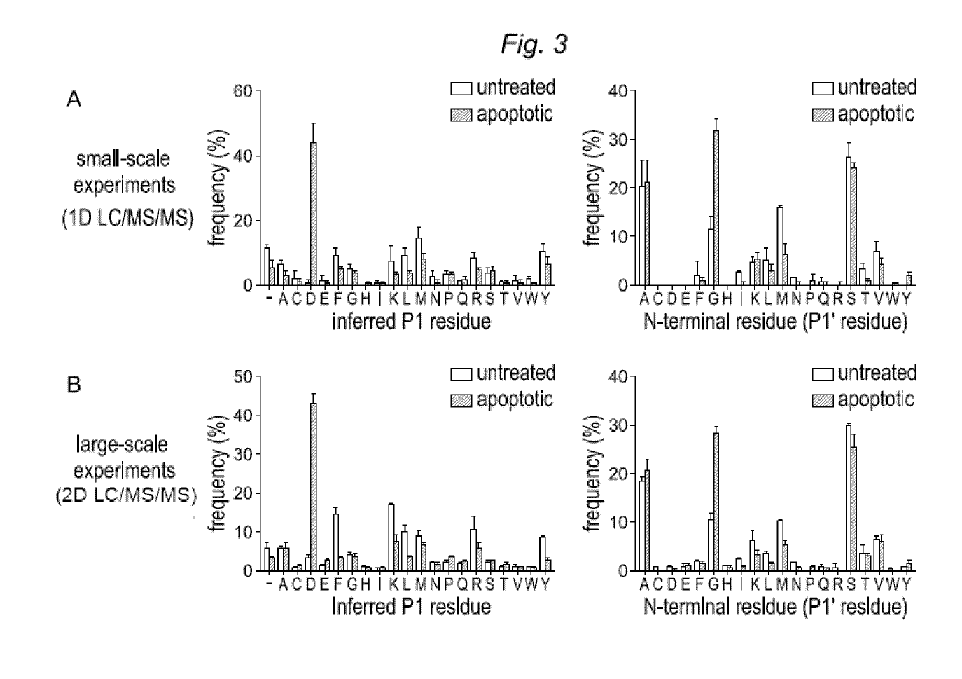

For analysis of apoptosis in Jurkat cells, several small-scale (1D) and large-scale (2D) LC/MS/MS experiments were carried out using cells treated with the topoisomerase II poison etoposide. The experiments with untreated cells described above serve as respective controls for the small- and large-scale experiments with apoptotic cells, in which a combined 244 and 733 unique N-termini, respectively, were identified. Caspases are known to exhibit strict substrate specificity for aspartate at P1, and for glycine>serine>alanine at P1′ (Schilling et al., Nat Biotechnol. 2008; 26(6):685-94; Stennicke et al., Biochem J. 2000; 350 Pt 2:563-8). In small-scale experiments, 43% of N-termini identified in apoptotic cells were derived from P1 aspartate cleavages, in contrast to less than 1% in untreated cells (FIG. 3A). In large-scale experiments, 43% of N-termini identified in apoptotic cells were derived from P1 aspartate cleavages, in contrast to 3% in untreated cells (FIG. 3B). An increased frequency of glycine at the first position of N-termini is also observed in apoptotic cells relative to untreated cells at both experimental scales (FIGS. 3A and 3B). The N-termini uniquely identified in apoptotic Jurkat cells are thus consistent with induction of caspase-like activity.

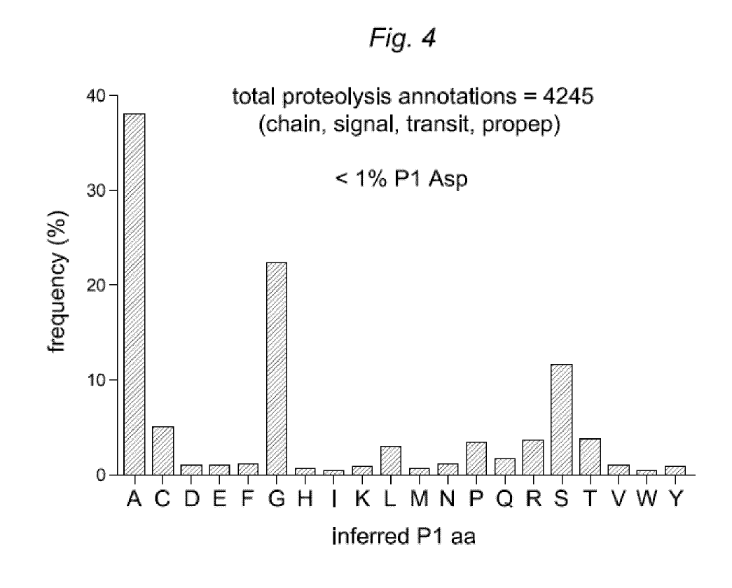

Of the 3% P1 aspartate N-termini detected in large-scale experiments with untreated cells (FIG. 3B), 55% correspond to reported caspase substrates (Liithi et al., Cell Death Differ. 2007; 14(4):641-50). Thus, it is likely that these originate from the small number of apoptotic cells typically present in untreated cultures. The detection of 3% P1 aspartate N-termini in large-scale experiments with untreated cells and less than 1% in small-scale experiments is consistent with the low abundance of such N-termini in cultures of normal cells. Additionally, if one considers that N-termini annotated in Swiss-Prot are representative of native N-termini in healthy cells, it is notable that <1% are derived from proteolytic processing following an aspartate residue (FIG. 4). In apoptotic samples, the increased frequency of N-termini located beyond the first 50 residues is solely attributable to P1 aspartate N-termini (FIGS. 2B and 2C). Thus, the vast majority of proteolysis we observe in apoptosis is attributable to caspases or proteases with caspase-like substrate specificity.

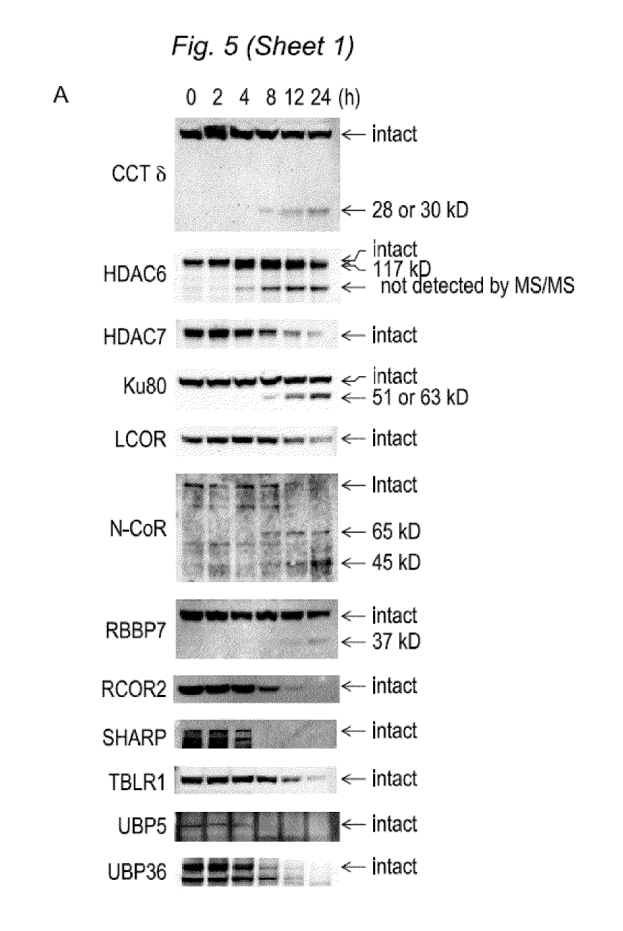

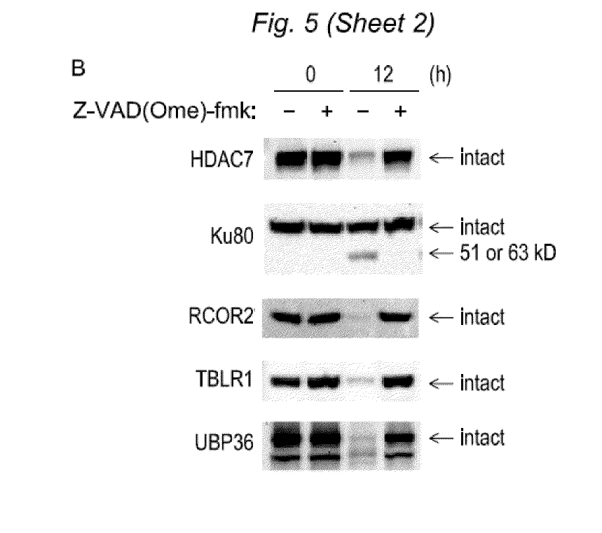

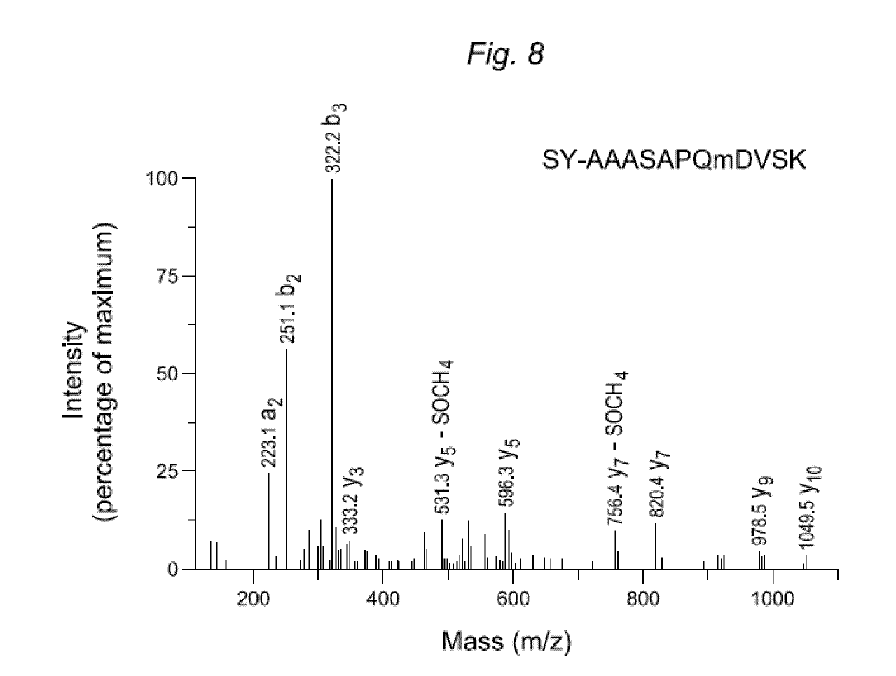

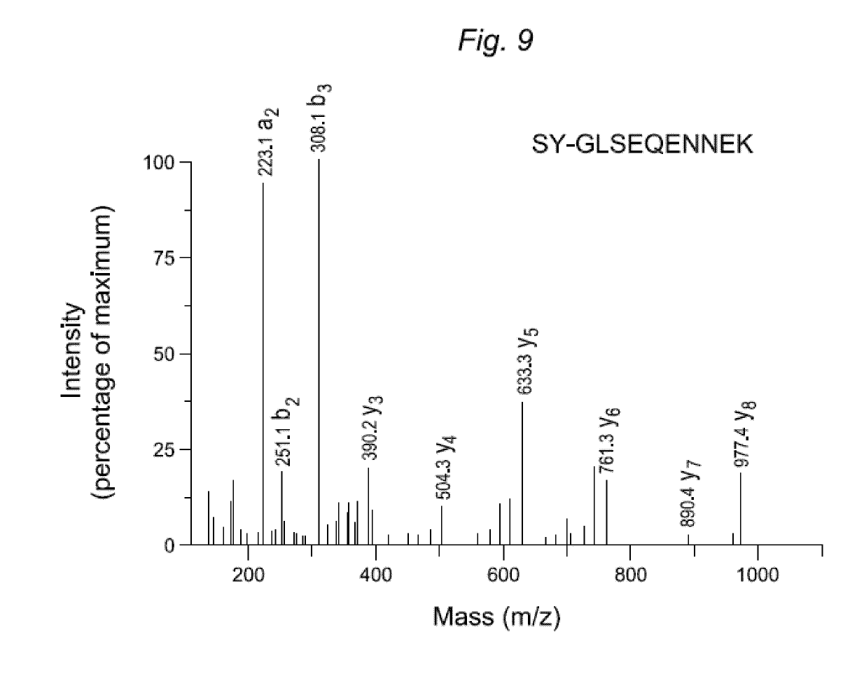

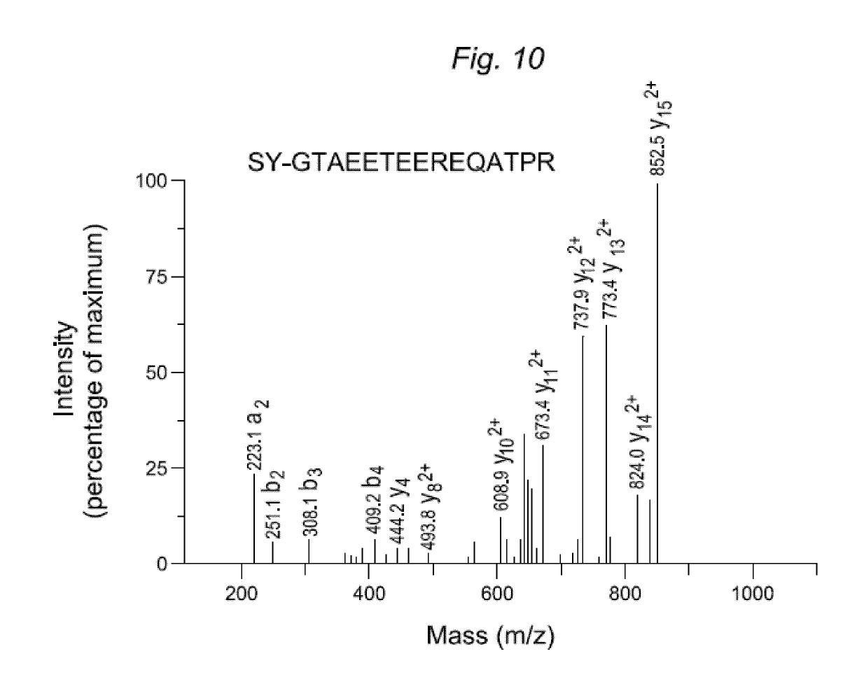

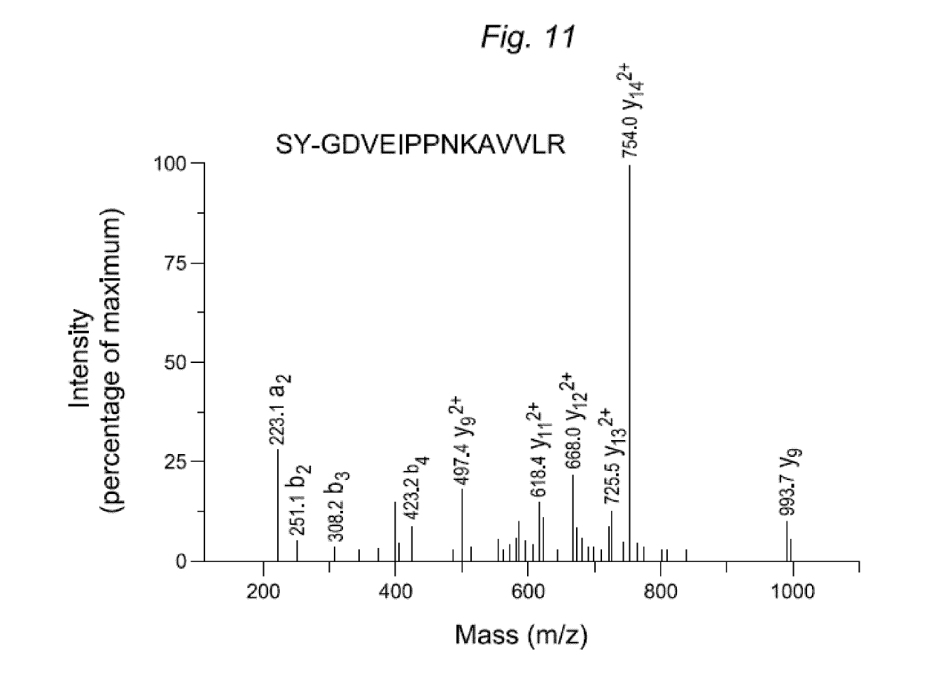

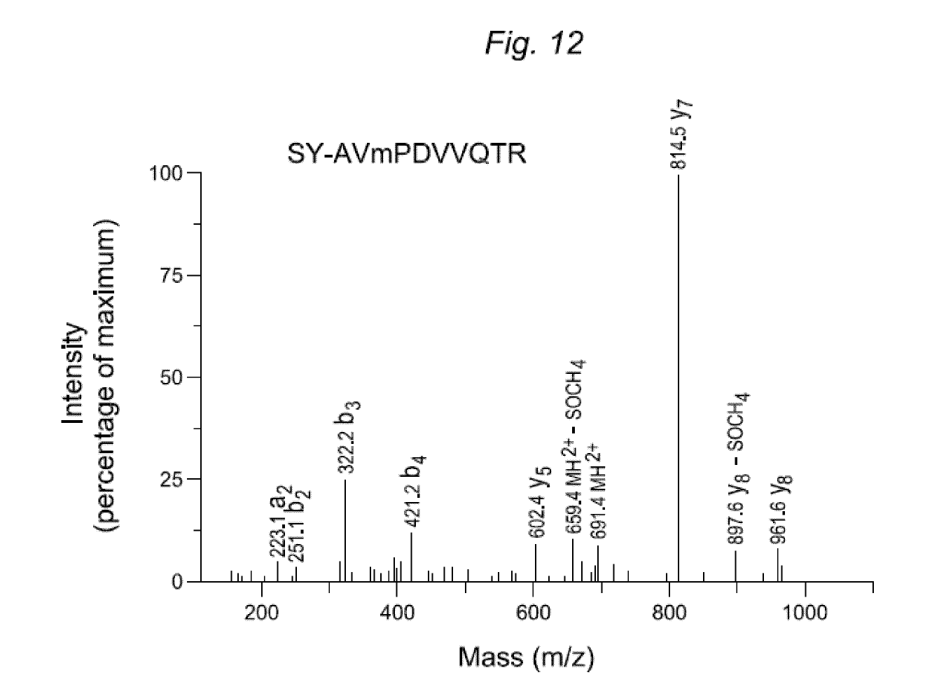

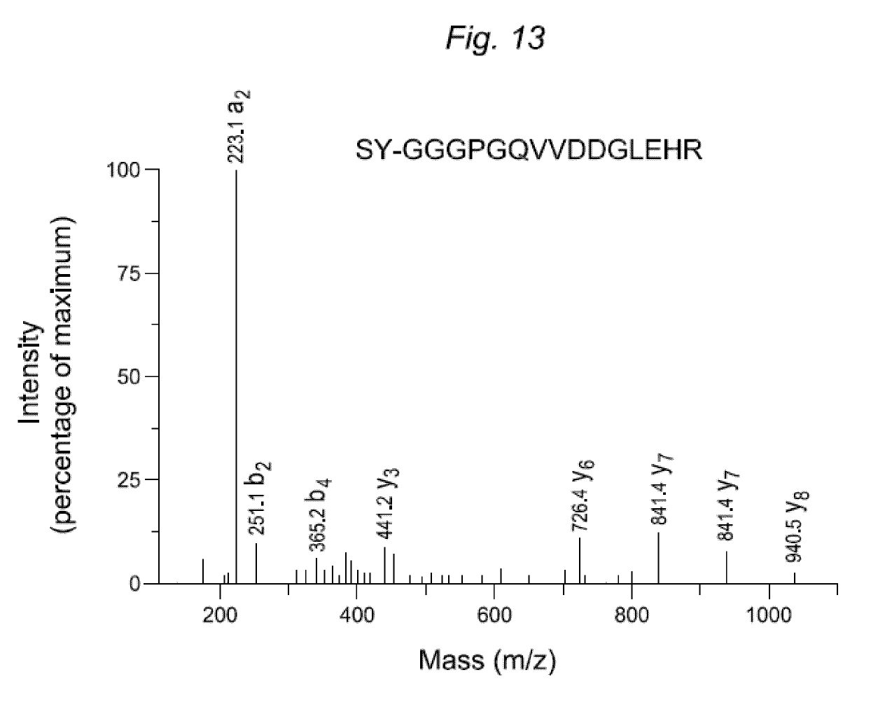

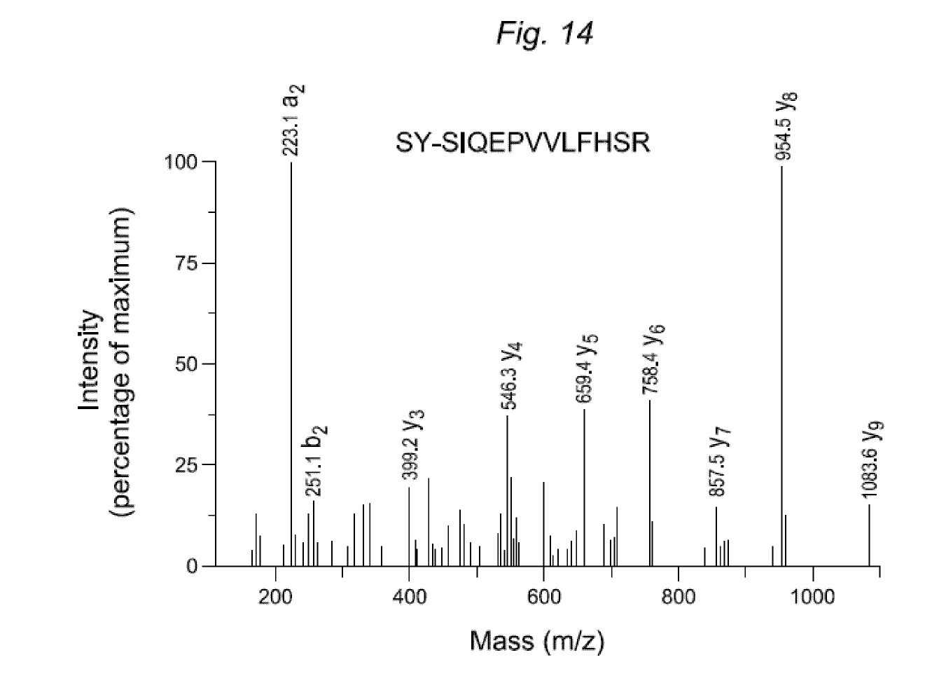

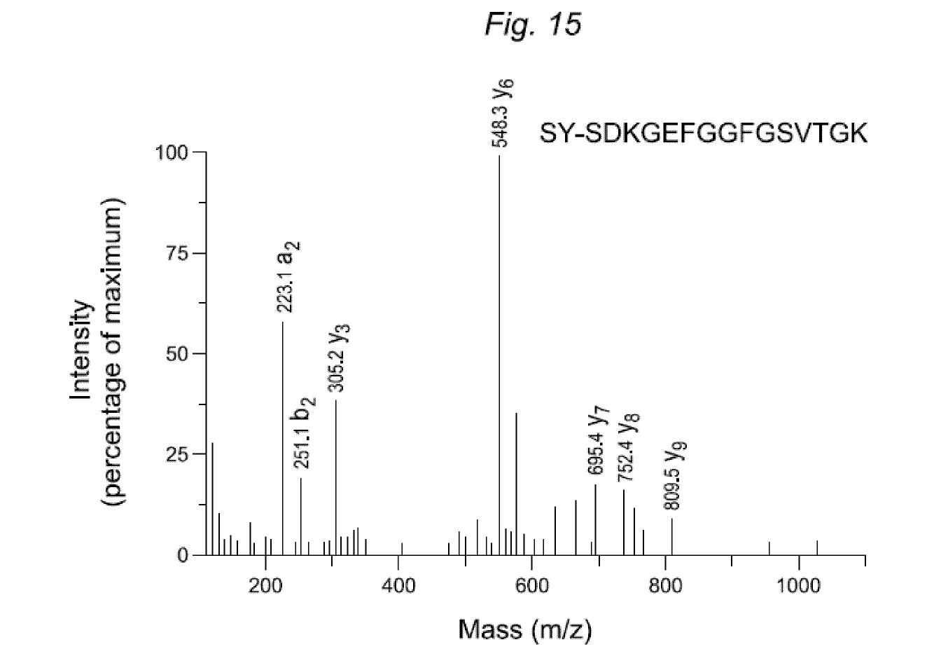

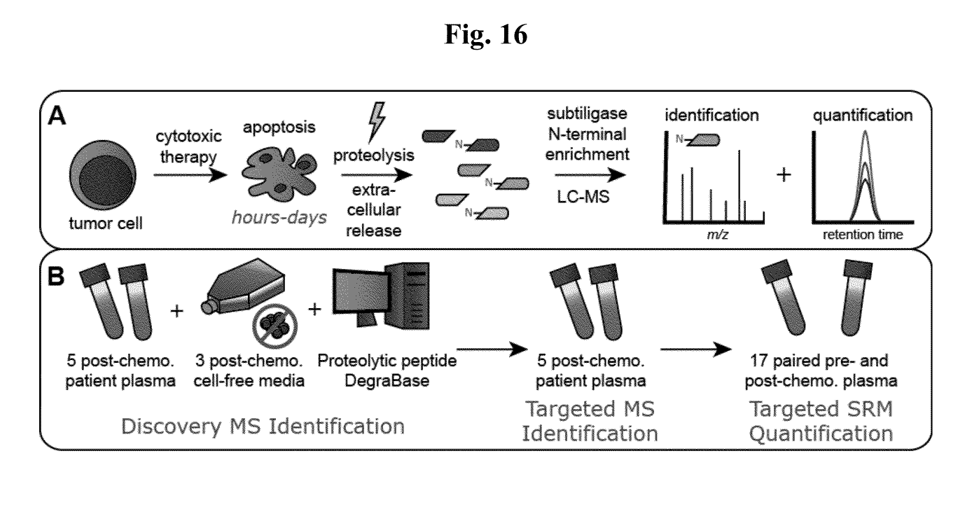

Among the total 1099 SY-labeled peptides identified in etoposide-treated Jurkat cells, 418 follow aspartate in corresponding protein sequences. These peptides correspond to 333 P1 aspartate N-termini and caspase-like cleavage sites. In turn, these cleavage sites map to 282 unique substrates and 10 additional others that cannot be distinguished from homologs containing the same identified cleavage site. Approximately 16 of the proteins identified as caspase substrates in these studies have been verified to be cleaved during apoptosis using immunoblotting (representative examples are indicated in FIG. 5A). The proteolysis of a representative set of substrates is also blocked by the broad-spectrum caspase inhibitor Z-VAD(OMe)-fmk, consistent with this proteolysis being caspase-dependent (FIG. 5B). Representative CID spectra for P1 aspartate peptides are included (FIGS. 8-15).

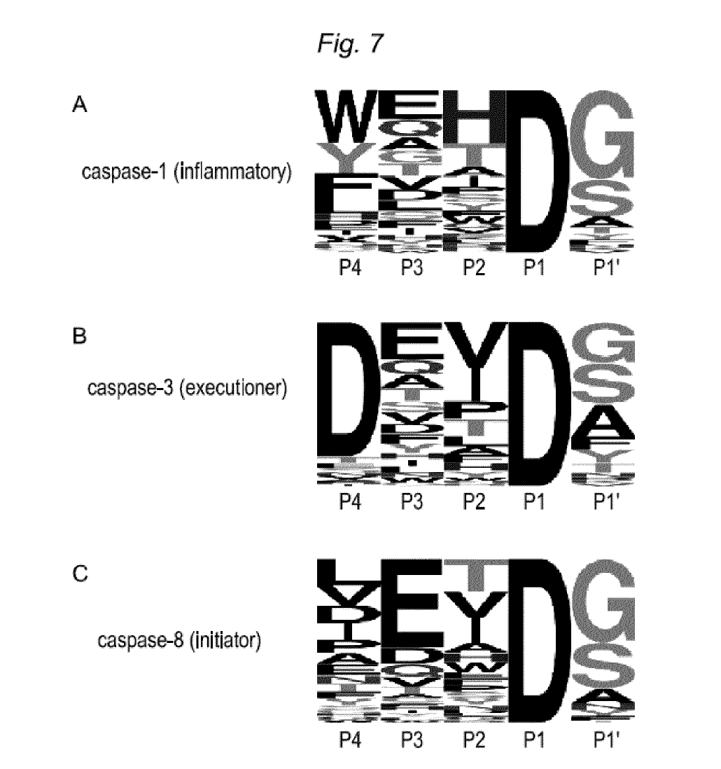

The most frequent residues at the P4, P3, P2, and P1′ positions of the caspase-like cleavage sites identified in apoptotic Jurkat cells are aspartate, glutamate, valine, and glycine, respectively (FIG. 6A). Thus, an averaged composite of these cleavage sites indicates that the most common caspase activity in apoptotic cells exhibits a specificity that is most similar to the substrate specificity of executioner caspases-3 and -7, as determined using peptide substrates (FIG. 6B) (Thornberry et al., J Biol Chem. 1997; 272(29):17907-11). However, there are significant differences between the cellular cleavage sites and the in vitro specificity profiles. Notably, the canonical DEVD (SEQ ID NO:430) cleavage site motif is found in less than 1% of the caspase-like cleavage sites observed in apoptotic Jurkat cells, and the broader DXXD (SEQ ID NO:438) motif is still only found in 22% of the identified cleavage sites (FIG. 6D). A distinct difference in the composite cellular profile is the high frequency of serine and threonine residues at P4, P3, and P2, which is not observed in vitro for any of the caspases (FIG. 7). Interestingly, a composite of all previously reported human and human ortholog of rodent caspase cleavage sites (Liithi et al., Cell Death Differ. 2007; 14(4):641-50.) is very similar to the Jurkat cellular profile reported here (FIG. 6C).

These observations suggest that caspase substrate specificity determined using peptide substrates has limited value as a predictor of physiological caspase cleavage sites. To investigate the predictive value of a large set of known physiological caspase cleavage sites, we constructed three profile hidden Markov models (HMMs) using the cleavage sites identified in our studies, previously reported cleavage sites, and the union of these two datasets. The accuracy of these HMMs was estimated using jacknifing and plotted in a receiver operator characteristic (ROC) plot, showing the true positive rate versus the false positive rate at different HMM score thresholds. While all three HMMs predict caspase cleavage sites relatively accurately, the HMM built from the merged substrate set performed most accurately (FIG. 6E).

### Example 3

### Cell Culture, Induction of Apoptosis, and Cell Lysate Preparation

Jurkat clone E6-1 (ATCC) cells were grown in RPMI-1640 supplemented with 10% fetal bovine serum, sodium pyruvate, and antibiotics. Normal cells were harvested for experiments at a density of 1×10⁶ cells/ml. For apoptotic samples, cells at a density of 1×10⁶ cells/ml were treated with etoposide (50 μM) for 12 hours prior to harvesting. Harvested cells (0.1 to 1 billion) were pelleted at 2,000×g and 25° C. for 5 minutes, washed twice with phosphate buffered saline, and lysed at a typical concentration of 2×10⁸ cells/ml in 1.0% Triton X-100, 100 mM BICINE pH 8.0, 100 μM Z-VAD-FMK, 100 μM E-64, 1 mM PMSF, 1 mM AEBSF, and 5 mM EDTA. Cell lysates were incubated at room temperature for 1 hour to allow complete inhibition of endogenous protease and esterase activity, and centrifuged at 21,000×g and 4° C. for 15 minutes to pellet insoluble material. Clarified supernatant was immediately used in ligation reactions at a protein concentration of approximately 20 mg/ml, as determined by Bradford assay (Bio-Rad).

### Example 4

### Sample Biotinylation, Denaturation, Reduction, Alkylation, and Gel Filtration

Subtiligase (1 μM), biotinylated peptide ester (1 mM), and DTT (2 mM) were added to either control or apoptotic cell lysate. Ligation reactions were typically left to proceed at room temperature for 60 minutes. Samples were then denatured by direct addition of solid guanidine hydrochloride to a final concentration of 6 M, reduced by addition of neutralized TCEP (2 mM), heated at 95° C. for 15 minutes, cooled to room temperature, and alkylated by addition of iodoacetamide (6 mM) and incubation at room temperature in the dark for 1 hour. The alkylation reaction was quenched by addition of DTT (10 mM), the sample was passed through a 0.8 μm filter, and subjected to gel filtration chromatography using a Superdex 30 16/60 column (GE Healthcare) on an AKTA FPLC system (GE Healthcare). The mobile phase was 100 mM BICINE pH 8.0, 200 mM NaCl, and 1 M guanidine hydrochloride. Fractions containing protein (corresponding to polypeptides greater than 5 kDa) were collected and pooled for a final volume of approximately 30 ml.

### Example 5

### Trypsinization and Recovery of Biotinylated Peptides

The gel-filtered material was supplemented with CaCl₂ (20 mM) and digested with sequencing grade modified trypsin (100 Promega) by incubation at 37° C. for 12 hours. Trypsinized samples were clarified by centrifugation, supplemented with benzamidine (500 mM), and NeutrAvidin agarose (250 μl bed volume, Pierce) was added for affinity capture of biotinylated N-terminal peptides. After 12 hours of gentle agitation, NeutrAvidin agarose resin was pelleted and washed with 100 mM BICINE pH 8.0 and AEBSF (1 mM), 100 mM BICINE pH 8.0, 5 M NaCl, and again with a few washes of 100 mM BICINE pH 8.0. More stringent washes using either 1 M or 5 M guanidine hydrochloride were used in some cases. Captured peptides were released from NeutrAvidin agarose resin by treatment with TEV protease (1 μM) in 100 mM BICINE pH 8.0 and DTT (1 mM). Recovered peptides were concentrated and desalted using ZipTip_{CIS} pipette tips, or a C₁₈ Macrotrap (Michrom) trap column on a 2796 HPLC system (Waters). TEV protease was sometimes depleted from samples using an SCX Macrotrap (Michrom) trap column.

### Example 6

### Expression and Purification of Subtiligase

The expression construct for subtiligase was prepared using the B. subtilis/E. coli shuttle vector pBS42 (ATCC) (Wells et al., Nucleic Acids Res. 1983; 11(22):7911-25). The variant of subtiligase used corresponds to subtilisin BPN′ containing point mutations S221C, P225A, M124L, and S125A for ligase activity (Abrahmsen et al., Biochemistry. 1991; 30(17):4151-9; Atwell et al., Proc Natl Acad Sci USA. 1999; 96(17):9497-502), and point mutations M50F, N76D, N109S, K213R, AND N218S for increased stability (Chang et al., Proc Natl Acad Sci USA. 1994; 91(26):12544-8). Recombinant subtiligase was prepared in B. subtilis strain 168 (ATCC). Subtiligase expression and purification was carried out essentially as described (Abrahmsen et al., Biochemistry. 1991; 30(17):4151-9). The purified enzyme was stored at −80° C. in 100 mM BICINE, pH 8.0 and 10 mM DTT or TCEP.

### Example 7

### Synthesis of Peptide Ester Substrates

Peptide glycolate ester substrates for subtiligase were prepared by solid-phase peptide synthesis using Fmoc chemistry as previously described (Braisted et al., Methods Enzymol. 1997; 289:298-313). Peptides were purified using 10×50 mm XTerra Prep MS C₁₈ ODB columns on a Parallex Flex HPLC system (Biotage). Purity and identity of peptides was verified by LC/MS analysis using a 4.6×50 mm XTerra MS C₁₈ column on a 2795 HPLC (Waters) system equipped with a ZQ quadrupole MS detector (Waters).

### Example 8

### Sample Fractionation Using Strong Cation Exchange (SCX) Chromatography

For larger scale experiments, samples were fractionated by SCX chromatography prior to LC/MS/MS analysis using a 2.1×200 mm PolySULFOETHYL Aspartamide column (The Nest Group) at a flow rate of 0.3 ml/min on a 2796 HPLC system (Waters). Buffer A consisted of 25 mM ammonium formate pH 2.8 and 30% acetonitrile, and buffer B consisted of 500 mM ammonium formate pH 2.8 and 30% acetonitrile. Approximately 25 fractions were collected during a 40 minute gradient block from 0% to 75% buffer B. Solvent from fractions was removed using an EZ-2 Plus evaporator (GeneVac), and remaining ammonium formate salt was removed by lyophilization. Some samples were also fractionated using a phosphate buffer and KCl salt system, in which case each fraction was subjected to automated desalting using a C₁₈ Microtrap (Michrom) trap column on a 2796 HPLC system (Waters) before solvent removal.

### Example 9

### Nano-LC-ESI-Qq-TOF MS/MS Analysis

Desalted fractionated or unfractionated samples were separated using a 75 μm x 15 cm C₁₈ column (LC Packings) at a flow rate of 350 nl/min, with a 60 minute gradient of 3 to 30% acetonitrile in 0.1% formic acid, on a 1100 series HPLC system (Agilent). The LC eluent was coupled to a microion spray source attached to a QSTAR Pulsar or QSTAR XL mass spectrometer (Applied Biosystems). Peptides were analyzed in positive ion mode. MS spectra were acquired for 1 s. For each MS spectrum, either the single most intense or the two most intense multiply charged peaks were selected for generation of subsequent CID mass spectra, depending on the analysis method used. The CID collision energy was automatically adjusted based upon peptide charge and m/z ratio. A dynamic exclusion window was applied that prevented the same m/z from being selected for 3 min after its initial acquisition. Representative CID spectra are included as FIGS. 8-15.

### Example 10

### Interpretation of MS/MS Spectra

Data were analyzed using Analyst QS software (version 1.1), and MS/MS centroid peak lists were generated using the Mascot.dll script (version 1.6b16). Data were searched against the Swiss-Prot human database (March 2008 release) using Protein Prospector 5.0 (University of California, San Francisco). Initial peptide tolerances in MS and MS/MS modes were 200 ppm and 300 ppm, respectively. The digest protease specified was trypsin, allowing for non-specific cleavage at N-termini in searches for N-terminally labeled semitryptic peptides, and trypsin allowing for non-specific cleavage at 0 N-termini in searches for unlabeled fully tryptic peptide contaminants. Two missed cleavages were typically allowed in searches. An N-terminal SY modification was specified as a fixed modification in searches for Nterminal peptides, but not in searches for unlabeled peptides. Cysteine carbamidomethylation was specified as a fixed modification and methionine oxidation was specified as a variable modification in all searches. High scoring peptide identifications from individual LC/MS/MS runs were then used to internally recalibrate MS parent ion m/z values within each run. Recalibrated data files were then searched again with an MS peptide tolerance of 100 ppm. Peptides with scores of greater than or equal to 22 and expectation values of less than or equal to 0.05 were considered positively identified. False positive rates for peptide identifications were estimated by conducting searches using a concatenated database containing the original Swiss-Prot human database, as well as a version of each original database entry where the sequence had been randomized. The overall false positive rate for N-terminal peptides identified was found to be 2.09%, while the false positive rate for peptides following aspartic acid in corresponding protein sequences was found to be 0.71%. A representative sampling of SY-labeled peptide identifications, particularly those based on expectation values near 0.05, was also manually validated.

### Example 11

### Immunoblotting and DNA Fragmentation Analysis

Jurkat cells at a density of 1×10⁶ cells/ml were treated with etoposide (50 μM) for 0, 2, 4, 8, 12, and 24 hours prior to harvesting. Harvested cells were pelleted at 2,000×g and 25° C. for 5 minutes, washed twice with phosphate buffered saline, and lysed at a concentration of 2×10₇ cells/ml in 1.0% SDS, phosphate buffered saline, 100 μM Z-VAD-FMK, 100 μM E-64, 1 mM PMSF, 1 mM AEBSF, 5 mM EDTA, and 10 mM sodium butyrate. Whole cell lysates were sonicated to shear genomic DNA, normalized to a protein concentration of approximately 2 mg/ml, as determined by BCA assay (Pierce). Cell lysates for each apoptotic timepoint were then analyzed by SDS-PAGE and Western blot. Mouse monoclonal anti-caspase-3 (#9668) and rabbit polyclonal anti-HDAC3 (#2632) antibodies were purchased from Cell Signaling Technology. Mouse monoclonal anti-DFF45 (#611036) antibody was purchased from BD Transduction Laboratories. Goat polyclonal anti-N-Cor (#sc-1611) and rabbit polyclonal anti-HDAC7 (#sc-11412) antibodies were purchased from Santa Cruz Biotechnology. Rabbit polyclonal anti-TBLR1 (#A300-408A), rabbit polyclonal anti-SHARP (#A301-119A), and rabbit polyclonal anti-RBBP7 (#A300-959A) antibodies were purchased from Bethyl Laboratories. Rabbit polyclonal anti-SMRTe (#06-891) antibody was purchased from Millipore. Western blots were imaged using SuperSignal West Femto Substrate (Pierce) with a FluorChem SP imager (Alpha Innotech). DNA fragmentation of whole cell DNA was analyzed by agarose gel with the Apoptotic DNA Ladder Kit (Roche).

### Example 12

Identification of protein N-termini in serum. Serum and plasma can be labeled by N-terminal protein biotinylation by a process similar to that described in Example 6. For example, two milliliters of human serum (NHS) supplemented with 100 mM BICINE pH 8.0, 1 mM EDTA, 1 mM PMSF, and 10% DMSO are labeled with 1 mM of biotinylated peptide ester using 1 μM subtiligase at room temperature for 15 to 120 minutes. Peptides corresponding to protein N termini of serum or plasma proteins are then recovered and identified as described in the Examples above. As a result of such an analysis, 79 nonredundant peptides can be identified in a single LC/MS/MS run, corresponding to 34 unique proteins. 68% of the peptides corresponded to annotated N termi resulting from signal cleavage or other known functional proteolytic processing. The 32% of N-terminal peptides with unknown origin indicate the potential of this technique to identify previously unknown posttranslational modifications in serum proteins. The abundances of identified proteins can span five orders of magnitude, from the processed N terminus of serum albumin (˜20 mg/ml) to insulin-like growth factor II (˜500 ng/ml). Low abundance serum proteins can be identified despite no effort being made to deplete highabundance proteins prior to analysis, illustrating the power of this labeling technique to partially neutralize dynamic range problems that confound serum proteomics. These results can be obtained without pre-fractionation of the labeled serum peptides. Significantly improved depth of coverage can be obtained with SCX fractionation.

Table 1 presents previously identified caspase-derived peptides. Previous residues indicates the inferred P8-P1 residues in the given protein substrate that directly precede the sequence of residues corresponding to the identified peptide. “Unmodified peptide” indicates the sequence of residues corresponding to the identified peptide. “Modified peptide” indicates the peptide as identified, sometimes containing chemical modifications such as oxidized methionine and carbamidomethylated cysteine, and always containing either an N-terminal serinyl-glycyl dipeptide (SerTyr) modification or an N-terminal 2-aminobutyryl (Abu) modification. Start residue (SR) indicates the residue number in the full-length protein sequence of the first residue of the unmodified peptide. “M” indicates the number of matches.

TABLE 1

| SEQ | | | | | | |
|---|---|---|---|---|---|---|
| ID | Swiss-Prot | Swiss- | | | | |
| NO. | ID | Prot acc # | unmodified peptide | SR | M | protein name |
| | 2A5G_HUMAN | Q13362 | | 15 | 1 | Serine/threonine-protein |
| | | | | | | phosphatase 2A 56 kDa |
| | | | | | | regulatory subunit gamma |
| | | | | | | isoform |
| 1 | 3MG_HUMAN | P29372 | AAQAPAEQPHSSS | 37 | 1 | DNA-3-methyladenine |
| | | | DAAQAPCPR | | | glycosylase |
| | 41_HUMAN | P11171 | | 551 | 1 | Protein 4.1 |
| | 41_HUMAN | P11171 | | 551 | 1 | Protein 4.1 |
| | 4EBP1_HUMAN | Q13541 | | 26 | 1 | Eukaryotic translation |
| | | | | | | initiation factor 4E-binding |
| | | | | | | protein 1 |
| | 4EBP2_HUMAN | Q13542 | | 27 | 1 | Eukaryotic translation |
| | | | | | | initiation factor 4E-binding |
| | | | | | | protein 2 |
| | A26CA_HUMAN | Q6S8J3 | | 945 | 10 | ANKRD26-like family C |
| | | | | | | member 1A |
| | A26CB_HUMAN | A5A3E0 | | 945 | | ANKRD26-like family C |
| | | | | | | member 1B |
| | ACTA_HUMAN | P62736 | | 247 | | Actin, aortic smooth muscle |
| | ACTBL_HUMAN | Q562R1 | | 246 | | Beta-actin-like protein 2 |
| | ACTB_HUMAN | P60709 | | 245 | | Actin, cytoplasmic 1 |
| | ACTC_HUMAN | P68032 | | 247 | | Actin, alpha cardiac muscle 1 |
| | ACTG_HUMAN | P63261 | | 245 | | Actin, cytoplasmic 2 |
| | ACTH_HUMAN | P63267 | | 246 | | Actin, gamma-enteric |
| | | | | | | smooth muscle |
| | ACTK_HUMAN | Q9BYX7 | | 245 | | Kappa-actin |
| | ACTS_HUMAN | P68133 | | 247 | | Actin, alpha skeletal muscle |
| | A26CA_HUMAN | Q6S8J3 | | 945 | 10 | ANKRD26-like family C |
| | | | | | | member 1A |
| | A26CB_HUMAN | A5A3E0 | | 945 | | ANKRD26-like family C |
| | | | | | | member 1B |
| | ACTA_HUMAN | P62736 | | 247 | | Actin, aortic smooth muscle |
| | ACTBL_HUMAN | Q562 | | 246 | | Beta-actin-like protein 2 |
| | ACTB_HUMAN | P60709 | | 245 | | Actin, cytoplasmic 1 |
| | ACTC_HUMAN | P68032 | | 247 | | Actin, alpha cardiac muscle 1 |
| | ACTG_HUMAN | P63261 | | 245 | | Actin, cytoplasmic 2 |
| | ACTH_HUMAN | P63267 | | 246 | | Actin, gamma-enteric |
| | | | | | | smooth muscle |
| | ACTK_HUMAN | Q9BYX7 | | 245 | | Kappa-actin |
| | ACTS_HUMAN | P68133 | | 247 | | Actin, alpha skeletal muscle |
| | A26CA_HUMAN | Q6S8J3 | | 923 | 3 | ANKRD26-like family C |
| | | | | | | member 1A |
| | ACTB_HUMAN | P60709 | | 223 | | Actin, cytoplasmic 1 |
| | ACTG_HUMAN | P63261 | | 223 | | Actin, cytoplasmic 2 |
| | AASD1_HUMAN | Q9BTE6 | | 81 | 1 | Alanyl-tRNA synthetase |
| | | | | | | domain-containing protein 1 |
| | ABL1_HUMAN | P00519 | | 940 | 1 | Proto-oncogene tyrosine- |
| | | | | | | protein kinase ABL1 |
| | ABLM1_HUMAN | O14639 | | 568 | 1 | Actin-binding LIM protein 1 |
| | ACAP3_HUMAN | Q96P50 | | 589 | 1 | ArfGAP with coiled-coil, |
| | | | | | | ANK repeat and PH domain- |
| | | | | | | containing protein 3 |
| | ACINU_HUMAN | Q9UKV3 | | 664 | 1 | Apoptotic chromatin |
| | | | | | | condensation inducer in the |
| | | | | | | nucleus |
| | ACINU_HUMAN | Q9UKV3 | | 512 | 1 | Apoptotic chromatin |
| | | | | | | condensation inducer in the |
| | | | | | | nucleus |
| | ACINU_HUMAN | Q9UKV3 | | 69 | 1 | Apoptotic chromatin |
| | | | | | | condensation inducer in the |
| | | | | | | nucleus |
| | ACINU_HUMAN | Q9UKV3 | | 664 | 1 | Apoptotic chromatin |
| | | | | | | condensation inducer in the |
| | | | | | | nucleus |
| | ACOC_HUMAN | P21399 | | 674 | 1 | Cytoplasmic aconitate |
| | | | | | | hydratase |
| | ACSL3_HUMAN | O95573 | | 572 | 2 | Long-chain-fatty-acid--CoA |
| | | O60488 | | | | ligase 3 |
| | ACSL4_HUMAN | | | 563 | | Long-chain-fatty-acid--CoA |
| | | | | | | ligase 4 |
| | ACTA_HUMAN | P62736 | | 54 | 6 | Actin, aortic smooth muscle |
| | ACTB_HUMAN | P60709 | | 52 | | Actin, cytoplasmic 1 |
| | ACTC_HUMAN | P68032 | | 54 | | Actin, alpha cardiac muscle 1 |
| | ACTG_HUMAN | P63261 | | 52 | | Actin, cytoplasmic 2 |
| | ACTH_HUMAN | P63267 | | 53 | | Actin, gamma-enteric |
| | | | | | | smooth muscle |
| | ACTS_HUMAN | P68133 | | 54 | | Actin, alpha skeletal muscle |
| | ACTA_HUMAN | P62736 | | 54 | 6 | Actin, aortic smooth muscle |
| | ACTB_HUMAN | P60709 | | 52 | | Actin, cytoplasmic 1 |
| | ACTC_HUMAN | P68032 | | 54 | | Actin, alpha cardiac muscle 1 |
| | ACTG_HUMAN | P63261 | | 52 | | Actin, cytoplasmic 2 |
| | ACTH_HUMAN | P63267 | | 53 | | Actin, gamma-enteric |
| | | | | | | smooth muscle |
| | ACTS_HUMAN | P68133 | | 54 | | Actin, alpha skeletal muscle |
| | ACTB_HUMAN | P60709 | | 155 | 2 | Actin, cytoplasmic 1 |
| | ACTG_HUMAN | P63261 | | 155 | | Actin, cytoplasmic 2 |
| | ACTB_HUMAN | P60709 | | 158 | 2 | Actin, cytoplasmic 1 |
| | ACTG_HUMAN | P63261 | | 158 | | Actin, cytoplasmic 2 |
| | ACTN1_HUMAN | P12814 | | 6 | 1 | Alpha-actinin-1 |
| | ACTN1_HUMAN | P12814 | | 6 | 1 | Alpha-actinin-1 |
| | ACTN1_HUMAN | P12814 | | 23 | 4 | Alpha-actinin-1 |
| | ACTN2_HUMAN | P35609 | | 30 | | Alpha-actinin-2 |
| | ACTN3_HUMAN | Q08043 | | 37 | | Alpha-actinin-3 |
| | ACTN4_HUMAN | O43707 | | 42 | | Alpha-actinin-4 |
| | ADDA_HUMAN | P35611 | | 634 | 1 | Alpha-adducin |
| | AEBP2_HUMAN | Q6ZN18 | | 234 | 1 | Zinc finger protein AEBP2 |
| | AEDO_HUMAN | Q96SZ5 | | 35 | 1 | 2-aminoethanethiol |
| | | | | | | dioxygenase |
| | AF1L2_HUMAN | Q8N4X5 | | 631 | 1 | Actin filament-associated |
| | | | | | | protein 1-like 2 |
| | AF1L2_HUMAN | Q8N4X5 | | 631 | 1 | Actin filament-associated |
| | | | | | | protein 1-like 2 |
| | AF1L2_HUMAN | Q8N4X5 | | 313 | 1 | Actin filament-associated |
| | | | | | | protein 1-like 2 |
| | AFTIN_HUMAN | Q6ULP2 | | 340 | 1 | Aftiphilin |
| | AGGF1_HUMAN | Q8N302 | | 149 | 1 | Angiogenic factor with G |
| | | | | | | patch and FHA domains 1 |
| | AGGF1_HUMAN | Q8N302 | | 149 | 1 | Angiogenic factor with G |
| | | | | | | patch and FHA domains 1 |
| | AGGF1_HUMAN | Q8N302 | | 149 | 1 | Angiogenic factor with G |
| | | | | | | patch and FHA domains 1 |
| | AHNK_HUMAN | Q09666 | | 3719 | 1 | Neuroblast differentiation- |
| | | | | | | associated protein AHNAK |
| | AHNK_HUMAN | Q09666 | | 1425 | 1 | Neuroblast differentiation- |
| | | | | | | associated protein AHNAK |
| | AHNK_HUMAN | Q09666 | | 2712 | 1 | Neuroblast differentiation- |
| | | | | | | associated protein AHNAK |
| | AHNK_HUMAN | Q09666 | | 3719 | 1 | Neuroblast differentiation- |
| | | | | | | associated protein AHNAK |
| | AHNK_HUMAN | Q09666 | | 5581 | 1 | Neuroblast differentiation- |
| | | | | | | associated protein AHNAK |
| | AHNK_HUMAN | Q09666 | | 576 | 1 | Neuroblast differentiation- |
| | | | | | | associated protein AHNAK |
| | AHNK_HUMAN | Q09666 | | 3494 | 1 | Neuroblast differentiation- |
| | | | | | | associated protein AHNAK |
| | AHNK_HUMAN | Q09666 | | 738 | 1 | Neuroblast differentiation- |
| | | | | | | associated protein AHNAK |
| | AHNK_HUMAN | Q09666 | | 866 | 1 | Neuroblast differentiation- |
| | | | | | | associated protein AHNAK |
| | AHNK_HUMAN | Q09666 | | 1584 | 1 | Neuroblast differentiation- |
| | | | | | | associated protein AHNAK |
| | AHNK_HUMAN | Q09666 | | 740 | 1 | Neuroblast differentiation- |
| | | | | | | associated protein AHNAK |
| | AHNK_HUMAN | Q09666 | | 3465 | 1 | Neuroblast differentiation- |
| | | | | | | associated protein AHNAK |
| | AHNK_HUMAN | Q09666 | | 920 | 1 | Neuroblast differentiation- |
| | | | | | | associated protein AHNAK |
| | AHNK_HUMAN | Q09666 | | 2883 | 1 | Neuroblast differentiation- |
| | | | | | | associated protein AHNAK |
| | AHNK_HUMAN | Q09666 | | 4359 | 1 | Neuroblast differentiation- |
| | | | | | | associated protein AHNAK |
| | AHNK_HUMAN | Q09666 | | 1169 | 1 | Neuroblast differentiation- |
| | | | | | | associated protein AHNAK |
| | AHSA1_HUMAN | O95433 | | 255 | 1 | Activator of 90 kDa heat |
| | | | | | | shock protein ATPase |
| | | | | | | homolog 1 |
| | AHSA1_HUMAN | O95433 | | 255 | 1 | Activator of 90 kDa heat |
| | | | | | | shock protein ATPase |
| | | | | | | homolog 1 |
| | AHSA1_HUMAN | O95433 | | 19 | 1 | Activator of 90 kDa heat |
| | | | | | | shock protein ATPase |
| | | | | | | homolog 1 |
| | AHTF1_HUMAN | Q8WYP5 | | 1368 | 1 | AT-hook-containing |
| | | | | | | transcription factor 1 |
| | AIM1_HUMAN | Q9Y4K1 | | 68 | 1 | Absent in melanoma 1 |
| | | | | | | protein |
| | AIM1_HUMAN | Q9Y4K1 | | 68 | 1 | Absent in melanoma 1 |
| | | | | | | protein |
| | AKA12_HUMAN | Q02952 | | 452 | 1 | A-kinase anchor protein 12 |
| | AKAP2_HUMAN | Q9Y2D5 | | 473 | 1 | A-kinase anchor protein 2 |
| | AKAP9_HUMAN | Q99996 | | 1034 | 1 | A-kinase anchor protein 9 |
| | AKAP9_HUMAN | Q99996 | | 1034 | 1 | A-kinase anchor protein 9 |
| | AKNA_HUMAN | Q7Z591 | | 800 | 1 | AT-hook-containing |
| | | | | | | transcription factor |
| | AKP13_HUMAN | Q12802 | | 545 | 1 | A-kinase anchor protein 13 |
| | AKP13_HUMAN | Q12802 | | 545 | 1 | A-kinase anchor protein 13 |
| | AKP13_HUMAN | Q12802 | | 830 | 1 | A-kinase anchor protein 13 |
| | AKP13_HUMAN | Q12802 | | 906 | 1 | A-kinase anchor protein 13 |
| | AKP13_HUMAN | Q12802 | | 1056 | 1 | A-kinase anchor protein 13 |
| | AKP13_HUMAN | Q12802 | | 1540 | 1 | A-kinase anchor protein 13 |
| | AKP8L_HUMAN | Q9ULX6 | | 109 | 1 | A-kinase anchor protein 8- |
| | | | | | | like |
| | ALMS1_HUMAN | Q8TCU4 | | 428 | 1 | Alstrom syndrome protein 1 |
| | ALMS1_HUMAN | Q8TCU4 | | 780 | 1 | Alstrom syndrome protein 1 |
| | ALMS1_HUMAN | Q8TCU4 | | 591 | 1 | Alstrom syndrome protein 1 |
| 2 | ALO17_HUMAN | Q9HCF4 | AVAEPANAVK | 274 | 1 | Protein ALO17 |
| 3 | ALO17_HUMAN | Q9HCF4 | AVAEPANAVKGA | 274 | 1 | Protein ALO17 |
| | | | GK | | | |
| 4 | ALO17_HUMAN | Q9HCF4 | AVAEPANAVKGA | 274 | 1 | Protein ALO17 |
| | | | GKEMK | | | |
| | AMPD3_HUMAN | Q01432 | | 37 | 1 | AMP deaminase 3 |
| | AMPM1_HUMAN | P53582 | | 13 | 1 | Methionine aminopeptidase 1 |
| | ANKH1_HUMAN | Q8IWZ3 | | 1049 | 1 | Ankyrin repeat and KH |
| | | | | | | domain-containing protein 1 |
| | ANKH1_HUMAN | Q8IWZ3 | | 5 | 1 | Ankyrin repeat and KH |
| | | | | | | domain-containing protein 1 |
| | ANKS6_HUMAN | Q68DC2 | | 276 | 1 | Ankyrin repeat and SAM |
| | | | | | | domain-containing protein 6 |
| | ANS1A_HUMAN | Q92625 | | 530 | 1 | Ankyrin repeat and SAM |
| | | | | | | domain-containing protein |
| | | | | | | 1A |
| | ANXA2_HUMAN | P07355 | | 17 | 2 | Annexin A2 |
| | AXA2L_HUMAN | A6NMY6 | | 17 | | Putative annexin A2-like |
| | | | | | | protein |
| | AP1G1_HUMAN | O43747 | | 690 | 1 | AP-1 complex subunit |
| | | | | | | gamma-1 |
| | AP1G2_HUMAN | O75843 | | 632 | 1 | AP-1 complex subunit |
| | | | | | | gamma-like 2 |
| | AP2A2_HUMAN | O94973 | | 691 | 1 | AP-2 complex subunit alpha-2 |
| | AP3B2_HUMAN | Q13367 | | 844 | 1 | AP-3 complex subunit beta-2 |
| | AP3B2_HUMAN | Q13367 | | 844 | 1 | AP-3 complex subunit beta-2 |
| | APBB2_HUMAN | Q92870 | | 280 | 1 | Amyloid beta A4 precursor |
| | | | | | | protein-binding family B |
| | | | | | | member 2 |
| | APC_HUMAN | P25054 | | 1499 | 1 | Adenomatous polyposis coli |
| | | | | | | protein |
| | APMAP_HUMAN | Q9HDC9 | | 23 | 1 | Adipocyte plasma |
| | | | | | | membrane-associated protein |
| | APTX_HUMAN | Q7Z2E3 | | 142 | 1 | Aprataxin |
| | AR13B_HUMAN | Q3SXY8 | | 242 | 1 | ADP-ribosylation factor-like |
| | | | | | | protein 13B |
| | ARBK1_HUMAN | P25098 | | 528 | 1 | Beta-adrenergic receptor |
| | | | | | | kinase 1 |
| | ARBK1_HUMAN | P25098 | | 482 | 2 | Beta-adrenergic receptor |
| | | | | | | kinase 1 |
| | ARBK2_HUMAN | P35626 | | 482 | | Beta-adrenergic receptor |
| | | | | | | kinase 2 |
| | ARBK1_HUMAN | P25098 | | 482 | 2 | Beta-adrenergic receptor |
| | | | | | | kinase 1 |
| | ARBK2_HUMAN | P35626 | | 482 | | Beta-adrenergic receptor |
| | | | | | | kinase 2 |
| | ARHG1_HUMAN | Q92888 | | 293 | 1 | Rho guanine nucleotide |
| | | | | | | exchange factor 1 |
| | ARHG2_HUMAN | Q92974 | | 627 | 1 | Rho guanine nucleotide |
| | | | | | | exchange factor 2 |
| | ARHGA_HUMAN | O15013 | | 1247 | 1 | Rho guanine nucleotide |
| | | | | | | exchange factor 10 |
| | ARI1A_HUMAN | O14497 | | 607 | 1 | AT-rich interactive domain- |
| | | | | | | containing protein 1A |
| | ARI1A_HUMAN | O14497 | | 607 | 1 | AT-rich interactive domain- |
| | | | | | | containing protein 1A |
| | ARI1A_HUMAN | O14497 | | 76 | 1 | AT-rich interactive domain- |
| | | | | | | containing protein 1A |
| | ARI4A_HUMAN | P29374 | | 1031 | 1 | AT-rich interactive domain- |
| | | | | | | containing protein 4A |
| | ARI4B_HUMAN | Q4LE39 | | 1073 | 1 | AT-rich interactive domain- |
| | | | | | | containing protein 4B |
| | ARID2_HUMAN | Q68CP9 | | 626 | 1 | AT-rich interactive domain- |
| | | | | | | containing protein 2 |
| | ARID2_HUMAN | Q68CP9 | | 630 | 1 | AT-rich interactive domain- |
| | | | | | | containing protein 2 |
| | ARM10_HUMAN | Q8N2F6 | | 87 | 1 | Armadillo repeat-containing |
| | | | | | | protein 10 |
| | ARMC6_HUMAN | Q6NXE6 | | 83 | 1 | Armadillo repeat-containing |
| | | | | | | protein 6 |
| | ARMC6_HUMAN | Q6NXE6 | | 83 | 1 | Armadillo repeat-containing |
| | | | | | | protein 6 |
| | ARNT_HUMAN | P27540 | | 152 | 1 | Aryl hydrocarbon receptor |
| | | | | | | nuclear translocator |
| | ARP21_HUMAN | Q9UBL0 | | 495 | 1 | cAMP-regulated |
| | | | | | | phosphoprotein 21 |
| | ARP2_HUMAN | P61160 | | 162 | 1 | Actin-related protein 2 |
| | ARP3_HUMAN | P61158 | | 60 | 1 | Actin-related protein 3 |
| | ARPC5_HUMAN | O15511 | | 30 | 1 | Actin-related protein 2/3 |
| | | | | | | complex subunit 5 |
| | ARPC5_HUMAN | O15511 | | 33 | 1 | Actin-related protein 2/3 |
| | | | | | | complex subunit 5 |
| | ARS2_HUMAN | Q9BXP5 | | 162 | 1 | Arsenite-resistance protein 2 |
| | ASB13_HUMAN | Q8WXK3 | | 52 | 1 | Ankyrin repeat and SOCS |
| | | | | | | box protein 13 |
| | ASCC1_HUMAN | Q8N9N2 | | 35 | 1 | Activating signal |
| | | | | | | cointegrator 1 complex |
| | | | | | | subunit 1 |
| | ASCC2_HUMAN | Q9H1I8 | | 622 | 1 | Activating signal |
| | | | | | | cointegrator 1 complex |
| | | | | | | subunit 2 |
| | ASHWN_HUMAN | Q9BVC5 | | 106 | 1 | Ashwin |
| | ASPP2_HUMAN | Q13625 | | 528 | 1 | Apoptosis-stimulating of p53 |
| | | | | | | protein 2 |
| | ATAD5_HUMAN | Q96QE3 | | 285 | 1 | ATPase family AAA |
| | | | | | | domain-containing protein 5 |
| | ATD2B_HUMAN | Q9ULI0 | | 78 | 1 | ATPase family AAA |
| | | | | | | domain-containing protein |
| | | | | | | 2B |
| | ATF1_HUMAN | P18846 | | 47 | 1 | Cyclic AMP-dependent |
| | | | | | | transcription factor ATF-1 |
| 5 | ATF4_HUMAN | P18848 | GLVSPSNNSKEDA | 66 | 1 | Cyclic AMP-dependent |
| | | | FSGTDWMLEK | | | transcription factor ATF-4 |
| | ATF7_HUMAN | P17544 | | 44 | 1 | Cyclic AMP-dependent |
| | | | | | | transcription factor ATF-7 |
| | ATF7_HUMAN | P17544 | | 44 | 1 | Cyclic AMP-dependent |
| | | | | | | transcription factor ATF-7 |
| | ATG3_HUMAN | Q9NT62 | | 105 | 1 | Autophagy-related protein 3 |
| | ATG3_HUMAN | Q9NT62 | | 105 | 1 | Autophagy-related protein 3 |
| | ATG4B_HUMAN | Q9Y4P1 | | 3 | 1 | Cysteine protease ATG4B |
| | ATRX_HUMAN | P46100 | | 920 | 1 | Transcriptional regulator |
| | | | | | | ATRX |
| | ATX1L_HUMAN | P0C7T5 | | 309 | 1 | Ataxin-1-like |
| | ATX2L_HUMAN | Q8WWM7 | | 585 | 1 | Ataxin-2-like protein |
| | ATX2L_HUMAN | Q8WWM7 | | 585 | 1 | Ataxin-2-like protein |
| | ATX2_HUMAN | Q99700 | | 843 | 1 | Ataxin-2 |
| 6 | ATX3_HUMAN | P54252 | GSGMLDEDEEDL | 218 | 1 | Ataxin-3 |
| | | | QR | | | |
| | AZI1_HUMAN | Q9UPN4 | | 549 | 1 | 5-azacytidine-induced |
| | | | | | | protein 1 |
| | BA2D1_HUMAN | Q9Y520 | | 889 | 1 | BAT2 domain-containing |
| | | | | | | protein 1 |
| | BA2D1_HUMAN | Q9Y520 | | 2190 | 1 | BAT2 domain-containing |
| | | | | | | protein 1 |
| | BAP1_HUMAN | Q92560 | | 312 | 1 | Ubiquitin carboxyl-terminal |
| | | | | | | hydrolase BAP1 |
| | BAP31_HUMAN | P51572 | | 165 | 1 | B-cell receptor-associated |
| | | | | | | protein 31 |
| | BAP31_HUMAN | P51572 | | 165 | 1 | B-cell receptor-associated |
| | | | | | | protein 31 |
| | BASP_HUMAN | P80723 | | 166 | 1 | Brain acid soluble protein 1 |
| | BASP_HUMAN | P80723 | | 172 | 1 | Brain acid soluble protein 1 |
| | BAT3_HUMAN | P46379 | | 1002 | 1 | Large proline-rich protein |
| | | | | | | BAT3 |
| | BAT3_HUMAN | P46379 | | 1002 | 1 | Large proline-rich protein |
| | | | | | | BAT3 |
| | BAZ1A_HUMAN | Q9NRL2 | | 500 | 1 | Bromodomain adjacent to |
| | | | | | | zinc finger domain protein |
| | | | | | | 1A |
| 7 | BCAP_HUMAN | Q6ZUJ8 | SVTDTEPEDEK | 149 | 1 | Phosphoinositide 3-kinase |
| | | | | | | adapter protein 1 |
| 8 | BCAP_HUMAN | Q6ZUJ8 | SVTDTEPEDEKVV | 149 | 1 | Phosphoinositide 3-kinase |
| | | | SYSK | | | adapter protein 1 |
| | BCLF1_HUMAN | Q9NYF8 | | 325 | 1 | Bcl-2-associated |
| | | | | | | transcription factor 1 |
| | BCLF1_HUMAN | Q9NYF8 | | 383 | 1 | Bcl-2-associated |
| | | | | | | transcription factor 1 |
| | BCR_HUMAN | P11274 | | 244 | 1 | Breakpoint cluster region |
| | | | | | | protein |
| | BDP1_HUMAN | A6H8Y1 | | 526 | 1 | Transcription factor TFIIIB |
| | | | | | | component B″ homolog |
| | BID_HUMAN | P55957 | | 76 | 1 | BH3-interacting domain |
| | | | | | | death agonist |
| | BIG3_HUMAN | Q5TH69 | | 293 | 1 | Brefeldin A-inhibited |
| | | | | | | guanine nucleotide-exchange |
| | | | | | | protein 3 |
| | BIN1_HUMAN | O00499 | | 302 | 1 | Myc box-dependent- |
| | | | | | | interacting protein 1 |
| | BIRC6_HUMAN | Q9NR09 | | 462 | 1 | Baculoviral IAP repeat- |
| | | | | | | containing protein 6 |
| | BL1S3_HUMAN | Q6QNY0 | | 65 | 1 | Biogenesis of lysosome- |
| | | | | | | related organelles complex 1 |
| | | | | | | subunit 3 |
| 9 | BLNK_HUMAN | Q8WV28 | YVVPVEDNDENY | 178 | 1 | B-cell linker protein |
| | | | IHPTESSSPPPEK | | | |
| | BNIP2_HUMAN | Q12982 | | 84 | 1 | BCL2/adenovirus E1B 19 kDa |
| | | | | | | protein-interacting |
| | | | | | | protein 2 |
| | BPTF_HUMAN | Q12830 | | 1626 | 1 | Nucleosome-remodeling |
| | | | | | | factor subunit BPTF |
| | BRD1_HUMAN | O95696 | | 922 | 1 | Bromodomain-containing |
| | | | | | | protein 1 |
| | BRD4_HUMAN | O60885 | | 338 | 1 | Bromodomain-containing |
| | | | | | | protein 4 |
| | BRD8_HUMAN | Q9H0E9 | | 561 | 1 | Bromodomain-containing |
| | | | | | | protein 8 |
| | BTB14_HUMAN | Q96RE7 | | 175 | 1 | BTB/POZ domain- |
| | | | | | | containing protein 14B |
| | BUB1_HUMAN | O43683 | | 396 | 1 | Mitotic checkpoint |
| | | | | | | serine/threonine-protein |
| | | | | | | kinase BUB1 |
| | BUB1_HUMAN | O43683 | | 396 | 1 | Mitotic checkpoint |
| | | | | | | serine/threonine-protein |
| | | | | | | kinase BUB1 |
| | BUD13_HUMAN | Q9BRD0 | | 274 | 1 | BUD13 homolog |
| | C170L_HUMAN | Q96L14 | | 51 | 1 | Cep170-like protein |
| | C1QBP_HUMAN | Q07021 | | 186 | 1 | Complement component 1 Q |
| | | | | | | subcomponent-binding |
| | | | | | | protein, mitochondrial |
| | C2C2L_HUMAN | O14523 | | 443 | 1 | C2 domain-containing |
| | | | | | | protein 2-like |
| | C2D1A_HUMAN | Q6P1N0 | | 31 | 1 | Coiled-coil and C2 domain- |
| | | | | | | containing protein 1A |
| | C2D1A_HUMAN | Q6P1N0 | | 31 | 1 | Coiled-coil and C2 domain- |
| | | | | | | containing protein 1A |
| | C2D1B_HUMAN | Q5T0F9 | | 461 | 1 | Coiled-coil and C2 domain- |
| | | | | | | containing protein 1B |
| | CA059_HUMAN | Q5T8I9 | | 14 | 1 | UPF0486 protein C1orf59 |
| | CA059_HUMAN | Q5T8I9 | | 14 | 1 | UPF0486 protein C1orf59 |
| | CA103_HUMAN | Q5T3J3 | | 516 | 1 | Uncharacterized protein |
| | | | | | | C1orf103 |
| | CA103_HUMAN | Q5T3J3 | | 516 | 1 | Uncharacterized protein |
| | | | | | | C1orf103 |
| | CA163_HUMAN | Q96BR5 | | 121 | 1 | Hcp beta-lactamase-like |
| | | | | | | protein C1orf163 |
| | CA165_HUMAN | Q7L4P6 | | 104 | 1 | Coiled-coil domain- |
| | | | | | | containing protein C1orf165 |
| | CA170_HUMAN | Q5SV97 | | 43 | 1 | Uncharacterized protein |
| | | | | | | C1orf170 |
| | CA175_HUMAN | Q68CQ1 | | 412 | 1 | Uncharacterized protein |
| | | | | | | C1orf175 |
| | CA1L1_HUMAN | Q08AD1 | | 422 | 1 | Calmodulin-regulated |
| | | | | | | spectrin-associated protein 1- |
| | | | | | | like protein 1 |
| | CABL2_HUMAN | Q9BTV7 | | 59 | 1 | CDK5 and ABL1 enzyme |
| | | | | | | substrate 2 |
| | CACO1_HUMAN | Q9P1Z2 | | 135 | 1 | Calcium-binding and coiled- |
| | | | | | | coil domain-containing |
| | | | | | | protein 1 |
| | CADH2_HUMAN | P19022 | | 800 | 1 | Cadherin-2 |
| | CADH2_HUMAN | P19022 | | 800 | 1 | Cadherin-2 |
| | CAF1A_HUMAN | Q13111 | | 615 | 1 | Chromatin assembly factor 1 |
| | | | | | | subunit A |
| | CAF1A_HUMAN | Q13111 | | 111 | 1 | Chromatin assembly factor 1 |
| | | | | | | subunit A |
| | CAF1A_HUMAN | Q13111 | | 111 | 1 | Chromatin assembly factor 1 |
| | | | | | | subunit A |
| | CALR_HUMAN | P27797 | | 259 | 1 | Calreticulin |
| | CALR_HUMAN | P27797 | | 329 | 1 | Calreticulin |
| 10 | CALR_HUMAN | P27797 | MHGDSEYNIMFG | 122 | 1 | Calreticulin |
| | | | PDICGPGTK | | | |
| | CAMKV_HUMAN | Q8NCB2 | | 408 | 1 | CaM kinase-like vesicle- |
| | | | | | | associated protein |
| | CAMLG_HUMAN | P49069 | | 10 | 1 | Calcium signal-modulating |
| | | | | | | cyclophilin ligand |
| | CAMLG_HUMAN | P49069 | | 10 | 1 | Calcium signal-modulating |
| | | | | | | cyclophilin ligand |
| | CAMLG_HUMAN | P49069 | | 116 | 1 | Calcium signal-modulating |
| | | | | | | cyclophilin ligand |
| | CAMP1_HUMAN | Q5T5Y3 | | 752 | 1 | Calmodulin-regulated |
| | | | | | | spectrin-associated protein 1 |
| | CAMP1_HUMAN | Q5T5Y3 | | 1255 | 1 | Calmodulin-regulated |
| | | | | | | spectrin-associated protein 1 |
| | CAMP1_HUMAN | Q5T5Y3 | | 1255 | 1 | Calmodulin-regulated |
| | | | | | | spectrin-associated protein 1 |
| | CAPR1_HUMAN | Q14444 | | 95 | 1 | Caprin-1 |
| | CAPZB_HUMAN | P47756 | | 150 | 1 | F-actin-capping protein |
| | | | | | | subunit beta |
| | CASC3_HUMAN | O15234 | | 390 | 1 | Protein CASC3 |
| | CASC5_HUMAN | Q8NG31 | | 1195 | 1 | Protein CASC5 |
| | CASP3_HUMAN | P42574 | | 29 | 1 | Caspase-3 |
| | CASP3_HUMAN | P42574 | | 176 | 1 | Caspase-3 |
| | CASP3_HUMAN | P42574 | | 176 | 1 | Caspase-3 |
| | CASP7_HUMAN | P55210 | | 199 | 1 | Caspase-7 |
| | CASP_HUMAN | Q13948 | | 388 | 2 | Protein CASP |
| | CUX1_HUMAN | P39880 | | 377 | | Homeobox protein cut-like 1 |
| | CATB_HUMAN | P07858 | | 78 | 1 | Cathepsin B |
| | CB044_HUMAN | Q9H6R7 | | 509 | 1 | WD repeat-containing |
| | | | | | | protein C2orf44 |
| | CBL_HUMAN | P22681 | | 807 | 1 | E3 ubiquitin-protein ligase |
| | | | | | | CBL |
| | CBWD1_HUMAN | Q9BRT8 | | 185 | 6 | COBW domain-containing |
| | | | | | | protein 1 |
| | CBWD2_HUMAN | Q8IUF1 | | 185 | | COBW domain-containing |
| | | | | | | protein 2 |
| | CBWD3_HUMAN | Q5JTY5 | | 185 | | COBW domain-containing |
| | | | | | | protein 3 |
| | CBWD5_HUMAN | Q5RIA9 | | 185 | | COBW domain-containing |
| | | | | | | protein 5 |
| | CBWD6_HUMAN | Q4V339 | | 185 | | COBW domain-containing |
| | | | | | | protein 6 |
| | CBWD7_HUMAN | A6NM15 | | 37 | | COBW domain-containing |
| | | | | | | protein 7 |
| | CC104_HUMAN | Q96G28 | | 142 | 1 | Coiled-coil domain- |
| | | | | | | containing protein 104 |
| 11 | CC104_HUMAN | Q96G28 | GSDVVSDLEHEE | 142 | 1 | Coiled-coil domain- |
| | | | MK | | | containing protein 104 |
| | CC104_HUMAN | Q96G28 | | 145 | 1 | Coiled-coil domain- |
| | | | | | | containing protein 104 |
| | CC104_HUMAN | Q96G28 | | 145 | 1 | Coiled-coil domain- |
| | | | | | | containing protein 104 |
| | CC124_HUMAN | Q96CT7 | | 150 | 1 | Coiled-coil domain- |
| | | | | | | containing protein 124 |
| | CC131_HUMAN | O60293 | | 336 | 1 | Coiled-coil domain- |
| | | | | | | containing protein 131 |
| 12 | CC50A_HUMAN | Q9NV96 | GGPPCAPGGTAK | 13 | 1 | Cell cycle control protein |
| | | | | | | 50A |
| | CCD43_HUMAN | Q96MW1 | | 17 | 1 | Coiled-coil domain- |
| | | | | | | containing protein 43 |
| | CCD53_HUMAN | Q9Y3C0 | | 5 | 1 | Coiled-coil domain- |
| | | | | | | containing protein 53 |
| | CCD91_HUMAN | Q7Z6B0 | | 100 | 1 | Coiled-coil domain- |
| | | | | | | containing protein 91 |
| | CCD97_HUMAN | Q96F63 | | 53 | 1 | Coiled-coil domain- |
| | | | | | | containing protein 97 |
| | CCDC9_HUMAN | Q9Y3X0 | | 300 | 1 | Coiled-coil domain- |
| | | | | | | containing protein 9 |
| | CCDC9_HUMAN | Q9Y3X0 | | 300 | 1 | Coiled-coil domain- |
| | | | | | | containing protein 9 |
| | CCNT2_HUMAN | O60583 | | 455 | 1 | Cyclin-T2 |
| | CCNT2_HUMAN | O60583 | | 455 | 1 | Cyclin-T2 |
| | CD2L1_HUMAN | P21127 | | 406 | 1 | PITSLRE serine/threonine- |
| | | | | | | protein kinase CDC2L1 |
| | CD2L1_HUMAN | P21127 | | 406 | 1 | PITSLRE serine/threonine- |
| | | | | | | protein kinase CDC2L1 |
| | CD2L5_HUMAN | Q14004 | | 1354 | 1 | Cell division cycle 2-like |
| | | | | | | protein kinase 5 |
| | CDC27_HUMAN | P30260 | | 237 | 1 | Cell division cycle protein 27 |
| | | | | | | homolog |
| | CDC27_HUMAN | P30260 | | 244 | 1 | Cell division cycle protein 27 |
| | | | | | | homolog |
| | CDC5L_HUMAN | Q99459 | | 392 | 1 | Cell division cycle 5-like |
| | | | | | | protein |
| | CDCA7_HUMAN | Q9BWT1 | | 40 | 1 | Cell division cycle- |
| | | | | | | associated protein 7 |
| | CDV3_HUMAN | Q9UKY7 | | 123 | 1 | Protein CDV3 homolog |
| 13 | CDYL1_HUMAN | Q9Y232 | GFQSESPEKLDPV | 211 | 1 | Chromodomain Y-like |
| | | | EQGQEDTVAPEV | | | protein |
| | | | AAEKPVGALLGP | | | |
| | | | GAER | | | |
| | CE022_HUMAN | Q49AR2 | | 197 | 1 | UPF0489 protein C5orf22 |
| | CE152_HUMAN | O94986 | | 63 | 1 | Centrosomal protein of 152 kDa |
| | CE170_HUMAN | Q5SW79 | | 1325 | 1 | Centrosomal protein of 170 kDa |
| | CE170_HUMAN | Q5SW79 | | 1325 | 1 | Centrosomal protein of 170 kDa |
| | CE170_HUMAN | Q5SW79 | | 937 | 1 | Centrosomal protein of 170 kDa |
| | CEBPZ_HUMAN | Q03701 | | 918 | 1 | CCAAT/enhancer-binding |
| | | | | | | protein zeta |
| | CEBPZ_HUMAN | Q03701 | | 775 | 1 | CCAAT/enhancer-binding |
| | | | | | | protein zeta |
| | CEBPZ_HUMAN | Q03701 | | 956 | 1 | CCAAT/enhancer-binding |
| | | | | | | protein zeta |
| | CH041_HUMAN | Q6NXR4 | | 5 | 1 | Uncharacterized protein |
| | | | | | | C8orf41 |
| | CH082_HUMAN | Q6P1X6 | | 26 | 1 | UPF0598 protein C8orf82 |
| | CH60_HUMAN | P10809 | | 505 | 1 | 60 kDa heat shock protein, |
| | | | | | | mitochondrial |
| | CH60_HUMAN | P10809 | | 112 | 1 | 60 kDa heat shock protein, |
| | | | | | | mitochondrial |
| | CH60_HUMAN | P10809 | | 453 | 1 | 60 kDa heat shock protein, |
| | | | | | | mitochondrial |
| | CH60_HUMAN | P10809 | | 453 | 1 | 60 kDa heat shock protein, |
| | | | | | | mitochondrial |
| | CHD3_HUMAN | Q12873 | | 373 | 3 | Chromodomain-helicase- |
| | | | | | | DNA-binding protein 3 |
| | CHD4_HUMAN | Q14839 | | 364 | | Chromodomain-helicase- |
| | | | | | | DNA-binding protein 4 |
| | CHD5_HUMAN | Q8TDI0 | | 337 | | Chromodomain-helicase- |
| | | | | | | DNA-binding protein 5 |
| 14 | CHD4_HUMAN | Q14839 | GGGDNKEGEDSS | 1234 | 1 | Chromodomain-helicase- |
| | | | VIHYDDK | | | DNA-binding protein 4 |
| 15 | CHD4_HUMAN | Q14839 | GGGDNKEGEDSS | 1234 | 1 | Chromodomain-helicase- |
| | | | VIHYDDKAIER | | | DNA-binding protein 4 |
| 16 | CHD7_HUMAN | Q9P2D1 | GFYMEDGDPSVA | 2286 | 1 | Chromodomain-helicase- |
| | | | QLLHER | | | DNA-binding protein 7 |
| 17 | CHM4B_HUMAN | Q9H444 | GTLSTIEFQR | 84 | 3 | Charged multivesicular body |
| | | | | | | protein 4b |
| 17 | CHM4C_HUMAN | Q96CF2 | GTLSTIEFQR | 84 | | Charged multivesicular body |
| | | | | | | protein 4c |
| | CI080_HUMAN | Q9NRY2 | | 58 | 1 | Uncharacterized protein |
| | | | | | | C9orf80 |
| | CJ018_HUMAN | Q5VWN6 | | 1208 | 1 | Uncharacterized protein |
| | | | | | | C10orf18 |
| | CJ047_HUMAN | Q86WR7 | | 110 | 1 | Uncharacterized protein |
| | | | | | | C10orf47 |
| | CK059_HUMAN | Q6IAA8 | | 73 | 1 | UPF0404 protein C11orf59 |
| | CK059_HUMAN | Q6IAA8 | | 73 | 1 | UPF0404 protein C11orf59 |
| | CL035_HUMAN | Q9HCM1 | | 360 | 1 | Uncharacterized protein |
| | | | | | | C12orf35 |
| | CL035_HUMAN | Q9HCM1 | | 502 | 1 | Uncharacterized protein |
| | | | | | | C12orf35 |
| | CL043_HUMAN | Q96C57 | | 73 | 1 | Uncharacterized protein |
| | | | | | | C12orf43 |
| | CL043_HUMAN | Q96C57 | | 205 | 1 | Uncharacterized protein |
| | | | | | | C12orf43 |
| | CL043_HUMAN | Q96C57 | | 205 | 1 | Uncharacterized protein |
| | | | | | | C12orf43 |
| | CL043_HUMAN | Q96C57 | | 205 | 1 | Uncharacterized protein |
| | | | | | | C12orf43 |
| | CLAP1_HUMAN | Q7Z460 | | 1219 | 1 | CLIP-associating protein 1 |
| | CLAP1_HUMAN | Q7Z460 | | 1219 | 1 | CLIP-associating protein 1 |
| | CLCA_HUMAN | P09496 | | 77 | 1 | Clathrin light chain A |
| | CLCA_HUMAN | P09496 | | 77 | 1 | Clathrin light chain A |
| | CLCA_HUMAN | P09496 | | 93 | 1 | Clathrin light chain A |
| | CLIC1_HUMAN | O00299 | | 142 | 1 | Chloride intracellular |
| | | | | | | channel protein 1 |
| | CLIP1_HUMAN | P30622 | | 398 | 1 | CAP-Gly domain-containing |
| | | | | | | linker protein 1 |
| | CLSPN_HUMAN | Q9HAW4 | | 564 | 1 | Claspin |
| | CND2_HUMAN | Q15003 | | 200 | 1 | Condensin complex subunit 2 |
| 18 | CND2_HUMAN | Q15003 | GSLGDDFDANDE | 367 | 1 | Condensin complex subunit 2 |
| | | | PDHTAVGDHEEFR | | | |
| | CND2_HUMAN | Q15003 | | 381 | 1 | Condensin complex subunit 2 |
| | CND2_HUMAN | Q15003 | | 171 | 1 | Condensin complex subunit 2 |
| | CND2_HUMAN | Q15003 | | 200 | 1 | Condensin complex subunit 2 |
| | CNDH2_HUMAN | Q6IBW4 | | 460 | 1 | Condensin-2 complex |
| | | | | | | subunit H2 |
| | CO6A3_HUMAN | P12111 | | 2616 | 1 | Collagen alpha-3(VI) chain |
| | COBL1_HUMAN | Q53SF7 | | 984 | 1 | Cordon-bleu protein-like 1 |
| 19 | COPA_HUMAN | P53621 | GFVEATEGLGDD | 857 | 1 | Coatomer subunit alpha |
| | | | ALGK | | | |
| 20 | COPA_HUMAN | P53621 | LFGTTDAVVK | 189 | 1 | Coatomer subunit alpha |
| | COPB2_HUMAN | P35606 | | 855 | 1 | Coatomer subunit beta′ |
| | COR1A_HUMAN | P31146 | | 395 | 2 | Coronin-1A |
| | CP088_HUMAN | Q1ED39 | | 183 | 1 | Protein C16orf88 |
| | CP110_HUMAN | Q7Z7A1 | | 1396 | 1 | Centriolin |
| | CP110_HUMAN | Q7Z7A1 | | 802 | 1 | Centriolin |
| | CPIN1_HUMAN | Q6FI81 | | 215 | 1 | Anamorsin |
| | CPNE1_HUMAN | Q99829 | | 465 | 1 | Copine-1 |
| | CPNE3_HUMAN | O75131 | | 429 | 1 | Copine-3 |
| | CPSF6_HUMAN | Q16630 | | 55 | 1 | Cleavage and |
| | | | | | | polyadenylation specificity |
| | | | | | | factor subunit 6 |
| | CPSF7_HUMAN | Q8N684 | | 325 | 1 | Cleavage and |
| | | | | | | polyadenylation specificity |
| | | | | | | factor subunit 7 |
| | CPSF7_HUMAN | Q8N684 | | 30 | 1 | Cleavage and |
| | | | | | | polyadenylation specificity |
| | | | | | | factor subunit 7 |
| | CPSF7_HUMAN | Q8N684 | | 34 | 1 | Cleavage and |
| | | | | | | polyadenylation specificity |
| | | | | | | factor subunit 7 |
| | CPZIP_HUMAN | Q6JBY9 | | 273 | 1 | Capz-interacting protein |
| | CQ056_HUMAN | Q96N21 | | 381 | 1 | Uncharacterized protein |
| | | | | | | C17orf56 |
| | CQ085_HUMAN | Q53F19 | | 158 | 1 | Uncharacterized protein |
| | | | | | | C17orf85 |
| | CQ085_HUMAN | Q53F19 | | 232 | 1 | Uncharacterized protein |
| | | | | | | C17orf85 |
| | CR025_HUMAN | Q96B23 | | 45 | 1 | Uncharacterized protein |
| | | | | | | C18orf25 |
| | CR025_HUMAN | Q96B23 | | 45 | 1 | Uncharacterized protein |
| | | | | | | C18orf25 |
| | CR025_HUMAN | Q96B23 | | 45 | 1 | Uncharacterized protein |
| | | | | | | C18orf25 |
| 21 | CR025_HUMAN | Q96B23 | GVADSTVISSMPC | 45 | 1 | Uncharacterized protein |
| | | | LLMELR | | | C18orf25 |
| 22 | CR025_HUMAN | Q96B23 | GVADSTVISSMPC | 45 | 1 | Uncharacterized protein |
| | | | LLMELRR | | | C18orf25 |
| | CREB1_HUMAN | P16220 | | 230 | 1 | cAMP response element- |
| | | | | | | binding protein |
| | CREB1_HUMAN | P16220 | | 117 | 1 | cAMP response element- |
| | | | | | | binding protein |
| | CREB1_HUMAN | P16220 | | 117 | 1 | cAMP response element- |
| | | | | | | binding protein |
| | CROCC_HUMAN | Q5TZA2 | | 579 | 1 | Rootletin |
| | CS043_HUMAN | Q9BQ61 | | 63 | 1 | Uncharacterized protein |
| | | | | | | C19orf43 |
| | CS044_HUMAN | Q9H6X5 | | 369 | 1 | Uncharacterized protein |
| | | | | | | C19orf44 |
| | CSN1_HUMAN | Q13098 | | 95 | 1 | COP9 signalosome complex |
| | | | | | | subunit 1 |
| | CSRN2_HUMAN | Q9H175 | | 40 | 1 | Cysteine/serine-rich nuclear |
| | | | | | | protein 2 |
| | CSTF3_HUMAN | Q12996 | | 577 | 1 | Cleavage stimulation factor |
| | | | | | | 77 kDa subunit |
| | CTBL1_HUMAN | Q8WYA6 | | 67 | 1 | Beta-catenin-like protein 1 |
| | CTCF_HUMAN | P49711 | | 47 | 1 | Transcriptional repressor |
| | | | | | | CTCF |
| | CTCF_HUMAN | P49711 | | 47 | 1 | Transcriptional repressor |
| | | | | | | CTCF |
| | CTCF_HUMAN | P49711 | | 47 | 1 | Transcriptional repressor |
| | | | | | | CTCF |
| | CTNB1_HUMAN | P35222 | | 116 | 1 | Catenin beta-1 |
| | CTND1_HUMAN | O60716 | | 162 | 1 | Catenin delta-1 |
| | CTR9_HUMAN | Q6PD62 | | 1121 | 1 | RNA polymerase-associated |
| | | | | | | protein CTR9 homolog |
| | CUL4B_HUMAN | Q13620 | | 26 | 1 | Cullin-4B |
| | CUTC_HUMAN | Q9NTM9 | | 34 | 1 | Copper homeostasis protein |
| | | | | | | cutC homolog |
| | CUX1_HUMAN | P39880 | | 1340 | 1 | Homeobox protein cut-like 1 |
| 23 | CYB5B_HUMAN | O43169 | GKGQEVETSVTY | 11 | 1 | Cytochrome b5 type B |
| | | | YR | | | |
| | DBPA_HUMAN | P16989 | | 270 | 1 | DNA-binding protein A |
| | DBPA_HUMAN | P16989 | | 162 | 1 | DNA-binding protein A |
| | DBPA_HUMAN | P16989 | | 145 | 1 | DNA-binding protein A |
| | DBPA_HUMAN | P16989 | | 138 | 3 | DNA-binding protein A |
| | YBOX1_HUMAN | P67809 | | 106 | | Nuclease-sensitive element- |
| | | | | | | binding protein 1 |
| | YBOX2_HUMAN | Q9Y2T7 | | 141 | | Y-box-binding protein 2 |
| | DCNL2_HUMAN | Q6PH85 | | 43 | 1 | DCN1-like protein 2 |
| | DCTN1_HUMAN | Q14203 | | 303 | 1 | Dynactin subunit 1 |
| | DD19A_HUMAN | Q9NUU7 | | 5 | 1 | ATP-dependent RNA |
| | | | | | | helicase DDX19A |
| | DDX1_HUMAN | Q92499 | | 440 | 1 | ATP-dependent RNA |
| | | | | | | helicase DDX1 |
| 24 | DDX24_HUMAN | Q9GZR7 | ALPDDTVIESEAL | 297 | 1 | ATP-dependent RNA |
| | | | PSDIAAEAR | | | helicase DDX24 |
| | DDX46_HUMAN | Q7L014 | | 872 | 1 | Probable ATP-dependent |
| | | | | | | RNA helicase DDX46 |
| | DDX46_HUMAN | Q7L014 | | 923 | 1 | Probable ATP-dependent |
| | | | | | | RNA helicase DDX46 |
| | DDX46_HUMAN | Q7L014 | | 872 | 1 | Probable ATP-dependent |
| | | | | | | RNA helicase DDX46 |
| | DDX46_HUMAN | Q7L014 | | 872 | 1 | Probable ATP-dependent |
| | | | | | | RNA helicase DDX46 |
| 25 | DDX59_HUMAN | Q5T1V6 | AVATEAATIDR | 44 | 1 | Probable ATP-dependent |
| | | | | | | RNA helicase DDX59 |
| | DESM_HUMAN | P17661 | | 265 | 1 | Desmin |
| | DESM_HUMAN | P17661 | | 265 | 1 | Desmin |
| | DFFA_HUMAN | O00273 | | 7 | 1 | DNA fragmentation factor |
| | | | | | | subunit alpha |
| | DFFA_HUMAN | O00273 | | 222 | 1 | DNA fragmentation factor |
| | | | | | | subunit alpha |
| | DFFA_HUMAN | O00273 | | 222 | 1 | DNA fragmentation factor |
| | | | | | | subunit alpha |
| 26 | DGCR8_HUMAN | Q8WYQ5 | ALLEEGLCAPK | 249 | 1 | Protein DGCR8 |
| | DGCR8_HUMAN | Q8WYQ5 | | 397 | 1 | Protein DGCR8 |
| 27 | DGCR8_HUMAN | Q8WYQ5 | SMGADPGPPDEK | 397 | 1 | Protein DGCR8 |
| | | | DPLGAEAAPGAL | | | |
| | | | GQVK | | | |
| 28 | DGCR8_HUMAN | Q8WYQ5 | SMGADPGPPDEK | 397 | 1 | Protein DGCR8 |
| | | | DPLGAEAAPGAL | | | |
| | | | GQVKAK | | | |
| | DGKH_HUMAN | Q86XP1 | | 583 | 1 | Diacylglycerol kinase eta |
| 29 | DGKH_HUMAN | Q86XP1 | SVPGPAVAASKE | 699 | 1 | Diacylglycerol kinase eta |
| | | | NLPVLNTR | | | |
| | DGLB_HUMAN | Q8NCG7 | | 549 | 1 | Sn1-specific diacylglycerol |
| | | | | | | lipase beta |
| | DHAK_HUMAN | Q3LXA3 | | 363 | 1 | Dihydroxyacetone kinase |
| | DHAK_HUMAN | Q3LXA3 | | 363 | 1 | Dihydroxyacetone kinase |
| | DHX30_HUMAN | Q7L2E3 | | 207 | 1 | Putative ATP-dependent |
| | | | | | | RNA helicase DHX30 |
| | DHX37_HUMAN | Q8IY37 | | 574 | 1 | Probable ATP-dependent |
| | | | | | | RNA helicase DHX37 |
| | DHX9_HUMAN | Q08211 | | 168 | 1 | ATP-dependent RNA |
| | | | | | | helicase A |
| | DHX9_HUMAN | Q08211 | | 97 | 1 | ATP-dependent RNA |
| | | | | | | helicase A |
| | DHX9_HUMAN | Q08211 | | 97 | 1 | ATP-dependent RNA |
| | | | | | | helicase A |
| | DIAP1_HUMAN | O60610 | | 649 | 1 | Protein diaphanous homolog 1 |
| | DIDO1_HUMAN | Q9BTC0 | | 1251 | 1 | Death-inducer obliterator 1 |
| | DIDO1_HUMAN | Q9BTC0 | | 1519 | 1 | Death-inducer obliterator 1 |
| | DIDO1_HUMAN | Q9BTC0 | | 1353 | 1 | Death-inducer obliterator 1 |
| | DIDO1_HUMAN | Q9BTC0 | | 1353 | 1 | Death-inducer obliterator 1 |
| | DIDO1_HUMAN | Q9BTC0 | | 988 | 1 | Death-inducer obliterator 1 |
| | DLG1_HUMAN | Q12959 | | 413 | 1 | Disks large homolog 1 |
| | DNJC7_HUMAN | Q99615 | | 9 | 1 | DnaJ homolog subfamily C |
| | | | | | | member 7 |
| | DNJC7_HUMAN | Q99615 | | 9 | 1 | DnaJ homolog subfamily C |
| | | | | | | member 7 |
| 30 | DNJC7_HUMAN | Q99615 | VVMAATEPELLD | 9 | 1 | DnaJ homolog subfamily C |
| | | | DQEAK | | | member 7 |
| 31 | DNJC7_HUMAN | Q99615 | VVMAATEPELLD | 9 | 1 | DnaJ homolog subfamily C |
| | | | DQEAKR | | | member 7 |
| | DNM1L_HUMAN | O00429 | | 580 | 1 | Dynamin-1-like protein |
| | DNM1L_HUMAN | O00429 | | 504 | 1 | Dynamin-1-like protein |
| | DNM1L_HUMAN | O00429 | | 504 | 1 | Dynamin-1-like protein |
| | DNM1L_HUMAN | O00429 | | 504 | 1 | Dynamin-1-like protein |
| | DNM1L_HUMAN | O00429 | | 504 | 1 | Dynamin-1-like protein |
| 32 | DNM3A_HUMAN | Q9Y6K1 | MWVEPEAAAYAP | 439 | 1 | DNA (cytosine-5)- |
| | | | PPPAKKPR | | | methyltransferase 3A |
| | DOC10_HUMAN | Q96BY6 | | 328 | 1 | Dedicator of cytokinesis |
| | | | | | | protein 10 |
| 33 | DOHH_HUMAN | Q9BU89 | AIGQTLVDPK | 9 | 1 | Deoxyhypusine hydroxylase |
| 34 | DOHH_HUMAN | Q9BU89 | AIGQTLVDPKQPL | 9 | 1 | Deoxyhypusine hydroxylase |
| | | | QAR | | | |
| | DOT1L_HUMAN | Q8TEK3 | | 1334 | 1 | Histone-lysine N- |
| | | | | | | methyltransferase, H3 lysine- |
| | | | | | | 79 specific |
| 35 | DP13A_HUMAN | Q9UKG1 | SLVAPDTPIQFDII | 445 | 1 | DCC-interacting protein 13- |
| | | | SPVCEDQPGQAK | | | alpha |
| 36 | DPOD1_HUMAN | P28340 | HYVGPAQPVPGG | 103 | 1 | DNA polymerase delta |
| | | | PPPSR | | | catalytic subunit |
| 37 | DPOD1_HUMAN | P28340 | HYVGPAQPVPGG | 103 | 1 | DNA polymerase delta |
| | | | PPPSRGSVPVLR | | | catalytic subunit |
| 38 | DPOLA_HUMAN | P09884 | GIGYVEDGR | 84 | 1 | DNA polymerase alpha |
| | | | | | | catalytic subunit |
| | DPP9_HUMAN | Q86TI2 | | 14 | 1 | Dipeptidyl peptidase 9 |
| | DPYL4_HUMAN | O14531 | | 457 | 1 | Dihydropyrimidinase-related |
| | | | | | | protein 4 |
| | DREB_HUMAN | Q16643 | | 341 | 1 | Drebrin |
| | DREB_HUMAN | Q16643 | | 478 | 1 | Drebrin |
| | DSRAD_HUMAN | P55265 | | 215 | 1 | Double-stranded RNA- |
| | | | | | | specific adenosine deaminase |
| | DTL_HUMAN | Q9NZJ0 | | 579 | 1 | Denticleless protein homolog |
| | DTL_HUMAN | Q9NZJ0 | | 579 | 1 | Denticleless protein homolog |
| | DTX3L_HUMAN | Q8TDB6 | | 218 | 1 | Protein deltex-3-like |
| | DYHC1_HUMAN | Q14204 | | 4368 | 1 | Cytoplasmic dynein 1 heavy |
| | | | | | | chain 1 |
| | DYHC1_HUMAN | Q14204 | | 4221 | 1 | Cytoplasmic dynein 1 heavy |
| | | | | | | chain 1 |
| | E400N_HUMAN | Q6ZTU2 | | 184 | 2 | EP400 N-terminal-like |
| | | | | | | protein |
| | EP400_HUMAN | Q96L91 | | 195 | | E1A-binding protein p400 |
| | E41L2_HUMAN | O43491 | | 913 | 1 | Band 4.1-like protein 2 |
| | EAP1_HUMAN | Q9H1B7 | | 133 | 1 | Enhanced at puberty protein 1 |
| | EBP2_HUMAN | Q99848 | | 212 | 1 | Probable rRNA-processing |
| | | | | | | protein EBP2 |
| | ECE1_HUMAN | P42892 | | 34 | 1 | Endothelin-converting |
| | | | | | | enzyme 1 |
| 39 | ECT2_HUMAN | Q9H8V3 | GCPANLLSSHR | 629 | 1 | Protein ECT2 |
| | EDC4_HUMAN | Q6P2E9 | | 797 | 1 | Enhancer of mRNA- |
| | | | | | | decapping protein 4 |
| | EDC4_HUMAN | Q6P2E9 | | 663 | 1 | Enhancer of mRNA- |
| | | | | | | decapping protein 4 |
| | EDC4_HUMAN | Q6P2E9 | | 663 | 1 | Enhancer of mRNA- |
| | | | | | | decapping protein 4 |
| | EDC4_HUMAN | Q6P2E9 | | 491 | 1 | Enhancer of mRNA- |
| | | | | | | decapping protein 4 |
| | EDC4_HUMAN | Q6P2E9 | | 486 | 1 | Enhancer of mRNA- |
| | | | | | | decapping protein 4 |
| 40 | EDC4_HUMAN | Q6P2E9 | SLGADGTHGAGA | 486 | 1 | Enhancer of mRNA- |
| | | | MESAAGVLIK | | | decapping protein 4 |
| | EDC4_HUMAN | Q6P2E9 | | 58 | 1 | Enhancer of mRNA- |
| | | | | | | decapping protein 4 |
| 41 | EDRF1_HUMAN | Q3B7T1 | SVGNDVDVVSDS | 116 | 1 | Erythroid differentiation- |
| | | | ENIK | | | related factor 1 |
| 42 | EDRF1_HUMAN | Q3B7T1 | SVGNDVDVVSDS | 116 | 1 | Erythroid differentiation- |
| | | | ENIKK | | | related factor 1 |
| 43 | EEA1_HUMAN | Q15075 | SSAELQSLEQQLE | 133 | 1 | Early endosome antigen 1 |
| | | | EAQTENFNIK | | | |
| 44 | EEA1_HUMAN | Q15075 | GLVTDSSAELQSL | 128 | 1 | Early endosome antigen 1 |
| | | | EQQLEEAQTENF | | | |
| | | | NIK | | | |
| | EF1A1_HUMAN | P68104 | | 399 | 2 | Elongation factor 1-alpha 1 |
| | EF1A3_HUMAN | Q5VTE0 | | 399 | | Putative elongation factor 1- |
| | | | | | | alpha-like 3 |
| | EF1A1_HUMAN | P68104 | | 404 | 2 | Elongation factor 1-alpha 1 |
| | EF1A3_HUMAN | Q5VTE0 | | 404 | | Putative elongation factor 1- |
| | | | | | | alpha-like 3 |
| | EF1A1_HUMAN | P68104 | | 404 | 2 | Elongation factor 1-alpha 1 |
| | EF1A3_HUMAN | Q5VTE0 | | 404 | | Putative elongation factor 1- |
| | | | | | | alpha-like 3 |
| | EF1A1_HUMAN | P68104 | | 200 | 2 | Elongation factor 1-alpha 1 |
| | EF1A3_HUMAN | Q5VTE0 | | 200 | | Putative elongation factor 1- |
| | | | | | | alpha-like 3 |
| | EF1A1_HUMAN | P68104 | | 234 | 2 | Elongation factor 1-alpha 1 |
| | EF1A3_HUMAN | Q5VTE0 | | 234 | | Putative elongation factor 1- |
| | | | | | | alpha-like 3 |
| | EF1A1_HUMAN | P68104 | | 404 | 2 | Elongation factor 1-alpha 1 |
| | EF1A3_HUMAN | Q5VTE0 | | 404 | | Putative elongation factor 1- |
| | | | | | | alpha-like 3 |
| 45 | EF1B_HUMAN | P24534 | LFGSDDEEESEEA | 103 | 1 | Elongation factor 1-beta |
| | | | KR | | | |
| 46 | EF1B_HUMAN | P24534 | LFGSDDEEESEEAK | 103 | 1 | Elongation factor 1-beta |
| | EF1D_HUMAN | P29692 | | 159 | 1 | Elongation factor 1-delta |
| | EF1D_HUMAN | P29692 | | 159 | 1 | Elongation factor 1-delta |
| | EF2_HUMAN | P13639 | | 612 | 1 | Elongation factor 2 |
| 47 | EH1L1_HUMAN | Q8N3D4 | SQQPPGGSSPSEE | 1330 | 1 | EH domain-binding protein |
| | | | PPPSPGEEAGLQR | | | 1-like protein 1 |
| | EHBP1_HUMAN | Q8NDI1 | | 275 | 1 | EH domain-binding protein 1 |
| | EHD1_HUMAN | Q9H4M9 | | 416 | 1 | EH domain-containing |
| | | | | | | protein 1 |
| | EHMT1_HUMAN | Q9H9B1 | | 330 | 1 | Histone-lysine N- |
| | | | | | | methyltransferase, H3 lysine- |
| | | | | | | 9 specific 5 |
| | EHMT1_HUMAN | Q9H9B1 | | 482 | 1 | Histone-lysine N- |
| | | | | | | methyltransferase, H3 lysine- |
| | | | | | | 9 specific 5 |
| | EHMT2_HUMAN | Q96KQ7 | | 454 | 1 | Histone-lysine N- |
| | | | | | | methyltransferase, H3 lysine- |
| | | | | | | 9 specific 3 |
| | EIF3B_HUMAN | P55884 | | 4 | 1 | Eukaryotic translation |
| | | | | | | initiation factor 3 subunit B |
| | EIF3B_HUMAN | P55884 | | 185 | 1 | Eukaryotic translation |
| | | | | | | initiation factor 3 subunit B |
| | EIF3G_HUMAN | O75821 | | 8 | 1 | Eukaryotic translation |
| | | | | | | initiation factor 3 subunit G |
| 48 | EIF3J_HUMAN | O75822 | NWDDDDDEKKE | 51 | 1 | Eukaryotic translation |
| | | | EAEVKPEVK | | | initiation factor 3 subunit J |
| | ELF1_HUMAN | P32519 | | 146 | 1 | ETS-related transcription |
| | | | | | | factor Elf-1 |
| 49 | ELF1_HUMAN | P32519 | GIPEVMETQQVQ | 146 | 1 | ETS-related transcription |
| | | | EK | | | factor Elf-1 |
| | ENOA_HUMAN | P06733 | | 204 | 1 | Alpha-enolase |
| | ENPL_HUMAN | P14625 | | 60 | 1 | Endoplasmin |
| | ENPL_HUMAN | P14625 | | 29 | 1 | Endoplasmin |
| | ENPL_HUMAN | P14625 | | 29 | 1 | Endoplasmin |
| | EP15R_HUMAN | Q9UBC2 | | 570 | 1 | Epidermal growth factor |
| | | | | | | receptor substrate 15-like 1 |
| | EP15_HUMAN | P42566 | | 619 | 1 | Epidermal growth factor |
| | | | | | | receptor substrate 15 |
| | EPC1_HUMAN | Q9H2F5 | | 28 | 1 | Enhancer of polycomb |
| | | | | | | homolog 1 |
| | EPN1_HUMAN | Q9Y6I3 | | 461 | 1 | Epsin-1 |
| | EPN2_HUMAN | O95208 | | 340 | 1 | Epsin-2 |
| | ERC6L_HUMAN | Q2NKX8 | | 802 | 1 | DNA excision repair protein |
| | | | | | | ERCC-6-like |
| | ERCC6_HUMAN | Q03468 | | 53 | 1 | DNA excision repair protein |
| | | | | | | ERCC-6 |
| | ERF3A_HUMAN | P15170 | | 40 | 1 | Eukaryotic peptide chain |
| | | | | | | release factor GTP-binding |
| | | | | | | subunit ERF3A |
| | ERF3A_HUMAN | P15170 | | 40 | 1 | Eukaryotic peptide chain |
| | | | | | | release factor GTP-binding |
| | | | | | | subunit ERF3A |
| | ERF3A_HUMAN | P15170 | | 40 | 1 | Eukaryotic peptide chain |
| | | | | | | release factor GTP-binding |
| | | | | | | subunit ERF3A |
| 50 | ERF3A_HUMAN | P15170 | GRPPEESAHEMM | 40 | 1 | Eukaryotic peptide chain |
| | | | EEEEEIPKPK | | | release factor GTP-binding |
| | | | | | | subunit ERF3A |
| 51 | ERF_HUMAN | P50548 | GTSELEEPLGEDPR | 192 | 1 | ETS domain-containing |
| | | | | | | transcription factor ERF |
| | ERIC1_HUMAN | Q86X53 | | 277 | 1 | Glutamate-rich protein 1 |
| | ESYT2_HUMAN | A0FGR8 | | 760 | 1 | Extended synaptotagmin-2 |
| | ETUD1_HUMAN | Q7Z2Z2 | | 933 | 1 | Elongation factor Tu GTP- |
| | | | | | | binding domain-containing |
| | | | | | | protein 1 |
| | ETUD1_HUMAN | Q7Z2Z2 | | 933 | 1 | Elongation factor Tu GTP- |
| | | | | | | binding domain-containing |
| | | | | | | protein 1 |
| | EXDL2_HUMAN | Q9NVH0 | | 199 | 1 | Exonuclease 3′-5′ domain- |
| | | | | | | like-containing protein 2 |
| 52 | F101B_HUMAN | Q8N5W9 | AAAATPAAPSPAS | 62 | 1 | Protein FAM101B |
| | | | LPLAPGCALR | | | |
| | F107B_HUMAN | Q9H098 | | 6 | 1 | Protein FAM107B |
| 53 | F117B_HUMAN | Q6P1L5 | GHRAPPPLVQR | 375 | 1 | Protein FAM117B |
| 54 | F125A_HUMAN | Q96EY5 | AASQPSKGGLLER | 173 | 1 | Protein FAM125A |
| | F169A_HUMAN | Q9Y6X4 | | 447 | 1 | UPF0611 protein FAM169A |
| | FA13A_HUMAN | O94988 | | 595 | 1 | Protein FAM13A1 |
| | FA13A_HUMAN | O94988 | | 595 | 1 | Protein FAM13A1 |
| | FA21A_HUMAN | Q641Q2 | | 1135 | 4 | Protein FAM21A |
| | FA21B_HUMAN | Q5SNT6 | | 1047 | | Protein FAM21B |
| | FA21C_HUMAN | Q9Y4E1 | | 1114 | | Protein FAM21C |
| | FA21D_HUMAN | Q5SRD0 | | 102 | | Protein FAM21D |
| | FA29A_HUMAN | Q7Z4H7 | | 569 | 1 | Protein FAM29A |
| 55 | FA44A_HUMAN | Q8NFC6 | GLMATTASGDIT | 2045 | 1 | Protein FAM44A |
| | | | NQNSLAGGKNQGK | | | |
| | FA44A_HUMAN | Q8NFC6 | | 1484 | 1 | Protein FAM44A |
| | FA44A_HUMAN | Q8NFC6 | | 2045 | 1 | Protein FAM44A |
| | FA44A_HUMAN | Q8NFC6 | | 2045 | 1 | Protein FAM44A |
| | FA44A_HUMAN | Q8NFC6 | | 2045 | 1 | Protein FAM44A |
| | FA44A_HUMAN | Q8NFC6 | | 1709 | 1 | Protein FAM44A |
| | FAS_HUMAN | P49327 | | 1166 | 1 | Fatty acid synthase |
| | FETUA_HUMAN | P02765 | | 134 | 1 | Alpha-2-HS-glycoprotein |
| | FIP1_HUMAN | Q6UN15 | | 159 | 1 | Pre-mRNA 3′-end-processing |
| | | | | | | factor FIP1 |
| | FKB15_HUMAN | Q5T1M5 | | 307 | 1 | FK506-binding protein 15 |
| | FKB15_HUMAN | Q5T1M5 | | 307 | 1 | FK506-binding protein 15 |
| | FLI1_HUMAN | Q01543 | | 21 | 1 | Friend leukemia integration 1 |
| | | | | | | transcription factor |
| 56 | FLNA_HUMAN | P21333 | GSPVPSSPFQVPV | 1337 | 1 | Filamin-A |
| | | | TEGCDPSR | | | |
| 57 | FLNA_HUMAN | P21333 | GSPVPSSPFQVPV | 1337 | 1 | Filamin-A |
| | | | TEGCDPSRVR | | | |
| | FLNA_HUMAN | P21333 | | 2537 | 1 | Filamin-A |
| | FLNA_HUMAN | P21333 | | 26 | 1 | Filamin-A |
| | FLNA_HUMAN | P21333 | | 1505 | 1 | Filamin-A |
| 58 | FLNA_HUMAN | P21333 | GVPVPGSPFPLEA | 1049 | 1 | Filamin-A |
| | | | VAPTKPSK | | | |
| 59 | FLNA_HUMAN | P21333 | GVPVPGSPFPLEA | 1049 | 1 | Filamin-A |
| | | | VAPTKPSKVK | | | |
| 60 | FLNA_HUMAN | P21333 | GVPVPGSPFPLEA | 1049 | 1 | Filamin-A |
| | | | VAPTKPSKVKAF | | | |
| | | | GPGLQGGSAGSP | | | |
| | | | AR | | | |
| | FLNA_HUMAN | P21333 | | 35 | 3 | Filamin-A |
| | FLNB_HUMAN | O75369 | | 8 | | Filamin-B |
| | FLNC_HUMAN | Q14315 | | 28 | | Filamin-C |
| | FLNB_HUMAN | O75369 | | 479 | 1 | Filamin-B |
| | FLNB_HUMAN | O75369 | | 1022 | 1 | Filamin-B |
| | FLNB_HUMAN | O75369 | | 1477 | 1 | Filamin-B |
| | FLNB_HUMAN | O75369 | | 1477 | 1 | Filamin-B |
| | FNBP1_HUMAN | Q96RU3 | | 520 | 1 | Formin-binding protein 1 |
| | FNBP1_HUMAN | Q96RU3 | | 520 | 1 | Formin-binding protein 1 |
| | FNBP4_HUMAN | Q8N3X1 | | 154 | 1 | Formin-binding protein 4 |
| | FNBP4_HUMAN | Q8N3X1 | | 426 | 1 | Formin-binding protein 4 |
| | FNBP4_HUMAN | Q8N3X1 | | 778 | 1 | Formin-binding protein 4 |
| | FOXJ2_HUMAN | Q9P0K8 | | 213 | 1 | Forkhead box protein J2 |
| 61 | FOXK1_HUMAN | P85037 | SAVAGAAPALVA | 81 | 1 | Forkhead box protein K1 |
| | | | AAAASVR | | | |
| | FOXO3_HUMAN | O43524 | | 55 | 1 | Forkhead box protein O3 |
| 62 | FOXP4_HUMAN | Q8IVH2 | GLVHPPTSAAAPV | 407 | 1 | Forkhead box protein P4 |
| | | | TPLRPPGLGSASL | | | |
| | | | HGGGPAR | | | |
| | FRAP_HUMAN | P42345 | | 2460 | 1 | FKBP12-rapamycin |
| | | | | | | complex-associated protein |
| | FRAP_HUMAN | P42345 | | 2460 | 1 | FKBP12-rapamycin |
| | | | | | | complex-associated protein |
| | FRYL_HUMAN | O94915 | | 1513 | 1 | Protein furry homolog-like |
| | FUBP1_HUMAN | Q96AE4 | | 182 | 1 | Far upstream element- |
| | | | | | | binding protein 1 |
| | FUBP1_HUMAN | Q96AE4 | | 84 | 1 | Far upstream element- |
| | | | | | | binding protein 1 |
| | FUBP1_HUMAN | Q96AE4 | | 140 | 2 | Far upstream element- |
| | | | | | | binding protein 1 |
| | FUBP2_HUMAN | Q92945 | | 184 | | Far upstream element- |
| | | | | | | binding protein 2 |
| 63 | FUBP2_HUMAN | Q92945 | SISSQLGPIHPPPR | 129 | 1 | Far upstream element- |
| | | | | | | binding protein 2 |
| 64 | FUBP3_HUMAN | Q96I24 | SNSTIQEILIPASK | 160 | 1 | Far upstream element- |
| | | | | | | binding protein 3 |
| | FUBP3_HUMAN | Q96I24 | | 35 | 1 | Far upstream element- |
| | | | | | | binding protein 3 |
| 65 | FUS_HUMAN | P35637 | GKEFSGNPIKVSF | 356 | 1 | RNA-binding protein FUS |
| | | | ATR | | | |
| 66 | FUS_HUMAN | P35637 | GKEFSGNPIK | 356 | 1 | RNA-binding protein FUS |
| | FXR2_HUMAN | P51116 | | 562 | 1 | Fragile X mental retardation |
| | | | | | | syndrome-related protein 2 |
| | FYB_HUMAN | O15117 | | 656 | 1 | FYN-binding protein |
| 67 | FYB_HUMAN | O15117 | GAGNLDEEQDSE | 447 | 1 | FYN-binding protein |
| | | | GETYEDIEASK | | | |
| 68 | FYB_HUMAN | O15117 | GAGNLDEEQDSE | 447 | 1 | FYN-binding protein |
| | | | GETYEDIEASKER | | | |
| | FYN_HUMAN | P06241 | | 20 | 1 | Proto-oncogene tyrosine- |
| | | | | | | protein kinase Fyn |
| | FYTD1_HUMAN | Q96QD9 | | 327 | 2 | Forty-two-three domain- |
| | | | | | | containing protein 1 |
| | THOC4_HUMAN | Q86V81 | | | | THO complex subunit 4 |
| | FYV1_HUMAN | Q9Y2I7 | | 1608 | 1 | FYVE finger-containing |
| | | | | | | phosphoinositide kinase |
| | FYV1_HUMAN | Q9Y2I7 | | 1608 | 1 | FYVE finger-containing |
| | | | | | | phosphoinositide kinase |
| | FYV1_HUMAN | Q9Y2I7 | | 990 | 1 | FYVE finger-containing |
| | | | | | | phosphoinositide kinase |
| | G3P_HUMAN | P04406 | | 90 | 1 | Glyceraldehyde-3-phosphate |
| | | | | | | dehydrogenase |
| | GABP1_HUMAN | Q06547 | | 304 | 1 | GA-binding protein subunit |
| | | | | | | beta-1 |
| | GABP1_HUMAN | Q06547 | | 304 | 1 | GA-binding protein subunit |
| | | | | | | beta-1 |
| | GABP2_HUMAN | Q8TAK5 | | 305 | 1 | GA-binding protein subunit |
| | | | | | | beta-2 |
| | GALT_HUMAN | P07902 | | 19 | 1 | Galactose-1-phosphate |
| | | | | | | uridylyltransferase |
| 69 | GAPD1_HUMAN | Q14C86 | SASQAAHPQDSA | 1103 | 1 | GTPase-activating protein |
| | | | FSYR | | | and VPS9 domain-containing |
| | | | | | | protein 1 |
| 70 | GAPD1_HUMAN | Q14C86 | SASQAAHPQDSA | 1103 | 1 | GTPase-activating protein |
| | | | FSYRDAK | | | and VPS9 domain-containing |
| | | | | | | protein 1 |
| | GATA2_HUMAN | P23769 | | 47 | 1 | Endothelial transcription |
| | | | | | | factor GATA-2 |
| 71 | GBF1_HUMAN | Q92538 | SASVHDMDYVNPR | 369 | 1 | Golgi-specific brefeldin A- |
| | | | | | | resistance guanine nucleotide |
| | | | | | | exchange factor 1 |
| | GBF1_HUMAN | Q92538 | SASVHDMDYVNPR | 369 | 1 | Golgi-specific brefeldin A- |
| | | | | | | resistance guanine nucleotide |
| | | | | | | exchange factor 1 |
| | GCFC_HUMAN | Q9Y5B6 | | 222 | 1 | GC-rich sequence DNA- |
| | | | | | | binding factor homolog |
| | GCP2_HUMAN | Q9BSJ2 | | 773 | 1 | Gamma-tubulin complex |
| | | | | | | component 2 |
| | GCP60_HUMAN | Q9H3P7 | | 16 | 1 | Golgi resident protein |
| | | | | | | GCP60 |
| 72 | GCP60_HUMAN | Q9H3P7 | SSEKELEPEAAEE | 344 | 1 | Golgi resident protein |
| | | | ALENGPK | | | GCP60 |
| | GDIR2_HUMAN | P52566 | | 20 | 1 | Rho GDP-dissociation |
| | | | | | | inhibitor 2 |
| | GDIR2_HUMAN | P52566 | | 20 | 1 | Rho GDP-dissociation |
| | | | | | | inhibitor 2 |
| | GDIR2_HUMAN | P52566 | | 56 | 1 | Rho GDP-dissociation |
| | | | | | | inhibitor 2 |
| | GELS_HUMAN | P06396 | | 640 | 1 | Gelsolin |
| 73 | GELS_HUMAN | P06396 | GLGLSYLSSHIAN | 404 | 1 | Gelsolin |
| | | | VER | | | |
| 74 | GEMI5_HUMAN | Q8TEQ6 | TASTEETDPETSQ | 1320 | 1 | Gem-associated protein 5 |
| | | | PEPNRPSELDLR | | | |
| | GEMI8_HUMAN | Q9NWZ8 | | 170 | 1 | Gem-associated protein 8 |
| | GEN_HUMAN | Q17RS7 | | 624 | 1 | Flap endonuclease GEN |
| | | | | | | homolog 1 |
| | GFPT1_HUMAN | Q06210 | | 261 | 1 | Glucosamine--fructose-6- |
| | | | | | | phosphate aminotransferase |
| | | | | | | [isomerizing] 1 |
| | GGA3_HUMAN | Q9NZ52 | | 334 | 1 | ADP-ribosylation factor- |
| | | | | | | binding protein GGA3 |
| | GGA3_HUMAN | Q9NZ52 | | 518 | 1 | ADP-ribosylation factor- |
| | | | | | | binding protein GGA3 |
| | GIT1_HUMAN | Q9Y2X7 | | 633 | 1 | ARF GTPase-activating |
| | | | | | | protein GIT1 |
| | GIT1_HUMAN | Q9Y2X7 | | 419 | 1 | ARF GTPase-activating |
| | | | | | | protein GIT1 |
| | GIT1_HUMAN | Q9Y2X7 | | 419 | 1 | ARF GTPase-activating |
| | | | | | | protein GIT1 |
| | GIT1_HUMAN | Q9Y2X7 | | 633 | 1 | ARF GTPase-activating |
| | | | | | | protein GIT1 |
| | GIT2_HUMAN | Q14161 | | 626 | 1 | ARF GTPase-activating |
| | | | | | | protein GIT2 |
| | GLGB_HUMAN | Q04446 | | 308 | 1 | 1,4-alpha-glucan-branching |
| | | | | | | enzyme |
| | GLRX3_HUMAN | O76003 | | 102 | 1 | Glutaredoxin-3 |
| | GLRX3_HUMAN | O76003 | | 102 | 1 | Glutaredoxin-3 |
| | GLU2B_HUMAN | P14314 | | 102 | 1 | Glucosidase 2 subunit beta |
| | GLU2B_HUMAN | P14314 | | 102 | 1 | Glucosidase 2 subunit beta |
| | GLU2B_HUMAN | P14314 | | 227 | 1 | Glucosidase 2 subunit beta |
| | GLU2B_HUMAN | P14314 | | 95 | 1 | Glucosidase 2 subunit beta |
| 75 | GMIP_HUMAN | Q9P107 | GGGEVSSQGPEDS | 843 | 1 | GEM-interacting protein |
| | | | LLGTQSR | | | |
| | GMIP_HUMAN | Q9P107 | | 425 | 1 | GEM-interacting protein |
| | GMIP_HUMAN | Q9P107 | | 473 | 1 | GEM-interacting protein |
| 76 | GNL1_HUMAN | P36915 | SAMEPTGPTQER | 344 | 1 | Guanine nucleotide-binding |
| | | | | | | protein-like 1 |
| 77 | GNL1_HUMAN | P36915 | SAMEPTGPTQER | 344 | 1 | Guanine nucleotide-binding |
| | | | YKDGVVTIGCVG | | | protein-like 1 |
| | | | FPNVGK | | | |
| | GNL1_HUMAN | P36915 | | 53 | 1 | Guanine nucleotide-binding |
| | | | | | | protein-like 1 |
| | GNL1_HUMAN | P36915 | | 344 | 1 | Guanine nucleotide-binding |
| | | | | | | protein-like 1 |
| | GNL1_HUMAN | P36915 | | 50 | 1 | Guanine nucleotide-binding |
| | | | | | | protein-like 1 |
| | GOGB1_HUMAN | Q14789 | | 1246 | 1 | Golgin subfamily B member 1 |
| | GOGB1_HUMAN | Q14789 | | 1802 | 1 | Golgin subfamily B member 1 |
| 78 | GOGB1_HUMAN | Q14789 | SLSMSTRPTCSES | 1802 | 1 | Golgin subfamily B member 1 |
| | | | VPSAK | | | |
| | GON4L_HUMAN | Q3T8J9 | | 482 | 1 | GON-4-like protein |
| 79 | GPKOW_HUMAN | Q92917 | GAGPSPEEKDFLK | 38 | 1 | G patch domain and KOW |
| | | | | | | motifs-containing protein |
| 80 | GPKOW_HUMAN | Q92917 | GAGPSPEEK | 38 | 1 | G patch domain and KOW |
| | | | | | | motifs-containing protein |
| 81 | GPKOW_HUMAN | Q92917 | GAGPSPEEKDFLK | 38 | 1 | G patch domain and KOW |
| | | | TVEGR | | | motifs-containing protein |
| | GPKOW_HUMAN | Q92917 | | 99 | 1 | G patch domain and KOW |
| | | | | | | motifs-containing protein |
| | GPKOW_HUMAN | Q92917 | | 99 | 1 | G patch domain and KOW |
| | | | | | | motifs-containing protein |
| | GPN1_HUMAN | Q9HCN4 | | 312 | 1 | GPN-loop GTPase 1 |
| | GPTC8_HUMAN | Q9UKJ3 | | 883 | 1 | G patch domain-containing |
| | | | | | | protein 8 |
| | GRDN_HUMAN | Q3V6T2 | | 220 | 1 | Girdin |
| | GRDN_HUMAN | Q3V6T2 | | 485 | 1 | Girdin |
| | GRIN1_HUMAN | Q7Z2K8 | | 307 | 1 | G protein-regulated inducer |
| | | | | | | of neurite outgrowth 1 |
| 82 | GSDMD_HUMAN | P57764 | GQIQGSVELAAPG | 88 | 1 | Gasdermin-D |
| | | | QAK | | | |
| 83 | GSDMD_HUMAN | P57764 | GVPAEGAFTEDF | 276 | 1 | Gasdermin-D |
| | | | QGLR | | | |
| | GSTP1_HUMAN | P09211 | | 92 | 1 | Glutathione S-transferase P |
| | GSTP1_HUMAN | P09211 | | 92 | 1 | Glutathione S-transferase P |
| | GTF2I_HUMAN | P78347 | | 106 | 1 | General transcription factor |
| | | | | | | II-I |
| | GTF2I_HUMAN | P78347 | | 106 | 1 | General transcription factor |
| | | | | | | II-I |
| | H2AY_HUMAN | O75367 | | 173 | 1 | Core histone macro-H2A.1 |
| | H4_HUMAN | P62805 | | 70 | 1 | Histone H4 |
| | H4_HUMAN | P62805 | | 70 | 1 | Histone H4 |
| | H4_HUMAN | P62805 | | 70 | 1 | Histone H4 |
| 84 | H4_HUMAN | P62805 | NIQGITKPAIR | 26 | 1 | Histone H4 |
| | HAP28_HUMAN | Q13442 | | 25 | 1 | 28 kDa heat- and acid-stable |
| | | | | | | phosphoprotein |
| | HAP28_HUMAN | Q13442 | | 25 | 1 | 28 kDa heat- and acid-stable |
| | | | | | | phosphoprotein |
| | HBS1L_HUMAN | Q9Y450 | | 30 | 1 | HBS1-like protein |
| 85 | HCLS1_HUMAN | P14317 | FVNDISEKEQR | 27 | 1 | Hematopoietic lineage cell- |
| | | | | | | specific protein |
| 86 | HCLS1_HUMAN | P14317 | FVNDISEK | 27 | 1 | Hematopoietic lineage cell- |
| | | | | | | specific protein |
| | HDAC4_HUMAN | P56524 | | 9 | 1 | Histone deacetylase 4 |
| | HDAC4_HUMAN | P56524 | | 290 | 1 | Histone deacetylase 4 |
| | HDAC6_HUMAN | Q9UBN7 | | 1089 | 1 | Histone deacetylase 6 |
| | HDAC6_HUMAN | Q9UBN7 | | 1089 | 1 | Histone deacetylase 6 |
| 87 | HDAC7_HUMAN | Q8WUI4 | GGGPGQVVDDGL | 413 | 1 | Histone deacetylase 7 |
| | | | EHR | | | |
| | HDC_HUMAN | Q9UBI9 | | 324 | 1 | Headcase protein homolog |
| | HDGR2_HUMAN | Q7Z4V5 | | 31 | 1 | Hepatoma-derived growth |
| | | | | | | factor-related protein 2 |
| | HDGR2_HUMAN | Q7Z4V5 | | 242 | 1 | Hepatoma-derived growth |
| | | | | | | factor-related protein 2 |
| | HDGR2_HUMAN | Q7Z4V5 | | 242 | 1 | Hepatoma-derived growth |
| | | | | | | factor-related protein 2 |
| | HDGR2_HUMAN | Q7Z4V5 | | 31 | 1 | Hepatoma-derived growth |
| | | | | | | factor-related protein 2 |
| | HECD1_HUMAN | Q9ULT8 | | 1493 | 1 | E3 ubiquitin-protein ligase |
| | | | | | | HECTD1 |
| 88 | HELLS_HUMAN | Q9NRZ9 | TAVITPAMLEEEE | 23 | 1 | Lymphoid-specific helicase |
| | | | QLEAAGLER | | | |
| | HG2A_HUMAN | P04233 | | 23 | 1 | HLA class II |
| | | | | | | histocompatibility antigen |
| | | | | | | gamma chain |
| 89 | HG2A_HUMAN | P04233 | LISNNEQLPMLGR | 23 | 1 | HLA class II |
| | | | | | | histocompatibility antigen |
| | | | | | | gamma chain |
| | HIRP3_HUMAN | Q9BW71 | | 111 | 1 | HIRA-interacting protein 3 |
| 90 | HJURP_HUMAN | Q8NCD3 | GSVQAAAWGPEL | 92 | 1 | Holliday junction recognition |
| | | | PSHR | | | protein |
| 91 | HMHA1_HUMAN | Q92619 | GGAGASAFEQAD | 663 | 1 | Minor histocompatibility |
| | | | LNGMTPELPVAV | | | protein HA-1 |
| | | | PSGPFRHEGLSK | | | |
| 92 | HMHA1_HUMAN | Q92619 | AGCLPAEEVDVL | 263 | 1 | Minor histocompatibility |
| | | | LQR | | | protein HA-1 |
| 93 | HMHA1_HUMAN | Q92619 | AVFPGPSLEPPAG | 40 | 1 | Minor histocompatibility |
| | | | SSGVK | | | protein HA-1 |
| | HMOX2_HUMAN | P30519 | | 252 | 1 | Heme oxygenase 2 |
| | HMOX2_HUMAN | P30519 | | 252 | 1 | Heme oxygenase 2 |
| | HMOX2_HUMAN | P30519 | | 252 | 1 | Heme oxygenase 2 |
| | HNRH1_HUMAN | P31943 | | 341 | 1 | Heterogeneous nuclear |
| | | | | | | ribonucleoprotein H |
| | HNRH1_HUMAN | P31943 | | 95 | 2 | Heterogeneous nuclear |
| | | | | | | ribonucleoprotein H |
| | HNRH2_HUMAN | P55795 | | 95 | | Heterogeneous nuclear |
| | | | | | | ribonucleoprotein H2 |
| | HNRH1_HUMAN | P31943 | | 95 | 2 | Heterogeneous nuclear |
| | | | | | | ribonucleoprotein H |
| | HNRH2_HUMAN | P55795 | | 95 | | Heterogeneous nuclear |
| | | | | | | ribonucleoprotein H2 |
| | HNRH1_HUMAN | P31943 | | 252 | 2 | Heterogeneous nuclear |
| | | | | | | ribonucleoprotein H |
| | HNRH2_HUMAN | P55795 | | 252 | | Heterogeneous nuclear |
| | | | | | | ribonucleoprotein H2 |
| | HNRH2_HUMAN | P55795 | | 341 | 1 | Heterogeneous nuclear |
| | | | | | | ribonucleoprotein H2 |
| 94 | HNRH3_HUMAN | P31942 | GGYGGFDDYGGY | 145 | 1 | Heterogeneous nuclear |
| | | | NNYGYGNDGFDDR | | | ribonucleoprotein H3 |
| | HNRL1_HUMAN | Q9BUJ2 | | 97 | 1 | Heterogeneous nuclear |
| | | | | | | ribonucleoprotein U-like |
| | | | | | | protein 1 |
| 95 | HNRL1_HUMAN | Q9BUJ2 | GHYAMDNITR | 97 | 1 | Heterogeneous nuclear |
| | | | | | | ribonucleoprotein U-like |
| | | | | | | protein 1 |
| 96 | HNRL2_HUMAN | Q1KMD3 | ASEKPAEATAGS | 127 | 1 | Heterogeneous nuclear |
| | | | GGVNGGEEQGLGK | | | ribonucleoprotein U-like |
| | | | | | | protein 2 |
| 97 | HNRL2_HUMAN | Q1KMD3 | ASEKPAEATAGS | 127 | 1 | Heterogeneous nuclear |
| | | | GGVNGGEEQGLG | | | ribonucleoprotein U-like |
| | | | KR | | | protein 2 |
| | HNRLL_HUMAN | Q8WVV9 | | 290 | 1 | Heterogeneous nuclear |
| | | | | | | ribonucleoprotein L-like |
| | HNRPD_HUMAN | Q14103 | | 70 | 1 | Heterogeneous nuclear |
| | | | | | | ribonucleoprotein D0 |
| | HNRPF_HUMAN | P52597 | | 252 | 1 | Heterogeneous nuclear |
| | | | | | | ribonucleoprotein F |
| | HNRPG_HUMAN | P38159 | | 284 | 1 | Heterogeneous nuclear |
| | | | | | | ribonucleoprotein G |
| | HNRPG_HUMAN | P38159 | | 234 | 1 | Heterogeneous nuclear |
| | | | | | | ribonucleoprotein G |
| 98 | HNRPK_HUMAN | P61978 | AVECLNYQHYK | 129 | 1 | Heterogeneous nuclear |
| | | | | | | ribonucleoprotein K |
| 99 | HNRPK_HUMAN | P61978 | AVECLNYQHYKG | 129 | 1 | Heterogeneous nuclear |
| | | | SDFDCELR | | | ribonucleoprotein K |
| 100 | HNRPK_HUMAN | P61978 | SAIDTWSPSEWQ | 347 | 1 | Heterogeneous nuclear |
| | | | MAYEPQGGSGYD | | | ribonucleoprotein K |
| | | | YSYAGGR | | | |
| 101 | HNRPK_HUMAN | P61978 | YSYAGGR | 371 | 2 | Heterogeneous nuclear |
| | | | | | | ribonucleoprotein K |
| 102 | HNRPL_HUMAN | P14866 | YTNPNLSGQGDP | 285 | 1 | Heterogeneous nuclear |
| | | | GSNPNKR | | | ribonucleoprotein L |
| | HNRPQ_HUMAN | O60506 | | 469 | 1 | Heterogeneous nuclear |
| | | | | | | ribonucleoprotein Q |
| | HOOK1_HUMAN | Q9UJC3 | | 234 | 1 | Protein Hook homolog 1 |
| | HOOK1_HUMAN | Q9UJC3 | | 234 | 1 | Protein Hook homolog 1 |
| 103 | HOOK2_HUMAN | Q96ED9 | SLSPETYGNFDSQ | 161 | 1 | Protein Hook homolog 2 |
| | | | SR | | | |
| | HPS4_HUMAN | Q9NQG7 | | 496 | 1 | Hermansky-Pudlak |
| | | | | | | syndrome 4 protein |
| | HRX_HUMAN | Q03164 | | 2719 | 1 | Histone-lysine N- |
| | | | | | | methyltransferase HRX |
| | HRX_HUMAN | Q03164 | | 2719 | 1 | Histone-lysine N- |
| | | | | | | methyltransferase HRX |
| | HRX_HUMAN | Q03164 | | 2385 | 1 | Histone-lysine N- |
| | | | | | | methyltransferase HRX |
| | HS105_HUMAN | Q92598 | | 548 | 1 | Heat shock protein 105 kDa |
| | HS105_HUMAN | Q92598 | | 548 | 1 | Heat shock protein 105 kDa |
| | HS105_HUMAN | Q92598 | | 548 | 1 | Heat shock protein 105 kDa |
| | HS71L_HUMAN | P34931 | | 228 | 6 | Heat shock 70 kDa protein |
| | | | | | | 1L |
| | HSP71_HUMAN | P08107 | | 226 | | Heat shock 70 kDa protein 1 |
| | HSP72_HUMAN | P54652 | | 229 | | Heat shock-related 70 kDa |
| | | | | | | protein 2 |
| | HSP76_HUMAN | P17066 | | 228 | | Heat shock 70 kDa protein 6 |
| | HSP77_HUMAN | P48741 | | 228 | | Putative heat shock 70 kDa |
| | | | | | | protein 7 |
| | HSP7C_HUMAN | P11142 | | 226 | | Heat shock cognate 71 kDa |
| | | | | | | protein |
| | HSP74_HUMAN | P34932 | | 728 | 1 | Heat shock 70 kDa protein 4 |
| | HSP7C_HUMAN | P11142 | | 81 | 1 | Heat shock cognate 71 kDa |
| | | | | | | protein |
| | HTF4_HUMAN | Q99081 | | 23 | 1 | Transcription factor 12 |
| 104 | HTSF1_HUMAN | O43719 | AGGEPDSLGQQP | 34 | 1 | HIV Tat-specific factor 1 |
| | | | TDTPYEWDLDKK | | | |
| 105 | HTSF1_HUMAN | O43719 | AGGEPDSLGQQP | 34 | 1 | HIV Tat-specific factor 1 |
| | | | TDTPYEWDLDKK | | | |
| | | | AWFPK | | | |
| 106 | HTSF1_HUMAN | O43719 | GASSSTANVEDV | 81 | 1 | HIV Tat-specific factor 1 |
| | | | HAR | | | |
| | HTSF1_HUMAN | O43719 | | 40 | 1 | HIV Tat-specific factor 1 |
| | HUWE1_HUMAN | Q7Z6Z7 | | 2360 | 1 | E3 ubiquitin-protein ligase |
| | | | | | | HUWE1 |
| 107 | HUWE1_HUMAN | Q7Z6Z7 | GLPEEQPQTTK | 3665 | 1 | E3 ubiquitin-protein ligase |
| | | | | | | HUWE1 |
| 108 | HUWE1_HUMAN | Q7Z6Z7 | MNASPLVR | 2474 | 1 | E3 ubiquitin-protein ligase |
| | | | | | | HUWE1 |
| 109 | HUWE1_HUMAN | Q7Z6Z7 | SAVAISGADSR | 2931 | 1 | E3 ubiquitin-protein ligase |
| | | | | | | HUWE1 |
| | HUWE1_HUMAN | Q7Z6Z7 | | 2018 | 1 | E3 ubiquitin-protein ligase |
| | | | | | | HUWE1 |
| 110 | HUWE1_HUMAN | Q7Z6Z7 | SVLAVMPPDIAAE | 3080 | 1 | E3 ubiquitin-protein ligase |
| | | | AQALR | | | HUWE1 |
| | I2BP2_HUMAN | Q7Z5L9 | | 496 | 1 | Interferon regulatory factor |
| | | | | | | 2-binding protein 2 |
| | I5P2_HUMAN | P32019 | | 264 | 1 | Type II inositol-1,4,5- |
| | | | | | | trisphosphate 5-phosphatase |
| | IASPP_HUMAN | Q8WUF5 | | 295 | 1 | RelA-associated inhibitor |
| 111 | ICAL_HUMAN | P20810 | ALSSDFTCGSPTA | 234 | 1 | Calpastatin |
| | | | AGK | | | |
| 112 | ICAL_HUMAN | P20810 | ALSSDFTCGSPTA | 234 | 1 | Calpastatin |
| | | | AGKK | | | |
| | ICAL_HUMAN | P20810 | | 514 | 1 | Calpastatin |
| | ICAL_HUMAN | P20810 | | 349 | 1 | Calpastatin |
| | ICAL_HUMAN | P20810 | | 660 | 1 | Calpastatin |
| | IF2BL_HUMAN | A6NK07 | | 119 | 2 | Eukaryotic translation |
| | | | | | | initiation factor 2 subunit 2- |
| | | | | | | like protein |
| | IF2B_HUMAN | P20042 | | 119 | | Eukaryotic translation |
| | | | | | | initiation factor 2 subunit 2 |
| | IF2P_HUMAN | O60841 | | 21 | 1 | Eukaryotic translation |
| | | | | | | initiation factor 5B |
| | IF2P_HUMAN | O60841 | | 21 | 1 | Eukaryotic translation |
| | | | | | | initiation factor 5B |
| | IF2P_HUMAN | O60841 | | 21 | 1 | Eukaryotic translation |
| | | | | | | initiation factor 5B |
| | IF4A2_HUMAN | Q14240 | | 22 | 1 | Eukaryotic initiation factor |
| | | | | | | 4A-II |
| | IF4A2_HUMAN | Q14240 | | 22 | 1 | Eukaryotic initiation factor |
| | | | | | | 4A-II |
| | IF4B_HUMAN | P23588 | | 60 | 1 | Eukaryotic translation |
| | | | | | | initiation factor 4B |
| | IF4B_HUMAN | P23588 | | 51 | 1 | Eukaryotic translation |
| | | | | | | initiation factor 4B |
| | IF4G1_HUMAN | Q04637 | | 533 | 1 | Eukaryotic translation |
| | | | | | | initiation factor 4 gamma 1 |
| | IF4G1_HUMAN | Q04637 | | 666 | 1 | Eukaryotic translation |
| | | | | | | initiation factor 4 gamma 1 |
| | IF4G1_HUMAN | Q04637 | | 415 | 1 | Eukaryotic translation |
| | | | | | | initiation factor 4 gamma 1 |
| 113 | IF4G2_HUMAN | P78344 | SSSAPSKEQLEQEK | 793 | 1 | Eukaryotic translation |
| | | | | | | initiation factor 4 gamma 2 |
| 114 | IF4G2_HUMAN | P78344 | SSSAPSKEQLEQE | 793 | 1 | Eukaryotic translation |
| | | | KQLLLSFKPVMQK | | | initiation factor 4 gamma 2 |
| | IF4G3_HUMAN | O43432 | | 479 | 1 | Eukaryotic translation |
| | | | | | | initiation factor 4 gamma 3 |
| | IF4G3_HUMAN | O43432 | | 479 | 1 | Eukaryotic translation |
| | | | | | | initiation factor 4 gamma 3 |
| 115 | IF4H_HUMAN | Q15056 | SLKEALTYDGAL | 94 | 1 | Eukaryotic translation |
| | | | LGDR | | | initiation factor 4H |
| | IF5A1_HUMAN | P63241 | | 97 | 1 | Eukaryotic translation |
| | | | | | | initiation factor 5A-1 |
| | IF5A1_HUMAN | P63241 | | 12 | 2 | Eukaryotic translation |
| | | | | | | initiation factor 5A-1 |
| | IF5AL_HUMAN | Q6IS14 | | 12 | | Eukaryotic translation |
| | | | | | | initiation factor 5A-1-like |
| | IF5A1_HUMAN | P63241 | | 12 | 2 | Eukaryotic translation |
| | | | | | | initiation factor 5A-1 |
| | IF5AL_HUMAN | Q6IS14 | | 12 | | Eukaryotic translation |
| | | | | | | initiation factor 5A-1-like |
| | IF5A1_HUMAN | P63241 | | 12 | 2 | Eukaryotic translation |
| | | | | | | initiation factor 5A-1 |
| | IF5AL_HUMAN | Q6IS14 | | 12 | | Eukaryotic translation |
| | | | | | | initiation factor 5A-1-like |
| | IF5A1_HUMAN | P63241 | | 7 | 2 | Eukaryotic translation |
| | | | | | | initiation factor 5A-1 |
| | IF5AL_HUMAN | Q6IS14 | | 7 | | Eukaryotic translation |
| | | | | | | initiation factor 5A-1-like |
| | IF5A1_HUMAN | P63241 | | 12 | 2 | Eukaryotic translation |
| | | | | | | initiation factor 5A-1 |
| | IF5AL_HUMAN | Q6IS14 | | 12 | | Eukaryotic translation |
| | | | | | | initiation factor 5A-1-like |
| | IF5A1_HUMAN | P63241 | | 7 | 2 | Eukaryotic translation |
| | | | | | | initiation factor 5A-1 |
| | IF5AL_HUMAN | Q6IS14 | | 7 | | Eukaryotic translation |
| | | | | | | initiation factor 5A-1-like |
| | IF5A1_HUMAN | P63241 | | 7 | 2 | Eukaryotic translation |
| | | | | | | initiation factor 5A-1 |
| | IF5AL_HUMAN | Q6IS14 | | 7 | | Eukaryotic translation |
| | | | | | | initiation factor 5A-1-like |
| | IF5A1_HUMAN | P63241 | | 7 | 2 | Eukaryotic translation |
| | | | | | | initiation factor 5A-1 |
| | IF5AL_HUMAN | Q6IS14 | | 7 | | Eukaryotic translation |
| | | | | | | initiation factor 5A-1-like |
| | IF5A2_HUMAN | Q9GZV4 | | 7 | 1 | Eukaryotic translation |
| | | | | | | initiation factor 5A-2 |
| | IF5A2_HUMAN | Q9GZV4 | | 7 | 1 | Eukaryotic translation |
| | | | | | | initiation factor 5A-2 |
| | IKBB_HUMAN | Q15653 | | 160 | 1 | NF-kappa-B inhibitor beta |
| 116 | IKBL2_HUMAN | Q96HA7 | GLTPQLEEDEELQ | 499 | 1 | NF-kappa-B inhibitor-like |
| | | | GHLGR | | | protein 2 |
| 117 | IKBL2_HUMAN | Q96HA7 | GLTPQLEEDEELQ | 499 | 1 | NF-kappa-B inhibitor-like |
| | | | GHLGRR | | | protein 2 |
| | IKZF1_HUMAN | Q13422 | | 368 | 1 | DNA-binding protein Ikaros |
| | IKZF2_HUMAN | Q9UKS7 | | 8 | 1 | Zinc finger protein Helios |
| | IKZF5_HUMAN | Q9H5V7 | | 226 | 1 | Zinc finger protein Pegasus |
| 118 | ILF3_HUMAN | Q12906 | GSGIYDPCEKEAT | 288 | 1 | Interleukin enhancer-binding |
| | | | DAIGHLDR | | | factor 3 |
| | ILF3_HUMAN | Q12906 | | 440 | 1 | Interleukin enhancer-binding |
| | | | | | | factor 3 |
| | ILF3_HUMAN | Q12906 | | 440 | 1 | Interleukin enhancer-binding |
| | | | | | | factor 3 |
| | ILKAP_HUMAN | Q9H0C8 | | 40 | 1 | Integrin-linked kinase- |
| | | | | | | associated serine/threonine |
| | | | | | | phosphatase 2C |
| | IMA1_HUMAN | P52294 | | 65 | 1 | Importin subunit alpha-1 |
| | IMA1_HUMAN | P52294 | | 65 | 1 | Importin subunit alpha-1 |
| | IMA7_HUMAN | O60684 | | 70 | 1 | Importin subunit alpha-7 |
| 119 | IMDH2_HUMAN | P12268 | CFLEEIMTK | 173 | 1 | Inosine-5′-monophosphate |
| | | | | | | dehydrogenase 2 |
| | IN80D_HUMAN | Q53TQ3 | | 679 | 1 | INO80 complex subunit D |
| 120 | INF2_HUMAN | Q27J81 | AVTPGPQPTLEQL | 1052 | 1 | Inverted formin-2 |
| | | | EEGGPRPLER | | | |
| 121 | INF2_HUMAN | Q27J81 | AVTPGPQPTLEQL | 1052 | 1 | Inverted formin-2 |
| | | | EEGGPRPLERR | | | |
| | INF2_HUMAN | Q27J81 | | 1147 | 1 | Inverted formin-2 |
| | IPO9_HUMAN | Q96P70 | | 964 | 1 | Importin-9 |
| | IQEC1_HUMAN | Q6DN90 | | 235 | 1 | IQ motif and SEC7 domain- |
| | | | | | | containing protein 1 |
| 122 | IQGA1_HUMAN | P46940 | GLGVARPHYGSV | 9 | 1 | Ras GTPase-activating-like |
| | | | LDNER | | | protein IQGAP1 |
| 123 | IQGA1_HUMAN | P46940 | GLGVARPHYGSV | 9 | 1 | Ras GTPase-activating-like |
| | | | LDNERLTAEEMD | | | protein IQGAP1 |
| | | | ER | | | |
| | IRF2_HUMAN | P14316 | | 238 | 1 | Interferon regulatory factor 2 |
| | IRS4_HUMAN | O14654 | | 717 | 1 | Insulin receptor substrate 4 |
| 124 | ISY1_HUMAN | Q9ULR0 | GVIVPLEQEYEK | 168 | 1 | Pre-mRNA-splicing factor |
| | | | | | | ISY1 homolog |
| 125 | ISY1_HUMAN | Q9ULR0 | GVIVPLEQEYEKK | 168 | 1 | Pre-mRNA-splicing factor |
| | | | | | | ISY1 homolog |
| | IWS1_HUMAN | Q96ST2 | | 348 | 1 | Protein IWS1 homolog |
| | IWS1_HUMAN | Q96ST2 | | 348 | 1 | Protein IWS1 homolog |
| 126 | JHD3C_HUMAN | Q9H3R0 | GAEVPNPDSVTD | 397 | 1 | JmjC domain-containing |
| | | | DLK | | | histone demethylation |
| | | | | | | protein 3C |
| 127 | JHD3C_HUMAN | Q9H3R0 | GAEVPNPDSVTD | 397 | 1 | JmjC domain-containing |
| | | | DLKVSEK | | | histone demethylation |
| | | | | | | protein 3C |
| | JIP4_HUMAN | O60271 | | 214 | 1 | C-jun-amino-terminal |
| | | | | | | kinase-interacting protein 4 |
| | JIP4_HUMAN | O60271 | | 6 | 1 | C-jun-amino-terminal |
| | | | | | | kinase-interacting protein 4 |
| | JIP4_HUMAN | O60271 | | 6 | 1 | C-jun-amino-terminal |
| | | | | | | kinase-interacting protein 4 |
| | JIP4_HUMAN | O60271 | | 285 | 1 | C-jun-amino-terminal |
| | | | | | | kinase-interacting protein 4 |
| | JKIP1_HUMAN | Q96N16 | | 18 | 1 | Janus kinase and |
| | | | | | | microtubule-interacting |
| | | | | | | protein 1 |
| 128 | JKIP1_HUMAN | Q96N16 | AVQMANEELR | 18 | 1 | Janus kinase and |
| | | | | | | microtubule-interacting |
| | | | | | | protein 1 |
| | JMY_HUMAN | Q8N9B5 | | 723 | 1 | Junction-mediating and - |
| | | | | | | regulatory protein |
| | JMY_HUMAN | Q8N9B5 | | 723 | 1 | Junction-mediating and - |
| | | | | | | regulatory protein |
| 129 | JOSD3_HUMAN | Q9H5J8 | HVTSDAVELANR | 11 | 1 | Protein JOSD3 |
| 130 | JSPR1_HUMAN | Q96MG2 | GGLGSCQALEDH | 13 | 1 | Junctional sarcoplasmic |
| | | | SALAETQEDR | | | reticulum protein 1 |
| | K0174_HUMAN | P53990 | | 198 | 1 | Uncharacterized protein |
| | | | | | | KIAA0174 |
| | K0174_HUMAN | P53990 | | 198 | 1 | Uncharacterized protein |
| | | | | | | KIAA0174 |
| | K0232_HUMAN | Q92628 | | 557 | 1 | Uncharacterized protein |
| | | | | | | KIAA0232 |
| | K0515_HUMAN | Q5JSZ5 | | 1083 | 1 | Uncharacterized protein |
| | | | | | | KIAA0515 |
| | K0515_HUMAN | Q5JSZ5 | | 1236 | 1 | Uncharacterized protein |
| | | | | | | KIAA0515 |
| | K0831_HUMAN | Q6ZNE5 | | 29 | 1 | Uncharacterized protein |
| | | | | | | KIAA0831 |
| | K0831_HUMAN | Q6ZNE5 | | 227 | 1 | Uncharacterized protein |
| | | | | | | KIAA0831 |
| | K1462_HUMAN | Q9P266 | | 1180 | 1 | Uncharacterized protein |
| | | | | | | KIAA1462 |
| 131 | K1543_HUMAN | Q9P1Y5 | GSPAGAEDSLEEE | 862 | 1 | Uncharacterized protein |
| | | | ASSEGEPR | | | KIAA1543 |
| 132 | K1627_HUMAN | Q9HCE5 | SIGAVLNSKDEQR | 30 | 1 | Methyltransferase-like |
| | | | | | | protein KIAA1627 |
| 133 | K1627_HUMAN | Q9HCE5 | SIGAVLNSKDEQR | 30 | 1 | Methyltransferase-like |
| | | | EIAETR | | | protein KIAA1627 |
| 134 | K1627_HUMAN | Q9HCE5 | SIGAVLNSK | 30 | 1 | Methyltransferase-like |
| | | | | | | protein KIAA1627 |
| | K1704_HUMAN | Q8IXQ4 | | 89 | 1 | Uncharacterized protein |
| | | | | | | KIAA1704 |
| 135 | K1967_HUMAN | Q8N163 | AGAEPITADSDPA | 293 | 1 | Protein KIAA1967 |
| | | | YSSK | | | |
| | K1967_HUMAN | Q8N163 | | 769 | 1 | Protein KIAA1967 |
| | K1967_HUMAN | Q8N163 | | 619 | 1 | Protein KIAA1967 |
| 136 | KHDR1_HUMAN | Q07666 | ATVGGPAPTPLLP | 76 | 1 | KH domain-containing, |
| | | | PSATASVK | | | RNA-binding, signal |
| | | | | | | transduction-associated |
| | | | | | | protein 1 |
| | KI67_HUMAN | P46013 | | 2148 | 1 | Antigen KI-67 |
| | KI67_HUMAN | P46013 | | 411 | 1 | Antigen KI-67 |
| | KI67_HUMAN | P46013 | | 174 | 1 | Antigen KI-67 |
| | KIF15_HUMAN | Q9NS87 | | 1134 | 1 | Kinesin-like protein KIF15 |
| | KKCC1_HUMAN | Q8N5S9 | | 33 | 1 | Calcium/calmodulin- |
| | | | | | | dependent protein kinase |
| | | | | | | kinase 1 |
| | KLF12_HUMAN | Q9Y4X4 | | 74 | 1 | Krueppel-like factor 12 |
| 137 | KPYM_HUMAN | P14618 | GADCIMLSGETA | 355 | 1 | Pyruvate kinase isozymes |
| | | | KGDYPLEAVR | | | M1/M2 |
| 138 | KPYM_HUMAN | P14618 | GADCIMLSGETAK | 355 | 2 | Pyruvate kinase isozymes |
| | | | | | | M1/M2 |
| 138 | KPYR_HUMAN | P30613 | GADCIMLSGETAK | 398 | | Pyruvate kinase isozymes |
| | | | | | | R/L |
| | KRI1_HUMAN | Q8N9T8 | | 313 | 1 | Protein KRI1 homolog |
| 139 | KRR1_HUMAN | Q13601 | GWKEPAFSK | 39 | 1 | KRR1 small subunit |
| | | | | | | processome component |
| | | | | | | homolog |
| 140 | KRR1_HUMAN | Q13601 | GWKEPAFSKEDN | 39 | 1 | KRR1 small subunit |
| | | | PR | | | processome component |
| | | | | | | homolog |
| | KS6A4_HUMAN | O75676 | | 378 | 1 | Ribosomal protein S6 kinase |
| | | | | | | alpha-4 |
| | KU86_HUMAN | P13010 | | 456 | 1 | ATP-dependent DNA |
| | | | | | | helicase 2 subunit 2 |
| | KU86_HUMAN | P13010 | | 456 | 1 | ATP-dependent DNA |
| | | | | | | helicase 2 subunit 2 |
| | KU86_HUMAN | P13010 | | 557 | 1 | ATP-dependent DNA |
| | | | | | | helicase 2 subunit 2 |
| | LAGE3_HUMAN | Q14657 | | 29 | 1 | L antigen family member 3 |
| | LAMB1_HUMAN | P07942 | | 1359 | 1 | Laminin subunit beta-1 |
| | LAP2A_HUMAN | P42166 | | 487 | 1 | Lamina-associated |
| | | | | | | polypeptide 2, isoform alpha |
| | LAP2A_HUMAN | P42166 | | 442 | 1 | Lamina-associated |
| | | | | | | polypeptide 2, isoform alpha |
| 141 | LAP4_HUMAN | Q14160 | AALEVSPGVIANP | 1198 | 1 | Protein LAP4 |
| | | | FAAGIGHR | | | |
| | LAP4_HUMAN | Q14160 | | 502 | 1 | Protein LAP4 |
| | LAP4_HUMAN | Q14160 | | 636 | 1 | Protein LAP4 |
| 142 | LARP1_HUMAN | Q6PKG0 | AINWPTPGEIAHK | 173 | 1 | La-related protein 1 |
| 143 | LARP1_HUMAN | Q6PKG0 | FSQLLNCPEFVPR | 496 | 1 | La-related protein 1 |
| 144 | LARP4_HUMAN | Q71RC2 | GLNQTTIPVSPPST | 574 | 1 | La-related protein 4 |
| | | | TKPSR | | | |
| | LARP5_HUMAN | Q92615 | | 136 | 1 | La-related protein 5 |
| | LAT_HUMAN | O43561 | | 168 | 1 | Linker for activation of T- |
| | | | | | | cells family member 1 |
| 145 | LCAP_HUMAN | Q9UIQ6 | LAKEPCLHPLEPD | 30 | 1 | Leucyl-cystinyl |
| | | | EVEYEPR | | | aminopeptidase |
| | LCORL_HUMAN | Q8N3X6 | | 230 | 2 | Ligand-dependent nuclear |
| | | | | | | receptor corepressor-like |
| | | | | | | protein |
| | LCOR_HUMAN | Q96JN0 | | 81 | | Ligand-dependent |
| | | | | | | corepressor |
| | LIMA1_HUMAN | Q9UHB6 | | 346 | 1 | LIM domain and actin- |
| | | | | | | binding protein 1 |
| | LIN37_HUMAN | Q96GY3 | | 24 | 1 | Protein lin-37 homolog |
| | LIN7C_HUMAN | Q9NUP9 | | 63 | 1 | Lin-7 homolog C |
| 146 | LIPA1_HUMAN | Q13136 | GVLDINHEQENTP | 219 | 1 | Liprin-alpha-1 |
| | | | STSGK | | | |
| 147 | LIPA1_HUMAN | Q13136 | GVLDINHEQENTP | 219 | 1 | Liprin-alpha-1 |
| | | | STSGKR | | | |
| | LIPB2_HUMAN | Q8ND30 | | 32 | 1 | Liprin-beta-2 |
| | LMNB1_HUMAN | P20700 | | 147 | 1 | Lamin-B1 |
| | LMO7_HUMAN | Q8WWI1 | | 963 | 1 | LIM domain only protein 7 |
| | LMTK2_HUMAN | Q8IWU2 | | 901 | 1 | Serine/threonine-protein |
| | | | | | | kinase LMTK2 |
| | LNP_HUMAN | Q9C0E8 | | 369 | 1 | Protein lunapark |
| | LPP_HUMAN | Q93052 | | 404 | 1 | Lipoma-preferred partner |
| | LPP_HUMAN | Q93052 | | 404 | 1 | Lipoma-preferred partner |
| | LRBA_HUMAN | P50851 | | 1757 | 1 | Lipopolysaccharide- |
| | | | | | | responsive and beige-like |
| | | | | | | anchor protein |
| 148 | LRBA_HUMAN | P50851 | SAQASDMGGESP | 1757 | 1 | Lipopolysaccharide- |
| | | | GSR | | | responsive and beige-like |
| | | | | | | anchor protein |
| | LRBA_HUMAN | P50851 | | 1785 | 1 | Lipopolysaccharide- |
| | | | | | | responsive and beige-like |
| | | | | | | anchor protein |
| | LRBA_HUMAN | P50851 | | 1785 | 1 | Lipopolysaccharide- |
| | | | | | | responsive and beige-like |
| | | | | | | anchor protein |
| 149 | LRC47_HUMAN | Q8N1G4 | AVSGQLPDPTTNP | 526 | 1 | Leucine-rich repeat- |
| | | | SAGK | | | containing protein 47 |
| 150 | LRC47_HUMAN | Q8N1G4 | AVSGQLPDPTTNP | 526 | 1 | Leucine-rich repeat- |
| | | | SAGKDGPSLLVV | | | containing protein 47 |
| | | | EQVR | | | |
| | LRCH1_HUMAN | Q9Y2L9 | | 406 | 1 | Leucine-rich repeat and |
| | | | | | | calponin homology domain- |
| | | | | | | containing protein 1 |
| | LRCH1_HUMAN | Q9Y2L9 | | 406 | 1 | Leucine-rich repeat and |
| | | | | | | calponin homology domain- |
| | | | | | | containing protein 1 |
| | LRCH2_HUMAN | Q5VUJ6 | | 604 | 1 | Leucine-rich repeat and |
| | | | | | | calponin homology domain- |
| | | | | | | containing protein 2 |
| | LRCH3_HUMAN | Q96II8 | | 643 | 1 | Leucine-rich repeat and |
| | | | | | | calponin homology domain- |
| | | | | | | containing protein 3 |
| | LRCH4_HUMAN | O75427 | | 359 | 1 | Leucine-rich repeat and |
| | | | | | | calponin homology domain- |
| | | | | | | containing protein 4 |
| 151 | LRMP_HUMAN | Q12912 | SVVSPLPVTTVK | 182 | 1 | Lymphoid-restricted |
| | | | | | | membrane protein |
| | LRRF1_HUMAN | Q32MZ4 | | 416 | 1 | Leucine-rich repeat |
| | | | | | | flightless-interacting protein 1 |
| | LRRF2_HUMAN | Q9Y608 | | 532 | 1 | Leucine-rich repeat |
| | | | | | | flightless-interacting protein 2 |
| | LSM11_HUMAN | P83369 | | 306 | 1 | U7 snRNA-associated Sm- |
| | | | | | | like protein LSm11 |
| | LSM3_HUMAN | P62310 | | 7 | 1 | U6 snRNA-associated Sm- |
| | | | | | | like protein LSm3 |
| | LSP1_HUMAN | P33241 | | 103 | 1 | Lymphocyte-specific protein 1 |
| 152 | LTV1_HUMAN | Q96GA3 | SAGLLSDEDCMS | 206 | 1 | Protein LTV1 homolog |
| | | | VPGKTHR | | | |
| | LYRIC_HUMAN | Q86UE4 | | 184 | 1 | Protein LYRIC |
| 153 | M6PBP_HUMAN | O60664 | GFDVASVQQQR | 220 | 1 | Mannose-6-phosphate |
| | | | | | | receptor-binding protein 1 |
| | M6PBP_HUMAN | O60664 | | 10 | 1 | Mannose-6-phosphate |
| | | | | | | receptor-binding protein 1 |
| | M6PBP_HUMAN | O60664 | | 223 | 1 | Mannose-6-phosphate |
| | | | | | | receptor-binding protein 1 |
| | MA7D1_HUMAN | Q3KQU3 | | 571 | 1 | MAP7 domain-containing |
| | | | | | | protein 1 |
| 154 | MA7D1_HUMAN | Q3KQU3 | AAVLTSPPAPAPP | 571 | 1 | MAP7 domain-containing |
| | | | VTPSKPMAGTTD | | | protein 1 |
| | | | REEATR | | | |
| | MACF1_HUMAN | Q9UPN3 | | 1524 | 1 | Microtubule-actin cross- |
| | | | | | | linking factor 1, isoforms |
| | | | | | | 1/2/3/5 |
| | MACF1_HUMAN | Q9UPN3 | | 3021 | 2 | Microtubule-actin cross- |
| | | | | | | linking factor 1, isoforms |
| | | | | | | 1/2/3/5 |
| | MACF4_HUMAN | Q96PK2 | | 3523 | | Microtubule-actin cross- |
| | | | | | | linking factor 1, isoform 4 |
| | MACF1_HUMAN | Q9UPN3 | | 3021 | 2 | Microtubule-actin cross- |
| | | | | | | linking factor 1, isoforms |
| | | | | | | 1/2/3/5 |
| | MACF4_HUMAN | Q96PK2 | | 3523 | | Microtubule-actin cross- |
| | | | | | | linking factor 1, isoform 4 |
| 155 | MACF1_HUMAN | Q9UPN3 | GYMGVNQAPEKL | 1727 | 2 | Microtubule-actin cross- |
| | | | DKQCEMMK | | | linking factor 1, isoforms |
| | | | | | | 1/2/3/5 |
| 155 | MACF4_HUMAN | Q96PK2 | GYMGVNQAPEKL | 2229 | | Microtubule-actin cross- |
| | | | DKQCEMMK | | | linking factor 1, isoform 4 |
| | MACF1_HUMAN | Q9UPN3 | | 1727 | 2 | Microtubule-actin cross- |
| | | | | | | linking factor 1, isoforms |
| | | | | | | 1/2/3/5 |
| | MACF4_HUMAN | Q96PK2 | | 2229 | | Microtubule-actin cross- |
| | | | | | | linking factor 1, isoform 4 |
| | MACF1_HUMAN | Q9UPN3 | | 1727 | 2 | Microtubule-actin cross- |
| | | | | | | linking factor 1, isoforms |
| | | | | | | 1/2/3/5 |
| | MACF4_HUMAN | Q96PK2 | | 2229 | | Microtubule-actin cross- |
| | | | | | | linking factor 1, isoform 4 |
| 156 | MACF1_HUMAN | Q9UPN3 | GYMGVNQAPEKL | 1727 | 2 | Microtubule-actin cross- |
| | | | DK | | | linking factor 1, isoforms |
| | | | | | | 1/2/3/5 |
| 156 | MACF4_HUMAN | Q96PK2 | GYMGVNQAPEKL | 2229 | | Microtubule-actin cross- |
| | | | DK | | | linking factor 1, isoform 4 |
| 157 | MACF1_HUMAN | Q9UPN3 | GYMGVNQAPEK | 1727 | 2 | Microtubule-actin cross- |
| | | | | | | linking factor 1, isoforms |
| | | | | | | 1/2/3/5 |
| 157 | MACF4_HUMAN | Q96PK2 | GYMGVNQAPEK | 2229 | | Microtubule-actin cross- |
| | | | | | | linking factor 1, isoform 4 |
| 158 | MADD_HUMAN | Q8WXG6 | SVIGVSPAVMIR | 1178 | 1 | MAP kinase-activating death |
| | | | | | | domain protein |
| | MAGD1_HUMAN | Q9Y5V3 | | 223 | 1 | Melanoma-associated |
| | | | | | | antigen D1 |
| 159 | MAGG1_HUMAN | Q96MG7 | GFAEEAPSTSR | 42 | 1 | Melanoma-associated |
| | | | | | | antigen G1 |
| 160 | MAGG1_HUMAN | Q96MG7 | GFAEEAPSTSRGP | 42 | 1 | Melanoma-associated |
| | | | GGSQGSQGPSPQ | | | antigen G1 |
| | | | GAR | | | |
| | MAOM_HUMAN | P23368 | | 380 | 1 | NAD-dependent malic |
| | | | | | | enzyme, mitochondrial |
| 161 | MAP1A_HUMAN | P78559 | SVVAAVQEGAAE | 1885 | 1 | Microtubule-associated |
| | | | LEGGPYSPLGK | | | protein 1A |
| 162 | MAP1A_HUMAN | P78559 | SVVAAVQEGAAE | 1885 | 1 | Microtubule-associated |
| | | | LEGGPYSPLGKD | | | protein 1A |
| | | | YR | | | |
| 163 | MAP1A_HUMAN | P78559 | SVVAAVQEGAAE | 1885 | 1 | Microtubule-associated |
| | | | LEGGPYSPLGKD | | | protein 1A |
| | | | YRK | | | |
| | MAP4_HUMAN | P27816 | | 9 | 1 | Microtubule-associated |
| | | | | | | protein 4 |
| | MAP4_HUMAN | P27816 | | 9 | 1 | Microtubule-associated |
| | | | | | | protein 4 |
| | MAP4_HUMAN | P27816 | | 250 | 1 | Microtubule-associated |
| | | | | | | protein 4 |
| | MAP4_HUMAN | P27816 | | 152 | 1 | Microtubule-associated |
| | | | | | | protein 4 |
| | MAP4_HUMAN | P27816 | | 328 | 1 | Microtubule-associated |
| | | | | | | protein 4 |
| | MAP4_HUMAN | P27816 | | 47 | 1 | Microtubule-associated |
| | | | | | | protein 4 |
| | MAP9_HUMAN | Q49MG5 | | 120 | 1 | Microtubule-associated |
| | | | | | | protein 9 |
| | MARE1_HUMAN | Q15691 | | 117 | 1 | Microtubule-associated |
| | | | | | | protein RP/EB family |
| | | | | | | member 1 |
| | MARK1_HUMAN | Q9P0L2 | | 23 | 1 | Serine/threonine-protein |
| | | | | | | kinase MARK1 |
| 164 | MATR3_HUMAN | P43243 | GQSDENKDDYTIP | 764 | 1 | Matrin-3 |
| | | | DEYR | | | |
| 165 | MATR3_HUMAN | P43243 | LANLGDVASDGK | 681 | 1 | Matrin-3 |
| 166 | MATR3_HUMAN | P43243 | LANLGDVASDGKK | 681 | 1 | Matrin-3 |
| 167 | MATR3_HUMAN | P43243 | SFDDRGPSLNPVL | 188 | 1 | Matrin-3 |
| | | | DYDHGSR | | | |
| 168 | MATR3_HUMAN | P43243 | YYTTTPALVFGKP | 453 | 1 | Matrin-3 |
| | | | VR | | | |
| | MATR3_HUMAN | P43243 | | 704 | 1 | Matrin-3 |
| 169 | MATR3_HUMAN | P43243 | LANLGDVASDGK | 681 | 1 | Matrin-3 |
| | | | KEPSDK | | | |
| | MAVS_HUMAN | Q7Z434 | | 491 | 1 | Mitochondrial antiviral- |
| | | | | | | signaling protein |
| | MAVS_HUMAN | Q7Z434 | | 491 | 1 | Mitochondrial antiviral- |
| | | | | | | signaling protein |
| | MAX_HUMAN | P61244 | | 49 | 1 | Protein max |
| | MBB1A_HUMAN | Q9BQG0 | | 750 | 1 | Myb-binding protein 1A |
| | MCM2_HUMAN | P49736 | | 89 | 1 | DNA replication licensing |
| | | | | | | factor MCM2 |
| | MCM2_HUMAN | P49736 | | 69 | 1 | DNA replication licensing |
| | | | | | | factor MCM2 |
| 170 | MCM3_HUMAN | P25205 | SYDPYDFSDTEEE | 704 | 1 | DNA replication licensing |
| | | | MPQVHTPK | | | factor MCM3 |
| 171 | MCM4_HUMAN | P33991 | GAAAEDIVASEQS | 133 | 1 | DNA replication licensing |
| | | | LGQK | | | factor MCM4 |
| 172 | MCM5_HUMAN | P33992 | SFGGDAQADEGQ | 14 | 1 | DNA replication licensing |
| | | | ARK | | | factor MCM5 |
| 173 | MCM5_HUMAN | P33992 | SFGGDAQADEGQ | 14 | 1 | DNA replication licensing |
| | | | AR | | | factor MCM5 |
| 174 | MCM6_HUMAN | Q14566 | GYETEGIRGLR | 275 | 1 | DNA replication licensing |
| | | | | | | factor MCM6 |
| 175 | MCM6_HUMAN | Q14566 | GYETEGIR | 275 | 1 | DNA replication licensing |
| | | | | | | factor MCM6 |
| | MDC1_HUMAN | Q14676 | | 1036 | 1 | Mediator of DNA damage |
| | | | | | | checkpoint protein 1 |
| | MDC1_HUMAN | Q14676 | | 1036 | 1 | Mediator of DNA damage |
| | | | | | | checkpoint protein 1 |
| | MDN1_HUMAN | Q9NU22 | | 5128 | 1 | Midasin |
| | MED14_HUMAN | O60244 | | 995 | 1 | Mediator of RNA |
| | | | | | | polymerase II transcription |
| | | | | | | subunit 14 |
| | MED1_HUMAN | Q15648 | | 931 | 1 | Mediator of RNA |
| | | | | | | polymerase II transcription |
| | | | | | | subunit 1 |
| | MED1_HUMAN | Q15648 | | 1485 | 1 | Mediator of RNA |
| | | | | | | polymerase II transcription |
| | | | | | | subunit 1 |
| | MED26_HUMAN | O95402 | | 408 | 1 | Mediator of RNA |
| | | | | | | polymerase II transcription |
| | | | | | | subunit 26 |
| | MEF2C_HUMAN | Q06413 | | 106 | 1 | Myocyte-specific enhancer |
| | | | | | | factor 2C |
| | MEF2C_HUMAN | Q06413 | | 106 | 1 | Myocyte-specific enhancer |
| | | | | | | factor 2C |
| | METK2_HUMAN | P31153 | | 40 | 1 | S-adenosylmethionine |
| | | | | | | synthetase isoform type-2 |
| | MEX3B_HUMAN | Q6ZN04 | | 355 | 1 | RNA-binding protein |
| | | | | | | MEX3B |
| | MGAP_HUMAN | Q8IWI9 | | 681 | 1 | MAX gene-associated |
| | | | | | | protein |
| | MGAP_HUMAN | Q8IWI9 | | 340 | 1 | MAX gene-associated |
| | | | | | | protein |
| | MGAP_HUMAN | Q8IWI9 | | 340 | 1 | MAX gene-associated |
| | | | | | | protein |
| | MGAP_HUMAN | Q8IWI9 | | 572 | 1 | MAX gene-associated |
| | | | | | | protein |
| | MIA3_HUMAN | Q5JRA6 | | 710 | 1 | Melanoma inhibitory activity |
| | | | | | | protein 3 |
| | MIER1_HUMAN | Q8N108 | | 52 | 1 | Mesoderm induction early |
| | | | | | | response protein 1 |
| | MINT_HUMAN | Q96T58 | | 1575 | 1 | Msx2-interacting protein |
| | MINT_HUMAN | Q96T58 | | 2008 | 1 | Msx2-interacting protein |
| | MINT_HUMAN | Q96T58 | | 2860 | 1 | Msx2-interacting protein |
| | MISSL_HUMAN | Q8NDC0 | | 10 | 1 | MAPK-interacting and |
| | | | | | | spindle-stabilizing protein- |
| | | | | | | like |
| 176 | MKL1_HUMAN | Q969V6 | ALSPEQPASHESQ | 122 | 1 | MKL/myocardin-like protein 1 |
| | | | GSVPSPLEAR | | | |
| | MKL2_HUMAN | Q9ULH7 | | 183 | 1 | MKL/myocardin-like protein 2 |
| 177 | MLL2_HUMAN | O14686 | ALYVACQGQPK | 387 | 1 | Histone-lysine N- |
| | | | | | | methyltransferase MLL2 |
| | MLL2_HUMAN | O14686 | | 1866 | 1 | Histone-lysine N- |
| | | | | | | methyltransferase MLL2 |
| | MLL3_HUMAN | Q8NEZ4 | | 2189 | 1 | Histone-lysine N- |
| | | | | | | methyltransferase MLL3 |
| | MOBL3_HUMAN | Q9Y3A3 | | 35 | 1 | Mps one binder kinase |
| | | | | | | activator-like 3 |
| | MOES_HUMAN | P26038 | | 115 | 1 | Moesin |
| | MORC3_HUMAN | Q14149 | | 665 | 1 | MORC family CW-type zinc |
| | | | | | | finger protein 3 |
| | MORC3_HUMAN | Q14149 | | 752 | 1 | MORC family CW-type zinc |
| | | | | | | finger protein 3 |
| | MOT1_HUMAN | P53985 | | 470 | 1 | Monocarboxylate transporter 1 |
| | MP2K1_HUMAN | Q02750 | | 283 | 1 | Dual specificity mitogen- |
| | | | | | | activated protein kinase |
| | | | | | | kinase 1 |
| 178 | MP2K1_HUMAN | Q02750 | GSAVNGTSSAET | 17 | 1 | Dual specificity mitogen- |
| | | | NLEALQK | | | activated protein kinase |
| | | | | | | kinase 1 |
| 179 | MP2K1_HUMAN | Q02750 | GSAVNGTSSAET | 17 | 1 | Dual specificity mitogen- |
| | | | NLEALQKK | | | activated protein kinase |
| | | | | | | kinase 1 |
| 180 | MPP10_HUMAN | O00566 | AALLAPEEIKEK | 546 | 1 | U3 small nucleolar |
| | | | | | | ribonucleoprotein protein |
| | | | | | | MPP10 |
| 181 | MPP10_HUMAN | O00566 | AALLAPEEIK | 546 | 1 | U3 small nucleolar |
| | | | | | | ribonucleoprotein protein |
| | | | | | | MPP10 |
| | MPP8_HUMAN | Q99549 | | 20 | 1 | M-phase phosphoprotein 8 |
| | MPP8_HUMAN | Q99549 | | 502 | 1 | M-phase phosphoprotein 8 |
| | MPP8_HUMAN | Q99549 | | 517 | 1 | M-phase phosphoprotein 8 |
| 182 | MRP_HUMAN | P49006 | AIEPAPPSQGAEAK | 64 | 1 | MARCKS-related protein |
| | MSPD2_HUMAN | Q8NHP6 | | 275 | 1 | Motile sperm domain- |
| | | | | | | containing protein 2 |
| | MTA70_HUMAN | Q86U44 | | 335 | 1 | N6-adenosine- |
| | | | | | | methyltransferase 70 kDa |
| | | | | | | subunit |
| | MYH10_HUMAN | P35580 | | 1310 | 1 | Myosin-10 |
| 183 | MYH10_HUMAN | P35580 | TTAAQQELR | 1161 | 1 | Myosin-10 |
| 184 | MYH11_HUMAN | P35749 | STATQQELR | 1161 | 1 | Myosin-11 |
| | MYH9_HUMAN | P35579 | | 1376 | 1 | Myosin-9 |
| 185 | MYH9_HUMAN | P35579 | STAAQQELR | 1154 | 1 | Myosin-9 |
| 186 | MYO9B_HUMAN | Q13459 | SLTSDKASVPIVL | 1704 | 1 | Myosin-IXb |
| | | | EK | | | |
| | MYPT1_HUMAN | O14974 | | 886 | 1 | Protein phosphatase 1 |
| | | | | | | regulatory subunit 12A |
| | N4BP1_HUMAN | O75113 | | 491 | 1 | NEDD4-binding protein 1 |
| | NACA_HUMAN | Q13765 | | 43 | 1 | Nascent polypeptide- |
| | | | | | | associated complex subunit |
| | | | | | | alpha |
| | NACA_HUMAN | Q13765 | | 43 | 1 | Nascent polypeptide- |
| | | | | | | associated complex subunit |
| | | | | | | alpha |
| | NADAP_HUMAN | Q9BWU0 | | 538 | 1 | Kanadaptin |
| | NADAP_HUMAN | Q9BWU0 | | 538 | 1 | Kanadaptin |
| | NADAP_HUMAN | Q9BWU0 | | 538 | 1 | Kanadaptin |
| | NAG_HUMAN | A2RRP1 | | 637 | 1 | Neuroblastoma-amplified |
| | | | | | | gene protein |
| | NAG_HUMAN | A2RRP1 | | 637 | 1 | Neuroblastoma-amplified |
| | | | | | | gene protein |
| | NAIF1_HUMAN | Q69YI7 | | 103 | 1 | Nuclear apoptosis-inducing |
| | | | | | | factor 1 |
| | NARF_HUMAN | Q9UHQ1 | | 292 | 1 | Nuclear prelamin A |
| | | | | | | recognition factor |
| | NARF_HUMAN | Q9UHQ1 | | 273 | 1 | Nuclear prelamin A |
| | | | | | | recognition factor |
| 187 | NASP_HUMAN | P49321 | KIEDVPAPSTSAD | 20 | 1 | Nuclear autoantigenic sperm |
| | | | KVESLDVDSEAK | | | protein |
| | NASP_HUMAN | P49321 | | 33 | 1 | Nuclear autoantigenic sperm |
| | | | | | | protein |
| | NASP_HUMAN | P49321 | | 33 | 1 | Nuclear autoantigenic sperm |
| | | | | | | protein |
| 188 | NCK1_HUMAN | P16333 | SASPADDSFVDPG | 89 | 1 | Cytoplasmic protein NCK1 |
| | | | ER | | | |
| | NCOA3_HUMAN | Q9Y6Q9 | | 1013 | 1 | Nuclear receptor coactivator 3 |
| | NCOA5_HUMAN | Q9HCD5 | | 381 | 1 | Nuclear receptor coactivator 5 |
| | NCOA5_HUMAN | Q9HCD5 | | 154 | 1 | Nuclear receptor coactivator 5 |
| | NCOA6_HUMAN | Q14686 | | 1462 | 1 | Nuclear receptor coactivator 6 |
| | NCOR1_HUMAN | O75376 | | 1827 | 1 | Nuclear receptor corepressor 1 |
| | NCOR1_HUMAN | O75376 | | 386 | 1 | Nuclear receptor corepressor 1 |
| | NCOR1_HUMAN | O75376 | | 556 | 1 | Nuclear receptor corepressor 1 |
| 189 | NCOR1_HUMAN | O75376 | AAASAPQMDVSK | 1827 | 1 | Nuclear receptor corepressor 1 |
| | NCOR1_HUMAN | O75376 | | 386 | 1 | Nuclear receptor corepressor 1 |
| | NCOR1_HUMAN | O75376 | | 556 | 1 | Nuclear receptor corepressor 1 |
| | NCOR2_HUMAN | Q9Y618 | | 378 | 1 | Nuclear receptor corepressor 2 |
| | NCOR2_HUMAN | Q9Y618 | | 1927 | 1 | Nuclear receptor corepressor 2 |
| | NDRG1_HUMAN | Q92597 | | 10 | 1 | Protein NDRG1 |
| 190 | NEB2_HUMAN | Q96SB3 | GTSLVGVTQSFA | 552 | 1 | Neurabin-2 |
| | | | ASVLR | | | |
| 191 | NED4L_HUMAN | Q96PU5 | AVAEQGHLPPPSA | 346 | 1 | E3 ubiquitin-protein ligase |
| | | | PAGR | | | NEDD4-like |
| | NEDD1_HUMAN | Q8NHV4 | | 435 | 1 | Protein NEDD1 |
| | NEDD4_HUMAN | P46934 | | 280 | 1 | E3 ubiquitin-protein ligase |
| | | | | | | NEDD4 |
| | NEK1_HUMAN | Q96PY6 | | 950 | 1 | Serine/threonine-protein |
| | | | | | | kinase Nek1 |
| | NEK4_HUMAN | P51957 | | 381 | 1 | Serine/threonine-protein |
| | | | | | | kinase Nek4 |
| | NEK9_HUMAN | Q8TD19 | | 842 | 1 | Serine/threonine-protein |
| | | | | | | kinase Nek9 |
| | NELFA_HUMAN | Q9H3P2 | | 300 | 1 | Negative elongation factor A |
| | NFAC1_HUMAN | O95644 | | 111 | 1 | Nuclear factor of activated T- |
| | | | | | | cells, cytoplasmic 1 |
| | NFAC2_HUMAN | Q13469 | | 67 | 1 | Nuclear factor of activated T- |
| | | | | | | cells, cytoplasmic 2 |
| | NFKB2_HUMAN | Q00653 | | 11 | 1 | Nuclear factor NF-kappa-B |
| | | | | | | p100 subunit |
| | NFRKB_HUMAN | Q6P4R8 | | 497 | 1 | Nuclear factor related to |
| | | | | | | kappa-B-binding protein |
| | NFRKB_HUMAN | Q6P4R8 | | 6 | 1 | Nuclear factor related to |
| | | | | | | kappa-B-binding protein |
| | NHERF_HUMAN | O14745 | | 5 | 1 | Ezrin-radixin-moesin- |
| | | | | | | binding phosphoprotein 50 |
| | NIPA_HUMAN | Q86WB0 | | 450 | 1 | Nuclear-interacting partner |
| | | | | | | of ALK |
| | NIPA_HUMAN | Q86WB0 | | 296 | 1 | Nuclear-interacting partner |
| | | | | | | of ALK |
| | NIPBL_HUMAN | Q6KC79 | | 473 | 1 | Nipped-B-like protein |
| | NIPBL_HUMAN | Q6KC79 | | 473 | 1 | Nipped-B-like protein |
| | NKTR_HUMAN | P30414 | | 960 | 1 | NK-tumor recognition |
| | | | | | | protein |
| | NOL1_HUMAN | P46087 | | 231 | 1 | Putative RNA |
| | | | | | | methyltransferase NOL1 |
| | NOL1_HUMAN | P46087 | | 208 | 1 | Putative RNA |
| | | | | | | methyltransferase NOL1 |
| 192 | NOL1_HUMAN | P46087 | GGLQINVDEEPFV | 208 | 1 | Putative RNA |
| | | | LPPAGEMEQDAQ | | | methyltransferase NOL1 |
| | | | APDLQR | | | |
| 193 | NOL1_HUMAN | P46087 | GGLQINVDEEPFV | 208 | 1 | Putative RNA |
| | | | LPPAGEMEQDAQ | | | methyltransferase NOL1 |
| | | | APDLQRVHKR | | | |
| 194 | NOL5_HUMAN | Q9Y2X3 | GLIPGVEPR | 125 | 1 | Nucleolar protein 5 |
| | NOP14_HUMAN | P78316 | | 320 | 1 | Nucleolar protein 14 |
| | NOP14_HUMAN | P78316 | | 320 | 1 | Nucleolar protein 14 |
| 195 | NP1L1_HUMAN | P55209 | GLVETPTGYIESL | 58 | 1 | Nucleosome assembly |
| | | | PR | | | protein 1-like 1 |
| 196 | NP1L1_HUMAN | P55209 | GLVETPTGYIESL | 58 | 1 | Nucleosome assembly |
| | | | PRVVKR | | | protein 1-like 1 |
| | NP1L1_HUMAN | P55209 | | 184 | 1 | Nucleosome assembly |
| | | | | | | protein 1-like 1 |
| 197 | NP1L4_HUMAN | Q99733 | GVPSDSVEAAK | 9 | 1 | Nucleosome assembly |
| | | | | | | protein 1-like 4 |
| 198 | NP1L4_HUMAN | Q99733 | GVPSDSVEAAKN | 9 | 1 | Nucleosome assembly |
| | | | ASNTEK | | | protein 1-like 4 |
| | NP1L4_HUMAN | Q99733 | | 9 | 1 | Nucleosome assembly |
| | | | | | | protein 1-like 4 |
| 199 | NP1L4_HUMAN | Q99733 | GVPSDSVEAAKN | 9 | 1 | Nucleosome assembly |
| | | | ASNTEKLTDQVM | | | protein 1-like 4 |
| | | | QNPR | | | |
| 200 | NP1L4_HUMAN | Q99733 | NVPHTPSSYIETLPK | 47 | 1 | Nucleosome assembly |
| | | | | | | protein 1-like 4 |
| | NP60_HUMAN | Q49A26 | | 256 | 1 | Nuclear protein NP60 |
| | NPAT_HUMAN | Q14207 | | 734 | 1 | Protein NPAT |
| | NPM_HUMAN | P06748 | | 7 | 1 | Nucleophosmin |
| | NPM_HUMAN | P06748 | | 4 | 1 | Nucleophosmin |
| | NS1BP_HUMAN | Q9Y6Y0 | | 239 | 1 | Influenza virus NS1A- |
| | | | | | | binding protein |
| | NSBP1_HUMAN | P82970 | | 58 | 1 | Nucleosome-binding protein 1 |
| 201 | NSUN2_HUMAN | Q08J23 | GQKVEVPQPLSW | 109 | 1 | tRNA (cytosine-5-)- |
| | | | YPEELAWHTNLSR | | | methyltransferase NSUN2 |
| 202 | NSUN2_HUMAN | Q08J23 | GQKVEVPQPLSW | 109 | 1 | tRNA (cytosine-5-)- |
| | | | YPEELAWHTNLS | | | methyltransferase NSUN2 |
| | | | RK | | | |
| | NSUN2_HUMAN | Q08J23 | | 500 | 1 | tRNA (cytosine-5-)- |
| | | | | | | methyltransferase NSUN2 |
| | NSUN2_HUMAN | Q08J23 | | 500 | 1 | tRNA (cytosine-5-)- |
| | | | | | | methyltransferase NSUN2 |
| | NSUN2_HUMAN | Q08J23 | | 665 | 1 | tRNA (cytosine-5-)- |
| | | | | | | methyltransferase NSUN2 |
| | NU153_HUMAN | P49790 | | 359 | 1 | Nuclear pore complex |
| | | | | | | protein Nup153 |
| | NUCB2_HUMAN | P80303 | | 259 | 1 | Nucleobindin-2 |
| | NUCB2_HUMAN | P80303 | | 238 | 1 | Nucleobindin-2 |
| | NUCKS_HUMAN | Q9H1E3 | | 30 | 3 | Nuclear ubiquitous casein |
| | | | | | | and cyclin-dependent kinases |
| | | | | | | substrate |
| | NUCL_HUMAN | P19338 | | 637 | 1 | Nucleolin |
| | NUDC3_HUMAN | Q8IVD9 | | 126 | 1 | NudC domain-containing |
| | | | | | | protein 3 |
| | NUDC3_HUMAN | Q8IVD9 | | 126 | 1 | NudC domain-containing |
| | | | | | | protein 3 |
| | NUDC3_HUMAN | Q8IVD9 | | 120 | 1 | NudC domain-containing |
| | | | | | | protein 3 |
| | NUFP2_HUMAN | Q7Z417 | | 452 | 1 | Nuclear fragile X mental |
| | | | | | | retardation-interacting |
| | | | | | | protein 2 |
| | NUMA1_HUMAN | Q14980 | | 1748 | 1 | Nuclear mitotic apparatus |
| | | | | | | protein 1 |
| | NUMA1_HUMAN | Q14980 | | 1748 | 1 | Nuclear mitotic apparatus |
| | | | | | | protein 1 |
| | NUMA1_HUMAN | Q14980 | | 1830 | 1 | Nuclear mitotic apparatus |
| | | | | | | protein 1 |
| 203 | NUP43_HUMAN | Q8NFH3 | GGFEGDHQLLCDIR | 59 | 1 | Nucleoporin Nup43 |
| | NUP50_HUMAN | Q9UKX7 | | 127 | 1 | Nucleoporin 50 kDa |
| 204 | NUP93_HUMAN | Q8N1F7 | FTQESEPSYISDV | 158 | 1 | Nuclear pore complex |
| | | | GPPGR | | | protein Nup93 |
| 205 | ODPB_HUMAN | P11177 | AINQGMDEELER | 38 | 1 | Pyruvate dehydrogenase E1 |
| | | | DEK | | | component subunit beta, |
| | | | | | | mitochondrial |
| | OFD1_HUMAN | O75665 | | 854 | 1 | Oral-facial-digital syndrome |
| | | | | | | 1 protein |
| | ORAV1_HUMAN | Q8WV07 | | 10 | 1 | Oral cancer overexpressed |
| | | | | | | protein 1 |
| | OSBL8_HUMAN | Q9BZF1 | | 807 | 1 | Oxysterol-binding protein- |
| | | | | | | related protein 8 |
| | OTU6B_HUMAN | Q8N6M0 | | 81 | 1 | OTU domain-containing |
| | | | | | | protein 6B |
| | OTUD4_HUMAN | Q01804 | | 10 | 1 | OTU domain-containing |
| | | | | | | protein 4 |
| | OXR1_HUMAN | Q8N573 | | 450 | 1 | Oxidation resistance protein 1 |
| | OXR1_HUMAN | Q8N573 | | 450 | 1 | Oxidation resistance protein 1 |
| | P4R3A_HUMAN | Q6IN85 | | 693 | 1 | Serine/threonine-protein |
| | | | | | | phosphatase 4 regulatory |
| | | | | | | subunit 3A |
| | P66B_HUMAN | Q8WXI9 | | 345 | 1 | Transcriptional repressor |
| | | | | | | p66-beta |
| 206 | PA24A_HUMAN | P47712 | AAVADPDEFER | 523 | 1 | Cytosolic phospholipase A2 |
| 207 | PABP2_HUMAN | Q86U42 | GAIEDPELEAIK | 112 | 1 | Polyadenylate-binding |
| | | | | | | protein 2 |
| 208 | PABP2_HUMAN | Q86U42 | GAIEDPELEAIKAR | 112 | 1 | Polyadenylate-binding |
| | | | | | | protein 2 |
| | PAIRB_HUMAN | Q8NC51 | | 338 | 1 | Plasminogen activator |
| | | | | | | inhibitor 1 RNA-binding |
| | | | | | | protein |
| | PAK1_HUMAN | Q13153 | | 91 | 2 | Serine/threonine-protein |
| | | | | | | kinase PAK 1 |
| | PAK2_HUMAN | Q13177 | | 90 | | Serine/threonine-protein |
| | | | | | | kinase PAK 2 |
| | PAK2_HUMAN | Q13177 | | 149 | 1 | Serine/threonine-protein |
| | | | | | | kinase PAK 2 |
| | PALLD_HUMAN | Q8WX93 | | 433 | 1 | Palladin |
| | PARG_HUMAN | Q86W56 | | 257 | 1 | Poly(ADP-ribose) |
| | | | | | | glycohydrolase |
| | PARP1_HUMAN | P09874 | | 215 | 1 | Poly [ADP-ribose] |
| | | | | | | polymerase 1 |
| | PARP1_HUMAN | P09874 | | 215 | 1 | Poly [ADP-ribose] |
| | | | | | | polymerase 1 |
| | PARP1_HUMAN | P09874 | | 215 | 1 | Poly [ADP-ribose] |
| | | | | | | polymerase 1 |
| | PARP1_HUMAN | P09874 | | 73 | 1 | Poly [ADP-ribose] |
| | | | | | | polymerase 1 |
| | PAWR_HUMAN | Q96IZ0 | | 132 | 1 | PRKC apoptosis WT1 |
| | | | | | | regulator protein |
| | PAXI_HUMAN | P49023 | | 103 | 1 | Paxillin |
| | PAXI_HUMAN | P49023 | | 6 | 1 | Paxillin |
| | PAXI_HUMAN | P49023 | | 336 | 1 | Paxillin |
| | PB1_HUMAN | Q86U86 | | 22 | 1 | Protein polybromo-1 |
| | PCBP1_HUMAN | Q15365 | | 204 | 1 | Poly(rC)-binding protein 1 |
| 209 | PCBP1_HUMAN | Q15365 | AYSIQGQHTISPL | 221 | 1 | Poly(rC)-binding protein 1 |
| | | | DLAK | | | |
| 210 | PCBP1_HUMAN | Q15365 | ASTQTTHELTIPN | 276 | 1 | Poly(rC)-binding protein 1 |
| | | | NLIGCIIGR | | | |
| | PCBP2_HUMAN | Q15366 | | 283 | 1 | Poly(rC)-binding protein 2 |
| | PCF11_HUMAN | O94913 | | 1289 | 1 | Pre-mRNA cleavage |
| | | | | | | complex 2 protein Pcf11 |
| 211 | PCM1_HUMAN | Q15154 | GRGEPAMESSQIV | 194 | 1 | Pericentriolar material 1 |
| | | | SR | | | protein |
| | PCM1_HUMAN | Q15154 | | 1552 | 1 | Pericentriolar material 1 |
| | | | | | | protein |
| | PCNT_HUMAN | O95613 | | 81 | 1 | Pericentrin |
| 212 | PDIP3_HUMAN | Q9BY77 | AYTAPALPSSIR | 235 | 1 | Polymerase delta-interacting |
| | | | | | | protein 3 |
| | PDLI1_HUMAN | O00151 | | 55 | 1 | PDZ and LIM domain |
| | | | | | | protein 1 |
| | PDXD1_HUMAN | Q6P996 | | 585 | 1 | Pyridoxal-dependent |
| | | | | | | decarboxylase domain- |
| | | | | | | containing protein 1 |
| | PEBB_HUMAN | Q13951 | | 121 | 1 | Core-binding factor subunit |
| | | | | | | beta |
| | PFTK1_HUMAN | O94921 | | 57 | 1 | Serine/threonine-protein |
| | | | | | | kinase PFTAIRE-1 |
| 213 | PGK1_HUMAN | P00558 | CVGPEVEK | 99 | 1 | Phosphoglycerate kinase 1 |
| 214 | PGK1_HUMAN | P00558 | CVGPEVEKACAN | 99 | 1 | Phosphoglycerate kinase 1 |
| | | | PAAGSVILLENLR | | | |
| | PGK1_HUMAN | P00558 | | 286 | 1 | Phosphoglycerate kinase 1 |
| | PGK1_HUMAN | P00558 | | 69 | 1 | Phosphoglycerate kinase 1 |
| | PGK1_HUMAN | P00558 | | 160 | 2 | Phosphoglycerate kinase 1 |
| | PGK2_HUMAN | P07205 | | 160 | | Phosphoglycerate kinase 2 |
| | PHAR4_HUMAN | Q8IZ21 | | 21 | 1 | Phosphatase and actin |
| | | | | | | regulator 4 |
| | PHF3_HUMAN | Q92576 | | 1627 | 1 | PHD finger protein 3 |
| | PHF3_HUMAN | Q92576 | | 1100 | 1 | PHD finger protein 3 |
| | PHF3_HUMAN | Q92576 | | 1158 | 1 | PHD finger protein 3 |
| | PHF3_HUMAN | Q92576 | | 1398 | 1 | PHD finger protein 3 |
| | PHTNS_HUMAN | Q6NYC8 | | 496 | 1 | Phostensin |
| 215 | PI4KB_HUMAN | Q9UBF8 | SITSQESKEPVFIA | 489 | 1 | Phosphatidylinositol 4-kinase |
| | | | AGDIR | | | beta |
| 216 | PI4KB_HUMAN | Q9UBF8 | SITSQESKEPVFIA | 489 | 1 | Phosphatidylinositol 4-kinase |
| | | | AGDIRR | | | beta |
| 217 | PIAS1_HUMAN | O75925 | GHPASSPLLPVSL | 101 | 1 | E3 SUMO-protein ligase |
| | | | LGPK | | | PIAS1 |
| | PICAL_HUMAN | Q13492 | | 277 | 1 | Phosphatidylinositol-binding |
| | | | | | | clathrin assembly protein |
| | PITM1_HUMAN | O00562 | | 379 | 1 | Membrane-associated |
| | | | | | | phosphatidylinositol transfer |
| | | | | | | protein 1 |
| | PJA2_HUMAN | O43164 | | 87 | 1 | E3 ubiquitin-protein ligase |
| | | | | | | Praja2 |
| | PKHG1_HUMAN | Q9ULL1 | | 436 | 1 | Pleckstrin homology |
| | | | | | | domain-containing family G |
| | | | | | | member 1 |
| | PKP4_HUMAN | Q99569 | | 804 | 1 | Plakophilin-4 |
| | PLCG1_HUMAN | P19174 | | 771 | 1 | 1-phosphatidylinositol-4,5- |
| | | | | | | bisphosphate |
| | | | | | | phosphodiesterase gamma-1 |
| | PLDN_HUMAN | Q9UL45 | | 11 | 1 | Pallidin |
| | POGZ_HUMAN | Q7Z3K3 | | 28 | 1 | Pogo transposable element |
| | | | | | | with ZNF domain |
| | POMP_HUMAN | Q9Y244 | | 13 | 1 | Proteasome maturation |
| | | | | | | protein |
| | PP1RA_HUMAN | Q96QC0 | | 377 | 1 | Serine/threonine-protein |
| | | | | | | phosphatase 1 regulatory |
| | | | | | | subunit 10 |
| | PP1RA_HUMAN | Q96QC0 | | 294 | 1 | Serine/threonine-protein |
| | | | | | | phosphatase 1 regulatory |
| | | | | | | subunit 10 |
| | PP1RA_HUMAN | Q96QC0 | | 367 | 1 | Serine/threonine-protein |
| | | | | | | phosphatase 1 regulatory |
| | | | | | | subunit 10 |
| | PP4R1_HUMAN | Q8TF05 | | 445 | 1 | Serine/threonine-protein |
| | | | | | | phosphatase 4 regulatory |
| | | | | | | subunit 1 |
| | PPIA_HUMAN | P62937 | | 10 | 1 | Peptidyl-prolyl cis-trans |
| | | | | | | isomerase A |
| 218 | PPIL4_HUMAN | Q8WUA2 | ADIKPPENVLFVCK | 233 | 1 | Peptidyl-prolyl cis-trans |
| | | | | | | isomerase-like 4 |
| | PPR3D_HUMAN | O95685 | | 32 | 1 | Protein phosphatase 1 |
| | | | | | | regulatory subunit 3D |
| | PR40A_HUMAN | O75400 | | 134 | 1 | Pre-mRNA-processing factor |
| | | | | | | 40 homolog A |
| | PRD15_HUMAN | P57071 | | 1270 | 1 | PR domain zinc finger |
| | | | | | | protein 15 |
| | PRD15_HUMAN | P57071 | | 1270 | 1 | PR domain zinc finger |
| | | | | | | protein 15 |
| 219 | PRKDC_HUMAN | P78527 | GDPSDRMEVQEQ | 3212 | 1 | DNA-dependent protein |
| | | | EEDISSLIR | | | kinase catalytic subunit |
| | PROF1_HUMAN | P07737 | | 20 | 1 | Profilin-1 |
| | PROF1_HUMAN | P07737 | | 82 | 1 | Profilin-1 |
| | PROF1_HUMAN | P07737 | | 82 | 1 | Profilin-1 |
| | PROF1_HUMAN | P07737 | | 15 | 1 | Profilin-1 |
| | PRP17_HUMAN | O60508 | | 56 | 1 | Pre-mRNA-processing factor |
| | | | | | | 17 |
| 220 | PRP17_HUMAN | O60508 | VAKPSEEEQKELD | 205 | 1 | Pre-mRNA-processing factor |
| | | | EITAKR | | | 17 |
| | PRP17_HUMAN | O60508 | | 191 | 1 | Pre-mRNA-processing factor |
| | | | | | | 17 |
| | PRP17_HUMAN | O60508 | | 205 | 1 | Pre-mRNA-processing factor |
| | | | | | | 17 |
| | PRP17_HUMAN | O60508 | | 205 | 1 | Pre-mRNA-processing factor |
| | | | | | | 17 |
| | PRP31_HUMAN | Q8WWY3 | | 387 | 1 | U4/U6 small nuclear |
| | | | | | | ribonucleoprotein Prp31 |
| | PRR12_HUMAN | Q9ULL5 | | 116 | 1 | Proline-rich protein 12 |
| | PRR3_HUMAN | P79522 | | 32 | 1 | Proline-rich protein 3 |
| | PRS10_HUMAN | P62333 | | 266 | 1 | 26S protease regulatory |
| | | | | | | subunit S10B |
| 221 | PRS6A_HUMAN | P17980 | GIGEEVLK | 28 | 1 | 26S protease regulatory |
| | | | | | | subunit 6A |
| | PRS6A_HUMAN | P17980 | | 28 | 1 | 26S protease regulatory |
| | | | | | | subunit 6A |
| 222 | PRS6A_HUMAN | P17980 | GIGEEVLKMSTEE | 28 | 1 | 26S protease regulatory |
| | | | IIQR | | | subunit 6A |
| | PRS6A_HUMAN | P17980 | | 319 | 1 | 26S protease regulatory |
| | | | | | | subunit 6A |
| 223 | PRS6B_HUMAN | P43686 | GFDQNVNVK | 298 | 1 | 26S protease regulatory |
| | | | | | | subunit 6B |
| 224 | PRS8_HUMAN | P62195 | SIGSSRLEGGSGG | 253 | 1 | 26S protease regulatory |
| | | | DSEVQR | | | subunit 8 |
| | PSA5_HUMAN | P28066 | | 72 | 1 | Proteasome subunit alpha |
| | | | | | | type-5 |
| | PSA7L_HUMAN | Q8TAA3 | | 16 | 2 | Proteasome subunit alpha |
| | | | | | | type-7-like |
| | PSA7_HUMAN | O14818 | | 14 | | Proteasome subunit alpha |
| | | | | | | type-7 |
| | PSB1_HUMAN | P20618 | | 48 | 1 | Proteasome subunit beta |
| | | | | | | type-1 |
| 225 | PSB4_HUMAN | P28070 | SFMDPASALYR | 30 | 1 | Proteasome subunit beta |
| | | | | | | type-4 |
| | PSB7_HUMAN | Q99436 | | 54 | 1 | Proteasome subunit beta |
| | | | | | | type-7 |
| 226 | PSD12_HUMAN | O00232 | YSATVDQR | 20 | 1 | 26S proteasome non-ATPase |
| | | | | | | regulatory subunit 12 |
| | PSD4_HUMAN | Q8NDX1 | | 83 | 1 | PH and SEC7 domain- |
| | | | | | | containing protein 4 |
| | PSD4_HUMAN | Q8NDX1 | | 536 | 1 | PH and SEC7 domain- |
| | | | | | | containing protein 4 |
| | PSIP1_HUMAN | O75475 | | 31 | 1 | PC4 and SFRS1-interacting |
| | | | | | | protein |
| | PSIP1_HUMAN | O75475 | | 31 | 1 | PC4 and SFRS1-interacting |
| | | | | | | protein |
| 227 | PSIP1_HUMAN | O75475 | SVITQVLNK | 434 | 1 | PC4 and SFRS1-interacting |
| | | | | | | protein |
| | PSME3_HUMAN | P61289 | | 78 | 1 | Proteasome activator |
| | | | | | | complex subunit 3 |
| | PTBP1_HUMAN | P26599 | | 3 | 1 | Polypyrimidine tract-binding |
| | | | | | | protein 1 |
| | PTBP1_HUMAN | P26599 | | 3 | 1 | Polypyrimidine tract-binding |
| | | | | | | protein 1 |
| | PTBP1_HUMAN | P26599 | | 173 | 1 | Polypyrimidine tract-binding |
| | | | | | | protein 1 |
| | PTBP1_HUMAN | P26599 | | 173 | 1 | Polypyrimidine tract-binding |
| | | | | | | protein 1 |
| | PTBP1_HUMAN | P26599 | | 173 | 1 | Polypyrimidine tract-binding |
| | | | | | | protein 1 |
| | PTBP1_HUMAN | P26599 | | 173 | 1 | Polypyrimidine tract-binding |
| | | | | | | protein 1 |
| | PTBP1_HUMAN | P26599 | | 140 | 2 | Polypyrimidine tract-binding |
| | | | | | | protein 1 |
| | PTCA_HUMAN | Q14761 | | 121 | 1 | Protein tyrosine phosphatase |
| | | | | | | receptor type C-associated |
| | | | | | | protein |
| | PTCA_HUMAN | Q14761 | | 121 | 1 | Protein tyrosine phosphatase |
| | | | | | | receptor type C-associated |
| | | | | | | protein |
| | PTCA_HUMAN | Q14761 | | 117 | 1 | Protein tyrosine phosphatase |
| | | | | | | receptor type C-associated |
| | | | | | | protein |
| | PTMA_HUMAN | P06454 | | 4 | 1 | Prothymosin alpha |
| | PTMA_HUMAN | P06454 | | 4 | 1 | Prothymosin alpha |
| | PTMA_HUMAN | P06454 | | 8 | 1 | Prothymosin alpha |
| | PTMA_HUMAN | P06454 | | 8 | 1 | Prothymosin alpha |
| 228 | PTN3_HUMAN | P26045 | GVDQQLLDDFHR | 472 | 1 | Tyrosine-protein phosphatase |
| | | | | | | non-receptor type 3 |
| 229 | PUR2_HUMAN | P22102 | GGPNTGGMGAYC | 226 | 1 | Trifunctional purine |
| | | | PAPQVSNDLLLK | | | biosynthetic protein |
| | | | | | | adenosine-3 |
| | PUR2_HUMAN | P22102 | | 206 | 1 | Trifunctional purine |
| | | | | | | biosynthetic protein |
| | | | | | | adenosine-3 |
| | PUR2_HUMAN | P22102 | | 444 | 1 | Trifunctional purine |
| | | | | | | biosynthetic protein |
| | | | | | | adenosine-3 |
| | PUR6_HUMAN | P22234 | | 320 | 1 | Multifunctional protein |
| | | | | | | ADE2 |
| | PUR6_HUMAN | P22234 | | 27 | 1 | Multifunctional protein |
| | | | | | | ADE2 |
| 230 | PUR9_HUMAN | P31939 | GIIAPGYEEEALTI | 340 | 1 | Bifunctional purine |
| | | | LSK | | | biosynthesis protein PURH |
| | PUS7_HUMAN | Q96PZ0 | | 51 | 1 | Pseudouridylate synthase 7 |
| | | | | | | homolog |
| | PUS7_HUMAN | Q96PZ0 | | 23 | 1 | Pseudouridylate synthase 7 |
| | | | | | | homolog |
| 231 | PWP2A_HUMAN | Q96N64 | GQQSAPQADEPPL | 56 | 1 | PWWP domain-containing |
| | | | PPPPPPPGELAR | | | protein 2A |
| | PYR1_HUMAN | P27708 | | 1139 | 1 | CAD protein |
| | QKI_HUMAN | Q96PU8 | | 75 | 1 | Protein quaking |
| | QN1_HUMAN | Q5TB80 | | 248 | 1 | Protein QN1 homolog |
| | QSER1_HUMAN | Q2KHR3 | | 1322 | 1 | Glutamine and serine-rich |
| | | | | | | protein 1 |
| 232 | QSK_HUMAN | Q9Y2K2 | GTLNLDSDEGEEP | 384 | 1 | Serine/threonine-protein |
| | | | SPEALVR | | | kinase QSK |
| | R3HD1_HUMAN | Q15032 | | 500 | 1 | R3H domain-containing |
| | | | | | | protein 1 |
| | R3HD1_HUMAN | Q15032 | | 500 | 1 | R3H domain-containing |
| | | | | | | protein 1 |
| | RA1L3_HUMAN | P0C7M2 | | 158 | 3 | Putative heterogeneous |
| | | | | | | nuclear ribonucleoprotein |
| | | | | | | A1-like protein 3 |
| | ROA1L_HUMAN | Q32P51 | | 158 | | Heterogeneous nuclear |
| | | | | | | ribonucleoprotein A1-like |
| | | | | | | protein |
| | ROA1_HUMAN | P09651 | | 158 | | Heterogeneous nuclear |
| | | | | | | ribonucleoprotein A1 |
| | RA1L3_HUMAN | P0C7M2 | | 95 | 3 | Putative heterogeneous |
| | | | | | | nuclear ribonucleoprotein |
| | | | | | | A1-like protein 3 |
| | ROA1L_HUMAN | Q32P51 | | 95 | | Heterogeneous nuclear |
| | | | | | | ribonucleoprotein A1-like |
| | | | | | | protein |
| | ROA1_HUMAN | P09651 | | 95 | | Heterogeneous nuclear |
| | | | | | | ribonucleoprotein A1 |
| | RA1L3_HUMAN | P0C7M2 | | 70 | 2 | Putative heterogeneous |
| | | | | | | nuclear ribonucleoprotein |
| | | | | | | A1-like protein 3 |
| | ROA1_HUMAN | P09651 | | 70 | | Heterogeneous nuclear |
| | | | | | | ribonucleoprotein A1 |
| | RA1L3_HUMAN | P0C7M2 | | 158 | 2 | Putative heterogeneous |
| | | | | | | nuclear ribonucleoprotein |
| | | | | | | A1-like protein 3 |
| | ROA1_HUMAN | P09651 | | 158 | | Heterogeneous nuclear |
| | | | | | | ribonucleoprotein A1 |
| | RA1L3_HUMAN | P0C7M2 | | 70 | 2 | Putative heterogeneous |
| | | | | | | nuclear ribonucleoprotein |
| | | | | | | A1-like protein 3 |
| | ROA1_HUMAN | P09651 | | 70 | | Heterogeneous nuclear |
| | | | | | | ribonucleoprotein A1 |
| 233 | RAD21_HUMAN | O60216 | SVDPVEPMPTMT | 280 | 1 | Double-strand-break repair |
| | | | DQTTLVPNEEEAF | | | protein rad21 homolog |
| | | | ALEPIDITVK | | | |
| | RAD21_HUMAN | O60216 | | 280 | 1 | Double-strand-break repair |
| | | | | | | protein rad21 homolog |
| 234 | RAD21_HUMAN | O60216 | SVDPVEPMPTMT | 280 | 1 | Double-strand-break repair |
| | | | DQTTLVPNEEEAF | | | protein rad21 homolog |
| | | | ALEPIDITVKETK | | | |
| 235 | RAD21_HUMAN | O60216 | VAQQFSLNQSR | 129 | 1 | Double-strand-break repair |
| | | | | | | protein rad21 homolog |
| | RADIL_HUMAN | Q96JH8 | | 842 | 1 | Ras-associating and dilute |
| | | | | | | domain-containing protein |
| | RADIL_HUMAN | Q96JH8 | | 842 | 1 | Ras-associating and dilute |
| | | | | | | domain-containing protein |
| | RANG_HUMAN | P43487 | | 128 | 1 | Ran-specific GTPase- |
| | | | | | | activating protein |
| | RB3GP_HUMAN | Q15042 | | 253 | 1 | Rab3 GTPase-activating |
| | | | | | | protein catalytic subunit |
| | RBBP4_HUMAN | Q09028 | | 362 | 2 | Histone-binding protein |
| | | | | | | RBBP4 |
| | RBBP7_HUMAN | Q16576 | | 361 | | Histone-binding protein |
| | | | | | | RBBP7 |
| | RBBP6_HUMAN | Q7Z6E9 | | 973 | 1 | Retinoblastoma-binding |
| | | | | | | protein 6 |
| | RBBP6_HUMAN | Q7Z6E9 | | 1679 | 1 | Retinoblastoma-binding |
| | | | | | | protein 6 |
| | RBBP6_HUMAN | Q7Z6E9 | | 1268 | 1 | Retinoblastoma-binding |
| | | | | | | protein 6 |
| | RBBP6_HUMAN | Q7Z6E9 | | 1268 | 1 | Retinoblastoma-binding |
| | | | | | | protein 6 |
| 236 | RBBP7_HUMAN | Q16576 | SDKGEFGGFGSVT | 99 | 1 | Histone-binding protein |
| | | | GK | | | RBBP7 |
| | RBBP7_HUMAN | Q16576 | | 94 | 1 | Histone-binding protein |
| | | | | | | RBBP7 |
| | RBBP8_HUMAN | Q99708 | | 743 | 1 | Retinoblastoma-binding |
| | | | | | | protein 8 |
| | RBM15_HUMAN | Q96T37 | | 751 | 1 | Putative RNA-binding |
| | | | | | | protein 15 |
| 237 | RBM16_HUMAN | Q9UPN6 | GVEEEVFEQEAK | 381 | 1 | Putative RNA-binding |
| | | | | | | protein 16 |
| | RBM16_HUMAN | Q9UPN6 | | 776 | 1 | Putative RNA-binding |
| | | | | | | protein 16 |
| | RBM25_HUMAN | P49756 | | 634 | 1 | Probable RNA-binding |
| | | | | | | protein 25 |
| | RBM26_HUMAN | Q5T8P6 | | 432 | 1 | RNA-binding protein 26 |
| 238 | RBM26_HUMAN | Q5T8P6 | GYNPEAPSITNTS | 432 | 1 | RNA-binding protein 26 |
| | | | RPMYR | | | |
| | RBM26_HUMAN | Q5T8P6 | | 281 | 1 | RNA-binding protein 26 |
| | RBM26_HUMAN | Q5T8P6 | | 281 | 1 | RNA-binding protein 26 |
| | RBM27_HUMAN | Q9P2N5 | | 488 | 1 | RNA-binding protein 27 |
| | RBM28_HUMAN | Q9NW13 | | 245 | 1 | RNA-binding protein 28 |
| | RBM33_HUMAN | Q96EV2 | | 999 | 1 | RNA-binding protein 33 |
| 239 | RBM39_HUMAN | Q14498 | ASSASSFLDSDEL | 332 | 1 | RNA-binding protein 39 |
| | | | ER | | | |
| 240 | RBM39_HUMAN | Q14498 | ASSASSFLDSDEL | 332 | 1 | RNA-binding protein 39 |
| | | | ERTGIDLGTTGR | | | |
| | RBM8A_HUMAN | Q9Y5S9 | | 7 | 1 | RNA-binding protein 8A |
| | RBM8A_HUMAN | Q9Y5S9 | | 7 | 1 | RNA-binding protein 8A |
| | RBM8A_HUMAN | Q9Y5S9 | | 7 | 1 | RNA-binding protein 8A |
| | RBM8A_HUMAN | Q9Y5S9 | | 7 | 1 | RNA-binding protein 8A |
| | RBM8A_HUMAN | Q9Y5S9 | | 7 | 1 | RNA-binding protein 8A |
| 241 | RBM8A_HUMAN | Q9Y5S9 | SVEQDGDEPGPQR | 56 | 1 | RNA-binding protein 8A |
| | RBM9_HUMAN | O43251 | | 103 | 1 | RNA-binding protein 9 |
| | RBP2_HUMAN | P49792 | | 2491 | 1 | E3 SUMO-protein ligase |
| | | | | | | RanBP2 |
| 242 | RBP2_HUMAN | P49792 | GGSAHGDDDDDG | 1158 | 1 | E3 SUMO-protein ligase |
| | | | PHFEPVVPLPDKI | | | RanBP2 |
| | | | EVK | | | |
| 243 | RBP2_HUMAN | P49792 | GTGGQSIYGDKFE | 3132 | 1 | E3 SUMO-protein ligase |
| | | | DENFDVK | | | RanBP2 |
| | RBP2_HUMAN | P49792 | | 2861 | 1 | E3 SUMO-protein ligase |
| | | | | | | RanBP2 |
| | RBP2_HUMAN | P49792 | | 1158 | 1 | E3 SUMO-protein ligase |
| | | | | | | RanBP2 |
| | RBP2_HUMAN | P49792 | | 2307 | 7 | E3 SUMO-protein ligase |
| | | | | | | RanBP2 |
| | RGPD1_HUMAN | Q68DN6 | | 1316 | | RANBP2-like and GRIP |
| | | | | | | domain-containing protein 1 |
| | RGPD3_HUMAN | A6NKT7 | | 1332 | | RANBP2-like and GRIP |
| | | | | | | domain-containing protein 3 |
| | RGPD4_HUMAN | Q7Z3J3 | | 1332 | | RANBP2-like and GRIP |
| | | | | | | domain-containing protein 4 |
| | RGPD5_HUMAN | Q99666 | | 1331 | | RANBP2-like and GRIP |
| | | | | | | domain-containing protein 5 |
| | RGPD6_HUMAN | Q53T03 | | 1331 | | RANBP2-like and GRIP |
| | | | | | | domain-containing protein 6 |
| | RGPD8_HUMAN | O14715 | | 321 | | RANBP2-like and GRIP |
| | | | | | | domain-containing protein 8 |
| | | | | | | (Fragment) |
| | RBP2_HUMAN | P49792 | | 2237 | 6 | E3 SUMO-protein ligase |
| | | | | | | RanBP2 |
| | RGPD3_HUMAN | A6NKT7 | | 1262 | | RANBP2-like and GRIP |
| | | | | | | domain-containing protein 3 |
| | RGPD4_HUMAN | Q7Z3J3 | | 1262 | | RANBP2-like and GRIP |
| | | | | | | domain-containing protein 4 |
| | RGPD5_HUMAN | Q99666 | | 1261 | | RANBP2-like and GRIP |
| | | | | | | domain-containing protein 5 |
| | RGPD6_HUMAN | Q53T03 | | 1261 | | RANBP2-like and GRIP |
| | | | | | | domain-containing protein 6 |
| | RGPD8_HUMAN | O14715 | | 251 | | RANBP2-like and GRIP |
| | | | | | | domain-containing protein 8 |
| | | | | | | (Fragment) |
| | RBP56_HUMAN | Q92804 | | 141 | 1 | TATA-binding protein- |
| | | | | | | associated factor 2N |
| | RBTN1_HUMAN | P25800 | | 9 | 1 | Rhombotin-1 |
| | RBY1B_HUMAN | A6NDE4 | | 467 | 3 | RNA-binding motif protein, |
| | | | | | | Y chromosome, family 1 |
| | | | | | | member B |
| | RBY1F_HUMAN | Q15415 | | 467 | | RNA-binding motif protein, |
| | | | | | | Y chromosome, family 1 |
| | | | | | | member F/J |
| | RBY1H_HUMAN | Q15378 | | 327 | | Putative RNA-binding motif |
| | | | | | | protein, Y chromosome, |
| | | | | | | family 1 member H |
| 244 | RB_HUMAN | P06400 | SIDSFETQR | 347 | 1 | Retinoblastoma-associated |
| | | | | | | protein |
| | RCAN1_HUMAN | P53805 | | 4 | 1 | Calcipressin-1 |
| | RCC2_HUMAN | Q9P258 | | 61 | 1 | Protein RCC2 |
| | RCC2_HUMAN | Q9P258 | | 61 | 1 | Protein RCC2 |
| | RCN2_HUMAN | Q14257 | | 204 | 1 | Reticulocalbin-2 |
| | RCOR2_HUMAN | Q8IZ40 | | 392 | 1 | REST corepressor 2 |
| | RCOR2_HUMAN | Q8IZ40 | | 392 | 1 | REST corepressor 2 |
| | RCOR2_HUMAN | Q8IZ40 | | 392 | 1 | REST corepressor 2 |
| | RCOR2_HUMAN | Q8IZ40 | | 392 | 1 | REST corepressor 2 |
| | RD23B_HUMAN | P54727 | | 166 | 1 | UV excision repair protein |
| | | | | | | RAD23 homolog B |
| 245 | RED_HUMAN | Q13123 | GVNKDYEETELIS | 109 | 1 | Protein Red |
| | | | TTANYR | | | |
| 246 | RED_HUMAN | Q13123 | YVPSTTK | 325 | 2 | Protein Red |
| 247 | RED_HUMAN | Q13123 | YVPSTTKTPR | 325 | 1 | Protein Red |
| 248 | REL_HUMAN | Q04864 | GYYEAEFGQER | 87 | 1 | C-Rel proto-oncogene |
| | | | | | | protein |
| | RENT1_HUMAN | Q92900 | | 76 | 1 | Regulator of nonsense |
| | | | | | | transcripts 1 |
| 249 | REPS1_HUMAN | Q96D71 | SFTSDPEQIGSNV | 466 | 1 | RalBP1-associated Eps |
| | | | TR | | | domain-containing protein 1 |
| 250 | REPS1_HUMAN | Q96D71 | SNIAPADPDTAIV | 387 | 1 | RalBP1-associated Eps |
| | | | HPVPIR | | | domain-containing protein 1 |
| 251 | REPS1_HUMAN | Q96D71 | GYSSSDSFTSDPE | 460 | 1 | RalBP1-associated Eps |
| | | | QIGSNVTR | | | domain-containing protein 1 |
| | REQU_HUMAN | Q92785 | | 244 | 1 | Zinc finger protein ubi-d4 |
| 252 | REQU_HUMAN | Q92785 | GSSLEALLR | 116 | 1 | Zinc finger protein ubi-d4 |
| | REST_HUMAN | Q13127 | | 942 | 1 | RE1-silencing transcription |
| | | | | | | factor |
| | RFC1_HUMAN | P35251 | | 724 | 1 | Replication factor C subunit 1 |
| 253 | RFC1_HUMAN | P35251 | GMAGNEDR | 724 | 1 | Replication factor C subunit 1 |
| 254 | RFC1_HUMAN | P35251 | GMAGNEDRGGIQ | 724 | 1 | Replication factor C subunit 1 |
| | | | ELIGLIK | | | |
| | RFC1_HUMAN | P35251 | | 168 | 1 | Replication factor C subunit 1 |
| | RFX7_HUMAN | Q2KHR2 | | 480 | 1 | DNA-binding protein RFX7 |
| | RGAP1_HUMAN | Q9H0H5 | | 274 | 1 | Rac GTPase-activating |
| | | | | | | protein 1 |
| | RGAP1_HUMAN | Q9H0H5 | | 274 | 1 | Rac GTPase-activating |
| | | | | | | protein 1 |
| | RGPD1_HUMAN | Q68DN6 | | 1500 | 7 | RANBP2-like and GRIP |
| | | | | | | domain-containing protein 1 |
| | RGPD2_HUMAN | P0C839 | | 765 | | RANBP2-like and GRIP |
| | | | | | | domain-containing protein 2 |
| | RGPD3_HUMAN | A6NKT7 | | 1516 | | RANBP2-like and GRIP |
| | | | | | | domain-containing protein 3 |
| | RGPD4_HUMAN | Q7Z3J3 | | 1516 | | RANBP2-like and GRIP |
| | | | | | | domain-containing protein 4 |
| | RGPD5_HUMAN | Q99666 | | 1515 | | RANBP2-like and GRIP |
| | | | | | | domain-containing protein 5 |
| | RGPD6_HUMAN | Q53T03 | | 1515 | | RANBP2-like and GRIP |
| | | | | | | domain-containing protein 6 |
| | RGPD8_HUMAN | O14715 | | 505 | | RANBP2-like and GRIP |
| | | | | | | domain-containing protein 8 |
| | | | | | | (Fragment) |
| | RGS10_HUMAN | O43665 | | 15 | 1 | Regulator of G-protein |
| | | | | | | signaling 10 |
| | RGS10_HUMAN | O43665 | | 13 | 1 | Regulator of G-protein |
| | | | | | | signaling 10 |
| | RHG04_HUMAN | P98171 | | 404 | 1 | Rho GTPase-activating |
| | | | | | | protein 4 |
| | RHG04_HUMAN | P98171 | | 404 | 1 | Rho GTPase-activating |
| | | | | | | protein 4 |
| 255 | RHG25_HUMAN | P42331 | SFSSMTSDSDTTS | 388 | 1 | Rho GTPase-activating |
| | | | PTGQQPSDAFPED | | | protein 25 |
| | | | SSKVPR | | | |
| | RHG25_HUMAN | P42331 | | 398 | 1 | Rho GTPase-activating |
| | | | | | | protein 25 |
| 256 | RHG30_HUMAN | Q7Z6I6 | GCLCPCSLGLGG | 908 | 1 | Rho GTPase-activating |
| | | | VGMR | | | protein 30 |
| | RHG30_HUMAN | Q7Z6I6 | | 593 | 1 | Rho GTPase-activating |
| | | | | | | protein 30 |
| 257 | RHG30_HUMAN | Q7Z6I6 | SIEAAEGEQEPEA | 364 | 1 | Rho GTPase-activating |
| | | | EALGGTNSEPGTPR | | | protein 30 |
| | RHGBA_HUMAN | Q6P4F7 | | 257 | 1 | Rho GTPase-activating |
| | | | | | | protein 11A |
| 258 | RHOA_HUMAN | P61586 | SLENIPEKWTPEVK | 91 | 2 | Transforming protein RhoA |
| 258 | RHOC_HUMAN | P08134 | SLENIPEKWTPEVK | 91 | | Rho-related GTP-binding |
| | | | | | | protein RhoC |
| | RIF1_HUMAN | Q5UIP0 | | 1810 | 1 | Telomere-associated protein |
| | | | | | | RIF1 |
| | RIF1_HUMAN | Q5UIP0 | | 2001 | 1 | Telomere-associated protein |
| | | | | | | RIF1 |
| | RIMB1_HUMAN | O95153 | | 1808 | 1 | Peripheral-type |
| | | | | | | benzodiazepine receptor- |
| | | | | | | associated protein 1 |
| | RIMB1_HUMAN | O95153 | | 45 | 1 | Peripheral-type |
| | | | | | | benzodiazepine receptor- |
| | | | | | | associated protein 1 |
| 259 | RING1_HUMAN | Q06587 | GTEIAVSPR | 32 | 1 | E3 ubiquitin-protein ligase |
| | | | | | | RING1 |
| | RIOK1_HUMAN | Q9BRS2 | | 130 | 1 | Serine/threonine-protein |
| | | | | | | kinase RIO1 |
| | RIOK1_HUMAN | Q9BRS2 | | 130 | 1 | Serine/threonine-protein |
| | | | | | | kinase RIO1 |
| | RIPK1_HUMAN | Q13546 | | 559 | 1 | Receptor-interacting |
| | | | | | | serine/threonine-protein |
| | | | | | | kinase 1 |
| | RIR2_HUMAN | P31350 | | 30 | 1 | Ribonucleoside-diphosphate |
| | | | | | | reductase subunit M2 |
| | RL17_HUMAN | P18621 | | 111 | 1 | 60S ribosomal protein L17 |
| 260 | RL5_HUMAN | P46777 | GQPGAFTCYLDA | 137 | 1 | 60S ribosomal protein L5 |
| | | | GLAR | | | |
| | RL5_HUMAN | P46777 | | 169 | 1 | 60S ribosomal protein L5 |
| | RN168_HUMAN | Q8IYW5 | | 251 | 1 | RING finger protein 168 |
| 261 | RN213_HUMAN | Q63HN8 | GVREEDLAPFSLR | 356 | 1 | RING finger protein 213 |
| | RN219_HUMAN | Q5W0B1 | | 434 | 1 | RING finger protein 219 |
| | RN220_HUMAN | Q5VTB9 | | 414 | 1 | RING finger protein 220 |
| | RNF5_HUMAN | Q99942 | | 9 | 1 | E3 ubiquitin-protein ligase |
| | | | | | | RNF5 |
| | RNZ1_HUMAN | Q9H777 | | 280 | 1 | Zinc phosphodiesterase |
| | | | | | | ELAC protein 1 |
| | ROA0_HUMAN | Q13151 | | 63 | 1 | Heterogeneous nuclear |
| | | | | | | ribonucleoprotein A0 |
| | ROA0_HUMAN | Q13151 | | 74 | 1 | Heterogeneous nuclear |
| | | | | | | ribonucleoprotein A0 |
| | ROA0_HUMAN | Q13151 | | 63 | 1 | Heterogeneous nuclear |
| | | | | | | ribonucleoprotein A0 |
| | ROA0_HUMAN | Q13151 | | 63 | 1 | Heterogeneous nuclear |
| | | | | | | ribonucleoprotein A0 |
| | ROA2_HUMAN | P22626 | | 77 | 1 | Heterogeneous nuclear |
| | | | | | | ribonucleoproteins A2/B1 |
| | ROA2_HUMAN | P22626 | | 77 | 1 | Heterogeneous nuclear |
| | | | | | | ribonucleoproteins A2/B1 |
| | ROA2_HUMAN | P22626 | | 77 | 1 | Heterogeneous nuclear |
| | | | | | | ribonucleoproteins A2/B1 |
| | ROA2_HUMAN | P22626 | | 131 | 1 | Heterogeneous nuclear |
| | | | | | | ribonucleoproteins A2/B1 |
| | ROA3_HUMAN | P51991 | | 91 | 1 | Heterogeneous nuclear |
| | | | | | | ribonucleoprotein A3 |
| 262 | ROA3_HUMAN | P51991 | SVKPGAHLTVKK | 116 | 1 | Heterogeneous nuclear |
| | | | | | | ribonucleoprotein A3 |
| | ROA3_HUMAN | P51991 | | 179 | 1 | Heterogeneous nuclear |
| | | | | | | ribonucleoprotein A3 |
| | ROA3_HUMAN | P51991 | | 91 | 1 | Heterogeneous nuclear |
| | | | | | | ribonucleoprotein A3 |
| | ROA3_HUMAN | P51991 | | 116 | 1 | Heterogeneous nuclear |
| | | | | | | ribonucleoprotein A3 |
| | ROCK1_HUMAN | Q13464 | | 1114 | 1 | Rho-associated protein |
| | | | | | | kinase 1 |
| | RPAP3_HUMAN | Q9H6T3 | | 125 | 1 | RNA polymerase II- |
| | | | | | | associated protein 3 |
| | RPAP3_HUMAN | Q9H6T3 | | 452 | 1 | RNA polymerase II- |
| | | | | | | associated protein 3 |
| | RPAP3_HUMAN | Q9H6T3 | | 452 | 1 | RNA polymerase II- |
| | | | | | | associated protein 3 |
| | RPB9_HUMAN | P36954 | | 5 | 1 | DNA-directed RNA |
| | | | | | | polymerase II subunit RPB9 |
| | RPC4_HUMAN | P05423 | | 132 | 1 | DNA-directed RNA |
| | | | | | | polymerase III subunit RPC4 |
| 263 | RPC5_HUMAN | Q9NVU0 | SFNGHPPQGCAST | 544 | 1 | DNA-directed RNA |
| | | | PVAR | | | polymerase III subunit RPC5 |
| | RPGF6_HUMAN | Q8TEU7 | | 1283 | 1 | Rap guanine nucleotide |
| | | | | | | exchange factor 6 |
| 264 | RPGF6_HUMAN | Q8TEU7 | SMSAALQDER | 1283 | 1 | Rap guanine nucleotide |
| | | | | | | exchange factor 6 |
| | RREB1_HUMAN | Q92766 | | 1174 | 1 | RAS-responsive element- |
| | | | | | | binding protein 1 |
| 265 | RRMJ3_HUMAN | Q8IY81 | STAGTTKQPSKEE | 347 | 1 | Putative rRNA |
| | | | EEEEEEEQLNQTL | | | methyltransferase 3 |
| | | | AEMK | | | |
| | RRP12_HUMAN | Q5JTH9 | | 1162 | 1 | RRP12-like protein |
| | RRP12_HUMAN | Q5JTH9 | | 1162 | 1 | RRP12-like protein |
| | RRP12_HUMAN | Q5JTH9 | | 1162 | 1 | RRP12-like protein |
| 266 | RRP12_HUMAN | Q5JTH9 | GNKMEEEEGAKG | 1162 | 1 | RRP12-like protein |
| | | | EDEEMADPMEDV | | | |
| | | | IIR | | | |
| | RRP12_HUMAN | Q5JTH9 | | 557 | 1 | RRP12-like protein |
| | RRP1B_HUMAN | Q14684 | | 276 | 1 | Ribosomal RNA processing |
| | | | | | | protein 1 homolog B |
| | RS20_HUMAN | P60866 | | 6 | 1 | 40S ribosomal protein S20 |
| | RS23_HUMAN | P62266 | | 89 | 1 | 40S ribosomal protein S23 |
| | RS23_HUMAN | P62266 | | 89 | 1 | 40S ribosomal protein S23 |
| | RS28_HUMAN | P62857 | | 55 | 1 | 40S ribosomal protein S28 |
| | RS3_HUMAN | P23396 | | 33 | 1 | 40S ribosomal protein S3 |
| 267 | RSRC1_HUMAN | Q96IZ7 | SFVQQTFR | 239 | 1 | Arginine/serine-rich coiled- |
| | | | | | | coil protein 1 |
| | RTF1_HUMAN | Q92541 | | 141 | 1 | RNA polymerase-associated |
| | | | | | | protein RTF1 homolog |
| 268 | RTF1_HUMAN | Q92541 | GYGEDLMGDEEDR | 141 | 1 | RNA polymerase-associated |
| | | | | | | protein RTF1 homolog |
| 269 | RTF1_HUMAN | Q92541 | GYGEDLMGDEED | 141 | 1 | RNA polymerase-associated |
| | | | RAR | | | protein RTF1 homolog |
| | RTN4_HUMAN | Q9NQC3 | | 85 | 1 | Reticulon-4 |
| | RTN4_HUMAN | Q9NQC3 | | 906 | 1 | Reticulon-4 |
| 270 | RU1C_HUMAN | P09234 | TYLTHDSPSVRK | 11 | 1 | U1 small nuclear |
| | | | | | | ribonucleoprotein C |
| 271 | RU1C_HUMAN | P09234 | TYLTHDSPSVR | 11 | 1 | U1 small nuclear |
| | | | | | | ribonucleoprotein C |
| 272 | RU2A_HUMAN | P09661 | AIDFSDNEIR | 46 | 1 | U2 small nuclear |
| | | | | | | ribonucleoprotein A′ |
| 273 | RUSD2_HUMAN | Q8IZ73 | STAPSSELGKDDL | 442 | 1 | RNA pseudouridylate |
| | | | EELAAAAQK | | | synthase domain-containing |
| | | | | | | protein 2 |
| | RUXF_HUMAN | P62306 | | 53 | 1 | Small nuclear |
| | | | | | | ribonucleoprotein F |
| | S11IP_HUMAN | Q8N1F8 | | 373 | 1 | Serine/threonine kinase 11- |
| | | | | | | interacting protein |
| | S12A2_HUMAN | P55011 | | 67 | 1 | Solute carrier family 12 |
| | | | | | | member 2 |
| | S2546_HUMAN | Q96AG3 | | 11 | 1 | Solute carrier family 25 |
| | | | | | | member 46 |
| 274 | S30BP_HUMAN | Q9UHR5 | AYGEDDFSR | 45 | 1 | SAP30-binding protein |
| | SAFB1_HUMAN | Q15424 | | 147 | 1 | Scaffold attachment factor |
| | | | | | | B1 |
| | SAFB1_HUMAN | Q15424 | | 797 | 2 | Scaffold attachment factor |
| | | | | | | B1 |
| | SAFB2_HUMAN | Q14151 | | 821 | | Scaffold attachment factor |
| | | | | | | B2 |
| | SAFB1_HUMAN | Q15424 | | 263 | 2 | Scaffold attachment factor |
| | | | | | | B1 |
| | SAFB2_HUMAN | Q14151 | | 262 | | Scaffold attachment factor |
| | | | | | | B2 |
| | SAFB1_HUMAN | Q15424 | | 263 | 2 | Scaffold attachment factor |
| | | | | | | B1 |
| | SAFB2_HUMAN | Q14151 | | 262 | | Scaffold attachment factor |
| | | | | | | B2 |
| | SAFB1_HUMAN | Q15424 | | 263 | 2 | Scaffold attachment factor |
| | | | | | | B1 |
| | SAFB2_HUMAN | Q14151 | | 262 | | Scaffold attachment factor |
| | | | | | | B2 |
| | SAFB1_HUMAN | Q15424 | | 361 | 2 | Scaffold attachment factor |
| | | | | | | B1 |
| | SAFB2_HUMAN | Q14151 | | 360 | | Scaffold attachment factor |
| | | | | | | B2 |
| | SAFB1_HUMAN | Q15424 | | 797 | 2 | Scaffold attachment factor |
| | | | | | | B1 |
| | SAFB2_HUMAN | Q14151 | | 821 | | Scaffold attachment factor |
| | | | | | | B2 |
| | SAFB2_HUMAN | Q14151 | | 184 | 1 | Scaffold attachment factor |
| | | | | | | B2 |
| | SAFB2_HUMAN | Q14151 | | 154 | 1 | Scaffold attachment factor |
| | | | | | | B2 |
| | SAFB2_HUMAN | Q14151 | | 154 | 1 | Scaffold attachment factor |
| | | | | | | B2 |
| | SAHH2_HUMAN | O43865 | | 6 | 1 | Putative |
| | | | | | | adenosylhomocysteinase 2 |
| 275 | SAHH2_HUMAN | O43865 | SYSSAASYTDSSD | 74 | 1 | Putative |
| | | | DEVSPR | | | adenosylhomocysteinase 2 |
| 276 | SAHH2_HUMAN | O43865 | SYSSAASYTDSSD | 74 | 1 | Putative |
| | | | DEVSPREK | | | adenosylhomocysteinase 2 |
| | SAHH2_HUMAN | O43865 | | 6 | 1 | Putative |
| | | | | | | adenosylhomocysteinase 2 |
| | SAHH2_HUMAN | O43865 | | 6 | 1 | Putative |
| | | | | | | adenosylhomocysteinase 2 |
| | SAHH2_HUMAN | O43865 | | 6 | 1 | Putative |
| | | | | | | adenosylhomocysteinase 2 |
| | SAHH2_HUMAN | O43865 | | 84 | 1 | Putative |
| | | | | | | adenosylhomocysteinase 2 |
| | SAHH3_HUMAN | Q96HN2 | | 110 | 1 | Putative |
| | | | | | | adenosylhomocysteinase 3 |
| | SAM4B_HUMAN | Q5PRF9 | | 413 | 1 | Sterile alpha motif domain- |
| | | | | | | containing protein 4B |
| | SAPS1_HUMAN | Q9UPN7 | | 359 | 1 | Serine/threonine-protein |
| | | | | | | phosphatase 6 regulatory |
| | | | | | | subunit 1 |
| 277 | SAP_HUMAN | P07602 | VYCEVCEFLVK | 313 | 1 | Proactivator polypeptide |
| 278 | SAP_HUMAN | P07602 | VYCEVCEFLVKE | 313 | 1 | Proactivator polypeptide |
| | | | VTK | | | |
| | SAP_HUMAN | P07602 | | 406 | 1 | Proactivator polypeptide |
| 279 | SASH3_HUMAN | O75995 | YSLDSPGPEK | 116 | 1 | SAM and SH3 domain- |
| | | | | | | containing protein 3 |
| | SASH3_HUMAN | O75995 | | 56 | 1 | SAM and SH3 domain- |
| | | | | | | containing protein 3 |
| | SATB1_HUMAN | Q01826 | | 255 | 1 | DNA-binding protein |
| | | | | | | SATB1 |
| | SATT_HUMAN | P43007 | | 13 | 1 | Neutral amino acid |
| | | | | | | transporter A |
| | SC16A_HUMAN | O15027 | | 838 | 1 | Protein transport protein |
| | | | | | | Sec16A |
| | SC16A_HUMAN | O15027 | | 342 | 1 | Protein transport protein |
| | | | | | | Sec16A |
| | SC24B_HUMAN | O95487 | | 296 | 1 | Protein transport protein |
| | | | | | | Sec24B |
| | SCAM3_HUMAN | O14828 | | 40 | 1 | Secretory carrier-associated |
| | | | | | | membrane protein 3 |
| | SCMH1_HUMAN | Q96GD3 | | 512 | 1 | Polycomb protein SCMH1 |
| | SCO1_HUMAN | O75880 | | 189 | 1 | Protein SCO1 homolog, |
| | | | | | | mitochondrial |
| | SCO1_HUMAN | O75880 | | 189 | 1 | Protein SCO1 homolog, |
| | | | | | | mitochondrial |
| | SCOC_HUMAN | Q9UIL1 | | 88 | 1 | Short coiled-coil protein |
| | SCOC_HUMAN | Q9UIL1 | | 88 | 1 | Short coiled-coil protein |
| | SDCG1_HUMAN | O60524 | | 780 | 1 | Serologically defined colon |
| | | | | | | cancer antigen 1 |
| | SEC13_HUMAN | P55735 | | 15 | 1 | Protein SEC13 homolog |
| | SEC20_HUMAN | Q12981 | | 33 | 1 | Vesicle transport protein |
| | | | | | | SEC20 |
| | SENP6_HUMAN | Q9GZR1 | | 50 | 1 | Sentrin-specific protease 6 |
| | SEPT9_HUMAN | Q9UHD8 | | 283 | 1 | Septin-9 |
| | SETD2_HUMAN | Q9BYW2 | | 648 | 1 | Histone-lysine N- |
| | | | | | | methyltransferase SETD2 |
| | SETD2_HUMAN | Q9BYW2 | | 1170 | 1 | Histone-lysine N- |
| | | | | | | methyltransferase SETD2 |
| | SETD2_HUMAN | Q9BYW2 | | 1170 | 1 | Histone-lysine N- |
| | | | | | | methyltransferase SETD2 |
| 280 | SETX_HUMAN | Q7Z333 | SVSRPQLESLSGTK | 1535 | 1 | Probable helicase senataxin |
| | SF01_HUMAN | Q15637 | | 449 | 1 | Splicing factor 1 |
| | SF3A1_HUMAN | Q15459 | | 504 | 1 | Splicing factor 3 subunit 1 |
| | SF3A1_HUMAN | Q15459 | | 504 | 1 | Splicing factor 3 subunit 1 |
| | SF3A1_HUMAN | Q15459 | | 33 | 1 | Splicing factor 3 subunit 1 |
| 281 | SF3B1_HUMAN | O75533 | STGYYDQEIYGGS | 35 | 1 | Splicing factor 3B subunit 1 |
| | | | DSR | | | |
| | SF3B2_HUMAN | Q13435 | | 292 | 1 | Splicing factor 3B subunit 2 |
| 282 | SF3B2_HUMAN | Q13435 | GSETPQLFTVLPEK | 754 | 1 | Splicing factor 3B subunit 2 |
| 283 | SF3B2_HUMAN | Q13435 | GSETPQLFTVLPE | 754 | 1 | Splicing factor 3B subunit 2 |
| | | | KR | | | |
| | SF3B4_HUMAN | Q15427 | | 13 | 1 | Splicing factor 3B subunit 4 |
| | SFPQ_HUMAN | P23246 | | 526 | 1 | Splicing factor, proline- and |
| | | | | | | glutamine-rich |
| | SFR14_HUMAN | Q8IX01 | | 733 | 1 | Putative splicing factor, |
| | | | | | | arginine/serine-rich 14 |
| | SFR14_HUMAN | Q8IX01 | | 902 | 1 | Putative splicing factor, |
| | | | | | | arginine/serine-rich 14 |
| 284 | SFR14_HUMAN | Q8IX01 | GLPGEAAEDDLA | 923 | 1 | Putative splicing factor, |
| | | | GAPALSQASSGTC | | | arginine/serine-rich 14 |
| | | | FPR | | | |
| 285 | SFRIP_HUMAN | Q99590 | SFCSDQNESEVEP | 408 | 1 | SFRS2-interacting protein |
| | | | SVNADLK | | | |
| | SFRS2_HUMAN | Q01130 | | 71 | 1 | Splicing factor, |
| | | | | | | arginine/serine-rich 2 |
| | SFRS2_HUMAN | Q01130 | | 71 | 1 | Splicing factor, |
| | | | | | | arginine/serine-rich 2 |
| | SFRS2_HUMAN | Q01130 | | 74 | 1 | Splicing factor, |
| | | | | | | arginine/serine-rich 2 |
| | SFRS2_HUMAN | Q01130 | | 74 | 1 | Splicing factor, |
| | | | | | | arginine/serine-rich 2 |
| | SFRS3_HUMAN | P84103 | | 5 | 1 | Splicing factor, |
| | | | | | | arginine/serine-rich 3 |
| | SFRS5_HUMAN | Q13243 | | 53 | 1 | Splicing factor, |
| | | | | | | arginine/serine-rich 5 |
| | SFRS6_HUMAN | Q13247 | | 168 | 1 | Splicing factor, |
| | | | | | | arginine/serine-rich 6 |
| | SGOL1_HUMAN | Q5FBB7 | | 207 | 1 | Shugoshin-like 1 |
| | SH2D3_HUMAN | Q8N5H7 | | 376 | 1 | SH2 domain-containing |
| | | | | | | protein 3C |
| | SHOT1_HUMAN | A0MZ66 | | 130 | 1 | Shootin-1 |
| 286 | SIPA1_HUMAN | Q96FS4 | GGSPPGPGDLAEER | 815 | 1 | Signal-induced proliferation- |
| | | | | | | associated protein 1 |
| | SIX4_HUMAN | Q9UIU6 | | 297 | 1 | Homeobox protein SIX4 |
| 287 | SKI_HUMAN | P12755 | AAAPADAPSGLE | 528 | 1 | Ski oncogene |
| | | | AELEHLR | | | |
| | SKT_HUMAN | Q5T5P2 | | 610 | 1 | Sickle tail protein homolog |
| | SLD5_HUMAN | Q9BRT9 | | 7 | 1 | DNA replication complex |
| | | | | | | GINS protein SLD5 |
| | SLK_HUMAN | Q9H2G2 | | 404 | 1 | STE20-like serine/threonine- |
| | | | | | | protein kinase |
| | SLMAP_HUMAN | Q14BN4 | | 465 | 1 | Sarcolemmal membrane- |
| | | | | | | associated protein |
| | SLU7_HUMAN | O95391 | | 8 | 1 | Pre-mRNA-splicing factor |
| | | | | | | SLU7 |
| | SLU7_HUMAN | O95391 | | 8 | 1 | Pre-mRNA-splicing factor |
| | | | | | | SLU7 |
| | SLU7_HUMAN | O95391 | | 8 | 1 | Pre-mRNA-splicing factor |
| | | | | | | SLU7 |
| | SLU7_HUMAN | O95391 | | 8 | 1 | Pre-mRNA-splicing factor |
| | | | | | | SLU7 |
| | SMC2_HUMAN | O95347 | | 1117 | 1 | Structural maintenance of |
| | | | | | | chromosomes protein 2 |
| | SMCA4_HUMAN | P51532 | | 1382 | 1 | Probable global transcription |
| | | | | | | activator SNF2L4 |
| | SMCE1_HUMAN | Q969G3 | | 265 | 1 | SWI/SNF-related matrix- |
| | | | | | | associated actin-dependent |
| | | | | | | regulator of chromatin |
| | | | | | | subfamily E member 1 |
| | SMHD1_HUMAN | A6NHR9 | | 6 | 1 | Structural maintenance of |
| | | | | | | chromosomes flexible hinge |
| | | | | | | domain-containing protein 1 |
| | SMRC2_HUMAN | Q8TAQ2 | | 815 | 1 | SWI/SNF complex subunit |
| | | | | | | SMARCC2 |
| | SMRD2_HUMAN | Q92925 | | 136 | 1 | SWI/SNF-related matrix- |
| | | | | | | associated actin-dependent |
| | | | | | | regulator of chromatin |
| | | | | | | subfamily D member 2 |
| | SNPC4_HUMAN | Q5SXM2 | | 1169 | 1 | snRNA-activating protein |
| | | | | | | complex subunit 4 |
| | SNX12_HUMAN | Q9UMY4 | | 22 | 1 | Sorting nexin-12 |
| 288 | SNX29_HUMAN | Q8TEQ0 | GEVTVAEQKPGEI | 183 | 1 | Sorting nexin-29 |
| | | | AEELASSYER | | | |
| | SNX2_HUMAN | O60749 | | 85 | 1 | Sorting nexin-2 |
| | SNX3_HUMAN | O60493 | | 33 | 1 | Sorting nexin-3 |
| | SNX6_HUMAN | Q9UNH7 | | 11 | 1 | Sorting nexin-6 |
| | SNX6_HUMAN | Q9UNH7 | | 11 | 1 | Sorting nexin-6 |
| | SOBP_HUMAN | A7XYQ1 | | 299 | 1 | Sine oculis-binding protein |
| | | | | | | homolog |
| 289 | SODC_HUMAN | P00441 | GVADVSIEDSVIS | 94 | 1 | Superoxide dismutase [Cu—Zn] |
| | | | LSGDHCIIGR | | | |
| 290 | SODC_HUMAN | P00441 | SVISLSGDHCIIGR | 103 | 1 | Superoxide dismutase [Cu—Zn] |
| | SON_HUMAN | P18583 | | 1641 | 1 | SON protein |
| 291 | SON_HUMAN | P18583 | SFLKFDSEPSAVA | 154 | 1 | SON protein |
| | | | LELPTR | | | |
| | SON_HUMAN | P18583 | | 1719 | 1 | SON protein |
| | SON_HUMAN | P18583 | | 1641 | 1 | SON protein |
| | SON_HUMAN | P18583 | | 353 | 1 | SON protein |
| | SP110_HUMAN | Q9HB58 | | 354 | 1 | Sp110 nuclear body protein |
| | SP110_HUMAN | Q9HB58 | | 354 | 1 | Sp110 nuclear body protein |
| | SP1_HUMAN | P08047 | | 200 | 1 | Transcription factor Sp1 |
| | SP3_HUMAN | Q02447 | | 276 | 1 | Transcription factor Sp3 |
| 292 | SP3_HUMAN | Q02447 | SAGIQLHPGENAD | 531 | 1 | Transcription factor Sp3 |
| | | | SPADIR | | | |
| | SPAS2_HUMAN | Q86XZ4 | | 146 | 1 | Spermatogenesis-associated |
| | | | | | | serine-rich protein 2 |
| | SPAST_HUMAN | Q9UBP0 | | 471 | 1 | Spastin |
| | SPD2B_HUMAN | A1X283 | | 683 | 1 | SH3 and PX domain- |
| | | | | | | containing protein 2B |
| | SPEC1_HUMAN | Q5M775 | | 214 | 1 | Sperm antigen with calponin |
| | | | | | | homology and coiled-coil |
| | | | | | | domains 1 |
| | SPEE_HUMAN | P19623 | | 7 | 1 | Spermidine synthase |
| | SPF27_HUMAN | O75934 | | 15 | 1 | Pre-mRNA-splicing factor |
| | | | | | | SPF27 |
| 293 | SPF30_HUMAN | O75940 | SFASTQPTHSWK | 63 | 1 | Survival of motor neuron- |
| | | | | | | related-splicing factor 30 |
| | SPG20_HUMAN | Q8N0X7 | | 497 | 1 | Spartin |
| | SPG20_HUMAN | Q8N0X7 | | 497 | 1 | Spartin |
| | SPG20_HUMAN | Q8N0X7 | | 497 | 1 | Spartin |
| | SPS2L_HUMAN | Q9NUQ6 | | 120 | 1 | SPATS2-like protein |
| 294 | SPT6H_HUMAN | Q7KZ85 | SYIEVLDGSR | 1048 | 1 | Transcription elongation |
| | | | | | | factor SPT6 |
| | SPTA2_HUMAN | Q13813 | | 1479 | 1 | Spectrin alpha chain, brain |
| | SPTA2_HUMAN | Q13813 | | 501 | 1 | Spectrin alpha chain, brain |
| | SPTN2_HUMAN | O15020 | | 1753 | 1 | Spectrin beta chain, brain 2 |
| 295 | SR140_HUMAN | O15042 | GAPLEDVDGIPID | 705 | 1 | U2-associated protein SR140 |
| | | | ATPIDDLDGVPIK | | | |
| | SR140_HUMAN | O15042 | | 713 | 1 | U2-associated protein SR140 |
| 296 | SR140_HUMAN | O15042 | GVPIKSLDDDLDG | 726 | 1 | U2-associated protein SR140 |
| | | | VPLDATEDSK | | | |
| 297 | SR140_HUMAN | O15042 | GVPIKSLDDDLDG | 726 | 1 | U2-associated protein SR140 |
| | | | VPLDATEDSKK | | | |
| 298 | SR140_HUMAN | O15042 | GVPLDATEDSK | 738 | 1 | U2-associated protein SR140 |
| 299 | SR140_HUMAN | O15042 | GVPLDATEDSKK | 738 | 1 | U2-associated protein SR140 |
| 300 | SR140_HUMAN | O15042 | GVPLDATEDSKK | 738 | 1 | U2-associated protein SR140 |
| | | | NEPIFK | | | |
| 301 | SRCAP_HUMAN | Q6ZRS2 | GFPAGEGEEAGRP | 2276 | 1 | Helicase SRCAP |
| | | | GAEDEEMSR | | | |
| 302 | SRCAP_HUMAN | Q6ZRS2 | GFPAGEGEEAGRP | 2276 | 1 | Helicase SRCAP |
| | | | GAEDEEMSR | | | |
| | SRC_HUMAN | P12931 | | 46 | 1 | Proto-oncogene tyrosine- |
| | | | | | | protein kinase Src |
| 303 | SRFB1_HUMAN | Q8NEF9 | SVVSLESQK | 212 | 1 | Serum response factor- |
| | | | | | | binding protein 1 |
| 304 | SRFB1_HUMAN | Q8NEF9 | SVVSLESQKTPAD | 212 | 1 | Serum response factor- |
| | | | PKLK | | | binding protein 1 |
| 305 | SRP68_HUMAN | Q9UHB9 | AHQTETSSSQVK | 538 | 1 | Signal recognition particle 68 kDa |
| | | | DNKPLVER | | | protein |
| 306 | SRP68_HUMAN | Q9UHB9 | AHQTETSSSQVK | 538 | 1 | Signal recognition particle 68 kDa |
| | | | | | | protein |
| | SRPK1_HUMAN | Q96SB4 | | 413 | 1 | Serine/threonine-protein |
| | | | | | | kinase SRPK1 |
| | SRRM2_HUMAN | Q9UQ35 | | 148 | 1 | Serine/arginine repetitive |
| | | | | | | matrix protein 2 |
| 307 | SRRM2_HUMAN | Q9UQ35 | SNSLLGQSR | 1150 | 1 | Serine/arginine repetitive |
| | | | | | | matrix protein 2 |
| | SRRM2_HUMAN | Q9UQ35 | | 148 | 1 | Serine/arginine repetitive |
| | | | | | | matrix protein 2 |
| | SSA27_HUMAN | O60232 | | 82 | 1 | Sjoegren |
| | | | | | | syndrome/scleroderma |
| | | | | | | autoantigen 1 |
| | SSBP3_HUMAN | Q9BWW4 | | 287 | 1 | Single-stranded DNA- |
| | | | | | | binding protein 3 |
| | SSF1_HUMAN | Q9NQ55 | | 246 | 1 | Suppressor of SWI4 1 |
| | | | | | | homolog |
| | SSFA2_HUMAN | P28290 | | 628 | 1 | Sperm-specific antigen 2 |
| | SSH2_HUMAN | Q76I76 | | 964 | 1 | Protein phosphatase |
| | | | | | | Slingshot homolog 2 |
| | SSRP1_HUMAN | Q08945 | | 174 | 1 | FACT complex subunit |
| | | | | | | SSRP1 |
| | STAP1_HUMAN | Q9ULZ2 | | 171 | 1 | Signal-transducing adaptor |
| | | | | | | protein 1 |
| 308 | STAP1_HUMAN | Q9ULZ2 | VLNPMPACFYTV | 171 | 1 | Signal-transducing adaptor |
| | | | SR | | | protein 1 |
| | STK10_HUMAN | O94804 | | 333 | 1 | Serine/threonine-protein |
| | | | | | | kinase 10 |
| | STK24_HUMAN | Q9Y6E0 | | 326 | 1 | Serine/threonine-protein |
| | | | | | | kinase 24 |
| | STK24_HUMAN | Q9Y6E0 | | 326 | 1 | Serine/threonine-protein |
| | | | | | | kinase 24 |
| | STK39_HUMAN | Q9UEW8 | | 436 | 1 | STE20/SPS1-related proline- |
| | | | | | | alanine-rich protein kinase |
| 309 | STK4_HUMAN | Q13043 | GANTMIEHDDTL | 350 | 1 | Serine/threonine-protein |
| | | | PSQLGTMVINAED | | | kinase 4 |
| | | | EEEEGTMK | | | |
| 310 | STK4_HUMAN | Q13043 | GANTMIEHDDTL | 350 | 1 | Serine/threonine-protein |
| | | | PSQLGTMVINAED | | | kinase 4 |
| | | | EEEEGTMKR | | | |
| 311 | STK4_HUMAN | Q13043 | GANTMIEHDDTL | 350 | 1 | Serine/threonine-protein |
| | | | PSQLGTMVINAED | | | kinase 4 |
| | | | EEEEGTMKRR | | | |
| | STRN_HUMAN | O43815 | | 36 | 1 | Striatin |
| 312 | STRN_HUMAN | O43815 | SLTYDIANNK | 437 | 1 | Striatin |
| 313 | STRN_HUMAN | O43815 | SLTYDIANNKDALR | 437 | 1 | Striatin |
| 314 | STRN_HUMAN | O43815 | SLTYDIANNKDAL | 437 | 1 | Striatin |
| | | | RK | | | |
| | STX10_HUMAN | O60499 | | 197 | 1 | Syntaxin-10 |
| | STX10_HUMAN | O60499 | | 139 | 1 | Syntaxin-10 |
| 315 | STX12_HUMAN | Q86Y82 | SIEANVESSEVHV | 218 | 1 | Syntaxin-12 |
| | | | ER | | | |
| | STX17_HUMAN | P56962 | | 202 | 1 | Syntaxin-17 |
| | STX17_HUMAN | P56962 | | 202 | 1 | Syntaxin-17 |
| 316 | STX7_HUMAN | O15400 | SIEANVENAEVHV | 205 | 1 | Syntaxin-7 |
| | | | QQANQQLSR | | | |
| 317 | SUGT1_HUMAN | Q9Y2Z0 | ALIDEDPQAALEE | 21 | 1 | Suppressor of G2 allele of |
| | | | LTK | | | SKP1 homolog |
| | SYAP1_HUMAN | Q96A49 | | 282 | 1 | Synapse-associated protein 1 |
| | SYEP_HUMAN | P07814 | | 930 | 1 | Bifunctional aminoacyl- |
| | | | | | | tRNA synthetase |
| 318 | SYF2_HUMAN | O95926 | SAEEGSLAAAAEL | 13 | 1 | Pre-mRNA-splicing factor |
| | | | AAQK | | | SYF2 |
| 319 | SYF2_HUMAN | O95926 | SAEEGSLAAAAEL | 13 | 1 | Pre-mRNA-splicing factor |
| | | | AAQKR | | | SYF2 |
| | SYG_HUMAN | P41250 | | 57 | 1 | Glycyl-tRNA synthetase |
| | SYMPK_HUMAN | Q92797 | | 29 | 1 | Symplekin |
| | SYNC_HUMAN | O43776 | | 410 | 1 | Asparaginyl-tRNA |
| | | | | | | synthetase, cytoplasmic |
| | SYNE1_HUMAN | Q8NF91 | | 8280 | 1 | Nesprin-1 |
| | SYNE2_HUMAN | Q8WXH0 | | 4216 | 1 | Nesprin-2 |
| 320 | SYWC_HUMAN | P23381 | FVDPWTVQTSSAK | 84 | 1 | Tryptophanyl-tRNA |
| | | | | | | synthetase, cytoplasmic |
| | T106B_HUMAN | Q9NUM4 | | 20 | 1 | Transmembrane protein |
| | | | | | | 106B |
| | T106B_HUMAN | Q9NUM4 | | 20 | 1 | Transmembrane protein |
| | | | | | | 106B |
| | T106C_HUMAN | Q9BVX2 | | 24 | 1 | Transmembrane protein |
| | | | | | | 106C |
| | T2EA_HUMAN | P29083 | | 304 | 1 | General transcription factor |
| | | | | | | IIE subunit 1 |
| 321 | T2EA_HUMAN | P29083 | AFQEREEGHAGP | 304 | 1 | General transcription factor |
| | | | DDNEEVMR | | | IIE subunit 1 |
| | T2FA_HUMAN | P35269 | | 273 | 1 | General transcription factor |
| | | | | | | IIF subunit 1 |
| | T2FA_HUMAN | P35269 | | 273 | 1 | General transcription factor |
| | | | | | | IIF subunit 1 |
| | TACC1_HUMAN | O75410 | | 324 | 1 | Transforming acidic coiled- |
| | | | | | | coil-containing protein 1 |
| 322 | TACC1_HUMAN | O75410 | GHATDEEKLASTS | 501 | 1 | Transforming acidic coiled- |
| | | | CGQK | | | coil-containing protein 1 |
| 323 | TACC1_HUMAN | O75410 | GHATDEEK | 501 | 1 | Transforming acidic coiled- |
| | | | | | | coil-containing protein 1 |
| | TACC2_HUMAN | O95359 | | 372 | 1 | Transforming acidic coiled- |
| | | | | | | coil-containing protein 2 |
| | TACC3_HUMAN | Q9Y6A5 | | 287 | 1 | Transforming acidic coiled- |
| | | | | | | coil-containing protein 3 |
| | TACC3_HUMAN | Q9Y6A5 | | 22 | 1 | Transforming acidic coiled- |
| | | | | | | coil-containing protein 3 |
| | TAD1L_HUMAN | Q96BN2 | | 79 | 1 | Transcriptional adapter 1- |
| | | | | | | like protein |
| 324 | TAF11_HUMAN | Q15544 | GIPEETDGDADVD | 35 | 1 | Transcription initiation factor |
| | | | LK | | | TFIID subunit 11 |
| | TAF7_HUMAN | Q15545 | | 101 | 1 | Transcription initiation factor |
| | | | | | | TFIID subunit 7 |
| | TBA1A_HUMAN | Q71U36 | | 34 | 5 | Tubulin alpha-1A chain |
| | TBA1B_HUMAN | P68363 | | 34 | | Tubulin alpha-1B chain |
| | TBA1C_HUMAN | Q9BQE3 | | 34 | | Tubulin alpha-1C chain |
| | TBA3C_HUMAN | Q13748 | | 34 | | Tubulin alpha-3C/D chain |
| | TBA3E_HUMAN | Q6PEY2 | | 34 | | Tubulin alpha-3E chain |
| | TBA1A_HUMAN | Q71U36 | | 34 | 5 | Tubulin alpha-1A chain |
| | TBA1B_HUMAN | P68363 | | 34 | | Tubulin alpha-1B chain |
| | TBA1C_HUMAN | Q9BQE3 | | 34 | | Tubulin alpha-1C chain |
| | TBA3C_HUMAN | Q13748 | | 34 | | Tubulin alpha-3C/D chain |
| | TBA3E_HUMAN | Q6PEY2 | | 34 | | Tubulin alpha-3E chain |
| | TBA1A_HUMAN | Q71U36 | | 34 | 5 | Tubulin alpha-1A chain |
| | TBA1B_HUMAN | P68363 | | 34 | | Tubulin alpha-1B chain |
| | TBA1C_HUMAN | Q9BQE3 | | 34 | | Tubulin alpha-1C chain |
| | TBA3C_HUMAN | Q13748 | | 34 | | Tubulin alpha-3C/D chain |
| | TBA3E_HUMAN | Q6PEY2 | | 34 | | Tubulin alpha-3E chain |
| | TBA1A_HUMAN | Q71U36 | | 48 | 5 | Tubulin alpha-1A chain |
| | TBA1B_HUMAN | P68363 | | 48 | | Tubulin alpha-1B chain |
| | TBA1C_HUMAN | Q9BQE3 | | 48 | | Tubulin alpha-1C chain |
| | TBA3C_HUMAN | Q13748 | | 48 | | Tubulin alpha-3C/D chain |
| | TBA3E_HUMAN | Q6PEY2 | | 48 | | Tubulin alpha-3E chain |
| | TBA1A_HUMAN | Q71U36 | | 34 | 6 | Tubulin alpha-1A chain |
| | TBA1B_HUMAN | P68363 | | 34 | | Tubulin alpha-1B chain |
| | TBA1C_HUMAN | Q9BQE3 | | 34 | | Tubulin alpha-1C chain |
| | TBA3C_HUMAN | Q13748 | | 34 | | Tubulin alpha-3C/D chain |
| | TBA3E_HUMAN | Q6PEY2 | | 34 | | Tubulin alpha-3E chain |
| | TBA4A_HUMAN | P68366 | | 34 | | Tubulin alpha-4A chain |
| | TBA1A_HUMAN | Q71U36 | | 200 | 7 | Tubulin alpha-1A chain |
| | TBA1B_HUMAN | P68363 | | 200 | | Tubulin alpha-1B chain |
| | TBA1C_HUMAN | Q9BQE3 | | 200 | | Tubulin alpha-1C chain |
| | TBA3C_HUMAN | Q13748 | | 200 | | Tubulin alpha-3C/D chain |
| | TBA3E_HUMAN | Q6PEY2 | | 200 | | Tubulin alpha-3E chain |
| | TBA4A_HUMAN | P68366 | | 200 | | Tubulin alpha-4A chain |
| | TBA8_HUMAN | Q9NY65 | | 200 | | Tubulin alpha-8 chain |
| | TBA1A_HUMAN | Q71U36 | | 246 | 7 | Tubulin alpha-1A chain |
| | TBA1B_HUMAN | P68363 | | 246 | | Tubulin alpha-1B chain |
| | TBA1C_HUMAN | Q9BQE3 | | 246 | | Tubulin alpha-1C chain |
| | TBA3C_HUMAN | Q13748 | | 246 | | Tubulin alpha-3C/D chain |
| | TBA3E_HUMAN | Q6PEY2 | | 246 | | Tubulin alpha-3E chain |
| | TBA4A_HUMAN | P68366 | | 246 | | Tubulin alpha-4A chain |
| | TBA8_HUMAN | Q9NY65 | | 246 | | Tubulin alpha-8 chain |
| | TBB2A_HUMAN | Q13885 | | 115 | 5 | Tubulin beta-2A chain |
| | TBB2B_HUMAN | Q9BVA1 | | 115 | | Tubulin beta-2B chain |
| | TBB2C_HUMAN | P68371 | | 115 | | Tubulin beta-2C chain |
| | TBB3_HUMAN | Q13509 | | 115 | | Tubulin beta-3 chain |
| | TBB5_HUMAN | P07437 | | 115 | | Tubulin beta chain |
| | TBB2A_HUMAN | Q13885 | | 115 | 5 | Tubulin beta-2A chain |
| | TBB2B_HUMAN | Q9BVA1 | | 115 | | Tubulin beta-2B chain |
| | TBB2C_HUMAN | P68371 | | 115 | | Tubulin beta-2C chain |
| | TBB3_HUMAN | Q13509 | | 115 | | Tubulin beta-3 chain |
| | TBB5_HUMAN | P07437 | | 115 | | Tubulin beta chain |
| | TBB2C_HUMAN | P68371 | | 115 | 2 | Tubulin beta-2C chain |
| | TBB5_HUMAN | P07437 | | 115 | | Tubulin beta chain |
| | TBCC_HUMAN | Q15814 | | 154 | 1 | Tubulin-specific chaperone C |
| | TBCD4_HUMAN | O60343 | | 273 | 1 | TBC1 domain family |
| | | | | | | member 4 |
| | TBCD4_HUMAN | O60343 | | 276 | 1 | TBC1 domain family |
| | | | | | | member 4 |
| | TBL1R_HUMAN | Q9BZK7 | | 153 | 1 | F-box-like/WD repeat- |
| | | | | | | containing protein TBL1XR1 |
| 325 | TBL1R_HUMAN | Q9BZK7 | AVMPDVVQTR | 86 | 2 | F-box-like/WD repeat- |
| | | | | | | containing protein TBL1XR1 |
| 325 | TBL1X_HUMAN | O60907 | AVMPDVVQTR | 86 | | F-box-like/WD repeat- |
| | | | | | | containing protein TBL1X |
| | TBL1R_HUMAN | Q9BZK7 | | 153 | 2 | F-box-like/WD repeat- |
| | | | | | | containing protein TBL1XR1 |
| | TBL1Y_HUMAN | Q9BQ87 | | 163 | | F-box-like/WD repeat- |
| | | | | | | containing protein TBL1Y |
| | TBL1X_HUMAN | O60907 | | 165 | 1 | F-box-like/WD repeat- |
| | | | | | | containing protein TBL1X |
| | TCEA1_HUMAN | P23193 | | 125 | 1 | Transcription elongation |
| | | | | | | factor A protein 1 |
| | TCF20_HUMAN | Q9UGU0 | | 1220 | 1 | Transcription factor 20 |
| | TCF20_HUMAN | Q9UGU0 | | 1220 | 1 | Transcription factor 20 |
| | TCF20_HUMAN | Q9UGU0 | | 1220 | 1 | Transcription factor 20 |
| | TCOF_HUMAN | Q13428 | | 1243 | 1 | Treacle protein |
| 326 | TCOF_HUMAN | Q13428 | GKQEAKPQQAAG | 1243 | 1 | Treacle protein |
| | | | MLSPK | | | |
| | TCOF_HUMAN | Q13428 | | 1102 | 1 | Treacle protein |
| | TCPD_HUMAN | P50991 | | 457 | 1 | T-complex protein 1 subunit |
| | | | | | | delta |
| | TCPD_HUMAN | P50991 | | 269 | 2 | T-complex protein 1 subunit |
| | | | | | | delta |
| | TCPD_HUMAN | P50991 | | 269 | 2 | T-complex protein 1 subunit |
| | | | | | | delta |
| | TCPE_HUMAN | P48643 | | 66 | 1 | T-complex protein 1 subunit |
| | | | | | | epsilon |
| | TCPE_HUMAN | P48643 | | 154 | 1 | T-complex protein 1 subunit |
| | | | | | | epsilon |
| | TCPZ_HUMAN | P40227 | | 405 | 1 | T-complex protein 1 subunit |
| | | | | | | zeta |
| | TCTP_HUMAN | P13693 | | 26 | 1 | Translationally-controlled |
| | | | | | | tumor protein |
| | TDRD6_HUMAN | O60522 | | 1919 | 1 | Tudor domain-containing |
| | | | | | | protein 6 |
| | TEX2_HUMAN | Q8IWB9 | | 357 | 1 | Testis-expressed sequence 2 |
| | | | | | | protein |
| 327 | TEX2_HUMAN | Q8IWB9 | GLSVSQAPAILPV | 97 | 1 | Testis-expressed sequence 2 |
| | | | SK | | | protein |
| | TF2B_HUMAN | Q00403 | | 208 | 1 | Transcription initiation factor |
| | | | | | | IIB |
| | TF2B_HUMAN | Q00403 | | 208 | 1 | Transcription initiation factor |
| | | | | | | IIB |
| | TF2L1_HUMAN | Q9NZI6 | | 23 | 3 | Transcription factor CP2-like |
| | | | | | | protein 1 |
| | TFCP2_HUMAN | Q12800 | | 43 | | Alpha-globin transcription |
| | | | | | | factor CP2 |
| | UBIP1_HUMAN | Q9NZI7 | | 40 | | Upstream-binding protein 1 |
| 328 | TF3A_HUMAN | Q92664 | AFIAAGESSAPTPP | 19 | 1 | Transcription factor IIIA |
| | | | RPALPR | | | |
| | TF65_HUMAN | Q04206 | | 98 | 1 | Transcription factor p65 |
| | TGS1_HUMAN | Q96RS0 | | 344 | 1 | Trimethylguanosine synthase |
| | | | | | | homolog |
| | TGS1_HUMAN | Q96RS0 | | 344 | 1 | Trimethylguanosine synthase |
| | | | | | | homolog |
| | TGS1_HUMAN | Q96RS0 | | 338 | 1 | Trimethylguanosine synthase |
| | | | | | | homolog |
| | THOC4_HUMAN | Q86V81 | | 94 | 1 | THO complex subunit 4 |
| | THOC5_HUMAN | Q13769 | | 18 | 1 | THO complex subunit 5 |
| | | | | | | homolog |
| | THOP1_HUMAN | P52888 | | 14 | 1 | Thimet oligopeptidase |
| | TIF1A_HUMAN | O15164 | | 785 | 1 | Transcription intermediary |
| | | | | | | factor 1-alpha |
| 329 | TIF1B_HUMAN | Q13263 | ANQCCTSCEDNA | 149 | 1 | Transcription intermediary |
| | | | PATSYCVECSEPL | | | factor 1-beta |
| | | | CETCVEAHQR | | | |
| | TIF1B_HUMAN | Q13263 | | 106 | 1 | Transcription intermediary |
| | | | | | | factor 1-beta |
| 330 | TIF1B_HUMAN | Q13263 | STFSLDQPGGTLD | 727 | 1 | Transcription intermediary |
| | | | LTLIR | | | factor 1-beta |
| | TIF1B_HUMAN | Q13263 | | 686 | 1 | Transcription intermediary |
| | | | | | | factor 1-beta |
| | TIF1B_HUMAN | Q13263 | | 689 | 1 | Transcription intermediary |
| | | | | | | factor 1-beta |
| 331 | TIM_HUMAN | Q9UNS1 | SVVPFDAASEVPV | 580 | 1 | Protein timeless homolog |
| | | | EEQR | | | |
| | TINF2_HUMAN | Q9BSI4 | | 208 | 1 | TERF1-interacting nuclear |
| | | | | | | factor 2 |
| 332 | TINF2_HUMAN | Q9BSI4 | SVNLAEPMEQNP | 208 | 1 | TERF1-interacting nuclear |
| | | | PQQQR | | | factor 2 |
| | TLK2_HUMAN | Q86UE8 | | 133 | 1 | Serine/threonine-protein |
| | | | | | | kinase tousled-like 2 |
| | TM168_HUMAN | Q9H0V1 | | 427 | 1 | Transmembrane protein 168 |
| | TM1L2_HUMAN | Q6ZVM7 | | 158 | 1 | TOM1-like protein 2 |
| | TMUB1_HUMAN | Q9BVT8 | | 61 | 1 | Transmembrane and |
| | | | | | | ubiquitin-like domain- |
| | | | | | | containing protein 1 |
| 333 | TMUB1_HUMAN | Q9BVT8 | SMRGEAPGAETPS | 61 | 1 | Transmembrane and |
| | | | LR | | | ubiquitin-like domain- |
| | | | | | | containing protein 1 |
| | TNIP2_HUMAN | Q8NFZ5 | | 195 | 1 | TNFAIP3-interacting protein 2 |
| | TNR6A_HUMAN | Q8NDV7 | | 1543 | 1 | Trinucleotide repeat- |
| | | | | | | containing gene 6A protein |
| | TNR6A_HUMAN | Q8NDV7 | | 1543 | 1 | Trinucleotide repeat- |
| | | | | | | containing gene 6A protein |
| 334 | TOE1_HUMAN | Q96GM8 | SIKPEETEQEVAA | 374 | 1 | Target of EGR1 protein 1 |
| | | | DETR | | | |
| | TOE1_HUMAN | Q96GM8 | | 8 | 1 | Target of EGR1 protein 1 |
| 335 | TOIP1_HUMAN | Q5JTV8 | SILKSELGNQSPST | 305 | 1 | Torsin-1A-interacting protein 1 |
| | | | SSR | | | |
| | TOIP1_HUMAN | Q5JTV8 | | 227 | 1 | Torsin-1A-interacting protein 1 |
| | TOIP1_HUMAN | Q5JTV8 | | 227 | 1 | Torsin-1A-interacting protein 1 |
| | TOLIP_HUMAN | Q9H0E2 | | 37 | 1 | Toll-interacting protein |
| | TOM1_HUMAN | O60784 | | 394 | 1 | Target of Myb protein 1 |
| | TOM1_HUMAN | O60784 | | 185 | 1 | Target of Myb protein 1 |
| | TOM1_HUMAN | O60784 | | 180 | 1 | Target of Myb protein 1 |
| | TOM1_HUMAN | O60784 | | 158 | 1 | Target of Myb protein 1 |
| 336 | TOM1_HUMAN | O60784 | MLSPIHTPQR | 158 | 1 | Target of Myb protein 1 |
| | TOP2B_HUMAN | Q02880 | | 1471 | 1 | DNA topoisomerase 2-beta |
| | TP53B_HUMAN | Q12888 | | 212 | 1 | Tumor suppressor p53- |
| | | | | | | binding protein 1 |
| 337 | TP53B_HUMAN | Q12888 | GCSTPSREEGGCS | 318 | 1 | Tumor suppressor p53- |
| | | | LASTPATTLHLLQ | | | binding protein 1 |
| | | | LSGQR | | | |
| | TP53B_HUMAN | Q12888 | | 1479 | 1 | Tumor suppressor p53- |
| | | | | | | binding protein 1 |
| | TP53B_HUMAN | Q12888 | | 318 | 1 | Tumor suppressor p53- |
| | | | | | | binding protein 1 |
| 338 | TP53B_HUMAN | Q12888 | SSQPSLPLVR | 830 | 1 | Tumor suppressor p53- |
| | | | | | | binding protein 1 |
| | TPD54_HUMAN | O43399 | | 3 | 1 | Tumor protein D54 |
| 339 | TPRGL_HUMAN | Q5T0D9 | SAGTSPTAVLAA | 10 | 1 | Tumor protein p63-regulated |
| | | | GEEVGAGGGPGG | | | gene 1-like protein |
| | | | GRPGAGTPLR | | | |
| 340 | TPRGL_HUMAN | Q5T0D9 | SAGTSPTAVLAA | 10 | 1 | Tumor protein p63-regulated |
| | | | GEEVGAGGGPGG | | | gene 1-like protein |
| | | | GRPGAGTPLRQTL | | | |
| | | | WPLSIHDPTR | | | |
| | TPR_HUMAN | P12270 | | 1838 | 1 | Nucleoprotein TPR |
| | TPR_HUMAN | P12270 | | 2148 | 1 | Nucleoprotein TPR |
| 341 | TR150_HUMAN | Q9Y2W1 | SFDEDLARPSGLL | 575 | 1 | Thyroid hormone receptor- |
| | | | AQER | | | associated protein 3 |
| | TRBP2_HUMAN | Q15633 | | 235 | 1 | TAR RNA-binding protein 2 |
| 342 | TREF1_HUMAN | Q96PN7 | GSNVTVTPGPGE | 761 | 1 | Transcriptional-regulating |
| | | | QTVDVEPR | | | factor 1 |
| | TRI33_HUMAN | Q9UPN9 | | 830 | 1 | E3 ubiquitin-protein ligase |
| | | | | | | TRIM33 |
| | TRI33_HUMAN | Q9UPN9 | | 830 | 1 | E3 ubiquitin-protein ligase |
| | | | | | | TRIM33 |
| | TRI33_HUMAN | Q9UPN9 | | 830 | 1 | E3 ubiquitin-protein ligase |
| | | | | | | TRIM33 |
| | TRIP4_HUMAN | Q15650 | | 123 | 1 | Activating signal |
| | | | | | | cointegrator 1 |
| | TRIP4_HUMAN | Q15650 | | 289 | 1 | Activating signal |
| | | | | | | cointegrator 1 |
| | TRM1L_HUMAN | Q7Z2T5 | | 45 | 1 | TRM1-like protein |
| | TRS85_HUMAN | Q9Y2L5 | | 854 | 1 | Protein TRS85 homolog |
| | TSC1_HUMAN | Q92574 | | 639 | 1 | Hamartin |
| 343 | TSC1_HUMAN | Q92574 | GVPSTSPMEVLDR | 639 | 1 | Hamartin |
| | | | LIQQGADAHSK | | | |
| 344 | TSC1_HUMAN | Q92574 | GVPSTSPMEVLDR | 639 | 1 | Hamartin |
| | TSR1_HUMAN | Q2NL82 | | 333 | 1 | Pre-rRNA-processing protein |
| | | | | | | TSR1 homolog |
| | TSR1_HUMAN | Q2NL82 | | 333 | 1 | Pre-rRNA-processing protein |
| | | | | | | TSR1 homolog |
| 345 | TSR1_HUMAN | Q2NL82 | AVDDMEEGLK | 333 | 1 | Pre-rRNA-processing protein |
| | | | | | | TSR1 homolog |
| | TSR1_HUMAN | Q2NL82 | | 333 | 1 | Pre-rRNA-processing protein |
| | | | | | | TSR1 homolog |
| 346 | TSR1_HUMAN | Q2NL82 | AVDDMEEGLKVL | 333 | 1 | Pre-rRNA-processing protein |
| | | | MK | | | TSR1 homolog |
| | TTC1_HUMAN | Q99614 | | 66 | 1 | Tetratricopeptide repeat |
| | | | | | | protein 1 |
| | TTC4_HUMAN | O95801 | | 255 | 1 | Tetratricopeptide repeat |
| | | | | | | protein 4 |
| 347 | TTF2_HUMAN | Q9UNY4 | STGRPLVILPQR | 827 | 1 | Transcription termination |
| | | | | | | factor 2 |
| 348 | TTF2_HUMAN | Q9UNY4 | STGRPLVILPQRK | 827 | 1 | Transcription termination |
| | | | | | | factor 2 |
| | TYB10_HUMAN | P63313 | | 7 | 1 | Thymosin beta-10 |
| | TYSY_HUMAN | P04818 | | 120 | 1 | Thymidylate synthase |
| | TYY1_HUMAN | P25490 | | 120 | 1 | Transcriptional repressor |
| | | | | | | protein YY1 |
| | U119A_HUMAN | Q13432 | | 45 | 1 | Protein unc-119 homolog A |
| | U119B_HUMAN | A6NIH7 | | 52 | 1 | Protein unc-119 homolog B |
| | U2AF2_HUMAN | P26368 | | 129 | 1 | Splicing factor U2AF 65 kDa |
| | | | | | | subunit |
| 349 | U2AF2_HUMAN | P26368 | GLAVTPTPVPVV | 129 | 1 | Splicing factor U2AF 65 kDa |
| | | | GSQMTR | | | subunit |
| 350 | UAP1L_HUMAN | Q3KQV9 | GVPQVVEYSEISP | 300 | 1 | UDP-N-acetylhexosamine |
| | | | ETAQLR | | | pyrophosphorylase-like |
| | | | | | | protein 1 |
| | UAP56_HUMAN | Q13838 | | 26 | 1 | Spliceosome RNA helicase |
| | | | | | | BAT1 |
| 351 | UBA1_HUMAN | P22314 | ALECLPEDKEVLT | 428 | 1 | Ubiquitin-like modifier- |
| | | | EDK | | | activating enzyme 1 |
| | UBA3_HUMAN | Q8TBC4 | | 26 | 1 | NEDD8-activating enzyme |
| | | | | | | E1 catalytic subunit |
| | UBA3_HUMAN | Q8TBC4 | | 26 | 1 | NEDD8-activating enzyme |
| | | | | | | E1 catalytic subunit |
| | UBAP2_HUMAN | Q5T6F2 | | 855 | 1 | Ubiquitin-associated protein 2 |
| | UBAP2_HUMAN | Q5T6F2 | | 202 | 1 | Ubiquitin-associated protein 2 |
| | UBAP2_HUMAN | Q5T6F2 | | 263 | 1 | Ubiquitin-associated protein 2 |
| | UBE2O_HUMAN | Q9C0C9 | | 438 | 1 | Ubiquitin-conjugating |
| | | | | | | enzyme E2 O |
| 352 | UBE2O_HUMAN | Q9C0C9 | GSASPVEMQDEG | 438 | 1 | Ubiquitin-conjugating |
| | | | AEEPHEAGEQLPP | | | enzyme E2 O |
| | | | FLLK | | | |
| 353 | UBE2O_HUMAN | Q9C0C9 | GSASPVEMQDEG | 438 | 1 | Ubiquitin-conjugating |
| | | | AEEPHEAGEQLPP | | | enzyme E2 O |
| | | | FLLKEGR | | | |
| 354 | UBE2O_HUMAN | Q9C0C9 | GSASPVEMQDEG | 438 | 1 | Ubiquitin-conjugating |
| | | | AEEPHEAGEQLPP | | | enzyme E2 O |
| | | | FLLKEGRDDR | | | |
| | UBE2O_HUMAN | Q9C0C9 | | 1226 | 1 | Ubiquitin-conjugating |
| | | | | | | enzyme E2 O |
| | UBFD1_HUMAN | O14562 | | 233 | 1 | Ubiquitin domain-containing |
| | | | | | | protein UBFD1 |
| 355 | UBN1_HUMAN | Q9NPG3 | SFIDNSEAYDELV | 137 | 1 | Ubinuclein |
| | | | PASLTTK | | | |
| | UBP10_HUMAN | Q14694 | | 126 | 1 | Ubiquitin carboxyl-terminal |
| | | | | | | hydrolase 10 |
| | UBP10_HUMAN | Q14694 | | 139 | 1 | Ubiquitin carboxyl-terminal |
| | | | | | | hydrolase 10 |
| | UBP10_HUMAN | Q14694 | | 218 | 1 | Ubiquitin carboxyl-terminal |
| | | | | | | hydrolase 10 |
| | UBP14_HUMAN | P54578 | | 77 | 1 | Ubiquitin carboxyl-terminal |
| | | | | | | hydrolase 14 |
| | UBP14_HUMAN | P54578 | | 228 | 1 | Ubiquitin carboxyl-terminal |
| | | | | | | hydrolase 14 |
| 356 | UBP19_HUMAN | O94966 | GRPDEVVAEEAW | 620 | 1 | Ubiquitin carboxyl-terminal |
| | | | QR | | | hydrolase 19 |
| | UBP2L_HUMAN | Q14157 | | 412 | 1 | Ubiquitin-associated protein |
| | | | | | | 2-like |
| | UBP2L_HUMAN | Q14157 | | 299 | 1 | Ubiquitin-associated protein |
| | | | | | | 2-like |
| 357 | UBP2L_HUMAN | Q14157 | GSLASNPYSGDLTK | 851 | 1 | Ubiquitin-associated protein |
| | | | | | | 2-like |
| | UBP34_HUMAN | Q70CQ2 | | 3367 | 1 | Ubiquitin carboxyl-terminal |
| | | | | | | hydrolase 34 |
| | UBP34_HUMAN | Q70CQ2 | | 3367 | 1 | Ubiquitin carboxyl-terminal |
| | | | | | | hydrolase 34 |
| | UBP36_HUMAN | Q9P275 | | 577 | 1 | Ubiquitin carboxyl-terminal |
| | | | | | | hydrolase 36 |
| | UBP42_HUMAN | Q9H9J4 | | 765 | 1 | Ubiquitin carboxyl-terminal |
| | | | | | | hydrolase 42 |
| | UBP5_HUMAN | P45974 | | 768 | 1 | Ubiquitin carboxyl-terminal |
| | | | | | | hydrolase 5 |
| | UBP5_HUMAN | P45974 | | 768 | 1 | Ubiquitin carboxyl-terminal |
| | | | | | | hydrolase 5 |
| | UBP5_HUMAN | P45974 | | 783 | 1 | Ubiquitin carboxyl-terminal |
| | | | | | | hydrolase 5 |
| | UBP5_HUMAN | P45974 | | 135 | 1 | Ubiquitin carboxyl-terminal |
| | | | | | | hydrolase 5 |
| | UBP5_HUMAN | P45974 | | 783 | 1 | Ubiquitin carboxyl-terminal |
| | | | | | | hydrolase 5 |
| | UBP7_HUMAN | Q93009 | | 51 | 1 | Ubiquitin carboxyl-terminal |
| | | | | | | hydrolase 7 |
| 358 | UBP7_HUMAN | Q93009 | GHNTAEEDMEDD | 51 | 1 | Ubiquitin carboxyl-terminal |
| | | | TSWR | | | hydrolase 7 |
| | UBQL1_HUMAN | Q9UMX0 | | 16 | 1 | Ubiquilin-1 |
| 359 | UBR4_HUMAN | Q5T4S7 | SVAGEHSVSGR | 2904 | 1 | E3 ubiquitin-protein ligase |
| | | | | | | UBR4 |
| 360 | UBXN7_HUMAN | O94888 | GFRDFQTETIR | 110 | 1 | UBX domain-containing |
| | | | | | | protein 7 |
| 361 | UBXN7_HUMAN | O94888 | GFRDFQTETIRQE | 110 | 1 | UBX domain-containing |
| | | | QELR | | | protein 7 |
| | UBXN7_HUMAN | O94888 | | 401 | 1 | UBX domain-containing |
| | | | | | | protein 7 |
| | UGPA_HUMAN | Q16851 | | 16 | 1 | UTP--glucose-1-phosphate |
| | | | | | | uridylyltransferase |
| | UH1BL_HUMAN | A0JNW5 | | 1174 | 1 | UHRF1-binding protein 1- |
| | | | | | | like |
| 362 | UHRF1_HUMAN | Q96T88 | SRPADEDMWDET | 119 | 1 | E3 ubiquitin-protein ligase |
| | | | ELGLYK | | | UHRF1 |
| 363 | UHRF1_HUMAN | Q96T88 | SRPADEDMWDET | 119 | 1 | E3 ubiquitin-protein ligase |
| | | | ELGLYKVNEYVD | | | UHRF1 |
| | | | AR | | | |
| 364 | URP2_HUMAN | Q86UX7 | SLTTIPELK | 345 | 1 | Fermitin family homolog 3 |
| 365 | URP2_HUMAN | Q86UX7 | SLTTIPELKDHLR | 345 | 1 | Fermitin family homolog 3 |
| | USE1_HUMAN | Q9NZ43 | | 130 | 1 | Vesicle transport protein |
| | | | | | | USE1 |
| | USF2_HUMAN | Q15853 | | 121 | 1 | Upstream stimulatory factor 2 |
| | USO1_HUMAN | O60763 | | 758 | 1 | General vesicular transport |
| | | | | | | factor p115 |
| | UTRO_HUMAN | P46939 | | 262 | 1 | Utrophin |
| | VAMP2_HUMAN | P63027 | | 69 | 2 | Vesicle-associated |
| | | | | | | membrane protein 2 |
| | VAMP3_HUMAN | Q15836 | | 52 | | Vesicle-associated |
| | | | | | | membrane protein 3 |
| | VATD_HUMAN | Q9Y5K8 | | 118 | 1 | V-type proton ATPase |
| | | | | | | subunit D |
| 366 | VIME_HUMAN | P08670 | AINTEFK | 91 | 1 | Vimentin |
| 367 | VIME_HUMAN | P08670 | AINTEFKNTR | 91 | 1 | Vimentin |
| 368 | VIME_HUMAN | P08670 | ALKGTNESLER | 332 | 1 | Vimentin |
| 369 | VIME_HUMAN | P08670 | FSLADAINTEFK | 86 | 1 | Vimentin |
| 370 | VIME_HUMAN | P08670 | FSLADAINTEFKN | 86 | 1 | Vimentin |
| | | | TR | | | |
| | VIME_HUMAN | P08670 | | 83 | 1 | Vimentin |
| | VIME_HUMAN | P08670 | | 430 | 1 | Vimentin |
| 371 | VIME_HUMAN | P08670 | VDVSKPDLTAALR | 258 | 1 | Vimentin |
| 372 | VIME_HUMAN | P08670 | VDVSKPDLTAAL | 258 | 1 | Vimentin |
| | | | RDVR | | | |
| 373 | VIME_HUMAN | P08670 | VSKPDLTAALR | 260 | 1 | Vimentin |
| 374 | VIME_HUMAN | P08670 | VSKPDLTAALRD | 260 | 1 | Vimentin |
| | | | VR | | | |
| 375 | VP13D_HUMAN | Q5THJ4 | SVGTYLPGASR | 2611 | 1 | Vacuolar protein sorting- |
| | | | | | | associated protein 13D |
| 376 | VPS4A_HUMAN | Q9UN37 | SLCGSRNENESEA | 231 | 1 | Vacuolar protein sorting- |
| | | | AR | | | associating protein 4A |
| 377 | VPS4A_HUMAN | Q9UN37 | SLCGSRNENESEA | 231 | 1 | Vacuolar protein sorting- |
| | | | ARR | | | associating protein 4A |
| | VRK1_HUMAN | Q99986 | | 232 | 1 | Serine/threonine-protein |
| | | | | | | kinase VRK1 |
| | WAPL_HUMAN | Q7Z5K2 | | 155 | 1 | Wings apart-like protein |
| | | | | | | homolog |
| | WASF1_HUMAN | Q92558 | | 248 | 1 | Wiskott-Aldrich syndrome |
| | | | | | | protein family member 1 |
| | WASF2_HUMAN | Q9Y6W5 | | 243 | 1 | Wiskott-Aldrich syndrome |
| | | | | | | protein family member 2 |
| | WASF2_HUMAN | Q9Y6W5 | | 412 | 1 | Wiskott-Aldrich syndrome |
| | | | | | | protein family member 2 |
| | WASH1_HUMAN | A8K0Z3 | | 299 | 1 | WAS protein family |
| | | | | | | homolog 1 |
| | WDR33_HUMAN | Q9C0J8 | | 1184 | 1 | WD repeat-containing |
| | | | | | | protein 33 |
| | WDR44_HUMAN | Q5JSH3 | | 84 | 1 | WD repeat-containing |
| | | | | | | protein 44 |
| | WDR55_HUMAN | Q9H6Y2 | | 21 | 1 | WD repeat-containing |
| | | | | | | protein 55 |
| | WDR62_HUMAN | O43379 | | 1302 | 1 | WD repeat-containing |
| | | | | | | protein 62 |
| 378 | WDR92_HUMAN | Q96MX6 | GIGGLGIGEGAPEI | 119 | 1 | WD repeat-containing |
| | | | VTGSR | | | protein 92 |
| | WFS1_HUMAN | O76024 | | 212 | 1 | Wolframin |
| | WFS1_HUMAN | O76024 | | 76 | 1 | Wolframin |
| | WIPF1_HUMAN | O43516 | | 182 | 1 | WAS/WASL-interacting |
| | | | | | | protein family member 1 |
| | WNK1_HUMAN | Q9H4A3 | | 1070 | 1 | Serine/threonine-protein |
| | | | | | | kinase WNK1 |
| | WNK1_HUMAN | Q9H4A3 | | 1070 | 1 | Serine/threonine-protein |
| | | | | | | kinase WNK1 |
| | WNK1_HUMAN | Q9H4A3 | | 1070 | 1 | Serine/threonine-protein |
| | | | | | | kinase WNK1 |
| | WNK1_HUMAN | Q9H4A3 | | 2026 | 1 | Serine/threonine-protein |
| | | | | | | kinase WNK1 |
| | WNK1_HUMAN | Q9H4A3 | | 653 | 1 | Serine/threonine-protein |
| | | | | | | kinase WNK1 |
| | WNK1_HUMAN | Q9H4A3 | | 1070 | 1 | Serine/threonine-protein |
| | | | | | | kinase WNK1 |
| | WRIP1_HUMAN | Q96S55 | | 193 | 1 | ATPase WRNIP1 |
| | WWC2_HUMAN | Q6AWC2 | | 856 | 1 | Protein WWC2 |
| | XPA_HUMAN | P23025 | | 6 | 1 | DNA repair protein |
| | | | | | | complementing XP-A cells |
| | YAP1_HUMAN | P46937 | | 112 | 1 | 65 kDa Yes-associated |
| | | | | | | protein |
| | YBOX1_HUMAN | P67809 | | 25 | 1 | Nuclease-sensitive element- |
| | | | | | | binding protein 1 |
| | YBOX1_HUMAN | P67809 | | 25 | 1 | Nuclease-sensitive element- |
| | | | | | | binding protein 1 |
| | YBOX1_HUMAN | P67809 | | 113 | 1 | Nuclease-sensitive element- |
| | | | | | | binding protein 1 |
| | YIPF3_HUMAN | Q9GZM5 | | 69 | 1 | Protein YIPF3 |
| | YJ005_HUMAN | Q6ZSR9 | | 118 | 1 | Uncharacterized protein |
| | | | | | | FLJ45252 |
| | YJ005_HUMAN | Q6ZSR9 | | 124 | 1 | Uncharacterized protein |
| | | | | | | FLJ45252 |
| | YM017_HUMAN | A8MX80 | | 224 | 1 | Putative UPF0607 protein |
| | | | | | | ENSP00000383144 |
| 379 | YTDC2_HUMAN | Q9H6S0 | GIPNDSSDSEMEDK | 325 | 1 | YTH domain-containing |
| | | | | | | protein 2 |
| | YTHD1_HUMAN | Q9BYJ9 | | 165 | 1 | YTH domain family protein 1 |
| | YTHD1_HUMAN | Q9BYJ9 | | 165 | 1 | YTH domain family protein 1 |
| | YTHD1_HUMAN | Q9BYJ9 | | 165 | 1 | YTH domain family protein 1 |
| 380 | YTHD2_HUMAN | Q9Y5A9 | GNGVGQSQAGSG | 368 | 1 | YTH domain family protein 2 |
| | | | STPSEPHPVLEKLR | | | |
| 381 | YTHD2_HUMAN | Q9Y5A9 | GQSAFANETLNK | 167 | 1 | YTH domain family protein 2 |
| 382 | YTHD2_HUMAN | Q9Y5A9 | GQSAFANETLNK | 167 | 1 | YTH domain family protein 2 |
| | | | APGMNTIDQGMA | | | |
| | | | ALK | | | |
| 383 | YTHD2_HUMAN | Q9Y5A9 | GQSAFANETLNK | 167 | 1 | YTH domain family protein 2 |
| | | | APGMNTIDQGMA | | | |
| | | | ALKLGSTEVASN | | | |
| | | | VPK | | | |
| | YTHD2_HUMAN | Q9Y5A9 | | 368 | 1 | YTH domain family protein 2 |
| | YTHD3_HUMAN | Q7Z739 | | 169 | 1 | YTH domain family protein 3 |
| | YTHD3_HUMAN | Q7Z739 | | 169 | 1 | YTH domain family protein 3 |
| | ZAP70_HUMAN | P43403 | | 291 | 1 | Tyrosine-protein kinase |
| | | | | | | ZAP-70 |
| | ZBT34_HUMAN | Q8NCN2 | | 140 | 1 | Zinc finger and BTB |
| | | | | | | domain-containing protein |
| | | | | | | 34 |
| 384 | ZBT44_HUMAN | Q8NCP5 | GSISPVSSECSVVER | 158 | 1 | Zinc finger and BTB |
| | | | | | | domain-containing protein |
| | | | | | | 44 |
| | ZC11A_HUMAN | O75152 | | 349 | 1 | Zinc finger CCCH domain- |
| | | | | | | containing protein 11A |
| | ZC11A_HUMAN | O75152 | | 531 | 1 | Zinc finger CCCH domain- |
| | | | | | | containing protein 11A |
| | ZC3H4_HUMAN | Q9UPT8 | | 68 | 1 | Zinc finger CCCH domain- |
| | | | | | | containing protein 4 |
| | ZC3H4_HUMAN | Q9UPT8 | | 742 | 1 | Zinc finger CCCH domain- |
| | | | | | | containing protein 4 |
| | ZC3HD_HUMAN | Q5T200 | | 160 | 1 | Zinc finger CCCH domain- |
| | | | | | | containing protein 13 |
| | ZC3HD_HUMAN | Q5T200 | | 160 | 1 | Zinc finger CCCH domain- |
| | | | | | | containing protein 13 |
| 385 | ZC3HE_HUMAN | Q6PJT7 | GVPSPPGYMSDQ | 524 | 1 | Zinc finger CCCH domain- |
| | | | EEDMCFEGMKPV | | | containing protein 14 |
| | | | NQTAASNKGLR | | | |
| 386 | ZCCHV_HUMAN | Q7Z2W4 | GVATDITSTR | 434 | 1 | Zinc finger CCCH-type |
| | | | | | | antiviral protein 1 |
| 387 | ZCCHV_HUMAN | Q7Z2W4 | SLSDVTSTTSSR | 492 | 1 | Zinc finger CCCH-type |
| | | | | | | antiviral protein 1 |
| 388 | ZCH18_HUMAN | Q86VM9 | TVLEPYADPYYD | 362 | 1 | Zinc finger CCCH domain- |
| | | | YEIER | | | containing protein 18 |
| | ZCHC2_HUMAN | Q9C0B9 | | 235 | 1 | Zinc finger CCHC domain- |
| | | | | | | containing protein 2 |
| 389 | ZCHC8_HUMAN | Q6NZY4 | GETEVGEIQQNK | 344 | 1 | Zinc finger CCHC domain- |
| | | | | | | containing protein 8 |
| 390 | ZEB1_HUMAN | P37275 | AADCEGVPEDDL | 50 | 1 | Zinc finger E-box-binding |
| | | | PTDQTVLPGR | | | homeobox 1 |
| | ZF161_HUMAN | O43829 | | 244 | 1 | Zinc finger protein 161 |
| | | | | | | homolog |
| | ZF161_HUMAN | O43829 | | 244 | 1 | Zinc finger protein 161 |
| | | | | | | homolog |
| | ZFAN6_HUMAN | Q6FIF0 | | 107 | 1 | AN1-type zinc finger protein 6 |
| | ZFAN6_HUMAN | Q6FIF0 | | 127 | 1 | AN1-type zinc finger protein 6 |
| | ZFPL1_HUMAN | O95159 | | 172 | 1 | Zinc finger protein-like 1 |
| 391 | ZFX_HUMAN | P17010 | GTCPEVIK | 245 | 1 | Zinc finger X-chromosomal |
| | | | | | | protein |
| 392 | ZFX_HUMAN | P17010 | GTCPEVIKVYIFK | 245 | 1 | Zinc finger X-chromosomal |
| | | | | | | protein |
| | ZFY16_HUMAN | Q7Z3T8 | | 535 | 1 | Zinc finger FYVE domain- |
| | | | | | | containing protein 16 |
| | ZFY16_HUMAN | Q7Z3T8 | | 108 | 1 | Zinc finger FYVE domain- |
| | | | | | | containing protein 16 |
| | ZFY16_HUMAN | Q7Z3T8 | | 284 | 1 | Zinc finger FYVE domain- |
| | | | | | | containing protein 16 |
| 393 | ZMYM3_HUMAN | Q14202 | STESIPVSDEDSD | 256 | 1 | Zinc finger MYM-type |
| | | | AMVDDPNDEDFV | | | protein 3 |
| | | | PFRPR | | | |
| | ZMYM4_HUMAN | Q5VZL5 | | 929 | 1 | Zinc finger MYM-type |
| | | | | | | protein 4 |
| | ZN143_HUMAN | P52747 | | 183 | 1 | Zinc finger protein 143 |
| | ZN143_HUMAN | P52747 | | 152 | 1 | Zinc finger protein 143 |
| | ZN200_HUMAN | P98182 | | 189 | 1 | Zinc finger protein 200 |
| | ZN264_HUMAN | O43296 | | 160 | 1 | Zinc finger protein 264 |
| | ZN277_HUMAN | Q9NRM2 | | 7 | 1 | Zinc finger protein 277 |
| | ZN346_HUMAN | Q9UL40 | | 14 | 1 | Zinc finger protein 346 |
| 394 | ZN644_HUMAN | Q9H582 | SFGSPLGLDKR | 616 | 1 | Zinc finger protein 644 |
| 395 | ZN644_HUMAN | Q9H582 | SFGSPLGLDKRK | 616 | 1 | Zinc finger protein 644 |
| | ZN646_HUMAN | O15015 | | 1006 | 1 | Zinc finger protein 646 |
| | ZN787_HUMAN | Q6DD87 | | 231 | 1 | Zinc finger protein 787 |
| 396 | ZN828_HUMAN | Q96JM3 | AIDDQKCDILVQE | 586 | 1 | Zinc finger protein 828 |
| | | | ELLASPK | | | |
| 397 | ZN828_HUMAN | Q96JM3 | AIDDQKCDILVQE | 586 | 1 | Zinc finger protein 828 |
| | | | ELLASPKK | | | |
| 398 | ZNF24_HUMAN | P17028 | SILIIPTPDEEEKILR | 10 | 1 | Zinc finger protein 24 |
| 399 | ZNF24_HUMAN | P17028 | SILIIPTPDEEEK | 10 | 1 | Zinc finger protein 24 |
| | ZNF76_HUMAN | P36508 | | 14 | 1 | Zinc finger protein 76 |
| | ZNHI2_HUMAN | Q9UHR6 | | 145 | 1 | Zinc finger HIT domain- |
| | | | | | | containing protein 2 |
| | ZNHI2_HUMAN | Q9UHR6 | | 145 | 1 | Zinc finger HIT domain- |
| | | | | | | containing protein 2 |
| | ZYX_HUMAN | Q15942 | | 150 | 1 | Zyxin |
| 400 | ZYX_HUMAN | Q15942 | SLSSLLDDMTK | 150 | 1 | Zyxin |
| 401 | ZYX_HUMAN | Q15942 | SLSSLLDDMTKN | 150 | 1 | Zyxin |
| | | | DPFKAR | | | |

TABLE 2
Mass spectrometry statistics for identified
caspase-derived peptides.

| Swiss-Prot | | | | | |
|---|---|---|---|---|---|
| acc # | m/z | z | Error ppm | score | E value |
| Q13362 | 695.0352 | 3 | −8.3 | 60.6 | 2.80E−06 |
| P29372 | 833.0408 | 3 | −3.3 | 44.3 | 6.30E−05 |
| P11171 | 756.4105 | 4 | 21 | 51.9 | 4.30E−08 |
| P11171 | 663.9429 | 5 | −2.3 | 62.5 | 2.50E−09 |
| Q13541 | 938.7966 | 3 | 6.1 | 55.9 | 2.30E−07 |
| Q13542 | 952.7778 | 3 | −2.4 | 55 | 1.80E−08 |
| Q6S8J3 | 737.909 | 2 | −1.2 | 24.9 | 0.0013 |
| A5A3E0 | | | | | |
| P62736 | | | | | |
| Q562R1 | | | | | |
| P60709 | | | | | |
| P68032 | | | | | |
| P63261 | | | | | |
| P63267 | | | | | |
| Q9BYX7 | | | | | |
| P68133 | | | | | |
| Q6S8J3 | 586.3246 | 2 | −0.82 | 30.7 | 2.60E−04 |
| A5A3E0 | | | | | |
| P62736 | | | | | |
| Q562 | | | | | |
| P60709 | | | | | |
| P68032 | | | | | |
| P63261 | | | | | |
| P63267 | | | | | |
| Q9BYX7 | | | | | |
| P68133 | | | | | |
| Q6S8J3 | 600.9496 | 3 | 1.2 | 43.4 | 9.70E−06 |
| P60709 | | | | | |
| P63261 | | | | | |
| Q9BTE6 | 682.349 | 3 | −6.1 | 48.5 | 4.10E−04 |
| P00519 | 640.3181 | 3 | −5.7 | 56 | 1.70E−05 |
| O14639 | 657.989 | 3 | 5.1 | 45.6 | 8.30E−06 |
| Q96P50 | 836.3983 | 2 | −14 | 42.9 | 5.50E−07 |
| Q9UKV3 | 1144.8881 | 3 | 0.63 | 24.3 | 0.092 |
| Q9UKV3 | 541.5953 | 3 | 3.6 | 39.8 | 2.20E−05 |
| Q9UKV3 | 378.2309 | 3 | 27 | 28.4 | 0.0014 |
| Q9UKV3 | 627.2832 | 2 | 4.8 | 34.5 | 0.009 |
| P21399 | 812.425 | 2 | −1.4 | 23.4 | 4.10E−04 |
| O95573 | 612.8007 | 2 | −8.2 | 39.5 | 0.014 |
| O60488 | | | | | |
| P62736 | 662.8276 | 2 | −0.9 | 34.1 | 3.00E−06 |
| P60709 | | | | | |
| P68032 | | | | | |
| P63261 | | | | | |
| P63267 | | | | | |
| P68133 | | | | | |
| P62736 | 584.7781 | 2 | 0.66 | 38.2 | 5.90E−06 |
| P60709 | | | | | |
| P68032 | | | | | |
| P63261 | | | | | |
| P63267 | | | | | |
| P68133 | | | | | |
| P60709 | 638.5862 | 4 | −2.9 | 42.2 | 1.80E−06 |
| P63261 | | | | | |
| P60709 | 615.0721 | 4 | −11 | 40.9 | 4.10E−07 |
| P63261 | | | | | |
| P12814 | 1108.499 | 3 | 0.83 | 30.6 | 2.10E−04 |
| P12814 | 1103.1478 | 3 | −17 | 22.4 | 0.011 |
| P12814 | 667.8556 | 2 | 3.7 | 41.5 | 0.096 |
| P35609 | | | | | |
| Q08043 | | | | | |
| O43707 | | | | | |
| P35611 | 996.1574 | 3 | −18 | 33.4 | 4.90E−05 |
| Q6ZN18 | 396.7168 | 2 | 6.6 | 28.8 | 0.023 |
| Q96SZ5 | 1006.9929 | 2 | 1.9 | 22.8 | 0.0013 |
| Q8N4X5 | 770.9177 | 2 | −5.7 | 42.4 | 2.80E−04 |
| Q8N4X5 | 604.6598 | 3 | −0.055 | 39.4 | 0.019 |
| Q8N4X5 | 415.2028 | 3 | −1.2 | 28.2 | 0.02 |
| Q6ULP2 | 401.8817 | 3 | 19 | 29.6 | 0.085 |
| Q8N302 | 432.7184 | 2 | 9.9 | 28 | 0.0053 |
| Q8N302 | 439.4802 | 4 | 9.6 | 32.2 | 3.70E−04 |
| Q8N302 | 534.2534 | 3 | −9.1 | 32.4 | 0.0061 |
| Q09666 | 794.9363 | 2 | −2.5 | 23.4 | 9.00E−04 |
| Q09666 | 514.2765 | 2 | 3.8 | 31.7 | 0.0041 |
| Q09666 | 416.5694 | 3 | −4.7 | 31.8 | 0.005 |
| Q09666 | 598.7812 | 2 | −20 | 38.5 | 0.068 |
| Q09666 | 463.9146 | 3 | −5.8 | 44.8 | 0.013 |
| Q09666 | 481.7599 | 2 | −19 | 29.6 | 0.052 |
| Q09666 | 496.2643 | 2 | −25 | 24.4 | 0.058 |
| Q09666 | 388.9439 | 4 | −0.37 | 39.1 | 3.00E−04 |
| Q09666 | 519.9274 | 3 | −1.2 | 48.4 | 5.60E−04 |
| Q09666 | 397.2338 | 3 | −5.9 | 29.2 | 0.0027 |
| Q09666 | 446.888 | 3 | −7 | 30.8 | 0.025 |
| Q09666 | 439.2175 | 3 | −3.9 | 32 | 0.0024 |
| Q09666 | 596.7931 | 2 | −4.6 | 39.1 | 0.055 |
| Q09666 | 514.9217 | 3 | −13 | 40.8 | 0.0015 |
| Q09666 | 583.2912 | 2 | 1.3 | 40.7 | 0.041 |
| Q09666 | 583.2881 | 2 | −4 | 37.2 | 0.048 |
| O95433 | 788.8887 | 2 | −9.6 | 37.5 | 6.50E−05 |
| O95433 | 547.0416 | 4 | 5.6 | 30.2 | 2.80E−04 |
| O95433 | 593.2786 | 3 | 9.7 | 43.6 | 8.50E−05 |
| Q8WYP5 | 960.9803 | 2 | −0.86 | 36.5 | 5.00E−06 |
| Q9Y4K1 | 571.6341 | 3 | 3.7 | 57.2 | 3.60E−06 |
| Q9Y4K1 | 904.4601 | 3 | −1.9 | 26.4 | 0.026 |
| Q02952 | 596.9709 | 3 | −1.9 | 35.8 | 0.02 |
| Q9Y2D5 | 530.78 | 2 | −9.9 | 22.1 | 0.027 |
| Q99996 | 476.256 | 2 | 12 | 34.5 | 4.80E−04 |
| Q99996 | 468.2558 | 2 | 6.9 | 35.5 | 0.0067 |
| Q7Z591 | 438.5817 | 3 | 10 | 42.8 | 2.70E−05 |
| Q12802 | 737.6817 | 3 | −4.9 | 46.5 | 1.60E−04 |
| Q12802 | 772.363 | 4 | −3.1 | 44.8 | 4.50E−05 |
| Q12802 | 562.265 | 3 | −4.8 | 35.9 | 3.00E−04 |
| Q12802 | 474.2589 | 2 | 6.4 | 34.5 | 0.0039 |
| Q12802 | 917.9474 | 2 | −7 | 52.6 | 1.00E−05 |
| Q12802 | 848.4239 | 2 | 2 | 29.9 | 0.052 |
| Q9ULX6 | 707.3197 | 2 | 5.1 | 40.1 | 3.20E−05 |
| Q8TCU4 | 716.8455 | 2 | −0.84 | 39.6 | 6.80E−05 |
| Q8TCU4 | 530.2837 | 4 | −2.8 | 30 | 0.005 |
| Q8TCU4 | 556.8022 | 2 | 2.3 | 25.9 | 3.30E−04 |
| Q9HCF4 | 527.7967 | 2 | −2.9 | 36.4 | 4.40E−04 |
| Q9HCF4 | 684.3854 | 2 | −0.51 | 23.9 | 0.0017 |
| Q9HCF4 | 878.4746 | 2 | −0.13 | 30.8 | 6.10E−06 |
| Q01432 | 1012.9898 | 2 | −14 | 42 | 6.50E−05 |
| P53582 | 608.6207 | 3 | 1.5 | 49.8 | 7.80E−07 |
| Q8IWZ3 | 632.3286 | 5 | 14 | 26.7 | 0.007 |
| Q8IWZ3 | 933.4111 | 2 | −19 | 22 | 2.60E−04 |
| Q68DC2 | 518.28 | 3 | −7.5 | 43 | 0.0026 |
| Q92625 | 440.5292 | 3 | −5.1 | 36 | 7.90E−04 |
| P07355 | 494.2418 | 3 | 2.7 | 38.9 | 1.10E−04 |
| A6NMY6 | | | | | |
| O43747 | 867.4424 | 3 | −4.5 | 51.8 | 5.70E−08 |
| O75843 | 833.4361 | 5 | −5.6 | 34.6 | 0.0022 |
| O94973 | 679.9964 | 3 | −6.4 | 49.5 | 3.40E−06 |
| Q13367 | 608.3474 | 4 | −6.1 | 22.8 | 0.041 |
| Q13367 | 910.0015 | 2 | −0.16 | 39.4 | 7.10E−05 |
| Q92870 | 792.7011 | 3 | −1.8 | 43 | 2.10E−04 |
| P25054 | 957.4693 | 3 | −11 | 52.5 | 4.50E−09 |
| Q9HDC9 | 475.7311 | 2 | −2.9 | 33 | 0.084 |
| Q7Z2E3 | 891.0972 | 3 | −8.3 | 50 | 3.20E−06 |
| Q3SXY8 | 1169.6076 | 3 | 2 | 31.7 | 0.0066 |
| P25098 | 491.9294 | 3 | 5.4 | 36 | 0.0013 |
| P25098 | 619.6332 | 3 | −3.3 | 31.5 | 1.70E−04 |
| P35626 | | | | | |
| P25098 | 779.8454 | 2 | −4.6 | 32.2 | 8.90E−07 |
| P35626 | | | | | |
| Q92888 | 398.8444 | 3 | −47 | 26.6 | 0.0068 |
| Q92974 | 711.8592 | 2 | 4.6 | 32.7 | 1.10E−04 |
| O15013 | 1053.8212 | 3 | −24 | 23.4 | 2.10E−04 |
| O14497 | 830.8775 | 2 | −0.32 | 22.6 | 3.00E−05 |
| O14497 | 822.8813 | 2 | 1.2 | 29.3 | 1.50E−06 |
| O14497 | 774.0026 | 3 | −6.1 | 65.8 | 1.40E−07 |
| P29374 | 796.8669 | 2 | 2.8 | 36.1 | 2.90E−06 |
| Q4LE39 | 1357.619 | 3 | 2.2 | 28.9 | 2.60E−05 |
| Q68CP9 | 710.3607 | 4 | −2.5 | 34.7 | 2.40E−05 |
| Q68CP9 | 652.071 | 4 | −11 | 23.6 | 0.0054 |
| Q8N2F6 | 915.4348 | 2 | 3.9 | 49.2 | 2.40E−04 |
| Q6NXE6 | 969.2285 | 4 | −0.03 | 23.1 | 0.046 |
| Q6NXE6 | 965.2247 | 4 | −5.3 | 42.8 | 1.40E−06 |
| P27540 | 621.6545 | 3 | 17 | 41.6 | 4.50E−04 |
| Q9UBL0 | 663.9974 | 3 | −13 | 43 | 3.50E−06 |
| P61160 | 567.7937 | 4 | −2.9 | 25.9 | 0.0085 |
| P61158 | 694.0126 | 3 | −2 | 34.8 | 1.00E−03 |
| O15511 | 952.3894 | 2 | −20 | 61.5 | 3.00E−08 |
| O15511 | 837.8722 | 2 | −0.95 | 43.1 | 3.90E−08 |
| Q9BXP5 | 406.8858 | 3 | 8.5 | 32.4 | 4.10E−04 |
| Q8WXK3 | 817.952 | 2 | −0.3 | 26.3 | 6.10E−04 |
| Q8N9N2 | 1067.091 | 3 | −15 | 36.1 | 8.00E−07 |
| Q9H1I8 | 709.3174 | 3 | 5.1 | 64.5 | 6.50E−08 |
| Q9BVC5 | 476.7547 | 2 | 7.9 | 27.4 | 0.016 |
| Q13625 | 884.4693 | 3 | −0.001 | 28.5 | 0.0026 |
| Q96QE3 | 990.488 | 2 | 1.2 | 22 | 0.003 |
| Q9ULI0 | 761.9025 | 2 | 0.34 | 27.9 | 2.30E−04 |
| P18846 | 378.2114 | 4 | −17 | 28.1 | 0.0012 |
| P18848 | 866.4062 | 3 | −5.7 | 45.6 | 2.90E−06 |
| P17544 | 742.397 | 2 | −17 | 42 | 0.0031 |
| P17544 | 624.6849 | 3 | −10 | 27.6 | 0.01 |
| Q9NT62 | 706.7647 | 5 | 0.57 | 29.7 | 0.0033 |
| Q9NT62 | 790.3876 | 3 | 3.1 | 58.8 | 1.30E−06 |
| Q9Y4P1 | 687.847 | 2 | −2.3 | 43.1 | 0.0069 |
| P46100 | 689.0362 | 3 | 0.84 | 34 | 5.70E−04 |
| P0C7T5 | 631.8268 | 2 | −1.9 | 27 | 7.30E−04 |
| Q8WWM7 | 878.4225 | 2 | −5.3 | 55.1 | 2.70E−07 |
| Q8WWM7 | 870.4253 | 2 | −5 | 50.7 | 3.20E−05 |
| Q99700 | 1189.2343 | 3 | 4.8 | 26.6 | 0.0068 |
| P54252 | 839.8587 | 2 | −7.4 | 40.4 | 3.10E−04 |
| Q9UPN4 | 971.4637 | 3 | −11 | 30.2 | 6.50E−06 |
| Q9Y520 | 832.8634 | 2 | −8.3 | 40.2 | 2.20E−06 |
| Q9Y520 | 716.0153 | 3 | 0.7 | 49.7 | 5.50E−06 |
| Q92560 | 759.3601 | 3 | −2.9 | 31.8 | 0.0062 |
| P51572 | 479.5827 | 3 | −3.1 | 42 | 6.90E−05 |
| P51572 | 552.5266 | 4 | −4.3 | 52.9 | 2.30E−08 |
| P80723 | 660.9797 | 3 | 5.1 | 49.9 | 1.20E−05 |
| P80723 | 494.9073 | 3 | 0.21 | 43.6 | 3.50E−04 |
| P46379 | 1087.21 | 3 | −7 | 37.4 | 3.70E−05 |
| P46379 | 1129.9151 | 3 | −0.73 | 37.4 | 6.60E−06 |
| Q9NRL2 | 678.316 | 3 | −11 | 24.9 | 0.0029 |
| Q6ZUJ8 | 750.3245 | 2 | 2.2 | 57.3 | 4.20E−04 |
| Q6ZUJ8 | 721.6722 | 3 | 2.1 | 49.5 | 1.40E−05 |
| Q9NYF8 | 528.2364 | 2 | 1.5 | 29.8 | 0.021 |
| Q9NYF8 | 437.5387 | 3 | 9 | 32.9 | 3.40E−04 |
| P11274 | 682.3057 | 2 | −6.1 | 24.6 | 6.20E−04 |
| A6H8Y1 | 803.4489 | 2 | −3.3 | 29.3 | 2.90E−04 |
| P55957 | 581.2888 | 2 | −3.7 | 39.6 | 0.0038 |
| Q5TH69 | 385.5289 | 3 | 15 | 28.2 | 0.0034 |
| O00499 | 542.2935 | 2 | 0.33 | 31.9 | 8.20E−04 |
| Q9NR09 | 398.861 | 3 | −5.4 | 26.8 | 0.025 |
| Q6QNY0 | 878.9029 | 2 | −2.2 | 40.9 | 0.0018 |
| Q8WV28 | 1031.4701 | 3 | −1.6 | 34.3 | 4.70E−05 |
| Q12982 | 933.0836 | 3 | −4 | 37.9 | 6.20E−05 |
| Q12830 | 613.3208 | 2 | 2.6 | 44.1 | 0.027 |
| O95696 | 624.8586 | 2 | 21 | 25 | 0.0048 |
| O60885 | 424.5384 | 3 | −0.75 | 32.9 | 0.0026 |
| Q9H0E9 | 597.9905 | 3 | 4.6 | 52 | 8.20E−05 |
| Q96RE7 | 420.8942 | 3 | 10 | 29.4 | 0.037 |
| O43683 | 463.2503 | 2 | 13 | 28.4 | 0.0023 |
| Q43683 | 835.4028 | 2 | −3.2 | 28.7 | 4.70E−06 |
| Q9BRD0 | 740.8795 | 2 | −19 | 46 | 0.0071 |
| Q96L14 | 832.4292 | 2 | 0.79 | 24.5 | 3.60E−04 |
| Q07021 | 904.0546 | 3 | −3 | 48.5 | 4.20E−09 |
| O14523 | 567.6593 | 3 | −1.2 | 49.4 | 7.10E−06 |
| Q6P1N0 | 1118.5371 | 3 | −15 | 52.9 | 4.20E−08 |
| Q6P1N0 | 1113.2059 | 3 | −14 | 52 | 2.70E−08 |
| Q5T0F9 | 550.814 | 2 | −9.3 | 39.7 | 0.0015 |
| Q5T8I9 | 516.7592 | 2 | 0.29 | 28.6 | 0.031 |
| Q5T8I9 | 619.8312 | 4 | 1.9 | 30.4 | 0.0013 |
| Q5T3J3 | 741.8712 | 2 | −1.3 | 22.9 | 7.10E−05 |
| Q5T3J3 | 1040.5147 | 2 | 4.5 | 26.6 | 1.70E−04 |
| Q96BR5 | 721.3472 | 2 | −3.2 | 27.7 | 1.10E−04 |
| Q7L4P6 | 579.2901 | 3 | 3.5 | 31.4 | 2.90E−04 |
| Q5SV97 | 881.8479 | 5 | −5.2 | 23 | 0.028 |
| Q68CQ1 | 552.8186 | 2 | −10 | 34.7 | 0.071 |
| Q08AD1 | 578.2992 | 2 | 6.8 | 36.9 | 0.016 |
| Q9BTV7 | 579.3169 | 4 | 22 | 36.9 | 1.50E−04 |
| Q9P1Z2 | 674.3779 | 2 | −2.5 | 47.5 | 2.10E−05 |
| P19022 | 547.86 | 2 | −2.8 | 22.4 | 0.0049 |
| P19022 | 368.5621 | 3 | 13 | 24.2 | 0.0094 |
| Q13111 | 661.5167 | 5 | 16 | 51 | 9.80E−09 |
| Q13111 | 449.2444 | 2 | 3 | 25.6 | 0.021 |
| Q13111 | 919.9718 | 2 | 0.46 | 35.5 | 1.30E−06 |
| P27797 | 631.8193 | 4 | −2.6 | 51.1 | 1.10E−06 |
| P27797 | 911.7541 | 3 | −7.3 | 34.9 | 5.90E−04 |
| P27797 | 870.0258 | 3 | −6.1 | 54.5 | 1.90E−07 |
| Q8NCB2 | 865.9321 | 2 | 0.86 | 25.9 | 2.90E−05 |
| P49069 | 642.0082 | 3 | −0.29 | 26 | 0.0051 |
| P49069 | 644.9868 | 3 | −3.2 | 62.1 | 2.40E−06 |
| P49069 | 747.6898 | 3 | −14 | 48.1 | 1.50E−05 |
| Q5T5Y3 | 487.9148 | 3 | −0.65 | 28.9 | 5.30E−04 |
| Q5T5Y3 | 686.1413 | 5 | 2.1 | 27.1 | 9.90E−04 |
| Q5T5Y3 | 779.045 | 3 | −1.6 | 52.6 | 1.10E−05 |
| Q14444 | 642.6701 | 3 | −0.51 | 51.8 | 2.30E−07 |
| P47756 | 418.2392 | 3 | 11 | 33.5 | 5.90E−04 |
| O15234 | 513.2743 | 3 | 4.9 | 30.6 | 6.70E−05 |
| Q8NG31 | 810.8847 | 2 | −4 | 32.6 | 1.20E−06 |
| P42574 | 667.3261 | 2 | 13 | 41 | 0.0012 |
| P42574 | 500.8599 | 3 | −5.2 | 39.2 | 1.30E−05 |
| P42574 | 495.5289 | 3 | −3.9 | 30.9 | 1.00E−04 |
| P55210 | 671.3188 | 2 | −6.7 | 54.1 | 3.20E−05 |
| Q13948 | 525.3142 | 3 | 5.1 | 29.9 | 4.40E−04 |
| P39880 | | | | | |
| P07858 | 456.5788 | 3 | −7.3 | 36.9 | 0.036 |
| Q9H6R7 | 954.0162 | 2 | 0.98 | 46.1 | 5.70E−07 |
| P22681 | 689.8437 | 4 | −4 | 37 | 3.20E−06 |
| Q9BRT8 | 479.7709 | 2 | 3 | 32.6 | 0.072 |
| Q8IUF1 | | | | | |
| Q5JTY5 | | | | | |
| Q5RIA9 | | | | | |
| Q4V339 | | | | | |
| A6NM15 | | | | | |
| Q96G28 | 559.2568 | 3 | 4.8 | 27.2 | 0.0029 |
| Q96G28 | 553.9217 | 3 | −1.5 | 29.9 | 4.00E−04 |
| Q96G28 | 467.5685 | 3 | 13 | 24.4 | 0.0025 |
| Q96G28 | 527.9086 | 3 | 0.77 | 40.9 | 3.70E−05 |
| Q96CT7 | 546.7925 | 4 | 20 | 27.8 | 0.0032 |
| O60293 | 563.6239 | 3 | 0.51 | 42.9 | 2.20E−05 |
| Q9NV96 | 577.7865 | 2 | 2.9 | 52.4 | 2.10E−04 |
| Q96MW1 | 736.8328 | 2 | 2 | 51.9 | 5.60E−05 |
| Q9Y3C0 | 776.4054 | 2 | −3.3 | 47.6 | 0.007 |
| Q7Z6B0 | 791.9203 | 2 | −1.9 | 38.8 | 0.005 |
| Q96F63 | 734.3549 | 3 | −5.2 | 26.5 | 0.0023 |
| Q9Y3X0 | 616.3044 | 3 | −1.6 | 38.3 | 6.50E−06 |
| Q9Y3X0 | 507.7253 | 4 | −30 | 33 | 8.60E−05 |
| O60583 | 768.8993 | 2 | −0.24 | 34.5 | 4.40E−05 |
| O60583 | 1060.8711 | 3 | −1.4 | 31.1 | 0.0011 |
| P21127 | 807.4354 | 2 | −9.3 | 54.5 | 1.70E−07 |
| P21127 | 737.069 | 3 | −8.6 | 47.5 | 1.10E−07 |
| Q14004 | 628.3353 | 2 | −0.65 | 30.1 | 5.10E−05 |
| P30260 | 1014.5668 | 2 | 2 | 29.5 | 1.10E−04 |
| P30260 | 762.4204 | 2 | 1.3 | 51.1 | 0.001 |
| Q99459 | 571.2824 | 2 | −5.4 | 37.5 | 0.0019 |
| Q9BWT1 | 657.8024 | 2 | −7.4 | 37.5 | 5.80E−05 |
| Q9UKY7 | 725.3042 | 2 | −3.5 | 33.7 | 9.40E−05 |
| Q9Y232 | 1439.7215 | 3 | −0.52 | 42.5 | 3.20E−06 |
| Q49AR2 | 586.9478 | 3 | 8.5 | 41.8 | 2.40E−05 |
| O94986 | 626.0437 | 4 | −6.9 | 36 | 1.90E−04 |
| Q5SW79 | 870.4244 | 3 | −12 | 50.5 | 2.10E−06 |
| Q5SW79 | 1117.558 | 3 | 1.1 | 26.3 | 0.0077 |
| Q5SW79 | 559.9728 | 3 | 1.3 | 24.6 | 0.015 |
| Q03701 | 869.0709 | 3 | 0.25 | 24.2 | 0.001 |
| Q03701 | 445.2475 | 2 | 7.5 | 22.2 | 0.023 |
| Q03701 | 608.79 | 2 | −1.6 | 40 | 0.0048 |
| Q6NXR4 | 1017.8124 | 3 | 1.6 | 25 | 0.016 |
| Q6P1X6 | 774.3731 | 2 | −3.8 | 29.5 | 2.90E−06 |
| P10809 | 698.8351 | 2 | 2.1 | 38 | 5.30E−04 |
| P10809 | 538.3097 | 2 | 1.4 | 33.2 | 0.0055 |
| P10809 | 594.2953 | 2 | −5.8 | 39.6 | 0.0068 |
| P10809 | 528.3024 | 4 | 0.58 | 23.6 | 0.02 |
| Q12873 | 1130.1293 | 3 | −7.1 | 40.5 | 5.50E−07 |
| Q14839 | | | | | |
| Q8TDI0 | | | | | |
| Q14839 | 568.7467 | 4 | 0.63 | 38.4 | 3.10E−06 |
| Q14839 | 686.0461 | 4 | −24 | 34.5 | 3.20E−05 |
| Q9P2D1 | 777.361 | 3 | 12 | 51 | 1.80E−06 |
| Q9BY43 | 618.8337 | 2 | 1.5 | 34.6 | 0.0014 |
| Q9H444 | | | | | |
| Q96CF2 | | | | | |
| Q9NRY2 | 546.9371 | 3 | −0.91 | 40.1 | 3.80E−04 |
| Q5VWN6 | 526.2781 | 2 | 11 | 40.6 | 3.00E−04 |
| Q86WR7 | 931.142 | 3 | 7.8 | 55.6 | 3.50E−09 |
| Q6IAA8 | 598.2323 | 3 | −4.3 | 29.2 | 0.0064 |
| Q6IAA8 | 592.907 | 3 | 6.4 | 23.6 | 0.041 |
| Q9HCM1 | 701.8622 | 2 | −0.14 | 27.8 | 5.00E−05 |
| Q9HCM1 | 741.8753 | 2 | −2.8 | 43.8 | 0.064 |
| Q96C57 | 688.8449 | 2 | 1.5 | 26.1 | 3.70E−04 |
| Q96C57 | 832.9297 | 2 | −0.19 | 27.4 | 7.50E−04 |
| Q96C57 | 824.9339 | 2 | 1.8 | 31.3 | 8.30E−06 |
| Q96C57 | 953.0076 | 2 | −1.6 | 23.2 | 3.70E−04 |
| Q7Z460 | 612.289 | 2 | 4.7 | 39.3 | 0.0017 |
| Q7Z460 | 684.6447 | 3 | −8.9 | 39.5 | 7.40E−05 |
| P09496 | 1039.7784 | 3 | −5.5 | 63.1 | 4.30E−10 |
| P09496 | 1034.4414 | 3 | −11 | 59.2 | 1.40E−10 |
| P09496 | 680.3305 | 2 | −0.11 | 40.2 | 6.10E−04 |
| O00299 | 917.4094 | 3 | −18 | 29.1 | 2.90E−04 |
| P30622 | 487.59 | 3 | 4.3 | 24 | 0.038 |
| Q9HAW4 | 940.0497 | 2 | 1.2 | 32.6 | 5.20E−06 |
| Q15003 | 534.2601 | 2 | 8.6 | 40.5 | 8.00E−04 |
| Q15003 | 708.3025 | 4 | 2.1 | 29.7 | 1.50E−05 |
| Q15003 | 461.5532 | 3 | 0.11 | 36.7 | 7.50E−06 |
| Q15003 | 394.2029 | 3 | 17 | 25.1 | 0.02 |
| Q15003 | 624.776 | 2 | 2.7 | 34.2 | 2.60E−05 |
| Q6IBW4 | 578.9792 | 3 | 1.3 | 35.7 | 6.90E−04 |
| P12111 | 1063.8161 | 3 | −21 | 30.7 | 0.0079 |
| Q53SF7 | 889.4591 | 2 | 3.8 | 44 | 6.80E−04 |
| P53621 | 914.9333 | 2 | −0.38 | 53.3 | 5.70E−07 |
| P53621 | 650.848 | 2 | 8 | 29.3 | 0.0038 |
| P35606 | 603.5815 | 4 | 20 | 35 | 0.0012 |
| P31146 | 455.7318 | 2 | −12 | 23.2 | 0.033 |
| Q1ED39 | 484.2491 | 4 | 5.1 | 25.1 | 0.02 |
| Q7Z7A1 | 838.9225 | 2 | −2 | 29.1 | 4.20E−05 |
| Q7Z7A1 | 482.9271 | 3 | −2.3 | 30.2 | 0.017 |
| Q6FI81 | 556.5493 | 4 | 11 | 29.9 | 2.10E−04 |
| Q99829 | 494.2491 | 2 | −9.9 | 31.4 | 0.011 |
| O75131 | 602.3151 | 2 | 1.1 | 23.1 | 6.90E−04 |
| Q16630 | 724.8665 | 2 | −0.77 | 47.3 | 0.027 |
| Q8N684 | 721.3527 | 3 | 0.039 | 26.5 | 8.10E−04 |
| Q8N684 | 682.6449 | 3 | −4.8 | 44.1 | 2.70E−06 |
| Q8N684 | 770.3681 | 2 | 0.24 | 42.4 | 0.0031 |
| Q6JBY9 | 908.7383 | 3 | −4.8 | 61.4 | 1.10E−08 |
| Q96N21 | 1176.6536 | 2 | 3.3 | 24.7 | 2.30E−04 |
| Q53F19 | 666.9666 | 3 | −2.1 | 24.7 | 0.096 |
| Q53F19 | 744.8885 | 2 | −14 | 46.5 | 1.80E−04 |
| Q96B23 | 779.3919 | 3 | −6.8 | 31.3 | 5.20E−04 |
| Q96B23 | 727.367 | 3 | 4.8 | 38.3 | 1.30E−04 |
| Q96B23 | 779.3968 | 3 | −0.56 | 28.3 | 0.0065 |
| Q96B23 | 722.0345 | 3 | 3.7 | 40.8 | 8.90E−05 |
| Q96B23 | 774.055 | 3 | −14 | 40.5 | 3.00E−04 |
| P16220 | 961.8356 | 3 | −14 | 49.5 | 4.30E−07 |
| P16220 | 425.2108 | 2 | −20 | 32.1 | 0.084 |
| P16220 | 503.2726 | 2 | 5.5 | 32.5 | 0.06 |
| Q5TZA2 | 493.88 | 3 | −1.8 | 30.5 | 8.70E−04 |
| Q9BQ61 | 595.811 | 2 | 4.6 | 31.2 | 0.014 |
| Q9H6X5 | 782.024 | 3 | −14 | 32.4 | 2.60E−05 |
| Q13098 | 820.4062 | 3 | −2 | 61.5 | 1.10E−08 |
| Q9H175 | 672.7048 | 3 | −6.2 | 37.6 | 1.30E−05 |
| Q12996 | 652.331 | 4 | −6.2 | 39.9 | 2.20E−04 |
| Q8WYA6 | 781.3392 | 3 | −0.45 | 53.5 | 1.30E−07 |
| P49711 | 1069.8452 | 3 | −6.1 | 38.2 | 1.20E−07 |
| P49711 | 1069.8381 | 3 | −13 | 44.6 | 2.10E−07 |
| P49711 | 1064.5039 | 3 | −15 | 50.7 | 7.00E−09 |
| P35222 | 415.2119 | 3 | 2.6 | 29 | 0.0089 |
| O60716 | 747.7031 | 3 | −8.9 | 56.8 | 3.80E−06 |
| Q6PD62 | 498.719 | 4 | −7.4 | 40.8 | 5.00E−05 |
| Q13620 | 706.335 | 2 | −5.1 | 42.6 | 3.60E−05 |
| Q9NTM9 | 573.7935 | 2 | 4.2 | 40 | 8.60E−04 |
| P39880 | 962.4311 | 2 | 4.9 | 44.1 | 1.30E−05 |
| O43169 | 622.9469 | 3 | −32 | 33.8 | 7.10E−05 |
| P16989 | 510.6162 | 3 | 10 | 46.8 | 1.10E−06 |
| P16989 | 443.2429 | 2 | −0.47 | 27.6 | 0.0021 |
| P16989 | 892.1155 | 3 | 14 | 51.2 | 8.10E−06 |
| P16989 | 761.8609 | 2 | −8.2 | 27.4 | 0.0011 |
| P67809 | | | | | |
| Q9Y2T7 | | | | | |
| Q6PH85 | 533.2533 | 3 | 3.8 | 43.5 | 7.00E−05 |
| Q14203 | 607.9659 | 3 | 6.1 | 45.5 | 3.10E−05 |
| Q9NUU7 | 801.4011 | 2 | −14 | 26.5 | 0.014 |
| Q92499 | 705.915 | 2 | 0.84 | 31.6 | 2.50E−06 |
| Q9GZR7 | 1184.5992 | 2 | 1.4 | 34.7 | 2.00E−05 |
| Q7L014 | 809.1259 | 3 | 1.5 | 36.2 | 1.10E−04 |
| Q7L014 | 424.872 | 3 | 3.7 | 29.6 | 0.063 |
| Q7L014 | 755.8863 | 2 | −2 | 53.1 | 2.50E−05 |
| Q7L014 | 747.891 | 2 | 1 | 39.1 | 2.00E−04 |
| Q5T1V6 | 601.8233 | 2 | 1.4 | 40.5 | 0.0018 |
| P17661 | 490.2534 | 3 | −0.52 | 23.6 | 0.0019 |
| P17661 | 484.9203 | 3 | −3.5 | 29.6 | 0.011 |
| O00273 | 550.2886 | 2 | −3.9 | 28.9 | 0.022 |
| O00273 | 452.2561 | 2 | 17 | 36 | 0.037 |
| O00273 | 667.8635 | 4 | 2.2 | 22 | 0.041 |
| Q8WYQ5 | 643.3443 | 2 | −0.52 | 23.3 | 0.0055 |
| Q8WYQ5 | 926.1158 | 3 | −4.6 | 49.6 | 5.40E−09 |
| Q8WYQ5 | 920.7767 | 3 | −13 | 53 | 2.90E−09 |
| Q8WYQ5 | 987.1671 | 3 | 1.2 | 24 | 0.063 |
| Q86XP1 | 1012.8194 | 3 | −1.8 | 31.6 | 2.40E−04 |
| Q86XP1 | 702.3881 | 3 | −10 | 53.1 | 3.80E−09 |
| Q8NCG7 | 802.0843 | 3 | 1.3 | 63 | 1.10E−07 |
| Q3LXA3 | 543.7521 | 2 | −8.5 | 31.8 | 0.0026 |
| Q3LXA3 | 414.8688 | 3 | −12 | 35.1 | 3.30E−04 |
| Q7L2E3 | 519.2635 | 4 | 8.9 | 24.4 | 2.10E−04 |
| Q8IY37 | 996.1776 | 3 | −11 | 34 | 0.001 |
| Q08211 | 629.989 | 3 | 9.3 | 39.7 | 0.0011 |
| Q08211 | 890.1142 | 3 | −0.28 | 56.1 | 2.20E−09 |
| Q08211 | 884.7845 | 3 | 1.9 | 57.3 | 1.60E−09 |
| O60610 | 918.5204 | 4 | 11 | 35.9 | 9.30E−04 |
| Q9BTC0 | 865.1213 | 3 | −16 | 35.4 | 6.10E−05 |
| Q9BTC0 | 553.7901 | 2 | 16 | 33.1 | 8.20E−04 |
| Q9BTC0 | 796.7609 | 3 | 3.5 | 60.2 | 2.70E−08 |
| Q9BTC0 | 877.7758 | 3 | −26 | 45.1 | 5.70E−07 |
| Q9BTC0 | 399.519 | 3 | 4.9 | 24 | 0.012 |
| Q12959 | 1189.5762 | 3 | −0.17 | 25.8 | 0.0075 |
| Q99615 | 709.0066 | 3 | 2.2 | 34.6 | 5.40E−05 |
| Q99615 | 761.0354 | 3 | −4.4 | 40.5 | 1.00E−05 |
| Q99615 | 703.6769 | 3 | 5.1 | 66 | 1.10E−06 |
| Q99615 | 755.7119 | 3 | 6.4 | 55.9 | 1.10E−06 |
| O00429 | 665.3303 | 2 | −0.93 | 29.1 | 8.40E−05 |
| O00429 | 605.5967 | 3 | −2.2 | 51 | 1.50E−05 |
| O00429 | 657.6226 | 3 | −14 | 34.5 | 1.40E−04 |
| O00429 | 600.2747 | 3 | 14 | 49.7 | 1.40E−05 |
| O00429 | 652.2982 | 3 | −2.9 | 47.1 | 1.40E−04 |
| Q9Y6K1 | 573.5599 | 4 | 7.5 | 38.5 | 1.10E−04 |
| Q96BY6 | 709.3342 | 2 | −6.7 | 30.3 | 3.40E−06 |
| Q9BU89 | 563.8221 | 2 | −8.6 | 29 | 0.0021 |
| Q9BU89 | 607.3488 | 3 | −3.8 | 59.6 | 1.70E−07 |
| Q8TEK3 | 826.1048 | 3 | 2.2 | 30.7 | 0.0027 |
| Q9UKG1 | 1025.8137 | 3 | −23 | 53.4 | 1.20E−07 |
| P28340 | 599.9963 | 3 | 19 | 43.9 | 5.90E−07 |
| P28340 | 627.3408 | 4 | −11 | 36 | 1.80E−05 |
| P09884 | 525.7645 | 2 | 0.34 | 36.6 | 0.0066 |
| Q86TI2 | 604.7786 | 2 | −3.6 | 43.1 | 0.0049 |
| O14531 | 407.887 | 3 | 4.7 | 23.4 | 0.067 |
| Q16643 | 780.8959 | 2 | −1.2 | 23.7 | 0.0024 |
| Q16643 | 1009.9798 | 2 | −2.1 | 47.6 | 2.60E−05 |
| P55265 | 659.9648 | 3 | −5 | 39.8 | 4.40E−06 |
| Q9NZJ0 | 824.8967 | 4 | −7.2 | 41.2 | 3.10E−06 |
| Q9NZJ0 | 720.5584 | 5 | 4.3 | 44.5 | 1.80E−06 |
| Q8TDB6 | 660.31 | 2 | −1.6 | 24.5 | 0.0062 |
| Q14204 | 510.5649 | 3 | 0.026 | 28.9 | 0.0014 |
| Q14204 | 600.2835 | 2 | 1.4 | 35.5 | 0.072 |
| Q6ZTU2 | 936.4917 | 3 | −6.9 | 43 | 7.70E−05 |
| Q96L91 | | | | | |
| O43491 | 1026.8482 | 3 | −4.4 | 24.5 | 0.027 |
| Q9H1B7 | 812.954 | 2 | −0.63 | 34 | 1.70E−06 |
| Q99848 | 551.2859 | 3 | −0.34 | 44 | 4.20E−04 |
| P42892 | 813.3829 | 3 | −1.2 | 46.7 | 1.30E−04 |
| Q9H8V3 | 648.8259 | 2 | −2.4 | 22.2 | 0.013 |
| Q6P2E9 | 1036.5319 | 4 | 25 | 32.1 | 6.80E−04 |
| Q6P2E9 | 833.9083 | 2 | 1.7 | 36 | 1.10E−06 |
| Q6P2E9 | 825.9041 | 2 | −6.5 | 35.5 | 8.70E−07 |
| Q6P2E9 | 557.6229 | 3 | 0.72 | 27.7 | 0.036 |
| Q6P2E9 | 700.021 | 3 | −5.3 | 51.3 | 1.70E−07 |
| Q6P2E9 | 760.3679 | 3 | −3.6 | 55.8 | 6.80E−07 |
| Q6P2E9 | 428.8875 | 3 | −4.9 | 29.4 | 0.0077 |
| Q3B7T1 | 881.4229 | 2 | −6.5 | 39 | 1.50E−05 |
| Q3B7T1 | 945.4758 | 2 | −0.38 | 34.9 | 2.10E−07 |
| Q15075 | 907.7752 | 3 | −3.8 | 22.9 | 0.009 |
| Q15075 | 1124.5314 | 3 | −9.8 | 48.8 | 1.30E−07 |
| P68104 | 941.1216 | 3 | −7.7 | 44.6 | 1.40E−05 |
| Q5VTE0 | | | | | |
| P68104 | 790.0424 | 3 | −0.28 | 29.8 | 0.0025 |
| Q5VTE0 | | | | | |
| P68104 | 784.7067 | 3 | −5.5 | 43.6 | 2.90E−07 |
| Q5VTE0 | | | | | |
| P68104 | 674.3309 | 3 | 0.44 | 22.5 | 0.076 |
| Q5VTE0 | | | | | |
| P68104 | 479.2559 | 4 | −13 | 25.3 | 0.018 |
| Q5VTE0 | | | | | |
| P68104 | 850.3883 | 3 | −0.24 | 43.8 | 3.20E−06 |
| Q5VTE0 | | | | | |
| P24534 | 609.2763 | 3 | 3.1 | 45.2 | 4.80E−07 |
| P24534 | 917.8751 | 2 | −3.9 | 43.7 | 2.30E−05 |
| P29692 | 613.2911 | 4 | −0.12 | 33.8 | 1.70E−06 |
| P29692 | 734.3377 | 3 | −0.37 | 43.6 | 2.50E−05 |
| P13639 | 515.6041 | 3 | 4.1 | 63 | 3.40E−05 |
| Q8N3D4 | 951.4455 | 3 | 8.8 | 52.1 | 6.90E−08 |
| Q8NDI1 | 843.3957 | 3 | −2.4 | 45.2 | 1.10E−05 |
| Q9H4M9 | 1034.4543 | 3 | −3.1 | 52.4 | 3.10E−08 |
| Q9H9B1 | 576.9345 | 3 | −2.7 | 40.8 | 9.40E−05 |
| Q9H9B1 | 702.3415 | 2 | −0.92 | 28.2 | 1.10E−04 |
| Q96KQ7 | 491.9357 | 3 | 5.6 | 44 | 1.20E−04 |
| P55884 | 735.3623 | 2 | −3.9 | 42.1 | 0.0024 |
| P55884 | 683.7148 | 3 | −2 | 44 | 1.80E−04 |
| O75821 | 741.8482 | 4 | −2.3 | 38.9 | 1.10E−05 |
| O75822 | 667.7986 | 4 | −6.4 | 39.9 | 6.20E−06 |
| P32519 | 858.9174 | 2 | −12 | 60.5 | 2.40E−06 |
| P32519 | 850.9296 | 2 | −0.21 | 44.1 | 3.40E−06 |
| P06733 | 905.1272 | 3 | −14 | 34.6 | 4.50E−04 |
| P14625 | 554.7924 | 2 | 2.6 | 32.2 | 0.085 |
| P14625 | 599.2788 | 2 | −11 | 32.4 | 0.004 |
| P14625 | 480.912 | 3 | 17 | 30.3 | 1.90E−04 |
| Q9UBC2 | 806.7389 | 3 | 5.3 | 57.4 | 1.20E−06 |
| P42566 | 843.0613 | 3 | 9.5 | 29.7 | 0.0043 |
| Q9H2F5 | 594.3092 | 2 | −4.7 | 24 | 8.10E−05 |
| Q9Y6I3 | 538.6288 | 3 | 4.5 | 37.7 | 0.0043 |
| O95208 | 638.8329 | 2 | 4.2 | 32.2 | 5.10E−04 |
| Q2NKX8 | 551.6196 | 3 | −2.8 | 22.4 | 0.056 |
| Q03468 | 745.3792 | 2 | 5.7 | 23.2 | 0.0012 |
| P15170 | 674.8093 | 4 | −0.88 | 28.4 | 0.0016 |
| P15170 | 533.6556 | 5 | 7.9 | 35.3 | 1.30E−05 |
| P15170 | 569.8814 | 5 | 39 | 25 | 0.0013 |
| P15170 | 712.066 | 4 | −8.1 | 48.1 | 5.00E−06 |
| P50548 | 807.3873 | 2 | 1.2 | 27.3 | 0.0017 |
| Q86X53 | 531.277 | 2 | −0.54 | 26.6 | 0.0036 |
| A0FGR8 | 745.9169 | 2 | 3.5 | 43 | 0.022 |
| Q7Z2Z2 | 390.1911 | 3 | 15 | 31.6 | 3.60E−04 |
| Q7Z2Z2 | 589.2464 | 2 | 2.2 | 40.2 | 0.005 |
| Q9NVH0 | 440.2091 | 3 | 13 | 25.1 | 0.0051 |
| Q8N5W9 | 739.3938 | 3 | −8.5 | 42.5 | 1.00E−06 |
| Q9H098 | 660.663 | 3 | 1.7 | 31.1 | 7.20E−04 |
| Q6P1L5 | 438.2587 | 3 | 1.5 | 32.9 | 5.20E−04 |
| Q96EY5 | 466.9291 | 3 | 2 | 45.8 | 4.70E−04 |
| Q9Y6X4 | 667.8344 | 2 | −5.3 | 23.7 | 9.90E−04 |
| O94988 | 507.2676 | 3 | 3.8 | 31.8 | 0.0025 |
| O94988 | 567.6108 | 3 | −1.3 | 55.4 | 2.50E−06 |
| Q641Q2 | 809.892 | 2 | 3.7 | 49.8 | 2.60E−05 |
| Q5SNT6 | | | | | |
| Q9Y4E1 | | | | | |
| Q5SRD0 | | | | | |
| Q7Z4H7 | 671.3426 | 2 | −1.4 | 41.6 | 0.077 |
| Q8NFC6 | 840.4196 | 3 | −3 | 53.2 | 5.30E−08 |
| Q8NFC6 | 513.9373 | 3 | 6.4 | 40.2 | 3.50E−06 |
| Q8NFC6 | 758.3617 | 3 | −0.36 | 56.7 | 9.50E−08 |
| Q8NFC6 | 900.7601 | 3 | −8.8 | 65.1 | 3.60E−11 |
| Q8NFC6 | 753.034 | 3 | 4.8 | 67.6 | 1.20E−08 |
| Q8NFC6 | 581.7388 | 2 | 7.5 | 26.8 | 0.0022 |
| P49327 | 592.9841 | 3 | 1.5 | 46.1 | 2.00E−04 |
| P02765 | 480.8913 | 3 | −0.56 | 30.6 | 0.0083 |
| Q6UN15 | 438.8746 | 3 | −1.5 | 25.1 | 0.017 |
| Q5T1M5 | 1176.6186 | 3 | 0.9 | 32.4 | 1.60E−04 |
| Q5T1M5 | 994.8491 | 3 | 7.8 | 49 | 1.40E−06 |
| Q01543 | 445.8907 | 3 | −8.7 | 29.2 | 0.0027 |
| P21333 | 1143.0476 | 2 | 0.52 | 42.5 | 1.10E−08 |
| P21333 | 847.4108 | 3 | −15 | 30.1 | 6.30E−04 |
| P21333 | 768.7155 | 3 | −3.5 | 39.7 | 2.50E−05 |
| P21333 | 571.7673 | 2 | 21 | 33 | 0.0063 |
| P21333 | 736.361 | 2 | −1.9 | 41.3 | 4.70E−05 |
| P21333 | 775.7592 | 3 | 6.1 | 56.5 | 4.50E−08 |
| P21333 | 638.8862 | 4 | 43 | 50.2 | 7.30E−10 |
| P21333 | 793.4324 | 5 | 5.1 | 33.9 | 5.30E−06 |
| P21333 | 590.2847 | 2 | −5 | 38.7 | 0.048 |
| O75369 | | | | | |
| Q14315 | | | | | |
| O75369 | 484.2596 | 3 | 5 | 23.2 | 0.0062 |
| O75369 | 794.721 | 3 | −5.4 | 35.1 | 6.40E−04 |
| O75369 | 816.7023 | 3 | −19 | 62.4 | 1.10E−08 |
| O75369 | 811.3807 | 3 | −6.7 | 35.3 | 1.90E−05 |
| Q96RU3 | 949.8877 | 2 | 4.4 | 50.8 | 7.00E−09 |
| Q96RU3 | 941.8855 | 2 | −0.55 | 58.5 | 1.90E−07 |
| Q8N3X1 | 867.4791 | 3 | 1.3 | 39.8 | 1.60E−06 |
| Q8N3X1 | 459.7417 | 2 | 13 | 34.1 | 0.0046 |
| Q8N3X1 | 535.2756 | 2 | 6.3 | 35.1 | 5.30E−05 |
| Q9P0K8 | 479.7612 | 2 | 9 | 40.5 | 0.0011 |
| P85037 | 625.3471 | 3 | 7.3 | 60.1 | 9.10E−09 |
| O43524 | 1040.3843 | 2 | −15 | 43.7 | 5.70E−07 |
| Q8IVH2 | 636.952 | 5 | −2.6 | 42 | 1.70E−06 |
| P42345 | 429.552 | 3 | 3 | 42.5 | 0.0038 |
| P42345 | 472.2454 | 3 | −7.7 | 44.6 | 0.012 |
| O94915 | 508.5913 | 3 | −0.42 | 37 | 0.001 |
| Q96AE4 | 881.4463 | 2 | 0.8 | 48 | 9.20E−06 |
| Q96AE4 | 664.6509 | 3 | −0.6 | 35.5 | 0.001 |
| Q96AE4 | 483.2372 | 2 | −1.7 | 24 | 0.049 |
| Q92945 | | | | | |
| Q92945 | 579.3093 | 3 | 0.37 | 50.4 | 4.30E−06 |
| Q96I24 | 793.4292 | 2 | −18 | 42.7 | 3.90E−05 |
| Q96I24 | 912.4542 | 3 | −7.1 | 58.7 | 6.70E−07 |
| P35637 | 911.9938 | 2 | −0.33 | 39 | 5.90E−07 |
| P35637 | 442.8908 | 3 | −8.2 | 29.8 | 6.60E−04 |
| P51116 | 728.3358 | 4 | 10 | 32.3 | 7.10E−05 |
| O15117 | 1037.9759 | 2 | 2.9 | 26.4 | 2.70E−05 |
| O15117 | 912.7125 | 3 | −1.8 | 46 | 1.90E−07 |
| O15117 | 1007.7678 | 3 | 5.8 | 72.4 | 1.30E−09 |
| P06241 | 577.2888 | 2 | 9.2 | 41.4 | 1.50E−04 |
| Q96QD9 | 600.7898 | 2 | −7.8 | 41.3 | 0.016 |
| Q86V81 | | | | | |
| Q9Y2I7 | 663.8579 | 2 | 5.5 | 27.2 | 2.20E−05 |
| Q9Y2I7 | 792.423 | 2 | −0.051 | 41.6 | 9.10E−07 |
| Q9Y2I7 | 801.415 | 3 | −6.1 | 49.2 | 8.30E−07 |
| P04406 | 668.6631 | 3 | 5.8 | 33.8 | 4.90E−06 |
| Q06547 | 903.133 | 3 | −7.1 | 22.6 | 0.0016 |
| Q06547 | 955.1564 | 3 | −18 | 39.6 | 2.10E−04 |
| Q8TAK5 | 591.8463 | 2 | 1.1 | 33.2 | 5.50E−04 |
| P07902 | 479.244 | 2 | 2 | 29.4 | 0.047 |
| Q14C86 | 603.2872 | 3 | 6.2 | 52.3 | 6.50E−08 |
| Q14C86 | 531.257 | 4 | 5.4 | 37 | 5.80E−05 |
| P23769 | 667.8206 | 4 | 10 | 34.6 | 4.80E−05 |
| Q92538 | 788.3685 | 2 | 4.9 | 29.6 | 8.00E−06 |
| Q92538 | 586.2578 | 3 | −0.41 | 43.9 | 2.20E−07 |
| Q9Y5B6 | 369.5298 | 3 | 0.6 | 28.4 | 0.0087 |
| Q9BSJ2 | 857.7569 | 3 | −4.9 | 50.5 | 3.70E−04 |
| Q9H3P7 | 901.5111 | 4 | 48 | 33.2 | 2.80E−05 |
| Q9H3P7 | 803.0445 | 3 | 1.7 | 56 | 7.40E−08 |
| P52566 | 692.8988 | 2 | −3.2 | 23.3 | 4.80E−04 |
| P52566 | 429.0122 | 4 | 12 | 31.5 | 1.90E−04 |
| P52566 | 531.7784 | 2 | 2.7 | 35.2 | 0.014 |
| P06396 | 607.7944 | 2 | −2.1 | 41.5 | 0.0065 |
| P06396 | 656.0097 | 3 | 6 | 55.1 | 1.10E−05 |
| Q8TEQ6 | 962.1116 | 3 | −8.3 | 38.8 | 7.50E−05 |
| Q9NWZ8 | 659.951 | 3 | −0.54 | 42 | 1.70E−06 |
| Q17RS7 | 940.4229 | 2 | −5.6 | 23.4 | 0.0081 |
| Q06210 | 698.3406 | 2 | −6.1 | 37.1 | 3.20E−05 |
| Q9NZ52 | 1129.5866 | 3 | −26 | 37.9 | 1.10E−04 |
| Q9NZ52 | 690.3595 | 2 | 4.6 | 48.7 | 0.0065 |
| Q9Y2X7 | 814.0902 | 3 | −11 | 29.1 | 0.0062 |
| Q9Y2X7 | 807.43 | 2 | 6.6 | 49.5 | 4.00E−04 |
| Q9Y2X7 | 581.3222 | 3 | 8.6 | 42 | 8.70E−05 |
| Q9Y2X7 | 959.9743 | 2 | −12 | 39.5 | 1.60E−04 |
| Q14161 | 789.7401 | 3 | 9.8 | 53.7 | 1.20E−04 |
| Q04446 | 454.1991 | 3 | 6.4 | 30 | 6.20E−04 |
| O76003 | 391.8701 | 3 | 5 | 31.2 | 0.0041 |
| O76003 | 434.562 | 3 | −10 | 33.6 | 0.0058 |
| P14314 | 966.4078 | 2 | −1.3 | 33.4 | 1.20E−06 |
| P14314 | 730.3231 | 3 | 2.8 | 56.6 | 7.40E−08 |
| P14314 | 1200.0392 | 4 | −16 | 25.1 | 3.00E−05 |
| P14314 | 1019.0646 | 3 | −1.6 | 38.7 | 3.90E−07 |
| Q9P107 | 1023.4906 | 2 | 0.26 | 35.9 | 8.30E−07 |
| Q9P107 | 460.7289 | 2 | 8 | 38.5 | 0.0034 |
| Q9P107 | 713.3512 | 2 | −4 | 35.2 | 9.90E−05 |
| P36915 | 694.827 | 2 | −0.48 | 36.4 | 6.90E−05 |
| P36915 | 820.6503 | 4 | −10 | 32.4 | 5.60E−04 |
| P36915 | 429.2062 | 3 | −19 | 32.8 | 0.03 |
| P36915 | 785.3473 | 2 | 1.5 | 37.5 | 1.10E−05 |
| P36915 | 530.2459 | 3 | −7.5 | 31.4 | 0.0016 |
| Q14789 | 907.4273 | 3 | −15 | 46.6 | 1.20E−06 |
| Q14789 | 670.6572 | 3 | −3 | 29.8 | 0.0048 |
| Q14789 | 730.9991 | 3 | −8.1 | 49.8 | 2.80E−06 |
| Q3T8J9 | 806.3869 | 2 | −11 | 33.4 | 7.90E−04 |
| Q92917 | 487.2541 | 3 | 3.5 | 40.5 | 1.70E−05 |
| Q92917 | 561.2759 | 2 | 30 | 31.5 | 0.018 |
| Q92917 | 542.5193 | 4 | −5.1 | 40.6 | 1.00E−07 |
| Q92917 | 519.2812 | 2 | −7.2 | 28.6 | 0.0096 |
| Q92917 | 603.3236 | 3 | −13 | 43 | 1.10E−04 |
| Q9HCN4 | 578.3334 | 3 | −5.7 | 33.2 | 1.40E−04 |
| Q9UKJ3 | 892.4116 | 3 | −21 | 43.8 | 8.80E−06 |
| Q3V6T2 | 626.54 | 4 | −4.4 | 51.6 | 4.20E−06 |
| Q3V6T2 | 521.2493 | 2 | 6.4 | 34.8 | 0.061 |
| Q7Z2K8 | 715.8928 | 2 | −4.6 | 23 | 0.0017 |
| P57764 | 819.9452 | 2 | 1.3 | 36.9 | 4.10E−06 |
| P57764 | 889.9305 | 2 | −9.6 | 40 | 4.90E−06 |
| P09211 | 624.7982 | 2 | 0.18 | 30.9 | 0.0026 |
| P09211 | 616.7935 | 2 | −12 | 41 | 5.70E−04 |
| P78347 | 590.8117 | 2 | −4 | 41.8 | 0.042 |
| P78347 | 436.9094 | 3 | −1.5 | 38 | 0.044 |
| O75367 | 898.9377 | 2 | −13 | 44.9 | 2.30E−05 |
| P62805 | 552.7883 | 2 | 0.87 | 22.5 | 0.0064 |
| P62805 | 420.9007 | 3 | 14 | 36.2 | 8.00E−04 |
| P62805 | 463.5985 | 3 | 12 | 37.5 | 4.60E−04 |
| P62805 | 432.605 | 3 | 16 | 39.8 | 2.10E−04 |
| Q13442 | 376.8892 | 3 | 14 | 23.9 | 0.017 |
| Q13442 | 452.5993 | 3 | 5.9 | 28.8 | 0.0026 |
| Q9Y450 | 1007.4704 | 2 | −2 | 40.9 | 7.90E−05 |
| P14317 | 483.9191 | 3 | 6.6 | 46.6 | 3.10E−04 |
| P14317 | 601.2884 | 2 | −3.3 | 35 | 0.048 |
| P56524 | 603.0072 | 3 | 14 | 38 | 9.30E−05 |
| P56524 | 1383.3258 | 3 | 1.1 | 33.9 | 6.30E−05 |
| Q9UBN7 | 596.2706 | 2 | 17 | 34.3 | 0.0016 |
| Q9UBN7 | 580.2673 | 2 | 3.3 | 39 | 0.014 |
| Q8WUI4 | 581.6054 | 3 | −4.4 | 45.4 | 3.20E−06 |
| Q9UBI9 | 440.8914 | 3 | −7.4 | 35.5 | 0.0021 |
| Q7Z4V5 | 737.1483 | 4 | 0.079 | 22.1 | 0.035 |
| Q7Z4V5 | 464.9002 | 3 | −5.8 | 38.6 | 5.40E−05 |
| Q7Z4V5 | 459.5666 | 3 | −10 | 23.2 | 0.012 |
| Q7Z4V5 | 386.5469 | 3 | −4.4 | 31.6 | 0.0041 |
| Q9ULT8 | 897.4596 | 3 | −2.3 | 28.6 | 0.0036 |
| Q9NRZ9 | 829.0873 | 3 | −0.68 | 35.6 | 3.60E−04 |
| P04233 | 875.9403 | 2 | −3.4 | 48 | 9.20E−05 |
| P04233 | 867.9434 | 2 | −2.7 | 47.9 | 3.30E−04 |
| Q9BW71 | 515.2498 | 2 | −11 | 24.4 | 0.0058 |
| Q8NCD3 | 583.3096 | 3 | 15 | 44 | 9.40E−07 |
| Q92619 | 920.6968 | 4 | −14 | 26.5 | 1.20E−04 |
| Q92619 | 960.4808 | 2 | 0.58 | 43.9 | 1.20E−05 |
| Q92619 | 973.9817 | 2 | −15 | 38.1 | 1.10E−05 |
| P30519 | 369.5154 | 3 | 4.6 | 29.4 | 0.001 |
| P30519 | 527.8961 | 3 | −24 | 35.5 | 3.60E−06 |
| P30519 | 392.1869 | 4 | 6 | 22.9 | 0.0017 |
| P31943 | 871.9425 | 2 | −0.65 | 34.4 | 9.70E−05 |
| P31943 | 513.8811 | 5 | 17 | 32.1 | 2.10E−04 |
| P55795 | | | | | |
| P31943 | 766.0473 | 3 | −8.5 | 44.4 | 7.00E−06 |
| P55795 | | | | | |
| P31943 | 554.7388 | 2 | 0.87 | 33.5 | 2.90E−04 |
| P55795 | | | | | |
| P55795 | 581.6278 | 3 | −5.7 | 24.8 | 0.037 |
| P31942 | 934.0253 | 3 | −7.3 | 40.1 | 3.20E−08 |
| Q9BUJ2 | 481.8748 | 3 | −15 | 42.2 | 1.70E−05 |
| Q9BUJ2 | 476.5497 | 3 | −1.9 | 45.5 | 6.20E−05 |
| Q1KMD3 | 851.0696 | 3 | 1.4 | 62.6 | 7.50E−09 |
| Q1KMD3 | 677.5804 | 4 | 3 | 53.3 | 7.20E−07 |
| Q8WVV9 | 567.6299 | 3 | −0.24 | 37.5 | 0.0035 |
| Q14103 | 499.2117 | 4 | −12 | 41 | 7.40E−07 |
| P52597 | 742.3466 | 2 | 0.49 | 43.3 | 4.60E−04 |
| P38159 | 574.2536 | 2 | −1.3 | 36.1 | 1.50E−05 |
| P38159 | 495.25 | 3 | 1.8 | 31.5 | 0.0024 |
| P61978 | 755.3641 | 2 | 3.7 | 30.5 | 2.80E−05 |
| P61978 | 648.0446 | 4 | 2.7 | 55.5 | 2.80E−10 |
| P61978 | 1167.8252 | 3 | −10 | 22.2 | 7.80E−05 |
| P61978 | 512.2341 | 2 | 7.6 | 26.4 | 0.0056 |
| P14866 | 756.0158 | 3 | −6.8 | 34.1 | 1.90E−04 |
| O60506 | 555.2274 | 3 | −8.2 | 37 | 8.20E−05 |
| Q9UJC3 | 750.3508 | 2 | −4.7 | 47.6 | 5.80E−04 |
| Q9UJC3 | 543.2718 | 3 | 2.9 | 35.7 | 7.70E−04 |
| Q96ED9 | 886.8975 | 2 | −12 | 48.5 | 2.10E−04 |
| Q9NQG7 | 1106.7951 | 3 | −14 | 55.7 | 8.90E−10 |
| Q03164 | 898.9001 | 2 | −12 | 52.9 | 4.30E−07 |
| Q03164 | 642.3179 | 3 | 15 | 53.2 | 1.90E−06 |
| Q03164 | 972.9138 | 2 | −5.2 | 46.5 | 2.70E−06 |
| Q92598 | 770.0305 | 3 | 8.3 | 53.5 | 1.90E−07 |
| Q92598 | 983.132 | 3 | 0.38 | 44 | 3.50E−06 |
| Q92598 | 848.1935 | 4 | 45 | 49 | 2.60E−07 |
| P34931 | 449.2161 | 3 | 15 | 31.2 | 0.0019 |
| P08107 | | | | | |
| P54652 | | | | | |
| P17066 | | | | | |
| P48741 | | | | | |
| P11142 | | | | | |
| P34932 | 451.7001 | 2 | 0.47 | 25.9 | 0.013 |
| P11142 | 481.7543 | 2 | 3.9 | 29.1 | 0.011 |
| Q99081 | 727.3534 | 2 | −10 | 28.4 | 0.0028 |
| O43719 | 911.4252 | 3 | −2.8 | 42 | 4.90E−06 |
| O43719 | 1121.2057 | 3 | 0.38 | 23.7 | 0.092 |
| O43719 | 529.2607 | 3 | 6.9 | 39.2 | 3.90E−07 |
| O43719 | 736.0272 | 3 | 0.37 | 55.5 | 1.70E−08 |
| Q7Z6Z7 | 616.8288 | 2 | −7.3 | 26.6 | 0.0025 |
| Q7Z6Z7 | 656.8401 | 2 | −1.1 | 24.5 | 1.50E−04 |
| Q7Z6Z7 | 486.7675 | 2 | −1.8 | 31.8 | 7.00E−04 |
| Q7Z6Z7 | 559.7913 | 2 | −4.1 | 31.5 | 1.40E−04 |
| Q7Z6Z7 | 826.3566 | 3 | 0.28 | 57.6 | 1.20E−08 |
| Q7Z6Z7 | 701.367 | 3 | −4.1 | 59.6 | 1.10E−07 |
| Q7Z5L9 | 683.3163 | 3 | −0.65 | 57 | 9.60E−08 |
| P32019 | 496.2696 | 2 | 0.067 | 31.9 | 0.07 |
| Q8WUF5 | 581.6511 | 3 | −0.51 | 44.7 | 3.60E−06 |
| P20810 | 827.8799 | 2 | −13 | 56.2 | 1.50E−07 |
| P20810 | 594.9629 | 3 | 2.8 | 54.1 | 4.20E−08 |
| P20810 | 950.9444 | 2 | −5.7 | 50.5 | 1.40E−05 |
| P20810 | 492.0218 | 4 | −7.7 | 39 | 6.40E−07 |
| P20810 | 881.3723 | 2 | −13 | 52.4 | 1.20E−06 |
| A6NK07 | 471.2361 | 2 | −12 | 28.9 | 0.063 |
| P20042 | | | | | |
| O60841 | 628.8424 | 2 | −5.6 | 48.6 | 6.20E−05 |
| O60841 | 666.0137 | 3 | −3.9 | 58.6 | 7.80E−06 |
| O60841 | 553.5485 | 4 | 4.7 | 48.4 | 1.60E−06 |
| Q14240 | 873.395 | 3 | −0.38 | 56.2 | 2.40E−06 |
| Q14240 | 1067.4832 | 3 | −27 | 48 | 8.40E−08 |
| P23588 | 522.9046 | 3 | −35 | 23.3 | 0.051 |
| P23588 | 648.9545 | 3 | 6.7 | 41.1 | 1.00E−04 |
| Q04637 | 1290.1025 | 4 | 0.97 | 22.1 | 0.028 |
| Q04637 | 680.3351 | 2 | −4.5 | 43.7 | 9.10E−04 |
| Q04637 | 782.3865 | 2 | 7.7 | 49.6 | 0.01 |
| P78344 | 544.9423 | 3 | 3 | 49 | 8.90E−06 |
| P78344 | 1015.8857 | 3 | 4.5 | 27.8 | 0.0033 |
| O43432 | 596.9612 | 3 | −1.8 | 44.8 | 3.30E−06 |
| O43432 | 648.9964 | 3 | 0.65 | 38.5 | 0.0019 |
| Q15056 | 903.9804 | 2 | −3.3 | 40.4 | 4.10E−06 |
| P63241 | 761.391 | 2 | −10 | 39 | 3.30E−04 |
| P63241 | 834.8914 | 2 | −4.6 | 30.1 | 1.10E−05 |
| Q6IS14 | | | | | |
| P63241 | 594.304 | 3 | 7.9 | 47.1 | 1.80E−05 |
| Q6IS14 | | | | | |
| P63241 | 826.8934 | 2 | −5.3 | 42.9 | 1.90E−05 |
| Q6IS14 | | | | | |
| P63241 | 782.6949 | 3 | −6.5 | 54.8 | 2.50E−10 |
| Q6IS14 | | | | | |
| P63241 | 654.6412 | 3 | −6 | 35 | 3.20E−04 |
| Q6IS14 | | | | | |
| P63241 | 795.0175 | 3 | 2.1 | 57.1 | 1.90E−06 |
| Q6IS14 | | | | | |
| P63241 | 832.3709 | 3 | −14 | 60.6 | 2.50E−09 |
| Q6IS14 | | | | | |
| P63241 | 789.6877 | 3 | 4.4 | 59.2 | 4.20E−07 |
| Q6IS14 | | | | | |
| Q9GZV4 | 778.6994 | 3 | −0.68 | 54.2 | 1.50E−07 |
| Q9GZV4 | 839.0477 | 3 | 2.3 | 35.9 | 4.20E−05 |
| Q15653 | 635.8092 | 2 | 3.8 | 43.4 | 0.0031 |
| Q96HA7 | 702.685 | 3 | −2.8 | 47.6 | 7.60E−08 |
| Q96HA7 | 566.2926 | 4 | 0.63 | 41.3 | 9.40E−06 |
| Q13422 | 718.8969 | 2 | −9.1 | 57.1 | 0.002 |
| Q9UKS7 | 819.8802 | 2 | 6.3 | 38 | 1.50E−05 |
| Q9H5V7 | 541.2358 | 2 | 13 | 27.3 | 0.0031 |
| Q12906 | 598.0347 | 4 | 3.4 | 39.8 | 2.50E−08 |
| Q12906 | 665.7981 | 2 | −1.5 | 47.2 | 5.40E−05 |
| Q12906 | 486.9006 | 3 | 0.88 | 30.9 | 7.80E−05 |
| Q9H0C8 | 1055.1788 | 3 | −7.7 | 33.7 | 2.00E−05 |
| P52294 | 671.91 | 5 | −7.6 | 23.1 | 0.011 |
| P52294 | 1168.8793 | 3 | 8.3 | 56.1 | 1.40E−07 |
| O60684 | 868.9194 | 2 | −1.2 | 48.8 | 1.80E−05 |
| P12268 | 628.3016 | 2 | −8.2 | 27.5 | 0.0019 |
| Q53TQ3 | 592.8169 | 2 | −0.42 | 31.4 | 0.0017 |
| Q27J81 | 852.7746 | 3 | −13 | 28.6 | 4.10E−04 |
| Q27J81 | 678.866 | 4 | −0.28 | 32.1 | 9.10E−05 |
| Q27J81 | 491.5756 | 3 | 10 | 32.8 | 6.10E−06 |
| Q96P70 | 868.9808 | 2 | 6.6 | 46.8 | 1.90E−04 |
| Q6DN90 | 668.841 | 2 | 0.35 | 37.6 | 3.80E−06 |
| P46940 | 523.2617 | 4 | −7.9 | 43 | 1.20E−06 |
| P46940 | 791.8718 | 4 | −13 | 48.2 | 9.70E−10 |
| P14316 | 481.7287 | 4 | −0.69 | 37.4 | 6.40E−07 |
| O14654 | 569.2558 | 3 | −1.8 | 56.2 | 4.30E−05 |
| Q9ULR0 | 744.8934 | 2 | −10 | 35 | 0.002 |
| Q9ULR0 | 594.651 | 3 | 3.6 | 35.8 | 0.0022 |
| Q96ST2 | 495.5329 | 3 | −1.1 | 40.8 | 3.40E−06 |
| Q96ST2 | 367.9039 | 4 | 1.8 | 27.4 | 0.0015 |
| Q9H3R0 | 821.4007 | 2 | −1.5 | 37.8 | 1.40E−06 |
| Q9H3R0 | 695.6855 | 3 | 3.5 | 47 | 1.90E−06 |
| O60271 | 596.8115 | 2 | −4.4 | 42.8 | 0.047 |
| O60271 | 706.6559 | 3 | 4.1 | 46.4 | 3.10E−08 |
| O60271 | 701.3263 | 3 | 7 | 64.2 | 2.30E−08 |
| O60271 | 780.0608 | 3 | −5.7 | 35.4 | 0.035 |
| Q96N16 | 623.3181 | 2 | 2.7 | 36.2 | 0.0012 |
| Q96N16 | 713.8268 | 2 | −12 | 34.1 | 1.30E−04 |
| Q8N9B5 | 657.3692 | 2 | −0.032 | 35.9 | 1.10E−04 |
| Q8N9B5 | 724.3729 | 3 | −6.9 | 55.5 | 8.50E−09 |
| Q9H5J8 | 466.25 | 3 | 13 | 31.1 | 0.0022 |
| Q96MG2 | 865.3917 | 3 | 7 | 52.6 | 1.80E−05 |
| P53990 | 565.7727 | 2 | 1.5 | 25.2 | 0.04 |
| P53990 | 420.2057 | 3 | −23 | 29.6 | 0.097 |
| Q92628 | 502.7451 | 2 | −0.04 | 23.9 | 0.035 |
| Q5JSZ5 | 825.9764 | 2 | −0.66 | 25.7 | 3.50E−04 |
| Q5JSZ5 | 416.8733 | 3 | −5.2 | 29.4 | 0.0034 |
| Q6ZNE5 | 804.3995 | 2 | 0.31 | 29.9 | 7.30E−05 |
| Q6ZNE5 | 632.7896 | 2 | −0.53 | 57.2 | 8.80E−04 |
| Q9P266 | 772.3801 | 3 | −1.3 | 43.4 | 1.10E−05 |
| Q9P1Y5 | 1094.9782 | 2 | 0.65 | 37.3 | 1.80E−07 |
| Q9HCE5 | 501.2751 | 3 | 9.2 | 57.5 | 1.90E−04 |
| Q9HCE5 | 551.0467 | 4 | 6 | 50.3 | 2.00E−07 |
| Q9HCE5 | 569.8155 | 2 | 12 | 34.1 | 0.009 |
| Q8IXQ4 | 742.8841 | 2 | 2.5 | 49.2 | 1.00E−04 |
| Q8N163 | 965.44 | 2 | 0.78 | 51.8 | 9.40E−06 |
| Q8N163 | 771.413 | 3 | 3 | 42.2 | 3.30E−04 |
| Q8N163 | 544.0272 | 4 | −12 | 24.2 | 0.0032 |
| Q07666 | 1009.0557 | 2 | −14 | 51 | 2.30E−08 |
| P46013 | 456.7362 | 4 | −3.1 | 45.5 | 3.00E−06 |
| P46013 | 471.2396 | 3 | −1.6 | 39.3 | 4.40E−04 |
| P46013 | 497.7349 | 4 | −2 | 33.8 | 5.00E−04 |
| Q9NS87 | 640.3066 | 2 | 20 | 36.9 | 0.0064 |
| Q8N5S9 | 482.235 | 2 | 10 | 25.9 | 0.037 |
| Q9Y4X4 | 669.6589 | 3 | −9.8 | 48.2 | 8.50E−05 |
| P14618 | 813.389 | 3 | −6.8 | 33.9 | 5.90E−04 |
| P14618 | 719.3419 | 2 | 3.7 | 24.7 | 5.20E−04 |
| P30613 | | | | | |
| Q8N9T8 | 365.2104 | 3 | 15 | 22.8 | 0.0065 |
| Q13601 | 433.882 | 3 | −4.2 | 36.2 | 0.0065 |
| Q13601 | 478.4787 | 4 | −5.2 | 41.7 | 3.70E−06 |
| O75676 | 446.5743 | 3 | 12 | 37.8 | 4.00E−04 |
| P13010 | 567.3097 | 2 | −10 | 36.9 | 0.0011 |
| P13010 | 657.839 | 2 | 7.5 | 33.5 | 2.40E−05 |
| P13010 | 406.8455 | 3 | −14 | 31.1 | 1.00E−03 |
| Q14657 | 625.3109 | 3 | −6.5 | 53.5 | 5.80E−07 |
| P07942 | 603.2805 | 3 | −4.7 | 27.7 | 0.074 |
| P42166 | 563.9676 | 3 | 2.1 | 27.5 | 0.0043 |
| P42166 | 763.7119 | 3 | −0.39 | 28.9 | 7.80E−04 |
| Q14160 | 711.3793 | 3 | −18 | 43.8 | 5.20E−06 |
| Q14160 | 462.2459 | 3 | 12 | 34.1 | 0.022 |
| Q14160 | 600.0339 | 4 | −5.6 | 44.1 | 7.30E−09 |
| Q6PKG0 | 561.9504 | 3 | −7.3 | 49.7 | 3.50E−04 |
| Q6PKG0 | 619.6416 | 3 | 5.6 | 53.1 | 3.60E−05 |
| Q71RC2 | 744.3937 | 3 | −0.062 | 49.5 | 1.40E−06 |
| Q92615 | 1019.1531 | 3 | −0.13 | 24.9 | 0.029 |
| O43561 | 1020.0438 | 2 | 1.7 | 22.7 | 4.00E−04 |
| Q9UIQ6 | 627.3172 | 4 | 7.4 | 40.6 | 6.90E−06 |
| Q8N3X6 | 541.7993 | 2 | 17 | 27 | 0.011 |
| Q96JN0 | | | | | |
| Q9UHB6 | 810.1002 | 3 | −3.4 | 40.6 | 3.00E−06 |
| Q96GY3 | 662.349 | 2 | −5.2 | 40.5 | 0.05 |
| Q9NUP9 | 562.7991 | 2 | 29 | 28.8 | 0.066 |
| Q13136 | 1005.9911 | 2 | 1.1 | 38.3 | 2.30E−07 |
| Q13136 | 1084.037 | 2 | −3.2 | 39.1 | 1.10E−06 |
| Q8ND30 | 1023.8431 | 3 | −0.77 | 26.9 | 0.0098 |
| P20700 | 572.3392 | 2 | −1.8 | 26 | 3.60E−05 |
| Q8WWI1 | 728.8589 | 2 | −12 | 52.3 | 0.0013 |
| Q8IWU2 | 622.8452 | 2 | −1.1 | 39.6 | 3.30E−04 |
| Q9C0E8 | 617.0009 | 3 | 9.8 | 35.9 | 4.70E−04 |
| Q93052 | 763.8397 | 2 | 9.2 | 22.5 | 1.40E−04 |
| Q93052 | 755.8336 | 2 | −2.1 | 27.4 | 8.00E−05 |
| P50851 | 761.3326 | 2 | −1.3 | 31.9 | 9.80E−07 |
| P50851 | 851.8504 | 2 | −2.2 | 51.5 | 9.90E−09 |
| P50851 | 639.3044 | 3 | 15 | 61 | 4.10E−05 |
| P50851 | 633.9662 | 3 | 4.5 | 49.5 | 9.80E−05 |
| Q8N1G4 | 945.4597 | 2 | −6.2 | 46.9 | 2.80E−06 |
| Q8N1G4 | 796.4119 | 4 | −2.2 | 59 | 2.90E−10 |
| Q9Y2L9 | 655.8126 | 2 | −1.4 | 26.2 | 8.30E−05 |
| Q9Y2L9 | 513.2605 | 3 | 5.9 | 29.5 | 6.80E−04 |
| Q5VUJ6 | 395.7015 | 4 | −11 | 39.7 | 7.80E−05 |
| Q96II8 | 474.2556 | 2 | 14 | 36.1 | 0.013 |
| O75427 | 413.8601 | 3 | 4.7 | 27.9 | 0.0014 |
| Q12912 | 738.9125 | 2 | −8.9 | 48.2 | 0.0057 |
| Q32MZ4 | 1070.7324 | 4 | 0.24 | 32.4 | 4.30E−04 |
| Q9Y608 | 1281.3087 | 3 | −2.3 | 30.6 | 0.0028 |
| P83369 | 520.261 | 2 | −1.8 | 22.6 | 7.10E−04 |
| P62310 | 985.5148 | 2 | 1.7 | 26.1 | 7.90E−04 |
| P33241 | 590.7599 | 2 | 6.2 | 35.6 | 0.025 |
| Q96GA3 | 537.006 | 4 | −2 | 22.3 | 0.035 |
| Q86UE4 | 717.348 | 2 | −1.4 | 38 | 9.00E−06 |
| O60664 | 742.8439 | 2 | −22 | 44.9 | 9.60E−06 |
| O60664 | 769.0493 | 3 | 1.2 | 60 | 1.60E−08 |
| O60664 | 583.3055 | 2 | 6.7 | 47.7 | 0.063 |
| Q3KQU3 | 1073.2181 | 3 | −3.9 | 30.4 | 0.0011 |
| Q3KQU3 | 1067.8871 | 3 | −3.3 | 28.9 | 0.005 |
| Q9UPN3 | 829.739 | 3 | −8 | 46.7 | 1.70E−06 |
| Q9UPN3 | 699.8616 | 4 | −1 | 30.7 | 7.80E−04 |
| Q96PK2 | | | | | |
| Q9UPN3 | 838.0933 | 3 | −1 | 33 | 8.60E−05 |
| Q96PK2 | | | | | |
| Q9UPN3 | 611.2847 | 4 | −4.7 | 50.5 | 1.10E−08 |
| Q96PK2 | | | | | |
| Q9UPN3 | 817.9101 | 2 | −4.8 | 34.2 | 2.90E−05 |
| Q96PK2 | | | | | |
| Q9UPN3 | 639.8107 | 2 | −0.49 | 37.5 | 1.90E−06 |
| Q96PK2 | | | | | |
| Q9UPN3 | 605.9573 | 3 | −0.38 | 49.3 | 1.50E−06 |
| Q96PK2 | | | | | |
| Q9UPN3 | 730.334 | 2 | 5.9 | 41 | 6.30E−04 |
| Q96PK2 | | | | | |
| Q8WXG6 | 657.3757 | 2 | −13 | 34.7 | 0.0096 |
| Q9Y5V3 | 714.665 | 3 | 5.1 | 59.6 | 1.10E−06 |
| Q96MG7 | 701.316 | 2 | −2.3 | 48.5 | 2.70E−04 |
| Q96MG7 | 969.7688 | 3 | −12 | 52.3 | 1.20E−07 |
| P23368 | 608.0444 | 3 | 15 | 33.6 | 2.00E−04 |
| P78559 | 1157.6109 | 2 | 9.5 | 38.8 | 1.40E−06 |
| P78559 | 916.7866 | 3 | −14 | 53.3 | 1.60E−06 |
| P78559 | 719.8776 | 4 | 3.4 | 52.3 | 6.50E−08 |
| P27816 | 792.4116 | 2 | −0.25 | 28.1 | 4.50E−04 |
| P27816 | 580.6472 | 3 | 5.6 | 51.1 | 7.80E−06 |
| P27816 | 497.9203 | 3 | −5.6 | 25.8 | 0.028 |
| P27816 | 843.7541 | 3 | −1.2 | 47.2 | 4.40E−05 |
| P27816 | 730.7259 | 3 | −0.99 | 45 | 8.80E−05 |
| P27816 | 727.8715 | 2 | −3.9 | 42 | 0.0061 |
| Q49MG5 | 502.7553 | 2 | −4.7 | 30.1 | 0.041 |
| Q15691 | 547.2662 | 3 | 7.8 | 26.6 | 0.0011 |
| Q9P0L2 | 507.587 | 3 | 5.5 | 38.4 | 1.60E−04 |
| P43243 | 1065.4672 | 2 | 0.62 | 38.4 | 7.70E−09 |
| P43243 | 622.8264 | 2 | −2.2 | 36.2 | 1.30E−06 |
| P43243 | 686.8753 | 2 | 0.15 | 36.4 | 3.30E−06 |
| P43243 | 583.7786 | 4 | −1.2 | 39.6 | 2.10E−05 |
| P43243 | 600.0053 | 3 | 6.5 | 41.5 | 1.20E−05 |
| P43243 | 456.7237 | 2 | −2.3 | 25.6 | 0.018 |
| P43243 | 524.2863 | 4 | 42 | 54.5 | 3.80E−07 |
| Q7Z434 | 401.8584 | 3 | −22 | 30.1 | 0.0099 |
| Q7Z434 | 333.6772 | 4 | 4.1 | 28.2 | 0.031 |
| P61244 | 515.2815 | 2 | −1.7 | 27.2 | 0.072 |
| Q9BQG0 | 726.3666 | 3 | −7.6 | 44.5 | 6.80E−06 |
| P49736 | 837.0505 | 3 | −8.2 | 35.4 | 2.70E−05 |
| P49736 | 645.802 | 2 | −2.1 | 28.9 | 0.014 |
| P25205 | 927.726 | 3 | 4.5 | 45.9 | 9.50E−08 |
| P33991 | 586.9691 | 3 | 3.3 | 48 | 3.90E−07 |
| P33992 | 541.2561 | 3 | −1.6 | 53 | 6.00E−07 |
| P33992 | 829.8377 | 2 | −22 | 48.5 | 2.30E−07 |
| Q14566 | 445.9049 | 3 | −0.98 | 38.8 | 0.0012 |
| Q14566 | 587.765 | 2 | −13 | 32 | 0.0024 |
| Q14676 | 1192.6804 | 4 | 5.2 | 25.9 | 3.50E−04 |
| Q14676 | 935.8055 | 3 | −7.9 | 30.8 | 0.0041 |
| Q9NU22 | 939.4534 | 3 | 7.9 | 45.5 | 7.80E−07 |
| O60244 | 728.3893 | 3 | 1.7 | 49.9 | 1.40E−04 |
| Q15648 | 586.3193 | 2 | −3.7 | 39 | 0.038 |
| Q15648 | 547.283 | 3 | 1.2 | 36.1 | 4.80E−04 |
| O95402 | 487.5985 | 3 | −3.2 | 51.1 | 3.20E−04 |
| Q06413 | 474.5466 | 3 | 5.2 | 32.5 | 8.80E−04 |
| Q06413 | 435.9476 | 4 | −7.9 | 33.2 | 3.70E−06 |
| P31153 | 803.4165 | 2 | 1.7 | 25.7 | 0.001 |
| Q6ZN04 | 1048.8488 | 3 | −9.3 | 26.4 | 3.70E−04 |
| Q8IWI9 | 1088.4852 | 2 | −0.27 | 31.4 | 1.00E−06 |
| Q8IWI9 | 578.8284 | 2 | −6.6 | 36.8 | 0.018 |
| Q8IWI9 | 1041.8352 | 3 | −7.7 | 33 | 2.50E−04 |
| Q8IWI9 | 502.26 | 3 | 3.5 | 57 | 2.80E−05 |
| Q5JRA6 | 525.2844 | 2 | −1.1 | 33.7 | 3.90E−04 |
| Q8N108 | 454.5474 | 3 | 9.1 | 31.6 | 9.90E−05 |
| Q96T58 | 499.6148 | 3 | 5.4 | 40.8 | 9.70E−05 |
| Q96T58 | 668.6869 | 3 | 3.2 | 51.5 | 3.20E−05 |
| Q96T58 | 626.3152 | 2 | 1.3 | 44 | 8.50E−04 |
| Q8NDC0 | 400.5423 | 3 | 5.7 | 27.1 | 0.0033 |
| Q969V6 | 1230.1066 | 2 | −4.2 | 24.9 | 1.30E−05 |
| Q9ULH7 | 840.3932 | 3 | 4.7 | 51.4 | 1.70E−07 |
| O14686 | 660.3398 | 2 | −4.1 | 34.5 | 1.40E−04 |
| O14686 | 820.7642 | 3 | −1.1 | 36.3 | 1.00E−04 |
| Q8NEZ4 | 712.8812 | 2 | 1.3 | 46.4 | 0.019 |
| Q9Y3A3 | 638.0062 | 3 | 6.2 | 53.9 | 5.60E−06 |
| P26038 | 732.3907 | 3 | 7.2 | 25.9 | 0.0026 |
| Q14149 | 736.3953 | 2 | 0.61 | 24.1 | 0.0051 |
| Q14149 | 720.8905 | 2 | 0.28 | 41.8 | 0.0015 |
| P53985 | 431.5621 | 3 | −9.8 | 26.8 | 0.0017 |
| Q02750 | 415.5408 | 3 | −24 | 28.8 | 0.0062 |
| Q02750 | 709.685 | 3 | 9.6 | 47.9 | 7.20E−07 |
| Q02750 | 752.3787 | 3 | 2.9 | 46.7 | 2.00E−06 |
| O00566 | 466.2746 | 3 | 3.3 | 34.2 | 6.70E−04 |
| O00566 | 652.8626 | 2 | 6.3 | 41.4 | 0.022 |
| Q99549 | 844.3957 | 3 | −0.76 | 35.4 | 1.40E−05 |
| Q99549 | 1028.7731 | 3 | −14 | 23.6 | 3.10E−04 |
| Q99549 | 794.3193 | 2 | −1.6 | 55.5 | 3.40E−05 |
| P49006 | 725.8746 | 2 | −8.1 | 40.6 | 4.00E−04 |
| Q8NHP6 | 602.8095 | 4 | −16 | 37.8 | 9.40E−06 |
| Q86U44 | 709.7824 | 2 | 0.83 | 53.3 | 6.20E−06 |
| P35580 | 792.055 | 3 | −9.6 | 25 | 0.018 |
| P35580 | 634.3247 | 2 | 11 | 29.9 | 0.054 |
| P35749 | 642.3197 | 2 | 7.4 | 27.1 | 0.043 |
| P35579 | 521.604 | 3 | 8.5 | 45.1 | 2.10E−04 |
| P35579 | 627.3053 | 2 | −6.9 | 36.9 | 0.0022 |
| Q13459 | 557.99 | 3 | 1.5 | 34 | 0.0017 |
| O14974 | 627.3053 | 4 | −6.9 | 42.9 | 2.90E−06 |
| O75113 | 688.861 | 2 | 6.6 | 36.9 | 2.00E−04 |
| Q13765 | 858.0908 | 3 | −6.1 | 49.1 | 2.10E−06 |
| Q13765 | 924.4515 | 3 | −24 | 31.4 | 0.0042 |
| Q9BWU0 | 472.7181 | 2 | 7.8 | 26.2 | 2.10E−04 |
| Q9BWU0 | 464.7216 | 2 | 10 | 28.1 | 0.0046 |
| Q9BWU0 | 563.2347 | 2 | 2.6 | 41.1 | 0.0018 |
| A2RRP1 | 938.4413 | 2 | −5.6 | 36.5 | 5.80E−05 |
| A2RRP1 | 569.2801 | 4 | −12 | 39.5 | 5.10E−06 |
| Q69YI7 | 863.7732 | 3 | −5.9 | 50.7 | 1.30E−04 |
| Q9UHQ1 | 726.8673 | 2 | −4.8 | 23.9 | 0.001 |
| Q9UHQ1 | 591.296 | 3 | −0.82 | 40.2 | 4.80E−04 |
| P49321 | 679.5959 | 4 | 1.8 | 38.5 | 3.00E−06 |
| P49321 | 523.927 | 3 | 1.4 | 47.7 | 0.0015 |
| P49321 | 425.2185 | 4 | −4.2 | 39.9 | 6.30E−05 |
| P16333 | 817.8674 | 2 | −1 | 41.4 | 3.50E−07 |
| Q9Y6Q9 | 560.5436 | 4 | 4.2 | 35.2 | 4.00E−05 |
| Q9HCD5 | 456.2735 | 2 | 9.8 | 25.8 | 0.018 |
| Q9HCD5 | 492.2351 | 3 | −8.6 | 42.4 | 6.70E−05 |
| Q14686 | 663.6483 | 3 | −13 | 34.3 | 0.0096 |
| O75376 | 630.8125 | 2 | −6 | 44.6 | 1.20E−04 |
| O75376 | 549.9297 | 3 | −0.67 | 53 | 5.80E−05 |
| O75376 | 596.9484 | 3 | −2.8 | 54.4 | 1.60E−05 |
| O75376 | 721.3387 | 2 | 5 | 30.6 | 2.60E−04 |
| O75376 | 699.316 | 2 | 4.8 | 35.5 | 0.015 |
| O75376 | 636.2653 | 2 | −12 | 32.1 | 0.0085 |
| Q9Y618 | 616.3077 | 2 | −6.8 | 39.8 | 0.0015 |
| Q9Y618 | 903.9988 | 2 | 11 | 42.7 | 1.70E−05 |
| Q92597 | 459.2619 | 3 | −8.7 | 43.3 | 0.0064 |
| Q96SB3 | 889.4941 | 2 | 0.17 | 30.7 | 8.60E−06 |
| Q96PU5 | 635.6574 | 3 | −1.9 | 45.6 | 2.10E−04 |
| Q8NHV4 | 832.9363 | 2 | −8.5 | 38.2 | 0.0049 |
| P46934 | 848.4097 | 3 | 1.9 | 26.7 | 0.016 |
| Q96PY6 | 684.826 | 4 | −9.1 | 33.7 | 0.0076 |
| P51957 | 580.2952 | 2 | −2 | 33.9 | 7.50E−04 |
| Q8TD19 | 770.9013 | 2 | 3.9 | 44.2 | 0.045 |
| Q9H3P2 | 561.8099 | 2 | −2.6 | 24.4 | 7.20E−05 |
| O95644 | 491.7674 | 2 | −4.2 | 37.3 | 0.0064 |
| Q13469 | 747.0483 | 3 | −4 | 48.9 | 1.00E−05 |
| Q00653 | 675.3181 | 2 | 2.1 | 25.5 | 0.0063 |
| Q6P4R8 | 413.7458 | 2 | 7.4 | 25.7 | 0.038 |
| Q6P4R8 | 537.7532 | 4 | −2.4 | 47.4 | 3.00E−07 |
| O14745 | 454.7748 | 2 | 12 | 39.5 | 0.0026 |
| Q86WB0 | 549.7813 | 2 | −2.3 | 33.1 | 0.0039 |
| Q86WB0 | 741.0469 | 3 | 0.66 | 46.8 | 1.20E−05 |
| Q6KC79 | 428.9177 | 3 | 9 | 39.1 | 0.012 |
| Q6KC79 | 493.2756 | 4 | 7 | 24.9 | 0.004 |
| P30414 | 530.8812 | 3 | 13 | 24.9 | 0.002 |
| P46087 | 492.2631 | 2 | −8.1 | 26.2 | 0.045 |
| P46087 | 1155.5475 | 3 | −10 | 52.3 | 5.30E−08 |
| P46087 | 1150.2057 | 3 | −19 | 48.5 | 7.50E−08 |
| P46087 | 794.62 | 5 | 20 | 38 | 1.60E−05 |
| Q9Y2X3 | 594.3311 | 2 | 11 | 29.8 | 0.02 |
| P78316 | 435.8993 | 3 | −5.7 | 33.9 | 0.011 |
| P78316 | 438.8914 | 3 | 21 | 30.9 | 0.019 |
| P55209 | 858.9613 | 2 | −0.83 | 44.8 | 3.60E−07 |
| P55209 | 550.5661 | 4 | −3.2 | 31.1 | 3.30E−04 |
| P55209 | 475.2504 | 3 | 8.3 | 22.8 | 0.0092 |
| Q99733 | 572.7957 | 2 | −0.29 | 37.8 | 4.10E−06 |
| Q99733 | 630.3146 | 3 | 2.4 | 56.1 | 1.00E−08 |
| Q99733 | 813.918 | 4 | 33 | 32 | 1.10E−04 |
| Q99733 | 809.8793 | 4 | −16 | 53.4 | 2.20E−09 |
| Q99733 | 644.9927 | 3 | −3.5 | 46.4 | 1.80E−06 |
| Q49A26 | 556.9489 | 3 | −7 | 35.5 | 0.0015 |
| Q14207 | 458.7825 | 2 | 13 | 25.1 | 0.046 |
| P06748 | 762.0246 | 3 | −15 | 35.4 | 2.40E−05 |
| P06748 | 873.0628 | 3 | −6.9 | 28.7 | 0.004 |
| Q9Y6Y0 | 702.3287 | 3 | −10 | 43.8 | 9.70E−07 |
| P82970 | 628.3083 | 2 | −5.7 | 46.2 | 7.40E−04 |
| Q08J23 | 763.1407 | 4 | −4 | 36.7 | 1.40E−05 |
| Q08J23 | 636.3339 | 5 | −2.5 | 27.9 | 0.002 |
| Q08J23 | 492.2567 | 2 | 8.5 | 24.8 | 0.0075 |
| Q08J23 | 371.2087 | 3 | 17 | 24.1 | 0.013 |
| Q08J23 | 1021.5092 | 2 | 8.1 | 22.2 | 3.20E−04 |
| P49790 | 530.278 | 2 | −9 | 25.6 | 0.033 |
| P80303 | 862.9316 | 2 | −11 | 32.8 | 6.60E−04 |
| P80303 | 684.3105 | 2 | 1.7 | 26.6 | 0.013 |
| Q9H1E3 | 418.7163 | 2 | 12 | 23.2 | 0.08 |
| P19338 | 721.4094 | 2 | −3.3 | 25.2 | 3.20E−04 |
| Q8IVD9 | 456.2354 | 2 | 5.9 | 26.6 | 0.034 |
| Q8IVD9 | 857.4669 | 2 | −0.98 | 39.5 | 3.10E−06 |
| Q8IVD9 | 519.9218 | 3 | 7.6 | 40 | 2.80E−05 |
| Q7Z417 | 635.6724 | 3 | 1.5 | 32.9 | 1.80E−04 |
| Q14980 | 784.3966 | 2 | −14 | 40.5 | 0.0049 |
| Q14980 | 927.2416 | 4 | −11 | 29.4 | 0.001 |
| Q14980 | 602.7781 | 2 | −8.6 | 41.7 | 5.10E−05 |
| Q8NFH3 | 567.9402 | 3 | 2.8 | 38 | 8.00E−05 |
| Q9UKX7 | 639.3153 | 4 | 0.62 | 43.6 | 5.20E−06 |
| Q8N1F7 | 739.3486 | 3 | 7.6 | 38.8 | 1.10E−05 |
| P11177 | 621.2949 | 3 | 5.7 | 33.6 | 0.0033 |
| O75665 | 701.6811 | 3 | −10 | 36.1 | 0.011 |
| Q8WV07 | 495.2563 | 2 | −3.1 | 27.2 | 0.028 |
| Q9BZF1 | 557.611 | 3 | 12 | 33.7 | 0.0014 |
| Q8N6M0 | 700.7099 | 3 | 3.3 | 57.1 | 8.30E−06 |
| Q01804 | 740.3386 | 3 | −6.1 | 28.2 | 4.80E−04 |
| Q8N573 | 698.8929 | 2 | −0.63 | 28.6 | 0.001 |
| Q8N573 | 435.566 | 3 | −0.84 | 33.3 | 6.20E−04 |
| Q6IN85 | 619.801 | 2 | 3.8 | 40.8 | 0.0034 |
| Q8WXI9 | 541.2675 | 2 | 0.45 | 42.8 | 0.0029 |
| P47712 | 652.8033 | 2 | −9.5 | 35.2 | 5.30E−05 |
| Q86U42 | 685.3593 | 2 | −7 | 39.5 | 0.0063 |
| Q86U42 | 532.9647 | 3 | 13 | 55 | 3.60E−05 |
| Q8NC51 | 432.2047 | 3 | 28 | 22.1 | 0.044 |
| Q13153 | 882.9118 | 2 | −12 | 49.2 | 1.60E−05 |
| Q13177 | | | | | |
| Q13177 | 622.8325 | 2 | −4.6 | 39.4 | 4.70E−06 |
| Q8WX93 | 840.421 | 2 | 1.8 | 34.5 | 0.01 |
| Q86W56 | 740.3677 | 3 | 7.7 | 28.4 | 5.70E−04 |
| P09874 | 401.7048 | 2 | −35 | 25.7 | 0.035 |
| P09874 | 465.7711 | 2 | 10 | 33.5 | 0.017 |
| P09874 | 353.5514 | 3 | 18 | 24.1 | 0.0076 |
| P09874 | 586.9405 | 3 | −0.65 | 28.2 | 0.044 |
| Q96IZ0 | 361.2023 | 4 | 8 | 28.4 | 0.0021 |
| P49023 | 467.7274 | 2 | 6.9 | 31.8 | 0.012 |
| P49023 | 612.6551 | 3 | 6.6 | 46.8 | 6.20E−06 |
| P49023 | 771.8785 | 2 | 1.9 | 42.3 | 8.30E−04 |
| Q86U86 | 490.2353 | 3 | −0.87 | 26.6 | 0.045 |
| Q15365 | 1223.6101 | 3 | −5.1 | 30.1 | 0.0016 |
| Q15365 | 643.0097 | 3 | −7.1 | 53.5 | 4.40E−08 |
| Q15365 | 887.1171 | 3 | −5.2 | 49.4 | 2.30E−06 |
| Q15366 | 906.4588 | 3 | 2.1 | 49.9 | 3.90E−06 |
| O94913 | 1111.7555 | 4 | −7.2 | 22.1 | 0.0019 |
| Q15154 | 563.6162 | 3 | −2.6 | 46.3 | 3.40E−05 |
| Q15154 | 764.7111 | 3 | 3.7 | 44 | 4.90E−06 |
| O95613 | 1011.4802 | 3 | 8.1 | 34.6 | 0.0018 |
| Q9BY77 | 748.8864 | 2 | −5.9 | 45.5 | 8.00E−04 |
| O00151 | 893.912 | 2 | 1.7 | 29.3 | 3.20E−04 |
| Q6P996 | 700.8916 | 2 | 1.1 | 41.4 | 6.70E−04 |
| Q13951 | 714.2997 | 2 | −0.56 | 33.9 | 3.80E−07 |
| O94921 | 568.9773 | 3 | 7.1 | 43.1 | 6.10E−04 |
| P00558 | 501.7499 | 2 | 0.087 | 24.7 | 0.018 |
| P00558 | 918.1347 | 3 | −8.1 | 28.2 | 9.60E−04 |
| P00558 | 490.5819 | 3 | 2.8 | 44.4 | 8.50E−06 |
| P00558 | 686.3663 | 3 | −7.1 | 40.4 | 2.80E−05 |
| P00558 | 478.9172 | 3 | 17 | 24.9 | 0.0089 |
| P07205 | | | | | |
| Q8IZ21 | 726.8483 | 2 | 2.8 | 32.1 | 5.70E−04 |
| Q92576 | 838.9101 | 2 | 6.4 | 27.5 | 1.60E−04 |
| Q92576 | 454.8939 | 3 | 18 | 39.9 | 1.50E−06 |
| Q92576 | 941.1218 | 3 | 3.4 | 41.1 | 1.20E−04 |
| Q92576 | 530.9376 | 3 | 1.9 | 43.8 | 1.40E−04 |
| Q6NYC8 | 442.2755 | 2 | 8.7 | 28.1 | 0.012 |
| Q9UBF8 | 766.7201 | 3 | −6.7 | 53.9 | 2.10E−07 |
| Q9UBF8 | 614.3193 | 4 | −2.9 | 24.9 | 0.0093 |
| O75925 | 585.6803 | 3 | 5.7 | 45.4 | 2.20E−06 |
| Q13492 | 515.9336 | 3 | −2.8 | 44 | 4.50E−04 |
| O00562 | 1007.4763 | 2 | −15 | 52.1 | 3.40E−06 |
| O43164 | 659.8461 | 2 | −3 | 29.6 | 0.0021 |
| Q9ULL1 | 921.9333 | 2 | 0.13 | 25.2 | 0.0011 |
| Q99569 | 505.3024 | 2 | −6.4 | 32.2 | 3.60E−05 |
| P19174 | 589.7828 | 2 | 9.1 | 32.8 | 0.039 |
| Q9UL45 | 1347.968 | 3 | −15 | 29.9 | 1.50E−04 |
| Q7Z3K3 | 670.3166 | 2 | −8.7 | 30.9 | 5.30E−04 |
| Q9Y244 | 747.3844 | 3 | −7.6 | 48.5 | 5.30E−07 |
| Q96QC0 | 620.3147 | 3 | 0.11 | 48.1 | 1.80E−05 |
| Q96QC0 | 658.8625 | 2 | −2.5 | 46.5 | 0.015 |
| Q96QC0 | 938.4655 | 3 | −4.8 | 41.7 | 1.10E−04 |
| Q8TF05 | 704.3491 | 3 | −0.9 | 24.2 | 0.0029 |
| P62937 | 556.3095 | 2 | 1 | 22.2 | 0.0042 |
| Q8WUA2 | 857.9648 | 2 | 1.6 | 29.7 | 1.50E−04 |
| O95685 | 442.2522 | 2 | −2.9 | 31 | 0.033 |
| O75400 | 541.7818 | 2 | 8.1 | 46.6 | 0.011 |
| P57071 | 1206.6293 | 2 | 0.34 | 25 | 0.0053 |
| P57071 | 684.3572 | 4 | −6.9 | 44.8 | 4.20E−06 |
| P78527 | 840.0617 | 3 | −0.86 | 25 | 5.70E−04 |
| P07737 | 667.6942 | 3 | −6.2 | 28.8 | 0.0037 |
| P07737 | 528.2513 | 2 | 12 | 26 | 0.025 |
| P07737 | 520.2524 | 2 | 9.5 | 34.9 | 9.30E−04 |
| P07737 | 854.7551 | 3 | −5.8 | 36.1 | 1.00E−04 |
| O60508 | 443.2527 | 2 | −6.7 | 22.9 | 0.0035 |
| O60508 | 572.0596 | 4 | 8.1 | 39 | 5.20E−05 |
| O60508 | 563.2973 | 2 | 13 | 23.6 | 0.0096 |
| O60508 | 465.5632 | 3 | −3.3 | 36.4 | 0.0026 |
| O60508 | 574.2879 | 4 | −4.3 | 47.2 | 1.20E−07 |
| Q8WWY3 | 573.9644 | 3 | 5 | 35.1 | 5.40E−06 |
| Q9ULL5 | 537.7743 | 2 | −1.3 | 23.6 | 0.016 |
| P79522 | 849.7758 | 3 | 8.5 | 33.3 | 0.0019 |
| P62333 | 365.8465 | 3 | 2.9 | 25.1 | 0.0038 |
| P17980 | 465.2692 | 2 | 0.96 | 29.6 | 0.017 |
| P17980 | 678.3531 | 3 | −6.7 | 52 | 9.60E−07 |
| P17980 | 673.0301 | 3 | 6.2 | 53.9 | 2.30E−06 |
| P17980 | 634.8181 | 2 | 1.5 | 22.6 | 0.04 |
| P43686 | 635.799 | 2 | −12 | 53.2 | 6.90E−04 |
| P62195 | 654.986 | 3 | −2 | 46.3 | 5.70E−06 |
| P28066 | 533.939 | 3 | 2.7 | 34 | 7.20E−04 |
| Q8TAA3 | 623.6471 | 3 | 4.8 | 49.9 | 4.00E−04 |
| O14818 | | | | | |
| P20618 | 532.788 | 2 | −4.5 | 25.5 | 9.40E−04 |
| P28070 | 754.3484 | 2 | 0.66 | 45.6 | 0.0018 |
| Q99436 | 493.7846 | 2 | −1 | 22.2 | 0.0037 |
| O00232 | 595.2774 | 2 | −0.74 | 38.2 | 0.0087 |
| Q8NDX1 | 651.809 | 2 | 0.028 | 41.6 | 0.003 |
| Q8NDX1 | 418.2104 | 4 | −5.7 | 39.9 | 8.30E−06 |
| O75475 | 580.7258 | 5 | 4.6 | 43.8 | 5.00E−07 |
| O75475 | 387.8818 | 3 | 3.9 | 31.4 | 0.0051 |
| O75475 | 626.3578 | 2 | 11 | 49.6 | 0.037 |
| P61289 | 468.2449 | 2 | −0.75 | 28.2 | 0.03 |
| P26599 | 577.8366 | 2 | −10 | 41.2 | 5.60E−04 |
| P26599 | 437.604 | 3 | 14 | 39.6 | 9.10E−06 |
| P26599 | 785.8742 | 2 | 3.8 | 47.7 | 1.20E−06 |
| P26599 | 777.8724 | 2 | −1.8 | 44 | 0.0012 |
| P26599 | 777.8709 | 2 | −3.7 | 45.2 | 9.70E−05 |
| P26599 | 769.871 | 2 | −6.9 | 49.1 | 3.10E−05 |
| P26599 | 505.2334 | 2 | −10 | 24.6 | 0.034 |
| Q14761 | 923.0806 | 3 | −11 | 37.9 | 1.10E−05 |
| Q14761 | 1108.5143 | 3 | 2.7 | 35.1 | 5.60E−05 |
| Q14761 | 1063.8215 | 3 | −0.022 | 37.9 | 6.80E−06 |
| P06454 | 654.3372 | 2 | −1.4 | 41 | 2.30E−07 |
| P06454 | 832.4412 | 2 | 0.093 | 35.2 | 5.90E−05 |
| P06454 | 476.2544 | 2 | 2.2 | 29.9 | 0.006 |
| P06454 | 782.9235 | 2 | −2 | 27.6 | 0.0033 |
| P26045 | 564.9375 | 3 | 0.22 | 47.9 | 6.10E−05 |
| P22102 | 834.7403 | 3 | −2 | 57.7 | 1.80E−06 |
| P22102 | 430.2471 | 4 | 29 | 24.1 | 0.0091 |
| P22102 | 591.8156 | 2 | 4.3 | 46 | 0.0065 |
| P22234 | 636.3664 | 2 | −17 | 29.6 | 0.0031 |
| P22234 | 436.2483 | 3 | −4.7 | 24.9 | 0.066 |
| P31939 | 945.0053 | 2 | −12 | 29.8 | 0.041 |
| Q96PZ0 | 765.7245 | 3 | 3.7 | 43.9 | 3.50E−04 |
| Q96PZ0 | 441.9001 | 3 | 5.1 | 39.5 | 9.40E−04 |
| Q96N64 | 876.7814 | 3 | −4.6 | 47.6 | 5.80E−06 |
| P27708 | 974.7468 | 4 | −9.9 | 27.6 | 0.013 |
| Q96PU8 | 716.3922 | 2 | −4.9 | 53.5 | 0.0016 |
| Q5TB80 | 531.7871 | 4 | −1.5 | 53.9 | 1.80E−07 |
| Q2KHR3 | 524.2761 | 2 | −2 | 25.2 | 0.023 |
| Q9Y2K2 | 1107.0348 | 2 | 2.5 | 34.5 | 2.90E−06 |
| Q15032 | 679.3418 | 3 | 9.9 | 38.5 | 1.60E−06 |
| Q15032 | 673.9918 | 3 | −17 | 37.4 | 2.70E−05 |
| P0C7M2 | 557.8447 | 2 | −0.74 | 23.6 | 0.0054 |
| Q32P51 | | | | | |
| P09651 | | | | | |
| P0C7M2 | 481.9217 | 3 | −14 | 29.8 | 0.032 |
| Q32P51 | | | | | |
| P09651 | | | | | |
| P0C7M2 | 503.2672 | 3 | −2.6 | 29.9 | 0.0038 |
| P09651 | | | | | |
| P0C7M2 | 646.0898 | 4 | 2.7 | 28.8 | 0.0037 |
| P09651 | | | | | |
| P0C7M2 | 422.9679 | 4 | 12 | 31.8 | 7.60E−05 |
| P09651 | | | | | |
| O60216 | 1319.9436 | 3 | −27 | 24.4 | 4.60E−04 |
| O60216 | 1439.373 | 3 | −0.97 | 28.2 | 0.0025 |
| O60216 | 1434.0475 | 3 | 3.3 | 28 | 0.0022 |
| O60216 | 681.8575 | 2 | −3.5 | 24.1 | 5.80E−04 |
| Q96JH8 | 745.3844 | 2 | 2.4 | 30.1 | 2.90E−06 |
| Q96JH8 | 539.2765 | 4 | −1.5 | 36.6 | 5.10E−05 |
| P43487 | 636.9929 | 3 | 6.2 | 39 | 4.60E−05 |
| Q15042 | 791.9123 | 2 | 3.9 | 47.8 | 6.20E−04 |
| Q09028 | 456.7423 | 4 | 1.9 | 25.9 | 0.0013 |
| Q16576 | | | | | |
| Q7Z6E9 | 1041.1916 | 3 | 2.9 | 28.1 | 9.90E−04 |
| Q7Z6E9 | 593.3615 | 2 | 18 | 30.4 | 0.004 |
| Q7Z6E9 | 692.8444 | 2 | 8.1 | 28.4 | 6.30E−06 |
| Q7Z6E9 | 559.9359 | 3 | −8.7 | 34.1 | 8.10E−05 |
| Q16576 | 779.3788 | 2 | −2.5 | 29.2 | 8.90E−06 |
| Q16576 | 573.9941 | 4 | −12 | 29.6 | 9.80E−05 |
| Q99708 | 964.4301 | 2 | −12 | 28.1 | 3.80E−05 |
| Q96T37 | 583.2963 | 2 | 13 | 45.6 | 5.30E−05 |
| Q9UPN6 | 739.8526 | 2 | −2 | 30.8 | 5.50E−05 |
| Q9UPN6 | 460.2171 | 3 | 1.5 | 32.4 | 0.0019 |
| P49756 | 716.9792 | 3 | −5.8 | 59.2 | 5.80E−07 |
| Q5T8P6 | 719.011 | 3 | −2.4 | 33.5 | 4.00E−04 |
| Q5T8P6 | 713.675 | 3 | −8.5 | 38.4 | 4.90E−05 |
| Q5T8P6 | 713.8652 | 2 | −2.1 | 22.3 | 7.60E−04 |
| Q5T8P6 | 705.869 | 2 | −0.17 | 22.7 | 2.80E−04 |
| Q9P2N5 | 760.8699 | 2 | −0.91 | 35.9 | 2.70E−07 |
| Q9NW13 | 984.907 | 2 | −6.3 | 50.6 | 3.80E−07 |
| Q96EV2 | 517.2424 | 3 | −3.6 | 38.6 | 2.90E−04 |
| Q14498 | 849.8904 | 2 | −4.9 | 43.8 | 5.00E−07 |
| Q14498 | 890.7576 | 3 | −10 | 45.9 | 2.00E−06 |
| Q9Y5S9 | 601.5095 | 4 | 5.1 | 43.7 | 4.50E−08 |
| Q9Y5S9 | 726.0813 | 4 | −5.9 | 28.1 | 0.0057 |
| Q9Y5S9 | 597.5048 | 4 | −4.9 | 47.7 | 3.70E−10 |
| Q9Y5S9 | 526.4485 | 5 | 9.6 | 30.6 | 9.60E−04 |
| Q9Y5S9 | 962.4462 | 3 | −0.58 | 40.9 | 5.80E−06 |
| Q9Y5S9 | 749.8414 | 2 | −0.93 | 28.1 | 4.30E−04 |
| O43251 | 937.9043 | 2 | 0.75 | 46.3 | 3.90E−05 |
| P49792 | 869.4243 | 2 | 1.4 | 43 | 4.10E−07 |
| P49792 | 606.2963 | 5 | 6.3 | 39.5 | 1.30E−04 |
| P49792 | 764.3483 | 3 | −6.9 | 27.7 | 0.006 |
| P49792 | 453.7201 | 2 | −3 | 23.4 | 0.017 |
| P49792 | 681.5493 | 4 | −3.7 | 34 | 1.70E−05 |
| P49792 | 1157.9086 | 3 | −9.2 | 26.7 | 0.0099 |
| Q68DN6 | | | | | |
| A6NKT7 | | | | | |
| Q7Z3J3 | | | | | |
| Q99666 | | | | | |
| Q53T03 | | | | | |
| O14715 | | | | | |
| P49792 | 984.5243 | 2 | −4.8 | 33.2 | 2.80E−06 |
| A6NKT7 | | | | | |
| Q7Z3J3 | | | | | |
| Q99666 | | | | | |
| Q53T03 | | | | | |
| O14715 | | | | | |
| Q92804 | 566.5941 | 3 | 3 | 47.2 | 1.90E−06 |
| P25800 | 661.3486 | 2 | 5.9 | 24.1 | 0.016 |
| A6NDE4 | 758.0301 | 3 | −3.6 | 22.7 | 0.032 |
| Q15415 | | | | | |
| Q15378 | | | | | |
| P06400 | 666.792 | 2 | −23 | 35.8 | 9.80E−04 |
| P53805 | 632.3271 | 3 | 2.6 | 43.1 | 8.80E−06 |
| Q9P258 | 397.1981 | 2 | 6.2 | 22.4 | 0.013 |
| Q9P258 | 317.1538 | 3 | −40 | 30.6 | 0.014 |
| Q14257 | 808.9093 | 2 | 9.8 | 34.2 | 1.30E−06 |
| Q8IZ40 | 508.2666 | 3 | 3.8 | 39.4 | 0.0019 |
| Q8IZ40 | 774.3639 | 2 | 3 | 55.4 | 5.30E−07 |
| Q8IZ40 | 568.6037 | 3 | −12 | 28.5 | 0.0019 |
| Q8IZ40 | 766.3689 | 2 | 6.2 | 40.2 | 3.10E−05 |
| P54727 | 523.7205 | 2 | −4.6 | 28.4 | 8.70E−04 |
| Q13123 | 818.3831 | 3 | −4.8 | 50.8 | 1.70E−06 |
| Q13123 | 523.2591 | 2 | −8.7 | 22.9 | 0.011 |
| Q13123 | 467.2408 | 3 | −9.9 | 36.2 | 7.00E−04 |
| Q04864 | 717.3198 | 2 | −0.66 | 35.5 | 3.10E−06 |
| Q92900 | 740.0423 | 3 | 6.1 | 62.6 | 3.80E−07 |
| Q96D71 | 861.908 | 2 | −12 | 51.2 | 6.10E−07 |
| Q96D71 | 690.0421 | 3 | −3.3 | 48.9 | 3.90E−07 |
| Q96D71 | 828.6982 | 3 | 0.13 | 59 | 8.00E−08 |
| Q92785 | 591.3139 | 2 | −5.7 | 33.4 | 0.0012 |
| Q92785 | 598.317 | 2 | −4.2 | 35.6 | 0.0012 |
| Q13127 | 476.2368 | 2 | 2.6 | 25.4 | 0.037 |
| P35251 | 691.3596 | 3 | −7.9 | 39.5 | 2.00E−06 |
| P35251 | 467.708 | 2 | 4.3 | 35.5 | 0.003 |
| P35251 | 686.0367 | 3 | 4.9 | 47.4 | 4.90E−05 |
| P35251 | 632.7869 | 2 | −23 | 37.7 | 0.036 |
| Q2KHR2 | 556.6503 | 3 | 2.2 | 29.9 | 0.0067 |
| Q9H0H5 | 638.3082 | 2 | −1.5 | 48.5 | 3.30E−04 |
| Q9H0H5 | 728.8242 | 2 | −5 | 33.2 | 6.60E−04 |
| Q68DN6 | 856.4174 | 2 | 2.5 | 40.1 | 1.30E−07 |
| P0C839 | | | | | |
| A6NKT7 | | | | | |
| Q7Z3J3 | | | | | |
| Q99666 | | | | | |
| Q53T03 | | | | | |
| O14715 | | | | | |
| O43665 | 452.2108 | 3 | −4.1 | 29.8 | 0.0036 |
| O43665 | 519.5628 | 3 | −5.4 | 44.2 | 4.60E−05 |
| P98171 | 698.8462 | 2 | −7.4 | 37 | 4.00E−04 |
| P98171 | 1136.0371 | 2 | −2.9 | 33.7 | 1.40E−06 |
| P42331 | 1154.517 | 3 | 2.3 | 30.6 | 1.50E−05 |
| P42331 | 744.0015 | 3 | 5.2 | 44.6 | 1.90E−04 |
| Q7Z6I6 | 889.9152 | 2 | 3.5 | 27.4 | 3.60E−04 |
| Q7Z6I6 | 1013.5108 | 4 | −3 | 34 | 0.001 |
| Q7Z6I6 | 992.7758 | 3 | −7.1 | 49.4 | 4.70E−08 |
| Q6P4F7 | 935.4164 | 3 | −13 | 29 | 0.0057 |
| P61586 | 640.6616 | 3 | −2.5 | 42.4 | 8.10E−04 |
| P08134 | | | | | |
| Q5UIP0 | 481.2801 | 2 | −3.7 | 33.1 | 0.012 |
| Q5UIP0 | 680.6394 | 3 | 12 | 46.5 | 1.30E−05 |
| O95153 | 838.6621 | 4 | −3.7 | 23.1 | 0.049 |
| O95153 | 529.6074 | 3 | −29 | 28.4 | 0.011 |
| Q06587 | 507.7835 | 2 | 1.9 | 29.4 | 0.022 |
| Q9BRS2 | 551.8251 | 2 | −3.4 | 26.2 | 0.0026 |
| Q9BRS2 | 673.3936 | 2 | 0.097 | 25.7 | 0.0016 |
| Q13546 | 507.9304 | 3 | 15 | 41.1 | 1.30E−06 |
| P31350 | 507.5879 | 3 | −14 | 38.8 | 6.40E−05 |
| P18621 | 830.9908 | 2 | −0.077 | 36.8 | 7.20E−07 |
| P46777 | 891.4249 | 2 | −12 | 50.6 | 9.60E−07 |
| P46777 | 416.2192 | 3 | 0.43 | 31.9 | 4.20E−04 |
| Q8IYW5 | 431.8839 | 3 | 7 | 35 | 3.40E−04 |
| Q63HN8 | 525.2821 | 3 | −0.97 | 32.2 | 0.0011 |
| Q5W0B1 | 608.942 | 3 | −5.1 | 22.5 | 0.031 |
| Q5VTB9 | 586.2893 | 3 | −0.1 | 47.2 | 5.30E−06 |
| Q99942 | 517.239 | 2 | 0.95 | 24.2 | 0.017 |
| Q9H777 | 651.0489 | 4 | −17 | 23.2 | 0.008 |
| Q13151 | 506.5176 | 4 | 4.8 | 47 | 3.70E−06 |
| Q13151 | 501.2954 | 2 | 9.6 | 38.6 | 0.011 |
| Q13151 | 683.329 | 3 | −1.4 | 44.2 | 1.80E−06 |
| Q13151 | 551.7779 | 4 | 6 | 36.5 | 1.20E−05 |
| P22626 | 664.025 | 3 | −2.4 | 31.1 | 0.0099 |
| P22626 | 405.4502 | 4 | 2.1 | 34.1 | 6.60E−06 |
| P22626 | 401.447 | 4 | −9 | 27.1 | 1.20E−04 |
| P22626 | 593.2562 | 2 | −4.1 | 27.7 | 0.0076 |
| P51991 | 518.5953 | 3 | −4.8 | 28.8 | 0.0016 |
| P51991 | 675.4156 | 2 | −1.8 | 26.1 | 4.70E−04 |
| P51991 | 557.8444 | 2 | −1.3 | 27.9 | 5.20E−04 |
| P51991 | 434.4605 | 4 | 2.3 | 30.2 | 2.30E−04 |
| P51991 | 462.9331 | 3 | 7.9 | 33.6 | 7.70E−04 |
| Q13464 | 429.2267 | 2 | −1.7 | 30.1 | 0.049 |
| Q9H6T3 | 378.523 | 3 | −3.6 | 23.8 | 0.012 |
| Q9H6T3 | 926.4674 | 3 | −2.7 | 55.1 | 9.90E−08 |
| Q9H6T3 | 969.1674 | 3 | −0.9 | 47.6 | 2.60E−06 |
| P36954 | 640.8309 | 2 | −6.9 | 34.8 | 2.00E−04 |
| P05423 | 459.2409 | 3 | 3 | 29.9 | 0.015 |
| Q9NVU0 | 678.3157 | 3 | 1.6 | 45.8 | 1.20E−06 |
| Q8TEU7 | 604.7821 | 2 | −0.54 | 36 | 4.80E−05 |
| Q8TEU7 | 596.7848 | 2 | −0.25 | 28.9 | 8.60E−04 |
| Q92766 | 570.2785 | 2 | −7.2 | 29.4 | 0.004 |
| Q8IY81 | 877.8995 | 4 | −10 | 33.8 | 9.60E−06 |
| Q5JTH9 | 788.8457 | 4 | −3 | 41.8 | 8.20E−06 |
| Q5JTH9 | 788.8351 | 4 | −16 | 40.1 | 9.90E−06 |
| Q5JTH9 | 788.8461 | 4 | −2.4 | 36.9 | 1.60E−05 |
| Q5JTH9 | 1046.1287 | 3 | −1.2 | 39.1 | 1.80E−05 |
| Q5JTH9 | 618.2945 | 2 | −6.8 | 29.1 | 3.60E−04 |
| Q14684 | 769.8537 | 4 | −0.7 | 25.8 | 0.004 |
| P60866 | 446.7393 | 4 | 0.93 | 34.2 | 2.70E−05 |
| P62266 | 742.0295 | 3 | 1.6 | 29.7 | 0.0016 |
| P62266 | 784.7221 | 3 | −5.8 | 25.6 | 0.0015 |
| P62857 | 655.3537 | 2 | 0.21 | 45.2 | 0.086 |
| P23396 | 476.2526 | 2 | 8.9 | 31.4 | 0.0036 |
| Q96IZ7 | 549.2897 | 2 | −1.6 | 35.8 | 6.80E−04 |
| Q92541 | 605.2661 | 3 | 10 | 49 | 4.20E−08 |
| Q92541 | 785.8209 | 2 | 1.2 | 40.5 | 3.90E−06 |
| Q92541 | 599.9287 | 3 | 0.35 | 53 | 3.20E−08 |
| Q9NQC3 | 517.2857 | 2 | 17 | 23.3 | 0.0032 |
| Q9NQC3 | 682.331 | 3 | −11 | 47.5 | 2.20E−05 |
| P09234 | 744.8917 | 2 | −3 | 25 | 7.60E−04 |
| P09234 | 509.2383 | 3 | −18 | 38.6 | 2.00E−05 |
| P09661 | 632.8091 | 2 | −4.6 | 34.1 | 0.0041 |
| Q8IZ73 | 827.7338 | 3 | −4.9 | 49.9 | 1.00E−05 |
| P62306 | 524.6212 | 3 | −3.1 | 36.4 | 0.0019 |
| Q8N1F8 | 536.6424 | 3 | 0.61 | 24.6 | 0.029 |
| P55011 | 558.2956 | 3 | −2.6 | 34 | 0.0074 |
| Q96AG3 | 598.0486 | 4 | 12 | 24.9 | 0.013 |
| Q9UHR5 | 572.7506 | 2 | 3 | 36.2 | 2.10E−05 |
| Q15424 | 718.303 | 3 | 0.43 | 39.1 | 2.70E−05 |
| Q15424 | 389.8636 | 3 | 19 | 28.6 | 0.002 |
| Q14151 | | | | | |
| Q15424 | 876.9452 | 4 | 0.7 | 28.5 | 0.0017 |
| Q14151 | | | | | |
| Q15424 | 670.5357 | 5 | −1.7 | 50 | 5.80E−07 |
| Q14151 | | | | | |
| Q15424 | 613.7926 | 4 | 11 | 58.9 | 7.50E−10 |
| Q14151 | | | | | |
| Q15424 | 666.8127 | 2 | −5.4 | 44.1 | 4.80E−04 |
| Q14151 | | | | | |
| Q15424 | 588.7517 | 2 | 0.48 | 45.1 | 0.0033 |
| Q14151 | | | | | |
| Q14151 | 928.9761 | 2 | 3.2 | 38.1 | 6.10E−06 |
| Q14151 | 613.7917 | 2 | 3.2 | 40.8 | 0.0013 |
| Q14151 | 719.6928 | 3 | −0.31 | 49 | 3.40E−06 |
| O43865 | 551.6385 | 3 | −1.8 | 51.6 | 1.20E−06 |
| O43865 | 1054.9309 | 2 | −16 | 45.8 | 3.20E−09 |
| O43865 | 789.3434 | 3 | −4.7 | 32.1 | 1.00E−05 |
| O43865 | 753.8749 | 2 | −5.6 | 31.1 | 0.0013 |
| O43865 | 611.9803 | 3 | −8.2 | 44.8 | 3.90E−06 |
| O43865 | 745.8807 | 2 | −1.2 | 42.4 | 0.006 |
| O43865 | 621.2629 | 2 | −7.7 | 30.5 | 0.012 |
| Q96HN2 | 458.2483 | 2 | −0.03 | 31.2 | 0.052 |
| Q5PRF9 | 785.7272 | 3 | 2.4 | 41.1 | 3.70E−04 |
| Q9UPN7 | 756.6575 | 4 | −6.3 | 47.1 | 3.20E−08 |
| P07602 | 765.863 | 2 | −9.4 | 40.9 | 9.10E−06 |
| P07602 | 663.3343 | 3 | 0.84 | 41.8 | 2.50E−06 |
| P07602 | 603.7409 | 2 | −14 | 38.4 | 0.0013 |
| O75995 | 589.2881 | 2 | −3.9 | 37.5 | 8.30E−05 |
| O75995 | 711.7937 | 2 | −41 | 25.8 | 0.084 |
| Q01826 | 1516.7479 | 3 | 0.93 | 42.1 | 1.70E−07 |
| P43007 | 605.6328 | 3 | 3.2 | 54.9 | 2.70E−05 |
| O15027 | 726.8801 | 2 | −1.7 | 35.8 | 1.40E−05 |
| O15027 | 522.7519 | 2 | 0.83 | 40.2 | 3.00E−04 |
| O95487 | 1144.919 | 3 | 9.5 | 24.5 | 0.023 |
| O14828 | 638.2995 | 2 | −6.9 | 28.6 | 0.027 |
| Q96GD3 | 773.8944 | 2 | −0.26 | 29 | 4.60E−05 |
| O75880 | 1107.1123 | 2 | 4.9 | 24.4 | 0.0014 |
| O75880 | 853.1275 | 3 | −3.2 | 35.1 | 1.60E−04 |
| Q9UIL1 | 637.3021 | 2 | −24 | 32.7 | 0.0016 |
| Q9UIL1 | 510.9325 | 3 | 4.6 | 33.6 | 0.0063 |
| O60524 | 553.6177 | 3 | −6.7 | 36.5 | 0.0019 |
| P55735 | 562.584 | 3 | −2.2 | 32.6 | 2.10E−04 |
| Q12981 | 580.9564 | 3 | 4.3 | 26.8 | 0.046 |
| Q9GZR1 | 703.6621 | 3 | −5.3 | 44.2 | 6.30E−07 |
| Q9UHD8 | 571.7677 | 2 | −20 | 27.9 | 0.018 |
| Q9BYW2 | 402.236 | 3 | 13 | 30.6 | 0.0032 |
| Q9BYW2 | 817.3996 | 3 | 1.9 | 34.7 | 8.20E−05 |
| Q9BYW2 | 883.7687 | 3 | −7.5 | 25.4 | 0.026 |
| Q7Z333 | 787.4313 | 2 | 0.53 | 26.6 | 3.90E−04 |
| Q15637 | 784.8964 | 2 | −13 | 47.5 | 8.40E−06 |
| Q15459 | 356.8125 | 3 | −28 | 24.5 | 9.50E−05 |
| Q15459 | 351.4769 | 3 | −40 | 25 | 1.40E−04 |
| Q15459 | 719.3953 | 3 | −1.7 | 40.2 | 1.30E−04 |
| O75533 | 941.8958 | 2 | −13 | 46.5 | 1.10E−07 |
| Q13435 | 364.2256 | 3 | 14 | 26.9 | 0.055 |
| Q13435 | 898.4512 | 2 | −9 | 46.4 | 1.00E−04 |
| Q13435 | 651.3396 | 3 | −4.1 | 48.5 | 5.60E−06 |
| Q15427 | 618.8253 | 2 | −3 | 38.6 | 5.10E−04 |
| P23246 | 401.2014 | 4 | 1.1 | 30.7 | 0.0017 |
| Q8IX01 | 1310.8661 | 4 | 1.6 | 25.4 | 0.0011 |
| Q8IX01 | 674.3126 | 2 | 0.24 | 45.5 | 0.0011 |
| Q8IX01 | 999.1261 | 3 | −3.4 | 60.5 | 3.30E−10 |
| Q99590 | 835.6921 | 3 | −4.1 | 58.8 | 4.90E−07 |
| Q01130 | 719.8186 | 2 | −0.12 | 28.1 | 1.90E−05 |
| Q01130 | 703.8222 | 2 | −2.2 | 38.3 | 1.90E−04 |
| Q01130 | 545.2738 | 2 | 4.2 | 34.6 | 0.0051 |
| Q01130 | 635.7889 | 2 | −2.2 | 47.8 | 3.50E−04 |
| P84103 | 482.7164 | 2 | 3 | 22.2 | 0.019 |
| Q13243 | 585.2827 | 3 | −5.1 | 49.2 | 9.40E−07 |
| Q13247 | 498.745 | 2 | 8.2 | 28.6 | 0.076 |
| Q5FBB7 | 557.2639 | 3 | 4.1 | 31.6 | 0.0014 |
| Q8N5H7 | 526.9266 | 3 | −2.5 | 40.4 | 0.0014 |
| A0MZ66 | 761.8523 | 2 | −0.94 | 43.8 | 7.40E−07 |
| Q96FS4 | 712.3373 | 2 | −9.7 | 51.2 | 1.70E−06 |
| Q9UIU6 | 928.7876 | 3 | 5.4 | 61.4 | 5.70E−09 |
| P12755 | 668.3472 | 3 | 3.9 | 44.9 | 1.10E−05 |
| Q5T5P2 | 416.5381 | 3 | −1.4 | 29.2 | 0.016 |
| Q9BRT9 | 849.4166 | 3 | −10 | 49.2 | 1.20E−05 |
| Q9H2G2 | 500.2528 | 3 | 7.9 | 28.1 | 2.90E−04 |
| Q14BN4 | 843.8928 | 2 | −13 | 46.8 | 2.00E−05 |
| O95391 | 550.3146 | 2 | 10 | 30.9 | 5.00E−05 |
| O95391 | 681.6812 | 3 | −6.7 | 50.8 | 4.40E−07 |
| O95391 | 543.5403 | 4 | 0.81 | 35.3 | 3.30E−05 |
| O95391 | 742.0353 | 3 | 5.1 | 48.6 | 2.80E−05 |
| O95347 | 618.331 | 3 | 4.6 | 44.6 | 1.30E−06 |
| P51532 | 609.3476 | 2 | −0.79 | 22.4 | 0.01 |
| Q969G3 | 419.8795 | 3 | 1.3 | 32.5 | 0.046 |
| A6NHR9 | 632.291 | 3 | −2.9 | 53.2 | 5.80E−10 |
| Q8TAQ2 | 492.9114 | 3 | −1.3 | 34.4 | 0.0015 |
| Q92925 | 797.8757 | 2 | 6.1 | 42.5 | 3.40E−04 |
| Q5SXM2 | 736.887 | 2 | −13 | 27.7 | 0.057 |
| Q9UMY4 | 914.4595 | 3 | 2.6 | 45.9 | 9.70E−08 |
| Q8TEQ0 | 859.7477 | 3 | −15 | 26.2 | 0.0094 |
| O60749 | 1009.8868 | 3 | 3.2 | 26.1 | 0.0095 |
| O60493 | 599.8294 | 2 | −1.9 | 27.8 | 2.20E−04 |
| Q9UNH7 | 490.7423 | 2 | 9.1 | 28.1 | 0.0089 |
| Q9UNH7 | 426.897 | 3 | 5.8 | 35.2 | 0.031 |
| A7XYQ1 | 711.8476 | 2 | −9.3 | 42.9 | 0.015 |
| P00441 | 828.7504 | 3 | −1.3 | 29 | 0.011 |
| P00441 | 533.6151 | 3 | −0.76 | 55 | 1.10E−05 |
| P18583 | 1296.6627 | 3 | 1 | 28.2 | 0.0013 |
| P18583 | 1096.5876 | 2 | 3.7 | 44.5 | 8.70E−09 |
| P18583 | 754.3801 | 4 | −8.9 | 43 | 1.10E−06 |
| P18583 | 803.7332 | 3 | 15 | 34 | 6.70E−05 |
| P18583 | 626.9766 | 3 | −6.1 | 41.9 | 0.0011 |
| Q9HB58 | 398.5299 | 3 | 14 | 30 | 0.0044 |
| Q9HB58 | 458.8638 | 3 | −13 | 28.9 | 0.0014 |
| P08047 | 1047.0821 | 2 | 5.1 | 44.3 | 2.80E−06 |
| Q02447 | 1236.5731 | 3 | 4.8 | 22.3 | 0.026 |
| Q02447 | 733.3483 | 3 | −7.7 | 58.9 | 3.20E−08 |
| Q86XZ4 | 788.4009 | 2 | −18 | 37.1 | 3.30E−04 |
| Q9UBP0 | 577.758 | 2 | 1.3 | 39 | 0.023 |
| A1X283 | 830.4031 | 2 | −5.8 | 35.4 | 1.60E−06 |
| Q5M775 | 766.3726 | 2 | −2.7 | 38 | 9.80E−04 |
| P19623 | 609.3094 | 2 | −13 | 44.2 | 0.0013 |
| O75934 | 644.9756 | 3 | −0.17 | 51.8 | 6.80E−07 |
| O75940 | 731.3598 | 2 | 0.17 | 33.9 | 4.90E−06 |
| Q8N0X7 | 625.3093 | 2 | 6.9 | 22.1 | 8.50E−04 |
| Q8N0X7 | 506.7756 | 4 | 19 | 26.3 | 0.0069 |
| Q8N0X7 | 538.7899 | 4 | 0.5 | 29.9 | 0.0016 |
| Q9NUQ6 | 482.5808 | 3 | −32 | 27.4 | 0.059 |
| Q7KZ85 | 612.3149 | 2 | −3.4 | 33.8 | 0.0015 |
| Q13813 | 872.4677 | 2 | 0.34 | 31.4 | 1.10E−04 |
| Q13813 | 486.8061 | 2 | −4.1 | 23 | 0.011 |
| O15020 | 605.312 | 3 | −4.8 | 45.7 | 1.50E−04 |
| O15042 | 910.7951 | 3 | −14 | 26.2 | 0.091 |
| O15042 | 967.4972 | 2 | −32 | 46.4 | 1.30E−05 |
| O15042 | 828.7499 | 3 | 0.01 | 38 | 2.70E−05 |
| O15042 | 653.8394 | 4 | 2.1 | 38 | 1.30E−04 |
| O15042 | 608.8057 | 2 | −1.1 | 35.2 | 1.80E−07 |
| O15042 | 448.9081 | 3 | 6.8 | 41.1 | 2.60E−07 |
| O15042 | 519.029 | 4 | 3.9 | 48.2 | 7.40E−08 |
| Q6ZRS2 | 788.3406 | 3 | −5.5 | 45.7 | 2.60E−06 |
| Q6ZRS2 | 848.6655 | 3 | −30 | 41.6 | 4.20E−08 |
| P12931 | 860.4475 | 2 | −2.8 | 26.5 | 1.70E−04 |
| Q8NEF9 | 531.2961 | 2 | 1.1 | 27.9 | 0.0016 |
| Q8NEF9 | 478.7739 | 4 | −0.65 | 22.5 | 0.085 |
| Q9UHB9 | 585.5641 | 4 | 17 | 49.3 | 1.30E−07 |
| Q9UHB9 | 518.2524 | 3 | 12 | 38.7 | 0.004 |
| Q96SB4 | 1439.3376 | 3 | 5.3 | 28 | 2.60E−04 |
| Q9UQ35 | 489.739 | 2 | 7 | 28.9 | 0.0047 |
| Q9UQ35 | 523.7805 | 2 | −5 | 36.5 | 0.0018 |
| Q9UQ35 | 433.8676 | 3 | −15 | 22.8 | 0.078 |
| O60232 | 639.9947 | 3 | −4.3 | 55.7 | 6.90E−06 |
| Q9BWW4 | 1146.555 | 2 | 4.5 | 22.9 | 0.0016 |
| Q9NQ55 | 831.9291 | 2 | −2.9 | 41 | 8.60E−08 |
| P28290 | 769.4176 | 2 | 1.3 | 41.8 | 0.0041 |
| Q76I76 | 792.3868 | 3 | −0.59 | 30.4 | 0.0021 |
| Q08945 | 912.4597 | 2 | −2.8 | 49.6 | 8.00E−06 |
| Q9ULZ2 | 640.9686 | 3 | −3.6 | 55.6 | 1.60E−05 |
| Q9ULZ2 | 635.637 | 3 | −3.7 | 57.2 | 3.90E−05 |
| O94804 | 1440.6692 | 4 | 1.1 | 25.8 | 2.10E−04 |
| Q9Y6E0 | 919.9206 | 2 | −18 | 54.6 | 3.10E−08 |
| Q9Y6E0 | 609.8011 | 4 | 1.4 | 32.3 | 0.0096 |
| Q9UEW8 | 799.3599 | 2 | 16 | 55.5 | 4.90E−04 |
| Q13043 | 1230.8882 | 3 | 1.3 | 33.7 | 2.40E−04 |
| Q13043 | 962.4368 | 4 | −5.5 | 22.6 | 0.025 |
| Q13043 | 801.4111 | 5 | 45 | 35 | 8.50E−05 |
| O43815 | 483.2445 | 2 | 1.9 | 32.5 | 0.0058 |
| O43815 | 694.8362 | 2 | −2.9 | 48.2 | 9.90E−04 |
| O43815 | 615.3103 | 3 | −1.1 | 43.5 | 4.70E−04 |
| O43815 | 493.7484 | 4 | −21 | 28.1 | 0.0056 |
| O60499 | 562.9101 | 3 | −1.1 | 23.4 | 3.30E−04 |
| O60499 | 550.2824 | 2 | 18 | 32.8 | 0.019 |
| Q86Y82 | 645.6447 | 3 | 5.4 | 62.6 | 2.30E−06 |
| P56962 | 585.3108 | 4 | 9.3 | 30.6 | 1.00E−04 |
| P56962 | 697.6675 | 3 | −5.2 | 53.4 | 8.10E−05 |
| O15400 | 905.4229 | 3 | −22 | 66.6 | 1.80E−08 |
| Q9Y2Z0 | 920.9676 | 2 | −14 | 26.6 | 0.0026 |
| Q96A49 | 537.5916 | 3 | 0.68 | 43.1 | 2.60E−04 |
| P07814 | 702.9106 | 2 | 3.1 | 52.2 | 0.0046 |
| O95926 | 567.9626 | 3 | 4.7 | 25.8 | 0.0053 |
| O95926 | 619.9912 | 3 | −4.1 | 51.6 | 2.40E−07 |
| P41250 | 716.8691 | 2 | −8.5 | 52.1 | 6.30E−05 |
| Q92797 | 524.2259 | 2 | 3.2 | 31.3 | 0.0031 |
| O43776 | 971.4463 | 2 | −0.63 | 31 | 8.20E−05 |
| Q8NF91 | 610.962 | 3 | 3.1 | 50.1 | 5.70E−07 |
| Q8WXH0 | 601.2927 | 2 | −2.1 | 44.7 | 2.40E−04 |
| P23381 | 775.8948 | 2 | −1.7 | 48.5 | 1.10E−05 |
| Q9NUM4 | 580.2535 | 2 | −4.4 | 37.4 | 3.10E−04 |
| Q9NUM4 | 572.2618 | 2 | 5.6 | 33.8 | 0.0046 |
| Q9BVX2 | 913.4464 | 3 | −20 | 31.1 | 0.0016 |
| P29083 | 605.0193 | 4 | 5.4 | 35.1 | 1.90E−04 |
| P29083 | 601.0209 | 4 | 5.9 | 44.9 | 1.80E−06 |
| P35269 | 1007.3983 | 2 | 1.3 | 55.5 | 1.30E−08 |
| P35269 | 999.4013 | 2 | 1.8 | 52.8 | 2.30E−07 |
| O75410 | 669.3194 | 2 | −1.3 | 24.8 | 3.40E−05 |
| O75410 | 635.2991 | 3 | 0.77 | 34.2 | 3.00E−05 |
| O75410 | 379.4883 | 3 | −31 | 22.4 | 0.0045 |
| O95359 | 459.5494 | 3 | −2.8 | 24.8 | 0.021 |
| Q9Y6A5 | 921.7929 | 3 | 5.9 | 31.8 | 3.90E−04 |
| Q9Y6A5 | 750.3785 | 2 | −2.1 | 34.1 | 0.0043 |
| Q96BN2 | 504.0201 | 4 | 1.7 | 31.8 | 3.90E−04 |
| Q15544 | 829.8834 | 2 | −9.7 | 39.6 | 3.50E−05 |
| Q15545 | 1032.492 | 2 | −2 | 30.2 | 0.0038 |
| Q71U36 | 669.1185 | 5 | 4.2 | 56.2 | 1.60E−08 |
| P68363 | | | | | |
| Q9BQE3 | | | | | |
| Q13748 | | | | | |
| Q6PEY2 | | | | | |
| Q71U36 | 665.9205 | 5 | 5.7 | 62.1 | 1.10E−09 |
| P68363 | | | | | |
| Q9BQE3 | | | | | |
| Q13748 | | | | | |
| Q6PEY2 | | | | | |
| Q71U36 | 946.0852 | 3 | −11 | 51.1 | 1.30E−08 |
| P68363 | | | | | |
| Q9BQE3 | | | | | |
| Q13748 | | | | | |
| Q6PEY2 | | | | | |
| Q71U36 | 492.5025 | 4 | 5.7 | 42.9 | 6.80E−06 |
| P68363 | | | | | |
| Q9BQE3 | | | | | |
| Q13748 | | | | | |
| Q6PEY2 | | | | | |
| Q71U36 | 514.7216 | 2 | 0.51 | 34.3 | 0.035 |
| P68363 | | | | | |
| Q9BQE3 | | | | | |
| Q13748 | | | | | |
| Q6PEY2 | | | | | |
| P68366 | | | | | |
| Q71U36 | 706.6535 | 3 | −5 | 27.8 | 0.0062 |
| P68363 | | | | | |
| Q9BQE3 | | | | | |
| Q13748 | | | | | |
| Q6PEY2 | | | | | |
| P68366 | | | | | |
| Q9NY65 | | | | | |
| Q71U36 | 744.7286 | 3 | 2.4 | 36.4 | 9.50E−06 |
| P68363 | | | | | |
| Q9BQE3 | | | | | |
| Q13748 | | | | | |
| Q6PEY2 | | | | | |
| P68366 | | | | | |
| Q9NY65 | | | | | |
| Q13885 | 334.2091 | 3 | −2.1 | 22 | 0.071 |
| Q9BVA1 | | | | | |
| P68371 | | | | | |
| Q13509 | | | | | |
| P07437 | | | | | |
| Q13885 | 436.7664 | 2 | 6.4 | 30.8 | 0.087 |
| Q9BVA1 | | | | | |
| P68371 | | | | | |
| Q13509 | | | | | |
| P07437 | | | | | |
| P68371 | 859.4191 | 5 | −15 | 41.1 | 1.90E−05 |
| P07437 | | | | | |
| Q15814 | 658.03 | 3 | 0.2 | 39.1 | 4.50E−07 |
| O60343 | 763.0459 | 3 | −3.5 | 55.2 | 3.30E−05 |
| O60343 | 672.0145 | 3 | −3.1 | 50.5 | 4.90E−05 |
| Q9BZK7 | 586.3129 | 3 | −20 | 32.1 | 0.0085 |
| Q9BZK7 | 691.336 | 2 | −9.6 | 43.6 | 0.016 |
| O60907 | | | | | |
| Q9BZK7 | 609.805 | 2 | 2.2 | 40.9 | 0.0013 |
| Q9BQ87 | | | | | |
| O60907 | 598.2951 | 2 | −1 | 39.3 | 0.032 |
| P23193 | 603.7908 | 2 | −3.8 | 27.3 | 0.0051 |
| Q9UGU0 | 1006.0216 | 2 | −1.5 | 48.2 | 3.60E−09 |
| Q9UGU0 | 954.2312 | 4 | −2.5 | 22.6 | 0.02 |
| Q9UGU0 | 548.7724 | 4 | −4 | 32.2 | 0.0013 |
| Q13428 | 935.4953 | 2 | −0.91 | 37 | 3.50E−07 |
| Q13428 | 927.4999 | 2 | 1.3 | 37.6 | 1.50E−07 |
| Q13428 | 958.0103 | 2 | 0.16 | 32.3 | 1.80E−06 |
| P50991 | 904.4691 | 3 | −13 | 38.6 | 6.50E−05 |
| P50991 | 525.2248 | 2 | 6.3 | 34.3 | 0.002 |
| P50991 | 517.2261 | 2 | 4 | 31.5 | 0.017 |
| P48643 | 872.7516 | 3 | −9.3 | 36 | 4.90E−04 |
| P48643 | 652.3692 | 3 | −6.7 | 48.5 | 7.30E−07 |
| P40227 | 676.3702 | 3 | 15 | 31.4 | 3.40E−04 |
| P13693 | 545.2834 | 2 | −1.3 | 29.7 | 0.0049 |
| O60522 | 686.3245 | 4 | 7.1 | 24.2 | 0.079 |
| Q8IWB9 | 575.7741 | 2 | −6.8 | 30.8 | 0.0014 |
| Q8IWB9 | 858.9738 | 2 | −7.4 | 24.2 | 4.90E−04 |
| Q00403 | 703.8401 | 2 | 7.7 | 31.8 | 0.0032 |
| Q00403 | 695.8365 | 2 | −0.95 | 37.1 | 0.029 |
| Q9NZI6 | 575.849 | 2 | 3.2 | 36.2 | 0.073 |
| Q12800 | | | | | |
| Q9NZI7 | | | | | |
| Q92664 | 697.7161 | 3 | −0.33 | 44.4 | 6.10E−05 |
| Q04206 | 721.3227 | 2 | −2.5 | 35.6 | 4.20E−06 |
| Q96RS0 | 428.8935 | 3 | 9 | 27.2 | 0.0093 |
| Q96RS0 | 720.8799 | 2 | −2 | 30.9 | 4.60E−05 |
| Q96RS0 | 709.9974 | 3 | −2.8 | 24.1 | 0.012 |
| Q86V81 | 715.8326 | 2 | 2.3 | 46.5 | 4.30E−07 |
| Q13769 | 440.2166 | 2 | 6.1 | 25.8 | 0.05 |
| P52888 | 687.3451 | 2 | −0.95 | 35.6 | 3.60E−05 |
| O15164 | 664.6242 | 3 | −5.5 | 36.4 | 1.30E−04 |
| Q13263 | 1426.8933 | 3 | 1.10E−04 | 38.5 | 3.70E−08 |
| Q13263 | 824.3788 | 2 | 2.4 | 42.8 | 4.30E−09 |
| Q13263 | 1010.0201 | 2 | −21 | 37.5 | 4.00E−04 |
| Q13263 | 577.788 | 2 | 0.019 | 43.1 | 0.002 |
| Q13263 | 456.2436 | 2 | −3.8 | 23.4 | 0.061 |
| Q9UNS1 | 972.4909 | 2 | 1.9 | 35.7 | 9.10E−06 |
| Q9BSI4 | 1033.9965 | 2 | −3.2 | 25.8 | 1.80E−04 |
| Q9BSI4 | 1026.0031 | 2 | 0.72 | 41.1 | 1.30E−05 |
| Q86UE8 | 934.4821 | 3 | −7.8 | 22.9 | 0.044 |
| Q9H0V1 | 731.0532 | 3 | −2.6 | 29.4 | 0.012 |
| Q6ZVM7 | 402.2346 | 3 | 7.9 | 22.8 | 0.0059 |
| Q9BVT8 | 608.9508 | 3 | −9.2 | 36.5 | 4.60E−04 |
| Q9BVT8 | 603.6198 | 3 | −8.2 | 44 | 4.50E−04 |
| Q8NFZ5 | 478.9161 | 3 | 7.9 | 44.1 | 9.40E−04 |
| Q8NDV7 | 782.8917 | 2 | −3.4 | 27.9 | 7.00E−04 |
| Q8NDV7 | 620.07 | 4 | 8.1 | 23.2 | 0.0096 |
| Q96GM8 | 1008.9917 | 2 | 2 | 42.9 | 1.30E−07 |
| Q96GM8 | 762.3608 | 2 | −13 | 32.7 | 6.70E−05 |
| Q5JTV8 | 938.495 | 2 | 3.1 | 37.6 | 3.00E−07 |
| Q5JTV8 | 448.9175 | 3 | 11 | 35.3 | 4.20E−05 |
| Q5JTV8 | 428.2089 | 3 | −12 | 34.5 | 8.60E−04 |
| Q9H0E2 | 620.6697 | 3 | 12 | 44.5 | 4.60E−07 |
| O60784 | 464.7612 | 2 | −6.4 | 38.2 | 0.08 |
| O60784 | 1207.2751 | 3 | −3.6 | 26 | 0.0021 |
| O60784 | 1360.34 | 3 | −3.7 | 28.4 | 6.00E−05 |
| O60784 | 482.5728 | 3 | −12 | 38.5 | 6.80E−04 |
| O60784 | 477.2509 | 3 | 8.7 | 34.5 | 0.0015 |
| Q02880 | 738.8719 | 2 | −0.28 | 41.4 | 0.0032 |
| Q12888 | 656.8251 | 4 | −0.49 | 46 | 1.30E−06 |
| Q12888 | 839.9387 | 4 | 30 | 34.3 | 3.70E−04 |
| Q12888 | 721.3541 | 2 | −4.2 | 52.6 | 0.023 |
| Q12888 | 507.7201 | 2 | 1.9 | 23.5 | 0.024 |
| Q12888 | 667.3603 | 2 | 1.7 | 30.1 | 0.0082 |
| O43399 | 721.8639 | 2 | −2 | 26.3 | 2.70E−04 |
| Q5T0D9 | 1006.5086 | 3 | −9.5 | 32.5 | 3.30E−04 |
| Q5T0D9 | 913.2612 | 5 | −16 | 22 | 0.07 |
| P12270 | 1124.0409 | 2 | 2.1 | 33.2 | 1.30E−05 |
| P12270 | 629.3242 | 3 | 1.3 | 60.4 | 5.40E−08 |
| Q9Y2W1 | 663.6706 | 3 | −3.2 | 53 | 4.60E−08 |
| Q15633 | 672.3055 | 3 | −4.1 | 48.2 | 1.30E−07 |
| Q96PN7 | 763.6944 | 3 | −13 | 32.8 | 1.80E−05 |
| Q9UPN9 | 583.8284 | 2 | −2.8 | 28.9 | 4.30E−04 |
| Q9UPN9 | 815.7195 | 3 | 2.2 | 28.2 | 0.0058 |
| Q9UPN9 | 810.3852 | 3 | −1.1 | 34.6 | 2.90E−04 |
| Q15650 | 557.6242 | 3 | −3.3 | 39.4 | 6.60E−04 |
| Q15650 | 848.3895 | 2 | 4 | 45.1 | 2.00E−04 |
| Q7Z2T5 | 1096.5593 | 3 | −4.9 | 54.4 | 3.00E−10 |
| Q9Y2L5 | 473.2404 | 3 | 12 | 43.7 | 0.0099 |
| Q92574 | 736.869 | 2 | −7.2 | 42.8 | 4.60E−04 |
| Q92574 | 656.089 | 4 | −0.31 | 26 | 0.0095 |
| Q92574 | 827.393 | 2 | −0.13 | 50 | 1.50E−04 |
| Q2NL82 | 604.2764 | 2 | −4.5 | 39.1 | 1.10E−06 |
| Q2NL82 | 560.285 | 3 | 0.83 | 48 | 3.30E−08 |
| Q2NL82 | 596.2769 | 2 | −8.1 | 39.6 | 3.20E−05 |
| Q2NL82 | 560.2876 | 3 | 5.6 | 37 | 1.10E−06 |
| Q2NL82 | 609.9619 | 3 | −8.5 | 42.7 | 8.30E−05 |
| Q99614 | 661.2932 | 3 | −8 | 41.5 | 2.00E−06 |
| O95801 | 612.3082 | 2 | −3 | 29.2 | 1.00E−04 |
| Q9UNY4 | 711.4294 | 2 | −5 | 22.4 | 0.0033 |
| Q9UNY4 | 517.3247 | 3 | 3.9 | 29 | 0.0014 |
| P63313 | 624.2846 | 2 | 0.51 | 41 | 0.0033 |
| P04818 | 426.731 | 2 | −3.2 | 23 | 0.0056 |
| P25490 | 1025.0097 | 4 | −13 | 33.5 | 3.20E−05 |
| Q13432 | 429.9161 | 3 | 4.5 | 42.9 | 2.20E−04 |
| A6NIH7 | 668.8739 | 4 | 0.24 | 40 | 2.50E−06 |
| P26368 | 692.6958 | 3 | −3.2 | 49.2 | 1.10E−07 |
| P26368 | 687.3638 | 3 | −3.7 | 58.5 | 4.20E−08 |
| Q3KQV9 | 1094.0704 | 2 | 1.9 | 43.5 | 2.00E−08 |
| Q13838 | 511.2687 | 2 | −1.2 | 32.2 | 0.049 |
| P22314 | 658.6626 | 3 | −0.22 | 36.1 | 9.70E−05 |
| Q8TBC4 | 715.3295 | 3 | 0.66 | 28.7 | 0.0019 |
| Q8TBC4 | 758.7929 | 2 | 1.8 | 48.6 | 7.10E−07 |
| Q5T6F2 | 759.3829 | 2 | −0.63 | 22.5 | 3.60E−04 |
| Q5T6F2 | 570.7653 | 2 | 2.8 | 30.8 | 4.90E−06 |
| Q5T6F2 | 597.2897 | 2 | 3 | 33 | 0.0015 |
| Q9C0C9 | 1065.1611 | 3 | −9.6 | 28.9 | 7.90E−04 |
| Q9C0C9 | 1059.8263 | 3 | −13 | 38.6 | 1.60E−04 |
| Q9C0C9 | 880.6616 | 4 | −13 | 26.6 | 0.0033 |
| Q9C0C9 | 1302.6199 | 3 | 5.3 | 30.6 | 0.0012 |
| Q9C0C9 | 420.5742 | 3 | 7 | 24.5 | 7.50E−04 |
| O14562 | 1090.4749 | 4 | −15 | 42.2 | 4.40E−08 |
| Q9NPG3 | 1143.0427 | 2 | −18 | 44.7 | 3.20E−07 |
| Q14694 | 753.6938 | 3 | −8 | 31.3 | 2.60E−04 |
| Q14694 | 458.2583 | 2 | 9.5 | 38 | 0.021 |
| Q14694 | 701.3464 | 3 | −7.4 | 39.1 | 3.10E−05 |
| P54578 | 513.7778 | 2 | 1.6 | 32 | 0.018 |
| P54578 | 622.2935 | 2 | −0.35 | 43.6 | 3.30E−05 |
| O94966 | 576.2896 | 3 | 2 | 28.2 | 0.0018 |
| Q14157 | 607.8398 | 2 | 6.9 | 22 | 0.0015 |
| Q14157 | 791.3962 | 3 | −15 | 48.8 | 7.20E−07 |
| Q14157 | 830.3892 | 2 | −9 | 31.8 | 2.80E−04 |
| Q70CQ2 | 463.2114 | 2 | 4 | 26.1 | 0.028 |
| Q70CQ2 | 706.8328 | 2 | 5.3 | 27.7 | 4.20E−04 |
| Q9P275 | 540.8051 | 2 | 8.6 | 27.9 | 0.016 |
| Q9H9J4 | 445.7561 | 2 | 12 | 37.4 | 8.80E−04 |
| P45974 | 625.7867 | 2 | −1.7 | 45 | 2.30E−06 |
| P45974 | 617.7907 | 2 | 0.68 | 30 | 3.50E−05 |
| P45974 | 480.615 | 3 | 6.2 | 32.1 | 1.80E−04 |
| P45974 | 623.837 | 2 | −9.9 | 36 | 0.033 |
| P45974 | 675.3514 | 2 | 0.65 | 33.6 | 9.00E−05 |
| Q93009 | 659.9335 | 3 | −0.98 | 45.6 | 2.80E−07 |
| Q93009 | 720.2814 | 3 | 2 | 50.9 | 7.00E−07 |
| Q9UMX0 | 870.4346 | 2 | 3.5 | 29.1 | 1.30E−04 |
| Q5T4S7 | 390.8701 | 3 | 8.1 | 35.6 | 1.60E−05 |
| O94888 | 540.5906 | 3 | −15 | 42.6 | 3.90E−04 |
| O94888 | 601.534 | 4 | −23 | 30 | 6.90E−04 |
| O94888 | 717.3751 | 2 | 11 | 34.3 | 1.10E−05 |
| Q16851 | 692.8494 | 2 | 4.3 | 54.5 | 0.063 |
| A0JNW5 | 859.4135 | 3 | −1.4 | 26.5 | 0.0025 |
| Q96T88 | 747.3409 | 3 | −4.2 | 42 | 2.50E−06 |
| Q96T88 | 797.411 | 4 | 48 | 60.3 | 1.00E−08 |
| Q86UX7 | 626.3471 | 2 | 3.1 | 42.8 | 0.011 |
| Q86UX7 | 443.9826 | 4 | −26 | 38.1 | 4.10E−04 |
| Q9NZ43 | 650.2803 | 2 | 0.97 | 33.5 | 6.70E−04 |
| Q15853 | 1464.7474 | 3 | −4.7 | 35.2 | 2.50E−05 |
| O60763 | 567.7412 | 2 | −4.8 | 25.5 | 0.0023 |
| P46939 | 634.8724 | 2 | 4.3 | 25.6 | 0.0021 |
| P63027 | 577.2989 | 3 | 25 | 30.6 | 4.10E−04 |
| Q15836 | | | | | |
| Q9Y5K8 | 630.3138 | 2 | −5 | 31.2 | 0.0048 |
| P08670 | 454.2513 | 2 | 7.7 | 27.6 | 0.019 |
| P08670 | 639.8444 | 2 | 1.1 | 23.8 | 2.50E−04 |
| P08670 | 651.8523 | 2 | −2.9 | 30.6 | 1.00E−04 |
| P08670 | 720.8728 | 2 | 0.96 | 41.1 | 1.10E−05 |
| P08670 | 604.6469 | 3 | −1.4 | 65 | 9.00E−08 |
| P08670 | 705.0226 | 3 | −1.3 | 52.9 | 4.50E−07 |
| P08670 | 376.4564 | 4 | 5.1 | 31.5 | 3.40E−04 |
| P08670 | 545.6313 | 3 | 2.9 | 37.2 | 2.90E−05 |
| P08670 | 502.0231 | 4 | −0.35 | 27.4 | 0.0011 |
| P08670 | 474.2526 | 3 | −25 | 44 | 3.20E−04 |
| P08670 | 448.502 | 4 | 5.7 | 27.1 | 0.0059 |
| Q5THJ4 | 679.3378 | 2 | −4.6 | 31.2 | 0.018 |
| Q9UN37 | 588.9353 | 3 | −0.85 | 47.2 | 5.10E−04 |
| Q9UN37 | 640.9725 | 3 | 4.7 | 27.1 | 0.0062 |
| Q99986 | 386.86 | 3 | 0.44 | 33.9 | 0.0011 |
| Q7Z5K2 | 476.2366 | 2 | 10 | 24.1 | 0.0055 |
| Q92558 | 779.7277 | 3 | 11 | 54 | 1.30E−05 |
| Q9Y6W5 | 1083.515 | 4 | 16 | 41.1 | 5.20E−06 |
| Q9Y6W5 | 633.0053 | 3 | −2.7 | 45.2 | 4.30E−05 |
| A8K0Z3 | 984.5248 | 4 | −17 | 52.7 | 2.40E−07 |
| Q9C0J8 | 435.7081 | 4 | 6.3 | 24.8 | 0.0035 |
| Q5JSH3 | 972.0395 | 2 | 8.3 | 37.9 | 8.50E−06 |
| Q9H6Y2 | 438.7174 | 2 | 3.7 | 27.6 | 0.027 |
| O43379 | 1109.2663 | 3 | −6 | 37.5 | 1.50E−05 |
| Q96MX6 | 912.9972 | 2 | 3.2 | 33 | 1.10E−06 |
| O76024 | 496.7763 | 2 | −7.2 | 31.2 | 0.0013 |
| O76024 | 730.6953 | 3 | −1.5 | 35.3 | 8.80E−04 |
| O43516 | 608.3255 | 4 | −3.5 | 45.9 | 1.80E−08 |
| Q9H4A3 | 707.815 | 4 | 4.7 | 34.3 | 2.70E−05 |
| Q9H4A3 | 1167.0025 | 2 | −3.4 | 50.4 | 3.30E−11 |
| Q9H4A3 | 563.2531 | 5 | 2.4 | 41 | 7.00E−07 |
| Q9H4A3 | 414.6975 | 4 | −5.4 | 37 | 3.30E−06 |
| Q9H4A3 | 932.4238 | 2 | 2.5 | 58 | 2.30E−06 |
| Q9H4A3 | 838.7054 | 3 | 23 | 56.8 | 1.00E−07 |
| Q96S55 | 622.6505 | 3 | 0.23 | 29.8 | 0.0039 |
| Q6AWC2 | 443.279 | 2 | −0.92 | 25 | 0.011 |
| P23025 | 747.3813 | 3 | −12 | 60 | 1.40E−07 |
| P46937 | 682.3796 | 2 | 5.7 | 23.4 | 6.30E−04 |
| P67809 | 855.4151 | 3 | 3.3 | 44.5 | 1.10E−06 |
| P67809 | 673.8279 | 4 | −10 | 47.1 | 1.30E−08 |
| P67809 | 863.0819 | 3 | −27 | 48.6 | 7.20E−08 |
| Q9GZM5 | 826.8774 | 2 | 0.15 | 32.9 | 6.80E−05 |
| Q6ZSR9 | 802.416 | 2 | −14 | 31.1 | 4.50E−04 |
| Q6ZSR9 | 585.8135 | 2 | −9.4 | 30 | 0.0073 |
| A8MX80 | 652.3391 | 3 | 0.68 | 22.5 | 0.028 |
| Q9H6S0 | 895.3569 | 2 | −0.24 | 53.6 | 4.40E−07 |
| Q9BYJ9 | 679.841 | 2 | 3.5 | 26.3 | 9.90E−05 |
| Q9BYJ9 | 726.6064 | 4 | 0.45 | 22.8 | 0.024 |
| Q9BYJ9 | 718.6071 | 4 | −2.2 | 39.8 | 9.40E−06 |
| Q9Y5A9 | 669.3386 | 4 | −8.1 | 46.5 | 3.20E−07 |
| Q9Y5A9 | 682.8468 | 2 | 4.3 | 30 | 7.20E−05 |
| Q9Y5A9 | 955.1275 | 3 | −14 | 62.1 | 4.20E−09 |
| Q9Y5A9 | 1012.2638 | 4 | 0.3 | 28.2 | 0.0086 |
| Q9Y5A9 | 857.4025 | 3 | −7.4 | 69.2 | 6.50E−09 |
| Q7Z739 | 926.4589 | 3 | −19 | 48 | 9.90E−07 |
| Q7Z739 | 722.8388 | 2 | −0.035 | 33.7 | 0.0014 |
| P43403 | 488.2466 | 2 | −3.6 | 33.3 | 7.70E−04 |
| Q8NCN2 | 730.3552 | 2 | 0.59 | 30.5 | 2.40E−04 |
| Q8NCP5 | 839.407 | 2 | −2.6 | 22.4 | 1.60E−04 |
| O75152 | 668.407 | 2 | −2.9 | 27.7 | 5.00E−05 |
| O75152 | 620.8188 | 4 | 2.3 | 40.1 | 2.20E−06 |
| Q9UPT8 | 759.8306 | 2 | −8.4 | 51 | 2.40E−05 |
| Q9UPT8 | 674.1222 | 4 | 32 | 35.5 | 6.20E−05 |
| Q5T200 | 497.2648 | 3 | −2.2 | 24.9 | 0.016 |
| Q5T200 | 505.5844 | 3 | 13 | 28.2 | 0.0013 |
| Q6PJT7 | 1304.925 | 3 | −1.4 | 27.4 | 3.80E−04 |
| Q7Z2W4 | 553.298 | 2 | 3.6 | 30.3 | 9.60E−05 |
| Q7Z2W4 | 663.3275 | 2 | −5 | 45.2 | 2.00E−04 |
| Q86VM9 | 741.0172 | 3 | 1.5 | 24.5 | 0.022 |
| Q9C0B9 | 620.3225 | 2 | 4 | 46.2 | 2.10E−05 |
| Q6NZY4 | 708.8519 | 2 | −0.04 | 36.9 | 6.20E−04 |
| P37275 | 869.06 | 3 | −2.6 | 44.5 | 5.20E−07 |
| O43829 | 785.3593 | 3 | −0.25 | 51.6 | 1.90E−07 |
| O43829 | 851.0337 | 3 | −3.8 | 57.4 | 4.40E−08 |
| Q6FIF0 | 1121.534 | 3 | 4.7 | 28.7 | 0.0043 |
| Q6FIF0 | 741.8363 | 2 | −3.6 | 48.5 | 4.00E−04 |
| O95159 | 1104.0579 | 4 | 0.9 | 22.8 | 0.035 |
| P17010 | 494.7613 | 2 | 2.1 | 26.2 | 0.043 |
| P17010 | 546.9704 | 3 | 2.5 | 26.4 | 0.003 |
| Q7Z3T8 | 877.9525 | 2 | −20 | 47 | 2.30E−05 |
| Q7Z3T8 | 804.889 | 2 | 1.3 | 26.5 | 3.00E−04 |
| Q7Z3T8 | 568.3072 | 3 | 1.5 | 40.9 | 4.00E−05 |
| Q14202 | 1161.5114 | 3 | 0.59 | 28.4 | 0.0011 |
| Q5VZL5 | 485.2796 | 3 | 5.6 | 31.4 | 2.50E−04 |
| P52747 | 679.3239 | 3 | −1.5 | 59.9 | 1.20E−07 |
| P52747 | 934.1298 | 3 | −9 | 47.4 | 1.30E−06 |
| P98182 | 963.9767 | 2 | −2.9 | 23.5 | 0.0016 |
| O43296 | 589.2808 | 2 | −7.4 | 38.2 | 5.50E−05 |
| Q9NRM2 | 758.3427 | 2 | 2.5 | 25.8 | 4.70E−05 |
| Q9UL40 | 741.8506 | 4 | −9.4 | 31.6 | 5.30E−04 |
| Q9H582 | 421.2414 | 3 | 16 | 22.1 | 0.01 |
| Q9H582 | 463.9373 | 3 | 9.1 | 39.6 | 4.60E−05 |
| O15015 | 930.9646 | 2 | 1.8 | 35.8 | 4.00E−07 |
| Q6DD87 | 1035.1812 | 3 | −26 | 56 | 5.80E−09 |
| Q96JM3 | 1185.6155 | 2 | 0.71 | 35.7 | 1.20E−05 |
| Q96JM3 | 625.3273 | 4 | −12 | 44.8 | 7.50E−05 |
| P17028 | 651.0446 | 3 | 4.2 | 49.2 | 1.80E−05 |
| P17028 | 867.4384 | 2 | −8.6 | 41.9 | 0.0017 |
| P36508 | 782.9207 | 2 | 0.71 | 27.4 | 1.80E−04 |
| Q9UHR6 | 833.909 | 2 | −17 | 45.4 | 6.50E−04 |
| Q9UHR6 | 1177.5831 | 3 | −0.27 | 28.7 | 0.0058 |
| Q15942 | 738.3417 | 2 | −12 | 29.9 | 9.80E−05 |
| Q15942 | 730.359 | 2 | 8.3 | 30.9 | 0.0068 |
| Q15942 | 572.7885 | 4 | 4.1 | 43.3 | 1.40E−06 |

“E value” is the expectation value.

TABLE 3
Caspase-like cleavage sites. P4-P4′ indicates the eight amino
acid residues spanning the cleavage site, which is located between
the fourth and fifth residues of the sequence. P1 residue indicates
the residue directly preceding the cleavage site. P1′ indicates
the residue directly following the cleavage site. P1 residue # indicates
the residue number in the full-length protein sequence corresponding
to the P1 residue. Entries separated by vertical bars indicate cleavage
sites found in more than one homologous protein.

| Swiss-Prot ID | Swiss-Prot acc # | P1 residue # |
|---|---|---|
| 2A5G_HUMAN | Q13362 | 14 |
| 3MG_HUMAN | P29372 | 36 |
| 41_HUMAN | P11171 | 550 |
| 4EBP1_HUMAN | Q13541 | 25 |
| 4EBP2_HUMAN | Q13542 | 26 |
| AASD1_HUMAN | Q9BTE6 | 80 |
| ABL1_HUMAN | P00519 | 939 |
| ABLM1_HUMAN | O14639 | 567 |
| ACAP3_HUMAN | Q96P50 | 588 |
| ACINU_HUMAN | Q9UKV3 | 68 |
| ACINU_HUMAN | Q9UKV3 | 511 |
| ACINU_HUMAN | Q9UKV3 | 663 |
| ACOC_HUMAN | P21399 | 673 |
| ACSL3_HUMAN | O95573 | 571 |
| ACSL4_HUMAN | O60488 | 562 |
| ACTB_HUMAN | P60709 | 157 |
| ACTG_HUMAN | P63261 | |
| ACTN1_HUMAN | P12814 | 5 |
| ACTN1_HUMAN | P12814 | 22 |
| ACTN2_HUMAN | P35609 | 29 |
| ACTN3_HUMAN | Q08043 | 36 |
| ACTN4_HUMAN | O43707 | 41 |
| ADDA_HUMAN | P35611 | 633 |
| AEBP2_HUMAN | Q6ZN18 | 233 |
| AEDO_HUMAN | Q96SZ5 | 34 |
| AF1L2_HUMAN | Q8N4X5 | 312 |
| AF1L2_HUMAN | Q8N4X5 | 630 |
| AFTIN_HUMAN | Q6ULP2 | 339 |
| AGGF1_HUMAN | Q8N302 | 148 |
| AHNK_HUMAN | Q09666 | 575 |
| AHNK_HUMAN | Q09666 | 737 |
| AHNK_HUMAN | Q09666 | 739 |
| AHNK_HUMAN | Q09666 | 865 |
| AHNK_HUMAN | Q09666 | 919 |
| AHNK_HUMAN | Q09666 | 1168 |
| AHNK_HUMAN | Q09666 | 1424 |
| AHNK_HUMAN | Q09666 | 1583 |
| AHNK_HUMAN | Q09666 | 2711 |
| AHNK_HUMAN | Q09666 | 2882 |
| AHNK_HUMAN | Q09666 | 3464 |
| AHNK_HUMAN | Q09666 | 3493 |
| AHNK_HUMAN | Q09666 | 3718 |
| AHNK_HUMAN | Q09666 | 4358 |
| AHNK_HUMAN | Q09666 | 5580 |
| AHSA1_HUMAN | O95433 | 18 |
| AHSA1_HUMAN | O95433 | 254 |
| AHTF1_HUMAN | Q8WYP5 | 1367 |
| AIM1_HUMAN | Q9Y4K1 | 67 |
| AKA12_HUMAN | Q02952 | 451 |
| AKAP2_HUMAN | Q9Y2D5 | 472 |
| AKAP9_HUMAN | Q99996 | 1033 |
| AKNA_HUMAN | Q7Z591 | 799 |
| AKP13_HUMAN | Q12802 | 544 |
| AKP13_HUMAN | Q12802 | 829 |
| AKP13_HUMAN | Q12802 | 905 |
| AKP13_HUMAN | Q12802 | 1055 |
| AKP13_HUMAN | Q12802 | 1539 |
| AKP8L_HUMAN | Q9ULX6 | 108 |
| ALMS1_HUMAN | Q8TCU4 | 427 |
| ALMS1_HUMAN | Q8TCU4 | 590 |
| ALMS1_HUMAN | Q8TCU4 | 779 |
| ALO17_HUMAN | Q9HCF4 | 273 |
| AMPD3_HUMAN | Q01432 | 36 |
| AMPM1_HUMAN | P53582 | 12 |
| ANKH1_HUMAN | Q8IWZ3 | 4 |
| ANKH1_HUMAN | Q8IWZ3 | 1048 |
| ANKS6_HUMAN | Q68DC2 | 275 |
| ANS1A_HUMAN | Q92625 | 529 |
| ANXA2_HUMAN | P07355 | 16 |
| AXA2L_HUMAN | A6NMY6 | |
| AP1G1_HUMAN | O43747 | 689 |
| AP1G2_HUMAN | O75843 | 631 |
| AP2A2_HUMAN | O94973 | 690 |
| AP3B2_HUMAN | Q13367 | 843 |
| APBB2_HUMAN | Q92870 | 279 |
| APC_HUMAN | P25054 | 1498 |
| APMAP_HUMAN | Q9HDC9 | 22 |
| APTX_HUMAN | Q7Z2E3 | 141 |
| AR13B_HUMAN | Q3SXY8 | 241 |
| ARBK1_HUMAN | P25098 | 527 |
| ARBK1_HUMAN | P25098 | 481 |
| ARBK2_HUMAN | P35626 | |
| ARHG1_HUMAN | Q92888 | 292 |
| ARHG2_HUMAN | Q92974 | 626 |
| ARHGA_HUMAN | O15013 | 1246 |
| ARI1A_HUMAN | O14497 | 75 |
| ARI1A_HUMAN | O14497 | 606 |
| ARI4A_HUMAN | P29374 | 1030 |
| ARI4B_HUMAN | Q4LE39 | 1072 |
| ARID2_HUMAN | Q68CP9 | 625 |
| ARID2_HUMAN | Q68CP9 | 629 |
| ARM10_HUMAN | Q8N2F6 | 86 |
| ARMC6_HUMAN | Q6NXE6 | 82 |
| ARNT_HUMAN | P27540 | 151 |
| ARP2_HUMAN | P61160 | 161 |
| ARP21_HUMAN | Q9UBL0 | 494 |
| ARP3_HUMAN | P61158 | 59 |
| ARPC5_HUMAN | O15511 | 29 |
| ARPC5_HUMAN | O15511 | 32 |
| ARS2_HUMAN | Q9BXP5 | 161 |
| ASB13_HUMAN | Q8WXK3 | 51 |
| ASCC1_HUMAN | Q8N9N2 | 34 |
| ASCC2_HUMAN | Q9H1I8 | 621 |
| ASHWN_HUMAN | Q9BVC5 | 105 |
| ASPP2_HUMAN | Q13625 | 527 |
| ATAD5_HUMAN | Q96QE3 | 284 |
| ATD2B_HUMAN | Q9ULI0 | 77 |
| ATF1_HUMAN | P18846 | 46 |
| ATF4_HUMAN | P18848 | 65 |
| ATF7_HUMAN | P17544 | 43 |
| ATG3_HUMAN | Q9NT62 | 104 |
| ATRX_HUMAN | P46100 | 919 |
| ATX1L_HUMAN | P0C7T5 | 308 |
| ATX2_HUMAN | Q99700 | 842 |
| ATX2L_HUMAN | Q8WWM7 | 584 |
| ATX3_HUMAN | P54252 | 217 |
| AZI1_HUMAN | Q9UPN4 | 548 |
| BA2D1_HUMAN | Q9Y520 | 888 |
| BA2D1_HUMAN | Q9Y520 | 2189 |
| BAP1_HUMAN | Q92560 | 311 |
| BAP31_HUMAN | P51572 | 164 |
| BASP_HUMAN | P80723 | 165 |
| BASP_HUMAN | P80723 | 171 |
| BAT3_HUMAN | P46379 | 1001 |
| BAZ1A_HUMAN | Q9NRL2 | 499 |
| BCAP_HUMAN | Q6ZUJ8 | 148 |
| BCLF1_HUMAN | Q9NYF8 | 324 |
| BCLF1_HUMAN | Q9NYF8 | 382 |
| BCR_HUMAN | P11274 | 243 |
| BDP1_HUMAN | A6H8Y1 | 525 |
| BID_HUMAN | P55957 | 75 |
| BIG3_HUMAN | Q5TH69 | 292 |
| BIN1_HUMAN | O00499 | 301 |
| BIRC6_HUMAN | Q9NR09 | 461 |
| BL1S3_HUMAN | Q6QNY0 | 64 |
| BLNK_HUMAN | Q8WV28 | 177 |
| BNIP2_HUMAN | Q12982 | 83 |
| BPTF_HUMAN | Q12830 | 1625 |
| BRD1_HUMAN | O95696 | 921 |
| BRD4_HUMAN | O60885 | 337 |
| BRD8_HUMAN | Q9H0E9 | 560 |
| BTB14_HUMAN | Q96RE7 | 174 |
| BUB1_HUMAN | O43683 | 395 |
| BUD13_HUMAN | Q9BRD0 | 273 |
| C170L_HUMAN | Q96L14 | 50 |
| C1QBP_HUMAN | Q07021 | 185 |
| C2C2L_HUMAN | O14523 | 442 |
| C2D1A_HUMAN | Q6P1N0 | 30 |
| C2D1B_HUMAN | Q5T0F9 | 460 |
| CA059_HUMAN | Q5T8I9 | 13 |
| CA103_HUMAN | Q5T3J3 | 515 |
| CA163_HUMAN | Q96BR5 | 120 |
| CA165_HUMAN | Q7L4P6 | 103 |
| CA170_HUMAN | Q5SV97 | 42 |
| CA175_HUMAN | Q68CQ1 | 411 |
| CA1L1_HUMAN | Q08AD1 | 421 |
| CABL2_HUMAN | Q9BTV7 | 58 |
| CACO1_HUMAN | Q9P1Z2 | 134 |
| CADH2_HUMAN | P19022 | 799 |
| CAF1A_HUMAN | Q13111 | 110 |
| CAF1A_HUMAN | Q13111 | 614 |
| CALR_HUMAN | P27797 | 121 |
| CALR_HUMAN | P27797 | 258 |
| CALR_HUMAN | P27797 | 328 |
| CAMKV_HUMAN | Q8NCB2 | 407 |
| CAMLG_HUMAN | P49069 | 9 |
| CAMLG_HUMAN | P49069 | 115 |
| CAMP1_HUMAN | Q5T5Y3 | 751 |
| CAMP1_HUMAN | Q5T5Y3 | 1254 |
| CAPR1_HUMAN | Q14444 | 94 |
| CAPZB_HUMAN | P47756 | 149 |
| CASC3_HUMAN | O15234 | 389 |
| CASC5_HUMAN | Q8NG31 | 1194 |
| CASP_HUMAN | Q13948 | 387 |
| CUX1_HUMAN | P39880 | 376 |
| CASP3_HUMAN | P42574 | 28 |
| CASP3_HUMAN | P42574 | 175 |
| CASP7_HUMAN | P55210 | 198 |
| CATB_HUMAN | P07858 | 77 |
| CB044_HUMAN | Q9H6R7 | 508 |
| CBL_HUMAN | P22681 | 806 |
| CBWD1_HUMAN | Q9BRT8 | 184 |
| CBWD2_HUMAN | Q8IUF1 | 184 |
| CBWD3_HUMAN | Q5JTY5 | 184 |
| CBWD5_HUMAN | Q5RIA9 | 184 |
| CBWD6_HUMAN | Q4V339 | 184 |
| CBWD7_HUMAN | A6NM15 | 36 |
| CC104_HUMAN | Q96G28 | 141 |
| CC104_HUMAN | Q96G28 | 144 |
| CC124_HUMAN | Q96CT7 | 149 |
| CC131_HUMAN | O60293 | 335 |
| CC50A_HUMAN | Q9NV96 | 12 |
| CCD43_HUMAN | Q96MW1 | 16 |
| CCD53_HUMAN | Q9Y3C0 | 4 |
| CCD91_HUMAN | Q7Z6B0 | 99 |
| CCD97_HUMAN | Q96F63 | 52 |
| CCDC9_HUMAN | Q9Y3X0 | 299 |
| CCNT2_HUMAN | O60583 | 454 |
| CD2L1_HUMAN | P21127 | 405 |
| CD2L5_HUMAN | Q14004 | 1353 |
| CDC27_HUMAN | P30260 | 236 |
| CDC27_HUMAN | P30260 | 243 |
| CDC5L_HUMAN | Q99459 | 391 |
| CDCA7_HUMAN | Q9BWT1 | 39 |
| CDV3_HUMAN | Q9UKY7 | 122 |
| CDYL1_HUMAN | Q9Y232 | 210 |
| CE022_HUMAN | Q49AR2 | 196 |
| CE152_HUMAN | O94986 | 62 |
| CE170_HUMAN | Q5SW79 | 936 |
| CE170_HUMAN | Q5SW79 | 1324 |
| CEBPZ_HUMAN | Q03701 | 774 |
| CEBPZ_HUMAN | Q03701 | 917 |
| CEBPZ_HUMAN | Q03701 | 955 |
| CH041_HUMAN | Q6NXR4 | 4 |
| CH082_HUMAN | Q6P1X6 | 25 |
| CH60_HUMAN | P10809 | 111 |
| CH60_HUMAN | P10809 | 452 |
| CH60_HUMAN | P10809 | 504 |
| CHD3_HUMAN | Q12873 | 372 |
| CHD4_HUMAN | Q14839 | 363 |
| CHD5_HUMAN | Q8TDI0 | 336 |
| CHD4_HUMAN | Q14839 | 1233 |
| CHD7_HUMAN | Q9P2D1 | 2285 |
| CHM4A_HUMAN | Q9BY43 | 80 |
| CHM4B_HUMAN | Q9H444 | 83 |
| CHM4C_HUMAN | Q96CF2 | 83 |
| CI080_HUMAN | Q9NRY2 | 57 |
| CJ018_HUMAN | Q5VWN6 | 1207 |
| CJ047_HUMAN | Q86WR7 | 109 |
| CK059_HUMAN | Q6IAA8 | 72 |
| CL035_HUMAN | Q9HCM1 | 359 |
| CL035_HUMAN | Q9HCM1 | 501 |
| CL043_HUMAN | Q96C57 | 72 |
| CL043_HUMAN | Q96C57 | 204 |
| CLAP1_HUMAN | Q7Z460 | 1218 |
| CLCA_HUMAN | P09496 | 76 |
| CLCA_HUMAN | P09496 | 92 |
| CLIC1_HUMAN | O00299 | 141 |
| CLIP1_HUMAN | P30622 | 397 |
| CLSPN_HUMAN | Q9HAW4 | 563 |
| CND2_HUMAN | Q15003 | 170 |
| CND2_HUMAN | Q15003 | 199 |
| CND2_HUMAN | Q15003 | 366 |
| CND2_HUMAN | Q15003 | 380 |
| CNDH2_HUMAN | Q6IBW4 | 459 |
| CO6A3_HUMAN | P12111 | 2615 |
| COBL1_HUMAN | Q53SF7 | 983 |
| COPA_HUMAN | P53621 | 188 |
| COPA_HUMAN | P53621 | 856 |
| COPB2_HUMAN | P35606 | 854 |
| COR1A_HUMAN | P31146 | 394 |
| CP088_HUMAN | Q1ED39 | 182 |
| CP110_HUMAN | Q7Z7A1 | 801 |
| CP110_HUMAN | Q7Z7A1 | 1395 |
| CPIN1_HUMAN | Q6FI81 | 214 |
| CPNE1_HUMAN | Q99829 | 464 |
| CPNE3_HUMAN | O75131 | 428 |
| CPSF6_HUMAN | Q16630 | 54 |
| CPSF7_HUMAN | Q8N684 | 29 |
| CPSF7_HUMAN | Q8N684 | 33 |
| CPSF7_HUMAN | Q8N684 | 324 |
| CPZIP_HUMAN | Q6JBY9 | 272 |
| CQ056_HUMAN | Q96N21 | 380 |
| CQ085_HUMAN | Q53F19 | 157 |
| CQ085_HUMAN | Q53F19 | 231 |
| CR025_HUMAN | Q96B23 | 44 |
| CREB1_HUMAN | P16220 | 116 |
| CREB1_HUMAN | P16220 | 229 |
| CROCC_HUMAN | Q5TZA2 | 578 |
| CS043_HUMAN | Q9BQ61 | 62 |
| CS044_HUMAN | Q9H6X5 | 368 |
| CSN1_HUMAN | Q13098 | 94 |
| CSRN2_HUMAN | Q9H175 | 39 |
| CSTF3_HUMAN | Q12996 | 576 |
| CTBL1_HUMAN | Q8WYA6 | 66 |
| CTCF_HUMAN | P49711 | 46 |
| CTNB1_HUMAN | P35222 | 115 |
| CTND1_HUMAN | O60716 | 161 |
| CTR9_HUMAN | Q6PD62 | 1120 |
| CUL4B_HUMAN | Q13620 | 25 |
| CUTC_HUMAN | Q9NTM9 | 33 |
| CUX1_HUMAN | P39880 | 1339 |
| CYB5B_HUMAN | O43169 | 10 |
| DBPA_HUMAN | P16989 | 144 |
| DBPA_HUMAN | P16989 | 161 |
| DBPA_HUMAN | P16989 | 269 |
| DBPA_HUMAN | P16989 | 137 |
| YBOX1_HUMAN | P67809 | 105 |
| YBOX2_HUMAN | Q9Y2T7 | 140 |
| DCNL2_HUMAN | Q6PH85 | 42 |
| DCTN1_HUMAN | Q14203 | 302 |
| DD19A_HUMAN | Q9NUU7 | 4 |
| DDX1_HUMAN | Q92499 | 439 |
| DDX24_HUMAN | Q9GZR7 | 296 |
| DDX46_HUMAN | Q7L014 | 871 |
| DDX46_HUMAN | Q7L014 | 922 |
| DDX59_HUMAN | Q5T1V6 | 43 |
| DESM_HUMAN | P17661 | 264 |
| DFFA_HUMAN | O00273 | 6 |
| DFFA_HUMAN | O00273 | 221 |
| DGCR8_HUMAN | Q8WYQ5 | 248 |
| DGCR8_HUMAN | Q8WYQ5 | 396 |
| DGKH_HUMAN | Q86XP1 | 582 |
| DGKH_HUMAN | Q86XP1 | 698 |
| DGLB_HUMAN | Q8NCG7 | 548 |
| DHAK_HUMAN | Q3LXA3 | 362 |
| DHX30_HUMAN | Q7L2E3 | 206 |
| DHX37_HUMAN | Q8IY37 | 573 |
| DHX9_HUMAN | Q08211 | 96 |
| DHX9_HUMAN | Q08211 | 167 |
| DIAP1_HUMAN | O60610 | 648 |
| DIDO1_HUMAN | Q9BTC0 | 987 |
| DIDO1_HUMAN | Q9BTC0 | 1250 |
| DIDO1_HUMAN | Q9BTC0 | 1352 |
| DIDO1_HUMAN | Q9BTC0 | 1518 |
| DLG1_HUMAN | Q12959 | 412 |
| DNJC7_HUMAN | Q99615 | 8 |
| DNM1L_HUMAN | O00429 | 503 |
| DNM1L_HUMAN | O00429 | 579 |
| DNM3A_HUMAN | Q9Y6K1 | 438 |
| DOC10_HUMAN | Q96BY6 | 327 |
| DOHH_HUMAN | Q9BU89 | 8 |
| DOT1L_HUMAN | Q8TEK3 | 1333 |
| DP13A_HUMAN | Q9UKG1 | 444 |
| DPOD1_HUMAN | P28340 | 102 |
| DPOLA_HUMAN | P09884 | 83 |
| DPP9_HUMAN | Q86TI2 | 13 |
| DPYL4_HUMAN | O14531 | 456 |
| DREB_HUMAN | Q16643 | 340 |
| DREB_HUMAN | Q16643 | 477 |
| DSRAD_HUMAN | P55265 | 214 |
| DTL_HUMAN | Q9NZJ0 | 578 |
| DTX3L_HUMAN | Q8TDB6 | 217 |
| DYHC1_HUMAN | Q14204 | 4220 |
| DYHC1_HUMAN | Q14204 | 4367 |
| E400N_HUMAN | Q6ZTU2 | 183 |
| EP400_HUMAN | Q96L91 | 194 |
| E41L2_HUMAN | O43491 | 912 |
| EAP1_HUMAN | Q9H1B7 | 132 |
| EBP2_HUMAN | Q99848 | 211 |
| ECE1_HUMAN | P42892 | 33 |
| ECT2_HUMAN | Q9H8V3 | 628 |
| EDC4_HUMAN | Q6P2E9 | 57 |
| EDC4_HUMAN | Q6P2E9 | 485 |
| EDC4_HUMAN | Q6P2E9 | 490 |
| EDC4_HUMAN | Q6P2E9 | 662 |
| EDC4_HUMAN | Q6P2E9 | 796 |
| EDRF1_HUMAN | Q3B7T1 | 115 |
| EEA1_HUMAN | Q15075 | 127 |
| EEA1_HUMAN | Q15075 | 132 |
| EF1A1_HUMAN | P68104 | 403 |
| EF1A3_HUMAN | Q5VTE0 | |
| EF1B_HUMAN | P24534 | 102 |
| EF1D_HUMAN | P29692 | 158 |
| EF2_HUMAN | P13639 | 611 |
| EH1L1_HUMAN | Q8N3D4 | 1329 |
| EHBP1_HUMAN | Q8NDI1 | 274 |
| EHD1_HUMAN | Q9H4M9 | 415 |
| EHMT1_HUMAN | Q9H9B1 | 329 |
| EHMT1_HUMAN | Q9H9B1 | 481 |
| EHMT2_HUMAN | Q96KQ7 | 453 |
| EIF3B_HUMAN | P55884 | 184 |
| EIF3G_HUMAN | O75821 | 7 |
| EIF3J_HUMAN | O75822 | 50 |
| ELF1_HUMAN | P32519 | 145 |
| ENOA_HUMAN | P06733 | 203 |
| ENPL_HUMAN | P14625 | 28 |
| ENPL_HUMAN | P14625 | 59 |
| EP15_HUMAN | P42566 | 618 |
| EP15R_HUMAN | Q9UBC2 | 569 |
| EPC1_HUMAN | Q9H2F5 | 27 |
| EPN1_HUMAN | Q9Y6I3 | 460 |
| EPN2_HUMAN | O95208 | 339 |
| ERC6L_HUMAN | Q2NKX8 | 801 |
| ERCC6_HUMAN | Q03468 | 52 |
| ERF_HUMAN | P50548 | 191 |
| ERF3A_HUMAN | P15170 | 39 |
| ERIC1_HUMAN | Q86X53 | 276 |
| ESYT2_HUMAN | A0FGR8 | 759 |
| ETUD1_HUMAN | Q7Z2Z2 | 932 |
| EXDL2_HUMAN | Q9NVH0 | 198 |
| F101B_HUMAN | Q8N5W9 | 61 |
| F107B_HUMAN | Q9H098 | 5 |
| F117B_HUMAN | Q6P1L5 | 374 |
| F125A_HUMAN | Q96EY5 | 172 |
| F169A_HUMAN | Q9Y6X4 | 446 |
| FA13A_HUMAN | O94988 | 594 |
| FA21A_HUMAN | Q641Q2 | 1134 |
| FA21B_HUMAN | Q5SNT6 | 1046 |
| FA21C_HUMAN | Q9Y4E1 | 1113 |
| FA21D_HUMAN | Q5SRD0 | 101 |
| FA29A_HUMAN | Q7Z4H7 | 568 |
| FA44A_HUMAN | Q8NFC6 | 1483 |
| FA44A_HUMAN | Q8NFC6 | 1708 |
| FA44A_HUMAN | Q8NFC6 | 2044 |
| FAS_HUMAN | P49327 | 1165 |
| FETUA_HUMAN | P02765 | 133 |
| FIP1_HUMAN | Q6UN15 | 158 |
| FKB15_HUMAN | Q5T1M5 | 306 |
| FLI1_HUMAN | Q01543 | 20 |
| FLNA_HUMAN | P21333 | 25 |
| FLNA_HUMAN | P21333 | 1048 |
| FLNA_HUMAN | P21333 | 1336 |
| FLNA_HUMAN | P21333 | 1504 |
| FLNA_HUMAN | P21333 | 2536 |
| FLNA_HUMAN | P21333 | 34 |
| FLNB_HUMAN | O75369 | 7 |
| FLNC_HUMAN | Q14315 | 27 |
| FLNB_HUMAN | O75369 | 478 |
| FLNB_HUMAN | O75369 | 1021 |
| FLNB_HUMAN | O75369 | 1476 |
| FNBP1_HUMAN | Q96RU3 | 519 |
| FNBP4_HUMAN | Q8N3X1 | 153 |
| FNBP4_HUMAN | Q8N3X1 | 425 |
| FNBP4_HUMAN | Q8N3X1 | 777 |
| FOXJ2_HUMAN | Q9P0K8 | 212 |
| FOXK1_HUMAN | P85037 | 80 |
| FOXO3_HUMAN | O43524 | 54 |
| FOXP4_HUMAN | Q8IVH2 | 406 |
| FRAP_HUMAN | P42345 | 2459 |
| FRYL_HUMAN | O94915 | 1512 |
| FUBP1_HUMAN | Q96AE4 | 83 |
| FUBP1_HUMAN | Q96AE4 | 181 |
| FUBP1_HUMAN | Q96AE4 | 139 |
| FUBP2_HUMAN | Q92945 | 183 |
| FUBP2_HUMAN | Q92945 | 128 |
| FUBP3_HUMAN | Q96I24 | 34 |
| FUBP3_HUMAN | Q96I24 | 159 |
| FUS_HUMAN | P35637 | 355 |
| FXR2_HUMAN | P51116 | 561 |
| FYB_HUMAN | O15117 | 446 |
| FYB_HUMAN | O15117 | 655 |
| FYN_HUMAN | P06241 | 19 |
| FYTD1_HUMAN | Q96QD9 | 31 |
| THOC4_HUMAN | Q86V81 | 6 |
| FYV1_HUMAN | Q9Y2I7 | 989 |
| FYV1_HUMAN | Q9Y2I7 | 1607 |
| G3P_HUMAN | P04406 | 89 |
| GABP1_HUMAN | Q06547 | 303 |
| GABP2_HUMAN | Q8TAK5 | 304 |
| GALT_HUMAN | P07902 | 18 |
| GAPD1_HUMAN | Q14C86 | 1102 |
| GATA2_HUMAN | P23769 | 46 |
| GBF1_HUMAN | Q92538 | 368 |
| GCFC_HUMAN | Q9Y5B6 | 221 |
| GCP2_HUMAN | Q9BSJ2 | 772 |
| GCP60_HUMAN | Q9H3P7 | 15 |
| GCP60_HUMAN | Q9H3P7 | 343 |
| GDIR2_HUMAN | P52566 | 19 |
| GDIR2_HUMAN | P52566 | 55 |
| GELS_HUMAN | P06396 | 403 |
| GELS_HUMAN | P06396 | 639 |
| GEMI5_HUMAN | Q8TEQ6 | 1319 |
| GEMI8_HUMAN | Q9NWZ8 | 169 |
| GEN_HUMAN | Q17RS7 | 623 |
| GFPT1_HUMAN | Q06210 | 260 |
| GGA3_HUMAN | Q9NZ52 | 333 |
| GGA3_HUMAN | Q9NZ52 | 517 |
| GIT1_HUMAN | Q9Y2X7 | 418 |
| GIT1_HUMAN | Q9Y2X7 | 632 |
| GIT2_HUMAN | Q14161 | 625 |
| GLGB_HUMAN | Q04446 | 307 |
| GLRX3_HUMAN | O76003 | 101 |
| GLU2B_HUMAN | P14314 | 94 |
| GLU2B_HUMAN | P14314 | 101 |
| GLU2B_HUMAN | P14314 | 226 |
| GMIP_HUMAN | Q9P107 | 424 |
| GMIP_HUMAN | Q9P107 | 472 |
| GMIP_HUMAN | Q9P107 | 842 |
| GNL1_HUMAN | P36915 | 49 |
| GNL1_HUMAN | P36915 | 52 |
| GNL1_HUMAN | P36915 | 343 |
| GOGB1_HUMAN | Q14789 | 1245 |
| GOGB1_HUMAN | Q14789 | 1801 |
| GON4L_HUMAN | Q3T8J9 | 481 |
| GPKOW_HUMAN | Q92917 | 37 |
| GPKOW_HUMAN | Q92917 | 98 |
| GPN1_HUMAN | Q9HCN4 | 311 |
| GPTC8_HUMAN | Q9UKJ3 | 882 |
| GRDN_HUMAN | Q3V6T2 | 219 |
| GRDN_HUMAN | Q3V6T2 | 484 |
| GRIN1_HUMAN | Q7Z2K8 | 306 |
| GSDMD_HUMAN | P57764 | 87 |
| GSDMD_HUMAN | P57764 | 275 |
| GSTP1_HUMAN | P09211 | 91 |
| GTF2I_HUMAN | P78347 | 105 |
| H2AY_HUMAN | O75367 | 172 |
| H4_HUMAN | P62805 | 25 |
| H4_HUMAN | P62805 | 69 |
| HAP28_HUMAN | Q13442 | 24 |
| HBS1L_HUMAN | Q9Y450 | 29 |
| HCLS1_HUMAN | P14317 | 26 |
| HDAC4_HUMAN | P56524 | 8 |
| HDAC4_HUMAN | P56524 | 289 |
| HDAC6_HUMAN | Q9UBN7 | 1088 |
| HDAC7_HUMAN | Q8WUI4 | 412 |
| HDC_HUMAN | Q9UBI9 | 323 |
| HDGR2_HUMAN | Q7Z4V5 | 30 |
| HDGR2_HUMAN | Q7Z4V5 | 241 |
| HECD1_HUMAN | Q9ULT8 | 1492 |
| HELLS_HUMAN | Q9NRZ9 | 22 |
| HG2A_HUMAN | P04233 | 22 |
| HIRP3_HUMAN | Q9BW71 | 110 |
| HJURP_HUMAN | Q8NCD3 | 91 |
| HMHA1_HUMAN | Q92619 | 39 |
| HMHA1_HUMAN | Q92619 | 262 |
| HMHA1_HUMAN | Q92619 | 662 |
| HMOX2_HUMAN | P30519 | 251 |
| HNRH1_HUMAN | P31943 | 340 |
| HNRH2_HUMAN | P55795 | 340 |
| HNRH3_HUMAN | P31942 | 144 |
| HNRL1_HUMAN | Q9BUJ2 | 96 |
| HNRL2_HUMAN | Q1KMD3 | 126 |
| HNRLL_HUMAN | Q8WVV9 | 289 |
| HNRPD_HUMAN | Q14103 | 69 |
| HNRPF_HUMAN | P52597 | 251 |
| HNRPG_HUMAN | P38159 | 233 |
| HNRPG_HUMAN | P38159 | 283 |
| HNRPK_HUMAN | P61978 | 128 |
| HNRPK_HUMAN | P61978 | 346 |
| HNRPK_HUMAN | P61978 | 370 |
| HNRPL_HUMAN | P14866 | 284 |
| HNRPQ_HUMAN | O60506 | 468 |
| HOOK1_HUMAN | Q9UJC3 | 233 |
| HOOK2_HUMAN | Q96ED9 | 160 |
| HPS4_HUMAN | Q9NQG7 | 495 |
| HRX_HUMAN | Q03164 | 2384 |
| HRX_HUMAN | Q03164 | 2718 |
| HS105_HUMAN | Q92598 | 547 |
| HSP74_HUMAN | P34932 | 727 |
| HSP7C_HUMAN | P11142 | 80 |
| HTF4_HUMAN | Q99081 | 22 |
| HTSF1_HUMAN | O43719 | 33 |
| HTSF1_HUMAN | O43719 | 39 |
| HTSF1_HUMAN | O43719 | 80 |
| HUWE1_HUMAN | Q7Z6Z7 | 2017 |
| HUWE1_HUMAN | Q7Z6Z7 | 2359 |
| HUWE1_HUMAN | Q7Z6Z7 | 2473 |
| HUWE1_HUMAN | Q7Z6Z7 | 2930 |
| HUWE1_HUMAN | Q7Z6Z7 | 3079 |
| HUWE1_HUMAN | Q7Z6Z7 | 3664 |
| I2BP2_HUMAN | Q7Z5L9 | 495 |
| I5P2_HUMAN | P32019 | 263 |
| IASPP_HUMAN | Q8WUF5 | 294 |
| ICAL_HUMAN | P20810 | 233 |
| ICAL_HUMAN | P20810 | 348 |
| ICAL_HUMAN | P20810 | 513 |
| ICAL_HUMAN | P20810 | 659 |
| IF2BL_HUMAN | A6NK07 | 118 |
| IF2B_HUMAN | P20042 | |
| IF2P_HUMAN | O60841 | 20 |
| IF4A2_HUMAN | Q14240 | 21 |
| IF4B_HUMAN | P23588 | 50 |
| IF4B_HUMAN | P23588 | 59 |
| IF4G1_HUMAN | Q04637 | 414 |
| IF4G1_HUMAN | Q04637 | 532 |
| IF4G1_HUMAN | Q04637 | 665 |
| IF4G2_HUMAN | P78344 | 792 |
| IF4G3_HUMAN | O43432 | 478 |
| IF4H_HUMAN | Q15056 | 93 |
| IF5A1_HUMAN | P63241 | 96 |
| IF5A1_HUMAN | P63241 | 6 |
| IF5AL_HUMAN | Q6IS14 | |
| IF5A2_HUMAN | Q9GZV4 | 6 |
| IKBB_HUMAN | Q15653 | 159 |
| IKBL2_HUMAN | Q96HA7 | 498 |
| IKZF1_HUMAN | Q13422 | 367 |
| IKZF2_HUMAN | Q9UKS7 | 7 |
| IKZF5_HUMAN | Q9H5V7 | 225 |
| ILF3_HUMAN | Q12906 | 287 |
| ILF3_HUMAN | Q12906 | 439 |
| ILKAP_HUMAN | Q9H0C8 | 39 |
| IMA1_HUMAN | P52294 | 64 |
| IMA7_HUMAN | O60684 | 69 |
| IMDH2_HUMAN | P12268 | 172 |
| IN80D_HUMAN | Q53TQ3 | 678 |
| INF2_HUMAN | Q27J81 | 1051 |
| INF2_HUMAN | Q27J81 | 1146 |
| IPO9_HUMAN | Q96P70 | 963 |
| IQEC1_HUMAN | Q6DN90 | 234 |
| IQGA1_HUMAN | P46940 | 8 |
| IRF2_HUMAN | P14316 | 237 |
| IRS4_HUMAN | O14654 | 716 |
| ISY1_HUMAN | Q9ULR0 | 167 |
| IWS1_HUMAN | Q96ST2 | 347 |
| JHD3C_HUMAN | Q9H3R0 | 396 |
| JIP4_HUMAN | O60271 | 5 |
| JIP4_HUMAN | O60271 | 213 |
| JIP4_HUMAN | O60271 | 284 |
| JKIP1_HUMAN | Q96N16 | 17 |
| JMY_HUMAN | Q8N9B5 | 722 |
| JOSD3_HUMAN | Q9H5J8 | 10 |
| JSPR1_HUMAN | Q96MG2 | 12 |
| K0174_HUMAN | P53990 | 197 |
| K0232_HUMAN | Q92628 | 556 |
| K0515_HUMAN | Q5JSZ5 | 1082 |
| K0515_HUMAN | Q5JSZ5 | 1235 |
| K0831_HUMAN | Q6ZNE5 | 28 |
| K0831_HUMAN | Q6ZNE5 | 226 |
| K1462_HUMAN | Q9P266 | 1179 |
| K1543_HUMAN | Q9P1Y5 | 861 |
| K1627_HUMAN | Q9HCE5 | 29 |
| K1704_HUMAN | Q8IXQ4 | 88 |
| K1967_HUMAN | Q8N163 | 292 |
| K1967_HUMAN | Q8N163 | 618 |
| K1967_HUMAN | Q8N163 | 768 |
| KHDR1_HUMAN | Q07666 | 75 |
| KI67_HUMAN | P46013 | 173 |
| KI67_HUMAN | P46013 | 410 |
| KI67_HUMAN | P46013 | 2147 |
| KIF15_HUMAN | Q9NS87 | 1133 |
| KKCC1_HUMAN | Q8N5S9 | 32 |
| KLF12_HUMAN | Q9Y4X4 | 73 |
| KPYM_HUMAN | P14618 | 354 |
| KRI1_HUMAN | Q8N9T8 | 312 |
| KRR1_HUMAN | Q13601 | 38 |
| KS6A4_HUMAN | O75676 | 377 |
| KU86_HUMAN | P13010 | 455 |
| KU86_HUMAN | P13010 | 556 |
| LAGE3_HUMAN | Q14657 | 28 |
| LAMB1_HUMAN | P07942 | 1358 |
| LAP2A_HUMAN | P42166 | 441 |
| LAP2A_HUMAN | P42166 | 486 |
| LAP4_HUMAN | Q14160 | 501 |
| LAP4_HUMAN | Q14160 | 635 |
| LAP4_HUMAN | Q14160 | 1197 |
| LARP1_HUMAN | Q6PKG0 | 172 |
| LARP1_HUMAN | Q6PKG0 | 495 |
| LARP4_HUMAN | Q71RC2 | 573 |
| LARP5_HUMAN | Q92615 | 135 |
| LAT_HUMAN | O43561 | 167 |
| LCAP_HUMAN | Q9UIQ6 | 29 |
| LCORL_HUMAN | Q8N3X6 | 229 |
| LCOR_HUMAN | Q96JN0 | 80 |
| LIMA1_HUMAN | Q9UHB6 | 345 |
| LIN37_HUMAN | Q96GY3 | 23 |
| LIN7C_HUMAN | Q9NUP9 | 62 |
| LIPA1_HUMAN | Q13136 | 218 |
| LIPB2_HUMAN | Q8ND30 | 31 |
| LMNB1_HUMAN | P20700 | 146 |
| LMO7_HUMAN | Q8WWI1 | 962 |
| LMTK2_HUMAN | Q8IWU2 | 900 |
| LNP_HUMAN | Q9C0E8 | 368 |
| LPP_HUMAN | Q93052 | 403 |
| LRBA_HUMAN | P50851 | 1756 |
| LRBA_HUMAN | P50851 | 1784 |
| LRC47_HUMAN | Q8N1G4 | 525 |
| LRCH1_HUMAN | Q9Y2L9 | 405 |
| LRCH2_HUMAN | Q5VUJ6 | 603 |
| LRCH3_HUMAN | Q96II8 | 642 |
| LRCH4_HUMAN | O75427 | 358 |
| LRMP_HUMAN | Q12912 | 181 |
| LRRF1_HUMAN | Q32MZ4 | 415 |
| LRRF2_HUMAN | Q9Y608 | 531 |
| LSM11_HUMAN | P83369 | 305 |
| LSM3_HUMAN | P62310 | 6 |
| LSP1_HUMAN | P33241 | 102 |
| LTV1_HUMAN | Q96GA3 | 205 |
| LYRIC_HUMAN | Q86UE4 | 183 |
| M6PBP_HUMAN | O60664 | 9 |
| M6PBP_HUMAN | O60664 | 219 |
| M6PBP_HUMAN | O60664 | 222 |
| MA7D1_HUMAN | Q3KQU3 | 570 |
| MACF1_HUMAN | Q9UPN3 | 1523 |
| MACF1_HUMAN | Q9UPN3 | 1726 |
| MACF4_HUMAN | Q96PK2 | 2228 |
| MACF1_HUMAN | Q9UPN3 | 3020 |
| MACF4_HUMAN | Q96PK2 | 3522 |
| MADD_HUMAN | Q8WXG6 | 1177 |
| MAGD1_HUMAN | Q9Y5V3 | 222 |
| MAGG1_HUMAN | Q96MG7 | 41 |
| MAOM_HUMAN | P23368 | 379 |
| MAP1A_HUMAN | P78559 | 1884 |
| MAP4_HUMAN | P27816 | 8 |
| MAP4_HUMAN | P27816 | 46 |
| MAP4_HUMAN | P27816 | 151 |
| MAP4_HUMAN | P27816 | 249 |
| MAP4_HUMAN | P27816 | 327 |
| MAP9_HUMAN | Q49MG5 | 119 |
| MARE1_HUMAN | Q15691 | 116 |
| MARK1_HUMAN | Q9P0L2 | 22 |
| MATR3_HUMAN | P43243 | 187 |
| MATR3_HUMAN | P43243 | 452 |
| MATR3_HUMAN | P43243 | 680 |
| MATR3_HUMAN | P43243 | 703 |
| MATR3_HUMAN | P43243 | 763 |
| MAVS_HUMAN | Q7Z434 | 490 |
| MAX_HUMAN | P61244 | 48 |
| MBB1A_HUMAN | Q9BQG0 | 749 |
| MCM2_HUMAN | P49736 | 68 |
| MCM2_HUMAN | P49736 | 88 |
| MCM3_HUMAN | P25205 | 703 |
| MCM4_HUMAN | P33991 | 132 |
| MCM5_HUMAN | P33992 | 13 |
| MCM6_HUMAN | Q14566 | 274 |
| MDC1_HUMAN | Q14676 | 1035 |
| MDN1_HUMAN | Q9NU22 | 5127 |
| MED1_HUMAN | Q15648 | 930 |
| MED1_HUMAN | Q15648 | 1484 |
| MED14_HUMAN | O60244 | 994 |
| MED26_HUMAN | O95402 | 407 |
| MEF2C_HUMAN | Q06413 | 105 |
| METK2_HUMAN | P31153 | 39 |
| MEX3B_HUMAN | Q6ZN04 | 354 |
| MGAP_HUMAN | Q8IWI9 | 339 |
| MGAP_HUMAN | Q8IWI9 | 571 |
| MGAP_HUMAN | Q8IWI9 | 680 |
| MIA3_HUMAN | Q5JRA6 | 709 |
| MIER1_HUMAN | Q8N108 | 51 |
| MINT_HUMAN | Q96T58 | 1574 |
| MINT_HUMAN | Q96T58 | 2007 |
| MINT_HUMAN | Q96T58 | 2859 |
| MISSL_HUMAN | Q8NDC0 | 9 |
| MKL1_HUMAN | Q969V6 | 121 |
| MKL2_HUMAN | Q9ULH7 | 182 |
| MLL2_HUMAN | O14686 | 386 |
| MLL2_HUMAN | O14686 | 1865 |
| MLL3_HUMAN | Q8NEZ4 | 2188 |
| MOBL3_HUMAN | Q9Y3A3 | 34 |
| MOES_HUMAN | P26038 | 114 |
| MORC3_HUMAN | Q14149 | 664 |
| MORC3_HUMAN | Q14149 | 751 |
| MOT1_HUMAN | P53985 | 469 |
| MP2K1_HUMAN | Q02750 | 16 |
| MP2K1_HUMAN | Q02750 | 282 |
| MPP10_HUMAN | O00566 | 545 |
| MPP8_HUMAN | Q99549 | 19 |
| MPP8_HUMAN | Q99549 | 501 |
| MPP8_HUMAN | Q99549 | 516 |
| MRP_HUMAN | P49006 | 63 |
| MSPD2_HUMAN | Q8NHP6 | 274 |
| MTA70_HUMAN | Q86U44 | 334 |
| MYH10_HUMAN | P35580 | 1160 |
| MYH10_HUMAN | P35580 | 1309 |
| MYH11_HUMAN | P35749 | 1160 |
| MYH9_HUMAN | P35579 | 1153 |
| MYH9_HUMAN | P35579 | 1375 |
| MYO9B_HUMAN | Q13459 | 1703 |
| MYPT1_HUMAN | O14974 | 885 |
| N4BP1_HUMAN | O75113 | 490 |
| NACA_HUMAN | Q13765 | 42 |
| NADAP_HUMAN | Q9BWU0 | 537 |
| NAG_HUMAN | A2RRP1 | 636 |
| NAIF1_HUMAN | Q69YI7 | 102 |
| NARF_HUMAN | Q9UHQ1 | 272 |
| NARF_HUMAN | Q9UHQ1 | 291 |
| NASP_HUMAN | P49321 | 19 |
| NASP_HUMAN | P49321 | 32 |
| NCK1_HUMAN | P16333 | 88 |
| NCOA3_HUMAN | Q9Y6Q9 | 1012 |
| NCOA5_HUMAN | Q9HCD5 | 153 |
| NCOA5_HUMAN | Q9HCD5 | 380 |
| NCOA6_HUMAN | Q14686 | 1461 |
| NCOR1_HUMAN | O75376 | 385 |
| NCOR1_HUMAN | O75376 | 555 |
| NCOR1_HUMAN | O75376 | 1826 |
| NCOR2_HUMAN | Q9Y618 | 377 |
| NCOR2_HUMAN | Q9Y618 | 1926 |
| NDRG1_HUMAN | Q92597 | 9 |
| NEB2_HUMAN | Q96SB3 | 551 |
| NED4L_HUMAN | Q96PU5 | 345 |
| NEDD1_HUMAN | Q8NHV4 | 434 |
| NEDD4_HUMAN | P46934 | 279 |
| NEK1_HUMAN | Q96PY6 | 949 |
| NEK4_HUMAN | P51957 | 380 |
| NEK9_HUMAN | Q8TD19 | 841 |
| NELFA_HUMAN | Q9H3P2 | 299 |
| NFAC1_HUMAN | O95644 | 110 |
| NFAC2_HUMAN | Q13469 | 66 |
| NFKB2_HUMAN | Q00653 | 10 |
| NFRKB_HUMAN | Q6P4R8 | 5 |
| NFRKB_HUMAN | Q6P4R8 | 496 |
| NHERF_HUMAN | O14745 | 4 |
| NIPA_HUMAN | Q86WB0 | 295 |
| NIPA_HUMAN | Q86WB0 | 449 |
| NIPBL_HUMAN | Q6KC79 | 472 |
| NKTR_HUMAN | P30414 | 959 |
| NOL1_HUMAN | P46087 | 207 |
| NOL1_HUMAN | P46087 | 230 |
| NOL5_HUMAN | Q9Y2X3 | 124 |
| NOP14_HUMAN | P78316 | 319 |
| NP1L1_HUMAN | P55209 | 57 |
| NP1L1_HUMAN | P55209 | 183 |
| NP1L4_HUMAN | Q99733 | 8 |
| NP1L4_HUMAN | Q99733 | 46 |
| NP60_HUMAN | Q49A26 | 255 |
| NPAT_HUMAN | Q14207 | 733 |
| NPM_HUMAN | P06748 | 6 |
| NS1BP_HUMAN | Q9Y6Y0 | 238 |
| NSBP1_HUMAN | P82970 | 57 |
| NSUN2_HUMAN | Q08J23 | 108 |
| NSUN2_HUMAN | Q08J23 | 499 |
| NSUN2_HUMAN | Q08J23 | 664 |
| NU153_HUMAN | P49790 | 358 |
| NUCB2_HUMAN | P80303 | 237 |
| NUCB2_HUMAN | P80303 | 258 |
| NUCKS_HUMAN | Q9H1E3 | 29 |
| NUCL_HUMAN | P19338 | 636 |
| NUDC3_HUMAN | Q8IVD9 | 119 |
| NUDC3_HUMAN | Q8IVD9 | 125 |
| NUFP2_HUMAN | Q7Z417 | 451 |
| NUMA1_HUMAN | Q14980 | 1747 |
| NUMA1_HUMAN | Q14980 | 1829 |
| NUP43_HUMAN | Q8NFH3 | 58 |
| NUP50_HUMAN | Q9UKX7 | 126 |
| NUP93_HUMAN | Q8N1F7 | 157 |
| ODPB_HUMAN | P11177 | 37 |
| OFD1_HUMAN | O75665 | 853 |
| ORAV1_HUMAN | Q8WV07 | 9 |
| OSBL8_HUMAN | Q9BZF1 | 806 |
| OTU6B_HUMAN | Q8N6M0 | 80 |
| OTUD4_HUMAN | Q01804 | 9 |
| OXR1_HUMAN | Q8N573 | 449 |
| P4R3A_HUMAN | Q6IN85 | 692 |
| P66B_HUMAN | Q8WXI9 | 344 |
| PA24A_HUMAN | P47712 | 522 |
| PABP2_HUMAN | Q86U42 | 111 |
| PAIRB_HUMAN | Q8NC51 | 337 |
| PAK1_HUMAN | Q13153 | 90 |
| PAK2_HUMAN | Q13177 | 89 |
| PAK2_HUMAN | Q13177 | 148 |
| PALLD_HUMAN | Q8WX93 | 432 |
| PARG_HUMAN | Q86W56 | 256 |
| PARP1_HUMAN | P09874 | 72 |
| PARP1_HUMAN | P09874 | 214 |
| PAWR_HUMAN | Q96IZ0 | 131 |
| PAXI_HUMAN | P49023 | 5 |
| PAXI_HUMAN | P49023 | 102 |
| PAXI_HUMAN | P49023 | 335 |
| PB1_HUMAN | Q86U86 | 21 |
| PCBP1_HUMAN | Q15365 | 203 |
| PCBP1_HUMAN | Q15365 | 220 |
| PCBP1_HUMAN | Q15365 | 275 |
| PCBP2_HUMAN | Q15366 | 282 |
| PCF11_HUMAN | O94913 | 1288 |
| PCM1_HUMAN | Q15154 | 193 |
| PCM1_HUMAN | Q15154 | 1551 |
| PCNT_HUMAN | O95613 | 80 |
| PDIP3_HUMAN | Q9BY77 | 234 |
| PDLI1_HUMAN | O00151 | 54 |
| PDXD1_HUMAN | Q6P996 | 584 |
| PEBB_HUMAN | Q13951 | 120 |
| PFTK1_HUMAN | O94921 | 56 |
| PGK1_HUMAN | P00558 | 68 |
| PGK1_HUMAN | P00558 | 98 |
| PGK1_HUMAN | P00558 | 285 |
| PGK1_HUMAN | P00558 | 159 |
| PGK2_HUMAN | P07205 | |
| PHAR4_HUMAN | Q8IZ21 | 20 |
| PHF3_HUMAN | Q92576 | 1099 |
| PHF3_HUMAN | Q92576 | 1157 |
| PHF3_HUMAN | Q92576 | 1397 |
| PHF3_HUMAN | Q92576 | 1626 |
| PHTNS_HUMAN | Q6NYC8 | 495 |
| PI4KB_HUMAN | Q9UBF8 | 488 |
| PIAS1_HUMAN | O75925 | 100 |
| PICAL_HUMAN | Q13492 | 276 |
| PITM1_HUMAN | O00562 | 378 |
| PJA2_HUMAN | O43164 | 86 |
| PKHG1_HUMAN | Q9ULL1 | 435 |
| PKP4_HUMAN | Q99569 | 803 |
| PLCG1_HUMAN | P19174 | 770 |
| PLDN_HUMAN | Q9UL45 | 10 |
| POGZ_HUMAN | Q7Z3K3 | 27 |
| POMP_HUMAN | Q9Y244 | 12 |
| PP1RA_HUMAN | Q96QC0 | 293 |
| PP1RA_HUMAN | Q96QC0 | 366 |
| PP1RA_HUMAN | Q96QC0 | 376 |
| PP4R1_HUMAN | Q8TF05 | 444 |
| PPIA_HUMAN | P62937 | 9 |
| PPIL4_HUMAN | Q8WUA2 | 232 |
| PPR3D_HUMAN | O95685 | 31 |
| PR40A_HUMAN | O75400 | 133 |
| PRD15_HUMAN | P57071 | 1269 |
| PRKDC_HUMAN | P78527 | 3211 |
| PROF1_HUMAN | P07737 | 14 |
| PROF1_HUMAN | P07737 | 19 |
| PROF1_HUMAN | P07737 | 81 |
| PRP17_HUMAN | O60508 | 55 |
| PRP17_HUMAN | O60508 | 190 |
| PRP17_HUMAN | O60508 | 204 |
| PRP31_HUMAN | Q8WWY3 | 386 |
| PRR12_HUMAN | Q9ULL5 | 115 |
| PRR3_HUMAN | P79522 | 31 |
| PRS10_HUMAN | P62333 | 265 |
| PRS6A_HUMAN | P17980 | 27 |
| PRS6A_HUMAN | P17980 | 318 |
| PRS6B_HUMAN | P43686 | 297 |
| PRS8_HUMAN | P62195 | 252 |
| PSA5_HUMAN | P28066 | 71 |
| PSA7L_HUMAN | Q8TAA3 | 15 |
| PSA7_HUMAN | O14818 | 13 |
| PSB1_HUMAN | P20618 | 47 |
| PSB4_HUMAN | P28070 | 29 |
| PSB7_HUMAN | Q99436 | 53 |
| PSD12_HUMAN | O00232 | 19 |
| PSD4_HUMAN | Q8NDX1 | 82 |
| PSD4_HUMAN | Q8NDX1 | 535 |
| PSIP1_HUMAN | O75475 | 30 |
| PSIP1_HUMAN | O75475 | 433 |
| PSME3_HUMAN | P61289 | 77 |
| PTBP1_HUMAN | P26599 | 139 |
| PTBP1_HUMAN | P26599 | 172 |
| PTCA_HUMAN | Q14761 | 116 |
| PTCA_HUMAN | Q14761 | 120 |
| PTMA_HUMAN | P06454 | 7 |
| PTN3_HUMAN | P26045 | 471 |
| PUR2_HUMAN | P22102 | 205 |
| PUR2_HUMAN | P22102 | 225 |
| PUR2_HUMAN | P22102 | 443 |
| PUR6_HUMAN | P22234 | 26 |
| PUR6_HUMAN | P22234 | 319 |
| PUR9_HUMAN | P31939 | 339 |
| PUS7_HUMAN | Q96PZ0 | 22 |
| PUS7_HUMAN | Q96PZ0 | 50 |
| PWP2A_HUMAN | Q96N64 | 55 |
| PYR1_HUMAN | P27708 | 1138 |
| QKI_HUMAN | Q96PU8 | 74 |
| QN1_HUMAN | Q5TB80 | 247 |
| QSER1_HUMAN | Q2KHR3 | 1321 |
| QSK_HUMAN | Q9Y2K2 | 383 |
| R3HD1_HUMAN | Q15032 | 499 |
| RA1L3_HUMAN | P0C7M2 | 69 |
| ROA1_HUMAN | P09651 | |
| RA1L3_HUMAN | P0C7M2 | 157 |
| ROA1_HUMAN | P09651 | |
| RAD21_HUMAN | O60216 | 128 |
| RAD21_HUMAN | O60216 | 279 |
| RADIL_HUMAN | Q96JH8 | 841 |
| RANG_HUMAN | P43487 | 127 |
| RB_HUMAN | P06400 | 346 |
| RB3GP_HUMAN | Q15042 | 252 |
| RBBP4_HUMAN | Q09028 | 361 |
| RBBP7_HUMAN | Q16576 | 360 |
| RBBP6_HUMAN | Q7Z6E9 | 972 |
| RBBP6_HUMAN | Q7Z6E9 | 1267 |
| RBBP6_HUMAN | Q7Z6E9 | 1678 |
| RBBP7_HUMAN | Q16576 | 93 |
| RBBP7_HUMAN | Q16576 | 98 |
| RBBP8_HUMAN | Q99708 | 742 |
| RBM15_HUMAN | Q96T37 | 750 |
| RBM16_HUMAN | Q9UPN6 | 380 |
| RBM16_HUMAN | Q9UPN6 | 775 |
| RBM25_HUMAN | P49756 | 633 |
| RBM26_HUMAN | Q5T8P6 | 280 |
| RBM26_HUMAN | Q5T8P6 | 431 |
| RBM27_HUMAN | Q9P2N5 | 487 |
| RBM28_HUMAN | Q9NW13 | 244 |
| RBM33_HUMAN | Q96EV2 | 998 |
| RBM39_HUMAN | Q14498 | 331 |
| RBM8A_HUMAN | Q9Y5S9 | 6 |
| RBM8A_HUMAN | Q9Y5S9 | 55 |
| RBM9_HUMAN | O43251 | 102 |
| RBP2_HUMAN | P49792 | 1157 |
| RBP2_HUMAN | P49792 | 2490 |
| RBP2_HUMAN | P49792 | 2860 |
| RBP2_HUMAN | P49792 | 3131 |
| RBP2_HUMAN | P49792 | 2306 |
| RGPD1_HUMAN | Q68DN6 | 1315 |
| RGPD3_HUMAN | A6NKT7 | 1331 |
| RGPD4_HUMAN | Q7Z3J3 | 1331 |
| RGPD5_HUMAN | Q99666 | 1330 |
| RGPD6_HUMAN | Q53T03 | 1330 |
| RGPD8_HUMAN | O14715 | 320 |
| RBP2_HUMAN | P49792 | 2236 |
| RGPD3_HUMAN | A6NKT7 | 1261 |
| RGPD4_HUMAN | Q7Z3J3 | 1261 |
| RGPD5_HUMAN | Q99666 | 1260 |
| RGPD6_HUMAN | Q53T03 | 1260 |
| RGPD8_HUMAN | O14715 | 250 |
| RBP56_HUMAN | Q92804 | 140 |
| RBTN1_HUMAN | P25800 | 8 |
| RBY1B_HUMAN | A6NDE4 | 466 |
| RBY1F_HUMAN | Q15415 | 466 |
| RBY1H_HUMAN | Q15378 | 326 |
| RCC2_HUMAN | Q9P258 | 60 |
| RCN2_HUMAN | Q14257 | 203 |
| RCOR2_HUMAN | Q8IZ40 | 391 |
| RD23B_HUMAN | P54727 | 165 |
| RED_HUMAN | Q13123 | 108 |
| RED_HUMAN | Q13123 | 324 |
| REL_HUMAN | Q04864 | 86 |
| RENT1_HUMAN | Q92900 | 75 |
| REPS1_HUMAN | Q96D71 | 386 |
| REPS1_HUMAN | Q96D71 | 459 |
| REPS1_HUMAN | Q96D71 | 465 |
| REQU_HUMAN | Q92785 | 115 |
| REQU_HUMAN | Q92785 | 243 |
| REST_HUMAN | Q13127 | 941 |
| RFC1_HUMAN | P35251 | 167 |
| RFC1_HUMAN | P35251 | 723 |
| RFX7_HUMAN | Q2KHR2 | 479 |
| RGAP1_HUMAN | Q9H0H5 | 273 |
| RGPD1_HUMAN | Q68DN6 | 1499 |
| RGPD2_HUMAN | P0C839 | 764 |
| RGPD3_HUMAN | A6NKT7 | 1515 |
| RGPD4_HUMAN | Q7Z3J3 | 1515 |
| RGPD5_HUMAN | Q99666 | 1514 |
| RGPD6_HUMAN | Q53T03 | 1514 |
| RGPD8_HUMAN | O14715 | 504 |
| RGS10_HUMAN | O43665 | 12 |
| RGS10_HUMAN | O43665 | 14 |
| RHG04_HUMAN | P98171 | 403 |
| RHG25_HUMAN | P42331 | 387 |
| RHG25_HUMAN | P42331 | 397 |
| RHG30_HUMAN | Q7Z6I6 | 363 |
| RHG30_HUMAN | Q7Z6I6 | 592 |
| RHG30_HUMAN | Q7Z6I6 | 907 |
| RHGBA_HUMAN | Q6P4F7 | 256 |
| RHOA_HUMAN | P61586 | 90 |
| RHOC_HUMAN | P08134 | |
| RIF1_HUMAN | Q5UIP0 | 1809 |
| RIF1_HUMAN | Q5UIP0 | 2000 |
| RIMB1_HUMAN | O95153 | 44 |
| RIMB1_HUMAN | O95153 | 1807 |
| RING1_HUMAN | Q06587 | 31 |
| RIOK1_HUMAN | Q9BRS2 | 129 |
| RIPK1_HUMAN | Q13546 | 558 |
| RIR2_HUMAN | P31350 | 29 |
| RL17_HUMAN | P18621 | 110 |
| RL5_HUMAN | P46777 | 136 |
| RL5_HUMAN | P46777 | 168 |
| RN168_HUMAN | Q8IYW5 | 250 |
| RN213_HUMAN | Q63HN8 | 355 |
| RN219_HUMAN | Q5W0B1 | 433 |
| RN220_HUMAN | Q5VTB9 | 413 |
| RNF5_HUMAN | Q99942 | 8 |
| RNZ1_HUMAN | Q9H777 | 279 |
| ROA0_HUMAN | Q13151 | 62 |
| ROA0_HUMAN | Q13151 | 73 |
| ROA2_HUMAN | P22626 | 76 |
| ROA2_HUMAN | P22626 | 130 |
| ROA3_HUMAN | P51991 | 90 |
| ROA3_HUMAN | P51991 | 115 |
| ROA3_HUMAN | P51991 | 178 |
| ROCK1_HUMAN | Q13464 | 1113 |
| RPAP3_HUMAN | Q9H6T3 | 124 |
| RPAP3_HUMAN | Q9H6T3 | 451 |
| RPB9_HUMAN | P36954 | 4 |
| RPC4_HUMAN | P05423 | 131 |
| RPC5_HUMAN | Q9NVU0 | 543 |
| RPGF6_HUMAN | Q8TEU7 | 1282 |
| RREB1_HUMAN | Q92766 | 1173 |
| RRMJ3_HUMAN | Q8IY81 | 346 |
| RRP12_HUMAN | Q5JTH9 | 556 |
| RRP12_HUMAN | Q5JTH9 | 1161 |
| RRP1B_HUMAN | Q14684 | 275 |
| RS20_HUMAN | P60866 | 5 |
| RS23_HUMAN | P62266 | 88 |
| RS28_HUMAN | P62857 | 54 |
| RS3_HUMAN | P23396 | 32 |
| RSRC1_HUMAN | Q96IZ7 | 238 |
| RTF1_HUMAN | Q92541 | 140 |
| RTN4_HUMAN | Q9NQC3 | 84 |
| RTN4_HUMAN | Q9NQC3 | 905 |
| RU1C_HUMAN | P09234 | 10 |
| RU2A_HUMAN | P09661 | 45 |
| RUSD2_HUMAN | Q8IZ73 | 441 |
| RUXF_HUMAN | P62306 | 52 |
| S11IP_HUMAN | Q8N1F8 | 372 |
| S12A2_HUMAN | P55011 | 66 |
| S2546_HUMAN | Q96AG3 | 10 |
| S30BP_HUMAN | Q9UHR5 | 44 |
| SAFB1_HUMAN | Q15424 | 146 |
| SAFB1_HUMAN | Q15424 | 262 |
| SAFB2_HUMAN | Q14151 | 261 |
| SAFB1_HUMAN | Q15424 | 360 |
| SAFB2_HUMAN | Q14151 | 359 |
| SAFB1_HUMAN | Q15424 | 796 |
| SAFB2_HUMAN | Q14151 | 820 |
| SAFB2_HUMAN | Q14151 | 153 |
| SAFB2_HUMAN | Q14151 | 183 |
| SAHH2_HUMAN | O43865 | 5 |
| SAHH2_HUMAN | O43865 | 73 |
| SAHH2_HUMAN | O43865 | 83 |
| SAHH3_HUMAN | Q96HN2 | 109 |
| SAM4B_HUMAN | Q5PRF9 | 412 |
| SAP_HUMAN | P07602 | 312 |
| SAP_HUMAN | P07602 | 405 |
| SAPS1_HUMAN | Q9UPN7 | 358 |
| SASH3_HUMAN | O75995 | 55 |
| SASH3_HUMAN | O75995 | 115 |
| SATB1_HUMAN | Q01826 | 254 |
| SATT_HUMAN | P43007 | 12 |
| SC16A_HUMAN | O15027 | 341 |
| SC16A_HUMAN | O15027 | 837 |
| SC24B_HUMAN | O95487 | 295 |
| SCAM3_HUMAN | O14828 | 39 |
| SCMH1_HUMAN | Q96GD3 | 511 |
| SCO1_HUMAN | O75880 | 188 |
| SCOC_HUMAN | Q9UIL1 | 87 |
| SDCG1_HUMAN | O60524 | 779 |
| SEC13_HUMAN | P55735 | 14 |
| SEC20_HUMAN | Q12981 | 32 |
| SENP6_HUMAN | Q9GZR1 | 49 |
| SEPT9_HUMAN | Q9UHD8 | 282 |
| SETD2_HUMAN | Q9BYW2 | 647 |
| SETD2_HUMAN | Q9BYW2 | 1169 |
| SETX_HUMAN | Q7Z333 | 1534 |
| SF01_HUMAN | Q15637 | 448 |
| SF3A1_HUMAN | Q15459 | 32 |
| SF3A1_HUMAN | Q15459 | 503 |
| SF3B1_HUMAN | O75533 | 34 |
| SF3B2_HUMAN | Q13435 | 291 |
| SF3B2_HUMAN | Q13435 | 753 |
| SF3B4_HUMAN | Q15427 | 12 |
| SFPQ_HUMAN | P23246 | 525 |
| SFR14_HUMAN | Q8IX01 | 732 |
| SFR14_HUMAN | Q8IX01 | 901 |
| SFR14_HUMAN | Q8IX01 | 922 |
| SFRIP_HUMAN | Q99590 | 407 |
| SFRS2_HUMAN | Q01130 | 70 |
| SFRS2_HUMAN | Q01130 | 73 |
| SFRS3_HUMAN | P84103 | 4 |
| SFRS5_HUMAN | Q13243 | 52 |
| SFRS6_HUMAN | Q13247 | 167 |
| SGOL1_HUMAN | Q5FBB7 | 206 |
| SH2D3_HUMAN | Q8N5H7 | 375 |
| SHOT1_HUMAN | A0MZ66 | 129 |
| SIPA1_HUMAN | Q96FS4 | 814 |
| SIX4_HUMAN | Q9UIU6 | 296 |
| SKI_HUMAN | P12755 | 527 |
| SKT_HUMAN | Q5T5P2 | 609 |
| SLD5_HUMAN | Q9BRT9 | 6 |
| SLK_HUMAN | Q9H2G2 | 403 |
| SLMAP_HUMAN | Q14BN4 | 464 |
| SLU7_HUMAN | O95391 | 7 |
| SMC2_HUMAN | O95347 | 1116 |
| SMCA4_HUMAN | P51532 | 1381 |
| SMCE1_HUMAN | Q969G3 | 264 |
| SMHD1_HUMAN | A6NHR9 | 5 |
| SMRC2_HUMAN | Q8TAQ2 | 814 |
| SMRD2_HUMAN | Q92925 | 135 |
| SNPC4_HUMAN | Q5SXM2 | 1168 |
| SNX12_HUMAN | Q9UMY4 | 21 |
| SNX2_HUMAN | O60749 | 84 |
| SNX29_HUMAN | Q8TEQ0 | 182 |
| SNX3_HUMAN | O60493 | 32 |
| SNX6_HUMAN | Q9UNH7 | 10 |
| SOBP_HUMAN | A7XYQ1 | 298 |
| SODC_HUMAN | P00441 | 93 |
| SODC_HUMAN | P00441 | 102 |
| SON_HUMAN | P18583 | 153 |
| SON_HUMAN | P18583 | 352 |
| SON_HUMAN | P18583 | 1640 |
| SON_HUMAN | P18583 | 1718 |
| SP1_HUMAN | P08047 | 199 |
| SP110_HUMAN | Q9HB58 | 353 |
| SP3_HUMAN | Q02447 | 275 |
| SP3_HUMAN | Q02447 | 530 |
| SPAS2_HUMAN | Q86XZ4 | 145 |
| SPAST_HUMAN | Q9UBP0 | 470 |
| SPD2B_HUMAN | A1X283 | 682 |
| SPEC1_HUMAN | Q5M775 | 213 |
| SPEE_HUMAN | P19623 | 6 |
| SPF27_HUMAN | O75934 | 14 |
| SPF30_HUMAN | O75940 | 62 |
| SPG20_HUMAN | Q8N0X7 | 496 |
| SPS2L_HUMAN | Q9NUQ6 | 119 |
| SPT6H_HUMAN | Q7KZ85 | 1047 |
| SPTA2_HUMAN | Q13813 | 500 |
| SPTA2_HUMAN | Q13813 | 1478 |
| SPTN2_HUMAN | O15020 | 1752 |
| SR140_HUMAN | O15042 | 704 |
| SR140_HUMAN | O15042 | 712 |
| SR140_HUMAN | O15042 | 725 |
| SR140_HUMAN | O15042 | 737 |
| SRC_HUMAN | P12931 | 45 |
| SRCAP_HUMAN | Q6ZRS2 | 2275 |
| SRFB1_HUMAN | Q8NEF9 | 211 |
| SRP68_HUMAN | Q9UHB9 | 537 |
| SRPK1_HUMAN | Q96SB4 | 412 |
| SRRM2_HUMAN | Q9UQ35 | 147 |
| SRRM2_HUMAN | Q9UQ35 | 1149 |
| SSA27_HUMAN | O60232 | 81 |
| SSBP3_HUMAN | Q9BWW4 | 286 |
| SSF1_HUMAN | Q9NQ55 | 245 |
| SSFA2_HUMAN | P28290 | 627 |
| SSH2_HUMAN | Q76I76 | 963 |
| SSRP1_HUMAN | Q08945 | 173 |
| STAP1_HUMAN | Q9ULZ2 | 170 |
| STK10_HUMAN | O94804 | 332 |
| STK24_HUMAN | Q9Y6E0 | 325 |
| STK39_HUMAN | Q9UEW8 | 435 |
| STK4_HUMAN | Q13043 | 349 |
| STRN_HUMAN | O43815 | 35 |
| STRN_HUMAN | O43815 | 436 |
| STX10_HUMAN | O60499 | 138 |
| STX10_HUMAN | O60499 | 196 |
| STX12_HUMAN | Q86Y82 | 217 |
| STX17_HUMAN | P56962 | 201 |
| STX7_HUMAN | O15400 | 204 |
| SUGT1_HUMAN | Q9Y2Z0 | 20 |
| SYAP1_HUMAN | Q96A49 | 281 |
| SYEP_HUMAN | P07814 | 929 |
| SYF2_HUMAN | O95926 | 12 |
| SYG_HUMAN | P41250 | 56 |
| SYMPK_HUMAN | Q92797 | 28 |
| SYNC_HUMAN | O43776 | 409 |
| SYNE1_HUMAN | Q8NF91 | 8279 |
| SYNE2_HUMAN | Q8WXH0 | 4215 |
| SYWC_HUMAN | P23381 | 83 |
| T106B_HUMAN | Q9NUM4 | 19 |
| T106C_HUMAN | Q9BVX2 | 23 |
| T2EA_HUMAN | P29083 | 303 |
| T2FA_HUMAN | P35269 | 272 |
| TACC1_HUMAN | O75410 | 323 |
| TACC1_HUMAN | O75410 | 500 |
| TACC2_HUMAN | O95359 | 371 |
| TACC3_HUMAN | Q9Y6A5 | 21 |
| TACC3_HUMAN | Q9Y6A5 | 286 |
| TAD1L_HUMAN | Q96BN2 | 78 |
| TAF11_HUMAN | Q15544 | 34 |
| TAF7_HUMAN | Q15545 | 100 |
| TBA1A_HUMAN | Q71U36 | 33 |
| TBA1B_HUMAN | P68363 | |
| TBA1C_HUMAN | Q9BQE3 | |
| TBA3C_HUMAN | Q13748 | |
| TBA3E_HUMAN | Q6PEY2 | |
| TBA1A_HUMAN | Q71U36 | 245 |
| TBA1B_HUMAN | P68363 | |
| TBA1C_HUMAN | Q9BQE3 | |
| TBA3C_HUMAN | Q13748 | |
| TBA3E_HUMAN | Q6PEY2 | |
| TBA4A_HUMAN | P68366 | |
| TBA8_HUMAN | Q9NY65 | |
| TBB2A_HUMAN | Q13885 | 114 |
| TBB2B_HUMAN | Q9BVA1 | |
| TBB2C_HUMAN | P68371 | |
| TBB3_HUMAN | Q13509 | |
| TBB5_HUMAN | P07437 | |
| TBB2C_HUMAN | P68371 | 114 |
| TBB5_HUMAN | P07437 | |
| TBCC_HUMAN | Q15814 | 153 |
| TBCD4_HUMAN | O60343 | 272 |
| TBCD4_HUMAN | O60343 | 275 |
| TBL1R_HUMAN | Q9BZK7 | 152 |
| TBL1R_HUMAN | Q9BZK7 | 85 |
| TBL1X_HUMAN | O60907 | |
| TBL1R_HUMAN | Q9BZK7 | 152 |
| TBL1Y_HUMAN | Q9BQ87 | 162 |
| TBL1X_HUMAN | O60907 | 164 |
| TCEA1_HUMAN | P23193 | 124 |
| TCF20_HUMAN | Q9UGU0 | 1219 |
| TCOF_HUMAN | Q13428 | 1101 |
| TCOF_HUMAN | Q13428 | 1242 |
| TCPD_HUMAN | P50991 | 268 |
| TCPD_HUMAN | P50991 | 456 |
| TCPE_HUMAN | P48643 | 65 |
| TCPE_HUMAN | P48643 | 153 |
| TCPZ_HUMAN | P40227 | 404 |
| TCTP_HUMAN | P13693 | 25 |
| TDRD6_HUMAN | O60522 | 1918 |
| TEX2_HUMAN | Q8IWB9 | 96 |
| TEX2_HUMAN | Q8IWB9 | 356 |
| TF2B_HUMAN | Q00403 | 207 |
| TF2L1_HUMAN | Q9NZI6 | 22 |
| TFCP2_HUMAN | Q12800 | 42 |
| UBIP1_HUMAN | Q9NZI7 | 39 |
| TF3A_HUMAN | Q92664 | 18 |
| TF65_HUMAN | Q04206 | 97 |
| TGS1_HUMAN | Q96RS0 | 337 |
| TGS1_HUMAN | Q96RS0 | 343 |
| THOC4_HUMAN | Q86V81 | 93 |
| THOC5_HUMAN | Q13769 | 17 |
| THOP1_HUMAN | P52888 | 13 |
| TIF1A_HUMAN | O15164 | 784 |
| TIF1B_HUMAN | Q13263 | 105 |
| TIF1B_HUMAN | Q13263 | 148 |
| TIF1B_HUMAN | Q13263 | 685 |
| TIF1B_HUMAN | Q13263 | 688 |
| TIF1B_HUMAN | Q13263 | 726 |
| TIM_HUMAN | Q9UNS1 | 579 |
| TINF2_HUMAN | Q9BSI4 | 207 |
| TLK2_HUMAN | Q86UE8 | 132 |
| TM168_HUMAN | Q9H0V1 | 426 |
| TM1L2_HUMAN | Q6ZVM7 | 157 |
| TMUB1_HUMAN | Q9BVT8 | 60 |
| TNIP2_HUMAN | Q8NFZ5 | 194 |
| TNR6A_HUMAN | Q8NDV7 | 1542 |
| TOE1_HUMAN | Q96GM8 | 7 |
| TOE1_HUMAN | Q96GM8 | 373 |
| TOIP1_HUMAN | Q5JTV8 | 226 |
| TOIP1_HUMAN | Q5JTV8 | 304 |
| TOLIP_HUMAN | Q9H0E2 | 36 |
| TOM1_HUMAN | O60784 | 157 |
| TOM1_HUMAN | O60784 | 179 |
| TOM1_HUMAN | O60784 | 184 |
| TOM1_HUMAN | O60784 | 393 |
| TOP2B_HUMAN | Q02880 | 1470 |
| TP53B_HUMAN | Q12888 | 211 |
| TP53B_HUMAN | Q12888 | 317 |
| TP53B_HUMAN | Q12888 | 829 |
| TP53B_HUMAN | Q12888 | 1478 |
| TPR_HUMAN | P12270 | 1837 |
| TPR_HUMAN | P12270 | 2147 |
| TPRGL_HUMAN | Q5T0D9 | 9 |
| TR150_HUMAN | Q9Y2W1 | 574 |
| TRBP2_HUMAN | Q15633 | 234 |
| TREF1_HUMAN | Q96PN7 | 760 |
| TRI33_HUMAN | Q9UPN9 | 829 |
| TRIP4_HUMAN | Q15650 | 122 |
| TRIP4_HUMAN | Q15650 | 288 |
| TRM1L_HUMAN | Q7Z2T5 | 44 |
| TRS85_HUMAN | Q9Y2L5 | 853 |
| TSC1_HUMAN | Q92574 | 638 |
| TSR1_HUMAN | Q2NL82 | 332 |
| TTC1_HUMAN | Q99614 | 65 |
| TTC4_HUMAN | O95801 | 254 |
| TTF2_HUMAN | Q9UNY4 | 826 |
| TYB10_HUMAN | P63313 | 6 |
| TYSY_HUMAN | P04818 | 119 |
| TYY1_HUMAN | P25490 | 119 |
| U119A_HUMAN | Q13432 | 44 |
| U119B_HUMAN | A6NIH7 | 51 |
| U2AF2_HUMAN | P26368 | 128 |
| UAP1L_HUMAN | Q3KQV9 | 299 |
| UAP56_HUMAN | Q13838 | 25 |
| UBA1_HUMAN | P22314 | 427 |
| UBA3_HUMAN | Q8TBC4 | 25 |
| UBAP2_HUMAN | Q5T6F2 | 201 |
| UBAP2_HUMAN | Q5T6F2 | 262 |
| UBAP2_HUMAN | Q5T6F2 | 854 |
| UBE2O_HUMAN | Q9C0C9 | 437 |
| UBE2O_HUMAN | Q9C0C9 | 1225 |
| UBFD1_HUMAN | O14562 | 232 |
| UBN1_HUMAN | Q9NPG3 | 136 |
| UBP10_HUMAN | Q14694 | 125 |
| UBP10_HUMAN | Q14694 | 138 |
| UBP10_HUMAN | Q14694 | 217 |
| UBP14_HUMAN | P54578 | 76 |
| UBP14_HUMAN | P54578 | 227 |
| UBP19_HUMAN | O94966 | 619 |
| UBP2L_HUMAN | Q14157 | 298 |
| UBP2L_HUMAN | Q14157 | 411 |
| UBP2L_HUMAN | Q14157 | 850 |
| UBP34_HUMAN | Q70CQ2 | 3366 |
| UBP36_HUMAN | Q9P275 | 576 |
| UBP42_HUMAN | Q9H9J4 | 764 |
| UBP5_HUMAN | P45974 | 134 |
| UBP5_HUMAN | P45974 | 767 |
| UBP5_HUMAN | P45974 | 782 |
| UBP7_HUMAN | Q93009 | 50 |
| UBQL1_HUMAN | Q9UMX0 | 15 |
| UBR4_HUMAN | Q5T4S7 | 2903 |
| UBXN7_HUMAN | O94888 | 109 |
| UBXN7_HUMAN | O94888 | 400 |
| UGPA_HUMAN | Q16851 | 15 |
| UH1BL_HUMAN | A0JNW5 | 1173 |
| UHRF1_HUMAN | Q96T88 | 118 |
| URP2_HUMAN | Q86UX7 | 344 |
| USE1_HUMAN | Q9NZ43 | 129 |
| USF2_HUMAN | Q15853 | 120 |
| USO1_HUMAN | O60763 | 757 |
| UTRO_HUMAN | P46939 | 261 |
| VAMP2_HUMAN | P63027 | 68 |
| VAMP3_HUMAN | Q15836 | 51 |
| VATD_HUMAN | Q9Y5K8 | 117 |
| VIME_HUMAN | P08670 | 82 |
| VIME_HUMAN | P08670 | 85 |
| VIME_HUMAN | P08670 | 90 |
| VIME_HUMAN | P08670 | 257 |
| VIME_HUMAN | P08670 | 259 |
| VIME_HUMAN | P08670 | 331 |
| VIME_HUMAN | P08670 | 429 |
| VP13D_HUMAN | Q5THJ4 | 2610 |
| VPS4A_HUMAN | Q9UN37 | 230 |
| VRK1_HUMAN | Q99986 | 231 |
| WAPL_HUMAN | Q7Z5K2 | 154 |
| WASF1_HUMAN | Q92558 | 247 |
| WASF2_HUMAN | Q9Y6W5 | 242 |
| WASF2_HUMAN | Q9Y6W5 | 411 |
| WASH1_HUMAN | A8K0Z3 | 298 |
| WDR33_HUMAN | Q9C0J8 | 1183 |
| WDR44_HUMAN | Q5JSH3 | 83 |
| WDR55_HUMAN | Q9H6Y2 | 20 |
| WDR62_HUMAN | O43379 | 1301 |
| WDR92_HUMAN | Q96MX6 | 118 |
| WFS1_HUMAN | O76024 | 75 |
| WFS1_HUMAN | O76024 | 211 |
| WIPF1_HUMAN | O43516 | 181 |
| WNK1_HUMAN | Q9H4A3 | 652 |
| WNK1_HUMAN | Q9H4A3 | 1069 |
| WNK1_HUMAN | Q9H4A3 | 2025 |
| WRIP1_HUMAN | Q96S55 | 192 |
| WWC2_HUMAN | Q6AWC2 | 855 |
| XPA_HUMAN | P23025 | 5 |
| YAP1_HUMAN | P46937 | 111 |
| YBOX1_HUMAN | P67809 | 24 |
| YBOX1_HUMAN | P67809 | 112 |
| YIPF3_HUMAN | Q9GZM5 | 68 |
| YJ005_HUMAN | Q6ZSR9 | 117 |
| YJ005_HUMAN | Q6ZSR9 | 123 |
| YM017_HUMAN | A8MX80 | 223 |
| YTDC2_HUMAN | Q9H6S0 | 324 |
| YTHD1_HUMAN | Q9BYJ9 | 164 |
| YTHD2_HUMAN | Q9Y5A9 | 166 |
| YTHD2_HUMAN | Q9Y5A9 | 367 |
| YTHD3_HUMAN | Q7Z739 | 168 |
| ZAP70_HUMAN | P43403 | 290 |
| ZBT34_HUMAN | Q8NCN2 | 139 |
| ZBT44_HUMAN | Q8NCP5 | 157 |
| ZC11A_HUMAN | O75152 | 348 |
| ZC11A_HUMAN | O75152 | 530 |
| ZC3H4_HUMAN | Q9UPT8 | 67 |
| ZC3H4_HUMAN | Q9UPT8 | 741 |
| ZC3HD_HUMAN | Q5T200 | 159 |
| ZC3HE_HUMAN | Q6PJT7 | 523 |
| ZCCHV_HUMAN | Q7Z2W4 | 433 |
| ZCCHV_HUMAN | Q7Z2W4 | 491 |
| ZCH18_HUMAN | Q86VM9 | 361 |
| ZCHC2_HUMAN | Q9C0B9 | 234 |
| ZCHC8_HUMAN | Q6NZY4 | 343 |
| ZEB1_HUMAN | P37275 | 49 |
| ZF161_HUMAN | O43829 | 243 |
| ZFAN6_HUMAN | Q6FIF0 | 106 |
| ZFAN6_HUMAN | Q6FIF0 | 126 |
| ZFPL1_HUMAN | O95159 | 171 |
| ZFX_HUMAN | P17010 | 244 |
| ZFY16_HUMAN | Q7Z3T8 | 107 |
| ZFY16_HUMAN | Q7Z3T8 | 283 |
| ZFY16_HUMAN | Q7Z3T8 | 534 |
| ZMYM3_HUMAN | Q14202 | 255 |
| ZMYM4_HUMAN | Q5VZL5 | 928 |
| ZN143_HUMAN | P52747 | 151 |
| ZN143_HUMAN | P52747 | 182 |
| ZN200_HUMAN | P98182 | 188 |
| ZN264_HUMAN | O43296 | 159 |
| ZN277_HUMAN | Q9NRM2 | 6 |
| ZN346_HUMAN | Q9UL40 | 13 |
| ZN644_HUMAN | Q9H582 | 615 |
| ZN646_HUMAN | O15015 | 1005 |
| ZN787_HUMAN | Q6DD87 | 230 |
| ZN828_HUMAN | Q96JM3 | 585 |
| ZNF24_HUMAN | P17028 | 9 |
| ZNF76_HUMAN | P36508 | 13 |
| ZNHI2_HUMAN | Q9UHR6 | 144 |
| ZYX_HUMAN | Q15942 | 149 |

### Example 13

### Patient Samples and N-Terminal Labeling

Additional unique markers were identified as follows. All patient samples were obtained through human subject protocols approved by the UCSF Committee on Human Research. Whole blood was centrifuged after collection and plasma (citrate or EDTA anticoagulant) was stored at −80° C. until processing for experiments. For discovery MS 1.5 mL of plasma was used; 0.25 mL or 0.5 mL was used for SRM experiments. N-terminal labeling was performed similarly to previously described (Wildes D & Wells J A (2010) Sampling the N-terminal proteome of human blood. Proc Natl Acad Sci USA 107(10):4561-4566). Isolated N-terminal peptides for discovery were fractionated using reverse phase high pH chromatography prior to MS analysis (Shimbo K, et al. (2012) Quantitative profiling of caspase-cleaved substrates reveals different drug-induced and cell-type patterns in apoptosis. Proc Natl Acad Sci USA 109(31): 12432-12437).

### Example 14

### Cell Culture Studies

MM1.S and SU-DHL-8 lines were obtained from ATCC; MOLM-13 from DMSZ. Cell lines were grown in RPMI-1640 media without fetal bovine serum for 24 h prior to drug treatment at indicated doses. After treatment, cells and debris were separated from media by low speed (800×g, 5 min), followed by high speed (24,000×g, 1 h) centrifugation. Total protein in media was precipitated with trichloroacetic acid, resuspended in 8M guanidine HCl, then subjected to N-terminal labeling as described (Wiita A P, et al. (2013) Global cellular response to chemotherapy-induced apoptosis. Elife 2:e01236).

### Example 15

### Mass Spectrometry

Unbiased discovery experiments and targeted discovery were analyzed on an AB SCIEX QSTAR Elite QqTOF instrument and a Thermo LTQ Orbitrap Velos instrument, respectively, with in-line low pH reverse phase chromatography (see Supplementary Materials and Methods for details of MS parameters). Crude synthetic peptides matching proteolytic N-terminal peptides found in discovery experiments were purchased from JPT (Germany). SRM methods were developed as described previously (see Wiita A P, et al., supra) and applied to unfractionated samples on an AB SCIEX QTRAP 5500 triple quadrupole instrument. Intensity normalization between pre- and post-chemotherapy samples was performed using spike-in protein standards.

### Example 16

### Smac ELISA

ELISA testing was typically performed at 1:2 plasma dilution in assay buffer using the manufacturer's protocol (RayBioTech).

### Example 17

### Pipeline-Based Approach to Proteolytic Biomarker Identification

It was hypothesized that tumor cells undergoing apoptosis in response to cytotoxic chemotherapy would release proteolytic peptides to the extracellular space over a time course of hours to days. In complex biological samples, the engineered enzyme subtiligase was used to biotin-tag free protein N-termini and isolate them on streptavidin-coated beads. After trypsinization and elution, liquid chromatography-MS (LC-MS) methods were used to either identify or quantify the N-terminal peptides in the sample (Wiita A P, Seaman J E, & Wells J A (Global analysis of cellular proteolysis by selective enzymatic labeling of protein N-termini. Methods Enzymol:in press). Of note, the N-termini of 80-90% of native eukaryotic proteins are acetylated (Polevoda B & Sherman F (2003) N-terminal acetyltransferases and sequence requirements for N-terminal acetylation of eukaryotic proteins. J Mol Biol 325(4):595-622) and therefore do not react with subtiligase. In addition, this approach can be used even in the setting of high-abundance albumin without further depletion or chromatography steps (Gerszten R E, et al. (2008) Challenges in translating plasma proteomics from bench to bedside: update from the NHLBI Clinical Proteomics Programs. Am J Physiol Lung Cell Mol Physiol 295(1):L16-22.). Thus, invention methods allow for high sensitivity and specificity for proteolyic fragments.

In combination with subtiligase labeling a pipeline-based strategy modeled on a previously described approach to identify potential blood-based biomarkers was employed (see, for example, Addona T A, et al. (2011) A pipeline that integrates the discovery and verification of plasma protein biomarkers reveals candidate markers for cardiovascular disease. Nat Biotechnol 29(7):635-643; and Whiteaker J R, et al. (2011) A targeted proteomics-based pipeline for verification of biomarkers in plasma. Nat Biotechnol 29(7):625-634). This strategy first uses a cohort of “high-yield” samples to discover proteomic changes associated with a given condition. Here, using unbiased MS approaches on a QqTOF instrument, proteolytic fragments released from patient tumor and cultured cells were sought post-chemotherapy. The resulting experimental data were combined with an extensive database of proteolytic peptides found during cellular apoptosis, the DegraBase (Crawford E D, et al. (2013) The DegraBase: A Database of Proteolysis in Healthy and Apoptotic Human Cells. Mol Cell Proteomics 12(3):813-824), to develop a targeted “inclusion list” for MS identification on an Orbitrap instrument. This approach allows for the further expansion of the list of proteolytic fragments found in patient samples post-chemotherapy. Finally, targeted, quantitative selected reaction monitoring (SRM) methods were used on a triple-quadrupole instrument (Picotti P & Aebersold R (2012) Selected reaction monitoring-based proteomics: workflows, potential, pitfalls and future directions. Nat Methods 9(6):555-566.) to measure changes in proteolytic N-terminal peptides pre- vs. post-chemotherapy in a larger cohort of patients. The most promising markers increased after chemotherapy administration can be further explored for clinical development.

### Example 18

### Unbiased Discovery MS Combined with N-Terminal Labeling Reveals Numerous Apoptosis-Related Peptides in Patient Plasma Post-Chemotherapy

For discovery samples, a patient cohort with the highest probability of demonstrating proteolytic fragments in the blood post-chemotherapy was sought. Hematologic malignancy patients were identified with circulating malignant cells pre-chemotherapy and a significant drop in these cells (decrease of >7×10⁶ cells/mL blood by hematopathology analysis) within 24 h of initiation of chemotherapy.

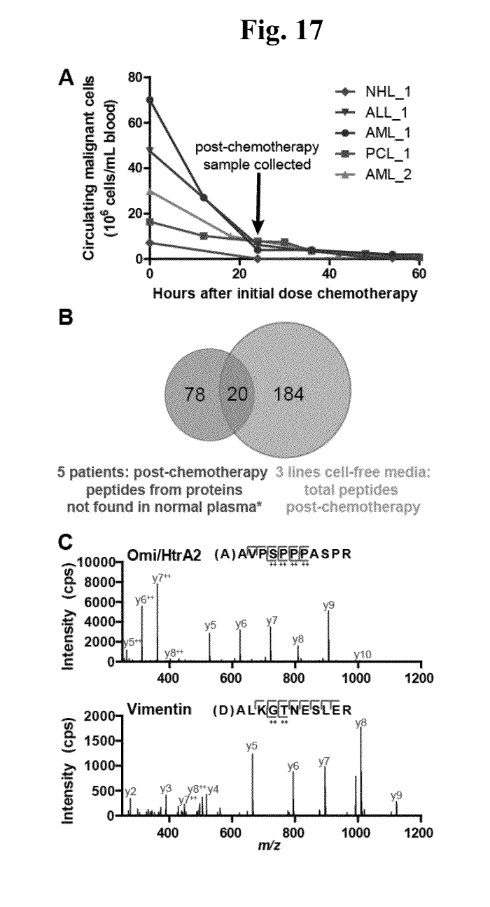

Though patients with these clinical characteristics are relatively rare, 1.5 mL cell-free plasma samples were obtained from five patients (two acute myeloid leukemia, one diffuse large B-cell lymphoma, one B-acute lymphoblastic leukemia, and one multiple myeloma evolved to plasma cell leukemia) (FIG. 17A). N-terminal labeling was performed and reverse phase high-pH fractionation into ten fractions per sample, and evaluated each fraction in data-dependent acquisition mode on a QqTOF MS instrument.

In the post-chemotherapy samples, it was sought to identify proteolytic fragments derived from proteins not found previously in normal blood plasma and serum. It was hypothesized that these new N-termini would be the strongest indicators of release of cleaved intracellular contents into the extracellular medium. Positive results would provide an initial confirmation of the viability of apoptotic biomarker identification. For this comparison to post-chemotherapy samples both a normal plasma sample analyzed here as well as an extensive database of >700 normal blood proteolytic N-terminal peptides previously identified by subtiligase labeling was used (Wildes D & Wells J A (2010) Sampling the N-terminal proteome of human blood. Proc Natl Acad Sci USA 107(10):4561-4566).

Significantly, in each of the five high-yield patient samples between five and 60 N-terminal peptides derived from proteins not found in normal blood were identified, for a total of 98 new peptides across all samples. Remarkably, these peptides demonstrated strong cellular signatures of apoptosis, suggesting that they directly result from chemotherapy effects. For example, these signatures include the mature, processed N-termini from Smac/DIABLO and Omi/HtrA2, which are released from mitochondria to promote caspase activation during apoptosis (Saelens X, et al. (2004) Toxic proteins released from mitochondria in cell death. Oncogene 23(16):2861-2874). The biologically active form of ATF-6, a transcription factor cleaved during cell stress such as that induced by chemotherapy was also identified (Haze K, Yoshida H, Yanagi H, Yura T, & Mori K (1999) Mammalian transcription factor ATF6 is synthesized as a transmembrane protein and activated by proteolysis in response to endoplasmic reticulum stress. Mol Biol Cell 10(11):3787-3799). In addition, numerous peptides with an aspartic acid residue inferred at the P1 position, typical of caspase-cleavage events were also found (see, for example, Crawford E D & Wells J A (2011) Caspase substrates and cellular remodeling. Annu Rev Biochem 80:1055-1087; Mahrus S, et al. (2008) Global sequencing of proteolytic cleavage sites in apoptosis by specific labeling of protein N termini. Cell 134(5):866-876; or Crawford E D, et al. (2013) The DegraBase: A Database of Proteolysis in Healthy and Apoptotic Human Cells. Mol Cell Proteomics 12(3):813-824) Of particular note, multiple caspase-cleaved peptides from the intermediate filament protein vimentin were found. Vimentin, expressed in mesenchymally-derived cells such as leukocytes (Satelli A & Li S (2011) Vimentin in cancer and its potential as a molecular target for cancer therapy. Cell Mol Life Sci 68(18):3033-3046), is analogous to cytokeratin-18 in epithelial cells. In aggregate, in these initial experiments 23 new caspase-cleaved fragments were discovered in the blood whereas only one, derived from cytokeratin-18 (Olofsson M H, et al. (2007) Cytokeratin-18 is a useful serum biomarker for early determination of response of breast carcinomas to chemotherapy. Clin Cancer Res 13(11):3198-3206), was known before. These results provide strong evidence that proteolytically cleaved peptides are directly released into the plasma after chemotherapy and can be identified using our N-terminal labeling method.

### Example 19

### Peptides Released from Cultured Hematologic Malignancy Cells Coincide with Those Found in Post-Chemotherapy Plasma

Previous cellular work focused on identifying caspase-cleaved peptides present in whole cell lysates after induction of apoptosis (see, for example, Mahrus S, et al. (2008) Global sequencing of proteolytic cleavage sites in apoptosis by specific labeling of protein N termini. Cell 134(5):866-876; Crawford E D, et al. (2012) Conservation of caspase substrates across metazoans suggests hierarchical importance of signaling pathways over specific targets and cleavage site motifs in apoptosis. Cell Death Differ 19(12):2040-2048; and Shimbo K, et al. (2012) Quantitative profiling of caspase-cleaved substrates reveals different drug-induced and cell-type patterns in apoptosis. Proc Natl Acad Sci USA 109(31):12432-12437). Here, as a complement to experiments with patient samples, proteolytic products released from cultured cells into the media after chemotherapy were studied. It was reasoned that this system would more closely resemble the physiology of intracellular content release to the plasma in patients treated for blood cancers.

Three cell lines treated with different drugs were evaluated: i) MM1.S, derived from multiple myeloma and treated with the proteasome inhibitor bortezomib, ii) MOLM-13, derived from acute myeloid leukemia and treated with the nucleoside analog cytarabine, and iii) SU-DHL-8, derived from diffuse large B-cell lymphoma and treated with the DNA-damaging agent doxorubicin. All of these conditions reflect the diagnoses of patients in the discovery cohort combined with clinically used chemotherapeutics. Under each condition the cells were either treated with drug or mock-treated for at least 21 h. Treated cells demonstrated at least 50% apoptosis. After removing whole cells, proteins in the media were precipitated with trichloroacetic acid and then subjected to N-terminal labeling by subtiligase. FBS-free media was used in these experiments to avoid contamination from normal bovine plasma proteins.

MS analysis on a QqTOF instrument demonstrated that in all cell types the number of proteolytic fragments in the media post-chemotherapy increased compared to the control samples. Released contents from MM1.S and SU-DHL-8 lines in particular showed strong signatures of apoptosis. For example, the number of released proteolytic fragments with D at P1 sites increased from 3 in the control to 28 post-treatment for MM1.S, and from one to 23 in SU-DHL-8. Across the three cell lines 204 unique N-terminal peptides released into the media post-chemotherapy were identified. Importantly, twenty of these peptides from cell culture experiments were identical to those found in discovery experiments on patient plasma. This remarkable degree of overlap further suggests that the proteolytic fragments in patient plasma are a direct result of intracellular content release after chemotherapy. Notably, the overlapping peptides found in both cultured cells and patient samples included fragments of Smac/DIABLO, Omi/HtrA2, and multiple caspase-cleaved vimentin peptides. These results again support that monitoring proteolytic fragments holds promise as an indicator of cell death post-chemotherapy.

### Example 20

### Targeted Inclusion List Enables Sensitive Detection of Proteolytic Peptides in Post-Chemotherapy Plasma

It was next sought to interrogate the high-yield patient plasma samples for additional proteolytic peptides not initially found by previous unbiased discovery MS of normal blood (Wildes D & Wells J A (2010) Sampling the N-terminal proteome of human blood. Proc Natl Acad Sci USA 107(10):4561-4566). An inclusion list approach on an Orbitrap instrument allows for increased sensitivity of detection for targeted peptides (Addona T A, et al. (2011) A pipeline that integrates the discovery and verification of plasma protein biomarkers reveals candidate markers for cardiovascular disease. Nat Biotechnol 29(7):635-643; Whiteaker J R, et al. (2011) A targeted proteomics-based pipeline for verification of biomarkers in plasma. Nat Biotechnol 29(7):625-634; or Jaffe J D, et al. (2008) Accurate inclusion mass screening: a bridge from unbiased discovery to targeted assay development for biomarker verification. Mol Cell Proteomics 7(10):1952-1962). In this approach, only peptides falling within a narrow mass window around those on the inclusion list were selected for sequencing while other peptides in the sample with masses outside this window, regardless of intensity, were ignored.

To build the targeted inclusion list, the following samples were used i) all peptides found in unbiased discovery experiments on plasma samples, ii) all peptides found released from cultured hematologic malignancy cells post-chemotherapy, and iii) a selection of proteolytic peptides derived from a database of apoptotic samples, the DegraBase (wellslab.ucsf.edu/degrabase; (see also Crawford E D, et al. (2013) The DegraBase: A Database of Proteolysis in Healthy and Apoptotic Human Cells. Mol Cell Proteomics 12(3):813-824). These peptides from the DegraBase included those derived from proteins relevant to apoptosis or cell stress, peptides found to be rapidly cleaved during apoptosis by quantitative MS experiments (Agard N J, et al. (2012) Global kinetic analysis of proteolysis via quantitative targeted proteomics. Proc Natl Acad Sci US A 109(6):1913-1918), and peptides derived from relatively high abundance substrates (Shimbo K, et al. (2012) Quantitative profiling of caspase-cleaved substrates reveals different drug-induced and cell-type patterns in apoptosis. Proc Natl Acad Sci USA 109(31):12432-12437). This strategy, ultimately including 672 peptides, aimed to both confirm peptides already found in plasma as well as identify additional biologically relevant peptides in plasma that were not found earlier.

This inclusion list strategy was implemented on an Orbitrap-based MS instrument to analyze the same five patient samples as used in unbiased discovery experiments. In each of the patient samples between five and 94 proteolytic peptides deriving from proteins not found in normal plasma were identified (Wildes D & Wells J A (2010) Sampling the N-terminal proteome of human blood. Proc Natl Acad Sci USA 107(10):4561-4566), with a total of 140 unique peptides in all. Notably, the targeted inclusion list strategy identified 54 new peptides not found in the unbiased discovery experiments. In addition, a single caspase-cleaved protein fragment had previously been identified in normal plasma (Wildes D & Wells J A (2010) Sampling the N-terminal proteome of human blood. Proc Natl Acad Sci USA 107(10):4561-4566 13). This was derived from gelsolin, an actin-binding protein located at high abundance both intracellularly and in the blood (Bucki R, Levental I, Kulakowska A, & Janmey P A (2008) Plasma gelsolin: function, prognostic value, and potential therapeutic use. Curr Protein Pept Sci 9(6):541-551). This fragment was also included for further study as it was identified in all post-chemotherapy samples.

Combining the results from the targeted and discovery experiments, 153 proteolytic peptides have been identified that represent an initial, novel library of proteolytic biomarkers of cell death for further evaluation (see Table 4). 47 of these peptides (30.7%) demonstrated a D at P1 motif, suggestive of caspase cleavage. This percentage is very similar to the proportion of D at P1 peptides found in typical studies of apoptotic whole cell lysate (see, for example, Crawford E D, et al. (2013) The DegraBase: A Database of Proteolysis in Healthy and Apoptotic Human Cells. Mol Cell Proteomics 12(3):813-824). In addition, based on protein expression data in the PaxDB database (Wang M, et al. (2012) PaxDb, a database of protein abundance averages across all three domains of life. Mol Cell Proteomics 11(8):492-500), 142 (92.8%) of these peptides are derived from proteins that are typically present intracellularly rather than in the blood. The methods described herein could sensitively detect in the cell-free plasma many proteins typically present at <10 ppm intracellularly. These cumulative results further support the notion that the methods described herein are detecting the release of intracellular contents post-chemotherapy.

Table 4 presents the novel proteolytic peptides that have been identified herein.

TABLE 4

| | | | | P1 |
|---|---|---|---|---|
| SEQ | | | | position |
| ID | | Uniprot. | | amino |
| NO | Sequence | Acc. No. | Protein | acid |
| 439 | AITELEDAFSR | Q6DN90 | IQ motif and SEC7 | D |
| | | | domain-containing | |
| | | | protein 1 | |
| 440 | AAAVAVPLAGGQEGSPGGGR | Q92541 | RNA polymerase- | A |
| | | | associated protein | |
| | | | RTF1 homolog | |
| 441 | STFYLGER | P00450 | Ceruloplasmin | D |
| 442 | GFSAKEAQDTSDGIIQK | P18850 | Cyclic AMP- | L |
| | | | dependent | |
| | | | transcription factor | |
| | | | ATF-6 alpha | |
| 443 | VTAMDVVYALKR | P62805 | Histone H4 | T |
| 444 | SYPARVPPPPPIAR | P07910 | Heterogeneous | Y |
| | | | nuclear | |
| | | | ribonucleoproteins | |
| | | | C1/C2 | |
| 445 | SYELPDGQVITIGNER | P60709 | Actin, cytoplasmic 1 | K |
| 446 | SVYYNEATGGKYVPR | P07437 | Tubulin beta chain | I |
| 447 | SVPRGEAAGAVQELAR | Q9UHG2 | ProSAAS | R |
| 448 | SLTTIPELKDHLR | Q86UX7 | Fermitin family | D |
| | | | homolog 3 | |
| 449 | SLTPAVPVESKPDKPSGK | P20810 | Calpastatin | K |
| 450 | SLQSVAEER | P61313 | 60S ribosomal | R |
| | | | protein L15 | |
| 451 | SLPGEQEQEVAGSK | Q04721 | Neurogenic locus | R |
| | | | notch homolog | |
| | | | protein 2 | |
| 452 | SKLNYKPPPQK | P52566 | Rho GDP- | D |
| | | | dissociation | |
| | | | inhibitor 2 | |
| 453 | SISSQLGPIHPPPR | Q92945 | Far upstream | D |
| | | | element-binding | |
| | | | protein 2 | |
| 454 | SHHAASTTTAPTPAAR | Q7Z6Z7 | E3 ubiquitin- | R |
| | | | protein ligase | |
| | | | HUWE1 | |
| 455 | SGPPVSELITK | P10412 | Histone H1.4 | A |
| 456 | SFQTSPSTESLK | P98171 | Rho GTPase- | D |
| | | | activating protein 4 | |
| 457 | SFPTQDHLPATPR | Q13275 | Semaphorin-3F | P |
| 458 | SFGGDAQADEGQAR | P33992 | DNA replication | D |
| | | | licensing factor | |
| | | | MCM5 | |
| 459 | SFFTPGKPK | Q9NTI5 | Sister chromatid | K |
| | | | cohesion protein | |
| | | | PDS5 homolog B | |
| 460 | SAVGTLPATSPQSTSVQAK | Q13428 | Treacle protein | D |
| 461 | SAPGGGSKVPQK | P06748 | Nucleophosmin | R |
| 462 | SAPATGGVKKPHR | P68431 | Histone H3.1 | K |
| 463 | NSPSTTPPTVTTNMPVTNR | Q86SQ4 | G-protein coupled | V |
| | | | receptor 126 | |
| 464 | MWISKQEYDESGPSIVHR | P60709 | Actin, cytoplasmic 1 | Q |
| 465 | MVSPFHSPPSTPSSPGVR | Q6JBY9 | CapZ-interacting | A |
| | | | protein | |
| 466 | MIASDSHRPEVK | Q9NYF8 | Bcl-2-associated | K |
| | | | transcription factor 1 | |
| 467 | MAPVPLDDSNRPASLTKDR | Q9NYF8 | Bcl-2-associated | K |
| | | | transcription factor 1 | |
| 468 | MVLLESEQFLTELTR | P37108 | Signal recognition | — |
| | | | particle 14 kDa | |
| | | | protein | |
| 469 | LVQSPNSYFMDVK | P42677 | 40S ribosomal | R |
| | | | protein S27 | |
| 470 | LQSAHPGEHLAQGASR | Q9UEW3 | Macrophage | L |
| | | | receptor MARCO | |
| 471 | KASGPPVSELITK | P10412 | Histone H1.4 | R |
| 472 | IWHHTFYNELR | P60709 | Actin, cytoplasmic 1 | K |
| 473 | GVTQFGNKYIQQTKPLTLER | O43809 | Cleavage and | R |
| | | | polyadenylation | |
| | | | specificity factor | |
| | | | subunit 5 | |
| 474 | GVPSDSVEAAKNASNTEK | Q99733 | Nucleosome | D |
| | | | assembly protein 1- | |
| | | | like 4 | |
| 475 | GVPLDATEDSKKNEPIFK | O15042 | U2 snRNP- | D |
| | | | associated SURP | |
| | | | motif-containing | |
| | | | protein | |
| 476 | GVAATPGKAEATR | Q7Z591 | AT-hook- | D |
| | | | containing | |
| | | | transcription factor | |
| 477 | GSSPLLDIVGGR | P20160 | Azurocidin | A |
| 478 | GSETPQLFTVLPEKR | Q13435 | Splicing factor 3B | D |
| | | | subunit 2 | |
| 479 | GLPEEQPQTTK | Q7Z6Z7 | E3 ubiquitin- | D |
| | | | protein ligase | |
| | | | HUWE1 | |
| 480 | GLLPTPDEFPR | Q9C0J8 | pre-mRNA 3′ end | K |
| | | | processing protein | |
| | | | WDR33 | |
| 481 | GLGVARPHYGSVLDNER | P46940 | Ras GTPase- | D |
| | | | activating-like | |
| | | | protein IQGAP1 | |
| 482 | GISAGAVQTAGK | P46087 | Putative ribosomal | K |
| | | | RNA | |
| | | | methyltransferase | |
| | | | NOP2 | |
| 483 | GINYQPPTVVPGGDLAK | P68363 | Tubulin alpha-1B | V |
| | | | chain | |
| 484 | GGGPGQVVDDGLEHR | Q8WUI4 | Histone | D |
| | | | deacetylase 7 | |
| 485 | GFFWTQGSPKPGTASPK | Q86YV5 | Tyrosine-protein | D |
| | | | kinase SgK223 | |
| 486 | FVSEAELDER | Q9GZU8 | Protein FAM192A | R |
| 487 | FFSALEK | Q76I76 | Protein | D |
| | | | phosphatase | |
| | | | Slingshot homolog 2 | |
| 488 | AYEPQGGSGYDYSYAGGR | P61978 | Heterogeneous | M |
| | | | nuclear | |
| | | | ribonucleoprotein K | |
| 489 | AVPKEDIYSGGGGGGSR | Q13151 | Heterogeneous | K |
| | | | nuclear | |
| | | | ribonucleoprotein | |
| | | | A0 | |
| 490 | AVPIAQKSEPHSLSSEALMR | Q9NR28 | Diablo homolog, | C |
| | | | mitochondrial | |
| 491 | AVFPSIVGRPR | P60709 | Actin, cytoplasmic 1 | R |
| 492 | ATVTPSPVKGK | Q9H1E3 | Nuclear ubiquitous | K |
| | | | casein and cyclin- | |
| | | | dependent kinases | |
| | | | substrate | |
| 493 | ASSASSFLDSDELER | Q14498 | RNA-binding | D |
| | | | protein 39 | |
| 494 | AMEELDGDDVRVSSR | Q8IYJ1 | Copine-9 | E |
| 495 | ALYVACQGQPK | O14686 | Histone-lysine N- | D |
| | | | methyltransferase | |
| | | | MLL2 | |
| 496 | ALPSHLGLHPER | P78325 | Disintegrin and | R |
| | | | metalloproteinase | |
| | | | domain-containing | |
| | | | protein 8 | |
| 497 | ALLNLPGTQTSGEAK | Q96GM8 | Target of EGR1 | R |
| | | | protein 1 | |
| 498 | ALIGDDVGLTSYKHR | Q53F19 | Uncharacterized | R |
| | | | protein C17orf85 | |
| 499 | AITGASLADIMAKR | P83731 | 60S ribosomal | R |
| | | | protein L24 | |
| 500 | AAGYDVEKNNSR | P10412 | Histone H1.4 | A |
| 501 | FVVPVASPSGDAR | P21333 | Filamin-A | P |
| 502 | AAAGAPLPR | O14745 | Na(+)/H(+) | D |
| | | | exchange | |
| | | | regulatory cofactor | |
| | | | NHE-RF1 | |
| 503 | GLGPQGFPELKNDTFLR | P06132 | Uroporphyrinogen | N |
| | | | decarboxylase | |
| 504 | AAPEEESAYVAGEKR | Q9UNZ2 | NSFL1 cofactor | G |
| | | | p47 | |
| 505 | SLPEAGPGR | P35318 | ADM | R |
| 506 | GLSPLSSPSDTK | Q9UKV3 | Apoptotic | G |
| | | | chromatin | |
| | | | condensation | |
| | | | inducer in the | |
| | | | nucleus | |
| 507 | AAGVTDGNEVAK | Q9BX68 | Histidine triad | G |
| | | | nucleotide-binding | |
| | | | protein 2, | |
| | | | mitochondrial | |
| 508 | VFIGINDLEK | Q9BWP8 | Collectin-11 | R |
| 509 | AITGASLADIMAK | P83731 | 60S ribosomal | R |
| | | | protein L24 | |
| 510 | GIVPDIAVGTKR | P26599 | Polypyrimidine | D |
| | | | tract-binding | |
| | | | protein 1 | |
| 511 | AASQLNVDASGNLAK | Q9NZL9 | Methionine | D |
| | | | adenosyltransferase | |
| | | | 2 subunit beta | |
| 512 | MLDDIVSR | Q92945 | Far upstream | M |
| | | | element-binding | |
| | | | protein 2 | |
| 513 | GFDVASVQQQR | O60664 | Perilipin-3 | D |
| 514 | AAPELPVPTGGPAVGAR | P21281 | V-type proton | G |
| | | | ATPase subunit B, | |
| | | | brain isoform | |
| 515 | SVVSFDKVKEPR | Q15424 | Scaffold | R |
| | | | attachment factor | |
| | | | B1 | |
| 516 | GLAVTPTPVPVVGSQMTR | P26368 | Splicing factor | D |
| | | | U2AF 65 kDa | |
| | | | subunit | |
| 517 | ALAEGPGAEGPR | Q13263 | Transcription | M |
| | | | intermediary factor | |
| | | | 1-beta | |
| 518 | GQSDENKDDYTIPDEYR | P43243 | Matrin-3 | D |
| 519 | GFAEAIHSPQVAGVPR | P12270 | Nucleoprotein TPR | D |
| 520 | AVPSPPPASPR | O43464 | Serine protease | A |
| | | | HTRA2, | |
| | | | mitochondrial | |
| 521 | GISSSNEGVEEPSKKR | Q9NXV6 | CDKN2A- | R |
| | | | interacting protein | |
| 522 | GIGTVPVGR | P68104 | Elongation factor | G |
| | | | 1-alpha 1 | |
| 523 | ALPGDNVGFNVK | P68104 | Elongation factor | E |
| | | | 1-alpha 1 | |
| 524 | GLVETPTGYIESLPR | P55209 | Nucleosome | D |
| | | | assembly protein 1- | |
| | | | like 1 | |
| 525 | SLLEPRDPVASSLSPYFGTK | Q9UNW1 | Multiple inositol | C |
| | | | polyphosphate | |
| | | | phosphatase 1 | |
| 526 | SISESVPVGPKVR | P45974 | Ubiquitin | D |
| | | | carboxyl-terminal | |
| | | | hydrolase 5 | |
| 527 | SISESVPVGPK | P45974 | Ubiquitin | D |
| | | | carboxyl-terminal | |
| | | | hydrolase 5 | |
| 528 | GVPSDSVEAAK | Q99733 | Nucleosome | D |
| | | | assembly protein 1- | |
| | | | like 4 | |
| 529 | AINTEFK | P08670 | Vimentin | D |
| 530 | SLADAINTEFKNTR | P08670 | Vimentin | F |
| 531 | ASGPPVSELITK | P10412 | Histone H1.4 | K |
| 532 | ALAAAGYDVEK | P10412 | Histone H1.4 | K |
| 533 | ALAAAGYDVEKNNSR | P10412 | Histone H1.4 | K |
| 534 | GVTHTVPIYEGYALPHAILR | P60709 | Actin, cytoplasmic 1 | D |
| 535 | GMGQKDSYVGDEAQSKR | P60709 | Actin, cytoplasmic 1 | V |
| 536 | GFAGDDAPR | P60709 | Actin, cytoplasmic 1 | A |
| 537 | LLTEAPLNPK | P60709 | Actin, cytoplasmic 1 | V |
| 538 | AGFAGDDAPR | P60709 | Actin, cytoplasmic 1 | K |
| 539 | VAPEEHPVLLTEAPLNPK | P60709 | Actin, cytoplasmic 1 | R |
| 540 | ALDFEQEMATAASSSSLEK | P60709 | Actin, cytoplasmic 1 | V |
| 541 | GASQFQEVIR | Q16851 | UTP--glucose-1- | D |
| | | | phosphate | |
| | | | uridylyltransferase | |
| 542 | ALGSPEMDVR | O15446 | DNA-directed | M |
| | | | RNA polymerase I | |
| | | | subunit RPA34 | |
| 543 | GMTELEPSKFSK | Q9ULF5 | Zinc transporter | R |
| | | | ZIP10 | |
| 544 | GLPTGAEGR | Q12906 | Interleukin | M |
| | | | enhancer-binding | |
| | | | factor 3 | |
| 545 | GFDQNVNVK | P43686 | 26S protease | D |
| | | | regulatory subunit | |
| | | | 6B | |
| 546 | YIPAENSPTR | Q86XP3 | ATP-dependent | P |
| | | | RNA helicase | |
| | | | DDX42 | |
| 547 | SISESAFSAR | O75487 | Glypican-4 | R |
| 548 | AAVQAAILSGDK | P11142 | Heat shock cognate | G |
| | | | 71 kDa protein | |
| 549 | ALLQTDQSLSEKEK | P32456 | Interferon-induced | D |
| | | | guanylate-binding | |
| | | | protein 2 | |
| 550 | ALAAGGYDVEKNNSR | P16401 | Histone H1.5 | K |
| 551 | GSSPLLDIVGGRK | P20160 | Azurocidin | A |
| 552 | AVPIAQK | Q9NR28 | Diablo homolog, | C |
| | | | mitochondrial | |
| 553 | SIFQHIQSAQSQR | Q9Y2W1 | Thyroid hormone | R |
| | | | receptor-associated | |
| | | | protein 3 | |
| 554 | SLRPDPNFDALISK | Q06587 | E3 ubiquitin- | R |
| | | | protein ligase | |
| | | | RING1 | |
| 555 | EHGLAPAPTTIR | F5GYI3 | Ubiquitin- | P |
| | | | associated protein | |
| | | | 1-likeP | |
| 556 | WTYHYSEKPMNWQR | P14151 | L-selectin | C |
| 557 | GLLLLGSGSR | Q9Y662 | Heparan sulfate | F |
| | | | glucosamine 3-O- | |
| | | | sulfotransferase | |
| | | | 3B1 | |
| 558 | SVPAAEPEYPKGIR | P54819 | Adenylate kinase | P |
| | | | 2, mitochondrial | |
| 559 | SVPAAEPEYPK | P54819 | Adenylate kinase | P |
| | | | 2, mitochondrial | |
| 560 | GLGLSYLSSHIANVER | P06396 | Gelsolin | D |
| 561 | AINTEFKNTR | P08670 | Vimentin | D |
| 562 | ALKGTNESLER | P08670 | Vimentin | D |
| 563 | AEIVGGHEAQPHSRPYMASLQMR | P24158 | Myeloblastin | A |
| 564 | SMPPAQQQITSGQMHR | Q9Y490 | Talin-1 | G |
| 565 | MVMEKPSPLLVGR | Q13283 | Ras GTPase- | — |
| | | | activating protein- | |
| | | | binding protein 1 | |
| 566 | MMLDDIVSR | Q92945 | Far upstream | K |
| | | | element-binding | |
| | | | protein 2 | |
| 567 | MKETIMNQEK | P20290 | Transcription | Q |
| | | | factor BTF3 | |
| 568 | MVMAEGTAVLRR | Q9Y3A3 | MOB-like protein | — |
| | | | phocein | |
| 569 | AMLDQLMGTSR | Q9Y383 | Putative RNA- | R |
| | | | binding protein | |
| | | | Luc7-like 2 | |
| 570 | MVNFTVDQIR | P13639 | Elongation factor 2 | — |
| 571 | MGLLSQGSPLSWEETKR | P48506 | Glutamate-- | — |
| | | | cysteine ligase | |
| | | | catalytic subunit | |
| 572 | MGVQVETISPGDGR | P62942 | Peptidyl-prolyl cis- | — |
| | | | trans isomerase | |
| | | | FKBP1A | |
| 573 | AYFEKVGDTSLDPNDFDFTVTGRGS | P51608 | Methyl-CpG- | I |
| | PSR | | binding protein 2 | |
| 574 | VFDNGSIYNPEVLDITEETLHSR | P05388 | 60S acidic | Q |
| | | | ribosomal protein | |
| | | | P0 | |
| 575 | SIGASPNPFSVHTATAVPSGK | P09884 | DNA polymerase | R |
| | | | alpha catalytic | |
| | | | subunit | |
| 576 | KVDEGAGDSAAVASGGAQTLALAG | Q9NZT2 | Opioid growth | R |
| | SPAPSGHPK | | factor receptor | |
| 577 | GSDASQLLHQAEVAQQEFLEVK | Q96PK2 | Microtubule-actin | D |
| | | | cross-linking factor | |
| | | | 1, isoform 4 | |
| 578 | AVTPGPQPTLEQLEEGGPRPLER | Q27J81 | Inverted formin-2 | D |
| 570 | AVSGQLPDPTTNPSAGKDGPSLLVV | Q8N1G4 | Leucine-rich | D |
| | EQVR | | repeat-containing | |
| | | | protein 47 | |
| 580 | ALVEFESNPEETREPGSPPSVQR | Q9H6F5 | Coiled-coil | R |
| | | | domain-containing | |
| | | | protein 86 | |
| 581 | GVPVPGSPFPLEAVAPTKPSK | P21333 | Filamin-A | D |
| 582 | GQHPAQEEVPESPQTSGPEAENR | Q6JBY9 | CapZ-interacting | D |
| | | | protein | |
| 583 | SAHPEEGDLDLASESTAHAQSSK | Q15424 | Scaffold | D |
| | | | attachment factor | |
| | | | B1 | |
| 584 | GVPSDSVEAAKNASNTEKLTDQVM | Q99733 | Nucleosome | D |
| | QNPR | | assembly protein 1- | |
| | | | like 4 | |
| 585 | AAPAPAPPPEPERPKEVEFDASK | P08590 | Myosin light chain 3 | K |
| 586 | ATVGGPAPTPLLPPSATASVK | Q07666 | KH domain- | D |
| | | | containing, RNA- | |
| | | | binding, signal | |
| | | | transduction- | |
| | | | associated protein 1 | |
| 587 | GVQLPPGDYSTTPGGTLFSTTPGGTR | Q13541 | Eukaryotic | D |
| | | | translation | |
| | | | initiation factor 4E- | |
| | | | binding protein 1 | |
| 588 | SLAGSSGPGASSGTSGDHGELVVR | P29692 | Elongation factor | K |
| | | | 1-delta | |
| 589 | SFSDADLADGVSGGEGK | P14209 | CD99 antigen | G |
| 590 | SSASSGPQILK | P08648 | Integrin alpha-5 | R |
| 591 | LYQTIEENIK | Q9Y287 | Integral membrane | A |
| | | | protein 2B | |

### Example 21

### A Quantitative Proteomic Assay Demonstrates Increases in Proteolytic Fragment Abundance Post- Vs. Pre-Chemotherapy

If these markers of proteolysis are to be useful in a diagnostic context, they must distinguish relative increases in proteolytic fragments after chemotherapy compared to before. Targeted SRM methods were therefore used on a triple-quadrupole instrument to quantitatively measure these fragments. SRM allows for highly sensitive, label-free quantification of peptides by monitoring the intensity and LC co-elution of targeted parent ion/fragment ion pairs (“transitions”) (Picotti P & Aebersold R (2012) Selected reaction monitoring-based proteomics: workflows, potential, pitfalls and future directions. Nat Methods 9(6):555-566). To develop accurate SRM assays, crude spot-synthesized peptides were first sequenced by LC-MS/MS for 121 of the 153 targets in the library. The remaining peptides either could not be synthesized by this method or were not detected by LC-MS/MS. 117 (96.6%) of these synthetic peptides demonstrated similar MS/MS spectra and LC retention times to those identified in plasma, suggesting a high rate of true positive identification in plasma experiments. Importantly, as others have previously shown (Picotti P, et al. (2010) High-throughput generation of selected reaction-monitoring assays for proteins and proteomes. Nat Methods 7(1):43-46), these synthetic peptides allowed for the development of higher-quality SRM assays: for each well-characterized peptide the most intense fragment ions and LC retention time can be obtained directly on the triple-quadrupole instrument. For the remaining peptides SRM assays were developed by selecting co-eluting peptide transitions in either plasma or cell culture samples, similar to that done previously (see, for example, Shimbo K, et al. (2012) Quantitative profiling of caspase-cleaved substrates reveals different drug-induced and cell-type patterns in apoptosis. Proc Natl Acad Sci USA 109(31):12432-12437; or Wiita A P, et al. (2013) Global cellular response to chemotherapy-induced apoptosis. Elife 2:e01236). Overall, SRM assays were successfully developed for 140 of the 153 peptides of interest.

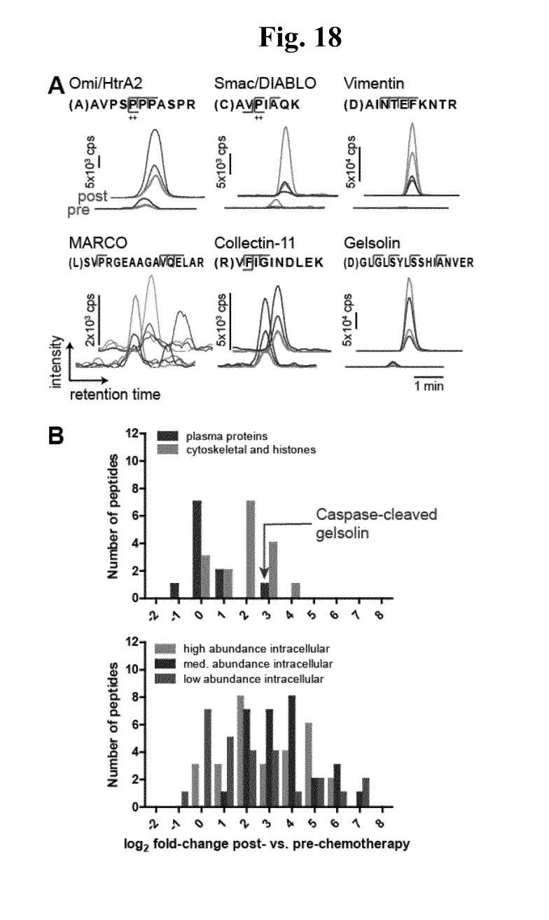

This completed SRM method was next applied to hematologic malignancy patient samples. As an initial case, the only “high-yield” post-chemotherapy patient sample that also had a paired pre-treatment sample (AML_{—}1 in FIG. 17A) was studied. N-terminal enrichment to 500 μL of plasma was applied at each time point and the unfractionated peptides analyzed by SRM in duplicate with intensity normalization by spike-in protein standards. In the post-chemotherapy sample 100 of the 140 peptides (71.4%) were detected with intensity signal above baseline noise. More importantly, by total peak area intensity, 90 of these peptides were increased in abundance post- vs. pre-chemotherapy, with 77 showing at least a 2-fold increase. Fragments from typically intracellular proteins highly increased post-chemotherapy included the N-termini of Smac/DIABLO and Omi/HtrA2 as well as caspase-cleaved fragments of vimentin (FIG. 18A). In contrast, of the 10 detected peptides derived from proteins typically found at high abundance in normal plasma (from PaxDB analysis), 9 showed little change in abundance (FIGS. 18A, MARCO and Collectin-11, and 18B, upper panel). The only exception was the caspase-cleaved fragment of gelsolin, which showed an 8.5-fold increase after treatment. In contrast to these results from typical plasma proteins, peptides arising from intracellular proteins showed a wide range of abundance increases post-chemotherapy, some over 50-fold. These results firmly demonstrate that the appearance of proteolytic fragments in the plasma is indicative of post-chemotherapy apoptosis.

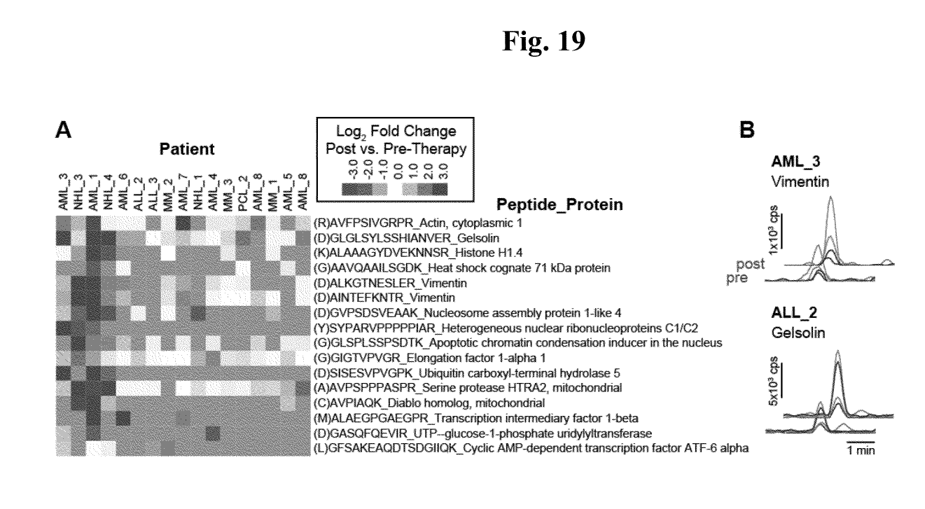

Pre- and post-chemotherapy plasma samples were collected from another 16 hematologic malignancy patients for additional quantitative validation. Post-chemotherapy samples were collected between 12-96 h after initiation of treatment. Patients ranged in diagnosis, severity of disease, treatment regimen, and degree of response. The above-described SRM method was applied to these patient samples and changes pre- and post-chemotherapy were examined. In FIG. 19A, 16 peptides with increased post-chemotherapy across multiple patients are displayed, with examples of −2-fold increases from two patients in FIG. 19B. Overall, these peptides represent the most promising targets for further exploration in clinical development as biomarkers of chemotherapeutic efficacy.

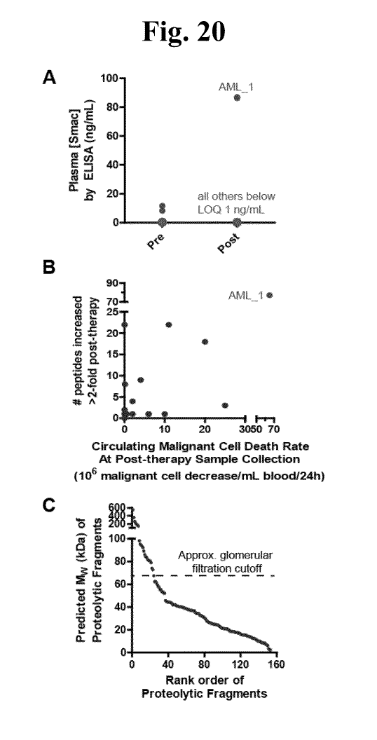

It was then sought to confirm the quantitative proteomic results by an independent method. Though specific antibodies are not available for the endoproteolytic fragments found herein to be increased post-chemotherapy, it was possible to use a sandwich ELISA towards the protein Smac. In this protein the N-terminus of the intact, mature protein was monitored without any further endoproteolysis. While for most patients the levels of Smac fell below the ELISA limit of quantification, for the patient AML_{—}1 Smac was positively identified in both the pre- and post-chemotherapy samples. Notably, the measured abundance increase, from 10 ng/mL pre- to 86 ng/mL post-treatment (FIG. 20A), is directly in line with the 8.5-fold increase measured by SRM.

Here it has been demonstrated that specific enzymatic labeling of protein N-termini, combined with a combination of unbiased and targeted MS approaches, reveals that many more proteolytic fragments are released from dying tumor cells than were previously known. The experimental approach described herein allowed for the identification of such fragments, which could not be detected by typical plasma proteomic methods. The further use of quantitative MS approaches show that many of these proteolytic N-termini are increased within days of chemotherapy initiation across multiple blood cancer patients. These results describe a promising initial set of novel, rapid, and potentially inexpensive set of protein-based biomarkers of chemotherapeutic efficacy.

Through targeted quantitative proteomics, it has been found that a greater rate of malignant cell decrease in the peripheral blood post-chemotherapy correlates with a greater number of increased proteolytic fragments (FIG. 20B). This finding, along with the overlap in results between cultured tumor cells and patient samples, suggests the proteolytic fragments identified herein correspond to death of tumor cells and not normal somatic cells. One of the surprising findings in this study is the high degree of patient-to-patient variability in proteolytic peptides identified post-chemotherapy. In the high-yield samples, 5- to 10-fold more peptides post-chemotherapy were identified from the patients AML_{—}1 and NHL_{—}1 (Table S1) compared to the other three patients, even though the other three (ALL_{—}1, PCL 1, and AML_{—}2) also demonstrated large, rapid decreases in circulating malignant cell count (FIG. 17A). Large variability was also observed in the quantitative SRM assay (FIG. 19A). There are many potential reasons for this observation. It is possible that decreases in circulating malignant cells do not always reflect apoptosis occurring directly in the blood. Instead, it could be a reflection of tumor cells partitioning away from the blood and toward the bone marrow or lymph nodes but without death.

Alternatively, the mechanism by which intracellular contents are released is still unknown. Release may only occur when normal phagocytic functions of macrophages, which typically sequester cellular fragments generated during apoptosis, become overwhelmed. Therefore there may be patient-to-patient variation in the threshold where intracellular content release occurs. Furthermore, these patients had different diagnoses, different disease burdens, and were treated with different drugs. There may be disease- or drug-specific tumor effects which govern the release of these contents.

Another important issue which likely governs proteolytic fragment detection is renal clearance. It is well-known that proteins with molecular weight below that of serum albumin (69.4 kDa) are rapidly filtered through the renal glomeruli (Lote C J (2012) Principles of Renal Physiology (Springer, New York); 5 Ed.). For the 153 proteolytic fragments studied here, extending from the identified cleavage site to the protein C-terminus, the large majority are predicted to be below this size cutoff (FIG. 20C). Therefore there may only be a short time window between the induction of apoptosis in a tumor and renal excretion of these proteolytic fragments.

It is encouraging to note that it was possible herein to detect many new caspase-derived peptides post-chemotherapy when to date the only validated product is the caspase-cleaved peptide from cytokeratin-18 (Olofsson M H, et al. (2007) Cytokeratin-18 is a useful serum biomarker for early determination of response of breast carcinomas to chemotherapy. Clin Cancer Res 13(11):3198-3206). This finding also highlights the ability of the N-terminal enrichment technology described herein to identify proteolytic fragments not previously found by traditional plasma proteomics.

To demonstrate the feasibility of invention methods, hematologic cancers were initially studied, where cell death occurs directly in communication with the blood compartment. Further improvements in mass spectrometer sensitivity may allow for the identification of specific proteolytically-cleaved fragments in the blood of patients treated for solid tumors. One of the ultimate goals herein is to evaluate the clinical utility of monitoring proteolytic fragments post-chemotherapy, which will require larger-scale human studies. These studies will preferably take place in patients with a single disease and treated with similar apoptosis-inducing regimens. Important endpoints, which were not possible to evaluate rigorously in the heterogeneous samples employed herein, include whether increases in proteolytic fragments correlate with other measures of therapeutic efficacy (bone marrow biopsy, PET/CT scans, etc.) as well as patient overall survival.

Larger-scale trials will require the development of medium- to high-throughput assays using antibodies specific for the proteolytic fragment of interest, as the current N-terminomic method is not suited to evaluation of hundreds of samples from dozens of patients. Of note, the SRM results and Smac ELISA experiments described herein indicate that antibody-based assays may have to be highly sensitive and specific in order to detect potentially small (<1 ng/mL) changes in protein levels post-treatment. Ideally, for use in clinical testing these antibodies would be appropriate for panel-based, MS-free approaches, either by traditional ELISA or automated immunoassays. It is envisioned that this diagnostic test would be used pre-treatment and post-treatment to monitor increases in apoptosis. It is unlikely that there would be a single absolute cutoff value to determine therapeutic efficacy in the general population, as different patient tumors will demonstrate different baseline levels of apoptosis (Milross C G, et al. (1996) Relationship of mitotic arrest and apoptosis to antitumor effect of paclitaxel. J Natl Cancer Inst 88(18):1308-1314). Therefore, the relative change after treatment for an individual patient, as monitored herein by SRM, will be more clinically relevant.

Overall, the results presented herein demonstrate the promise of monitoring proteolysis post-chemotherapy as a strategy to rapidly determine chemotherapeutic efficacy. The results presented herein greatly expand the potential repertoire of circulating markers of apoptosis beyond the few already known. These markers would have great utility in early-stage studies of new anti-cancer compounds or other therapeutics which lead to apoptotic cell death. Alternatively, proteolytic fragments found to be specific for death of normal bone marrow or gastrointestinal tissues could serve as new markers of toxicity for an array of drug treatments. By applying similar methods to other tumor types and in larger patient cohorts, it may be possible to identify an entirely new class of general, cancer type- or drug-specific biomarkers of therapeutic efficacy. Such diagnostic tests would represent an important advance toward the goal of personalized therapeutic regimens.

It is understood that the examples and embodiments described herein are for illustrative purposes only and that various modifications or changes in light thereof will be suggested to persons skilled in the art and are to be included within the spirit and purview of this application and scope of the appended claims. All publications, accession numbers, patents, and patent applications cited herein are hereby incorporated by reference in their entirety for all purposes.

What is claimed is:

1. A method of determining a level of a peptide in a cancer subject, the method comprising:
(i) assaying a biological sample from a cancer subject; and
(ii) determining a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in said biological sample.

2. The method of claim 1, wherein said determining comprises contacting a binding reagent with said peptide to form a binding reagent peptide complex.

3. The method of claim 2, wherein said binding reagent is an antibody or an aptamer.

4. The method of claim 1, wherein said subject has a hematologic malignancy.

5. The method of claim 1, wherein said subject is receiving or has received a therapeutic agent.

6. The method of claim 5, wherein said therapeutic agent is a chemotherapeutic agent, a radiotherapeutic agent, an apoptosis inducing agent or a cytotoxic agent.

7. A method of determining apoptosis in a subject, the method comprising:
(i) detecting a level of a peptide of SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552 in a biological sample from a subject receiving or having received a therapeutic agent, wherein said detecting comprises contacting a binding reagent with the peptide to form a binding reagent peptide complex and detecting the binding reagent peptide complex; and
(ii) comparing said level to a standard control, thereby determining apoptosis in a subject.

8. The method of claim 7, wherein said subject has cancer.

9. The method of claim 7, wherein said therapeutic agent is a chemotherapeutic agent, a radiotherapeutic agent, an apoptosis inducing agent or a cytotoxic agent.

10. An in vitro polypeptide complex comprising a peptide bound to a binding reagent, wherein said peptide is SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552.

11. The complex of claim 10, wherein said binding reagent is an antibody or an aptamer.

12. The complex of claim 11, wherein said antibody comprises a detectable moiety.

13. The complex of claim 10, wherein said binding reagent is bound to a solid support, wherein said solid support comprises glass, plastic, ceramic, modified silica, nylon or quartz.

14. A conjugate comprising a peptide covalently bound to a detectable moiety, wherein said peptide is SEQ ID NO:442, SEQ ID NO:444, SEQ ID NO:491, SEQ ID NO:506, SEQ ID NO:517, SEQ ID NO:520, SEQ ID NO:522, SEQ ID NO:527, SEQ ID NO:533, SEQ ID NO:541, SEQ ID NO:548 or SEQ ID NO:552.

15. The conjugate of claim 14, wherein said peptide is bound to a binding reagent.

16. The conjugate of claim 15, wherein said binding reagent is an antibody or an aptamer.

17. The conjugate of claim 14, wherein said detectable moiety is attached to a solid support.


SEQUENCE LISTING (591 sequences)

SEQ ID NO: 1 - protein, 22 aa, Homo sapiens
AAQAPAEQPHSSSDAAQAPCPR

SEQ ID NO: 2 - protein, 10 aa, Homo sapiens
AVAEPANAVK

SEQ ID NO: 3 - protein, 14 aa, Homo sapiens
AVAEPANAVKGAGK

SEQ ID NO: 4 - protein, 17 aa, Homo sapiens
AVAEPANAVKGAGKEMK

SEQ ID NO: 5 - protein, 23 aa, Homo sapiens
GLVSPSNNSKEDAFSGTDWMLEK

SEQ ID NO: 6 - protein, 14 aa, Homo sapiens
GSGMLDEDEEDLQR

SEQ ID NO: 7 - protein, 11 aa, Homo sapiens
SVTDTEPEDEK

SEQ ID NO: 8 - protein, 17 aa, Homo sapiens
SVTDTEPEDEKVVSYSK

SEQ ID NO: 9 - protein, 25 aa, Homo sapiens
YVVPVEDNDENYIHPTESSSPPPEK

SEQ ID NO: 10 - protein, 21 aa, Homo sapiens
MHGDSEYNIMFGPDICGPGTK

SEQ ID NO: 11 - protein, 14 aa, Homo sapiens
GSDVVSDLEHEEMK

SEQ ID NO: 12 - protein, 12 aa, Homo sapiens
GGPPCAPGGTAK

SEQ ID NO: 13 - protein, 41 aa, Homo sapiens
GFQSESPEKLDPVEQGQEDTVAPEVAAEKPVGALLGPGAER

SEQ ID NO: 14 - protein, 19 aa, Homo sapiens
GGGDNKEGEDSSVIHYDDK

SEQ ID NO: 15 - protein, 23 aa, Homo sapiens
GGGDNKEGEDSSVIHYDDKAIER

SEQ ID NO: 16 - protein, 18 aa, Homo sapiens
GFYMEDGDPSVAQLLHER

SEQ ID NO: 17 - protein, 10 aa, Homo sapiens
GTLSTIEFQR

SEQ ID NO: 18 - protein, 25 aa, Homo sapiens
GSLGDDFDANDEPDHTAVGDHEEFR

SEQ ID NO: 19 - protein, 16 aa, Homo sapiens
GFVEATEGLGDDALGK

SEQ ID NO: 20 - protein, 10 aa, Homo sapiens
LFGTTDAVVK

SEQ ID NO: 21 - protein, 19 aa, Homo sapiens
GVADSTVISSMPCLLMELR

SEQ ID NO: 22 - protein, 20 aa, Homo sapiens
GVADSTVISSMPCLLMELRR

SEQ ID NO: 23 - protein, 14 aa, Homo sapiens
GKGQEVETSVTYYR

SEQ ID NO: 24 - protein, 22 aa, Homo sapiens
ALPDDTVIESEALPSDIAAEAR

SEQ ID NO: 25 - protein, 11 aa, Homo sapiens
AVATEAATIDR

SEQ ID NO: 26 - protein, 11 aa, Homo sapiens
ALLEEGLCAPK

SEQ ID NO: 27 - protein, 28 aa, Homo sapiens
SMGADPGPPDEKDPLGAEAAPGALGQVK

SEQ ID NO: 28 - protein, 30 aa, Homo sapiens
SMGADPGPPDEKDPLGAEAAPGALGQVKAK

SEQ ID NO: 29 - protein, 20 aa, Homo sapiens
SVPGPAVAASKENLPVLNTR

SEQ ID NO: 30 - protein, 17 aa, Homo sapiens
VVMAATEPELLDDQEAK

SEQ ID NO: 31 - protein, 18 aa, Homo sapiens
VVMAATEPELLDDQEAKR

SEQ ID NO: 32 - protein, 20 aa, Homo sapiens
MWVEPEAAAYAPPPPAKKPR

SEQ ID NO: 33 - protein, 10 aa, Homo sapiens
AIGQTLVDPK

SEQ ID NO: 34 - protein, 16 aa, Homo sapiens
AIGQTLVDPKQPLQAR

SEQ ID NO: 35 - protein, 26 aa, Homo sapiens
SLVAPDTPIQFDIISPVCEDQPGQAK

SEQ ID NO: 36 - protein, 17 aa, Homo sapiens
HYVGPAQPVPGGPPPSR

SEQ ID NO: 37 - protein, 24 aa, Homo sapiens
HYVGPAQPVPGGPPPSRGSVPVLR

SEQ ID NO: 38 - protein, 9 aa, Homo sapiens
GIGYVEDGR

SEQ ID NO: 39 - protein, 11 aa, Homo sapiens
GCPANLLSSHR

SEQ ID NO: 40 - protein, 22 aa, Homo sapiens
SLGADGTHGAGAMESAAGVLIK

SEQ ID NO: 41 - protein, 16 aa, Homo sapiens
SVGNDVDVVSDSENIK

SEQ ID NO: 42 - protein, 17 aa, Homo sapiens
SVGNDVDVVSDSENIKK

SEQ ID NO: 43 - protein, 23 aa, Homo sapiens
SSAELQSLEQQLEEAQTENFNIK

SEQ ID NO: 44 - protein, 28 aa, Homo sapiens
GLVTDSSAELQSLEQQLEEAQTENFNIK

SEQ ID NO: 45 - protein, 15 aa, Homo sapiens
LFGSDDEEESEEAKR

SEQ ID NO: 46 - protein, 14 aa, Homo sapiens
LFGSDDEEESEEAK

SEQ ID NO: 47 - protein, 26 aa, Homo sapiens
SQQPPGGSSPSEEPPPSPGEEAGLQR

SEQ ID NO: 48 - protein, 20 aa, Homo sapiens
NWDDDDDEKKEEAEVKPEVK

SEQ ID NO: 49 - protein, 14 aa, Homo sapiens
GIPEVMETQQVQEK

SEQ ID NO: 50 - protein, 22 aa, Homo sapiens
GRPPEESAHEMMEEEEEIPKPK

SEQ ID NO: 51 - protein, 14 aa, Homo sapiens
GTSELEEPLGEDPR

SEQ ID NO: 52 - protein, 23 aa, Homo sapiens
AAAATPAAPSPASLPLAPGCALR

SEQ ID NO: 53 - protein, 11 aa, Homo sapiens
GHRAPPPLVQR

SEQ ID NO: 54 - protein, 13 aa, Homo sapiens
AASQPSKGGLLER

SEQ ID NO: 55 - protein, 25 aa, Homo sapiens
GLMATTASGDITNQNSLAGGKNQGK

SEQ ID NO: 56 - protein, 21 aa, Homo sapiens
GSPVPSSPFQVPVTEGCDPSR

SEQ ID NO: 57 - protein, 23 aa, Homo sapiens
GSPVPSSPFQVPVTEGCDPSRVR

SEQ ID NO: 58 - protein, 21 aa, Homo sapiens
GVPVPGSPFPLEAVAPTKPSK

SEQ ID NO: 59 - protein, 23 aa, Homo sapiens
GVPVPGSPFPLEAVAPTKPSKVK

SEQ ID NO: 60 - protein, 39 aa, Homo sapiens
GVPVPGSPFPLEAVAPTKPSKVKAFGPGLQGGSAGSPAR

SEQ ID NO: 61 - protein, 19 aa, Homo sapiens
SAVAGAAPALVAAAAASVR

SEQ ID NO: 62 - protein, 33 aa, Homo sapiens
GLVHPPTSAAAPVTPLRPPGLGSASLHGGGPAR

SEQ ID NO: 63 - protein, 14 aa, Homo sapiens
SISSQLGPIHPPPR

SEQ ID NO: 64 - protein, 14 aa, Homo sapiens
SNSTIQEILIPASK

SEQ ID NO: 65 - protein, 16 aa, Homo sapiens
GKEFSGNPIKVSFATR

SEQ ID NO: 66 - protein, 10 aa, Homo sapiens
GKEFSGNPIK

SEQ ID NO: 67 - protein, 23 aa, Homo sapiens
GAGNLDEEQDSEGETYEDIEASK

SEQ ID NO: 68 - protein, 25 aa, Homo sapiens
GAGNLDEEQDSEGETYEDIEASKER

SEQ ID NO: 69 - protein, 16 aa, Homo sapiens
SASQAAHPQDSAFSYR

SEQ ID NO: 70 - protein, 19 aa, Homo sapiens
SASQAAHPQDSAFSYRDAK

SEQ ID NO: 71 - protein, 13 aa, Homo sapiens
SASVHDMDYVNPR

SEQ ID NO: 72 - protein, 20 aa, Homo sapiens
SSEKELEPEAAEEALENGPK

SEQ ID NO: 73 - protein, 16 aa, Homo sapiens
GLGLSYLSSHIANVER

SEQ ID NO: 74 - protein, 25 aa, Homo sapiens
TASTEETDPETSQPEPNRPSELDLR

SEQ ID NO: 75 - protein, 20 aa, Homo sapiens
GGGEVSSQGPEDSLLGTQSR

SEQ ID NO: 76 - protein, 12 aa, Homo sapiens
SAMEPTGPTQER

SEQ ID NO: 77 - protein, 30 aa, Homo sapiens
SAMEPTGPTQERYKDGVVTIGCVGFPNVGK

SEQ ID NO: 78 - protein, 18 aa, Homo sapiens
SLSMSTRPTCSESVPSAK

SEQ ID NO: 79 - protein, 13 aa, Homo sapiens
GAGPSPEEKDFLK

SEQ ID NO: 80 - protein, 9 aa, Homo sapiens
GAGPSPEEK

SEQ ID NO: 81 - protein, 18 aa, Homo sapiens
GAGPSPEEKDFLKTVEGR

SEQ ID NO: 82 - protein, 16 aa, Homo sapiens
GQIQGSVELAAPGQAK

SEQ ID NO: 83 - protein, 16 aa, Homo sapiens
GVPAEGAFTEDFQGLR

SEQ ID NO: 84 - protein, 11 aa, Homo sapiens
NIQGITKPAIR

SEQ ID NO: 85 - protein, 11 aa, Homo sapiens
FVNDISEKEQR

SEQ ID NO: 86 - protein, 8 aa, Homo sapiens
FVNDISEK

SEQ ID NO: 87 - protein, 15 aa, Homo sapiens
GGGPGQVVDDGLEHR

SEQ ID NO: 88 - protein, 22 aa, Homo sapiens
TAVITPAMLEEEEQLEAAGLER

SEQ ID NO: 89 - protein, 13 aa, Homo sapiens
LISNNEQLPMLGR

SEQ ID NO: 90 - protein, 16 aa, Homo sapiens
GSVQAAAWGPELPSHR

SEQ ID NO: 91 - protein, 36 aa, Homo sapiens
GGAGASAFEQADLNGMTPELPVAVPSGPFRHEGLSK

SEQ ID NO: 92 - protein, 15 aa, Homo sapiens
AGCLPAEEVDVLLQR

SEQ ID NO: 93 - protein, 18 aa, Homo sapiens
AVFPGPSLEPPAGSSGVK

SEQ ID NO: 94 - protein, 25 aa, Homo sapiens
GGYGGFDDYGGYNNYGYGNDGFDDR

SEQ ID NO: 95 - protein, 10 aa, Homo sapiens
GHYAMDNITR

SEQ ID NO: 96 - protein, 25 aa, Homo sapiens
ASEKPAEATAGSGGVNGGEEQGLGK

SEQ ID NO: 97 - protein, 26 aa, Homo sapiens
ASEKPAEATAGSGGVNGGEEQGLGKR

SEQ ID NO: 98 - protein, 11 aa, Homo sapiens
AVECLNYQHYK

SEQ ID NO: 99 - protein, 20 aa, Homo sapiens
AVECLNYQHYKGSDFDCELR

SEQ ID NO: 100 - protein, 31 aa, Homo sapiens
SAIDTWSPSEWQMAYEPQGGSGYDYSYAGGR

SEQ ID NO: 101 - protein, 7 aa, Homo sapiens
YSYAGGR

SEQ ID NO: 102 - protein, 19 aa, Homo sapiens
YTNPNLSGQGDPGSNPNKR

SEQ ID NO: 103 - protein, 15 aa, Homo sapiens
SLSPETYGNFDSQSR

SEQ ID NO: 104 - protein, 24 aa, Homo sapiens
AGGEPDSLGQQPTDTPYEWDLDKK

SEQ ID NO: 105 - protein, 29 aa, Homo sapiens
AGGEPDSLGQQPTDTPYEWDLDKKAWFPK

SEQ ID NO: 106 - protein, 15 aa, Homo sapiens
GASSSTANVEDVHAR

SEQ ID NO: 107 - protein, 11 aa, Homo sapiens
GLPEEQPQTTK

SEQ ID NO: 108 - protein, 8 aa, Homo sapiens
MNASPLVR

SEQ ID NO: 109 - protein, 11 aa, Homo sapiens
SAVAISGADSR

SEQ ID NO: 110 - protein, 18 aa, Homo sapiens
SVLAVMPPDIAAEAQALR

SEQ ID NO: 111 - protein, 16 aa, Homo sapiens
ALSSDFTCGSPTAAGK

SEQ ID NO: 112 - protein, 17 aa, Homo sapiens
ALSSDFTCGSPTAAGKK

SEQ ID NO: 113 - protein, 14 aa, Homo sapiens
SSSAPSKEQLEQEK

SEQ ID NO: 114 - protein, 26 aa, Homo sapiens
SSSAPSKEQLEQEKQLLLSFKPVMQK

SEQ ID NO: 115 - protein, 16 aa, Homo sapiens
SLKEALTYDGALLGDR

SEQ ID NO: 116 - protein, 18 aa, Homo sapiens
GLTPQLEEDEELQGHLGR

SEQ ID NO: 117 - protein, 19 aa, Homo sapiens
GLTPQLEEDEELQGHLGRR

SEQ ID NO: 118 - protein, 21 aa, Homo sapiens
GSGIYDPCEKEATDAIGHLDR

SEQ ID NO: 119 - protein, 9 aa, Homo sapiens
CFLEEIMTK

SEQ ID NO: 120 - protein, 23 aa, Homo sapiens
AVTPGPQPTLEQLEEGGPRPLER

SEQ ID NO: 121 - protein, 24 aa, Homo sapiens
AVTPGPQPTLEQLEEGGPRPLERR

SEQ ID NO: 122 - protein, 17 aa, Homo sapiens
GLGVARPHYGSVLDNER

SEQ ID NO: 123 - protein, 26 aa, Homo sapiens
GLGVARPHYGSVLDNERLTAEEMDER

SEQ ID NO: 124 - protein, 12 aa, Homo sapiens
GVIVPLEQEYEK

SEQ ID NO: 125 - protein, 13 aa, Homo sapiens
GVIVPLEQEYEKK

SEQ ID NO: 126 - protein, 15 aa, Homo sapiens
GAEVPNPDSVTDDLK

SEQ ID NO: 127 - protein, 19 aa, Homo sapiens
GAEVPNPDSVTDDLKVSEK

SEQ ID NO: 128 - protein, 10 aa, Homo sapiens
AVQMANEELR

SEQ ID NO: 129 - protein, 12 aa, Homo sapiens
HVTSDAVELANR

SEQ ID NO: 130 - protein, 22 aa, Homo sapiens
GGLGSCQALEDHSALAETQEDR

SEQ ID NO: 131 - protein, 21 aa, Homo sapiens
GSPAGAEDSLEEEASSEGEPR

SEQ ID NO: 132 - protein, 13 aa, Homo sapiens
SIGAVLNSKDEQR

SEQ ID NO: 133 - protein, 19 aa, Homo sapiens
SIGAVLNSKDEQREIAETR

SEQ ID NO: 134 - protein, 9 aa, Homo sapiens
SIGAVLNSK

SEQ ID NO: 135 - protein, 17 aa, Homo sapiens
AGAEPITADSDPAYSSK

SEQ ID NO: 136 - protein, 21 aa, Homo sapiens
ATVGGPAPTPLLPPSATASVK

SEQ ID NO: 137 - protein, 22 aa, Homo sapiens
GADCIMLSGETAKGDYPLEAVR

SEQ ID NO: 138 - protein, 13 aa, Homo sapiens
GADCIMLSGETAK

SEQ ID NO: 139 - protein, 9 aa, Homo sapiens
GWKEPAFSK

SEQ ID NO: 140 - protein, 14 aa, Homo sapiens
GWKEPAFSKEDNPR

SEQ ID NO: 141 - protein, 21 aa, Homo sapiens
AALEVSPGVIANPFAAGIGHR

SEQ ID NO: 142 - protein, 13 aa, Homo sapiens
AINWPTPGEIAHK

SEQ ID NO: 143 - protein, 13 aa, Homo sapiens
FSQLLNCPEFVPR

SEQ ID NO: 144 - protein, 19 aa, Homo sapiens
GLNQTTIPVSPPSTTKPSR

SEQ ID NO: 145 - protein, 20 aa, Homo sapiens
LAKEPCLHPLEPDEVEYEPR

SEQ ID NO: 146 - protein, 18 aa, Homo sapiens
GVLDINHEQENTPSTSGK

SEQ ID NO: 147 - protein, 19 aa, Homo sapiens
GVLDINHEQENTPSTSGKR

SEQ ID NO: 148 - protein, 15 aa, Homo sapiens
SAQASDMGGESPGSR

SEQ ID NO: 149 - protein, 17 aa, Homo sapiens
AVSGQLPDPTTNPSAGK

SEQ ID NO: 150 - protein, 29 aa, Homo sapiens
AVSGQLPDPTTNPSAGKDGPSLLVVEQVR

SEQ ID NO: 151 - protein, 12 aa, Homo sapiens
SVVSPLPVTTVK

SEQ ID NO: 152 - protein, 19 aa, Homo sapiens
SAGLLSDEDCMSVPGKTHR

SEQ ID NO: 153 - protein, 11 aa, Homo sapiens
GFDVASVQQQR

SEQ ID NO: 154 - protein, 31 aa, Homo sapiens
AAVLTSPPAPAPPVTPSKPMAGTTDREEATR

SEQ ID NO: 155 - protein, 20 aa, Homo sapiens
GYMGVNQAPEKLDKQCEMMK

SEQ ID NO: 156 - protein, 14 aa, Homo sapiens
GYMGVNQAPEKLDK

SEQ ID NO: 157 - protein, 11 aa, Homo sapiens
GYMGVNQAPEK

SEQ ID NO: 158 - protein, 12 aa, Homo sapiens
SVIGVSPAVMIR

SEQ ID NO: 159 - protein, 11 aa, Homo sapiens
GFAEEAPSTSR

SEQ ID NO: 160 - protein, 28 aa, Homo sapiens
GFAEEAPSTSRGPGGSQGSQGPSPQGAR

SEQ ID NO: 161 - protein, 23 aa, Homo sapiens
SVVAAVQEGAAELEGGPYSPLGK

SEQ ID NO: 162 - protein, 26 aa, Homo sapiens
SVVAAVQEGAAELEGGPYSPLGKDYR

SEQ ID NO: 163 - protein, 27 aa, Homo sapiens
SVVAAVQEGAAELEGGPYSPLGKDYRK

SEQ ID NO: 164 - protein, 17 aa, Homo sapiens
GQSDENKDDYTIPDEYR

SEQ ID NO: 165 - protein, 12 aa, Homo sapiens
LANLGDVASDGK

SEQ ID NO: 166 - protein, 13 aa, Homo sapiens
LANLGDVASDGKK

SEQ ID NO: 167 - protein, 20 aa, Homo sapiens
SFDDRGPSLNPVLDYDHGSR

SEQ ID NO: 168 - protein, 15 aa, Homo sapiens
YYTTTPALVFGKPVR

SEQ ID NO: 169 - protein, 18 aa, Homo sapiens
LANLGDVASDGKKEPSDK

SEQ ID NO: 170 - protein, 21 aa, Homo sapiens
SYDPYDFSDTEEEMPQVHTPK

SEQ ID NO: 171 - protein, 17 aa, Homo sapiens
GAAAEDIVASEQSLGQK

SEQ ID NO: 172 - protein, 15 aa, Homo sapiens
SFGGDAQADEGQARK

SEQ ID NO: 173 - protein, 14 aa, Homo sapiens
SFGGDAQADEGQAR

SEQ ID NO: 174 - protein, 11 aa, Homo sapiens
GYETEGIRGLR

SEQ ID NO: 175 - protein, 8 aa, Homo sapiens
GYETEGIR

SEQ ID NO: 176 - protein, 23 aa, Homo sapiens
ALSPEQPASHESQGSVPSPLEAR

SEQ ID NO: 177 - protein, 11 aa, Homo sapiens
ALYVACQGQPK

SEQ ID NO: 178 - protein, 19 aa, Homo sapiens
GSAVNGTSSAETNLEALQK

SEQ ID NO: 179 - protein, 20 aa, Homo sapiens
GSAVNGTSSAETNLEALQKK

SEQ ID NO: 180 - protein, 12 aa, Homo sapiens
AALLAPEEIKEK

SEQ ID NO: 181 - protein, 10 aa, Homo sapiens
AALLAPEEIK

SEQ ID NO: 182 - protein, 14 aa, Homo sapiens
AIEPAPPSQGAEAK

SEQ ID NO: 183 - protein, 9 aa, Homo sapiens
TTAAQQELR

SEQ ID NO: 184 - protein, 9 aa, Homo sapiens
STATQQELR

SEQ ID NO: 185 - protein, 9 aa, Homo sapiens
STAAQQELR

SEQ ID NO: 186 - protein, 15 aa, Homo sapiens
SLTSDKASVPIVLEK

SEQ ID NO: 187 - protein, 25 aa, Homo sapiens
KIEDVPAPSTSADKVESLDVDSEAK

SEQ ID NO: 188 - protein, 15 aa, Homo sapiens
SASPADDSFVDPGER

SEQ ID NO: 189 - protein, 12 aa, Homo sapiens
AAASAPQMDVSK

SEQ ID NO: 190 - protein, 17 aa, Homo sapiens
GTSLVGVTQSFAASVLR

SEQ ID NO: 191 - protein, 17 aa, Homo sapiens
AVAEQGHLPPPSAPAGR

SEQ ID NO: 192 - protein, 31 aa, Homo sapiens
GGLQINVDEEPFVLPPAGEMEQDAQAPDLQR

SEQ ID NO: 193 - protein, 35 aa, Homo sapiens
GGLQINVDEEPFVLPPAGEMEQDAQAPDLQRVHKR

SEQ ID NO: 194 - protein, 9 aa, Homo sapiens
GLIPGVEPR

SEQ ID NO: 195 - protein, 15 aa, Homo sapiens
GLVETPTGYIESLPR

SEQ ID NO: 196 - protein, 19 aa, Homo sapiens
GLVETPTGYIESLPRVVKR

SEQ ID NO: 197 - protein, 11 aa, Homo sapiens
GVPSDSVEAAK

SEQ ID NO: 198 - protein, 18 aa, Homo sapiens
GVPSDSVEAAKNASNTEK

SEQ ID NO: 199 - protein, 28 aa, Homo sapiens
GVPSDSVEAAKNASNTEKLTDQVMQNPR

SEQ ID NO: 200 - protein, 15 aa, Homo sapiens
NVPHTPSSYIETLPK

SEQ ID NO: 201 - protein, 25 aa, Homo sapiens
GQKVEVPQPLSWYPEELAWHTNLSR

SEQ ID NO: 202 - protein, 26 aa, Homo sapiens
GQKVEVPQPLSWYPEELAWHTNLSRK

SEQ ID NO: 203 - protein, 14 aa, Homo sapiens
GGFEGDHQLLCDIR

SEQ ID NO: 204 - protein, 18 aa, Homo sapiens
FTQESEPSYISDVGPPGR

SEQ ID NO: 205 - protein, 15 aa, Homo sapiens
AINQGMDEELERDEK

SEQ ID NO: 206 - protein, 11 aa, Homo sapiens
AAVADPDEFER

SEQ ID NO: 207 - protein, 12 aa, Homo sapiens
GAIEDPELEAIK

SEQ ID NO: 208 - protein, 14 aa, Homo sapiens
GAIEDPELEAIKAR

SEQ ID NO: 209 - protein, 17 aa, Homo sapiens
AYSIQGQHTISPLDLAK

SEQ ID NO: 210 - protein, 22 aa, Homo sapiens
ASTQTTHELTIPNNLIGCIIGR

SEQ ID NO: 211 - protein, 15 aa, Homo sapiens
GRGEPAMESSQIVSR

SEQ ID NO: 212 - protein, 12 aa, Homo sapiens
AYTAPALPSSIR

SEQ ID NO: 213 - protein, 8 aa, Homo sapiens
CVGPEVEK

SEQ ID NO: 214 - protein, 25 aa, Homo sapiens
CVGPEVEKACANPAAGSVILLENLR

SEQ ID NO: 215 - protein, 19 aa, Homo sapiens
SITSQESKEPVFIAAGDIR

SEQ ID NO: 216 - protein, 20 aa, Homo sapiens
SITSQESKEPVFIAAGDIRR

SEQ ID NO: 217 - protein, 17 aa, Homo sapiens
GHPASSPLLPVSLLGPK

SEQ ID NO: 218 - protein, 14 aa, Homo sapiens
ADIKPPENVLFVCK

SEQ ID NO: 219 - protein, 21 aa, Homo sapiens
GDPSDRMEVQEQEEDISSLIR

SEQ ID NO: 220 - protein, 19 aa, Homo sapiens
VAKPSEEEQKELDEITAKR

SEQ ID NO: 221 - protein, 8 aa, Homo sapiens
GIGEEVLK

SEQ ID NO: 222 - protein, 17 aa, Homo sapiens
GIGEEVLKMSTEEIIQR

SEQ ID NO: 223 - protein, 9 aa, Homo sapiens
GFDQNVNVK

SEQ ID NO: 224 - protein, 19 aa, Homo sapiens
SIGSSRLEGGSGGDSEVQR

SEQ ID NO: 225 - protein, 11 aa, Homo sapiens
SFMDPASALYR

SEQ ID NO: 226 - protein, 8 aa, Homo sapiens
YSATVDQR

SEQ ID NO: 227 - protein, 9 aa, Homo sapiens
SVITQVLNK

SEQ ID NO: 228 - protein, 12 aa, Homo sapiens
GVDQQLLDDFHR

SEQ ID NO: 229 - protein, 24 aa, Homo sapiens
GGPNTGGMGAYCPAPQVSNDLLLK

SEQ ID NO: 230 - protein, 17 aa, Homo sapiens
GIIAPGYEEEALTILSK

SEQ ID NO: 231 - protein, 25 aa, Homo sapiens
GQQSAPQADEPPLPPPPPPPGELAR

SEQ ID NO: 232 - protein, 20 aa, Homo sapiens
GTLNLDSDEGEEPSPEALVR

SEQ ID NO: 233 - protein, 35 aa, Homo sapiens
SVDPVEPMPTMTDQTTLVPNEEEAFALEPIDITVK

SEQ ID NO: 234 - protein, 38 aa, Homo sapiens
SVDPVEPMPTMTDQTTLVPNEEEAFALEPIDITVKETK

SEQ ID NO: 235 - protein, 11 aa, Homo sapiens
VAQQFSLNQSR

SEQ ID NO: 236 - protein, 15 aa, Homo sapiens
SDKGEFGGFGSVTGK

SEQ ID NO: 237 - protein, 12 aa, Homo sapiens
GVEEEVFEQEAK

SEQ ID NO: 238 - protein, 18 aa, Homo sapiens
GYNPEAPSITNTSRPMYR

SEQ ID NO: 239 - protein, 15 aa, Homo sapiens
ASSASSFLDSDELER

SEQ ID NO: 240 - protein, 25 aa, Homo sapiens
ASSASSFLDSDELERTGIDLGTTGR

SEQ ID NO: 241 - protein, 13 aa, Homo sapiens
SVEQDGDEPGPQR

SEQ ID NO: 242 - protein, 28 aa, Homo sapiens
GGSAHGDDDDDGPHFEPVVPLPDKIEVK

SEQ ID NO: 243 - protein, 20 aa, Homo sapiens
GTGGQSIYGDKFEDENFDVK

SEQ ID NO: 244 - protein, 9 aa, Homo sapiens
SIDSFETQR

SEQ ID NO: 245 - protein, 19 aa, Homo sapiens
GVNKDYEETELISTTANYR

SEQ ID NO: 246 - protein, 7 aa, Homo sapiens
YVPSTTK

SEQ ID NO: 247 - protein, 10 aa, Homo sapiens
YVPSTTKTPR

SEQ ID NO: 248 - protein, 11 aa, Homo sapiens
GYYEAEFGQER

SEQ ID NO: 249 - protein, 15 aa, Homo sapiens
SFTSDPEQIGSNVTR

SEQ ID NO: 250 - protein, 19 aa, Homo sapiens
SNIAPADPDTAIVHPVPIR

SEQ ID NO: 251 - protein, 21 aa, Homo sapiens
GYSSSDSFTSDPEQIGSNVTR

SEQ ID NO: 252 - protein, 9 aa, Homo sapiens
GSSLEALLR

SEQ ID NO: 253 - protein, 8 aa, Homo sapiens
GMAGNEDR

SEQ ID NO: 254 - protein, 19 aa, Homo sapiens
GMAGNEDRGGIQELIGLIK

SEQ ID NO: 255 - protein, 32 aa, Homo sapiens
SFSSMTSDSDTTSPTGQQPSDAFPEDSSKVPR

SEQ ID NO: 256 - protein, 16 aa, Homo sapiens
GCLCPCSLGLGGVGMR

SEQ ID NO: 257 - protein, 27 aa, Homo sapiens
SIEAAEGEQEPEAEALGGTNSEPGTPR

SEQ ID NO: 258 - protein, 14 aa, Homo sapiens
SLENIPEKWTPEVK

SEQ ID NO: 259 - protein, 9 aa, Homo sapiens
GTEIAVSPR

SEQ ID NO: 260 - protein, 16 aa, Homo sapiens
GQPGAFTCYLDAGLAR

SEQ ID NO: 261 - protein, 13 aa, Homo sapiens
GVREEDLAPFSLR

SEQ ID NO: 262 - protein, 12 aa, Homo sapiens
SVKPGAHLTVKK

SEQ ID NO: 263 - protein, 17 aa, Homo sapiens
SFNGHPPQGCASTPVAR

SEQ ID NO: 264 - protein, 10 aa, Homo sapiens
SMSAALQDER

SEQ ID NO: 265 - protein, 30 aa, Homo sapiens
STAGTTKQPSKEEEEEEEEEQLNQTLAEMK

SEQ ID NO: 266 - protein, 27 aa, Homo sapiens
GNKMEEEEGAKGEDEEMADPMEDVIIR

SEQ ID NO: 267 - protein, 8 aa, Homo sapiens
SFVQQTFR

SEQ ID NO: 268 - protein, 13 aa, Homo sapiens
GYGEDLMGDEEDR

SEQ ID NO: 269 - protein, 15 aa, Homo sapiens
GYGEDLMGDEEDRAR

SEQ ID NO: 270 - protein, 12 aa, Homo sapiens
TYLTHDSPSVRK

SEQ ID NO: 271 - protein, 11 aa, Homo sapiens
TYLTHDSPSVR

SEQ ID NO: 272 - protein, 10 aa, Homo sapiens
AIDFSDNEIR

SEQ ID NO: 273 - protein, 22 aa, Homo sapiens
STAPSSELGKDDLEELAAAAQK

SEQ ID NO: 274 - protein, 9 aa, Homo sapiens
AYGEDDFSR

SEQ ID NO: 275 - protein, 19 aa, Homo sapiens
SYSSAASYTDSSDDEVSPR

SEQ ID NO: 276 - protein, 21 aa, Homo sapiens
SYSSAASYTDSSDDEVSPREK

SEQ ID NO: 277 - protein, 11 aa, Homo sapiens
VYCEVCEFLVK

SEQ ID NO: 278 - protein, 15 aa, Homo sapiens
VYCEVCEFLVKEVTK

SEQ ID NO: 279 - protein, 10 aa, Homo sapiens
YSLDSPGPEK

SEQ ID NO: 280 - protein, 14 aa, Homo sapiens
SVSRPQLESLSGTK

SEQ ID NO: 281 - protein, 16 aa, Homo sapiens
STGYYDQEIYGGSDSR

SEQ ID NO: 282 - protein, 14 aa, Homo sapiens
GSETPQLFTVLPEK

SEQ ID NO: 283 - protein, 15 aa, Homo sapiens
GSETPQLFTVLPEKR

SEQ ID NO: 284 - protein, 28 aa, Homo sapiens
GLPGEAAEDDLAGAPALSQASSGTCFPR

SEQ ID NO: 285 - protein, 20 aa, Homo sapiens
SFCSDQNESEVEPSVNADLK

SEQ ID NO: 286 - protein, 14 aa, Homo sapiens
GGSPPGPGDLAEER

SEQ ID NO: 287 - protein, 19 aa, Homo sapiens
AAAPADAPSGLEAELEHLR

SEQ ID NO: 288 - protein, 23 aa, Homo sapiens
GEVTVAEQKPGEIAEELASSYER

SEQ ID NO: 289 - protein, 23 aa, Homo sapiens
GVADVSIEDSVISLSGDHCIIGR

SEQ ID NO: 290 - protein, 14 aa, Homo sapiens
SVISLSGDHCIIGR

SEQ ID NO: 291 - protein, 19 aa, Homo sapiens
SFLKFDSEPSAVALELPTR

SEQ ID NO: 292 - protein, 19 aa, Homo sapiens
SAGIQLHPGENADSPADIR

SEQ ID NO: 293 - protein, 12 aa, Homo sapiens
SFASTQPTHSWK

SEQ ID NO: 294 - protein, 10 aa, Homo sapiens
SYIEVLDGSR

SEQ ID NO: 295 - protein, 26 aa, Homo sapiens
GAPLEDVDGIPIDATPIDDLDGVPIK

SEQ ID NO: 296 - protein, 23 aa, Homo sapiens
GVPIKSLDDDLDGVPLDATEDSK

SEQ ID NO: 297 - protein, 24 aa, Homo sapiens
GVPIKSLDDDLDGVPLDATEDSKK

SEQ ID NO: 298 - protein, 11 aa, Homo sapiens
GVPLDATEDSK

SEQ ID NO: 299 - protein, 12 aa, Homo sapiens
GVPLDATEDSKK

SEQ ID NO: 300 - protein, 18 aa, Homo sapiens
GVPLDATEDSKKNEPIFK

SEQ ID NO: 301 - protein, 22 aa, Homo sapiens
GFPAGEGEEAGRPGAEDEEMSR

SEQ ID NO: 302 - protein, 22 aa, Homo sapiens
GFPAGEGEEAGRPGAEDEEMSR

SEQ ID NO: 303 - protein, 9 aa, Homo sapiens
SVVSLESQK

SEQ ID NO: 304 - protein, 17 aa, Homo sapiens
SVVSLESQKTPADPKLK

SEQ ID NO: 305 - protein, 20 aa, Homo sapiens
AHQTETSSSQVKDNKPLVER

SEQ ID NO: 306 - protein, 12 aa, Homo sapiens
AHQTETSSSQVK

SEQ ID NO: 307 - protein, 9 aa, Homo sapiens
SNSLLGQSR

SEQ ID NO: 308 - protein, 14 aa, Homo sapiens
VLNPMPACFYTVSR

SEQ ID NO: 309 - protein, 33 aa, Homo sapiens
GANTMIEHDDTLPSQLGTMVINAEDEEEEGTMK

SEQ ID NO: 310 - protein, 34 aa, Homo sapiens
GANTMIEHDDTLPSQLGTMVINAEDEEEEGTMKR

SEQ ID NO: 311 - protein, 35 aa, Homo sapiens
GANTMIEHDDTLPSQLGTMVINAEDEEEEGTMKRR

SEQ ID NO: 312 - protein, 10 aa, Homo sapiens
SLTYDIANNK

SEQ ID NO: 313 - protein, 14 aa, Homo sapiens
SLTYDIANNKDALR

SEQ ID NO: 314 - protein, 15 aa, Homo sapiens
SLTYDIANNKDALRK

SEQ ID NO: 315 - protein, 15 aa, Homo sapiens
SIEANVESSEVHVER

SEQ ID NO: 316 - protein, 22 aa, Homo sapiens
SIEANVENAEVHVQQANQQLSR

SEQ ID NO: 317 - protein, 16 aa, Homo sapiens
ALIDEDPQAALEELTK

SEQ ID NO: 318 - protein, 17 aa, Homo sapiens
SAEEGSLAAAAELAAQK

SEQ ID NO: 319 - protein, 18 aa, Homo sapiens
SAEEGSLAAAAELAAQKR

SEQ ID NO: 320 - protein, 13 aa, Homo sapiens
FVDPWTVQTSSAK

SEQ ID NO: 321 - protein, 20 aa, Homo sapiens
AFQEREEGHAGPDDNEEVMR

SEQ ID NO: 322 - protein, 17 aa, Homo sapiens
GHATDEEKLASTSCGQK

SEQ ID NO: 323 - protein, 8 aa, Homo sapiens
GHATDEEK

SEQ ID NO: 324 - protein, 15 aa, Homo sapiens
GIPEETDGDADVDLK

SEQ ID NO: 325 - protein, 10 aa, Homo sapiens
AVMPDVVQTR

SEQ ID NO: 326 - protein, 17 aa, Homo sapiens
GKQEAKPQQAAGMLSPK

SEQ ID NO: 327 - protein, 15 aa, Homo sapiens
GLSVSQAPAILPVSK

SEQ ID NO: 328 - protein, 20 aa, Homo sapiens
AFIAAGESSAPTPPRPALPR

SEQ ID NO: 329 - protein, 35 aa, Homo sapiens
ANQCCTSCEDNAPATSYCVECSEPLCETCVEAHQR

SEQ ID NO: 330 - protein, 18 aa, Homo sapiens
STFSLDQPGGTLDLTLIR

SEQ ID NO: 331 - protein, 17 aa, Homo sapiens
SVVPFDAASEVPVEEQR

SEQ ID NO: 332 - protein, 17 aa, Homo sapiens
SVNLAEPMEQNPPQQQR

SEQ ID NO: 333 - protein, 15 aa, Homo sapiens
SMRGEAPGAETPSLR

SEQ ID NO: 334 - protein, 17 aa, Homo sapiens
SIKPEETEQEVAADETR

SEQ ID NO: 335 - protein, 17 aa, Homo sapiens
SILKSELGNQSPSTSSR

SEQ ID NO: 336 - protein, 10 aa, Homo sapiens
MLSPIHTPQR

SEQ ID NO: 337 - protein, 31 aa, Homo sapiens
GCSTPSREEGGCSLASTPATTLHLLQLSGQR

SEQ ID NO: 338 - protein, 10 aa, Homo sapiens
SSQPSLPLVR

SEQ ID NO: 339 - protein, 34 aa, Homo sapiens
SAGTSPTAVLAAGEEVGAGGGPGGGRPGAGTPLR

SEQ ID NO: 340 - protein, 47 aa, Homo sapiens
SAGTSPTAVLAAGEEVGAGGGPGGGRPGAGTPLRQTLWPLSIHDPTR

SEQ ID NO: 341 - protein, 17 aa, Homo sapiens
SFDEDLARPSGLLAQER

SEQ ID NO: 342 - protein, 20 aa, Homo sapiens
GSNVTVTPGPGEQTVDVEPR

SEQ ID NO: 343 - protein, 24 aa, Homo sapiens
GVPSTSPMEVLDRLIQQGADAHSK

SEQ ID NO: 344 - protein, 13 aa, Homo sapiens
GVPSTSPMEVLDR

SEQ ID NO: 345 - protein, 10 aa, Homo sapiens
AVDDMEEGLK

SEQ ID NO: 346 - protein, 14 aa, Homo sapiens
AVDDMEEGLKVLMK

SEQ ID NO: 347 - protein, 12 aa, Homo sapiens
STGRPLVILPQR

SEQ ID NO: 348 - protein, 13 aa, Homo sapiens
STGRPLVILPQRK

SEQ ID NO: 349 - protein, 18 aa, Homo sapiens
GLAVTPTPVPVVGSQMTR

SEQ ID NO: 350 - protein, 19 aa, Homo sapiens
GVPQVVEYSEISPETAQLR

SEQ ID NO: 351 - protein, 16 aa, Homo sapiens
ALECLPEDKEVLTEDK

SEQ ID NO: 352 - protein, 29 aa, Homo sapiens
GSASPVEMQDEGAEEPHEAGEQLPPFLLK

SEQ ID NO: 353 - protein, 32 aa, Homo sapiens
GSASPVEMQDEGAEEPHEAGEQLPPFLLKEGR

SEQ ID NO: 354 - protein, 35 aa, Homo sapiens
GSASPVEMQDEGAEEPHEAGEQLPPFLLKEGRDDR

SEQ ID NO: 355 - protein, 20 aa, Homo sapiens
SFIDNSEAYDELVPASLTTK

SEQ ID NO: 356 - protein, 14 aa, Homo sapiens
GRPDEVVAEEAWQR

SEQ ID NO: 357 - protein, 14 aa, Homo sapiens
GSLASNPYSGDLTK

SEQ ID NO: 358 - protein, 16 aa, Homo sapiens
GHNTAEEDMEDDTSWR

SEQ ID NO: 359 - protein, 11 aa, Homo sapiens
SVAGEHSVSGR

SEQ ID NO: 360 - protein, 11 aa, Homo sapiens
GFRDFQTETIR

SEQ ID NO: 361 - protein, 17 aa, Homo sapiens
GFRDFQTETIRQEQELR

SEQ ID NO: 362 - protein, 18 aa, Homo sapiens
SRPADEDMWDETELGLYK

SEQ ID NO: 363 - protein, 26 aa, Homo sapiens
SRPADEDMWDETELGLYKVNEYVDAR

SEQ ID NO: 364 - protein, 9 aa, Homo sapiens
SLTTIPELK

SEQ ID NO: 365 - protein, 13 aa, Homo sapiens
SLTTIPELKDHLR

SEQ ID NO: 366 - protein, 7 aa, Homo sapiens
AINTEFK

SEQ ID NO: 367 - protein, 10 aa, Homo sapiens
AINTEFKNTR

SEQ ID NO: 368 - protein, 11 aa, Homo sapiens
ALKGTNESLER

SEQ ID NO: 369 - protein, 12 aa, Homo sapiens
FSLADAINTEFK

SEQ ID NO: 370 - protein, 15 aa, Homo sapiens
FSLADAINTEFKNTR

SEQ ID NO: 371 - protein, 13 aa, Homo sapiens
VDVSKPDLTAALR

SEQ ID NO: 372 - protein, 16 aa, Homo sapiens
VDVSKPDLTAALRDVR

SEQ ID NO: 373 - protein, 11 aa, Homo sapiens
VSKPDLTAALR

SEQ ID NO: 374 - protein, 14 aa, Homo sapiens
VSKPDLTAALRDVR

SEQ ID NO: 375 - protein, 11 aa, Homo sapiens
SVGTYLPGASR

SEQ ID NO: 376 - protein, 15 aa, Homo sapiens
SLCGSRNENESEAAR

SEQ ID NO: 377 - protein, 16 aa, Homo sapiens
SLCGSRNENESEAARR

SEQ ID NO: 378 - protein, 19 aa, Homo sapiens
GIGGLGIGEGAPEIVTGSR

SEQ ID NO: 379 - protein, 14 aa, Homo sapiens
GIPNDSSDSEMEDK

SEQ ID NO: 380 - protein, 26 aa, Homo sapiens
GNGVGQSQAGSGSTPSEPHPVLEKLR

SEQ ID NO: 381 - protein, 12 aa, Homo sapiens
GQSAFANETLNK

SEQ ID NO: 382 - protein, 27 aa, Homo sapiens
GQSAFANETLNKAPGMNTIDQGMAALK

SEQ ID NO: 383 - protein, 39 aa, Homo sapiens
GQSAFANETLNKAPGMNTIDQGMAALKLGSTEVASNVPK

SEQ ID NO: 384 - protein, 15 aa, Homo sapiens
GSISPVSSECSVVER

SEQ ID NO: 385 - protein, 35 aa, Homo sapiens
GVPSPPGYMSDQEEDMCFEGMKPVNQTAASNKGLR

SEQ ID NO: 386 - protein, 10 aa, Homo sapiens
GVATDITSTR

SEQ ID NO: 387 - protein, 12 aa, Homo sapiens
SLSDVTSTTSSR

SEQ ID NO: 388 - protein, 17 aa, Homo sapiens
TVLEPYADPYYDYEIER

SEQ ID NO: 389 - protein, 12 aa, Homo sapiens
GETEVGEIQQNK

SEQ ID NO: 390 - protein, 22 aa, Homo sapiens
AADCEGVPEDDLPTDQTVLPGR

SEQ ID NO: 391 - protein, 8 aa, Homo sapiens
GTCPEVIK

SEQ ID NO: 392 - protein, 13 aa, Homo sapiens
GTCPEVIKVYIFK

SEQ ID NO: 393 - protein, 30 aa, Homo sapiens
STESIPVSDEDSDAMVDDPNDEDFVPFRPR

SEQ ID NO: 394 - protein, 11 aa, Homo sapiens
SFGSPLGLDKR

SEQ ID NO: 395 - protein, 12 aa, Homo sapiens
SFGSPLGLDKRK

SEQ ID NO: 396 - protein, 20 aa, Homo sapiens
AIDDQKCDILVQEELLASPK

SEQ ID NO: 397 - protein, 21 aa, Homo sapiens
AIDDQKCDILVQEELLASPKK

SEQ ID NO: 398 - protein, 16 aa, Homo sapiens
SILIIPTPDEEEKILR

SEQ ID NO: 399 - protein, 13 aa, Homo sapiens
SILIIPTPDEEEK

SEQ ID NO: 400 - protein, 11 aa, Homo sapiens
SLSSLLDDMTK

SEQ ID NO: 401 - protein, 18 aa, Homo sapiens
SLSSLLDDMTKNDPFKAR

SEQ ID NO: 402 - protein, 14 aa, Homo sapiens
SYAAASAPQMDVSK

SEQ ID NO: 403 - protein, 7 aa, Homo sapiens
LVDAAAS

SEQ ID NO: 404 - protein, 12 aa, Homo sapiens
SYGLSEQENNEK

SEQ ID NO: 405 - protein, 8 aa, Homo sapiens
EIIDGLSE

SEQ ID NO: 406 - protein, 17 aa, Homo sapiens
SYGTAEETEEREQATPR

SEQ ID NO: 407 - protein, 8 aa, Homo sapiens
DKIDGTAE

SEQ ID NO: 408 - protein, 16 aa, Homo sapiens
SYGDVEIPPNKAVVLR

SEQ ID NO: 409 - protein, 8 aa, Homo sapiens
MEVDGDVE

SEQ ID NO: 410 - protein, 12 aa, Homo sapiens
SYAVMPDVVQTR

SEQ ID NO: 411 - protein, 8 aa, Homo sapiens
SLIDAVMP

SEQ ID NO: 412 - protein, 17 aa, Homo sapiens
SYGGGPGQVVDDGLEHR

SEQ ID NO: 413 - protein, 8 aa, Homo sapiens
LETDGGGP

SEQ ID NO: 414 - protein, 14 aa, Homo sapiens
SYSIQEPVVLFHSR

SEQ ID NO: 415 - protein, 8 aa, Homo sapiens
STTDSIQE

SEQ ID NO: 416 - protein, 17 aa, Homo sapiens
SYSDKGEFGGFGSVTGK

SEQ ID NO: 417 - protein, 8 aa, Homo sapiens
SHCDSDKG

SEQ ID NO: 418 - protein, 17 aa, Homo sapiens
AANSNGPFQPVVLLHIR

SEQ ID NO: 419 - protein, 8 aa, Homo sapiens
AGSRMVVD

SEQ ID NO: 420 - protein, 8 aa, Artificial sequence
  Synthetic polypeptide
ENLYFQSY

SEQ ID NO: 421 - protein, 8 aa, Artificial sequence
  Synthetic polypeptide
ENLYFQSK

SEQ ID NO: 422 - protein, 8 aa, Artificial sequence
  Synthetic polypeptide
ENLYFQSA

SEQ ID NO: 423 - protein, 4 aa, Artificial sequence
  Synthetic polypeptide
AAPY

SEQ ID NO: 424 - protein, 4 aa, Artificial sequence
  Synthetic polypeptide
AAPK

SEQ ID NO: 425 - protein, 4 aa, Artificial sequence
  Synthetic polypeptide
AAPA

SEQ ID NO: 426 - protein, 7 aa, Artificial sequence
  Synthetic polypeptide
EXLFQGP

SEQ ID NO: 427 - protein, 5 aa, Artificial sequence
  Synthetic polypeptide
DDDDK

SEQ ID NO: 428 - protein, 4 aa, Artificial sequence
  Synthetic polypeptide
LVPR

SEQ ID NO: 429 - protein, 4 aa, Artificial sequence
  Synthetic polypeptide
IEPD

SEQ ID NO: 430 - protein, 4 aa, Artificial sequence
  Synthetic polypeptide
DEVD

SEQ ID NO: 431 - protein, 21 aa, Artificial sequence
  Synthetic polypeptide
SYGSAVNGTSSAETNLEALQK

SEQ ID NO: 432 - protein, 50 aa, Homo sapiens
MPKKKPTPIQLNPAPDGSAVNGTSSAETNLEALQKKLEELELDEQQRKRL

SEQ ID NO: 433 - protein, 8 aa, Homo sapiens
PAPDGSAV

SEQ ID NO: 434 - protein, 10 aa, Artificial sequence
  Synthetic polypeptide
GTENLYFQSY

SEQ ID NO: 435 - protein, 10 aa, Artificial sequence
  Synthetic polypeptide
KGGTENLYFQ

SEQ ID NO: 436 - protein, 7 aa, Artificial Sequence
  Synthetic polypeptide
EXXYXQX

SEQ ID NO: 437 - protein, 4 aa, Artificial Sequence
  Synthetic polypeptide
RXXR

SEQ ID NO: 438 - protein, 4 aa, Artificial Sequence
  Synthetic polypeptide
DXXD

SEQ ID NO: 439 - protein, 11 aa, Homo sapiens
AITELEDAFSR

SEQ ID NO: 440 - protein, 20 aa, Homo sapiens
AAAVAVPLAGGQEGSPGGGR

SEQ ID NO: 441 - protein, 8 aa, Homo sapiens
STFYLGER

SEQ ID NO: 442 - protein, 17 aa, Homo sapiens
GFSAKEAQDTSDGIIQK

SEQ ID NO: 443 - protein, 12 aa, Homo sapiens
VTAMDVVYALKR

SEQ ID NO: 444 - protein, 14 aa, Homo sapiens
SYPARVPPPPPIAR

SEQ ID NO: 445 - protein, 16 aa, Homo sapiens
SYELPDGQVITIGNER

SEQ ID NO: 446 - protein, 15 aa, Homo sapiens
SVYYNEATGGKYVPR

SEQ ID NO: 447 - protein, 16 aa, Homo sapiens
SVPRGEAAGAVQELAR

SEQ ID NO: 448 - protein, 13 aa, Homo sapiens
SLTTIPELKDHLR

SEQ ID NO: 449 - protein, 18 aa, Homo sapiens
SLTPAVPVESKPDKPSGK

SEQ ID NO: 450 - protein, 9 aa, Homo sapiens
SLQSVAEER

SEQ ID NO: 451 - protein, 14 aa, Homo sapiens
SLPGEQEQEVAGSK

SEQ ID NO: 452 - protein, 11 aa, Homo sapiens
SKLNYKPPPQK

SEQ ID NO: 453 - protein, 14 aa, Homo sapiens
SISSQLGPIHPPPR

SEQ ID NO: 454 - protein, 16 aa, Homo sapiens
SHHAASTTTAPTPAAR

SEQ ID NO: 455 - protein, 11 aa, Homo sapiens
SGPPVSELITK

SEQ ID NO: 456 - protein, 12 aa, Homo sapiens
SFQTSPSTESLK

SEQ ID NO: 457 - protein, 13 aa, Homo sapiens
SFPTQDHLPATPR

SEQ ID NO: 458 - protein, 14 aa, Homo sapiens
SFGGDAQADEGQAR

SEQ ID NO: 459 - protein, 9 aa, Homo sapiens
SFFTPGKPK

SEQ ID NO: 460 - protein, 19 aa, Homo sapiens
SAVGTLPATSPQSTSVQAK

SEQ ID NO: 461 - protein, 12 aa, Homo sapiens
SAPGGGSKVPQK

SEQ ID NO: 462 - protein, 13 aa, Homo sapiens
SAPATGGVKKPHR

SEQ ID NO: 463 - protein, 19 aa, Homo sapiens
NSPSTTPPTVTTNMPVTNR

SEQ ID NO: 464 - protein, 18 aa, Homo sapiens
MWISKQEYDESGPSIVHR

SEQ ID NO: 465 - protein, 18 aa, Homo sapiens
MVSPFHSPPSTPSSPGVR

SEQ ID NO: 466 - protein, 12 aa, Homo sapiens
MIASDSHRPEVK

SEQ ID NO: 467 - protein, 19 aa, Homo sapiens
MAPVPLDDSNRPASLTKDR

SEQ ID NO: 468 - protein, 15 aa, Homo sapiens
MVLLESEQFLTELTR

SEQ ID NO: 469 - protein, 13 aa, Homo sapiens
LVQSPNSYFMDVK

SEQ ID NO: 470 - protein, 16 aa, Homo sapiens
LQSAHPGEHLAQGASR

SEQ ID NO: 471 - protein, 13 aa, Homo sapiens
KASGPPVSELITK

SEQ ID NO: 472 - protein, 11 aa, Homo sapiens
IWHHTFYNELR

SEQ ID NO: 473 - protein, 20 aa, Homo sapiens
GVTQFGNKYIQQTKPLTLER

SEQ ID NO: 474 - protein, 18 aa, Homo sapiens
GVPSDSVEAAKNASNTEK

SEQ ID NO: 475 - protein, 18 aa, Homo sapiens
GVPLDATEDSKKNEPIFK

SEQ ID NO: 476 - protein, 13 aa, Homo sapiens
GVAATPGKAEATR

SEQ ID NO: 477 - protein, 12 aa, Homo sapiens
GSSPLLDIVGGR

SEQ ID NO: 478 - protein, 15 aa, Homo sapiens
GSETPQLFTVLPEKR

SEQ ID NO: 479 - protein, 11 aa, Homo sapiens
GLPEEQPQTTK

SEQ ID NO: 480 - protein, 11 aa, Homo sapiens
GLLPTPDEFPR

SEQ ID NO: 481 - protein, 17 aa, Homo sapiens
GLGVARPHYGSVLDNER

SEQ ID NO: 482 - protein, 12 aa, Homo sapiens
GISAGAVQTAGK

SEQ ID NO: 483 - protein, 17 aa, Homo sapiens
GINYQPPTVVPGGDLAK

SEQ ID NO: 484 - protein, 15 aa, Homo sapiens
GGGPGQVVDDGLEHR

SEQ ID NO: 485 - protein, 17 aa, Homo sapiens
GFFWTQGSPKPGTASPK

SEQ ID NO: 486 - protein, 10 aa, Homo sapiens
FVSEAELDER

SEQ ID NO: 487 - protein, 7 aa, Homo sapiens
FFSALEK

SEQ ID NO: 488 - protein, 18 aa, Homo sapiens
AYEPQGGSGYDYSYAGGR

SEQ ID NO: 489 - protein, 17 aa, Homo sapiens
AVPKEDIYSGGGGGGSR

SEQ ID NO: 490 - protein, 20 aa, Homo sapiens
AVPIAQKSEPHSLSSEALMR

SEQ ID NO: 491 - protein, 11 aa, Homo sapiens
AVFPSIVGRPR

SEQ ID NO: 492 - protein, 11 aa, Homo sapiens
ATVTPSPVKGK

SEQ ID NO: 493 - protein, 15 aa, Homo sapiens
ASSASSFLDSDELER

SEQ ID NO: 494 - protein, 15 aa, Homo sapiens
AMEELDGDDVRVSSR

SEQ ID NO: 495 - protein, 11 aa, Homo sapiens
ALYVACQGQPK

SEQ ID NO: 496 - protein, 12 aa, Homo sapiens
ALPSHLGLHPER

SEQ ID NO: 497 - protein, 15 aa, Homo sapiens
ALLNLPGTQTSGEAK

SEQ ID NO: 498 - protein, 15 aa, Homo sapiens
ALIGDDVGLTSYKHR

SEQ ID NO: 499 - protein, 14 aa, Homo sapiens
AITGASLADIMAKR

SEQ ID NO: 500 - protein, 12 aa, Homo sapiens
AAGYDVEKNNSR

SEQ ID NO: 501 - protein, 13 aa, Homo sapiens
FVVPVASPSGDAR

SEQ ID NO: 502 - protein, 9 aa, Homo sapiens
AAAGAPLPR

SEQ ID NO: 503 - protein, 17 aa, Homo sapiens
GLGPQGFPELKNDTFLR

SEQ ID NO: 504 - protein, 15 aa, Homo sapiens
AAPEEESAYVAGEKR

SEQ ID NO: 505 - protein, 9 aa, Homo sapiens
SLPEAGPGR

SEQ ID NO: 506 - protein, 12 aa, Homo sapiens
GLSPLSSPSDTK

SEQ ID NO: 507 - protein, 12 aa, Homo sapiens
AAGVTDGNEVAK

SEQ ID NO: 508 - protein, 10 aa, Homo sapiens
VFIGINDLEK

SEQ ID NO: 509 - protein, 13 aa, Homo sapiens
AITGASLADIMAK

SEQ ID NO: 510 - protein, 12 aa, Homo sapiens
GIVPDIAVGTKR

SEQ ID NO: 511 - protein, 15 aa, Homo sapiens
AASQLNVDASGNLAK

SEQ ID NO: 512 - protein, 8 aa, Homo sapiens
MLDDIVSR

SEQ ID NO: 513 - protein, 11 aa, Homo sapiens
GFDVASVQQQR

SEQ ID NO: 514 - protein, 17 aa, Homo sapiens
AAPELPVPTGGPAVGAR

SEQ ID NO: 515 - protein, 12 aa, Homo sapiens
SVVSFDKVKEPR

SEQ ID NO: 516 - protein, 18 aa, Homo sapiens
GLAVTPTPVPVVGSQMTR

SEQ ID NO: 517 - protein, 12 aa, Homo sapiens
ALAEGPGAEGPR

SEQ ID NO: 518 - protein, 17 aa, Homo sapiens
GQSDENKDDYTIPDEYR

SEQ ID NO: 519 - protein, 16 aa, Homo sapiens
GFAEAIHSPQVAGVPR

SEQ ID NO: 520 - protein, 11 aa, Homo sapiens
AVPSPPPASPR

SEQ ID NO: 521 - protein, 16 aa, Homo sapiens
GISSSNEGVEEPSKKR

SEQ ID NO: 522 - protein, 9 aa, Homo sapiens
GIGTVPVGR

SEQ ID NO: 523 - protein, 12 aa, Homo sapiens
ALPGDNVGFNVK

SEQ ID NO: 524 - protein, 15 aa, Homo sapiens
GLVETPTGYIESLPR

SEQ ID NO: 525 - protein, 20 aa, Homo sapiens
SLLEPRDPVASSLSPYFGTK

SEQ ID NO: 526 - protein, 13 aa, Homo sapiens
SISESVPVGPKVR

SEQ ID NO: 527 - protein, 11 aa, Homo sapiens
SISESVPVGPK

SEQ ID NO: 528 - protein, 11 aa, Homo sapiens
GVPSDSVEAAK

SEQ ID NO: 529 - protein, 7 aa, Homo sapiens
AINTEFK

SEQ ID NO: 530 - protein, 14 aa, Homo sapiens
SLADAINTEFKNTR

SEQ ID NO: 531 - protein, 12 aa, Homo sapiens
ASGPPVSELITK

SEQ ID NO: 532 - protein, 11 aa, Homo sapiens
ALAAAGYDVEK

SEQ ID NO: 533 - protein, 15 aa, Homo sapiens
ALAAAGYDVEKNNSR

SEQ ID NO: 534 - protein, 20 aa, Homo sapiens
GVTHTVPIYEGYALPHAILR

SEQ ID NO: 535 - protein, 17 aa, Homo sapiens
GMGQKDSYVGDEAQSKR

SEQ ID NO: 536 - protein, 9 aa, Homo sapiens
GFAGDDAPR

SEQ ID NO: 537 - protein, 10 aa, Homo sapiens
LLTEAPLNPK

SEQ ID NO: 538 - protein, 10 aa, Homo sapiens
AGFAGDDAPR

SEQ ID NO: 539 - protein, 18 aa, Homo sapiens
VAPEEHPVLLTEAPLNPK

SEQ ID NO: 540 - protein, 19 aa, Homo sapiens
ALDFEQEMATAASSSSLEK

SEQ ID NO: 541 - protein, 10 aa, Homo sapiens
GASQFQEVIR

SEQ ID NO: 542 - protein, 10 aa, Homo sapiens
ALGSPEMDVR

SEQ ID NO: 543 - protein, 12 aa, Homo sapiens
GMTELEPSKFSK

SEQ ID NO: 544 - protein, 9 aa, Homo sapiens
GLPTGAEGR

SEQ ID NO: 545 - protein, 9 aa, Homo sapiens
GFDQNVNVK

SEQ ID NO: 546 - protein, 10 aa, Homo sapiens
YIPAENSPTR

SEQ ID NO: 547 - protein, 10 aa, Homo sapiens
SISESAFSAR

SEQ ID NO: 548 - protein, 12 aa, Homo sapiens
AAVQAAILSGDK

SEQ ID NO: 549 - protein, 14 aa, Homo sapiens
ALLQTDQSLSEKEK

SEQ ID NO: 550 - protein, 15 aa, Homo sapiens
ALAAGGYDVEKNNSR

SEQ ID NO: 551 - protein, 13 aa, Homo sapiens
GSSPLLDIVGGRK

SEQ ID NO: 552 - protein, 7 aa, Homo sapiens
AVPIAQK

SEQ ID NO: 553 - protein, 13 aa, Homo sapiens
SIFQHIQSAQSQR

SEQ ID NO: 554 - protein, 14 aa, Homo sapiens
SLRPDPNFDALISK

SEQ ID NO: 555 - protein, 12 aa, Homo sapiens
EHGLAPAPTTIR

SEQ ID NO: 556 - protein, 14 aa, Homo sapiens
WTYHYSEKPMNWQR

SEQ ID NO: 557 - protein, 10 aa, Homo sapiens
GLLLLGSGSR

SEQ ID NO: 558 - protein, 14 aa, Homo sapiens
SVPAAEPEYPKGIR

SEQ ID NO: 559 - protein, 11 aa, Homo sapiens
SVPAAEPEYPK

SEQ ID NO: 560 - protein, 16 aa, Homo sapiens
GLGLSYLSSHIANVER

SEQ ID NO: 561 - protein, 10 aa, Homo sapiens
AINTEFKNTR

SEQ ID NO: 562 - protein, 11 aa, Homo sapiens
ALKGTNESLER

SEQ ID NO: 563 - protein, 23 aa, Homo sapiens
AEIVGGHEAQPHSRPYMASLQMR

SEQ ID NO: 564 - protein, 16 aa, Homo sapiens
SMPPAQQQITSGQMHR

SEQ ID NO: 565 - protein, 13 aa, Homo sapiens
MVMEKPSPLLVGR

SEQ ID NO: 566 - protein, 9 aa, Homo sapiens
MMLDDIVSR

SEQ ID NO: 567 - protein, 10 aa, Homo sapiens
MKETIMNQEK

SEQ ID NO: 568 - protein, 12 aa, Homo sapiens
MVMAEGTAVLRR

SEQ ID NO: 569 - protein, 11 aa, Homo sapiens
AMLDQLMGTSR

SEQ ID NO: 570 - protein, 10 aa, Homo sapiens
MVNFTVDQIR

SEQ ID NO: 571 - protein, 17 aa, Homo sapiens
MGLLSQGSPLSWEETKR

SEQ ID NO: 572 - protein, 14 aa, Homo sapiens
MGVQVETISPGDGR

SEQ ID NO: 573 - protein, 28 aa, Homo sapiens
AYFEKVGDTSLDPNDFDFTVTGRGSPSR

SEQ ID NO: 574 - protein, 23 aa, Homo sapiens
VFDNGSIYNPEVLDITEETLHSR

SEQ ID NO: 575 - protein, 21 aa, Homo sapiens
SIGASPNPFSVHTATAVPSGK

SEQ ID NO: 576 - protein, 33 aa, Homo sapiens
KVDEGAGDSAAVASGGAQTLALAGSPAPSGHPK

SEQ ID NO: 577 - protein, 22 aa, Homo sapiens
GSDASQLLHQAEVAQQEFLEVK

SEQ ID NO: 578 - protein, 23 aa, Homo sapiens
AVTPGPQPTLEQLEEGGPRPLER

SEQ ID NO: 579 - protein, 29 aa, Homo sapiens
AVSGQLPDPTTNPSAGKDGPSLLVVEQVR

SEQ ID NO: 580 - protein, 23 aa, Homo sapiens
ALVEFESNPEETREPGSPPSVQR

SEQ ID NO: 581 - protein, 21 aa, Homo sapiens
GVPVPGSPFPLEAVAPTKPSK

SEQ ID NO: 582 - protein, 23 aa, Homo sapiens
GQHPAQEEVPESPQTSGPEAENR

SEQ ID NO: 583 - protein, 23 aa, Homo sapiens
SAHPEEGDLDLASESTAHAQSSK

SEQ ID NO: 584 - protein, 28 aa, Homo sapiens
GVPSDSVEAAKNASNTEKLTDQVMQNPR

SEQ ID NO: 585 - protein, 23 aa, Homo sapiens
AAPAPAPPPEPERPKEVEFDASK

SEQ ID NO: 586 - protein, 21 aa, Homo sapiens
ATVGGPAPTPLLPPSATASVK

SEQ ID NO: 587 - protein, 26 aa, Homo sapiens
GVQLPPGDYSTTPGGTLFSTTPGGTR

SEQ ID NO: 588 - protein, 24 aa, Homo sapiens
SLAGSSGPGASSGTSGDHGELVVR

SEQ ID NO: 589 - protein, 17 aa, Homo sapiens
SFSDADLADGVSGGEGK

SEQ ID NO: 590 - protein, 11 aa, Homo sapiens
SSASSGPQILK

SEQ ID NO: 591 - protein, 10 aa, Homo sapiens
LYQTIEENIK